(19) United States
(12) Patent Application Publication
(10) Pub. No.: US 2025/0146020 A1
(43) Pub. Date: May 8, 2025

(54) Stable Cell Lines for Inducible Production of rAAV Virions

(71) Applicant: Shape Therapeutics Inc., Seattle, WA (US)

(72) Inventors: Kenneth Prentice, Seattle, WA (US); Sandhya Pande, Kent, WA (US)

(21) Appl. No.: 18/750,819

(22) Filed: Jun. 21, 2024

Related U.S. Application Data
(63) Continuation of application No. 17/390,777, filed on Jul. 30, 2021, now Pat. No. 12,054,738.
(60) Provisional application No. 63/216,615, filed on Jun. 30, 2021.
(60) Provisional application No. 63/156,239, filed on Mar. 3, 2021.
(60) Provisional application No. 63/156,230, filed on Mar. 3, 2021.
(60) Provisional application No. 63/156,207, filed on Mar. 3, 2021.
(60) Provisional application No. 63/058,900, filed on Jul. 30, 2020.
(60) Provisional application No. 63/058,894, filed on Jul. 30, 2020.
(60) Provisional application No. 63/058,887, filed on Jul. 30, 2020.

(51) Int. Cl. C12N 15/864; C07K 14/005

(52) U.S. Cl. CPC C12N 15/8645; C07K 14/005; C12N 2750/14143; C12N 2750/14152

(57) ABSTRACT

Described herein are polynucleotide constructs and stable cell lines for inducible production of rAAV virions within which are packaged a payload polynucleotide.

### 1. CROSS-REFERENCE TO RELATED APPLICATIONS

This application is a continuation of U.S. patent application Ser. No. 17/390,777, filed Jul. 30, 2021, which claims the benefit of and priority to U.S. Provisional Application Nos. 63/058,887, filed Jul. 30, 2020; 63/058,894, filed Jul. 30, 2020; 63/058,900, filed Jul. 30, 2020; 63/156,230, filed Mar. 3, 2021; 63/156,207, filed Mar. 3, 2021, 63/156,239; filed Mar. 3, 2021; and 63/216,615, filed Jun. 30, 2021. The content of each of the above-referenced applications is incorporated by reference in its entirety.

### 2. INCORPORATION BY REFERENCE OF SEQUENCE LISTING

The present application is being filed along with a Sequence Listing in electronic format. The Sequence Listing is provided as a file entitled 324632000101SeqList.xml, created Jan. 15, 2025, which is 279,621 bytes in size. The information in the electronic format of the Sequence Listing is herein incorporated by reference in its entirety.

### 3. BACKGROUND

Recombinant adeno-associated virus (rAAV) is the preferred vehicle for in vivo gene delivery. AAV has no known disease associations, infects dividing and non-dividing cells, rarely if ever integrates into the mammalian cell genome, and can persist essentially for the lifetime of infected cells as a transcriptionally active nuclear episome. The FDA has recently approved several rAAV gene therapy products and many other rAAV-based gene therapy and gene editing products are in development.

The most widely used method for producing rAAV virions is based on the helper-virus-free transient transfection of multiple plasmids, typically a triple transfection, into adherent cell lines. Although there is ongoing investment to increase production capacity, current AAV manufacturing processes are inefficient and expensive. In addition, they result in variable product quality, with low levels of encapsidation of a payload, such as a therapeutic payload.

There is, therefore, a need for improved methods for producing rAAV products. Any such solution must address the toxicity to the host production cell due to constitutive expression of AAV Rep protein and the toxicity to the host production cell due to constitutive expression of adenoviral helper protein.

### 4. SUMMARY

Disclosed herein are stable mammalian cell lines, wherein the cells are capable of conditionally producing recombinant AAV (rAAV) virions within which are packaged an expressible payload.

Further provided herein is a stable mammalian cell line, wherein the cells are capable of conditionally producing recombinant AAV (rAAV) virions within which are packaged an expressible payload; and wherein a population of virions produced by the stable cell are more homogenous than a population of virions produced by an otherwise comparable cell producing rAAV virions upon transient transfection.

Further provided herein is a stable mammalian cell line, wherein the cells are capable of conditionally producing recombinant AAV (rAAV) virions within which are packaged an expressible payload; and production of virions is inducible upon addition of a triggering agent.

Further provided herein is a stable mammalian cell line, wherein the cells are capable of conditionally producing recombinant AAV (rAAV) virions within which are packaged an expressible payload; and production of virions is not conditioned on the presence of a plasmid within the cell.

In some aspects, a composition comprising one or more nucleic acids which together comprises: (i) a first recombinant nucleic acid sequence encoding an AAV Rep protein and an AAV Cap protein; and (ii) a second recombinant nucleic acid sequence encoding one or more adenoviral helper proteins, wherein when the one or more nucleic acids are integrated into the nuclear genome of a mammalian cell the AAV Rep protein, the AAV Cap protein, and/or the one or more adenoviral helper proteins are conditionally expressible and thereby conditionally produce recombinant AAV (rAAV) virions. In some embodiments, the conditional expression of the AAV Rep protein, the AAV Cap protein, and/or the one or more adenoviral helper proteins is controlled by one or more excisable elements present in the one or more nucleic acids. In some embodiments, the one or more excisable elements comprise one or more introns and/or one or more exons. In some embodiments, the first recombinant nucleic acid sequence encodes: a) a first part of the AAV Rep protein coding sequence; b) the second part of the AAV Rep protein coding sequence; c) an excisable element between the first part of the AAV Rep protein coding sequence and the second part of the AAV Rep protein coding sequence; and d) the AAV Cap protein coding sequence. In some embodiments, the excisable element comprises: a) a first spacer segment comprising a first intron, b) a second spacer segment comprising a coding sequence of a detectable marker; and c) a third spacer segment comprising a second intron, and wherein the first spacer segment and the third spacer segment are capable of being excised by endogenous cellular machinery of a mammalian cell. In some embodiments, the excisable element comprises from 5′ to 3′: a) a 5′ splice site; b) a first spacer segment comprising a first intron; c) a second spacer segment comprising: i) a first lox sequence; ii) a 3′ splice site; iii) an exon; iv) a stop signaling sequence; and v) a second lox sequence; and d) a third spacer segment comprising a second intron. In some embodiments, the detectable marker is a luminescent marker, a radiolabel or a fluorescent marker, optionally a fluorescent marker which is GFP, EGFP, RFP, CFP, BFP, YFP, or mCherry. In some embodiments, a) the first spacer segment comprises a nucleic acid sequence having at least 80% identity to SEQ ID NO: 1; and/or b) the second spacer segment comprises a nucleic acid sequence having at least 80% identity to SEQ ID NO: 2; and/or c) the third spacer segment comprises a nucleic acid sequence having at least 80% identity to SEQ ID NO: 3. In some embodiments, the second spacer segment is capable of being excised by a Cre polypeptide. In some embodiments, the expression of the AAV Rep protein and/or the AAV Cap protein is driven by native promoters. In some embodiments, wherein: a) the native promoters P5 and/or P19 drive the expression of the AAV Rep protein; and/or b) the native promoter P40 drives the expression of the AAV Cap protein. In some embodiments, the second recombinant nucleic acid sequence encodes: a) one or more adenoviral helper proteins; b) a conditionally self-excising element; and c) an inducible promoter; wherein, once integrated into the nuclear genome of a mammalian cell, the expression of the one or more adenoviral helper protein coding sequences is under the control of the conditionally self-excising element and the inducible promoter. In some embodiments, the one or more adenoviral helper proteins comprise E2A and E4. In some embodiments, the self-excising element comprises a sequence which encodes a polypeptide, preferably a recombinase polypeptide, more preferably a Cre polypeptide. In some embodiments, the polypeptide encoded by the self-excising element is conditionally expressible and is expressed only in the presence of a triggering agent. In some embodiments, the triggering agent is a hormone, preferably tamoxifen. In some embodiments, the inducible promoter is a Tet inducible promoter. In some embodiments, the second recombinant nucleic acid sequence further comprises a sequence that encodes a Tet responsive activator protein, preferably Tet-on-3G. In some embodiments, the expression of Tet-On 3G activator protein is driven by an Elalpha promoter. In some embodiments, the second recombinant nucleic acid sequence comprises a sequence with at least 80% homology, at least 90% homology, at least 95% homology, at least 99% homology, or a sequence identical to SEQ ID NO: 11 or SEQ ID NO: 12. In some embodiments, the one or more nucleic acids further comprises a nucleic acid sequence encoding a VA RNA sequence. In some embodiments, the expression of VA RNA is constitutive. In some embodiments, the expression of VA RNA is inducible. In some embodiments, the VA RNA sequence comprises one or more mutations in the VA RNA internal promoter, preferably G16A and G60A. In some embodiments, the expression of VA RNA is driven by a Elalpha promoter or a U6 promoter. In some embodiments, the expression of VA RNA is driven by a U6 promoter, and wherein the U6 promoter comprises: a) a first part of a U6 promoter sequence, b) a stuffer sequence, and c) a second part of a U6 promoter sequence, and wherein the stuffer sequence is capable of being excised by a Cre polypeptide. In some embodiments, a serotype of the AAV Cap protein is selected from the group consisting of AAV1, AAV2, AAV3, AAV4, AAV5, AAV6, AAV7, AAV8, AAV9, AAV 10, AAV11, AAV 12, AAV13, AAV 14, AAV 15 and AAV 16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16 and AAVhu68. In some embodiments, the serotype is an AAV5 and the Cap protein that comprises one or more mutations or insertions. In some embodiments, the one or more recombinant nucleic acids further encode a third recombinant nucleic acid sequence encoding a payload, optionally wherein the payload is: (a) a polynucleotide payload, such as a guide RNA for RNA editing, a guide RNA for Cas protein-directed DNA editing, a tRNA suppressor, or a gene for replacement gene therapy; or (b) a protein such as a therapeutic antibody or a vaccine immunogen. In some embodiments, the one or more recombinant nucleic acids comprise one or more mammalian cell selection elements. In some embodiments, one or more of the mammalian cell selection elements encodes an antibiotic resistance gene, optionally a blasticidin resistance gene. In some embodiments, one or more of the mammalian cell selection elements is an auxotrophic selection element which encodes an active protein, preferably wherein the protein is DHFR. In some embodiments, one or more of the mammalian cell selection elements is a first auxotrophic selection element which encodes an inactive protein that requires expression of a second inactive protein from a second auxotrophic selection coding sequence for activity In some embodiments, the first auxotrophic selection coding sequence encodes for DHFR Z-Cter (SEQ ID NO: 5) activity, and/or wherein the second auxotrophic selection coding sequence encodes for DHFR Z-Nter (SEQ ID NO: 4). In some embodiments, a) the first recombinant nucleic acid comprises a mammalian cell selection element which encodes an antibiotic resistance gene, preferably a blasticidin resistance gene; and b) i. the second recombinant nucleic acid comprises a first auxotrophic selection element which encodes an inactive protein that requires expression of a second inactive protein from a second auxotrophic selection coding sequence for activity; and ii. the third recombinant nucleic acid comprises the second auxotrophic selection element which encodes the inactive protein that requires expression of the first inactive protein from the first auxotrophic selection coding sequence for activity; and wherein in (i) or (ii) the first auxotrophic selection coding sequence encodes for DHFR Z-Cter (SEQ ID NO: 5), and the second auxotrophic selection coding sequence encodes for DHFR Z-Nter (SEQ ID NO: 4) or wherein the first auxotrophic selection coding sequence encodes for DHFR Z-Nter (SEQ ID NO: 4), and the second auxotrophic selection coding sequence encodes for DHFR Z-Cter (SEQ ID NO: 5).

In some aspects, disclosed herein is a mammalian cell wherein the nuclear genome of the cell comprises a plurality of integrated recombinant nucleic acid constructs which together encode for a recombinant adeno-associated virus (rAAV) virions, wherein the rAAV virions can be conditionally expressed from the cell. In some embodiments, the plurality of integrated recombinant nucleic acid constructs comprise the one or more recombinant nucleic acids of any one of previous embodiment, wherein the AAV Rep protein, the AAV Cap protein and/or the adenoviral helper proteins can be conditionally expressed from the cell. In some embodiments, the cell line expresses adenoviral helper proteins E1A and E1B. In some embodiments, the plurality of integrated recombinant nucleic acid constructs comprise: (i) a first integrated polynucleotide construct comprising: a) a first part of an AAV Rep protein coding sequence; b) a second part of an AAV Rep protein coding sequence; c) an excisable element between the first part of the AAV Rep protein coding sequence and the second part of the AAV Rep protein coding sequence, wherein the excisable element comprises: i) a first spacer segment comprising a first intron; ii) a second spacer segment comprising a coding sequence of a detectable marker, wherein the second spacer segment is capable of being excised by a Cre polypeptide; and iii) a third spacer segment comprising a second intron; and d) an AAV Cap protein coding sequence; wherein the AAV Rep protein and the AAV Cap protein is driven by the native promoters P5, P19, and P40; (ii) a second integrated polynucleotide construct comprising a) a conditionally expressible VA RNA coding sequence which comprises a mutation in the VA RNA internal promoter, wherein the expression of VA RNA is driven by a U6 promoter, optionally wherein the VA RNA sequence comprises G16A and G60A mutations; b) one or more adenoviral helper protein coding sequences, wherein the adenoviral helper proteins are E2A and E4; c) a conditionally self-excising element which encodes a Cre polypeptide which translocates to the nucleus and self-excises only in the presence of a triggering agent which is tamoxifen, and d) an inducible promoter which is a Tet inducible promoter, and wherein the expression of the one or more adenoviral helper protein coding sequences is under the control of the conditionally self-excising element and the inducible promoter; and (iii) a third integrated polynucleotide construct comprising encodes for the payload, wherein the payload is a polynucleotide payload.

In some aspects, a method of producing a population of rAAV virions comprises: (a) culturing the cell of any one of aspects 36-39 in conditions which allow for the expression of the rAAV virions; and (b) isolating the rAAV virions from the cell culture.

In some embodiments, the prepurification rAAV viral genome (VG) to viral particle (VG:VP) ratio of greater than 0.5. In some embodiments, the population of rAAV virions produced by the cell has: (a) a ratio of viral genomes to transduction units of about 500 to 1 to 1 to 1; and/or (b) a ratio of vector genomes to infectious unit of 100:1.

In some aspects, a method of preparing the cell of any one of the previous embodiments comprises: i) providing a mammalian cell and the one or more nucleic acids of any one of the previous embodiments; and ii) integrating the one or more nucleic acids of any one of the previous embodiments into the nuclear genome of the mammalian cell.

In some aspects, a population of rAAV virions produced by the method of any one of the previous embodiments. In some embodiments, the infectivity of the virions is at least 50% at an MOI of 10000.

In some aspects, a pharmaceutical composition comprising a population of rAAV virions according to any one of the previous embodiments, for use as a medicament, optionally for use in treating a monogenic disorder. In some embodiments, the population of rAAV virions according to any one of the previous embodiments or the pharmaceutical composition according to any one of the previous embodiments, for use as a medicament, optionally for use in treating a monogenic disorder. In some embodiments, the population of rAAV virions or the pharmaceutical composition for use according to any one of the previous embodiments, wherein the rAAV virions are administered at a dosage of 4×1014 or lower.

Also provided herein are cells comprising: a) a first polynucleotide construct coding for an AAV Rep protein and an AAV Cap protein; b) a second polynucleotide construct coding for one or more adenoviral helper proteins; wherein when the one or more nucleic acids are integrated into the nuclear genome of a mammalian cell the AAV Rep protein, the AAV Cap protein, and/or the one or more adenoviral helper proteins are conditionally expressible and thereby conditionally produce recombinant AAV (rAAV) virions.

In some embodiments, the second polynucleotide construct comprises a sequence coding for: a) one or more helper proteins; b) a self-excising element upstream of the one or more helper proteins; and c) an inducible promoter upstream of the self-excising element. In some embodiments, the self-excising element is operably linked to the inducible promoter. In some embodiments, expression of the self-excising element is driven by the inducible promoter.

In some embodiments, the inducible promoter is a tetracycline-responsive promoter element (TRE). In some embodiments, the TRE comprises Tet operator (tetO) sequence concatemers fused to a minimal promoter. In some embodiments, the minimal promoter is a human cytomegalovirus promoter. In some embodiments, the inducible promoter comprises a sequence having at least 70%, 80%, 90%, 95%, 99%, or 100% sequence identity to SEQ ID NO: 22. In some embodiments, transcription is activated from the inducible promoter upon binding of an activator. In some embodiments, the activator binds to the inducible promoter in the presence of a first triggering agent. In some embodiments, the second polynucleotide construct further comprises a sequence coding for an activator. In some embodiments, the activator is operably linked to a constitutive promoter. In some embodiments, the constitutive promoter is Elalpha promoter or human cytomegalovirus promoter. In some embodiments, the E1 alpha promoter comprises at least one mutation. In some embodiments, the constitutive promoter comprises a sequence having at least 70%, 80%, 90%, 95%, 99%, or 100% sequence identity with SEQ ID NO: 20. In some embodiments, the activator is reverse tetracycline-controlled transactivator (rTA) comprising a Tet Repressor binding protein (TetR) fused to a VP16 transactivation domain. In some embodiments, the rTA comprises four mutations in the tetR DNA binding moiety. In some embodiments, the rTA comprises a sequence having at least 70%, 80%, 90%, 95%, 99%, or 100% sequence identity to SEQ ID NO: 21.

In some embodiments, the inducible promoter is a cumate operator sequence. In some embodiments, the cumate operator sequence is downstream of a constitutive promoter. In some embodiments, the constitutive promoter is a human cytomegalovirus promoter. In some embodiments, the inducible promoter is bound by a cymR repressor in the absence of a first triggering agent. In some embodiments, the inducible promoter is activated in the presence of a first triggering agent. In some embodiments, the first triggering agent binds to the cymR repressor. In some embodiments, the second polynucleotide construct further comprises a cymR repressor. In some embodiments, the cymR repressor is operably linked to a constitutive promoter. In some embodiments, the constitutive promoter is Elalpha promoter. In some embodiments, the E1 alpha promoter comprises at least one mutation. In some embodiments, the constitutive promoter comprises a sequence having at least 70%, 80%, 90%, 95%, 99%, or 100% sequence identity with SEQ ID NO: 20. In some embodiments, the first triggering agent is a cumate.

In some embodiments, the sequence coding for the self-excising element comprises a poly A sequence. In some embodiments, the self-excising element is a recombinase. In some embodiments, the recombinase is fused to a ligand binding domain. In some embodiments, the recombinase is Cre polypeptide or flippase polypeptide. In some embodiments, the Cre polypeptide is fused to a ligand binding domain. In some embodiments, the ligand binding domain is a hormone receptor. In some embodiments, the recombinase is a Cre-ERT2 polypeptide. In some embodiments, the self-excising element translocates to the nucleus in the presence of a second triggering agent. In some embodiments, the second triggering agent is an estrogen receptor ligand. In some embodiments, the second triggering agent is a selective estrogen receptor modulator (SERM). In some embodiments, the second triggering agent is tamoxifen. In some embodiments, the recombinase is flanked by recombination sites. In some embodiments, the recombination sites are lox sites or flippase recognition target (FRT) sites. In some embodiments, the lox sites are loxP sites.

In some embodiments, the one or more adenoviral helper proteins comprise E2A and E4. In some embodiments, the E2A is FLAG-tagged E2A. In some embodiments, the sequence coding for E2A and the sequence coding for E4 are separated by an internal ribosome entry site (IRES) or by P2A.

In some embodiments, the second polynucleotide construct further comprises a sequence coding for a selectable marker. In some embodiments, the selectable marker is an antibiotic resistance protein. In some embodiments, the selectable marker is a split intein linked to an N-terminus of the antibiotic resistance protein or split intein linked to a C-terminus of the antibiotic resistance protein. In some embodiments, the antibiotic resistance protein is for puromycin resistance or blasticidin resistance.

In some embodiments, the second polynucleotide construct further comprises a sequence coding for VA RNA. In some embodiments, the sequence coding for VA RNA is a transcriptionally dead sequence. In some embodiments, the sequence coding for VA RNA comprises at least two mutations in the internal promoter. In some embodiments, expression of VA RNA is driven by a U6 promoter. In some embodiments, the second polynucleotide construct further comprises upstream of the sequence coding for VA RNA gene sequence, from 5′ to 3′: a) a first part of a U6 promoter sequence; b) a first recombination site; c) a stuffer sequence; d) a second recombination site; and e) a second part of a U6 promoter sequence. In some embodiments, the stuffer sequence is excisable by the recombinase. In some embodiments, the stuffer sequence comprises a sequence encoding a gene. In some embodiments, the stuffer sequence comprises a promoter. In some embodiments, the promoter is a constitutive promoter. In some embodiments, the promoter is a CMV promoter.

In some embodiments, the first polynucleotide construct comprises: a) a sequence of a first part of a Rep gene; b) a sequence of a second part of the Rep gene; c) a sequence of a Cap gene; and d) an excisable element positioned between the first part of the sequence of Rep gene and the second part of the sequence of the Rep gene.

In some embodiments, the excisable element comprises a stop signaling sequence. In some embodiments, the excisable element comprises a rabbit beta globin intron. In some embodiments, the excisable element comprises an exon. In some embodiments, the excisable element comprises an intron and an exon. In some embodiments, the excisable element comprises an intron.

In some embodiments, two splice sites are positioned between the sequence of the first part of the Rep gene and the sequence of the second part of the Rep gene. In some embodiments, the two splice sites are a 5′ splice site and a 3′ splice site. In some embodiments, the 5′ splice site is a rabbit beta globin 5′ splice site. In some embodiments, the 3′ splice site is a rabbit beta globin 3′ splice site. In some embodiments, three splice sites are positioned between the sequence of the first part of the Rep gene and the sequence of the second part of the Rep gene. In some embodiments, the three splice sites are a 5′ splice site, a first 3′ splice site, and a second 3′ splice site. In some embodiments, a first 3′ splice site is a duplicate of the second 3′ splice site. In some embodiments, the first 3′ splice site is a rabbit beta globin 3′ splice site. In some embodiments, the second 3′ splice site is a rabbit beta globin 3′ splice site.

In some embodiments, the excisable element comprises a recombination site. In some embodiments, the recombination site is a lox site or FRT site. In some embodiments, the lox site is a loxP site.

In some embodiments, the excisable element comprises from 5′ to 3′: a) the 5′ splice site; b) a first recombination site; c) the first 3′ splice site; d) a stop signaling sequence; e) a second recombination site; and f) the second 3′ splice site.

In some embodiments, the excisable element comprises from 5′ to 3′: a) the 5′ splice site; b) a first spacer segment; c) a second spacer segment comprising: i) a first recombination site; ii) the first 3′ splice site; iv) a stop signaling sequence; and v) a second recombination site; and d) a third spacer segment comprising the second 3′ splice site. In some embodiments, the first spacer sequence comprises an intron. In some embodiments, the first spacer segment comprises a sequence having at least 70%, 80%, 90%, 95%, 99%, or 100% sequence identity to SEQ ID NO: 1. In some embodiments, the second spacer segment comprises a sequence having at least 70%, 80%, 90%, 95%, 99%, or 100% sequence identity to SEQ ID NO: 2. In some embodiments, the third spacer segment comprises a sequence having at least 70%, 80%, 90%, 95%, 99%, or 100% sequence identity to SEQ ID NO: 3. In some embodiments, the third spacer segment comprises an intron. In some embodiments, the first spacer segment and the third spacer segment are capable of being excised by endogenous cellular machinery. In some embodiments, the second spacer segment comprises an exon. In some embodiments, the second spacer segment further comprises a polyA sequence. In some embodiments, the polyA sequence is 3′ of the exon. In some embodiments, the polyA sequence comprises a rabbit beta globin (RBG) polyA sequence.

In some embodiments, the second spacer segment comprises from 5′ to 3′: a) a first recombination site; b) the first 3′ splice site; c) an exon; d) a stop signaling sequence; and e) a second recombination site. In some embodiments, the first recombination site is a first lox sequence and the second recombination site is a second lox sequence. In some embodiments, the first lox sequence is a first loxP sequence and a second lox sequence is a second loxP sequence. In some embodiments, the first recombination site is a first FRT site and the second recombination site is a second FRT site. In some embodiments, the stop signaling sequence is a termination codon of the exon or a polyA sequence. In some embodiments, the polyA sequence comprises a rabbit beta globin (RBG) polyA sequence. In some embodiments, the exon encodes a detectable marker or a selectable marker. In some embodiments, the detectable marker comprises a luminescent marker or a fluorescent marker. In some embodiments, the fluorescent marker is GFP, EGFP, RFP, CFP, BFP, YFP, or mCherry.

In some embodiments, the second spacer segment is excisable by a recombinase. In some embodiments, the recombinase is a Cre polypeptide or a Flippase polypeptide. In some embodiments, the Cre polypeptide is fused to a ligand binding domain. In some embodiments, the ligand binding domain is a hormone receptor. In some embodiments, the recombinase is a Cre-ERT2 polypeptide.

In some embodiments, the Rep gene codes for Rep polypeptides. In some embodiments, the Cap gene codes for Cap polypeptides. In some embodiments, transcription of the Rep gene and the Cap gene are driven by native promoters. In some embodiments, the native promoters comprise P5, P19, and P40.

In some embodiments, the Rep polypeptides are wildtype Rep polypeptides. In some embodiments, the Rep polypeptides comprise Rep78, Rep68, Rep52, and Rep40. In some embodiments, a truncated replication associated protein comprising a polypeptide expressed from the sequence of first part of a Rep gene and the exon is capable of being expressed in the absence of the recombinase.

In some embodiments, the Cap polypeptides are wildtype Cap polypeptides. In some embodiments, the Cap polypeptides are AAV capsid proteins. In some embodiments, the AAV capsid proteins comprise VP1, VP2, and VP3. In some embodiments, a serotype of the AAV capsid proteins is selected from the group consisting of AAV1, AAV2, AAV3, AAV4, AAV5, AAV6, AAV7, AAV8, AAV9, AAV 10, AAV11, AAV 12, AAV13, AAV 14, AAV 15 and AAV 16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, and AAVhu68.

In some embodiments, the first polynucleotide construct further comprises a sequence coding for a selectable marker. In some embodiments, the selectable marker is a mammalian cell selection element. In some embodiments, the selectable marker is an auxotrophic selection element. In some embodiments, the auxotrophic selection element codes for an active protein. In some embodiments, the active protein is DHFR. In some embodiments, the selectable marker is an antibiotic resistance protein. In some embodiments, the selectable marker is a split intein linked to an N-terminus of the antibiotic resistance protein or split intein linked to a C-terminus of the antibiotic resistance protein. In some embodiments, the selectable marker is a leucine zipper linked to an N-terminus of the antibiotic resistance protein or leucine zipper linked to a C-terminus of the antibiotic resistance protein. In some embodiments, the antibiotic resistance protein is for puromycin resistance or blasticidin resistance.

In some embodiments, the first polynucleotide construct comprises a sequence having at least 70%, 80%, 90%, 95%, 99%, or 100% sequence identity to any one of SEQ ID NO: 1-SEQ ID NO: 3, SEQ ID 6-SEQ ID NO: 8, or SEQ ID NO: 32. In some embodiments, the second polynucleotide construct has at least 70%, 80%, 90%, 95%, 99%, or 100% sequence identity to any one of SEQ ID NO: 9-SEQ ID NO: 19, SEQ ID 23-SEQ ID NO: 32, or SEQ ID NO: 35. In some embodiments, the first polynucleotide construct and the second polynucleotide construct are stably integrated in the cell's genome.

In some embodiments, the cell further comprises a payload construct, wherein the payload construct is a polynucleotide coding for a payload. In some embodiments, the payload construct comprises a sequence having at least 70%, 80%, 90%, 95%, 99%, or 100% sequence identity to SEQ ID NO: 33. In some embodiments, the payload construct comprises a sequence of a payload flanked by ITR sequences. In some embodiments, expression of the sequence of the payload is driven by a constitutive promoter. In some embodiments, the constitutive promoter and sequence of the payload are flanked by ITR sequences.

In some embodiments, the sequence of the payload comprises a polynucleotide sequence coding for a gene. In some embodiments, the gene codes for a selectable marker or detectable marker. In some embodiments, the gene codes for a therapeutic polypeptide or transgene.

In some embodiments, the sequence of the payload comprises a polynucleotide sequence coding for a therapeutic polynucleotide. In some embodiments, the therapeutic polynucleotide is a tRNA suppressor or a guide RNA. In some embodiments, the guide RNA is a polyribonucleotide capable of binding to a protein. In some embodiments, the protein is nuclease. In some embodiments, the protein is a Cas protein, an ADAR protein, or an ADAT protein. In some embodiments, the Cas protein is catalytically inactive Cas protein. In some embodiments, the payload construct is stably integrated into the genome of the cell.

In some embodiments, a plurality of the payload construct are stably integrated into the genome of the cell. In some embodiments, the plurality of the payload constructs are separately stably integrated into the genome of the cell. In some embodiments, the payload construct further comprises a sequence coding for a selectable marker or detectable marker outside of the ITR sequences. In some embodiments, the payload construct is integrated into the genome of the cell.

Also provided herein are methods of producing a stable cell line comprising expanding a cell described above.

Also provided herein are methods of producing a plurality of rAAV virion comprising culturing a cell described above in the presence of a first triggering agent and a second triggering agent. In some embodiments, the first triggering agent is doxycycline and the second triggering agent is tamoxifen. In some embodiments, the plurality of rAAV virion have an encapsidation ratio of no less than 0.5, 0.6, 0.7, 0.8, 0.9, 0.95, 0.97, or 0.99 prior to purification. In some embodiments, the plurality of rAAV virion have a F:E ratio of no less than 0.5, 0.6, 0.7, 0.8, 0.9, 0.95, 0.97, or 0.99 prior to purification. In some embodiments, the plurality of rAAV virion have a concentration of greater than 1×10¹¹ or no less than 5×10¹¹, 1×10¹², 5×10¹², 1×10¹³ or 1×10¹⁴ viral genomes per milliliter prior to purification. In some embodiments, the plurality of rAAV virion have an infectivity of no less than 50%, 60%, 70%, 80%, 90%, 95%, or 99% at an MOI of 1×10⁵ vg/target cell or less. In some embodiments, the culturing is in a bioreactor.

Also provided herein are pharmaceutical compositions comprising the rAAV virion produced by the cell or the method described above and a pharmaceutically acceptable carrier. Also provided herein are methods of treating a condition or disorder, the method comprising administering a therapeutically effective amount of the pharmaceutical composition to a patient in need thereof.

### 5. BRIEF DESCRIPTION OF THE DRAWINGS

FIG. 1 depicts the pre-triggered state of an exemplary cell in which a plurality of synthetic nucleic acid constructs have been separately integrated into the nuclear genome. This exemplary cell gives rise to a stable cell line capable of conditionally producing recombinant AAV (rAAV) virions that package a payload (e.g., a therapeutic polynucleotide). The brackets in construct 3 indicate the position of the flanking ITRs.

FIGS. 2A-2C depict an exemplary embodiment of construct 2 from FIG. 1 in greater detail. This construct permits conditional expression of Cre. In the pre-triggered state (top of FIG. 2A) the integrated nucleic acid construct has a Cre coding sequence and adenoviral E2A and E4 helper protein coding sequences collectively under the control of an inducible promoter that becomes active upon the addition of a triggering agent. Other coding elements (activator and a gene that permits mammalian selection (mammalian selection)) are under control of a constitutive promoter (“CMV promoter”). The Cre coding element is positioned between LoxP sites and is additionally fused to estrogen response elements (“ER2”), which allows for control over the localization of Cre in response to estrogen agonists, such as tamoxifen. Upon addition of a triggering agent, Cre is expressed (bottom of FIG. 2A), and upon addition of Tamoxifen, Cre translocates to the nucleus. As shown in FIG. 2B, following translation and translocation of the Cre protein into the cell nucleus, the Cre protein effects excision of its own coding sequence, leaving the integrated construct shown at the bottom of FIG. 2B. Shown in FIG. 2C is an optional insert in construct 2. The optional insert includes a Cre inducible U6 promoter that drives the expression of transcriptionally dead mutants of VA RNA1 (VA RNA). Specifically, the U6 promoter is split into two parts separated by a Lox flanked stuffer sequence. The U6 promoter is inactive because of the presence of the stuffer sequence. Cre mediated excision of the stuffer sequence activates the U6 promoter, which then drives the expression of VA RNA.

FIGS. 3A-3B are schematics depicting details of an exemplary embodiment of construct 1. This construct is designed to permit expression of AAV Rep and Cap proteins from their endogenous promoters after a triggering event. FIG. 3A shows the pre-triggered state of the integrated nucleic acid construct. An intervening spacer (excisable spacer) interrupts the Rep coding sequence. The excisable spacer comprises a first spacer segment, a second spacer segment which is excisable (second “excisable” spacer segment), and a third spacer segment. The second “excisable” spacer segment comprises EGFP flanked by LoxP sites and an upstream 3′ splice site (3′SS). A pre-triggered transcript is shown at the bottom of the figure. This pre-triggered transcript encodes the 5′ portion of AAV rep fused to a fluorescent marker protein, EGFP. The pre-triggered transcript contains a single intron flanked by 5′ splice site (5′SS) and 3′ splice site (3′SS). FIG. 3B shows the conversion of the pre-triggered construct (top schematics) to a post-triggered state (bottom schematics) upon exposure to Cre in the cell nucleus. Cre excises the second “excisable” spacer segment, which includes the EGFP marker coding sequence and the upstream 3′ splice site (3′ SS). When the second “excisable” spacer segment is excised by Cre, the construct allows for expression of functional Rep and Cap transcripts from their respective endogenous promoters.

FIG. 4 depicts an exemplary embodiment of construct 3. Construct 3 comprises a sequence that encodes a payload (payload polynucleotide). The payload polynucleotide is under control of a constitutive promoter. The brackets indicate the position of the flanking ITRs. As indicated, in various nonlimiting embodiments, the payload is a transgene encoding a protein of interest, a homology element for homology-directed repair (e.g., HDR homology region), or a guide RNA. Also shown is a coding sequence coding for a protein that permits selection in mammalian cells (mammalian selection).

FIG. 5A depicts an exemplary embodiment of a split auxotrophic selection system that permits stable retention of two integrated nucleic acid constructs under a single selective pressure. One construct encodes the N-terminal fragment of mammalian dihydrofolate reductase (DHFR) fused to a leucine zipper peptide (“Nter-DHFR”). This N-terminal fragment is enzymatically nonfunctional. The other construct encodes the C-terminal fragment of DHFR fused to a leucine zipper peptide (“Cter-DHFR”). This C-terminal fragment is enzymatically nonfunctional. When both fragments are concurrently expressed in the cell, a functional DHFR enzyme complex is formed through association of the leucine zipper peptides. Both constructs can be stably retained in the genome of a DHFR null cell by growth in a medium lacking hypoxanthine and thymidine (-HT selection).

FIG. 5B shows an exemplary deployment of this split auxotrophic selection design in the multi-construct system of FIG. 1 in its pre-triggered state. In this example, the split auxotrophic selection elements are deployed in constructs 1 and 3. A separate exemplary antibiotic selection approach, blasticidin resistance, is deployed in construct 2. This results in the ability to stably maintain all three constructs in the mammalian cell line by culturing in medium having a single antibiotic, blasticidin, and lacking both thymidine and hypoxanthine.

FIG. 6 depicts the post-triggered state of all 3 constructs following the addition of tamoxifen and a triggering agent. In the presence of the triggering agent, adenoviral E2A and E4 helper proteins are expressed. AAV rep and cap coding sequences are expressed under control of endogenous promoters. The payload is expressed under control of a constitutive promoter. rAAV virions encapsidating the payload are therefore produced. The three integrated constructs are stably maintained in the nuclear genome with a single antibiotic (Blasticidin) and auxotrophic selection (media lacking both thymidine and hypoxanthine).

FIGS. 7A-7B are light and fluorescence microscopic images. Light microscope images are presented in the left column. Green fluorescence images are presented in the middle column. Red fluorescence images are presented in the right column. Following addition of different amounts of Cre vesicles containing Cre protein and a red fluorescence marker protein, cells were either mock-transfected (“Mock”), transfected with a plasmid having construct 1 (“AAV2 CODE”), or transfected with a control AAV2 plasmid capable of expression Rep and Cap proteins (“AAV2”). Without addition of Cre (FIG. 7A), only the cells transfected with a plasmid having Construct 1 show intense green fluorescence, indicating expression of the Rep-EGFP fusion protein (see bottom of FIG. 3A). FIG. 7B shows decreased EGFP fluorescence in the presence of increasing amounts of Cre, indicating recombination and subsequent removal of EGFP cassette from construct 1.

FIGS. 8A-8B are blots and graphs showing Rep production from post-triggered plasmid construct 1. FIG. 8A shows Western blots illustrating that Cre-mediated excision of the excisable spacer segment induces Rep protein production from post-triggered plasmid construct 1. In addition, the presence of the rabbit beta globin intron does not interfere with Rep protein expression level. FIG. 8B shows a schematic of a Rep/Cap polynucleotide construct, which is cloned into a piggybac vector with a Blasticidin resistance gene (SEQ ID NO: 8). The excisable element interrupting the Rep gene was inserted downstream of the p19 promoter. The GFP levels confirm successful integration of the Rep/Cap construct in cells from STXC0068 cell line (bottom FACS plot) compared to cells from the parental cell line (top FACS plot). Graphs of the cell density (top graph) and viability (bottom graph) data of the STXC0068 cell line illustrate that there were no negative effects from the integrated AAV sequences. The left blot shows the production of Rep proteins and the right blot shows total protein, for the parental cell line, the parental cell line after the addition of Cre, the STXC0068 cell line, and the STXC0068 cell line after the addition of Cre.

FIG. 9A presents a schematic of the PKR pathway interactions (SEQ ID NOs: 40 and 41).

FIG. 9B shows a diagram VA RNA construct and the sequence of VA RNA1 that shows its double-stranded RNA (dsRNA) structure.

FIGS. 10A-10B depict the plasmid descriptions (FIG. 10A) and testing (FIG. 10B) of these plasmid constructs comprising VA RNA constructs including wildtype VA RNA (pHelper), VA RNA knockout (STXC002), and VA RNA knockout with a compensatory viral protein (infected cell protein 34.5 (ICP34.5); STXC0016) to evaluate the effect of VA RNA on AAV titers.

FIGS. 11A-11B depict the plasmid descriptions (FIG. 11A) and testing of these plasmids (FIG. 11B) comprising VA RNA promoter mutants for the relative VA RNA expression in adherent HEK293 cells.

FIGS. 12A-12D depict the design of an inducible U6 promoter segment containing mutant VA RNA (FIG. 12A), the plasmid descriptions (FIG. 12B) for the control and test plasmids, and the relative VA RNA expression in LV max cells (FIGS. 12C & 12D), illustrating rescue of select mutants with an inducible promoter.

FIGS. 13A-13B are graphs showing titer results using the mutant and inducible VA RNA constructs from FIG. 12B in HEK293T cells (FIG. 13A) and LV Max cells (FIG. 13B).

FIG. 14 shows a plasmid map of STX_C002, which is a helper plasmid without VA RNA expression (VA RNA is deleted).

FIG. 15 shows a plasmid map of STX_C0032, which is a STX_C002helper plasmid backbone containing a WT VA RNA.

FIG. 16 shows a plasmid map of STX_C0033, which is a STX_C002 helper plasmid backbone containing a VA RNA1 B1 mutant (a six-nucleotide segment deleted from the B Box) with the VA RNA in reverse orientation.

FIG. 17 shows a plasmid map of STX_C0036, which is a STX_C002 helper plasmid containing the VA RNA mutations G16A and T45C, with the VA RNA in reverse orientation).

FIG. 18 shows a plasmid map of STX_C0041, which is made by modifying the STX_00033 helper construct containing VA RNA1 B1 mutant (a six nucleotide segment deleted from the B Box) to contain a U6 inducible promoter construct (as shown in FIG. 12A). The position of the new U6 promoter and Lox sites are shown.

FIG. 19 shows a plasmid map of STX_C0042 which is made by modifying the STX_C0035 helper plasmid containing the VA RNA mutations G16A and T45C to contain a U6 inducible promoter construct (as shown in FIG. 12A). The position of the new U6 promoter and Lox sites are shown.

FIG. 20 shows a plasmid map of STX_C0043 which is made by modifying the STX_C0037 helper plasmid containing the VA RNA mutations G16A and G60A. The position of the new U6 promoter and Lox sites are shown.

FIG. 21 shows a plasmid map of STX_C0037 containing the VA RNA mutations G16A and G60A.

FIG. 22 is a schematic showing the production of an exemplary stable cell line (P2 Producer Cell Line) containing a Rep/Cap construct, an inducible helper construct, and a construct with payload construct and the packaging of the payload (Gene of Interest) into virions. The P1 Helper Cell Line is produced from integration of either inducible helper construct #1 or inducible construct #2 into the Serum Free Suspension Adapted 293 cells.

FIG. 23 shows plasmid maps and a graph showing Rep production using inducible bicistronic constructs in a transient transfection system.

FIG. 24 shows schematics of STXC0090 and STXC0110 constructs illustrating helper and Cre induction using the TetOn system.

FIG. 25 shows schematics of VA RNA mutant constructs and various promoter and selection options.

FIG. 26 shows intracellular staining for expression of FLAG-tagged E2A from cells with stable integration of STXC-0123 (T33, left plot), STXC-0124 (T34, middle plot), or STXC-0125 (T35, right plot) helper constructs, and after either mock induction, no induction, or induction of Cre.

FIG. 27 shows an overview of HEK293 cells with the stably integrated helper plasmid showing no cytotoxic effects and induction of Cre, production of VA RNA and good distribution of E2A expression.

FIG. 28 shows work flows for producing stable cell line pools. The schematic and work flow on the left illustrates integration of STXC0123 to produce the T33 pool, in which STXC0137 and STXC0136 are then integrated, to produce three stable cell line pools (T40, T41, and T42). The schematic and work flow on the right illustrates integration of STXC0133 to produce the T44 pool, in which STXC0137 and STXC0136 are then integrated, to produce three stable cell line pools (T56, T57, and T58). The stable cell line pools are then treated with doxycycline and tamoxifen to produce virions encapsidating STXC650.

FIG. 29 shows graphs of the viable cell density (left graph) and viability (right graph) for the T33 pool and T44 pool (FIG. 28), and illustrate that there were no negative effects from the integrated plasmid constructs.

FIG. 30 shows graphs for E2A expression, VA RNA expression, culture density and culture viability for the T33 pool stable cell line, the T44 stable cell line, and the parental cell line (VPC) either not induced or after induction. The left graph is at 24 hours post induction and the right graph is at 48 hours post induction.

FIG. 31 shows graphs of the viable cell density (left graph) and viability (right graph) for the T40, T41, and T42 cell line pools illustrated in FIG. 28.

FIG. 32 shows graphs of the viable cell density (right graph) and viability (left graph) for the T59, T60, and T61 cell line pools, which were produced the same way as the T56, T57, and T58 cell line pools illustrated in FIG. 28.

FIG. 33 shows a graph of capsid production from the T42 pool stable cell line after induction compared to cells produced by transient triple transfection (3×Tfxn) in various cell medias (AAV, Bal, Cyt 2, Cyt9, Fuji 7, Fuji 7-2, HE300, TS1, TS3, or TS5). The left bar for each media type indicates total capsid titer and the right bar for each media type indicates the titer of capsids encapsidating a viral genome (e.g., the payload construct).

FIG. 34 shows the titer of capsids encapsidating a viral genome (e.g., the payload construct) for the T42 stable cell line pool, T59 stable cell line pool, T60 stable cell line pool, and T61 stable cell line pool either with (+) or without (−) induction in HE300 media.

FIG. 35 shows infectivity as indicated by the percentage of GFP+ cells after infecting target cells (CHO Pro-5 cells) with capsids from the T42 pool stable cell line compared to the T61 pool stable cell line in various media. The left bar for each cell line type/media is for a dilution factor of 1 and the right bar for each cell line type/media is for a dilution factor of 4.

FIG. 36 shows a graph of the titer of capsids encapsidating a viral genome (e.g., the payload construct) per cell from the T42 pool stable cell line after induction compared the titer of capsids encapsidating a viral genome (e.g., the payload construct) to per cell from the triple transfected parental cells (VPC) in various cell medias. The left bar for each media type indicates titer of capsids encapsidating a viral genome produced per cell from the T42 pool stable cell line and the right bar for each media type indicates titer of capsids encapsidating a viral genome produced per cell from the triple transfected parental cell line (VPC).

FIG. 37 shows a graph of the dilution adjusted titer of capsids encapsidating a viral genome (e.g., the payload construct) from the T42 pool stable cell line after induction at different seed densities in various cell medias. The 3×Tfxn dashed line indicates the dilution adjusted titer of capsids encapsidating a viral genome (e.g., the payload construct) produced by cells after transient triple transfection (3×Tfxn).

FIG. 38 shows a graph of total capsids in different cell media with mini pool clones selected from the T42 pool stable cell line compared to the T42 pool stable cell line after induction in different cell media.

FIG. 39 shows infectivity as indicated by the percentage of GFP+ cells (encapsidated payload) after infecting target cells (CHO Pro-5 cells) with capsids versus multiplicity of infection (vg/cell) for mini pool clones selected from the T42 pool stable cell line in various cell media from FIG. 38. (Control is a purified capsid produced by cells after transient transfection).

FIG. 40 shows infectivity as indicated by the percentage of GFP+ cells (encapsidated payload) after infecting target cells (CHO Pro-5 cells) with capsids versus multiplicity of infection (vg/cell) for mini pool clones selected from the T42 pool stable cell line in various cell media from FIG. 38. The inset control shows infectivity as indicated by the percentage of GFP+ cells (encapsidated payload) after infecting target cells (CHO Pro-5 cells) with capsids versus multiplicity of infection (vg/cell) for a purified capsid produced by cells after transient transfection.

### 6. DETAILED DESCRIPTION

To solve the problems presented by transient transfection approaches to rAAV production while addressing the toxicity of AAV Rep protein when constitutively expressed, disclosed herein are polynucleotide constructs and cell lines stably integrated with said polynucleotide constructs (referred to herein as “stable cell lines”) that enable conditional (also referred to herein as “inducible”) production of recombinant AAV (rAAV) virions. In some embodiments, the compositions and methods of use thereof as disclosed herein provide rAAV virions that encapsidate a desired expressible payload, such as an expressible therapeutic payload. Further provided herein is a stable mammalian cell line, wherein the cells are capable of conditionally producing recombinant AAV (rAAV) virions within which are packaged an expressible payload; and wherein a population of virions produced by the stable cell are more homogenous than a population of virions produced by an otherwise comparable cell producing rAAV virions upon transient transfection.

Further provided herein is a stable mammalian cell line, wherein the cells are capable of conditionally producing recombinant AAV (rAAV) virions within which are packaged an expressible payload; and production of virions is inducible upon addition of a triggering agent.

Further provided herein is a stable mammalian cell line, wherein the cells are capable of conditionally producing recombinant AAV (rAAV) virions within which are packaged an expressible payload; and production of virions is not conditioned on the presence of a plasmid within the cell.

### 6.1. DEFINITIONS

Unless defined otherwise, all technical and scientific terms used herein have the meaning commonly understood by one of ordinary skill in the art to which the invention pertains.

“Recombinant”, as applied to an AAV virion, means that the rAAV virion (synonymously, rAAV virus particle) is the product of one or more procedures that result in an AAV particle construct that is distinct from an AAV virion in nature.

In some aspects, the disclosure provides transfected host cells. The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

A “host cell” refers to any cell that harbors, or is capable of harboring, a substance of interest. Often a host cell is a mammalian cell. A host cell may be used as a recipient of an AAV helper construct, an AAV minigene plasmid, an accessory function vector, or other transfer DNA associated with the production of recombinant AAVs. The term includes the progeny of the original cell which has been transfected. Thus, a “host cell” may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation. A “host cell” as used herein may refer to any mammalian cell which is capable of functioning as an adenovirus packaging cell, i.e., expresses any adenovirus proteins essential to the production of AAV, such as HEK 293 cells and their derivatives (HEK293T cells, HEK293F cells), HeLa, A549, Vero, CHO cells or CHO-derived cells, and other packaging cells.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

The term “cell culture,” refers to cells grown adherent or in suspension, bioreactors, roller bottles, hyperstacks, microspheres, macrospheres, flasks and the like, as well as the components of the supernatant or suspension itself, including but not limited to rAAV particles, cells, cell debris, cellular contaminants, colloidal particles, biomolecules, host cell proteins, nucleic acids, and lipids, and flocculants. Large scale approaches, such as bioreactors, including suspension cultures and adherent cells growing attached to microcarriers or macrocarriers in stirred bioreactors, are also encompassed by the term “cell culture.” Cell culture procedures for both large and small-scale production of proteins are encompassed by the present disclosure.

As used herein, the term “intermediate cell line” refers to a cell line that contains the AAV rep and cap components integrated into the host cell genome or a cell line that contains the adenoviral helper functions integrated into the host cell genome.

As used herein, the term “packaging cell line” refers to a cell line that contains the AAV rep and cap components and the adenoviral helper functions integrated into the host cell genome. A payload construct must be added to the packaging cell line to generate rAAV virions.

As used herein, the term “production cell line” refers to a cell line that contains the AAV rep and cap components, the adenoviral helper functions, and a payload construct. The rep and cap components and the adenoviral helper functions are integrated into the host cell genome. The payload construct can be stably integrated into the host cell genome or transiently transfected. rAAV virions can be generated from the production cell line upon the introduction of one or more triggering agents in the absence of any plasmid or transfection agent.

As used herein, the term “downstream purification” refers to the process of separating rAAV virions from cellular and other impurities. Downstream purification processes include chromatography-based purification processes, such as ion exchange (IEX) chromatography and affinity chromatography.

The term “prepurification yield” refers to the rAAV yield prior to the downstream purification processes. The term “postpurification yield” refers to the rAAV yield after the downstream purification processes. rAAV yield can be measured as viral genome (vg)/L.

The encapsidation ratio of a population of rAAV virions can be measured as the ratio of rAAV viral particle (VP) to viral genome (VG). The rAAV viral particle includes empty capsids, partially full capsids (e.g., comprising a partial viral genome), and full capsids (e.g., comprising a full viral genome).

The F:E ratio of a population of rAAV virions can be measured as the ratio of rAAV full capsids to empty capsids. The rAAV full capsid particle includes partially full capsids (e.g., comprising a partial viral genome) and full capsids (e.g., comprising a full viral genome). The empty capsids lack a viral genome.

The potency or infectivity of a population of rAAV virions can be measured as the percentage of target cells infected by the rAAV virions at a multiplicity of infection (MOI; viral genomes/target cell). Exemplary MOI values are 1×101, 1×102, 2×103, 5×104, or 1×105 vg/target cell. An MOI can be a value chosen from the range of 1×10¹ to 1×10⁵ vg/target cell.

As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. Thus, the term includes cloning and expression vehicles, as well as viral vectors. The use of the term “vector” throughout this specification refers to either plasmid or viral vectors, which permit the desired components to be transferred to the host cell via transfection or infection. For example, an adeno-associated viral (AAV) vector is a plasmid comprising a recombinant AAV genome. In some embodiments, useful vectors are contemplated to be those vectors in which the nucleic acid segment to be transcribed is positioned under the transcriptional control of a promoter.

The phrases “operatively positioned,” “operatively linked,” “under control” or “under transcriptional control” means that the promoter is in the correct location and orientation in relation to the nucleic acid to control RNA polymerase initiation and expression of the gene.

The term “expression vector or construct” or “synthetic construct” means any type of genetic construct containing a nucleic acid in which part or all of the nucleic acid encoding sequence is capable of being transcribed. In some embodiments, expression includes transcription of the nucleic acid, for example, to generate a biologically-active polypeptide product or functional RNA (e.g., guide RNA) from a transcribed gene.

The term “auxotrophic” or “auxotrophic selection marker” as used herein refers to the usage of a medium lacking a supplement, such as a medium lacking an essential nutrient such as the purine precursors hypoxanthine and thymidine (HT), or the like, for selection of a functional enzyme which allows for growth in the medium lacking the essential nutrient, e.g. a functional dihydrofolate reductase or the like.

The term cytostatic as used herein refers to a cellular component or agent/element or condition that inhibits cell growth. Cytostasis is the inhibition of cell growth and multiplication.

The term cytotoxic as used herein refers to quality of being toxic to cells. For instance, cells exposed to a cytotoxic agent or condition may undergo necrosis, in which they lose membrane integrity and die rapidly as a result of cell lysis. Cells exposed to a cytotoxic agent can also stop actively growing and dividing (a decrease in cell viability), or the cells can activate a genetic program of controlled cell death (apoptosis).

As used herein, a “monoclonal cell line” or “monoclonality” is used to describe cells produced from a single ancestral cell by repeated cellular replication. Thus, “monoclonal cells” can be said to form a single clone.

The terms “tetracycline” is used generically herein to refer to all antibiotics that are structurally and functionally related to tetracycline, including tetracycline, doxycycline, demeclocycline, minocycline, sarecycline, oxytetracycline, omadacycline, or eravacycline.

The terms “constitutive” or “constitutive expression” are used interchangeably herein. They refer to genes that are transcribed in an ongoing manner. In some embodiments, the terms refer to the expression of a therapeutic payload or a nucleic acid sequence that is not conditioned on addition of an expression triggering agent to the cell culture medium.

The term “expressible therapeutic polynucleotide or “expressible polynucleotide encoding a payload” or “payload polynucleotide” or “payload” refers to a polynucleotide that is encoded in an AAV genome vector (“AAV genome vector”) flanked by AAV inverted terminal repeats (ITRs). A payload disclosed herein may be a therapeutic payload. A payload may include any one or combination of the following: a transgene, a tRNA suppressor, a guide RNA, or any other target binding/modifying oligonucleotide or derivative thereof, or payloads may include immunogens for vaccines, and elements for any gene editing machinery (DNA or RNA editing). Payloads can also include those that deliver a transgene encoding antibody chains or fragments that are amenable to viral vector-mediated expression (also referred to as “vectored or vectorized antibody” for gene delivery). See, e.g. Curr Opin HIV AIDS. 2015 May; 10(3): 190-197, describing vectored antibody gene delivery for the prevention or treatment of HIV infection. See also, U.S. Pat. No. 10,780,182, which describes AAV delivery of trastuzumab (Herceptin) for treatment of HER2+ brain metastases. A payload disclosed herein may not be a therapeutic payload (e.g., a coding for a detectable marker such as GFP).

In particular, in some instances the payload polynucleotide refers to a polynucleotide that can be a homology element for homology-directed repair, or a guide RNA to be delivered for a variety of purposes. In some embodiments, the transgene refers to a nucleic acid sequence coded for expression of guide RNA for ADAR editing or ADAT editing. In some embodiments, the transgene refers to a transgene packaged for gene therapy. In some embodiments, the transgene refers to synthetic constructs packaged for vaccines.

### 6.2. System Overview

The stable mammalian cell line relies on stable integration and maintenance of a plurality of synthetic nucleic acid constructs within the nuclear genome of the cell. One of these constructs permits inducible expression of a hormone-activated excising element. The excising element can be a recombinase. The recombinase can be a site-specific recombinase. The site-specific recombinase can be a Cre polypeptide or a flippase. Triggering of Cre expression leads to genomic rearrangements, which in turn lead to expression of adenovirus helper proteins, expression of AAV Rep and Cap proteins, and production of rAAV, optionally, encapsidating a therapeutic payload (e.g., transgene, a tRNA suppressor, a guide RNA, or other oligonucleotide).

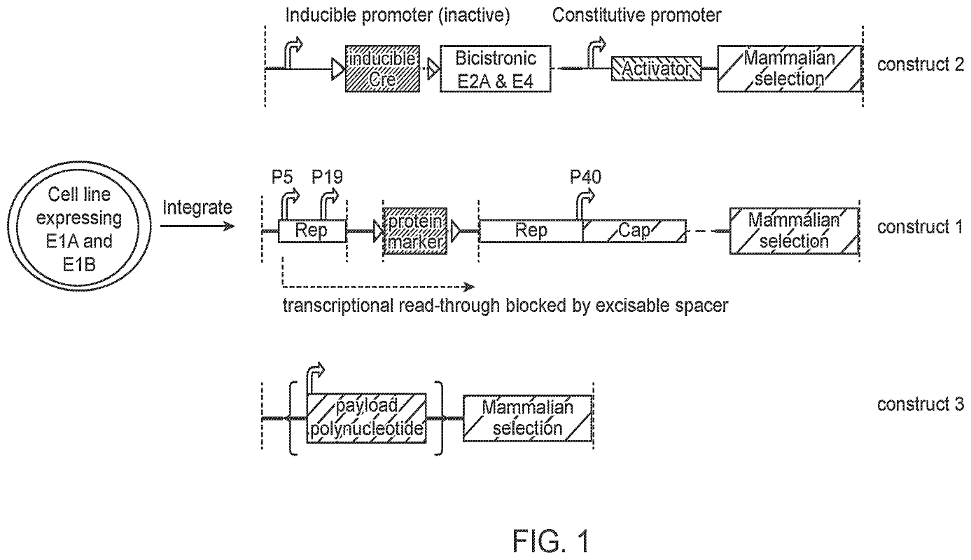

FIG. 1 depicts the pre-triggered state of an exemplary embodiment. In the embodiment shown, three synthetic nucleic acid constructs are separately integrated into the nuclear genome of a cell line that expresses adenovirus E1A and E1B, such as HEK 293 cells. In the pre-triggered state, transcriptional read-through of rep on construct 1 is blocked by an intervening spacer. The payload polynucleotide on construct 3 is flanked by AAV ITRs, represented by the brackets.

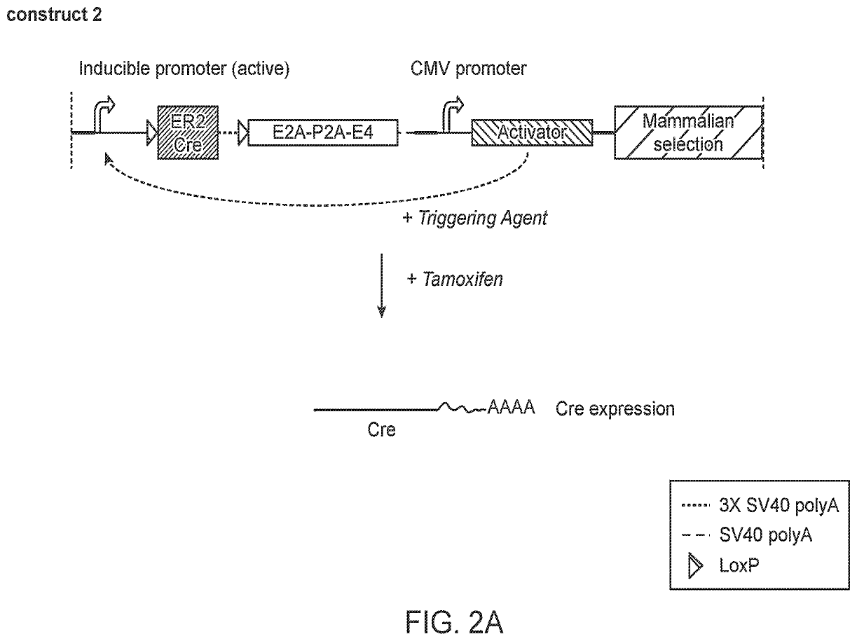

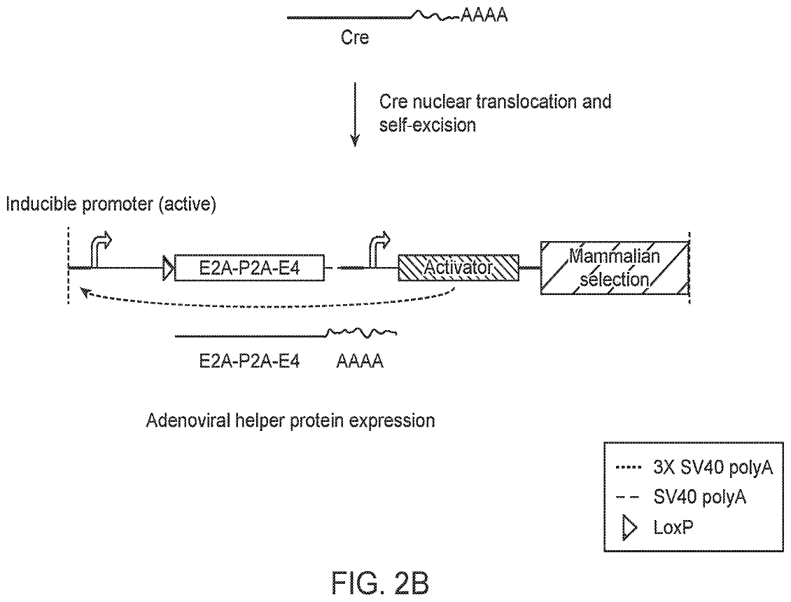

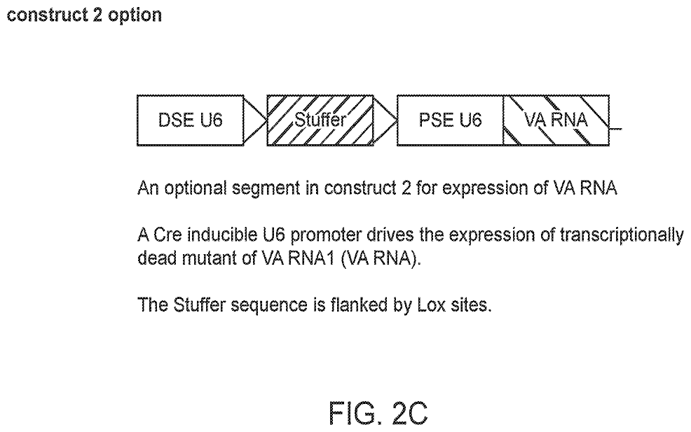

An exemplary construct 2 is shown in greater detail in FIGS. 2A-2C. This construct permits conditional expression of Cre. In some embodiments, the construct 2 comprises a P2A sequence positioned between an E2A sequence and an E4 sequence. In some embodiments, the construct 2 comprises an internal ribosomal entry site (IRES) sequence positioned between an E2A sequence and an E4 sequence. In some embodiments, the inducible promoter system of construct 2 is a Tet On inducible promoter system. In some embodiments, the inducible promoter system of construct 2 is a Tet Off inducible promoter system. In some embodiments, the inducible promoter system of construct 2 is a cumate inducible promoter system.

In the pre-triggered state (top of FIG. 2A), the Cre coding sequence is under the control of an inducible promoter. For example, the inducible promoter is a Tet-inducible promoter. In the absence of a triggering agent, inducible promoter is not active. For example, a triggering agent for Tet-inducible promoter is a tetracycline. In the absence of a tetracycline, such as doxycycline (“Dox”), Tet activator protein (TetOn3G) cannot bind and activate the basal Tet On promoter. In addition, the localization of Cre is under control of estrogen response elements (“ER2”) that require binding of an estrogen agonist or selective modulator, such as tamoxifen, for the translocation from the cytoplasm to the nucleus. This approach limits pre-triggering Cre expression with consequent promiscuous recombination events and toxicity. The ER2 Cre element also comprises a strong 3′ polyadenylation signal, which prevents basal expression of the downstream adenoviral helper genes, E2A and E4. In some embodiments, the Cre is split into two fragments, that can be fused in the presence of a chemical agent, such as rapamycin. In some embodiments, the Cre is a light inducible Cre.

When the triggering agent (e.g., Dox) and tamoxifen are added to the culture medium, TetOn3G binds the Tet responsive basal promoter and estrogen response elements are activated, triggering Cre expression (bottom of FIG. 2A). Following translation and then translocation of the Cre protein into the cell nucleus, Cre excises its own coding sequence from construct 2, leaving the integrated construct shown at the bottom of FIG. 2B. Elimination of the upstream poly-adenylation site allows expression of E2A and E4 helper proteins, maintained by the presence of doxycycline. Similarly, for the optional additional insert shown in FIG. 2C, VA-RNA is expressed by Cre mediated excision of the stuffer sequence, which activates the U6 promoter which then drives the expression of VA RNA.

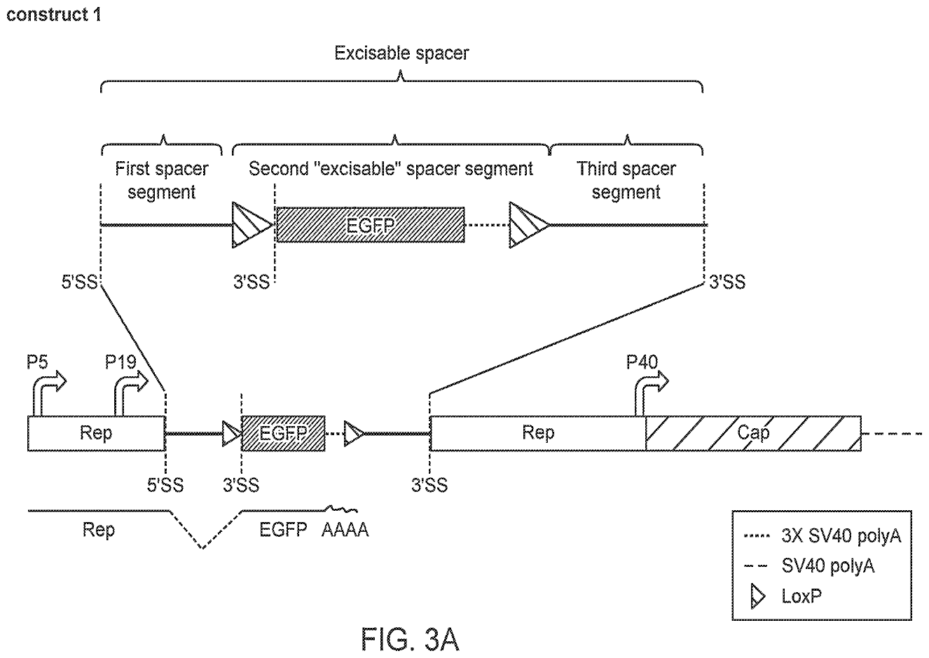

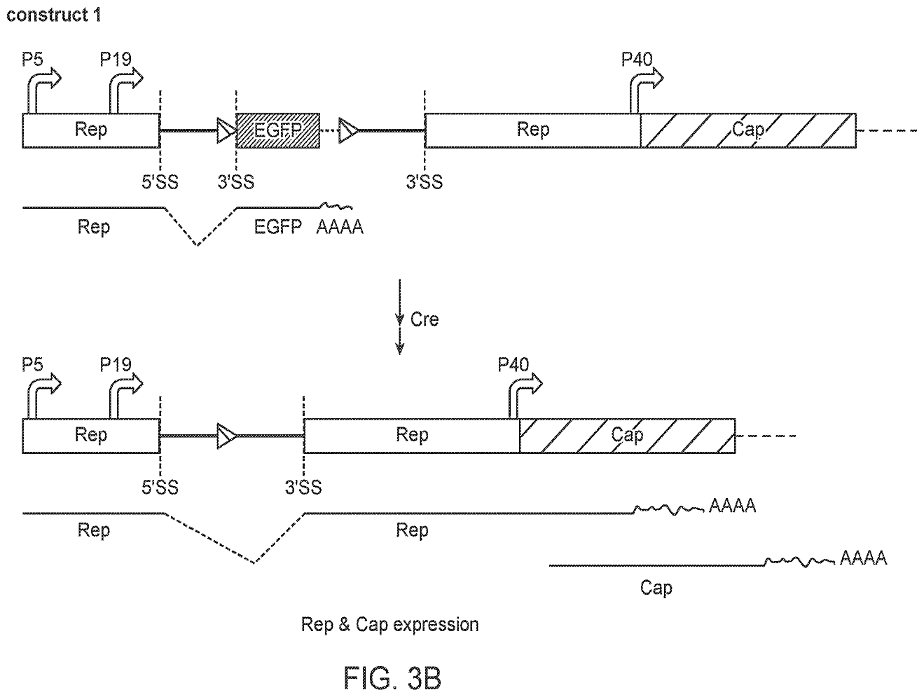

FIGS. 3A-3B schematically depict details of an exemplary embodiment of construct 1. This construct is designed to prevent expression of AAV Rep prior to a triggering event, yet permit expression of AAV Rep and Cap proteins from their endogenous promoters after a triggering event.

FIG. 3A shows the pre-triggered state of integrated nucleic acid construct 1. An excisable spacer interrupts the rep coding sequence, blocking transcriptional read-through of the full-length rep coding sequence. A pre-triggered transcript is shown at the bottom of the figure. This pre-triggered transcript encodes the 5′ portion of AAV Rep fused to a fluorescent marker protein, EGFP. The transcript contains a single intron flanked by 5′ and 3′ splice sites. Routine splicing produces a transcript that encodes a fusion protein that includes the N-terminal portion of rep fused to an enhanced green fluorescent protein (EGFP). The fusion protein lacks the toxicity of full-length Rep protein, and presence of pre-triggered construct 1 in the cell genome can be detected by EGFP fluorescence for quality control. In some embodiments, the EGFP fluorescence is used to select for cells that have integrated nucleic acid construct 1, which then form a stable cell pool. A stable cell pool with the integrated nucleic acid construct 1 can therefore be produced from selecting for cells expressing EGFP.

As shown at the top of FIG. 3A, the excisable spacer comprises a first spacer segment, a second spacer segment, and a third spacer segment. FIG. 3B shows the conversion of the pre-triggered construct (above) to a post-triggered state (below) upon exposure to Cre within the cell nucleus. Cre excises the second spacer segment, which includes the EGFP marker coding sequence and the upstream 3′ splice site. As rearranged, the construct now allows expression of functional Rep and Cap transcripts from their respective endogenous promoters, as shown at the bottom of FIG. 3B. Loss of EGFP expression indicates successful Cre-mediated genomic recombination.

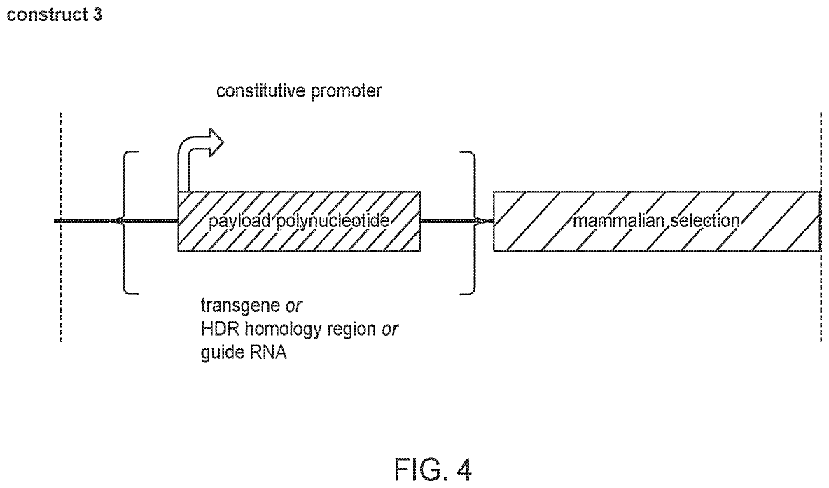

FIG. 4 depicts an exemplary embodiment of construct 3, which is an exemplary payload construct. Construct 3 comprises a sequence that encodes a payload. This sequence element is under control of a constitutive promoter. The payload can be any payload for which rAAV is an appropriate vehicle, including a transgene encoding a protein of interest, a homology element for homology-directed repair, or a guide RNA. The payload is flanked by AAV ITRs, represented by the brackets.

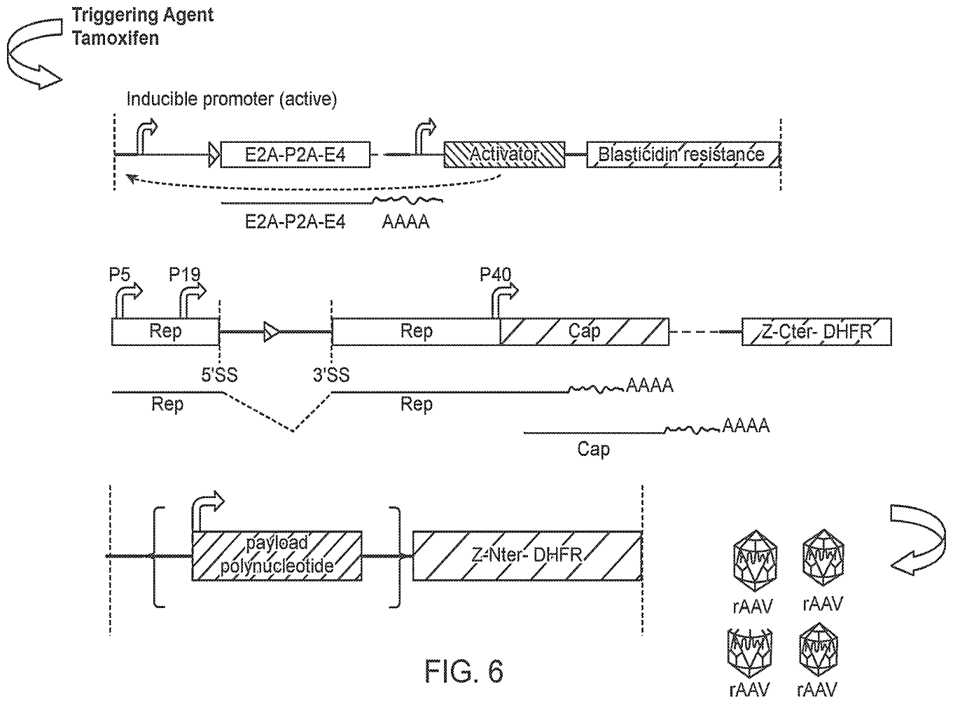

FIG. 6 depicts the post-triggered state of all 3 constructs following the addition of tamoxifen and doxycycline to the cell medium. Adenoviral E2A and E4 helper proteins are expressed from integrated construct 2 under control of the inducible promoter (e.g., a Tet-On promoter activated in the presence of Dox). AAV rep and cap coding sequences are expressed from construct 1 under control of endogenous promoters. The payload is expressed under control of a constitutive promoter. rAAV virions that encapsidate the payload are therefore produced.

This approach provides numerous benefits over current AAV systems for delivery of payloads.

Maintaining constructs stably in the cellular genome requires selective pressure. To reduce the number of selective agents (and in particular, antibiotics) required to stably maintain three integrated constructs within the cell line genome, we have designed an approach that stably maintains all 3 constructs in the nuclear genome with a single antibiotic selection, plus a single auxotrophic selection.

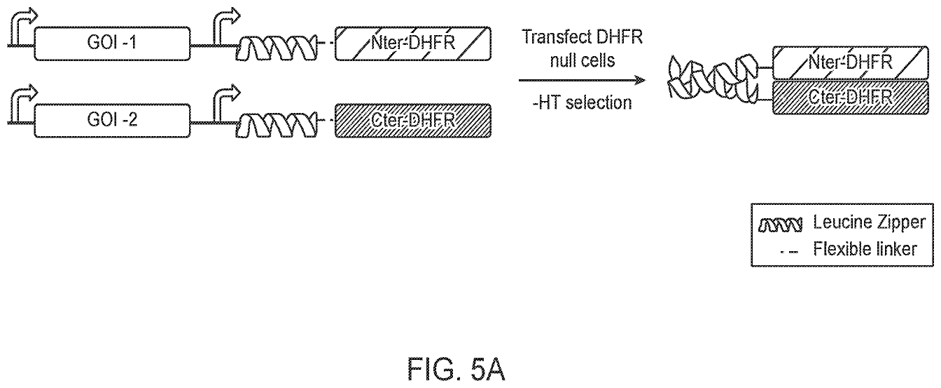

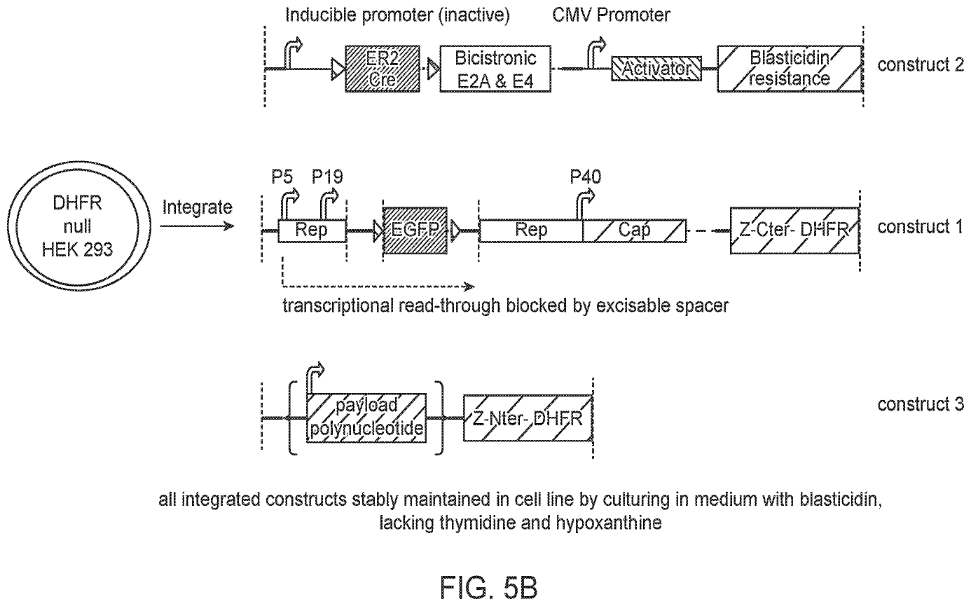

FIG. 5A depicts a split auxotrophic selection system that permits stable retention of two integrated nucleic acid constructs under a single selective pressure. One construct encodes the N-terminal fragment of mammalian dihydrofolate reductase (DHFR) fused to a leucine zipper peptide (“Nter-DHFR”). This N-terminal fragment is enzymatically nonfunctional. The other construct encodes the C-terminal fragment of DHFR fused to a leucine zipper peptide (“Cter-DHFR”). This C-terminal fragment is enzymatically nonfunctional. When both fragments are concurrently expressed in the cell, a functional DHFR enzyme complex is formed through association of the leucine zipper peptides. Both constructs can be stably retained in the genome of a DHFR null cell by growth in a medium lacking hypoxanthine and thymidine.

FIG. 5B shows an exemplary deployment of this split auxotrophic selection design in the multi-construct system of FIG. 1 in its pre-triggered state. In this example, the split auxotrophic selection elements are deployed on constructs 1 and 3. A separate exemplary antibiotic selection approach, blasticidin resistance, is deployed on construct 2. This results in the ability to stably maintain all three constructs in the mammalian cell line using a single antibiotic, culturing in medium with blasticidin, lacking thymidine and hypoxanthine.

Following triggering and Cre-mediated genomic rearrangement, the selection elements remain unchanged, allowing continued maintenance of the three post-triggering integrated constructs using a single antibiotic in medium lacking hypoxanthine and thymidine.

Viral proteins needed for AAV virion formation are inhibited by host cell mechanisms. Inhibition of these host cell mechanisms to maximize AAV viral titers in the stable cell lines described herein include, but are not limited to: knocking out PKR (PKR KO) (pathway is responsible for inhibition of viral proteins) in the starting cell line (P0), introducing a mutant EIF2alpha (in the PKR pathway) in the starting cell line (P0), and/or manipulating or modulating virus-associated (VA) RNAs (VA RNAs, an inhibitor of PKR). Virus-associated (VA) RNAs from adenovirus act as small-interference RNAs and are transcribed from the vector genome. These VA RNAs can trigger the innate immune response. Moreover, VA RNAs are processed to functional viral miRNAs and disturb the expression of numerous cellular genes. Therefore, VA-deleted adenoviral vector production constructs (AdVs) lacking VA RNA genes, or having modified VA RNA, would be advantageous. However, VA-deleted AdVs do not produce commercially sufficient quantities of AAV titers (e.g. resulting in fewer and poor-quality virions). Conversely, overexpressing VA RNA also results in a low titer of AAV production that would not be commercially feasible for scale-up. Thus, developing conditional VA RNA constructs, and combining any of those optimized constructs with the conditional helper constructs described herein, will provide commercially relevant, high-quality virions from the AAV production systems as described herein. All three of these strategies can be done in any combination.

VA RNA is also an inhibitor of PKR, which is involved in a pathway responsible for inhibiting AAV viral protein synthesis. In particular, PKR phosphorylates EIF2alpha, which results in inhibition of viral protein synthesis. FIG. 4A shows a schematic of VA RNA inhibition of PKR and the PKR pathway is shown below at left. The structure of VA RNA, which is a double stranded RNA (dsRNA) is shown in FIG. 4B.

While the limited interactions between VA RNA, PKR, and EIF2alpha are understood, PKR is a major kinase that may self-phosphorylate and EIF2alpha may be phosphorylated by other kinases. As such, three strategies (PKR KO, EIF2alpha mutation, manipulation of VA RNA) are being developed for use in any combination in the AAV production systems described herein.

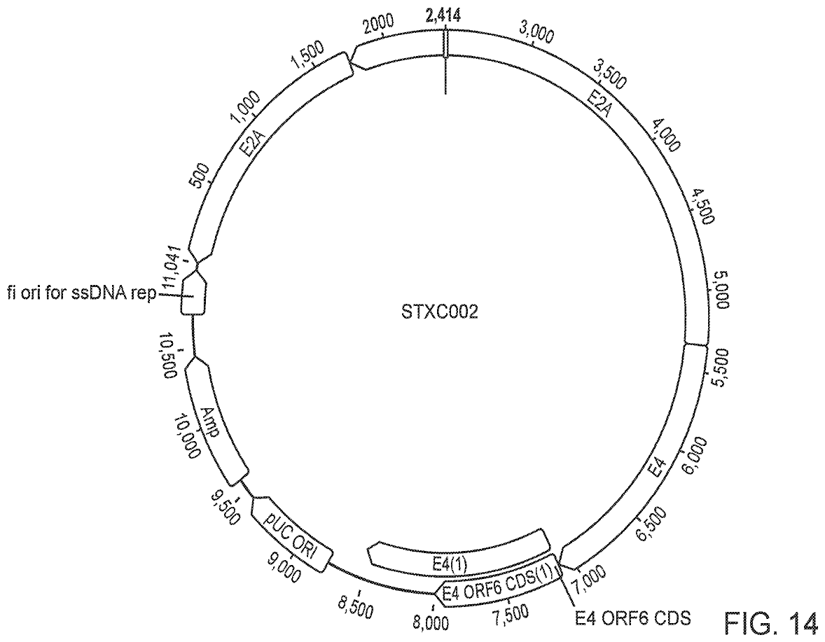

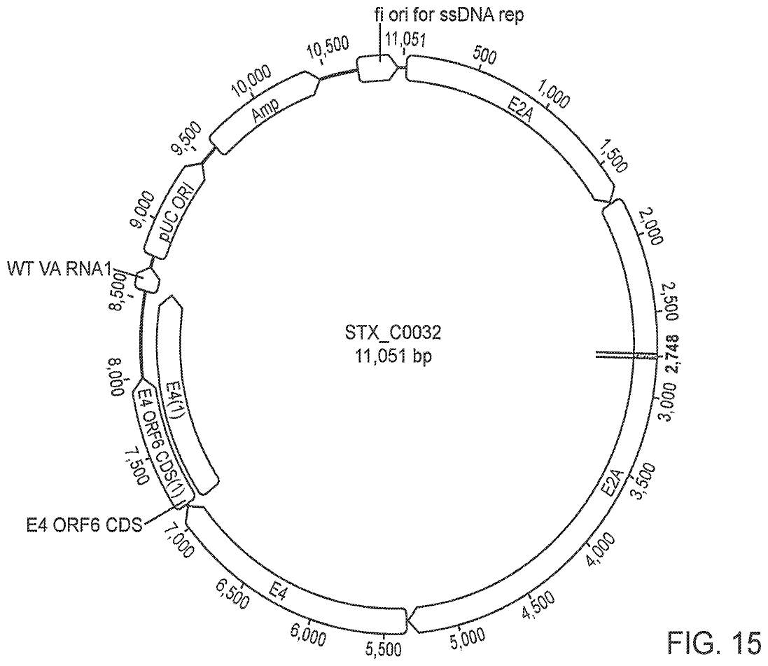

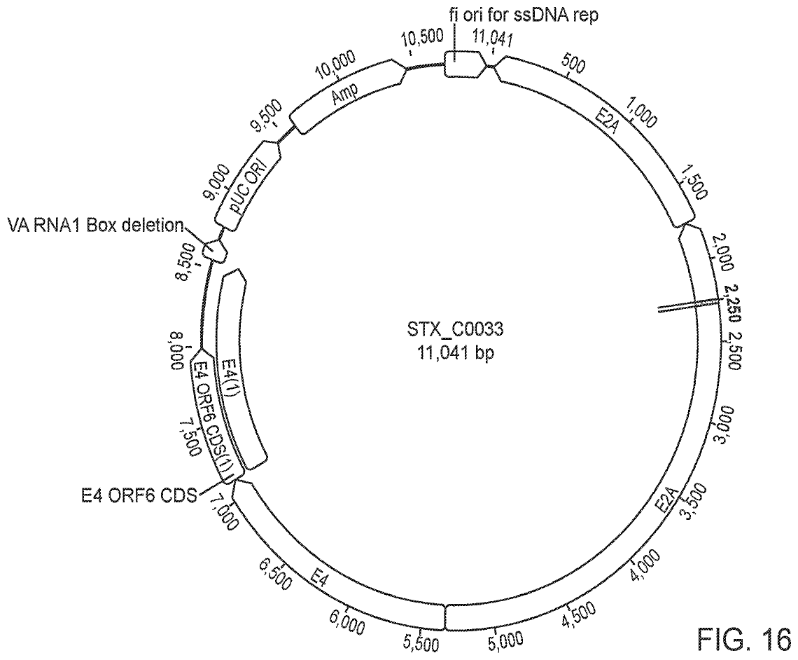

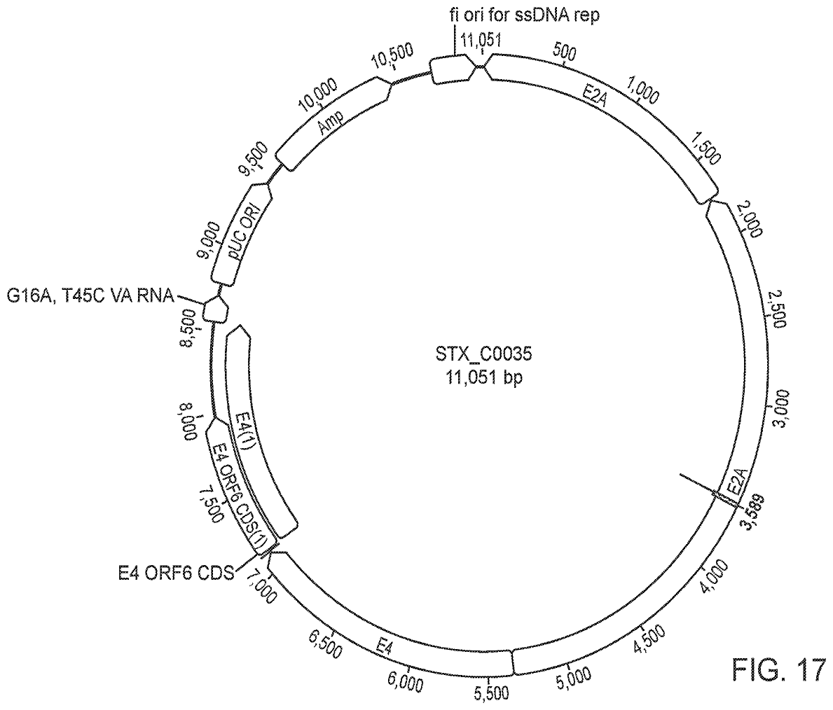

Thus, an option for overcoming the general antiviral effects of mammalian cell production of AAV virions is to modify expression of VA RNA. Therefore, VA-deleted adenoviral vector production constructs (AdVs) lacking VA RNA genes, or having modified VA RNA, have been designed and are described herein in FIG. 2C and FIGS. 4-15. It is noted that VA-deleted AdVs do not produce commercially sufficient quantities of AAV titers (e.g. resulting in fewer and poor-quality virions). Conversely, overexpressing VA RNA also results in a low titer of AAV virion production that would not be commercially feasible for scale-up. Thus, developing conditional VA RNA constructs, and combining any of those optimized constructs with the conditional helper constructs described herein, will provide commercially relevant, high-quality virions from the AAV production systems as described herein. FIG. 2C and FIGS. 4-15 illustrate various modified and inducible mutant VA RNA constructs and their effects on virion production. These various approaches provide numerous benefits over current systems for AAV production.

### 6.3. Conditional Expression

In a first aspect, the stable cell lines are provided. In some embodiments, the stable cell lines are mammalian stable cell lines. The cells are capable of conditionally producing recombinant AAV (rAAV) virions. In some embodiments, the cells are capable of conditionally producing rAAV virions. In some embodiments, said rAAV virions package an expressible payload. In some embodiments, said rAAV virions package a sequence encoding a payload. In preferred embodiments, production of virions is not conditioned on the presence of an episome or independent plasmid within the cell.

In some embodiments, expression of AAV Rep is conditional. In some embodiments, expression of AAV Rep and Cap proteins is conditional. In certain embodiments, expression of AAV Rep and Cap proteins is conditioned on addition of at least a first expression triggering agent to the cell culture medium. In certain embodiments, expression of AAV Rep and Cap proteins is conditioned on addition of a first expression triggering agent and a second expression triggering agent to the cell culture medium.

In a system with a triggering agent, doxycycline is a suitable agent. In certain embodiments, a Tet inducible promoter can be utilized that is under the control of doxycycline. Alternatively, a cumate inducible promoter system can be utilized in which the cumate inducible promoter is under the control of cumate.

In a system with a triggering agent, doxycycline is a suitable agent. In certain embodiments, doxycycline is used to the control a Tet inducible promoter. Alternatively, a cumate inducible promoter system can be utilized instead of a Tet inducible promoter, which is under the control of cumate.

Any suitable inducible excising agent (e.g., recombinase) can be utilized. An excising agent can be a recombinase. An excising agent can be a site-specific recombinase. An excising agent can target a recombination site. Examples of suitable inducible excising agents include Cre and a flippase. The Cre element can be hormone activated Cre, or light inducible Cre. A recombination site can be a lox site. A lox site can be a loxP site. A recombination site can be an FRT site.

The Flippase recombinase system is based on Flp-FRT recombination, a site-directed recombination technology used to manipulate DNA under controlled conditions in vivo. It is analogous to Cre-lox recombination but involves the recombination of sequences between short flippase recognition target (FRT) sites by the recombinase flippase (Flp) derived from the 2 μ plasmid of baker's yeast Saccharomyces cerevisiae. The Flp protein, much like Cre, is a tyrosine family site-specific recombinase.

In typical embodiments, the cells do not express cytotoxic levels of Rep protein prior to addition of both the first expression and second triggering agents to the cell culture medium. In certain embodiments, the cells do not express cytostatic levels of Rep protein prior to addition of both the first and second expression triggering agents to the cell culture medium. In certain embodiments, the average concentration of Rep protein within the cells is less than the amount prior to addition of both of the first and second expression triggering agents to the cell culture medium. In some embodiments, expression of Rep and Cap proteins becomes constitutive after addition of all of the at least first expression triggering agents to the cell culture medium.

In some embodiments, expression of at least one adenoviral helper protein is conditional.

In certain embodiments, expression of the at least one adenoviral helper protein is conditioned on addition of at least a third expression triggering agent to the cell culture medium. In particular embodiments, the third expression triggering agent is the same as the first expression triggering agent. In certain embodiments, expression of adenoviral helper proteins is conditioned on addition of a third expression triggering agent and a fourth expression triggering agent to the cell culture medium. In particular embodiments, the fourth expression triggering agent is the same as the second expression triggering agent. In particular embodiments, the third expression triggering agent is the same as the first expression triggering agent and the fourth expression triggering agent is the same as the second expression triggering agent.

In some embodiments, continued expression of adenoviral helper proteins following triggering of expression by contact of the cell with the at least third expression triggering agent requires the presence of only the third expression triggering agent in the cell culture medium. In certain embodiments, the third triggering agent is the same as the first triggering agent.

In some embodiments, expression of at least one adenoviral helper RNA is conditional. In certain embodiments, the adenoviral helper proteins comprise Ad E2A. In certain embodiments, the adenoviral helper proteins comprise Ad E4. In some embodiments, the adenoviral helper protein is tagged. A tag can be a protein tag. A protein tag can be a FLAG tag. In some embodiments, E2A is FLAG-tagged. In some embodiments, E4 is FLAG-tagged.

In particular embodiments, the adenoviral helper RNA is a VA RNA. In particular embodiments, the adenoviral helper RNA is an inducible VA RNA construct. In some embodiments, the VA RNA is a mutant VA RNA. In some embodiments, the VA RNA is a transcriptionally dead VA RNA. In some embodiments, the VA RNA is under the control of a U6 promoter.

In some embodiments, the third expression triggering agent is a tetracycline. In certain embodiments, the tetracycline is doxycycline (“Dox”). In some embodiments, the fourth expression triggering agent is an estrogen receptor ligand. In certain embodiments, the estrogen receptor ligand is a selective estrogen receptor modulator (SERM). In particular embodiments, the estrogen receptor ligand is tamoxifen.

In some embodiments of the stable cell line, expression of the payload is conditioned on addition of at least a fifth expression triggering agent to the cell culture medium. In some embodiments, expression of the payload is not conditioned on addition of an expression triggering agent to the cell culture medium.

In some embodiments, expression of Rep and Cap proteins, adenoviral helper proteins, and the payload becomes constitutive after addition of only one expression triggering agent to the cell culture medium. In certain embodiments, expression of Rep and Cap proteins and the adenoviral helper proteins becomes constitutive after addition of only one expression triggering agent to the cell culture medium.

In certain embodiments, the one expression triggering agent is the first expression triggering agent. In certain embodiments, the first expression triggering agent is a tetracycline. In particular embodiments, the first expression triggering agent is doxycycline.

### 6.4. Synthetic Nucleic Acid Constructs

In typical embodiments, the nuclear genome of the cell of the stable cell line comprises a plurality of integrated synthetic nucleic acid constructs. Typically, each of the plurality of synthetic nucleic acid constructs is separately integrated into the nuclear genome of the cell. In some embodiments, only a single non-auxotrophic selection is required to maintain all of the plurality of synthetic nucleic acid constructs stably within the nuclear genome of the cells. In some embodiments, antibiotic resistance is required to maintain the plurality of synthetic constructs stably within the nuclear genomes of the cells. In some embodiments, both a non-auxotrophic selection and antibiotic resistance is required to maintain the plurality of synthetic constructs stably within the nuclear genomes of the cells. I

In some embodiments, the nuclear genome of the cell comprises two integrated synthetic constructs.

In some embodiments, the nuclear genome of the cell comprises three integrated synthetic constructs. In particular embodiments, the first integrated synthetic construct comprises conditionally expressible AAV Rep and Cap coding sequences; the second integrated synthetic construct comprises a conditionally expressible Cre coding sequence and conditionally expressible adenoviral helper protein coding sequences; and the third integrated synthetic construct comprises expressible coding sequences for the payload.

### 6.4.1. Construct 1 (AAV Rep/Cap Construct)

Disclosed herein are polynucleotide constructs encoding for a Rep and Cap polypeptide. Provided herein is a first polynucleotide construct, which encodes for Rep and Cap and comprises spacer or excisable elements. This first polynucleotide construct is also referred to as a Rep/Cap construct, and/or “AAV Rep/Cap Construct.”

These polynucleotide constructs are designed to be stably integrated into a cell line and only be triggered to produce AAV Rep and Cap polypeptides in the presence of an excising element. In some embodiments, the first integrated synthetic construct comprises conditionally expressible AAV Rep and Cap coding sequences.

The Rep sequence can encode Rep from any desired AAV serotype. In some embodiments, the encoded Rep protein is drawn from the same serotype as the Cap protein. In some embodiments, the encoded Rep protein is drawn from a different serotype from the Cap protein. In particular embodiments, the encoded Rep protein includes, but is not limited to, a Rep protein from AAV serotypes AAV-1, AAV-2, AAV-3, AAV-4, AAV-5, AAV-6, AAV-7, AAV-8, AAV-9, AAV-10 and AAV-11, or chimeric combinations thereof.

The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol, 45: 555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively (see also U.S. Pat. Nos. 7,282,199 and 7,790,449 relating to AAV-8); the AAV-9 genome is provided in Gao et al. Virol, 78: 6381-6388 (2004); the AAV-10 genome is provided in Mol Ther, 13(1): 67-76 (2006); and the AAV-11 genome is provided in Virology, 330(2): 375-383 (2004).

In the exemplary embodiments illustrated in FIG. 3A, prior to the cell being contacted with the first expression triggering agent, the Rep coding sequence is interrupted by an intervening spacer.

In certain embodiments, the intervening spacer segment comprises, from 5′ to 3′, a first spacer segment, a second spacer segment, and a third spacer segment.

In particular embodiments, the first spacer segment comprises a 5′ splice site (5′SS) 5′ to the first spacer element. In some embodiments, the first spacer segment comprises a nucleic acid sequence having at least 80% identity to SEQ ID NO: 1.

In some embodiments, the second spacer segment comprises a polynucleotide encoding a detectable protein marker flanked by lox sites. In certain embodiments, the detectable protein marker is a fluorescent protein. In particular embodiments, the fluorescent protein is a green fluorescent protein (GFP). In specific embodiments, the GFP is EGFP. In particular embodiments, the fluorescent protein is a blue fluorescent protein (BFP). Screening for the fluorescent marker can be used to confirm integration of the construct into the cell genome, and can subsequently be used to confirm excision of the intervening spacer segment. In some embodiments, the second spacer segment further comprises a polyA sequence. In certain embodiments, the poly A sequence comprises a rabbit beta globin (RBG) polyA. In some embodiments, the second spacer segment further comprises a first 3′ splice site (3′SS) between the first lox site and the polynucleotide encoding the protein marker.

In some embodiments, the second spacer segment comprises a nucleic acid sequence having at least 80% identity to SEQ ID NO: 2.

In some embodiments, the third spacer segment further comprises a second 3′ splice site (3′SS). In particular embodiments, the second 3′ splice site is positioned 3′ to the second lox site.

In some embodiments, the third spacer segment comprises a nucleic acid sequence having at least 80% identity to SEQ ID NO: 3.

In various embodiments, the Rep coding sequence is operatively linked to an endogenous P5 promoter. In various embodiments, the Rep coding sequence is operatively linked to an endogenous P19 promoter. In some embodiments, the intervening spacer is inserted into the Rep coding sequence at a position downstream of the P19 promoter.

In some embodiments, the Rep coding sequence is 5′ to the Cap coding sequence. In certain embodiments, the Cap coding sequence is operatively linked to an endogenous P40 promoter.

In various embodiments, the Cap protein is selected from the capsid of an avian AAV, bovine AAV, canine AAV, equine AAV, primate AAV, non-primate AAV, and ovine AAV, and modifications, derivatives, or pseudotypes thereof.

In some embodiments, the capsid is a capsid selected from AAV1, AAV2, AAV3, AAV4, AAV5, AAV6, AAV7, AAV8, AAV9, AAV 10, AAV11, AAV 12, AAV13, AAV 14, AAV 15 and AAV 16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16 or AAVhu68 (described in WO2020/033842, incorporated herein by reference in its entirety). The hu68 capsid is described in WO 2018/160582, incorporated herein by reference in its entirety.

In some embodiments, the capsid is a derivative, modification, or pseudotype of AAV1, AAV2, AAV3, AAV4, AAV5, AAV6, AAV7, AAV8, AAV9, AAV 10, AAV11, AAV 12, AAV 13, AAV 14, AAV 15 and AAV 16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16 or AAVhu68.

In some embodiments, capsid protein is a chimera of capsid proteins from two or more serotype selected from AAV1, AAV2, rAAV3, AAV4, AAV5, AAV6, AAV7, AAV8, AAV9, AAV 10, AAV11, AAV 12, AAV13, AAV 14, AAV 15 and AAV 16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16 (described in WO2020/033842, incorporated herein by reference in its entirety). In certain embodiments, the capsid is an rh32.33 capsid, described in U.S. Pat. No. 8,999,678, incorporated herein by reference in its entirety.

In particular embodiments, the capsid is an AAV1 capsid. In particular embodiments, the capsid is an AAV5 capsid. In particular embodiments, the capsid is an AAV9 capsid.

In various embodiments, the first integrated construct further comprises a first mammalian cell selection element.

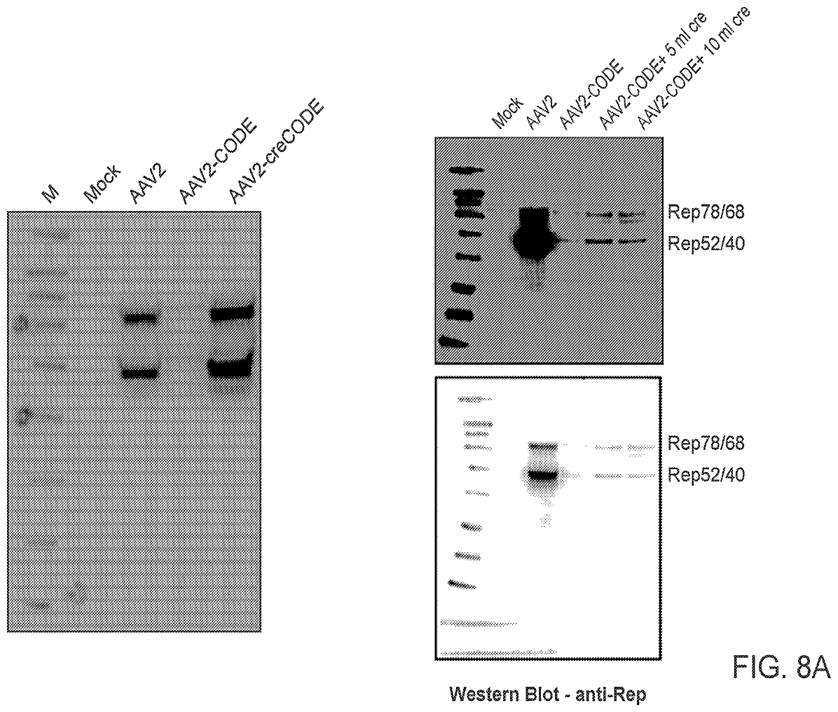

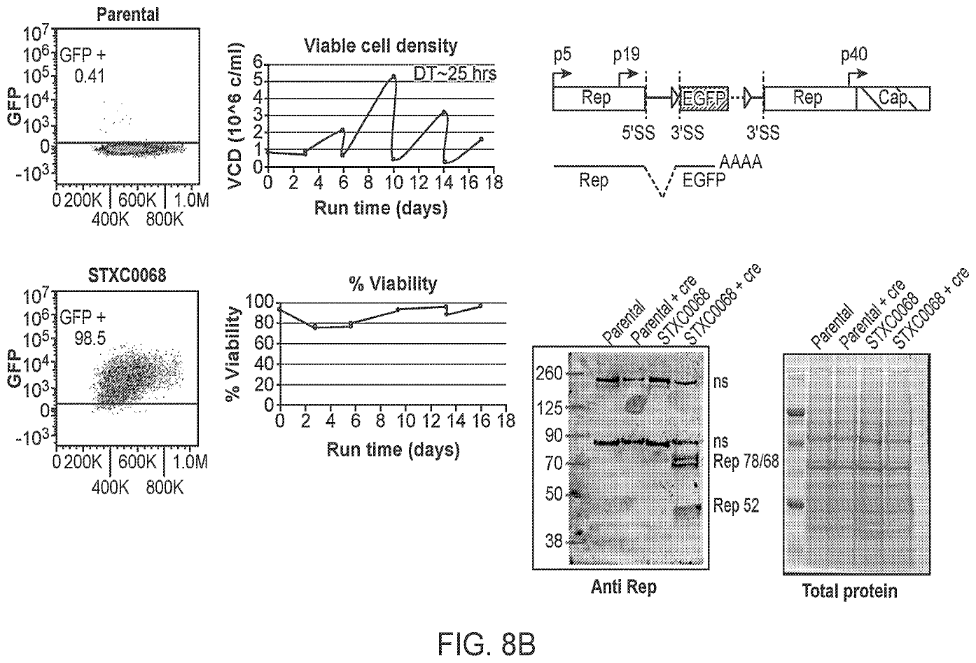

In some embodiments, the inducible Rep and Cap construct is as shown in FIG. 8B. In some embodiments, the inducible polynucleotide construct encoding for Rep and Cap encodes for a first part of a Rep polypeptide, a second part of a Rep polypeptide, a Cap polypeptide, and an excisable element. The excisable element may be positioned between the first part of the Rep polypeptide and the second part of the Rep polypeptide. The excisable element may, thus, interrupt the sequence encoding for the Rep polypeptide at any point along the sequence encoding for Rep. Without excision of the excisable element, Rep is minimally expressed or not expressed at all. In some embodiments, the Rep polypeptide is a wildtype Rep polypeptide. In other embodiments, the Rep polypeptide is a mutant Rep polypeptide. In some embodiments, the Cap polypeptide is a wildtype Cap polypeptide. In other embodiments, the Cap polypeptide is a mutant Cap polypeptide. In some embodiments, the excisable element comprises an intron, an exon, or an intron and an exon. In particular embodiments, the excisable element from 5′ to 3′ comprises a 5′ splice site; a first spacer segment comprising a first intron; a second spacer segment comprising: a first lox sequence, a 3′ splice site, an exon, a stop signaling sequence, a second lox sequence; and a third spacer segment comprising a second intron. The first spacer segment and the third spacer segment may be excised by endogenous cellular machinery.

In some embodiments, the second spacer segment in the excisable element is excised by Cre. Cre may be provided as any form of exogenous Cre, such as Cre gesicles. Cre may also be encoded for by a second polynucleotide construct. In some embodiments, a construct encoding for adenoviral helper proteins also encodes for Cre. In some embodiments, the second polynucleotide construct is also inducible, for example, as described below in Section 4.3.2.

In some embodiments, expression of the Rep and Cap are driven by native promoters, including P5, P19, P40, or any combination thereof. In some embodiments the exon of the excisable element may be any detectable marker. For example, detectable markers contemplated herein include luminescent markers, fluorescent markers, or radiolabels. Fluorescent markers include, but are not limited to, EGFP, GFP, BFP, RFP, or any combination thereof.

In some embodiments, the Rep/Cap construct is a polynucleotide construct comprising: a) a sequence of a first part of a Rep gene; b) sequence of a second part of the Rep gene; c) a sequence of a Cap gene; and d) an excisable element positioned between the first part of the sequence of Rep gene and the second part of the sequence of the Rep gene. In some embodiments, the excisable element comprises a stop signaling sequence. In some embodiments, the excisable element comprises a rabbit beta globin intron. In some embodiments, the excisable element comprises an exon. In some embodiments, the excisable element comprises an intron and an exon. In some embodiments, the excisable element comprises an intron. In some embodiments, two splice sites are positioned between the sequence of the first part of the Rep gene and the sequence of the second part of the Rep gene. In some embodiments, the two splice sites are a 5′ splice site and a 3′ splice site. In some embodiments, the 5′ splice site is a rabbit beta globin 5′ splice site. In some embodiments, the 3′ splice site is a rabbit beta globin 3′ splice site. In some embodiments, three splice sites are positioned between the sequence of the first part of the Rep gene and the sequence of the second part of the Rep gene. In some embodiments, the three splice sites are a 5′ splice site, a first 3′ splice site, and a second 3′ splice site. In some embodiments, a first 3′ splice site is a duplicate of the second 3′ splice site. In some embodiments, the first 3′ splice site is a rabbit beta globin 3′ splice site. In some embodiments, the second 3′ splice site is a rabbit beta globin 3′ splice site. In some embodiments, the excisable element comprises a recombination site. In some embodiments, the recombination site is a lox site or FRT site. In some embodiments, the lox site is a loxP site. In some embodiments, the excisable element comprises from 5′ to 3′: a) the 5′ splice site; b) a first recombination site; c) the first 3′ splice site; d) a stop signaling sequence; e) a second recombination site; and f) the second 3′ splice site. In some embodiments, the excisable element comprises from 5′ to 3′: a) the 5′ splice site; b) a first spacer segment; c) a second spacer segment comprising: i) a first recombination site; ii) the first 3′ splice site; iv) a stop signaling sequence; and v) a second recombination site; and d) a third spacer segment comprising the second 3′ splice site. In some embodiments, the first spacer sequence comprises an intron. In some embodiments, the first spacer segment comprises a sequence having at least 70%, 80%, 90%, 95%, 99%, or 100% sequence identity to SEQ ID NO: 1. In some embodiments, the second spacer segment comprises a sequence having at least 70%, 80%, 90%, 95%, 99%, or 100% sequence identity to SEQ ID NO: 2. In some embodiments, the third spacer segment comprises a sequence having at least 70%, 80%, 90%, 95%, 99%, or 100% sequence identity to SEQ ID NO: 3. In some embodiments, the third spacer segment comprises an intron. In some embodiments, the first spacer segment and the third spacer segment are capable of being excised by endogenous cellular machinery. In some embodiments, the second spacer segment comprises an exon. In some embodiments, the second spacer segment further comprises a polyA sequence. In some embodiments, the polyA sequence is 3′ of the exon. In some embodiments, the polyA sequence comprises a rabbit beta globin (RBG) polyA sequence. The polynucleotide construct of any one of claims, wherein the second spacer segment comprises from 5′ to 3′: a) a first recombination site; b) the first 3′ splice site; c) an exon; d) a stop signaling sequence; and e) a second recombination site. In some embodiments, the first recombination site is a first lox sequence and the second recombination site is a second lox sequence. In some embodiments, the first lox sequence is a first loxP sequence and a second lox sequence is a second loxP sequence. In some embodiments, the first recombination site is a first FRT site and the second recombination site is a second FRT site. In some embodiments, the stop signaling sequence is a termination codon of the exon or a polyA sequence. In some embodiments, the polyA sequence comprises a rabbit beta globin (RBG) polyA sequence. In some embodiments, the exon encodes a detectable marker or a selectable marker. In some embodiments, the detectable marker comprises a luminescent marker or a fluorescent marker. In some embodiments, the fluorescent marker is GFP, EGFP, RFP, CFP, BFP, YFP, or mCherry. In some embodiments, the second spacer segment is excisable by a recombinase. In some embodiments, the recombinase is a site-specific recombinase. The polynucleotide construct of any one of claims, wherein the recombinase is a Cre polypeptide or a Flippase polypeptide. The polynucleotide construct of any one claims X, wherein the Cre polypeptide is fused to a ligand binding domain. In some embodiments, the ligand binding domain is a hormone receptor. In some embodiments, the hormone receptor is an estrogen receptor. In some embodiments, the estrogen receptor comprises a point mutation. In some embodiments, the estrogen receptor is ERT2. The polynucleotide construct of any one claims X, wherein the recombinase is a Cre-ERT2 polypeptide. The polynucleotide construct of claim 9, wherein the recombinase is encoded by a second polynucleotide construct or exogenously provided. In some embodiments, the Rep gene codes for Rep polypeptides. In some embodiments, the Cap gene codes for Cap polypeptides. In some embodiments, transcription of the Rep gene and the Cap gene are driven by native promoters. In some embodiments, the native promoters comprise P5, P19, and P40. In some embodiments, the Rep polypeptides are wildtype Rep polypeptides. In some embodiments, the Rep polypeptides comprise Rep78, Rep68, Rep52, and Rep40. In some embodiments, a truncated replication associated protein comprising a polypeptide expressed from the sequence of first part of a Rep gene and the exon is capable of being expressed in the absence of the recombinase. In some embodiments, the Cap polypeptides are wildtype Cap polypeptides. In some embodiments, the Cap polypeptides are AAV capsid proteins. In some embodiments, the AAV capsid proteins comprise VP1, VP2, and VP3. In some embodiments, a serotype of the AAV capsid proteins is selected from the group consisting of AAV1, AAV2, AAV3, AAV4, AAV5, AAV6, AAV7, AAV8, AAV9, AAV 10, AAV11, AAV 12, AAV13, AAV 14, AAV 15 and AAV 16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, and AAVhu68.

In some embodiments, the Rep/Cap construct further comprises a sequence coding for a selectable marker. In some embodiments, the selectable marker is a mammalian cell selection element. In some embodiments, the selectable marker is an auxotrophic selection element. In some embodiments, the auxotrophic selection element codes for an active protein. In some embodiments, the active protein is DHFR. In some embodiments, the auxotrophic selection coding sequence encodes an inactive protein that requires expression of a second auxotrophic selection coding sequence for activity. In some embodiments, the second auxotrophic selection coding sequence encodes for DHFR Z-Cter or DHFR Z-Nter. In some embodiments, the inactive protein comprises a DHFR Z-Nter or DHFR Z-Cter In some embodiments, the selectable marker is DHFR Z-Nter or DHFR Z-Cter. The polynucleotide construct of any one of claims 2-6, wherein the DHFR Z-Nter comprises a sequence having at least 70%, 80%, 90%, 95%, 99%, or 100% sequence identity to SEQ ID NO: 4. The polynucleotide construct of any one of claims 2-6, wherein the DHFR Z-Cter comprises a sequence having at least 70%, 80%, 90%, 95%, 99%, or 100% sequence identity to SEQ ID NO: 5. In some embodiments, the selectable marker is an antibiotic resistance protein. In some embodiments, the selectable marker is a split intein linked to an N-terminus of the antibiotic resistance protein or split intein linked to a C-terminus of the antibiotic resistance protein. In some embodiments, the selectable marker is a leucine zipper linked to an N-terminus of the antibiotic resistance protein or leucine zipper linked to a C-terminus of the antibiotic resistance protein. the antibiotic resistance protein is for puromycin resistance or blasticidin resistance.

In some embodiments, the Rep/Cap construct is in a vector. In some embodiments, the Rep/Cap construct is in a plasmid. In some embodiments, the Rep/Cap construct is in a bacterial artificial chromosome or yeast artificial chromosome. In some embodiments, the Rep/Cap construct is a synthetic nucleic acid construct. In some embodiments, the Rep/Cap construct comprises a sequence having at least 70%, 80%, 90%, 95%, 99%, or 100% sequence identity to any one of SEQ ID NO: 1-SEQ ID NO: 3, SEQ ID 6-SEQ ID NO: 8, or SEQ ID NO: 32. In some embodiments, the Rep/Cap construct has at least 70%, 80%, 90%, 95%, 99%, or 100% sequence identity to any one of SEQ ID NO: 1-SEQ ID NO: 3, SEQ ID 6-SEQ ID NO: 8, or SEQ ID NO: 32.

In some embodiments, the Rep/Cap construct further comprises a sequence coding for VA RNA. In some embodiments, the sequence coding for VA RNA is a transcriptionally dead sequence. In some embodiments, the sequence coding for VA RNA comprises at least two mutations in the internal promoter. In some embodiments, expression of VA RNA is driven by a U6 promoter. The polynucleotide construct of any one of claims X, comprising upstream of the sequence coding for VA RNA gene sequence, from 5′ to 3′: a) a first part of a U6 promoter sequence; b) a first recombination site; c) a stuffer sequence; d) a second recombination site; e) a second part of a U6 promoter sequence. In some embodiments, the stuffer sequence is excisable by the recombinase. In some embodiments, the stuffer sequence comprises a sequence encoding a gene. In some embodiments, the stuffer sequence comprises a promoter. In some embodiments, the promoter is a constitutive promoter. In some embodiments, the promoter is a CMV promoter.

A major advantage of the inducible polynucleotide constructs disclosed herein encoding for Rep and Cap include that upon stable integration into a mammalian cell line, expression of Rep and Cap is inducible even in the absence of a transfection agent or a plasmid. In some embodiments, the stable cell line populations disclosed herein are homogeneous. For example, at least 70%, at least 80%, at least 90%, at least 95%, or 100% of the stable cell population comprises the stably integrated polynucleotide construct encoding for Rep and Cap proteins.

### 6.4.2. Construct 2 (Adenoviral Helper Construct (Provides E2A/E4))

Provided herein is a second polynucleotide construct, which encodes for one or more adenoviral helper proteins. This second polynucleotide construct is also referred to as an inducible helper construct (e.g., Adenoviral Helper Construct (provides E2A/E4)). In certain embodiments, the adenoviral helper construct provides inducible production of E2A/E4. In some embodiments, an adenoviral helper protein further comprises a protein tag. A protein tag can be a FLAG tag. In some embodiments, E2A is a FLAG tagged E2A. In some embodiments, E4 is a FLAG tagged E4. A protein tag, such as a FLAG tag, can be used to screen for or to confirm integration the second polynucleotide construct and expression of the adenoviral helper protein from the second polynucleotide construct in a cell after induction.

In some embodiments, the second integrated synthetic construct comprises conditionally expressible Cre recombinase and conditionally expressible adenovirus helper proteins. In the exemplary embodiments illustrated in FIG. 2A, prior to the cell being contacted with at least a third expression triggering agent, the second integrated construct comprises, from 5′ to 3′: an inducible promoter, a Cre coding sequence, a first polyA sequence, adenoviral helper protein coding sequences, a second polyA sequence, a constitutive promoter, a coding sequence for a protein that is responsive to the first expression triggering agent, and a second mammalian cell selection element.

In typical embodiments, the Cre coding sequence is operatively linked to the inducible promoter. In various embodiments, the inducible promoter comprises an element responsive to the third expression triggering agent. In certain embodiments, the inducible promoter comprises a plurality of tetracycline (Tet) operator elements capable of binding to a Tet responsive activator protein in the presence of a tetracycline. In some embodiments, the plurality of tetracycline (Tet) operator elements form a Tetracycline Responsive element (TRE). In some embodiments, the TRE comprises seven repeats of a 19 base pair operator sequence. In further embodiments, the TRE comprises seven repeats of a 19 base pair operator sequence upstream of a minimal CMV promoter sequence.

In some embodiments, the second construct further comprises an element responsive to a fourth expression triggering agent. In certain embodiments, the fourth expression triggering agent-responsive element comprises a plurality of hormone-response elements. In particular embodiments, the hormone-response elements are estrogen responsive elements (EREs). In various embodiments, the third expression triggering element is the same as the first expression triggering element, and the fourth expression triggering element is the same as the second expression triggering element.

In some embodiments, the Cre coding sequence is flanked by a first lox site and a second lox site.

In some embodiments, the inducible promoter comprises a plurality of Tet operator elements capable of binding to a Tet responsive activator protein in the presence of a third expression triggering agent. In particular embodiments, the third expression triggering agent is the same as the first expression triggering agent.

In some embodiments, the first polyA sequence is positioned between the Cre coding sequence and adenoviral helper protein coding sequences that encode one or both of adenovirus E2A and E4. The strong 3′ polyadenylation signal positioned upstream (5′ to) the coding sequences for the adenovirus helper proteins prevents basal expression of the downstream adenoviral helper genes, E2A and E4.

In some embodiments, the further segment shown in FIG. 2C provides for inducible production of VA-RNA from construct 2.

In this embodiment, the further segment includes a Cre-inducible U6 promoter. The U6 promoter is split into 2 parts separated by a Lox flanked stuffer sequence. The U6 promoter is inactive because of the presence of the stuffer sequence. Cre mediated excision of the stuffer activates the U6 promoter. The U6 promoter drives the expression of transcriptionally dead mutants of VA RNA1 (a preferred embodiment is a double point mutant G16A-G60A). Other embodiments provide for alternative sources of VA-RNA.

In various embodiments, the coding sequence for the first expression triggering agent-responsive protein is operatively linked to a CMV promoter. In some embodiments, the coding sequence for the first expression triggering agent-responsive protein comprises a coding sequence for the Tet responsive activator protein. In particular embodiments, the Tet responsive activator protein is Tet-on-3G activator protein.

In various embodiments, the second mammalian cell selection element confers antibiotic resistance. In particular embodiments, the antibiotic resistance conferring element is a blasticidin resistance gene.

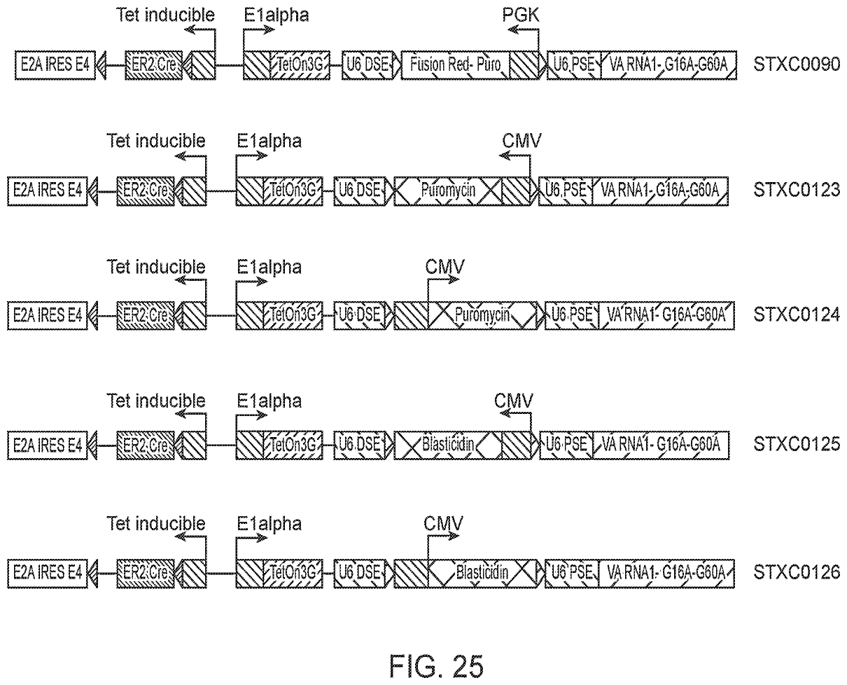

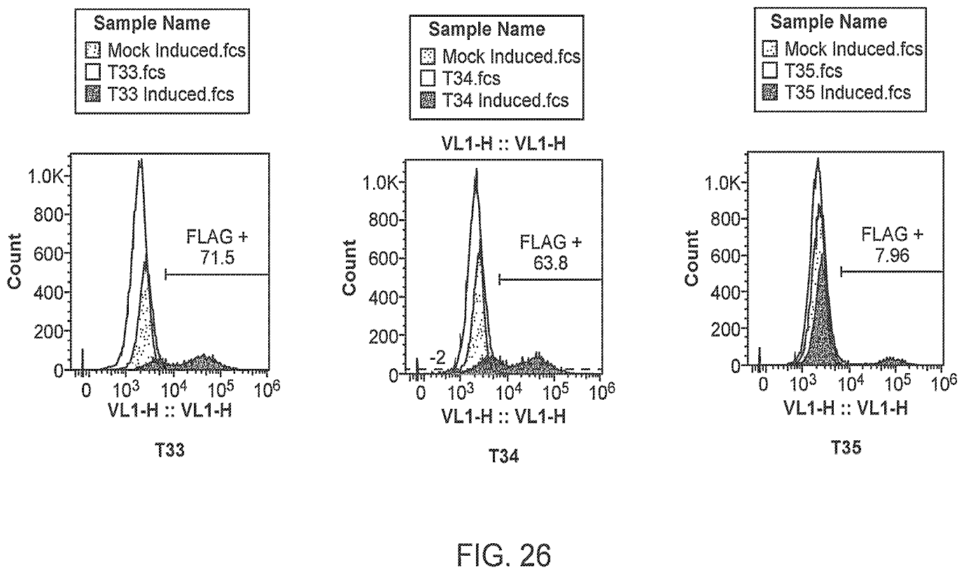

In some embodiments, the inducible helper polynucleotide construct is as shown at left or at right in FIG. 25. Multiple inducible helper polynucleotide constructs are contemplated herein. In some embodiments, said inducible helper polynucleotide constructs encode for one or more adenoviral helper proteins, such as VA RNA, E2A, E4, or any combination thereof. In some embodiments, the present disclosure provides for an inducible polynucleotide construct encoding for a mutated VA RNA gene sequence. In some embodiments, the mutations to VA RNA render its internal promoters inactive. For example, as shown in FIG. 25 (at left), the inducible helper polynucleotide construct may comprise from 5′ to 3′ a first part of a U6 promoter sequence, a first lox sequence, a stuffer sequence, a second lox sequence, and a second part of a U6 promoter sequence. The stuffer sequence may be any polynucleotide sequence and is excised by Cre. Cre may be exogenously provided, such as in the form of Cre gesicles. Cre may also be encoded for in the same inducible helper polynucleotide construct and expression of Cre may be conditioned on the presence of at least two triggering agents, such as doxycycline and tamoxifen. Cre may be a hormone activated Cre.

In other embodiments, instead of a mutated VA RNA gene sequence, the inducible helper constructs may comprise a constitutively expressed VA RNA that is not mutated, for example, as shown in FIG. 25 (at right).

In some embodiments, the inducible helper polynucleotide construct also encodes for one or more helper proteins, a self-excising element upstream of the one or more helper proteins, and an inducible promoter upstream of the self-excising element. Expression of the self-excising element may be driven by a Tet-On-3G system. For example, the construct may comprise a Tet-On 3G gene sequence, wherein expression is driven by an Elalpha promoter. The Elalpha promoter may be a mutated Elalpha promoter. The mutated Elalpha promoter can have a sequence of:
ggatctgcgatcgctccggtgcccgtcagtgggcagagcgcacatcgcccacagtccccgagaagttggggggaggggtcggcaa ttgaacgggtgcctagagaaggtggcgcggggtaaactgggaaagtgatgtcgtgtactggctccgcctttttcccgagggtggggg agaaccgtatgtaagtgcagtagtcgccgtgaacgttctttttcgcaacgggtttgccgccagaacacagctgaagcttcgaggggctc gcatctctccttcacgcgcccgccgccctacctgaggccgccatccacgccggttgagtcgcgttctgccgcctcccgcctgtggtgc ctcctgaactgcgtccgccgtctaggtaagtttaaagctcaggtcgagaccgggcctttgtccggcgctcccttggagcctacctagac tcagccggctctccacgctttgcctgaccctgcttgctcaactctacgtctttgtttcgttttctgttctgcgccgttacagatccaagctgtg accggcgcctac (SEQ ID NO: 20).

In the presence of a first triggering agent, such as doxycycline, Tet-On-3G is able to bind the Tet inducible promoter. Upon this binding event, the Tet inducible promoter drives expression of the self-excising element. In some embodiments, the self-excising element is a hormone activated Cre. In the presence of a second triggering agent, such as tamoxifen, and upon expression of Cre, Cre self-excises itself leading to expression of downstream adenoviral helper proteins. Thus, mammalian cell lines stably integrated with the inducible helper constructs disclosed herein only express adenoviral helper proteins in the presence of at least two triggering agents (e.g., doxycycline and tamoxifen).

In some embodiments, an inducible helper construct is a polynucleotide construct coding for: a) one or more helper proteins; b) a self-excising element upstream of the one or more helper proteins; and c) an inducible promoter upstream of the self-excising element. In some embodiments, the self-excising element is operably linked to the inducible promoter. In some embodiments, expression of the self-excising element is driven by the inducible promoter.

In some embodiments, the inducible promoter is a tetracycline-responsive promoter element (TRE). In some embodiments, the TRE comprises Tet operator (tetO) sequence concatemers fused to a minimal promoter. In some embodiments, the minimal promoter is a human cytomegalovirus promoter. In some embodiments, the inducible promoter comprises a sequence having at least 70%, 80%, 90%, 95%, 99%, or 100% sequence identity to SEQ ID NO: 22. In some embodiments, transcription is activated from the inducible promoter upon binding of an activator. In some embodiments, the activator binds to the inducible promoter in the presence of a first triggering agent. In some embodiments, further comprising an activator. In some embodiments, the activator is operably linked to a constitutive promoter. In some embodiments, the constitutive promoter is Elalpha promoter or human cytomegalovirus promoter. In some embodiments, the E1 alpha promoter comprises at least one mutation. In some embodiments, the constitutive promoter comprises a sequence having at least 70%, 80%, 90%, 95%, 99%, or 100% sequence identity with SEQ ID NO: 20. In some embodiments, the activator is reverse tetracycline-controlled transactivator (rTA) comprising a Tet Repressor binding protein (TetR) fused to a VP16 transactivation domain. In some embodiments, the rTA comprises four mutations in the tetR DNA binding moiety. In some embodiments, the rTA comprises a sequence having at least 70%, 80%, 90%, 95%, 99%, or 100% sequence identity to SEQ ID NO: 21.

In some embodiments, the inducible promoter is bound by a repressor in the absence of a first triggering agent. In some embodiments, the inducible promoter is activated in the presence of a first triggering agent. In some embodiments, the first triggering agent binds to the repressor. In some embodiments, the repressor is a tetracycline-controlled transactivator. In some embodiments, further comprising the repressor. In some embodiments, the repressor is operably linked to a constitutive promoter. In some embodiments, further comprising a tetracycline-controlled transactivator. In some embodiments, the tetracycline-controlled transactivator is operably linked to a constitutive promoter. In some embodiments, the constitutive promoter is Elalpha promoter. In some embodiments, the E1 alpha promoter comprises at least one mutation. In some embodiments, the constitutive promoter comprises a sequence having at least 70%, 80%, 90%, 95%, 99%, or 100% sequence identity with SEQ ID NO: 20. In some embodiments, the tetracycline-controlled transactivator is unbound in the presence of a first triggering agent. In some embodiments, the tetracycline-controlled transactivator does not bind to the inducible promoter in the presence of a first triggering agent. In some embodiments, the constitutive promoter is Elalpha promoter. In some embodiments, the E1 alpha promoter comprises at least one mutation. In some embodiments, the constitutive promoter comprises a sequence having at least 70%, 80%, 90%, 95%, 99%, or 100% sequence identity with SEQ ID NO: 20. In some embodiments, transcription is activated from the inducible promoter upon binding of the first triggering agent to the repressor. In some embodiments, the repressor binds to the first triggering agent. In some embodiments, the first triggering agent is a tetracycline. In some embodiments, the tetracycline is doxycycline.

In some embodiments, wherein the inducible promoter is a cumate operator sequence. In some embodiments, the cumate operator sequence is downstream of a constitutive promoter. In some embodiments, the constitutive promoter is a human cytomegalovirus promoter. In some embodiments, wherein the inducible promoter is bound by a cymR repressor in the absence of a first triggering agent. In some embodiments, the inducible promoter is activated in the presence of a first triggering agent. In some embodiments, the first triggering agent binds to the cymR repressor. The polynucleotide construct of any one of claims X, further comprising a cymR repressor. In some embodiments, the cymR repressor is operably linked to a constitutive promoter. In some embodiments, the constitutive promoter is Elalpha promoter. In some embodiments, the E1 alpha promoter comprises at least one mutation. In some embodiments, the constitutive promoter comprises a sequence having at least 70%, 80%, 90%, 95%, 99%, or 100% sequence identity with SEQ ID NO: 20. In some embodiments, the first triggering agent is a cumate.

In some embodiments, a sequence coding for the self-excising element comprises a poly A sequence. In some embodiments, the self-excising element is a recombinase. In some embodiments, the recombinase is a site-specific recombinase. In some embodiments, the recombinase is fused to a ligand binding domain. In some embodiments, the recombinase is Cre polypeptide or flippase polypeptide. In some embodiments, the Cre polypeptide is fused to a ligand binding domain. In some embodiments, the ligand binding domain is a hormone receptor. In some embodiments, the hormone receptor is an estrogen receptor. In some embodiments, the estrogen receptor comprises a point mutation. In some embodiments, the estrogen receptor is ERT2. In some embodiments, the recombinase is a Cre-ERT2 polypeptide. In some embodiments, the self-excising element translocates to the nucleus in the presence of a second triggering agent. In some embodiments, the second triggering agent is an estrogen receptor ligand. In some embodiments, the second triggering agent is a selective estrogen receptor modulator (SERM). In some embodiments, the second triggering agent is tamoxifen. In some embodiments, the recombinase is flanked by recombination sites In some embodiments, the recombination sites are lox sites or flippase recognition target (FRT) sites. In some embodiments, the lox sites are loxP sites.

In some embodiments, the one or more adenoviral helper proteins comprise E2A and E4. In some embodiments, the one or more adenoviral helper proteins further comprises a protein tag. In some embodiments, the protein tag is a FLAG-tag. In some embodiments, the E2A is FLAG-tagged E2A. In some embodiments, the sequence coding for E2 and the sequence coding for E4 are separated by an internal ribosome entry site (IRES) or by P2A.

In some embodiments, the inducible helper construct further comprising a sequence coding for a selectable marker. In some embodiments, the selectable marker is an antibiotic resistance protein. In some embodiments, the selectable marker is a split intein linked to an N-terminus of the antibiotic resistance protein or split intein linked to a C-terminus of the antibiotic resistance protein. In some embodiments, the selectable marker is a leucine zipper linked to an N-terminus of the antibiotic resistance protein or leucine zipper linked to a C-terminus of the antibiotic resistance protein. In some embodiments, the antibiotic resistance protein is for puromycin resistance or blasticidin resistance.

In some embodiments, an inducible helper construct further comprises a sequence coding for VA RNA. In some embodiments, the sequence coding for VA RNA is a transcriptionally dead sequence. In some embodiments, the sequence coding for VA RNA comprises at least two mutations in the internal promoter. In some embodiments, expression of VA RNA is driven by a U6 promoter. The polynucleotide construct of any one of claims X, comprising upstream of the sequence coding for VA RNA gene sequence, from 5′ to 3′: a) a first part of a U6 promoter sequence; b) a first recombination site; c) a stuffer sequence; d) a second recombination site; e) a second part of a U6 promoter sequence. In some embodiments, the stuffer sequence is excisable by the recombinase. In some embodiments, the stuffer sequence comprises a sequence encoding a gene. In some embodiments, the stuffer sequence comprises a promoter. In some embodiments, the promoter is a constitutive promoter. In some embodiments, the promoter is a CMV promoter.

In some embodiments, the gene encodes a detectable marker or a selectable marker. In some embodiments, the selectable marker is an antibiotic resistance protein. In some embodiments, the selectable marker is a split intein linked to an N-terminus of the antibiotic resistance protein or split intein linked to a C-terminus of the antibiotic resistance protein. In some embodiments, the selectable marker is a leucine zipper linked to an N-terminus of the antibiotic resistance protein or leucine zipper linked to a C-terminus of the antibiotic resistance protein. In some embodiments, the selectable marker is a mammalian cell selection element. In some embodiments, the selectable marker is an auxotrophic selection element. In some embodiments, the auxotrophic selection element codes for an active protein. In some embodiments, the active protein is DHFR. In some embodiments, the auxotrophic selection coding sequence encodes an inactive protein that requires expression of a second auxotrophic selection coding sequence for activity. In some embodiments, the second auxotrophic selection coding sequence encodes for DHFR Z-Cter or DHFR Z-Nter. In some embodiments, the inactive protein comprises a DHFR Z-Nter or DHFR Z-Cter In some embodiments, the selectable marker is DHFR Z-Nter or DHFR Z-Cter. In some embodiments, the DHFR Z-Nter comprises a sequence having at least 70%, 80%, 90%, 95%, 99%, or 100% sequence identity to SEQ ID NO: 4. In some embodiments, wherein the DHFR Z-Cter comprises a sequence having at least 70%, 80%, 90%, 95%, 99%, or 100% sequence identity to SEQ ID NO: 5. In some embodiments, the antibiotic resistance protein is for puromycin resistance or blasticidin resistance. In some embodiments, the detectable marker comprises a luminescent marker or a fluorescent marker. In some embodiments, the fluorescent marker is GFP, EGFP, RFP, CFP, BFP, YFP, or mCherry. In some embodiments, the first recombination site is a first lox sequence and the second recombination site is a second lox sequence. In some embodiments, the first lox sequence is a first loxP site and the second lox sequence is a second loxP site. In some embodiments, the first recombination site is a first FRT site and the second recombination site is a second FRT site.

In some embodiments, an inducible helper construct is in a vector. In some embodiments, an inducible helper construct is in a plasmid. In some embodiments, an inducible helper construct is in a bacterial artificial chromosome or yeast artificial chromosome. In some embodiments, an inducible helper construct is a synthetic nucleic acid construct. In some embodiments, an inducible helper construct comprises a sequence having at least 70%, 80%, 90%, 95%, 99%, or 100% sequence identity to any one of SEQ ID NO: 9-SEQ ID NO: 19, SEQ ID 23-SEQ ID NO: 32, or SEQ ID NO: 35. In some embodiments, an inducible helper construct has at least 70%, 80%, 90%, 95%, 99%, or 100% sequence identity to any one of SEQ ID NO: 9-SEQ ID NO: 19, SEQ ID 23-SEQ ID NO: 32, or SEQ ID NO: 35.

In some embodiments, an inducible helper construct comprises a polynucleotide construct coding for a VA RNA, wherein a sequence coding for the VA RNA comprises at least two mutations in an internal promoter. In some embodiments, a separate polynucleotide construct codes for a VA RNA, wherein a sequence coding for the VA RNA comprises at least two mutations in an internal promoter. In some embodiments, the sequence coding for the VA RNA comprises a sequence coding for a transcriptionally dead VA RNA. In some embodiments, the sequence coding for the VA RNA comprises a deletion of from about 5-10 nucleotides in the promoter region. In some embodiments, the sequence coding for the VA RNA comprises at least one mutation. In some embodiments, the at least one mutation is in the A Box promoter region. In some embodiments, the at least one mutation is in the B Box promoter region. In some embodiments, the at least one mutation is G16A and G60A. In some embodiments, expression of the VA RNA is driven by a U6 promoter. The polynucleotide construct of any one of claims X, comprising upstream of the VA RNA gene sequence, from 5′ to 3′: a) a first part of a U6 promoter sequence; b) a first recombination site; c) a stuffer sequence; d) a second recombination site; e) a second part of a U6 promoter sequence. In some embodiments, the stuffer sequence is excisable by a recombinase. In some embodiments, the stuffer sequence comprises a sequence encoding a gene. In some embodiments, the stuffer sequence comprises a promoter. In some embodiments, the promoter is a constitutive promoter. In some embodiments, the promoter is a CMV promoter. In some embodiments, the gene encodes a detectable marker or a selectable marker. In some embodiments, the selectable marker is a mammalian cell selection element. In some embodiments, the selectable marker is an auxotrophic selection element. In some embodiments, the auxotrophic selection element codes for an active protein. In some embodiments, the active protein is DHFR. In some embodiments, the auxotrophic selection coding sequence encodes an inactive protein that requires expression of a second auxotrophic selection coding sequence for activity. In some embodiments, the second auxotrophic selection coding sequence encodes for DHFR Z-Cter or DHFR Z-Nter. In some embodiments, the inactive protein comprises a DHFR Z-Nter or DHFR Z-Cter In some embodiments, the selectable marker is DHFR Z-Nter or DHFR Z-Cter. In some embodiments, wherein the DHFR Z-Nter comprises a sequence having at least 70%, 80%, 90%, 95%, 99%, or 100% sequence identity to SEQ ID NO: 4. In some embodiments, the DHFR Z-Cter comprises a sequence having at least 70%, 80%, 90%, 95%, 99%, or 100% sequence identity to SEQ ID NO: 5. In some embodiments, the selectable marker is an antibiotic resistance protein. In some embodiments, the selectable marker is a split intein linked to an N-terminus of the antibiotic resistance protein or split intein linked to a C-terminus of the antibiotic resistance protein. In some embodiments, the selectable marker is a leucine zipper linked to an N-terminus of the antibiotic resistance protein or leucine zipper linked to a C-terminus of the antibiotic resistance protein. In some embodiments, the antibiotic resistance protein is for puromycin resistance or blasticidin resistance. In some embodiments, the detectable marker comprises a luminescent marker or a fluorescent marker. In some embodiments, the fluorescent marker is GFP, EGFP, RFP, CFP, BFP, YFP, or mCherry. In some embodiments, an inducible helper construct comprises a polynucleotide construct coding for a VA RNA or the VA RNA construct further comprising a sequence coding for a recombinase. In some embodiments, the recombinase is exogenously provided. In some embodiments, the recombinase is a site-specific recombinase. In some embodiments, the recombinase is a Cre polypeptide or a Flippase polypeptide. In some embodiments, the Cre polypeptide is fused to a ligand binding domain. In some embodiments, the ligand binding domain is a hormone receptor. In some embodiments, the hormone receptor is an estrogen receptor. In some embodiments, the estrogen receptor comprises a point mutation. In some embodiments, the estrogen receptor is ERT2. In some embodiments, the recombinase is a Cre-ERT2 polypeptide. In some embodiments, the first recombination site is a first lox sequence and the second recombination site is a second lox sequence. In some embodiments, the first lox sequence is a first loxP site and the second lox sequence is a second loxP site. In some embodiments, the first recombination site is a first FRT site and the second recombination site is a second FRT site. In some embodiments, the construct comprising the VA RNA as described herein further comprises a sequence coding for a selectable marker. In some embodiments, the selectable marker is an antibiotic resistance protein. In some embodiments, the selectable marker is a split intein linked to an N-terminus of the antibiotic resistance protein or split intein linked to a C-terminus of the antibiotic resistance protein. In some embodiments, the selectable marker is a leucine zipper linked to an N-terminus of the antibiotic resistance protein or leucine zipper linked to a C-terminus of the antibiotic resistance protein. In some embodiments, the antibiotic resistance protein is for puromycin resistance or blasticidin resistance. In some embodiments, an inducible helper construct comprises a polynucleotide construct coding for a VA RNA or the VA RNA construct is in a vector. In some embodiments, an inducible helper construct comprises a polynucleotide construct coding for a VA RNA or the VA RNA construct is in a plasmid. In some embodiments, an inducible helper construct comprises a polynucleotide construct coding for a VA RNA or the VA RNA construct is in a bacterial artificial chromosome or yeast artificial chromosome. In some embodiments, an inducible helper construct comprises a polynucleotide construct coding for a VA RNA or the VA RNA construct is a synthetic nucleic acid construct. In some embodiments, an inducible helper construct comprises a sequence having at least 70%, 80%, 90%, 95%, 99%, or 100% sequence identity to any one of SEQ ID NO: 13-SEQ ID NO: 19 or SEQ ID 23-SEQ ID NO: 2. In some embodiments, an inducible helper construct has at least 70%, 80%, 90%, 95%, 99%, or 100% sequence identity to any one of SEQ ID NO: 13-SEQ ID NO: 19 or SEQ ID 23-SEQ ID NO: 2. In some embodiments, a VA RNA construct comprises a sequence having at least 70%, 80%, 90%, 95%, 99%, or 100% sequence identity to any one of SEQ ID NO: 13-SEQ ID NO: 19 or SEQ ID 23-SEQ ID NO: 2. In some embodiments, a VA RNA construct has a sequence having at least 70%, 80%, 90%, 95%, 99%, or 100% sequence identity to any one of SEQ ID NO: 13-SEQ ID NO: 19 or SEQ ID 23-SEQ ID NO: 2.

### 6.4.3. Construct 3 (Polynucleotide Encoding a Payload)

In some embodiments, the third integrated synthetic construct comprises the coding sequence for an expressible payload and a third mammalian cell selection element. In the exemplary embodiments shown in FIG. 4, the expressible payload is under the control of a constitutive promoter. This construct can be referred to as construct 3 or payload construct, interchangeably.

In some embodiments, the expressible payload encodes a guide RNA. In certain embodiments, the guide RNA directs RNA editing. In some embodiments, the guide RNA directs CAS-mediated DNA editing. In some embodiments, the third integrated synthetic construct comprises a sequence encoding for any of the expressible payloads disclosed herein. For example, said sequence can encode for any therapeutic. For example, the therapeutic may be a transgene, a guide RNA, an antisense RNA, an oligonucleotide, an mRNA, a miRNA, a shRNA, a tRNA suppressor, a CRISPR-Cas protein, any gene editing enzyme, or any combination thereof. In some embodiments, the third integrated synthetic construct comprises sequences encoding for more than one of the expressible payloads disclosed herein.

In some embodiments, the expressible payload encodes a protein. In certain embodiments, the expressible payload is an enzyme, useful for replacement gene therapy. In some embodiments, the protein is a therapeutic antibody. In some embodiments, the protein is a vaccine immunogen. In particular embodiments, the vaccine immunogen is a viral protein.

In some embodiments, the expressible payload is a homology construct for homologous recombination.

In various embodiments, the third mammalian cell selection element is an auxotrophic selection element.

In some embodiments, the payload construct comprises a sequence having at least 70%, 80%, 90%, 95%, 99%, or 100% sequence identity to SEQ ID NO: 33. In some embodiments, the payload construct comprises a sequence having at least 70%, 80%, 90%, 95%, 99%, or 100% sequence identity to SEQ ID NO: 33, wherein SEQ ID NO: 34 in SEQ ID NO: 33 is replaced with a sequence of the payload of interest. In some embodiments, the payload construct comprises a sequence of a payload flanked by ITR sequences. In some embodiments, expression of the sequence of the payload is driven by a constitutive promoter. In some embodiments, the constitutive promoter and sequence of the payload are flanked by ITR sequences. In some embodiments, the sequence of the payload comprises a polynucleotide sequence coding for a gene. In some embodiments, the gene codes for a selectable marker or detectable marker. In some embodiments, the gene codes for a therapeutic polypeptide or transgene. In some embodiments, the sequence of the payload comprises a polynucleotide sequence coding for a therapeutic polynucleotide. In some embodiments, the therapeutic polynucleotide is a tRNA suppressor or a guide RNA. In some embodiments, the guide RNA is a polyribonucleotide capable of binding to a protein. In some embodiments, the protein is nuclease. In some embodiments, the protein is a Cas protein, an ADAR protein, or an ADAT protein. In some embodiments, the Cas protein is catalytically inactive Cas protein. In some embodiments, the payload construct is stably integrated into the genome of the cell. In some embodiments, a plurality of the payload construct are stably integrated into the genome of the cell. In some embodiments, the plurality of the payload constructs are separately stably integrated into the genome of the cell. In some embodiments, the payload construct further comprises a sequence coding for a selectable marker or detectable marker outside of the ITR sequences. In some embodiments, the selectable marker is a mammalian cell selection element. In some embodiments, the selectable marker is an auxotrophic selection element. In some embodiments, the auxotrophic selection element codes for an active protein. In some embodiments, the active protein is DHFR. In some embodiments, the auxotrophic selection coding sequence encodes an inactive protein that requires expression of a second auxotrophic selection coding sequence for activity. In some embodiments, the second auxotrophic selection coding sequence encodes for DHFR Z-Cter or DHFR Z-Nter. In some embodiments, the inactive protein comprises a DHFR Z-Nter or DHFR Z-Cter In some embodiments, the selectable marker is DHFR Z-Nter or DHFR Z-Cter. The polynucleotide construct of any one of claims 2-6, wherein the DHFR Z-Nter comprises a sequence having at least 70%, 80%, 90%, 95%, 99%, or 100% sequence identity to SEQ ID NO: 4. The polynucleotide construct of any one of claims 2-6, wherein the DHFR Z-Cter comprises a sequence having at least 70%, 80%, 90%, 95%, 99%, or 100% sequence identity to SEQ ID NO: 5. In some embodiments, the selectable marker is an antibiotic resistance protein. In some embodiments, the selectable marker outside of the ITR sequences is a split intein linked to an N-terminus of the antibiotic resistance protein or split intein linked to a C-terminus of the antibiotic resistance protein. In some embodiments, the selectable marker outside of the ITR sequences is a leucine zipper linked to an N-terminus of the antibiotic resistance protein or leucine zipper linked to a C-terminus of the antibiotic resistance protein. In some embodiments, the antibiotic resistance protein is for puromycin resistance or blasticidin resistance.

### 6.4.4. Host Production Cell

The plurality of synthetic nucleic acid constructs are integrated into the genome of a production host cell. In some embodiments, the production cell is an insect cell. In some embodiments, the production cell is a mammalian cell.

In typical embodiments, the production host cell is a mammalian cell line that expresses adenovirus E1A and E1B. In particular embodiments, the cell is a human embryonic kidney (HEK) 293 cell line or derivatives thereof (HEK293T cells, HEK293F cells), a human HeLa cell line that expresses E1A and E1B, a Chinese hamster ovary (CHO) cell line that expresses E1A and E1B, or a Vero cell that expresses adenovirus E1A and E1B. In particular embodiments, the host cell is a HEK293 cell line.

In certain embodiments, the host cell is DHFR null. In specific embodiments, the host cell is a DHFR null HEK293 cell.

In some embodiments, the HEK293 cell expresses AAV E1A and E1B. In the presence of doxycycline and tamoxifen, the ER2 Cre is excised from the first integrated synthetic construct, thereby permitting expression of AAV E2A and E4. The self-excised ER2 Cre recombines by virtue of the lox sites flanking the EGFP cassette in the second integrated synthetic construct, thereby removing the EGFP segment from the second spacer element in the integrated second synthetic construct. As such, any cells comprising only the second integrated synthetic construct will be EGFP signal positive whereas cells comprising both the first and second integrated synthetic constructs will be EGFP signal negative, following the addition of the triggering agents. Absence of EGFP signal indicates successful transfection of both the first and second integrated synthetic constructs in a cell. This is further ensured by antibiotic resistance selection, e.g., blasticidin resistance.

Additionally, removal of the EGFP cassette provides for the functional expression of Rep and Cap proteins, which can be linked to a first DHFR selection element, e.g., Z-Cter DHFR. The Z-Cter DHFR is capable of associating with a second DHFR selection element, e.g., Z-Nter DHFR, present in the third integrated synthetic construct to form an active molecule that allows the cell to survive in a selection medium, e.g., HT lacking media selection.

In some embodiments, the third integrated synthetic construct comprises a payload. The payload can be a guide RNA (FIGS. 4 and 5B), an HDR homology region, or a gene of interest.

In sum, a preferred embodiment of this system requires only one antibiotic resistance marker, and two split auxotrophic constructs for selection of all three plasmids, each being transformed just once into the DHFR knockout strain—producing a master cell line for virion production which can be stored and then utilized for scaled-up production without further transformations. This approach provides inducible control over expression of the Rep/Cap products avoiding the toxicity typically associated with Rep/Cap production and also avoids selection with multiple antibiotics, which is not preferred for therapeutic products. Both overexpression of Rep/Cap and selection with multiple antibiotics can be toxic and result in diminished virion yield. The transformed cells can be frozen for storage and thawed for subsequent applications.

### 6.4.5. Payloads

Disclosed herein are payloads that may be encoded for by polynucleotide construct 3, which encodes for a payload. This third polynucleotide is referred to herein as a “payload construct” or “therapeutic payload.” Thus, disclosed herein are stable mammalian cell lines that encapsidate a payload. The payload may be an expressible payload. The polynucleotide may encode for any therapeutic. For example, the therapeutic may be a transgene, a guide RNA, an antisense RNA, an oligonucleotide, an mRNA, a miRNA, a shRNA, a tRNA suppressor, a CRISPR-Cas protein, any gene editing enzyme, or any combination thereof. In some embodiments, the stable mammalian cell lines disclosed herein can conditionally produce rAAV virions that encapsidate more than one payload. Any combination of payloads disclosed herein is contemplated.

### 6.4.6. Split Auxotrophic Selection

Maintaining constructs stably in the cellular genome requires selective pressure.

Typically, each integrated nucleic acid construct comprises a mammalian cell selection element. In some embodiments, the stable cell line comprises three integrated nucleic acid constructs, wherein the first nucleic acid construct comprises a first mammalian cell selection element, the second nucleic acid construct comprises a second mammalian cell selection element, and the third nucleic acid construct comprises a third mammalian cell selection element.

FIG. 5A depicts an exemplary split auxotrophic selection system that permits stable retention of two integrated nucleic acid constructs under a single selective pressure. One construct encodes the N-terminal fragment of mammalian dihydrofolate reductase (DHFR) fused to a leucine zipper peptide (“Nter-DHFR”). This N-terminal fragment is enzymatically nonfunctional. The other construct encodes the C-terminal fragment of DHFR fused to a leucine zipper peptide (“Cter-DHFR”). This C-terminal fragment is enzymatically nonfunctional. When both fragments are concurrently expressed in the cell, a functional DHFR enzyme complex is formed through association of the leucine zipper peptides. Both constructs can be stably retained in the genome of a DHFR null cell by growth in a medium lacking hypoxanthine and thymidine.

FIG. 5B shows an exemplary deployment of this split auxotrophic selection design in the multi-construct system of FIG. 1 in its pre-triggered state. In this embodiment, the split auxotrophic selection elements are deployed on constructs 1 and 3. A separate exemplary antibiotic selection element, blasticidin resistance, is deployed on construct 2. This results in the ability to stably maintain all three constructs in the mammalian cell line using a single antibiotic, culturing in medium with blasticidin, lacking thymidine and hypoxanthine. In some embodiments, the construct 2 further comprising a sequence coding for VA RNA as described herein. In some embodiments, the VA RNA is a mutated VA RNA. In some embodiments, the VA RNA is transcriptionally dead VA RNA. In some embodiments, the VA RNA is under the control of a U6 promoter. In some embodiments, the U6 promoter is a conditionally active. In some embodiments, the U6 promoter comprises an interrupting sequence that is capable of being floxed upon addition a triggering agent (e.g., the triggering agent induces the expression of a recombinase as described herein).

In some embodiments, the first nucleic acid construct comprises a first mammalian cell selection element, and the first mammalian cell selection element is a first auxotrophic selection element. In certain embodiments, the first auxotrophic selection element encodes an active protein. In particular embodiments, the first auxotrophic selection element is DHFR. In some embodiments, the first auxotrophic selection coding sequence encodes an inactive protein that requires expression of a second auxotrophic selection coding sequence for activity. In certain embodiments, the first auxotrophic selection element encodes Z-Cter-DHFR (SEQ ID NO: 5).

In various embodiments, the second nucleic acid construct comprises a second mammalian cell selection element, and the second mammalian cell selection element encodes antibiotic resistance. In particular embodiments, the antibiotic resistance gene is a blasticidin resistance gene.

In various embodiments, the third nucleic acid construct comprises a third mammalian cell selection element. In some embodiments, the third mammalian cell selection element is a second auxotrophic selection element. In certain embodiments, the second auxotrophic selection element encodes an active protein. In particular embodiments, the second auxotrophic selection element is DHFR. In some embodiments, the second auxotrophic selection coding sequence encodes an inactive protein that requires expression of a first auxotrophic selection coding sequence for activity. In certain embodiments, the second auxotrophic selection element encodes Z-Nter-DHFR (SEQ ID NO:4).

In various embodiments, the stable mammalian cell line can be propagated in growth media lacking hypoxanthine and thymidine.

### 6.4.7. Complete System in Detail

The first integrated synthetic construct comprises an intervening spacer sequence inserted into the coding sequence of AAV2 Rep protein. The intervening spacer sequence comprises an enhanced green fluorescent protein (EGFP) and a rabbit beta globin (RBG) polyadenylation (polyA) signal, flanked by two lox sites, are inserted into an RBG intron. The RBG intron includes the 5′ splice site (5′SS) and the 3′ splice site (3′SS) (as shown in FIGS. 3A-3B). The RBG intron is inserted downstream of the Rep endogenous P5 and P19 promoters and interrupt the Rep coding sequence. This design blocks the expression of Rep proteins generated by both P5 and P19 promoters. The EGFP serves as a visual indicator of successful integration and to monitor Cre mediated excision, and could be replaced by any suitable marker. For instance, loss of EGFP expression indicates successful Cre-mediated genomic recombination (See, FIG. 3B). Current approaches rely on inserting the EGFP and the polyA within an intron without duplication of 3′ splice site (3′SS). If there is readthrough after polyA, the 5′SS can combine with the native 3′SS, thus removing the entire RBG intron and as a result, commencing Rep expression. By contrast, the design as described herein includes an additional 3′SS upstream of the EGFP, which solves the problem of undesired Rep expression. The present design provides that if there is readthrough, the construct allows splicing of 5′SS to the upstream 3′SS. Without being bound to any theories, the additional 3′SS which is nearest to the 5′SS is preferred since it is the same 3′SS as the downstream one and the two 3′SS are of equal strength. As such, all Rep proteins will be produced fused to the EGFP protein and then terminate. In an event where the Rep proteins do not terminate after EGFP, they will continue to produce codons coded by the rest of the RBG intro, thereby making a non-functional Rep protein. This approach prevents overexpression of Rep proteins which may have inhibitory effects on adenovirus and cell growth, thereby reducing toxicity of the recombinant AAV (rAAV) construct.

As described herein, expression of functional Rep protein is induced only in the presence of a first expression triggering agent, e.g., the addition of doxycycline which results in the production of Cre. In the presence of Cre, the intervening spacer is excised thereby resuming intact coding sequencing of the Rep protein. This approach provides controlled and inducible Rep expression.

This is driven by the second integrated synthetic construct, which comprises an estrogen inducible Cre (ER2 Cre) gene and adenoviral helper genes, E2A and E4orf6 (E4) (See, FIGS. 1, 2A-2B, 3A-3B, and 6).

In certain embodiments, the third integrated synthetic construct (“construct three”) comprises a polynucleotide flanked by AAV inverted terminal repeats (ITRs, shown by brackets in FIG. 1, FIG. 4, FIG. 5B, and FIG. 6). In certain embodiments, the third integrated construct further comprises a component of a split auxotrophic selection, as described above in Section 4.4.5. In particular embodiments, the component of the split auxotrophic selection comprises a first enzymatically nonfunctional dihydrofolate reductase (DHFR) fragment fused to a leucine zipper. Binding with a second DHFR fragment also fused to a leucine zipper produces an active complex, and allows selection for cells expressing both the first and the third integrated synthetic constructs. The construct three polynucleotide can comprise any payload including at least a guide RNA, a gene of interest, a transgene, an HDR homology region, a minigene or a therapeutic polynucleotide. This approach requires only a single auxotrophic selection agent, and a single antibiotic selection agent to be present in the cell culture medium to maintain all of the plurality of synthetic nucleic acid constructs stably within the nuclear genome of the cells. This approach also avoids multiple antibiotic resistance selection, which may be undesirable for downstream applications, e.g., gene therapy.

In one aspect, provided herein is a stable mammalian cell line, wherein the cells are capable of conditionally producing recombinant AAV (rAAV) virions within which are packaged an expressible payload; and production of virions is not conditioned on the presence of an episome within the cell.

In various embodiments, expression of AAV rep and cap proteins is conditional. In some embodiments, expression of AAV rep and cap proteins is conditioned on addition of at least a first expression triggering agent to the cell culture medium. In some embodiments, expression of AAV Rep and Cap proteins is conditioned on addition of a first expression triggering agent and a second expression triggering agent to the cell culture medium.

In some embodiments, the cells do not express cytotoxic levels of Rep protein prior to addition of the at least a first expression triggering agent to the cell culture medium. In some embodiments, the cells do not express cytostatic levels of Rep protein prior to addition of the at least first expression triggering agent to the cell culture medium.

In some embodiments, the average concentration of Rep protein within the cells is less than between 1-99%, 10-90%, 20-80%, 30-70%, 40-60% prior to addition of the at least first expression triggering agent to the cell culture medium. In some embodiments, the average concentration of Rep protein within the cells is less than about 1%, 5%, 10%, 15%, 20%, 25%, 30%, 35%, 40%, 45%, 50%, 55%, 60%, 65%, 70%, 75%, 80%, 85%, 90%, 95%, or 99% prior to addition of the at least first expression triggering agent to the cell culture medium.

In various embodiments, expression of Rep and Cap proteins becomes constitutive after addition of the at least first expression triggering agent to the cell culture medium. The stable cell lines include those wherein expression of adenoviral helper proteins is conditional. In some embodiments, expression of adenoviral helper proteins is conditioned on addition of at least a first expression triggering agent to the cell culture medium. In some embodiments, expression of adenoviral helper proteins is conditioned on addition of a first expression triggering agent and a second expression triggering agent to the cell culture medium. In some embodiments, continued expression of adenoviral helper proteins following triggering of expression requires presence of only the first expression triggering agent in the cell culture medium.

In some embodiments, the adenoviral helper proteins include E2A and E4.

In some embodiments, the first expression triggering agent is a tetracycline. In some embodiments, the tetracycline is doxycycline.

In some embodiments, the second expression triggering agent is an estrogen receptor ligand. In some embodiments, the estrogen receptor ligand is a selective estrogen receptor modulator (SERM). In some embodiments, the estrogen receptor ligand is tamoxifen.

In some embodiments, expression of the payload is not conditioned on addition of an expression triggering agent to the cell culture medium.

In various embodiments, the nuclear genome of the cell comprises a plurality of integrated synthetic nucleic acid constructs. In some embodiments, the nuclear genome of the cell comprises two integrated synthetic constructs. In some embodiments, the nuclear genome of the cell comprises three integrated synthetic constructs. In some embodiments, each of the plurality of synthetic nucleic acid constructs is separately integrated into the nuclear genome of the cell.

In some embodiments, only a single non-auxotrophic selection agent is required to be present in the cell culture medium to maintain all of the plurality of synthetic nucleic acid constructs stably within the nuclear genome of the cells.

In some embodiments, the first integrated synthetic construct comprises conditionally expressible AAV Rep and Cap coding sequences; the second integrated synthetic construct comprises a conditionally expressible Cre coding sequence and conditionally expressible adenoviral helper protein coding sequences; and the third integrated synthetic construct comprises expressible coding sequences for the payload.

In some embodiments, the first integrated construct comprises a Rep coding sequence interrupted by an intervening spacer. In some embodiments, the intervening spacer comprises, from 5′ to 3′, a first spacer, a second spacer and a third spacer. In some embodiments, the intervening spacer comprises nucleic acid sequences of a rabbit beta globin (RBG) intron and a rabbit beta globin (RBG) poly A. In some embodiments, the first spacer comprises a nucleic acid sequence of at least 80% identity to SEQ ID NO: 1. In some embodiments, the first spacer comprises a 5′ splice site (5′SS) 5′ to the first spacer. In some embodiments, the second spacer comprises a nucleic acid sequence of at least 80% identity to SEQ ID NO: 2. In some embodiments, the second spacer comprises, from 5′ to 3′ a first lox site, an enhanced green fluorescent protein (EGFP), the RBG polyA sequence, and a second lox site. In some embodiments, the second spacer further comprises a first 3′ splice site (3′SS) flanked by the first lox site and the EGFP. In some embodiments, the third spacer comprises a nucleic acid sequence of at least 80% identity to SEQ ID NO: 3. In some embodiments, the third spacer further comprises a second 3′ splice site (3′SS) 3′ to the third spacer.

In some embodiments, the Rep coding sequence comprises a polynucleotide sequence operatively linked to an endogenous P5 promoter. In some embodiments, the Rep coding sequence comprises a polynucleotide sequence operatively linked to an endogenous P19 promoter. In some embodiments, the intervening spacer is inserted into the Rep coding sequence at a position downstream of the P19 promoter. In some embodiments, the intervening spacer is inserted into the Rep coding sequence at a position in frame with the protein produced from activation of the P5 promoter and the P19 promoter. In some embodiments, wherein the Rep coding sequence is 5′ to the Cap coding sequence. In some embodiments, the Cap coding sequence is operatively linked to an endogenous P40 promoter.

In some embodiments, the second integrated construct comprises, from 5′ to 3′, a Cre coding sequence and a first polyA sequence, adenoviral helper protein coding sequences and a second polyA sequence, a first expression triggering agent responsive element, and an antibiotic selection element. In some embodiments, the Cre coding sequence is flanked by a first lox site and a second lox site. In some embodiments, the Cre coding sequence is operatively linked to an inducible promoter. In some embodiments, the inducible promoter comprises a plurality of tetracycline (Tet) operator elements capable of binding to a Tet responsive activator protein in the presence of a first expression triggering agent. In some embodiments, the inducible promoter comprises a plurality of Tet operator elements capable of binding to a Tet responsive activator protein in the presence of a first expression triggering agent and a second expression triggering agent responsive element. In some embodiments, the adenoviral helper protein coding sequences comprise E2A and E4 sequences. In some embodiments, the first expression triggering agent responsive element is operatively linked to a CMV promoter. In some embodiments, the first expression triggering agent responsive element comprises the Tet responsive activator protein (Tet-on-3G). In some embodiments, the antibiotic selection element is blasticidin resistance.

In some embodiments, the third integrated synthetic construct comprises a coding sequence for the expressible payload and a first element of an auxotrophic selection agent and the first integrated synthetic construct comprises coding sequences for a second element of the auxotrophic selection agent. In some embodiments, the first element of a auxotrophic selection agent comprises a first dihydrofolate reductase (DHFR) selectable marker (SEQ ID NO: 4). In some embodiments, the first DHFR comprises a leucine zipper (Nter). In some embodiments, the second element of the auxotrophic selection agent comprises a second DHFR (SEQ ID NO: 5). In some embodiments, the second DHFR comprises a leucine zipper (Cter). In some embodiments, the DHFR selection comprises the ability to grow in media lacking hypoxantine-thymidine.

In some embodiments, the mammalian cell line is selected from the group consisting of a human embryonic kidney (HEK) 293 cell line, a human HeLa cell line, and a Chinese hamster ovary (CHO) cell line. In some embodiments, the mammalian cell line is a HEK293 cell line. In some embodiments, the mammalian cell line expresses adenovirus helper functions E1A and E1B.

### A. The Stable Mammalian Cell or Cell Line

As described herein, the stable mammalian cell or cell line can be a human derived cell or cell line such as a human embryonic kidney (HEK) 293 cell line or a human HeLa cell line, or a mammalian cell or cell line such as Chinese hamster ovary (CHO) cell line. In some embodiments, the mammalian cell line is a HEK293 cell line. In some embodiments, the mammalian cell line expresses adenovirus helper functions E1A and E1B.

### B. The First Integrated Synthetic Construct

Shown are exemplary designs of the first integrated synthetic construct (FIGS. 1, 3B and 6).

As described in FIGS. 3A-3B, the first integrated synthetic construct comprises a Rep coding sequence 5′ of a Cap coding sequence. The Rep coding sequence is interrupted by an intervening spacer. In some embodiments, the first integrated synthetic construct further comprises a selection element such as an auxotrophic selection marker (FIG. 5B). In some embodiments, the selection element is a partial or a second element of the non-auxotrophic selection marker. The intervening spacer comprises a rabbit beta globin (RBG) intron, which is modified by duplicating the RBG 3′ splice site (3′SS) upstream to an enhanced green fluorescent protein (EGFP) cassette within the intron. The EGFP cassette is cloned immediately downstream of this duplicated splice site followed by a rabbit beta globin polyadenylation signal. This entire modification (3′SS, EGFP and polyA) is flanked by two lox sites so that the module can be removed upon Cre expression. The modified rabbit beta globin intron (the intervening spacer sequence) is inserted into the coding sequence of AAV2 Rep protein. The point of insertion is downstream of P19 promoter, away from any known regulatory elements. It is also in frame with the proteins produced from P5 and P19 protein so that EGFP expression can be visualized. Cap genes from any AAV serotype are cloned downstream of the AAV2 Rep cassette. The Cap genes are driven by their endogenous P40 promoter. In the absence of Cre, the 5′ splice site (5′SS) gets spliced to the upstream of 3′SS, so the EGFP becomes the terminal exon and transcription terminates at the beta globin polyadenylation signal. Thus, the expression of all Rep proteins either from the P5 or P19 promoter is prematurely terminated. Since expression of the P40 promoter is dependent on the presence of the Rep proteins, the P40 promoter is silent and there is no expression of Cap proteins. Upon Cre expression from a second integrated synthetic construct, the entire second spacer element (except for the left lox site is excised from the beta globin intron. The 5′SS now splices with the native 3′SS site and expression of all Rep proteins commences. Rep expression activates the P40 proteins and Cap proteins are therefore also expressed.

### C. The Second Integrated Synthetic Construct

Shown are exemplary designs of the second integrated synthetic construct (FIGS. 2A-2C).

As shown in FIG. 2A, the second integrated synthetic construct comprising, from 5′ to 3′, a Cre coding sequence and a first polyA sequence, adenoviral helper protein coding sequences and a second polyA sequence, a first expression triggering agent responsive element, and an antibiotic selection element. The Cre coding sequence is flanked by a first lox site and a second lox site, and is operatively linked to an inducible promoter. The inducible promoter comprises a plurality of tetracycline (Tet) operator elements capable of binding to a Tet responsive activator protein in the presence of a first expression triggering agent, e.g., doxycycline or tetracycline. In some embodiments, the inducible promoter comprises a plurality of tetracycline (Tet) operator elements capable of binding to a Tet responsive activator protein in the presence of a first expression triggering agent and a second expression triggering agent responsive element, e.g., tamoxifen. The first expression triggering element may comprise a Tet responsive activator protein (Tet-on-3G) and is operatively linked to a CMV promoter. The antibiotic selection element can be blasticidin resistance (FIG. 5B). The optional insert shown in FIG. 2C provides for inducible production of VA-RNA, which are short non-coding transcripts essential for Adenovirus replication. In this construct, an alternative insert to construct 2, includes a Cre inducible U6 promoter that drives the expression of transcriptionally dead mutants of VA RNA1 (a preferred embodiment is a double point mutant G16A-G60A). The U6 promoter is split into 2 parts separated by a Lox flanked stuffer sequence. The U6 promoter is inactive because of the presence of the stuffer sequence. Cre mediated excision of the stuffer activates the U6 promoter which then drives the expression of VA RNA. Other embodiments may provide for alternative sources of VA-RNA.

In some embodiments, the Cre coding sequencing is an estrogen inducible Cre that has a strong polyadenylation signal (stop signal) at its 3′ end. Following this is a bicistronic E2A, E4orf6 cassette. The plasmid also has a constitutive promoter (CMV) which drives the expression of the Tet responsive activator protein (Tet-on 3G).

In the off state when doxycycline (Dox) is absent, the Tet-on 3G cannot bind to the Tet operator elements in the Tet-regulatable promoter so the promoter is not active. Estrogen responsive Cre is used instead of simple Cre to counteract the basal or leaky expression of the Tet-regulatable promoter. In the off state if there is leaky expression of Cre gene, the expressed Cre protein will be held inactive in the cytoplasm. The strong polyadenylation signal, 3′ of the cre gene will prevent basal expression of adenoviral helper genes, E2A and E4. To induce expression, doxycycline and tamoxifen are added to the cell culture (FIG. 6). Doxycycline will bind to the Tet-on 3G protein and this will promote binding of the Tet-on 3G to the tet operator elements in the Tet-regulatable promoter. This will trigger the activation of the promoter. ER2 Cre will be expressed at high levels and tamoxifen will bring the Cre to the nucleus.

### D. The Third Integrated Synthetic Construct

Shown are exemplary designs of the third integrated synthetic construct (FIGS. 1, 4, 5B and 6). As described in FIGS. 4 and 6, the third integrated synthetic construct comprises coding sequences for an expressible payload, and/or a guide RNA, and a first element of a non-auxotrophic selection agent capable of binding to a partial or a second element of the non-auxotrophic selection agent in the first integrated synthetic construct. In some embodiments, the first element of a non-auxotrophic selection agent comprises a first dihydrofolate reductase (DHFR) selectable marker (SEQ ID NO: 4). The first DHFR may comprise a leucine zipper (Nter). In some embodiments, the second element of the non-auxotrophic selection agent comprises a second DHFR (SEQ ID NO: 5). The second DHFR may comprise a leucine zipper (Cter). In some embodiments, the DHFR selection comprises hypoxantine-thymidine selection. In some embodiments, re-association of the first and second DHFR selection markers allows for selection of a mammalian cell expression both the first integrated synthetic construct and the third integrated synthetic construct.

In some embodiments, a cell comprises two constructs (any combination of Rep/Cap construct, inducible helper construct, and the payload construct). In some embodiments, a cell comprises the Rep/Cap construct and the inducible helper construct. In some embodiments, the cell, the inducible helper construct comprises a VA RNA construct as described herein. In some embodiments, cell further comprises the VA RNA construct.

In some embodiments, a cell comprises all three constructs (Rep/Cap construct, inducible helper construct, and the payload construct). In some embodiments, the cell, the inducible helper construct comprises a VA RNA construct as described herein. In some embodiments, cell further comprises the VA RNA construct. In some embodiments, this cell is capable of producing an rAAV virion upon addition of at least one triggering agent. In some embodiments, the rAAV virion comprising the capsid protein and the payload nucleic acid sequence have an infectivity of no less than 50%, 60%, 70%, 80%, 90%, 95%, or 99% at an MOI of 1×10⁵ vg/target cell or less. In some embodiments, the rAAV virions have an increased infectivity compared rAAV virions produced by an otherwise comparable the population of cells capable of producing rAAV virions upon transient transfection at the same MOI. In some embodiments, the rAAV virions have at least 1%, 5%, 10%, 15%, 20%, 30%, 40%, or 50% greater infectivity compared rAAV virions produced by an otherwise comparable the population of cells capable of producing rAAV virions upon transient transfection at the same MOI. In some embodiments, the rAAV virions have at least 50%, 60%, 70%, 80%, 90%, 95%, 97%, 98%, 99%, or 100% infectivity as compared to AAV virions produced by a cell having wildtype AAV at the same MOI. In some embodiments, the rAAV virions have at least 50%, 60%, 70%, 80%, 90%, 95%, 97%, 98%, 99%, or 100% infectivity as compared to AAV virions at the same MOI. In some embodiments, the AAV virions are wildtype AAV virions produced by a cell having wildtype AAV. In some embodiments, the MOI is 1×10¹, 1×10², 2×10³, 5×10⁴, or 1×10⁵ vg/target cell. In some embodiments, the MOI is selected from a range of 1×10¹ to 1×10⁵ vg/target cell. In some embodiments, the cell is conditionally capable of producing rAAV virions having a payload encapsidation ratio of no less than 0.5, 0.6, 0.7, 0.8, 0.9, 0.95, 0.97, or 0.99. In some embodiments, the rAAV virions have a payload encapsidation ratio of no less than 0.5, 0.6, 0.7, 0.8, 0.9, 0.95, 0.97, or 0.99 prior to purification. In some embodiments, the rAAV virions have a concentration of greater than 1×10¹¹ or no less than 5×10¹¹, 1×10¹², 5×10¹², 1×10¹³ or 1×10¹⁴ viral genomes per milliliter prior to purification. In some embodiments, the cell is capable of producing rAAV virions comprising the payload nucleic acid sequence at a titer of greater than 1×10¹¹ or no less than 5×10¹¹, 1×10¹², 5×10¹², 1×10¹³ or 1×10¹⁴ viral genomes per milliliter. In some embodiments, the cell is capable of producing rAAV virions comprising the payload nucleic acid sequence at a concentration of greater than 1×10¹¹ or no less than 5×10¹¹, 1×10¹², 5×10¹², 1×10¹³ or 1×10¹⁴ viral genomes per milliliter prior to purification. In some embodiments, this cell is expanded to produce a population of cells. In some embodiments, the population of cells produces a stable cell line as described herein. In some embodiments, this cell is passaged at least three times. In some embodiments, this cell can be passaged up to 60 times. In some embodiments, this cell can be passage more than 60 times. In some embodiments, the cell maintains the ability to be conditionally induced after each passage.

### 6.5. Cell Comprising a Construct

In some embodiments, a cell comprise one construct (Rep/Cap construct, inducible helper construct, and the payload construct). In some embodiments, the one construct is stably integrated into the genome of the cell. In some embodiments, a plurality of the one construct is stably integrated into the genome of the cell. In some embodiments, a cell comprises two constructs (any combination of Rep/Cap construct, inducible helper construct, and the payload construct). In some embodiments, the two constructs are stably integrated into the genome of the cell. In some embodiments, the two constructs are separately stably integrated into the genome of the cell. In some embodiments, a plurality of the two constructs are stably integrated into the genome of the cell. In some embodiments, a plurality of the two constructs are separately stably integrated into the genome of the cell. In some embodiments, a cell comprises the Rep/Cap construct and the inducible helper construct. In some embodiments, the cell, the inducible helper construct comprises a VA RNA construct as described herein. In some embodiments, cell further comprises the VA RNA construct as described herein. In some embodiments, the VA RNA construct is stably integrated into the genome of the cell.

In some embodiments, a cell comprises all three constructs (Rep/Cap construct, inducible helper construct, and the payload construct). In some embodiments, the three constructs are stably integrated into the genome of the cell. In some embodiments, the three constructs are separately stably integrated into the genome of the cell. In some embodiments, a plurality of the three constructs are stably integrated into the genome of the cell. In some embodiments, a plurality of the three constructs are separately stably integrated into the genome of the cell. In some embodiments, the cell, the inducible helper construct comprises a VA RNA construct as described herein. In some embodiments, cell further comprises the VA RNA construct.

In some embodiments, a VA RNA construct is a polynucleotide construct coding for a VA RNA, wherein a sequence coding for the VA RNA comprises at least two mutations in an internal promoter. In some embodiments, the sequence coding for the VA RNA comprises a sequence coding for a transcriptionally dead VA RNA. In some embodiments, the sequence coding for the VA RNA comprises a deletion of from about 5-10 nucleotides in the promoter region. In some embodiments, the sequence coding for the VA RNA comprises at least one mutation. In some embodiments, the at least one mutation is in the A Box promoter region. In some embodiments, the at least one mutation is in the B Box promoter region. In some embodiments, the at least one mutation is G16A and G60A. In some embodiments, expression of the VA RNA is driven by a U6 promoter. The polynucleotide construct of any one of claims X, comprising upstream of the VA RNA gene sequence, from 5′ to 3′: a) a first part of a U6 promoter sequence; b) a first recombination site; c) a stuffer sequence; d) a second recombination site; e) a second part of a U6 promoter sequence. In some embodiments, the stuffer sequence is excisable by a recombinase. In some embodiments, the stuffer sequence comprises a sequence encoding a gene. In some embodiments, the stuffer sequence comprises a promoter. In some embodiments, the promoter is a constitutive promoter. In some embodiments, the promoter is a CMV promoter. In some embodiments, the gene encodes a detectable marker or a selectable marker. In some embodiments, the selectable marker is a mammalian cell selection element. In some embodiments, the selectable marker is an auxotrophic selection element. In some embodiments, the auxotrophic selection element codes for an active protein. In some embodiments, the active protein is DHFR. In some embodiments, the auxotrophic selection coding sequence encodes an inactive protein that requires expression of a second auxotrophic selection coding sequence for activity. In some embodiments, the second auxotrophic selection coding sequence encodes for DHFR Z-Cter or DHFR Z-Nter. In some embodiments, the inactive protein comprises a DHFR Z-Nter or DHFR Z-Cter In some embodiments, the selectable marker is DHFR Z-Nter or DHFR Z-Cter. In some embodiments, the DHFR Z-Nter comprises a sequence having at least 70%, 80%, 90%, 95%, 99%, or 100% sequence identity to SEQ ID NO: 4. In some embodiments, the DHFR Z-Cter comprises a sequence having at least 70%, 80%, 90%, 95%, 99%, or 100% sequence identity to SEQ ID NO: 5. In some embodiments, the selectable marker is an antibiotic resistance protein. In some embodiments, the selectable marker is a split intein linked to an N-terminus of the antibiotic resistance protein or split intein linked to a C-terminus of the antibiotic resistance protein. In some embodiments, the selectable marker is a leucine zipper linked to an N-terminus of the antibiotic resistance protein or leucine zipper linked to a C-terminus of the antibiotic resistance protein. In some embodiments, the antibiotic resistance protein is for puromycin resistance or blasticidin resistance. In some embodiments, the detectable marker comprises a luminescent marker or a fluorescent marker. In some embodiments, the fluorescent marker is GFP, EGFP, RFP, CFP, BFP, YFP, or mCherry. In some embodiments, the VA RNA construct further comprising a sequence coding for a recombinase. In some embodiments, the recombinase is exogenously provided. In some embodiments, the recombinase is a site-specific recombinase. The polynucleotide construct of any one of claims, wherein the recombinase is a Cre polypeptide or a Flippase polypeptide. In some embodiments, the Cre polypeptide is fused to a ligand binding domain. In some embodiments, the ligand binding domain is a hormone receptor. In some embodiments, the hormone receptor is an estrogen receptor. In some embodiments, the estrogen receptor comprises a point mutation. In some embodiments, the estrogen receptor is ERT2. The polynucleotide construct of any one claims X, wherein the recombinase is a Cre-ERT2 polypeptide. In some embodiments, the first recombination site is a first lox sequence and the second recombination site is a second lox sequence. In some embodiments, the first lox sequence is a first loxP site and the second lox sequence is a second loxP site. In some embodiments, the first recombination site is a first FRT site and the second recombination site is a second FRT site. The polynucleotide construct of any one of claims X, further comprising a sequence coding for a selectable marker. In some embodiments, the selectable marker is an antibiotic resistance protein. In some embodiments, the selectable marker is a split intein linked to an N-terminus of the antibiotic resistance protein or split intein linked to a C-terminus of the antibiotic resistance protein. In some embodiments, the selectable marker is a leucine zipper linked to an N-terminus of the antibiotic resistance protein or leucine zipper linked to a C-terminus of the antibiotic resistance protein. In some embodiments, the antibiotic resistance protein is for puromycin resistance or blasticidin resistance. In some embodiments, the VA RNA construct comprises a sequence having at least 70%, 80%, 90%, 95%, 99%, or 100% sequence identity to any one of SEQ ID NO: 13-SEQ ID NO: 19 or SEQ ID 23-SEQ ID NO: 2. In some embodiments, VA RNA construct has at least 70%, 80%, 90%, 95%, 99%, or 100% sequence identity to any one of SEQ ID NO: 13-SEQ ID NO: 19 or SEQ ID 23-SEQ ID NO: 26.

In some embodiments, the cell is a mammalian cell or insect cell. In some embodiments, the cell is a HEK293 cell, HeLa cell, CHO cell, or SF9 cell. In some embodiments, the cell expresses E1A protein and E1B protein. In some embodiments, the cell further comprising a payload construct. In some embodiments, the payload construct comprises a sequence having at least 70%, 80%, 90%, 95%, 99%, or 100% sequence identity to SEQ ID NO: 33. In some embodiments, the payload construct comprises a sequence of a payload flanked by ITR sequences. In some embodiments, expression of the sequence of the payload is driven by a constitutive promoter. In some embodiments, the constitutive promoter and sequence of the payload are flanked by ITR sequences. In some embodiments, the sequence of the payload comprises a polynucleotide sequence coding for a gene. In some embodiments, the gene codes for a selectable marker or detectable marker. In some embodiments, the gene codes for a therapeutic polypeptide or transgene. In some embodiments, the sequence of the payload comprises a polynucleotide sequence coding for a therapeutic polynucleotide. In some embodiments, the therapeutic polynucleotide is a tRNA suppressor or a guide RNA. In some embodiments, the guide RNA is a polyribonucleotide capable of binding to a protein. In some embodiments, the protein is nuclease. In some embodiments, the protein is a Cas protein, an ADAR protein, or an ADAT protein. In some embodiments, the Cas protein is catalytically inactive Cas protein. In some embodiments, the payload construct is stably integrated into the genome of the cell. In some embodiments, a plurality of the payload construct are stably integrated into the genome of the cell. In some embodiments, the plurality of the payload constructs are separately stably integrated into the genome of the cell. In some embodiments, the payload construct further comprises a sequence coding for a selectable marker or detectable marker outside of the ITR sequences. In some embodiments, the selectable marker is a mammalian cell selection element. In some embodiments, the selectable marker is an auxotrophic selection element. In some embodiments, the auxotrophic selection element codes for an active protein. In some embodiments, the active protein is DHFR. In some embodiments, the auxotrophic selection coding sequence encodes an inactive protein that requires expression of a second auxotrophic selection coding sequence for activity. In some embodiments, the second auxotrophic selection coding sequence encodes for DHFR Z-Cter or DHFR Z-Nter. In some embodiments, the inactive protein comprises a DHFR Z-Nter or DHFR Z-Cter In some embodiments, the selectable marker is DHFR Z-Nter or DHFR Z-Cter. The polynucleotide construct of any one of claims 2-6, wherein the DHFR Z-Nter comprises a sequence having at least 70%, 80%, 90%, 95%, 99%, or 100% sequence identity to SEQ ID NO: 4. The polynucleotide construct of any one of claims 2-6, wherein the DHFR Z-Cter comprises a sequence having at least 70%, 80%, 90%, 95%, 99%, or 100% sequence identity to SEQ ID NO: 5. In some embodiments, the selectable marker is an antibiotic resistance protein. In some embodiments, the selectable marker outside of the ITR sequences is a split intein linked to an N-terminus of the antibiotic resistance protein or split intein linked to a C-terminus of the antibiotic resistance protein. In some embodiments, the selectable marker outside of the ITR sequences is a leucine zipper linked to an N-terminus of the antibiotic resistance protein or leucine zipper linked to a C-terminus of the antibiotic resistance protein. In some embodiments, the antibiotic resistance protein is for puromycin resistance or blasticidin resistance. In some embodiments, the payload construct is in a plasmid. In some embodiments, the payload construct is in a bacterial artificial chromosome or yeast artificial chromosome. In some embodiments, the payload construct is stably integrated into the genome of the cell. In some embodiments, the payload construct is a synthetic nucleic acid construct. In some embodiments, the cell is capable of producing an rAAV virion that encapsidates the sequence of the payload. In some embodiments, the cell is capable of producing an rAAV virion upon addition of at least one triggering agent.

In some embodiments, this cell is capable of producing an rAAV virion upon addition of at least one triggering agent. In some embodiments, the rAAV virion comprising the capsid protein and the payload nucleic acid sequence have an infectivity of no less than 50%, 60%, 70%, 80%, 90%, 95%, or 99% at an MOI of 1×10⁵ vg/target cell or less. In some embodiments, the rAAV virions have an increased infectivity compared rAAV virions produced by an otherwise comparable the population of cells capable of producing rAAV virions upon transient transfection at the same MOI. In some embodiments, the rAAV virions have at least 1%, 5%, 10%, 15%, 20%, 30%, 40%, or 50% greater infectivity compared rAAV virions produced by an otherwise comparable the population of cells capable of producing rAAV virions upon transient transfection at the same MOI. In some embodiments, the rAAV virions have at least 50%, 60%, 70%, 80%, 90%, 95%, 97%, 98%, 99%, or 100% infectivity as compared to AAV virions produced by a cell having wildtype AAV at the same MOI. In some embodiments, the rAAV virions have at least 50%, 60%, 70%, 80%, 90%, 95%, 97%, 98%, 99%, or 100% infectivity as compared to AAV virions at the same MOI. In some embodiments, the AAV virions are wildtype AAV virions produced by a cell having wildtype AAV. In some embodiments, the MOI is 1×10¹, 1×10², 2×10³, 5×10⁴, or 1×10⁵ vg/target cell. In some embodiments, the MOI is selected from a range of 1×10¹ to 1×10⁵ vg/target cell. In some embodiments, the cell is conditionally capable of producing rAAV virions having a F:E ratio of no less than 0.5, 0.6, 0.7, 0.8, 0.9, 0.95, 0.97, or 0.99. In some embodiments, the rAAV virions have a F:E ratio of no less than 0.5, 0.6, 0.7, 0.8, 0.9, 0.95, 0.97, or 0.99 prior to purification. In some embodiments, the cell is conditionally capable of producing rAAV virions having a encapsidation ratio of no less than 0.5, 0.6, 0.7, 0.8, 0.9, 0.95, 0.97, or 0.99. In some embodiments, the rAAV virions have a payload encapsidation ratio of no less than 0.5, 0.6, 0.7, 0.8, 0.9, 0.95, 0.97, or 0.99 prior to purification. In some embodiments, the rAAV virions have a concentration of greater than 1×10¹¹ or no less than 5×10¹¹, 1×10¹², 5×10¹², 1×10¹³ or 1×10¹⁴ viral genomes per milliliter prior to purification. In some embodiments, the cell is capable of producing rAAV virions comprising the payload nucleic acid sequence at a titer of greater than 1×10¹¹ or no less than 5×10¹¹, 1×10¹², 5×10¹², 1×10¹³ or 1×10¹⁴ viral genomes per milliliter. In some embodiments, the cell is capable of producing rAAV virions comprising the payload nucleic acid sequence at a concentration of greater than 1×10¹¹ or no less than 5×10¹¹, 1×10¹², 5×10¹², 1×10¹³ or 1×10¹⁴ viral genomes per milliliter prior to purification. In some embodiments, this cell is expanded to produce a population of cells. In some embodiments, the population of cells produces a stable cell line as described herein. In some embodiments, this cell is passaged at least three times. In some embodiments, this cell can be passaged up to 60 times. In some embodiments, this cell can be passage more than 60 times. In some embodiments, the cell maintains the ability to be conditionally induced after each passage.

### 6.6. Population of Cells Comprising a Construct

In some embodiments, a population of cells comprise one construct (Rep/Cap construct, inducible helper construct, and the payload construct). In some embodiments, the one construct is stably integrated into the genomes of the cells. In some embodiments, a plurality of the one construct is stably integrated into the genomes of the cells. In some embodiments, a population of cells comprises two constructs (any combination of Rep/Cap construct, inducible helper construct, and the payload construct). In some embodiments, the two constructs are stably integrated into the genomes of the cells. In some embodiments, the two constructs are separately stably integrated into the genomes of the cells. In some embodiments, a plurality of the two constructs are stably integrated into the genome of the cell. In some embodiments, a plurality of the two constructs are separately stably integrated into the genomes of the cells. In some embodiments, a cell comprises the Rep/Cap construct and the inducible helper construct. In some embodiments, the cell, the inducible helper construct comprises a VA RNA construct as described herein. In some embodiments, cell further comprises the VA RNA construct as described herein. In some embodiments, the VA RNA construct is stably integrated into the genomes of the cells.

In some embodiments, a population of cells comprises all three constructs (Rep/Cap construct, inducible helper construct, and the payload construct). In some embodiments, the three constructs are stably integrated into the genomes of the cells. In some embodiments, the three constructs are separately stably integrated into the genomes of the cells. In some embodiments, a plurality of the three constructs are stably integrated into the genomes of the cells. In some embodiments, a plurality of the three constructs are separately stably integrated into the genomes of the cells. In some embodiments, the inducible helper construct comprises a VA RNA construct as described herein. In some embodiments, a population of cells further comprises the VA RNA construct separately integrated into the genomes of the cells.

A population of cells capable of producing rAAV virions having a encapsidation ratio of no less than 0.5, 0.6, 0.7, 0.8, 0.9, 0.95, 0.97, or 0.99. In some embodiments, the population of cells are a population of mammalian cells or a population of insect cells. In some embodiments, the population of cells are a population of HEK293 cells, HeLa cells, CHO cells, or SF9 cells. In some embodiments, the cell expresses E1A protein and E1B protein. In some embodiments, the population of cells further comprises a payload construct. In some embodiments, the payload construct comprises a sequence of a payload flanked by ITR sequences. In some embodiments, expression of the payload is driven by a constitutive promoter. In some embodiments, the constitutive promoter and sequence of the payload are flanked by ITR sequences. In some embodiments, the payload comprises a polynucleotide sequence encoding a gene. In some embodiments, the gene codes for a selectable marker or detectable marker. In some embodiments, the gene codes for a therapeutic polypeptide or transgene. In some embodiments, the payload comprises a polynucleotide sequence coding for a therapeutic polynucleotide. In some embodiments, the therapeutic polynucleotide is a tRNA suppressor or a guide RNA. In some embodiments, the guide RNA is a polyribonucleotide capable of binding to a protein. In some embodiments, the protein is nuclease. In some embodiments, the protein is a Cas protein, an ADAR protein, or an ADAT protein. In some embodiments, the Cas protein is catalytically inactive Cas protein. In some embodiments, the payload construct is stably integrated into the genome of the cell. In some embodiments, the payload construct further comprises a sequence coding for a selectable marker or detectable marker outside of the ITR sequences. In some embodiments, the selectable marker is an antibiotic resistance protein. In some embodiments, the selectable marker outside of the ITR sequences is a split intein linked to an N-terminus of the antibiotic resistance protein or split intein linked to a C-terminus of the antibiotic resistance protein. In some embodiments, the selectable marker outside of the ITR sequences is a leucine zipper linked to an N-terminus of the antibiotic resistance protein or leucine zipper linked to a C-terminus of the antibiotic resistance protein. In some embodiments, the antibiotic resistance protein is for puromycin resistance or blasticidin resistance. In some embodiments, the payload construct is in a plasmid. In some embodiments, the payload construct is in a bacterial artificial chromosome or yeast artificial chromosome. In some embodiments, the payload construct is stably integrated into the genomes of the population of cells. A population of cells produced by expanding a cell of any one of claims X. In some embodiments, expanding comprises passaging the cell at least three times. In some embodiments, a cell of the population of cells is capable of conditionally producing recombinant AAV (rAAV) virions upon addition of at least two triggering agents. In some embodiments, the cell is capable of conditionally producing rAAV virions upon addition of at least two triggering agents. In some embodiments, the at least two triggering agents comprise doxycycline and tamoxifen. In some embodiments, the at least two triggering agents induce the expression and translocation of an excising element to the nucleus. In some embodiments, a cell of the population of cells is capable of conditionally producing rAAV virions upon addition of an excising element. In some embodiments, the excising element is a recombinase. In some embodiments, the excising element is a site-specific recombinase. In some embodiments, the excising element is a Cre polypeptide or a flippase polypeptide. In some embodiments, the excising element is hormone regulated. In some embodiments, the population of cells are conditionally capable of producing rAAV virions within which are packaged an expressible polynucleotide encoding a payload; and wherein a population of virions produced by the population of cells are more homogenous than a population of virions produced by an otherwise comparable the population of cells capable of producing rAAV virions upon transient transfection. In some embodiments, the population of virions produced by the population of cells has a ratio of viral genomes to transduction units of about 500:1 to 1:1. In some embodiments, the population of virions produced by the population of cells has a ratio of vector genomes to infectious unit of 100:1. In some embodiments, production of virions is inducible upon addition of a triggering agent. In some embodiments, production of virions is inducible upon addition of at least two triggering agents. In some embodiments, the population of cells is conditionally capable of producing rAAV virions having a payload encapsidation ratio of no less than 0.5, 0.6, 0.7, 0.8, 0.9, 0.95, 0.97, or 0.99. In some embodiments, the rAAV virions have a payload encapsidation ratio of no less than 0.5, 0.6, 0.7, 0.8, 0.9, 0.95, 0.97, or 0.99 prior to purification. In some embodiments, the population of cells are capable of reaching a viable cell density of no less than 1×10⁶, 2×10⁶, 5×10⁶, or 1×10⁷ cells per milliliter. In some embodiments, the rAAV virions have a concentration of greater than 1×10¹¹ or no less than 5×10¹¹, 1×10¹², 5×10¹², 1×10¹³ or 1×10¹⁴ viral genomes per milliliter prior to purification. In some embodiments, the population of cells is capable of producing rAAV virions comprising the payload nucleic acid sequence at a titer of greater than 1×10¹¹ or no less than 5×10¹¹, 1×10¹², 5×10¹², 1×10¹³ or 1×10¹⁴ viral genomes per milliliter. In some embodiments, the population of cells is capable of producing rAAV virions comprising the payload nucleic acid sequence at a concentration of greater than 1×10¹¹ or no less than 5×10¹¹, 1×10¹², 5×10¹², 1×10¹³ or 1×10¹⁴ viral genomes per milliliter prior to purification. In some embodiments, the rAAV virions comprising the capsid protein and the payload nucleic acid sequence have an infectivity of no less than 50%, 60%, 70%, 80%, 90%, 95%, or 99% at an MOI of 1×10⁵ vg/target cell or less. In some embodiments, the rAAV virions have an increased infectivity compared rAAV virions produced by an otherwise comparable the population of cells capable of producing rAAV virions upon transient transfection at the same MOI. In some embodiments, the rAAV virions have at least 1%, 5%, 10%, 15%, 20%, 30%, 40%, or 50% greater infectivity compared rAAV virions produced by an otherwise comparable the population of cells capable of producing rAAV virions upon transient transfection at the same MOI. In some embodiments, the rAAV virions have at least 50%, 60%, 70%, 80%, 90%, 95%, 97%, 98%, 99%, or 100% infectivity as compared AAV virions AAV at the same MOI. In some embodiments, the AAV virions are wildtype AAV virions produced by a cell having wildtype AAV. In some embodiments, the MOI is 1×10¹, 1×10², 2×10³, 5×10⁴, or 1×10⁵ vg/target cell. In some embodiments, the MOI is selected from a range of 1×10¹ to 1×10⁵ vg/target cell. In some embodiments, the cell is conditionally capable of producing rAAV virions having a F:E ratio of no less than 0.5, 0.6, 0.7, 0.8, 0.9, 0.95, 0.97, or 0.99. In some embodiments, the rAAV virions have a F:E ratio of no less than 0.5, 0.6, 0.7, 0.8, 0.9, 0.95, 0.97, or 0.99 prior to purification. In some embodiments, the cells are cryopreserved. In some embodiments, the cells are comprised within a vial, flask, syringe, or other suitable cell-storage container. In some embodiments, production of rAAV virions is inducible in the absence of a plasmid. In some embodiments, expression of AAV Rep and Cap proteins is inducible in the absence of a plasmid. In some embodiments, expression of the at least one or more helper proteins is inducible in the absence of a plasmid. In some embodiments, production of rAAV virions is inducible in the absence of a transfection agent. In some embodiments, expression of AAV Rep and Cap proteins is inducible in the absence of a transfection agent. In some embodiments, expression of the at least one or more helper proteins is inducible in the absence of a transfection agent. A second population of cell produced by expanding the population of cells of any one of the preceding embodiments. The second population of cells, wherein expanding the population of cells comprises passaging the population of cells at least three times. In some embodiments expanding the population of cells comprises passaging the population of cells from 3 to 60 times. In some embodiments, expanding the population of cells comprises passaging the population of cells at least 4, 5, 6, 7, 8, 9, 10, 15, 20, 25, 30, 35, 40, 45, 50, 55, or 60 times.

In some embodiments, an rAAV virion produced by the methods described herein have increased infectivity compared to a comparable rAAV virion produced by transient transfection methods.

### 6.7. Stable Cell Line

In some embodiments, a stable cell line is produced from the cell as described herein. In some embodiments, a stable cell line is produced from the population of cells as described herein. In some embodiments, the stable cell line is derived from a single cell and is monclonal. The stable cell line can be a mammalian stable cell line. The stable cell line can be produced by expanding or passaging a cell as described herein.

In some embodiments, a stable cell line comprises the population of cells as disclosed herein. In some embodiments, the population of cells are derived from a single cell. In some embodiments, at least 70%, 80%, 90%, 95%, 99%, or 100% of the cells of the stable cell line are the population of cells as disclosed herein. A stable cell line derived from a cell as disclosed herein. A stable cell line expanded from a cell as disclosed herein. In some embodiments, the stable cell line is a mammalian stable cell line. In some embodiments, expression of one or more helper proteins is inducible in the absence of a plasmid. In some embodiments, expression of one or more helper proteins is inducible in the absence of a transfection agent. In some embodiments, expression of AAV Rep and Cap proteins is inducible in the absence of a plasmid. In some embodiments, expression of AAV Rep and Cap proteins is inducible in the absence of a transfection agent. In some embodiments, production of rAAV virions is inducible in the absence of a plasmid. In some embodiments, production of rAAV virions is inducible in the absence of a transfection agent. In some embodiments, the stable cell line is capable of producing rAAV virions comprising the payload nucleic acid sequence at a concentration of greater than 1×10¹¹ or no less than 5×10¹¹, 1×10¹², 5×10¹², 1×10¹³ or 1×10¹⁴ viral genomes per milliliter. In some embodiments, the stable cell line is capable of producing rAAV virions comprising the payload nucleic acid sequence at a concentration of greater than 1×10¹¹ or no less than 5×10¹¹, 1×10¹², 5×10¹², 1×10¹³ or 1×10¹⁴ viral genomes per milliliter prior to purification. In some embodiments, the stable cell line is conditionally capable of producing rAAV virions having a encapsidation ratio of no less than 0.5, 0.6, 0.7, 0.8, 0.9, 0.95, 0.97, or 0.99. In some embodiments, the rAAV virions have a encapsidation ratio of no less than 0.5, 0.6, 0.7, 0.8, 0.9, 0.95, 0.97, or 0.99 prior to purification. In some embodiments, the rAAV virions comprising the capsid protein and the payload nucleic acid sequence have an infectivity of no less than 50%, 60%, 70%, 80%, 90%, 95%, or 99%, at an MOI of 1×10⁵ vg/target cell or less. In some embodiments, the rAAV virions have an increased infectivity compared rAAV virions produced by an otherwise comparable the population of cells capable of producing rAAV virions upon transient transfection at the same MOI. In some embodiments, the rAAV virions have at least 1%, 5%, 10%, 15%, 20%, 30%, 40%, or 50% greater infectivity compared rAAV virions produced by an otherwise comparable the population of cells capable of producing rAAV virions upon transient transfection at the same MOI. In some embodiments, the rAAV virions have at least 50%, 60%, 70%, 80%, 90%, 95%, 97%, 98%, 99%, or 100% infectivity as compared to AAV virions produced by a cell having wildtype AAV at the same MOI. In some embodiments, the rAAV virions have at least 50%, 60%, 70%, 80%, 90%, 95%, 97%, 98%, 99%, or 100% infectivity as compared to AAV virions at the same MOI. In some embodiments, the AAV virions are wildtype AAV virions produced by a cell having wildtype AAV. In some embodiments, the MOI is 1×10¹, 1×10², 2×10³, 5×10⁴, or 1×10⁵ vg/target cell. In some embodiments, the MOI is selected from a range of 1×10¹ to 1×10⁵ vg/target cell. In some embodiments, the stable cell line is conditionally capable of producing rAAV virions having a F:E ratio of no less than 0.5, 0.6, 0.7, 0.8, 0.9, 0.95, 0.97, or 0.99. In some embodiments, the rAAV virions have a F:E ratio of no less than 0.5, 0.6, 0.7, 0.8, 0.9, 0.95, 0.97, or 0.99 prior to purification. In some embodiments, at least one cell of the stable cell line is cryopreserved. In some embodiments, at least one cell of the stable cell line is in a vial, flask, syringe, or other suitable cell-storage container.

In some embodiments, a method of producing a stable cell line comprises contacting a cell to the Rep/Cap construct as described herein, and expanding the cell to produce the stable cell line. In some embodiments, a method of producing a stable cell line comprises contacting a cell to the inducible helper construct as described herein, and expanding the cell to produce the stable cell line. In some embodiments, a method of producing a stable cell line comprises contacting a cell to the Rep/Cap construct, contacting the cell to the inducible helper construct as described herein, and expanding the cell to produce the stable cell line. In some embodiments, a method of producing a stable cell line comprises contacting a cell to the Rep/Cap construct, contacting the cell to inducible helper construct as described herein, contacting the cell to the payload construct, and expanding the cell to produce the stable cell line.

### 6.8. Cell Cultures and Bioreactors

In some embodiments, a cell, population of cells, or stable cell line as disclosed herein is in a cell culture. In some embodiments, a cell culture composition comprising: a) suspension-adapted cells, b)serum-free cell culture media, and c) recombinant AAV (rAAV) virions, wherein the cell culture composition is free of herpes simplex virus, baculovirus, and adenovirus, and wherein the cell culture composition is free of plasmid and transfection agent. In some embodiments, the cell culture composition is free of polyethylenimine (PEI). In some embodiments, the suspension-adapted cells are suspension-adapted mammalian cells. In some embodiments, the suspension-adapted cells are suspension-adapted HEK293 cells or derivatives thereof. In some embodiments, the suspension-adapted mammalian cells are cells from the stable cell line of as disclosed herein, the population of cells as disclosed herein, or comprise a cell as disclosed herein. In some embodiments, the the cell culture composition has a prepurification rAAV concentration of no less than 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, or 5×10¹⁵ viral genome (vg)/L. In some embodiments, the the cell culture composition has a prepurification rAAV encapsidation ratio of no less than 0.5, 0.6, 0.7, 0.8, 0.9, 0.95, 0.97, or 0.99.

In some embodiments, rAAV virion from the stable cells as disclosed herein is produced in a bioreactor. In some embodiments, a bioreactor comprises the stable cell line as disclosed herein. In some embodiments, a bioreactor comprising the population of cells of as disclosed herein. In some embodiments, a bioreactor comprising the cell as disclosed herein. In some embodiments, a bioreactor contains the cell culture as disclosed herein. In some embodiments, the bioreactor is a 1 L bioreactor. In some embodiments, the 1 L bioreactor has a total rAAV yield of greater than 1×10¹⁴ viral genome (vg). In some embodiments, the bioreactor is a 5 L bioreactor. In some embodiments, the 5 L bioreactor has a total rAAV yield of greater than 5×10¹⁴ viral genome (vg). In some embodiments, the bioreactor is a 50 L bioreactor. In some embodiments, the 50 L bioreactor has a total rAAV yield of greater than 5×10¹⁵ viral genome (vg). In some embodiments, the bioreactor is a 100 L bioreactor. In some embodiments, the 100 L bioreactor has a total rAAV yield of greater than 1×10¹⁶ viral genome (vg). In some embodiments, the bioreactor is a 500 L bioreactor. In some embodiments, the 500 L bioreactor has a total rAAV yield of greater than 5×10¹⁶ viral genome (vg). In some embodiments, the bioreactor is a 2000 L bioreactor. In some embodiments, the 2000 L bioreactor has a total rAAV yield of greater than 2×10¹⁷ viral genome (vg). In some embodiments, a bioreactor comprises a plurality of rAAV virions having a concentration of greater than 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, or 5×10¹⁵ viral genome (vg)/L. In some embodiments, a bioreactor comprises a plurality of rAAV virions having a prepurification concentration of greater than 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, or 5×10¹⁵ viral genome (vg)/L. In some embodiments, the bioreactor is a 1 L, 5 L, 50 L, 100 L, 500 L, or 2000 L bioreactor. In some embodiments, the bioreactor is a single use bioreactor.

### 6.9. Compositions of rAAV

In some embodiments, the cell, population of cells, or stable cell line as disclosed herein is induced (as disclosed herein, e.g., after administration of a first and a second triggering agent in a bioreactor) to produce a plurality of rAAV virons. In some embodiments, a composition comprises a plurality of rAAV virions encapsidating a viral genome, wherein the composition has a prepurification concentration of greater than 1×10¹¹ or no less than 5×10¹¹, 1×10¹², 5×10¹², 1×10¹³ or 1×10¹⁴ viral genomes per milliliter. In some embodiments, a composition comprises a plurality of rAAV virions encapsidating a viral genome, wherein the composition has a prepurification encapsidation ratio of no less than 0.5, 0.6, 0.7, 0.8, 0.9, 0.95, 0.97, or 0.99. In some embodiments, a composition comprises a plurality of rAAV virions encapsidating a viral genome, wherein the composition has a prepurification F:E ratio of no less than 0.5, 0.6, 0.7, 0.8, 0.9, 0.95, 0.97, or 0.99. In some embodiments, a composition comprises an rAAV virion encapsidating a viral genome, wherein the composition has an infectivity of no less than 50%, 60%, 70%, 80%, 90%, 95%, or 99% at an MOI of 1×10⁵ vg/target cell or less. In some embodiments, the rAAV virion has an increased infectivity compared an rAAV virion produced by an otherwise comparable cell capable of producing rAAV virions upon transient transfection at the same MOI. In some embodiments, the rAAV virion has at least 1%, 5%, 10%, 15%, 20%, 30%, 40%, or 50% greater infectivity compared an rAAV virion produced by an otherwise comparable cell capable of producing rAAV virions upon transient transfection at the same MOI. In some embodiments, the rAAV virion has at least 50%, 60%, 70%, 80%, 90%, 95%, 97%, 98%, 99%, or 100% infectivity as compared an AAV virion produced by a cell having wildtype AAV at the same MOI. In some embodiments, the rAAV virion has at least 50%, 60%, 70%, 80%, 90%, 95%, 97%, 98%, 99%, or 100% infectivity as compared an AAV virion produced by a cell having wildtype AAV at the same MOI. In some embodiments, the compositions further comprises a plurality of the rAAV virion. In some embodiments, the plurality of rAAV virions have a prepurification concentration of greater than 1×10¹¹ or no less than 5×10¹¹, 1×10¹², 5×10¹², 1×10¹³ or 1×10¹⁴ viral genomes per milliliter. In some embodiments, the plurality of rAAV virions have a prepurification encapsidation ratio of no less than 0.5, 0.6, 0.7, 0.8, 0.9, 0.95, 0.97, or 0.99. In some embodiments, the plurality of rAAV virions have a prepurification F:E ratio of no less than 0.5, 0.6, 0.7, 0.8, 0.9, 0.95, 0.97, or 0.99. In some embodiments, the plurality of rAAV virions have an infectivity of no less than 50%, 60%, 70%, 80%, 90%, 95%, or 99%. In some embodiments, the plurality of rAAV virions have an increased infectivity compared a plurality of rAAV virions produced by an otherwise comparable the population of cells capable of producing rAAV virions upon transient transfection at the same MOI. In some embodiments, the plurality of rAAV virions have at least 1%, 5%, 10%, 15%, 20%, 30%, 40%, or 50% greater infectivity compared a plurality of rAAV virions produced by an otherwise comparable the population of cells capable of producing rAAV virions upon transient transfection at the same MOI. In some embodiments, the plurality of rAAV virions have at least 50%, 60%, 70%, 80%, 90%, 95%, 97%, 98%, 99%, or 100% infectivity as compared a plurality of AAV virions produced by a cell having wildtype AAV at the same MOI. In some embodiments, the MOI is 1×10¹, 1×10², 2×10³, 5×10⁴, or 1×10⁵ vg/target cell. In some embodiments, the MOI is selected from a range of 1×10¹ to 1×10⁵ vg/target cell. In some embodiments, the viral genome comprises a sequence coding for a payload. In some embodiments, expression of the sequence of the payload is driven by a constitutive promoter. In some embodiments, the sequence of the payload comprises a polynucleotide sequence coding for a gene. In some embodiments, the gene codes for a selectable marker or detectable marker. In some embodiments, the gene codes for a therapeutic polypeptide or transgene. In some embodiments, the sequence of the payload comprises a polynucleotide sequence coding for a therapeutic polynucleotide. In some embodiments, the therapeutic polynucleotide is a tRNA suppressor or a guide RNA. In some embodiments, the guide RNA is a polyribonucleotide capable of binding to a protein. In some embodiments, the protein is nuclease. In some embodiments, the protein is a Cas protein, an ADAR protein, or an ADAT protein. In some embodiments, the Cas protein is catalytically inactive Cas protein. In some embodiments, the rAAV virion comprises a Cap polypeptide. In some embodiments, the Cap polypeptide is an AAV capsid protein. In some embodiments, the AAV capsid protein is VP1, VP2, or VP3. In some embodiments, a serotype of the AAV capsid protein is selected from the group consisting of AAV1, AAV2, AAV3, AAV4, AAV5, AAV6, AAV7, AAV8, AAV9, AAV 10, AAV11, AAV 12, AAV13, AAV 14, AAV 15 and AAV 16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, and AAVhu68.

In some embodiments, rAAV virions as disclosed herein are in a first composition and a second composition. In some embodiments, the first composition and the second composition have an encapsidation ratio that varies by no more than 20%, 10%, 5%, 4%, 3%, 2%, or 1%. In some embodiments, the first composition and the second composition have an F:E ratio that varies by no more than 20%, 10%, 5%, 4%, 3%, 2%, or 1%. In some embodiments, the first composition and the second composition have a concentration of viral genomes per milliliter that varies by no more than 20%, 10%, 5%, 4%, 3%, 2%, or 1%. In some embodiments, the first composition and the second composition have an infectivity that varies by no more than 20%, 10%, 5%, 4%, 3%, 2%, or 1%. In some embodiments, the first composition is a first dose and the second composition is a second dose. In some embodiments, the first composition is produced at least 1, 2, 3, 4, 5, 6, or 7 days before the second composition is produced. In some embodiments, a plurality of rAAV virions of the first composition is produced at least 1, 2, 3, 4, 5, 6, or 7 days before a plurality of rAAV virions of the second composition is produced. In some embodiments, the first composition is produced at least 1, 2, 3, 4, 5, 6, 7, 8, 9, 10, 11, or 12 months before the second composition is produced. In some embodiments, a plurality of rAAV virions of the first composition is produced at least 1, 2, 3, 4, 5, 6, 7, 8, 9, 10, 11, or 12 months before the second composition is produced. In some embodiments, the first composition is produced at least 1, 2, 3, 4, 5, 6, 7, 8, 9, 10, 11, or 12 years before the second composition is produced. In some embodiments, a plurality of rAAV virions of the first composition is produced at least 1, 2, 3, 4, 5, 6, 7, 8, 9, 10, 11, or 12 years before the second composition is produced. In some embodiments, the first composition is produced from a plurality of virions from a first bioreactor and the second composition is produced from a plurality of virions from a second bioreactor. In some embodiments, a third composition or more compositions are produced from the rAAV as disclosed herein. In some embodiments, the first composition, the second composition, and the third composition have an encapsidation ratio that varies by no more than 20%, 10%, 5%, 4%, 3%, 2%, or 1%. In some embodiments, the first composition, the second composition, and the third composition have an F:E ratio that varies by no more than 20%, 10%, 5%, 4%, 3%, 2%, or 1%. In some embodiments, the first composition, the second composition, and the third composition have a concentration of viral genomes per milliliter that varies by no more than 20%, 10%, 5%, 4%, 3%, 2%, or 1%. In some embodiments, the first composition, the second composition, and the third composition have an infectivity that varies by no more than 20%, 10%, 5%, 4%, 3%, 2%, or 1%. In some embodiments, the third composition is a third dose. In some embodiments, the third composition is produced at least 1, 2, 3, 4, 5, 6, or 7 days after the second composition is produced. In some embodiments, a plurality of rAAV virions of the third composition is produced at least 1, 2, 3, 4, 5, 6, or 7 days after a plurality of rAAV virions of the second composition is produced. In some embodiments, the third composition is produced at least 1, 2, 3, 4, 5, 6, 7, 8, 9, 10, 11, or 12 months after the second composition is produced. In some embodiments, a plurality of rAAV virions of the third composition is produced at least 1, 2, 3, 4, 5, 6, 7, 8, 9, 10, 11, or 12 months after the second composition is produced. In some embodiments, the third composition is produced at least 1, 2, 3, 4, 5, 6, 7, 8, 9, 10, 11, or 12 years after the second composition is produced. In some embodiments, a plurality of rAAV virions of the third composition is produced at least 1, 2, 3, 4, 5, 6, 7, 8, 9, 10, 11, or 12 years after the second composition is produced. In some embodiments, the third composition is produced from a plurality of virions from a third bioreactor.

### 6.10. Pharmaceutical Compositions

In some embodiments, a pharmaceutical composition comprises the plurality of rAAV virions of claims as disclosed herein and a pharmaceutically acceptable carrier. In some embodiment, a plurality of pharmaceutical doses each independently comprise the plurality of rAAV virions of claims as disclosed herein and a pharmaceutically acceptable carrier. In some embodiments, the encapsidation ratio has a difference of not more than 20%, 10%, 5%, 4%, 3%, 2%, or 1% between a first dose and a second dose of a plurality of pharmaceutical doses. In some embodiments, the F:E ratio has a difference of not more than 20%, 10%, 5%, 4%, 3%, 2%, or 1% between a first dose and a second dose of a plurality of pharmaceutical doses. In some embodiments, the concentration of viral genomes has a difference of not more than 20%, 10%, 5%, 4%, 3%, 2%, or 1% between a first dose and a second dose of a plurality of pharmaceutical doses. In some embodiments, the concentration of vector genomes has a difference of not more than 20%, 10%, 5%, 4%, 3%, 2%, or 1% between a first dose and a second dose of a plurality of pharmaceutical doses. In some embodiments, the rAAV virion infectivity has a difference of not more than 20%, 10%, 5%, 4%, 3%, 2%, or 1% between a first dose and a second dose of a plurality of pharmaceutical doses.

### 6.11. Method of Producing rAAV

In another aspect, methods of producing rAAV from stable cell lines is provided. The method comprises adding the at least first and at least second expression triggering agents to the medium within which the stable mammalian cell lines described above are being cultured.

In particular embodiments, the first expression triggering agent is a tetracycline. In specific embodiments, the first expression triggering agent is Dox. In particular embodiments, the second expression triggering agent is an estrogen agonist or selective estrogen receptor modulator. In specific embodiments, the second expression triggering agent is tamoxifen.

In some embodiments, the method further comprises a later step of culturing the stable mammalian cell line only in the presence of the first expression triggering agent.

In some embodiments, the method further comprises purifying rAAV from culture medium. In some embodiments, the purifying comprises performing chromatographic purification. In some embodiments, the chromatographic purification comprises using a positively charged anion exchange resin, using a negatively charged anion exchange resin, using cation exchange chromatography, using affinity chromatography, using size exclusion chromatography, or a combination thereof. In some embodiments, the chromatographic purification comprises using column chromatographic fractionation.

In some embodiments, rAAV is produced in a bioreactor as described herein.

In some embodiments, a method of inducing the cell as described herein, the population of cells as described herein, or the stable cell line as described herein comprises administering a first triggering agent to the cell, population of cells, or the stable cell line, thereby inducing expression of the Rep polypeptides, Cap polypeptides, and one or more adenoviral helper proteins, in the cell, population of cells, or stable cell line. In some embodiments, the first triggering agent binds to an activator or a repressor. In some embodiments, activation of an inducible promoter is induced. In some embodiments, the activated inducible promoter transcribes a recombinase. In some embodiments, the first triggering agent is tetracycline or cumate. In some embodiments, the tetracycline is doxycycline. The methods described herein further comprise culturing the cell, population of cells, or the stable cell line with a second triggering agent. In some embodiments, the second triggering agent is an estrogen receptor ligand. In some embodiments, the second triggering agent is a selective estrogen receptor modulator (SERM). In some embodiments, the second triggering agent is tamoxifen. In some embodiments, the second triggering agent binds to the recombinase. In some embodiments, the second triggering agent induces the recombinase to translocate to a nucleus of the cell, of a cell of the population of cells, of a cell of the stable cell lines.

In some embodiments, a method of producing rAAV virion comprises administering a first triggering agent to the cell, population of cells, or the stable cell line, administering a second triggering agent to the cell, population of cells, or stable cell line, thereby producing the rAAV virion in the cell, population of cells, or stable cell line. In some embodiments, the first triggering agent binds to an activator or a repressor. In some embodiments, activation of an inducible promoter is induced. In some embodiments, the activated inducible promoter transcribes a recombinase. In some embodiments, the first triggering agent is tetracycline or cumate. In some embodiments, the tetracycline is doxycycline. The method of any one of claims X, further comprising culturing the cell, population of cells, or the stable cell line with a second triggering agent. In some embodiments, the second triggering agent is an estrogen receptor ligand. In some embodiments, the second triggering agent is a selective estrogen receptor modulator (SERM). In some embodiments, the second triggering agent is tamoxifen. In some embodiments, the second triggering agent binds to the recombinase. In some embodiments, the second triggering agent induces the recombinase to translocate to a nucleus of the cell, of a cell of the population of cells, of a cell of the stable cell lines. In some embodiments, the recombinase cuts at recombinase sites. In some embodiments, the at least one adenoviral help proteins, the Rep polypeptides, and the Cap polypeptides are expressed. In some embodiments, the Rep polypeptides and the Cap polypeptides assemble into an rAAV virion. In some embodiments, the rAAV virion encapsidates a sequence of a payload. In some embodiments, the cell, population of cells, or stable cell line do not express cytotoxic levels of Rep polypeptides prior to administration of both the first triggering agent and the second triggering agent. In some embodiments, the cell, population of cells, or stable cell line do not express cytotoxic levels of Cap polypeptides prior to administration of both the first triggering agent and the second triggering agent. In some embodiments, the cell, population of cells, or stable cell line do not express cytostatic levels of Rep polypeptides prior to administration of both the first triggering agent and the second triggering agent. In some embodiments, the average concentration of Rep polypeptides within the cell, population of cells, or stable cell line is less than the amount prior to administration of both of the first triggering agent and second triggering agent. In some embodiments, expression of Rep polypeptides and Cap polypeptides becomes constitutive after administration of both the first triggering agent and the second triggering agent. The method of any one of claims X, further comprising performing at least a portion of the method in a bioreactor. In some embodiments, the bioreactor is not less than 20 L, 30, L, 40 L, 50 L, 100 L, 250 L, 300 L, or 500 L.

In some embodiments, the method further comprises producing the rAAV virions in a plurality of batches. In some embodiments, the method further comprises producing the rAAV virions having a difference in the encapsidation ratio of not more than 20%, 15%, 10%, 5%, 3%, 2%, or 1% between a first batch and a second batch. In some embodiments, the method further comprises producing the rAAV virions having a difference in the F:E ratio of not more than 20%, 15%, 10%, 5%, 3%, 2%, or 1% between a first batch and a second batch. In some embodiments, the method further comprises producing the rAAV virions having a difference in the concentration of viral genomes of not more than 20%, 15%, 10%, 5%, 3%, 2%, or 1% between a first batch and a second batch. In some embodiments, the method further comprises producing the rAAV virions having a difference in the concentration of vector genomes of not more than 20%, 15%, 10%, 5%, 3%, 2%, or 1% between a first batch and a second batch. In some embodiments, the method further comprises producing the rAAV virions having a difference in infectivity of not more than 20%, 15%, 10%, 5%, 3%, 2%, or 1% between a first batch and a second batch. In some embodiments, the method further comprises performing the method according to good manufacturing practice (GMP) standards. In some embodiments, the method further comprises performing the method in a GMP facility. In some embodiments, the method further comprises comprising culturing the cells in a culture medium and collecting a portion of the plurality of rAAV virions from the culture medium. In some embodiments, the method further comprises purifying at least some of the plurality of rAAV virions collected from the culture medium to obtain a purified rAAV population. In some embodiments, the purifying comprises performing chromatographic purification. In some embodiments, the chromatographic purification comprises using a positively charged anion exchange resin, using a negatively charged anion exchange resin, using cation exchange chromatography, using affinity chromatography, using size exclusion chromatography, or a combination thereof. In some embodiments, the chromatographic purification comprises using column chromatographic fractionation.

In some embodiments, an rAAV virion is made by the methods as disclosed herein. In some embodiments, a composition comprising a plurality of rAAV virions is made by the methods as disclosed herein. In some embodiments, the rAAV virion produced as disclosed herein has increased infectivity compared to an rAAV virion produced by comparable transient transfection methods.

### 6.12. Methods of Treatment

In another aspect, methods of treatment are provided. In various embodiments, the method comprises administering rAAV produced by the process described above to a patient in need thereof. In some embodiments, the administering is by intravenous administration, intramuscular administration, intrathecal administration, intracisternal administration, or administration via brain surgery.

In some embodiments, a method of treating a condition or disorder comprises administering a therapeutically effective amount of the pharmaceutical composition of as disclosed herein to a patient in need thereof. In some embodiments, the disorder is a monogenic disorder. In some embodiments, the treatment results in at least one undesirable side effect and wherein the undesirable side effect is reduced relative to administering a daily dose that deviates more than 50%, 40%, 30%, 30%, 15%, 10%, 5%, or 2% from an expected dose. In some embodiments, the administering is by injection. In some embodiments, the injection is an infusion. In some embodiments, the daily dose is administered to the patient once. In some embodiments, the daily dose is administered to the patient two or more times. In some embodiments, the treatment results in at least one undesirable side effect and wherein the undesirable side effect is reduced relative to administering a plurality of rAAV virions produced from a triple transfection method.

In some embodiments, the methods reduce the immunogenicity of a dose of rAAV having a predetermined number of viral genomes (VG) as compared to the same rAAV VG dose prepared by transient triple transfection. In some embodiments, the immunogenicity is measured by the titer or concentration of neutralizing antibodies in a subject. In some embodiments, a concentration of rAAV virion neutralizing antibody in the blood serum of the patient is reduced relative to a concentration of rAAV virion neutralizing antibody in the blood serum of a patient after administering a plurality of rAAV virions produced from a triple transfection method. In some embodiments, the concentration of rAAV virion neutralizing antibodies is measured by an ELISA assay.

In some embodiments, the methods reduce the number or intensity of adverse effects caused by administering a dose of rAAV having a predetermined number of viral genomes (VG) as compared to the same rAAV VG dose prepared by transient triple transfection. In some embodiments, the methods reduce the number of adverse effects. In some embodiments, the predetermined number of VG in a dose is no greater than 3×10¹⁴ vg/kg. In some embodiments, the predetermined number of VG in a dose is no greater than 1×10¹⁴ vg/kg. In some embodiments, the predetermined number of VG in a dose is no greater than 5×10¹³ vg/kg. In some embodiments, the methods reduce the intensity of adverse effects. In some embodiments, the methods reduce both the number and the intensity of adverse events.

In some embodiments, a method of administering a dose of rAAV virions having a predetermined number of viral genomes (VG) to a subject with reduced number or intensity of adverse effects as compared to administration of the same rAAV VG dose prepared by transient triple transfection comprises: administering a dose of rAAV produced in the cell as disclosed herein, the population of cells disclosed herein, or the stable cells as disclosed herein. In some embodiments, the adverse effect is selected from the group consisting of: liver dysfunction, liver inflammation, gastrointestinal infection, vomiting, bacterial infection, sepsis, increases in troponin levels, decreases in red blood cell counts, decreases in platelet counts, activation of the complement immune system response, acute kidney injury, cardio-pulmonary insufficiency, and death. In some embodiments, the adverse effect is an increase in serum levels of one or more proinflammatory cytokines. In some embodiments, the adverse effect is an increase in serum levels of one or more of interferon gamma (IFNγ), interleukin 1β (IL-1β), and interleukin 6 (IL-6).

In another aspects, a method of repeatedly administering a dose of rAAV to a subject in need thereof are provided. In some embodiments, the method comprises administering a first dose of rAAV produced by the cell lines and the processes described above, and then administering at least a second dose of rAAV produced by the cell lines and the processes described above. In some embodiments, the method comprises administering a first dose and a second dose of rAAV produced by the cell lines and the processes described above. In some embodiments, the method comprises administering a first dose, a second dose, and a third dose of rAAV produced by the cell lines and the processes described above. In some embodiments, the method comprises administering more than three doses of rAAV produced by the cell lines and the processes described above. In some embodiments, the first dose of rAAV and the at lease second dose of rAAV are administered through the same route of administration. In some embodiments, the first dose of rAAV and the at least second dose of rAAV are administered through different routes of administration. In some embodiments, the route of administration is intravenous administration, intramuscular administration, intrathecal administration, intracisternal administration, or administration via brain surgery.

In some embodiments, a method of treating a condition or disorder comprises administering a first therapeutically effective amount of the pharmaceutical composition of as disclosed herein having a predetermined number of viral genomes to a patient in need thereof and a second therapeutically effective amount of the pharmaceutical composition as disclosed herein having the predetermined number of viral genomes to the patient in need thereof. In some embodiments, the first therapeutically effective amount and the second therapeutically effective amount vary by no more than 1%, 5%, 10%, or 15%.

### 6.13. Kits

In another aspect, components or embodiments described herein for the system are provided in a kit. For example, any of the plasmids, as well as the mammalian cells, related buffers, media, triggering agents, or other components related to cell culture and virion production can be provided, with optional components frozen and packaged as a kit, alone or along with separate containers of any of the other agents and optional instructions for use. In some embodiments, the kit may comprise culture vessels, vials, tubes, or the like.

The methods for producing and packaging recombinant vectors in desired AAV capsids to produce the rAAVs are not meant to be limiting and other suitable methods will be apparent to the skilled artisan.

### 6.14. Aspects of the Invention

The below items disclose various aspects of the invention. Each of the aspects described below can be combined with other aspects and embodiments disclosed elsewhere herein, including the claims, where the combinations are clearly compatible. For example, described herein are three exemplary constructs, referred to as “construct 1”, “construct 2” and “construct 3”. The disclosure provided herein describes these constructs in specific and general detail.

In the following aspects, the first recombinant nucleic acid sequence encoding an AAV Rep protein and an AAV Cap protein corresponds to the specific and general disclosures of “construct 1” provided herein. It is intended that any aspects described below relating to the first recombinant nucleic acid may be combined with any of the specific and general disclosures of “construct 1” provided herein where the combinations are clearly compatible.

In the following aspects, the second recombinant nucleic acid sequence encoding one or more adenoviral helper proteins corresponds to the specific and general disclosures of “construct 2” provided herein. It is intended that any aspects described below relating to the second recombinant nucleic acid may be combined with any of the specific and general disclosures of “construct 2” provided herein where the combinations are clearly compatible.

In the following aspects, the third recombinant nucleic acid sequence encoding a payload corresponds to the specific and general disclosures of “construct 3” provided herein. It is intended that any aspects described below relating to the third recombinant nucleic acid may be combined with any of the specific and general disclosures of “construct 3” provided herein where the combinations are clearly compatible.

It is intended that any aspects and disclosures provided herein relating to the first, second and third recombinant nucleic acids, and relating to the specific and general disclosures of constructs 1, 2 and 3 may be combined together where the combinations are clearly compatible.

1. A composition comprising one or more nucleic acids which together comprise:
(i) a first recombinant nucleic acid sequence encoding an AAV Rep protein and an AAV Cap protein; and
(ii) a second recombinant nucleic acid sequence encoding one or more adenoviral helper proteins,
wherein when the one or more nucleic acids are integrated into the nuclear genome of a mammalian cell the AAV Rep protein, the AAV Cap protein, and/or the one or more adenoviral helper proteins are conditionally expressible and thereby conditionally produce recombinant AAV (rAAV) virions.
   2. The composition of aspect 1, wherein the conditional expression of the AAV Rep protein, the AAV Cap protein, and/or the one or more adenoviral helper proteins is controlled by one or more excisable elements present in the one or more nucleic acids.
   3. The composition of aspect 2, wherein the one or more excisable elements comprise one or more introns and/or one or more exons.
   4. The composition of any one of the preceding aspects, wherein the first recombinant nucleic acid sequence encodes:
a) a first part of the AAV Rep protein coding sequence;
b) the second part of the AAV Rep protein coding sequence;
c) an excisable element between the first part of the AAV Rep protein coding sequence and the second part of the AAV Rep protein coding sequence; and
d) the AAV Cap protein coding sequence.
   5. The composition of any one of aspects 2-4, wherein the excisable element comprises:
a) a first spacer segment comprising a first intron,
b) a second spacer segment comprising a coding sequence of a detectable marker; and
c) a third spacer segment comprising a second intron, and wherein the first spacer segment and the third spacer segment are capable of being excised by endogenous cellular machinery of a mammalian cell.
   6. The composition of aspect 5, wherein the excisable element comprises from 5′ to 3′:
a) a 5′ splice site;
b) a first spacer segment comprising a first intron;
c) a second spacer segment comprising:
   i) a first lox sequence;
   ii) a 3′ splice site;
   iii) an exon;
   iv) a stop signaling sequence; and
   v) a second lox sequence; and
d) a third spacer segment comprising a second intron.
   7. The composition of aspect 5 or aspect 6, wherein the detectable marker is a luminescent marker, a radiolabel or a fluorescent marker, optionally a fluorescent marker which is GFP, EGFP, RFP, CFP, BFP, YFP, or mCherry.
   8. The composition of any one of aspects 5-7, wherein:
a) the first spacer segment comprises a nucleic acid sequence having at least 80% identity to SEQ ID NO: 1; and/or
b) the second spacer segment comprises a nucleic acid sequence having at least 80% identity to SEQ ID NO: 2; and/or
c) the third spacer segment comprises a nucleic acid sequence having at least 80% identity to SEQ ID NO: 3.
   9. The composition of any one of aspects 5-8, wherein the second spacer segment is capable of being excised by a Cre polypeptide.
   10. The composition of any one of the preceding aspects, wherein the expression of the AAV Rep protein and/or the AAV Cap protein is driven by native promoters.
   11. The composition of aspect 10, wherein:
a) the native promoters P5 and/or P19 drive the expression of the AAV Rep protein; and/or
b) the native promoter P40 drives the expression of the AAV Cap protein.
   12. The composition of any one of the preceding aspects, wherein the second recombinant nucleic acid sequence encodes:
a) one or more adenoviral helper proteins;
b) a conditionally self-excising element; and
c) an inducible promoter;
   wherein, once integrated into the nuclear genome of a mammalian cell, the expression of the one or more adenoviral helper protein coding sequences is under the control of the conditionally self-excising element and the inducible promoter.
   13. The composition of aspect 12, wherein the one or more adenoviral helper proteins comprise E2A and E4.
   14. The composition of aspect 12 or aspect 13, wherein the self-excising element comprises a sequence which encodes a polypeptide, preferably a recombinase polypeptide, more preferably a Cre polypeptide.
   15. The composition of aspect 14, wherein the polypeptide encoded by the self-excising element is conditionally expressible and is expressed only in the presence of a triggering agent.
   16. The composition of aspect 15, wherein the triggering agent is a hormone, preferably tamoxifen.
   17. The composition of any one of aspects 9-16, wherein the inducible promoter is a Tet inducible promoter.
   18. The composition of any one of aspects 12-17, wherein the second recombinant nucleic acid sequence further comprises a sequence that encodes a Tet responsive activator protein, preferably Tet-on-3G.
   19. The composition of aspect 18, wherein the expression of Tet-On 3G activator protein is driven by an Elalpha promoter.
   20. The composition of any one of aspects 12-19, wherein the second recombinant nucleic acid sequence comprises a sequence with at least 80% homology, at least 90% homology, at least 95% homology, at least 99% homology, or a sequence identical to SEQ ID NO: 11 or SEQ ID NO: 12.
   21. The composition of any one of the preceding aspects, wherein the one or more nucleic acids further comprises a nucleic acid sequence encoding a VA RNA sequence.
   22. The composition of aspect 21, wherein the expression of VA RNA is constitutive.
   23. The composition of aspect 21, wherein the expression of VA RNA is inducible.
   24. The composition of aspect 23, wherein the VA RNA sequence comprises one or more mutations in the VA RNA internal promoter, preferably G16A and G60A.
   25. The composition of any one of aspects 21 to 24, wherein the expression of VA RNA is driven by a Elalpha promoter or a U6 promoter.
   26. The composition of aspect 25, wherein the expression of VA RNA is driven by a U6 promoter, and wherein the U6 promoter comprises:
a) a first part of a U6 promoter sequence,
b) a stuffer sequence, and
c) a second part of a U6 promoter sequence, and
   wherein the stuffer sequence is capable of being excised by a Cre polypeptide.
   27. The composition of any one of the preceding aspects, wherein a serotype of the AAV Cap protein is selected from the group consisting of AAV1, AAV2, AAV3, AAV4, AAV5, AAV6, AAV7, AAV8, AAV9, AAV 10, AAV11, AAV 12, AAV13, AAV 14, AAV 15 and AAV 16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16 and AAVhu68.
   28. The composition of aspect 27, wherein the serotype is an AAV5 and the Cap protein that comprises one or more mutations or insertions.
   29. The composition of any one of the preceding aspects, wherein the one or more recombinant nucleic acids further encode a third recombinant nucleic acid sequence encoding a payload, optionally wherein the payload is:
(a) a polynucleotide payload, such as a guide RNA for RNA editing, a guide RNA for Cas protein-directed DNA editing, a tRNA suppressor, or a gene for replacement gene therapy; or
(b) a protein such as a therapeutic antibody or a vaccine immunogen.
   30. The composition of any one of the preceding aspects, wherein the one or more recombinant nucleic acids comprise one or more mammalian cell selection elements.
   31. The composition of aspect 30, wherein one or more of the mammalian cell selection elements encodes an antibiotic resistance gene, optionally a blasticidin resistance gene.
   32. The composition of aspect 30 or aspect 31, wherein one or more of the mammalian cell selection elements is an auxotrophic selection element which encodes an active protein, preferably wherein the protein is DHFR.
   33. The composition of aspect 30 or aspect 31, wherein one or more of the mammalian cell selection elements is a first auxotrophic selection element which encodes an inactive protein that requires expression of a second inactive protein from a second auxotrophic selection coding sequence for activity.
   34. The composition of aspect 33, wherein the first auxotrophic selection coding sequence encodes for DHFR Z-Cter (SEQ ID NO: 5) activity, and/or wherein the second auxotrophic selection coding sequence encodes for DHFR Z-Nter (SEQ ID NO: 4).
   35. The composition of any one of aspects 1-30, wherein:
a) the first recombinant nucleic acid comprises a mammalian cell selection element which encodes an antibiotic resistance gene, preferably a blasticidin resistance gene; and
b)
   i. the second recombinant nucleic acid comprises a first auxotrophic selection element which encodes an inactive protein that requires expression of a second inactive protein from a second auxotrophic selection coding sequence for activity; and
   ii. the third recombinant nucleic acid comprises the second auxotrophic selection element which encodes the inactive protein that requires expression of the first inactive protein from the first auxotrophic selection coding sequence for activity; and
wherein in (i) or (ii) the first auxotrophic selection coding sequence encodes for DHFR Z-Cter (SEQ ID NO: 5), and the second auxotrophic selection coding sequence encodes for DHFR Z-Nter (SEQ ID NO: 4) or wherein the first auxotrophic selection coding sequence encodes for DHFR Z-Nter (SEQ ID NO: 4), and the second auxotrophic selection coding sequence encodes for DHFR Z-Cter (SEQ ID NO: 5).
   36. A mammalian cell wherein the nuclear genome of the cell comprises a plurality of integrated recombinant nucleic acid constructs which together encode for a recombinant adeno-associated virus (rAAV) virions, wherein the rAAV virions can be conditionally expressed from the cell.
   37. The mammalian cell of aspect 36, wherein the plurality of integrated recombinant nucleic acid constructs comprise the one or more recombinant nucleic acids of any one of aspects 1-35, wherein the AAV Rep protein, the AAV Cap protein and/or the adenoviral helper proteins can be conditionally expressed from the cell.
   38. The mammalian cell of aspect 36 or aspect 37, wherein the cell line expresses adenoviral helper proteins E1A and E1B.
   39. A mammalian cell of any one of aspects 36-38, wherein the plurality of integrated recombinant nucleic acid constructs comprise:
(i) a first integrated polynucleotide construct comprising:
   a) a first part of an AAV Rep protein coding sequence;
   b) a second part of an AAV Rep protein coding sequence;
   c) an excisable element between the first part of the AAV Rep protein coding sequence and the second part of the AAV Rep protein coding sequence, wherein the excisable element comprises:
      i) a first spacer segment comprising a first intron;
      ii) a second spacer segment comprising a coding sequence of a detectable marker, wherein the second spacer segment is capable of being excised by a Cre polypeptide; and
      iii) a third spacer segment comprising a second intron; and
   d) an AAV Cap protein coding sequence;
   wherein the AAV Rep protein and the AAV Cap protein is driven by the native promoters P5, P19, and P40;
(ii) a second integrated polynucleotide construct comprising
   a) a conditionally expressible VA RNA coding sequence which comprises a mutation in the VA RNA internal promoter, wherein the expression of VA RNA is driven by a U6 promoter, optionally wherein the VA RNA sequence comprises G16A and G60A mutations;
   b) one or more adenoviral helper protein coding sequences, wherein the adenoviral helper proteins are E2A and E4;
   c) a conditionally self-excising element which encodes a Cre polypeptide which translocates to the nucleus and self-excises only in the presence of a triggering agent which is tamoxifen, and
   c) an inducible promoter which is a Tet inducible promoter, and
   wherein the expression of the one or more adenoviral helper protein coding sequences is under the control of the conditionally self-excising element and the inducible promoter; and
(iii) a third integrated polynucleotide construct comprising encodes for the payload, wherein the payload is a polynucleotide payload.
   40. A method of producing a population of rAAV virions comprising:
(a) culturing the cell of any one of aspects 36-39 in conditions which allow for the expression of the rAAV virions; and
(b) isolating the rAAV virions from the cell culture.
   41. The method of aspect 40, wherein the prepurification rAAV viral genome (VG) to viral particle (VG:VP) ratio of greater than 0.5.
   42. The method of aspect 40 or aspect 41, wherein the population of rAAV virions produced by the cell has:
(a) a ratio of viral genomes to transduction units of about 500 to 1 to 1 to 1; and/or
(b) a ratio of vector genomes to infectious unit of 100:1.
   43. A method of preparing the cell of any one of aspects 36-39 comprising:
i) providing a mammalian cell and the one or more nucleic acids of any one of aspects 1-35; and
ii) integrating the one or more nucleic acids of any one of aspects 1-35 into the nuclear genome of the mammalian cell.
   44. A population of rAAV virions produced by the method of any one of aspects 40-42.
   45. The population of rAAV virions of aspect 44, wherein the infectivity of the virions is at least 50% at an MOI of 10000.
   46. A pharmaceutical composition comprising a population of rAAV virions according to aspect 44 or aspect 45, for use as a medicament, optionally for use in treating a monogenic disorder.
   47. The population of rAAV virions according to aspect 44 or aspect 45 or the pharmaceutical composition according to aspect 46, for use as a medicament, optionally for use in treating a monogenic disorder.
   48. The population of rAAV virions or the pharmaceutical composition for use according to aspect 47, wherein the rAAV virions are administered at a dosage of 4×10¹⁴ or lower.

### 6.15. Numbered Embodiments #1

[1] A polynucleotide construct coding for:
   a) a first part of a Rep polypeptide;
   b) a second part of a Rep polypeptide;
   c) a Cap polypeptide; and
   d) an excisable element positioned between the first part of the Rep polypeptide and the second part of the Rep polypeptide.
[2] The polynucleotide construct of embodiment 1, wherein the Rep polypeptide is a wildtype Rep polypeptide.
[3] The polynucleotide construct of any one of embodiments 1-2, wherein the Cap polypeptide is a wildtype Cap polypeptide.
[4] The polynucleotide construct of any one of embodiments 1-3, wherein the excisable element comprises an intron.
[5] The polynucleotide construct of any one of embodiments 1-4, wherein the excisable element comprises an exon.
[6] The polynucleotide construct of any one of embodiments 1-5, wherein the excisable element comprises an intron and an exon.
[7] The polynucleotide construct of any one of embodiments 1-6, wherein the excisable element comprises from 5′ to 3′:
a) a 5′ splice site;
b) a first spacer segment comprising a first intron;
c) a second spacer segment comprising:
   i) a first lox sequence;
   ii) a 3′ splice site;
   iii) an exon;
   iv) a stop signaling sequence; and
   v) a second lox sequence; and
d) a third spacer segment comprising a second intron.
   wherein the first spacer segment and the third spacer segment are capable of being excised by endogenous cellular machinery.
[8] The polynucleotide construct of embodiment 7, wherein the first and second lox sequences are loxP sequences.
[9] The polynucleotide construct of embodiment 7, wherein the second spacer segment is excisable by a Cre polypeptide.
[10] The polynucleotide construct of embodiment 9, wherein the Cre polypeptide is encoded by a second polynucleotide construct.
[11] The polynucleotide construct of any one of embodiments 1-10, wherein expression of the Rep polypeptide and the Cap polypeptide are driven by native promoters.
[12] The polynucleotide construct of embodiment 11, wherein the native promoters comprise P5, P19, and P40.
[13] The polynucleotide construct of any one of embodiments 7-12, wherein the exon encodes a detectable marker.
[14] The polynucleotide construct of embodiment 13, wherein the detectable marker comprises a luminescent marker, a fluorescent marker, or radiolabel.
[15] The polynucleotide construct of embodiment 14, wherein the fluorescent marker is GFP, EGFP, RFP, CFP, BFP, YFP, or mCherry.
[16] A stable mammalian cell line, wherein cells of the cell line are suitable for conditional production of rAAV virions and wherein expression of a Rep polypeptide and a Cap polypeptide is inducible in the absence of a transfection agent.
[17] A stable mammalian cell line, wherein cells of the cell line are suitable for conditional production of rAAV virions and wherein expression of a Rep polypeptide and a Cap polypeptide is inducible in the absence of a plasmid.
[18] The stable mammalian cell line of any one of embodiments 16-17, wherein the cells comprise the polynucleotide construct of any one of embodiments 1-15 stably integrated into the cell's nuclear genome.
[19] The stable mammalian cell line of embodiment 18, wherein the cell line is monoclonal.
[20] The stable mammalian cell line of any one of embodiments 16-19, wherein cells of the cell line are capable of conditionally producing recombinant AAV (rAAV) virions upon addition of an excising element.
[21] A stable mammalian cell line, wherein a cell of the cell line comprises a stably integrated polynucleotide construct of any one of embodiments 1-14 and wherein the cell is capable of conditionally producing recombinant AAV (rAAV) virions upon addition of an excising element.
[22] The stable mammalian cell line of embodiment 21, wherein the excising element is a Cre polypeptide or a flippase.
[23] The stable mammalian cell line of embodiment 22, wherein the Cre polypeptide is encoded by a second polynucleotide construct.
[24] The stable mammalian cell line of any one of embodiments 21-23, wherein localization of the excising element is hormone regulated.
[25] The stable mammalian cell line of any one of embodiments 16-24, wherein the cell is capable of conditionally producing rAAV virions upon addition of at least two triggering agents.
[26] The stable mammalian cell line of embodiment 25, wherein the at least two triggering agents comprise doxycycline and tamoxifen.
[27] A polynucleotide construct coding for a mutated VA RNA, wherein the mutated VA RNA gene sequence comprises at least two mutations in an internal promoter.
[28] The polynucleotide construct of embodiment 27, wherein expression of VA RNA is driven by a U6 promoter.
[29] The polynucleotide construct of any one of embodiments 27-28, comprising upstream of the mutated VA RNA gene sequence, from 5′ to 3′:
   a) a first part of a U6 promoter sequence;
   b) a first lox sequence;
   c) a stuffer sequence;
   d) a second lox sequence;
   e) a second part of a U6 promoter sequence.
[30] The polynucleotide construct of embodiment 29, wherein the stuffer sequence is excisable by a Cre polypeptide.
[31] The polynucleotide construct of embodiment 30, wherein the Cre polypeptide is exogenously provided.
[32] The polynucleotide construct of embodiment 30, wherein the Cre polypeptide is encoded by the polynucleotide construct.
[33] The polynucleotide construct of embodiment 30, wherein the stuffer sequence is positioned between the first part of the U6 promoter sequence and the second part of the U6 promoter sequence.
[34]A polynucleotide construct coding for:
   a) one or more helper proteins;
   b) a self-excising element upstream of the one or more helper proteins; and
   c) an inducible promoter upstream of the self-excising element.
[35] The polynucleotide construct of embodiment 34, wherein expression of the self-excising element is driven by a Tet-On-3G system.
[36] The polynucleotide construct of any one of embodiments 34-35, wherein the polynucleotide construct further comprises a sequence that encodes a Tet responsive activator protein (Tet-on-3G).
[37] The polynucleotide construct of embodiment 36, wherein expression of Tet-On 3G activator protein is driven by an Elalpha promoter.
[38] The polynucleotide construct of embodiment 37, wherein, in the presence of a first triggering agent, Tet-On 3G activator protein binds to the inducible promoter.
[39] The polynucleotide construct of embodiment 38, wherein the inducible promoter is a Tet inducible promoter.
[40] The polynucleotide construct of any one of embodiments 34-39, wherein the self-excising element is a sequence encoding a Cre polypeptide.
[41] The polynucleotide construct of any one of embodiments 34-40, wherein expression of the self-excising element is hormone regulated.
[42] The polynucleotide construct of embodiment 41, wherein the self-excising element is expressed and self-excises only in the presence of a hormone.
[43] The polynucleotide construct of embodiment 42, wherein the hormone is tamoxifen.
[44] The polynucleotide construct of any one of embodiments 34-43, wherein the one or more adenoviral helper proteins comprise E2 and E4.
[45] The polynucleotide construct of any one of embodiments 34-44, wherein the polynucleotide construct further encodes a VA RNA.
[46] The polynucleotide construct of embodiment 45, wherein expression of VA RNA is driven by a Elalpha promoter.
[47] The polynucleotide construct of any one of embodiments 45-46, wherein the VA RNA is the mutated VA RNA of any one of embodiments 27-33.
[48] The polynucleotide construct of embodiment 37 or 46, wherein the Elalpha promoter comprises at least one mutation.
[49]A stable mammalian cell line, wherein cells of the cell line are suitable for conditional production of rAAV virions and wherein expression of one or more helper proteins is inducible in the absence of a transfection agent.
[50]A stable mammalian cell line, wherein cells of the cell line are suitable for conditional production of rAAV virions and wherein expression of one or more helper proteins is inducible in the absence of a plasmid.
[51]A stable mammalian cell line, wherein at least 70%, at least 80%, at least 90%, at least 95%, or 100% of the cells comprise the polynucleotide construct of any one of embodiments 27-48 stably integrated into the cell's nuclear genome.
[52] The stable mammalian cell line of any one of embodiments 49-51, wherein cells of the cell line comprise the polynucleotide construct of any one of embodiments 27-48 stably integrated into the cell's nuclear genome.
[53] The stable mammalian cell line of any one of embodiments 49-52, wherein a cell of the cell line is capable of conditionally producing recombinant AAV (rAAV) virions upon addition of at least two triggering agents.
[54]A stable mammalian cell line, wherein a cell of the cell line comprises a stably integrated polynucleotide construct of any one of embodiments 27-48, and wherein the cell is capable of conditionally producing recombinant AAV (rAAV) virions upon addition of at least two triggering agents.
[55] The stable mammalian cell line of any one of embodiments 53-54, wherein the at least two triggering agents comprise doxycycline and tamoxifen.
[56]A stable mammalian cell line, wherein:
   the cells are capable of conditionally producing recombinant AAV (rAAV) virions within which are packaged an expressible polynucleotide encoding a payload; and
   wherein a population of virions produced by the stable cell are more homogenous than a population of virions produced by an otherwise comparable mammalian cell producing rAAV virions upon transient transfection.
[57] The stable mammalian cell line of embodiment 56, wherein the population of virions produced by the stable cell has a ratio of viral genomes to transduction units of about 500:1 to 1:1.
[58] The stable mammalian cell line of embodiment 57, wherein the population of virions produced by the stable cell has a ratio of vector genomes to infectious unit of 100:1.
[59] The stable mammalian cell line of any one of embodiments 56-58, wherein production of virions is inducible upon addition of a triggering agent.
[60] The stable mammalian cell line of any one of embodiments 56-58, wherein production of virions is inducible upon addition of at least two triggering agents.
[61] The stable mammalian cell line of any one of embodiments 56-60, wherein a cell of the cell line comprises a stably integrated polynucleotide construct of any one of embodiments 1-15, 27-33, and 34-48.
[62] A stable mammalian cell line, wherein:
the cells are capable of conditionally producing recombinant AAV (rAAV) virions within which are packaged an expressible polynucleotide encoding a payload; and production of virions is inducible upon addition of a triggering agent.
[63] A stable mammalian cell line, wherein:
the cells are capable of conditionally producing recombinant AAV (rAAV) virions within which are packaged an expressible polynucleotide encoding a payload; and production of virions is not conditioned on the presence of a plasmid within the cell.
[64] The stable cell line of any of the previous embodiments, wherein expression of AAV Rep and Cap proteins is conditional.
[65] The stable cell line of any of the previous embodiments, wherein expression of AAV Rep and Cap proteins is conditioned on addition of at least a first expression triggering agent to the cell culture medium.
[66] The stable cell line of embodiment 65, wherein expression of AAV Rep and Cap proteins is conditioned on addition of a first expression triggering agent and a second expression triggering agent to the cell culture medium.
[67] The stable cell line of embodiment 66, wherein the cells do not express cytotoxic levels of Rep protein prior to addition of both the first expression and second triggering agents to the cell culture medium.
[68] The stable cell line of embodiment 67, wherein the cells do not express cytostatic levels of Rep protein prior to addition of both the first and second expression triggering agents to the cell culture medium.
[68] The stable cell line of embodiment 67 or 68, wherein the average concentration of Rep protein within the cells is less than the amount prior to addition of both of the first and second expression triggering agents to the cell culture medium.
[70] The stable cell line of any one of embodiments 65-69, wherein expression of Rep and Cap proteins becomes constitutive after addition of all of the at least first expression triggering agents to the cell culture medium.
[71] The stable cell line of any of embodiments 65-70, wherein expression of adenoviral helper proteins is conditional.
[72] The stable cell line of embodiment 71, wherein expression of adenoviral helper proteins is conditioned on addition of at least a third expression triggering agent to the cell culture medium.
[73] The stable cell line of embodiment 72, wherein the third expression triggering agent is the same as the first expression triggering agent.
[74] The stable cell line of embodiment 72 or 73, wherein expression of adenoviral helper proteins is conditioned on addition of a third expression triggering agent and a fourth expression triggering agent to the cell culture medium.
[75] The stable cell line of embodiment 74, wherein the fourth is the same as the second expression triggering agent.
[76] The stable cell line of embodiment 72 or 74, wherein the third expression triggering agent is the same as the first expression triggering agent and the fourth expression triggering agent is the same as the second expression triggering agent.
[77] The stable cell line of embodiment 76, wherein continued expression of adenoviral helper proteins following triggering of expression by contact of the cell with the at least third expression triggering agent requires the presence of only the third expression triggering agent in the cell culture medium.
[78] The stable cell line of embodiment 77, wherein the third triggering agent is the same as the first triggering agent.
[79] The stable cell line of any one of embodiments 71-78, wherein the adenoviral helper proteins include E2A and E4.
[80] The stable cell line of any one of embodiments 65-75, wherein the first expression triggering agent is a tetracycline.
[81] The stable cell line of embodiment 80, wherein the tetracycline is doxycycline.
[82] The stable cell line of any one of embodiments 74-76, wherein the fourth expression triggering agent is an estrogen receptor ligand.
[83] The stable cell line of embodiment 82, wherein the estrogen receptor ligand is a selective estrogen receptor modulator (SERM).
[84] The stable cell line of embodiment 83, wherein the estrogen receptor ligand is tamoxifen.
[85] The stable cell line of any of the previous embodiments, wherein expression of the therapeutic polynucleotide is conditioned on addition of at least a fifth expression triggering agent to the cell culture medium.
[86] The stable cell line of any one of embodiments 62-84, wherein expression of the therapeutic polynucleotide is not conditioned on addition of an expression triggering agent to the cell culture medium.
[87] The stable cell line of any of embodiments 62-86, wherein expression of Rep and Cap proteins, adenoviral helper proteins, and the expressible polynucleotide encoding a payload becomes constitutive after addition of only one expression triggering agent to the cell culture medium.
[88] The stable cell line of any of embodiments 62-87, wherein expression of Rep and Cap proteins and the adenoviral helper proteins becomes constitutive after addition of only one expression triggering agent to the cell culture medium.
[89] The stable cell line of embodiment 87 or 88, wherein the one expression triggering agent is the first expression triggering agent.
[90] The stable cell line of embodiment 89, wherein the first expression triggering agent is a tetracycline.
[91] The stable cell line of embodiment 90, wherein the first expression triggering agent is doxycycline.
[92] The stable cell line of any one of embodiments 62-91, wherein the nuclear genome of the cell comprises a plurality of integrated synthetic nucleic acid constructs.
[93]93. The stable cell line of embodiment 92, wherein the nuclear genome of the cell comprises at least two integrated synthetic constructs.
[94] The stable cell line of embodiment 93, wherein the nuclear genome of the cell comprises at least three integrated synthetic constructs.
[95] The stable cell line of any one of embodiments 92-94, wherein each of the plurality of synthetic nucleic acid constructs is separately integrated into the nuclear genome of the cell.
[96] The stable cell line of any one of embodiments 92-94, wherein only a single non-auxotrophic selection is required to maintain all of the plurality of synthetic nucleic acid constructs stably within the nuclear genome of the cells.
[97] The stable mammalian cell line of any one of embodiments 92-96, wherein:
   the first integrated synthetic construct comprises conditionally expressible AAV Rep and Cap coding sequences;
   the second integrated synthetic construct comprises a conditionally expressible Cre coding sequence and conditionally expressible adenoviral helper protein coding sequences; and
   the third integrated synthetic construct comprises expressible coding sequences for the expressible polynucleotide encoding a payload.
[98] The stable mammalian cell line of embodiment 97, wherein, prior to the cell being contacted with the first expression triggering agent, the Rep coding sequence of the first integrated construct is interrupted by an intervening spacer.
[99] The stable mammalian cell line of embodiment 98, wherein the intervening spacer comprises, from 5′ to 3′, a first spacer segment, a second spacer segment, and a third spacer segment.
[100] The stable mammalian cell line of embodiment 99, wherein the first spacer segment comprises a 5′ splice site (5′SS) 5′ to the first spacer element.
[101] The stable mammalian cell line of any one of embodiments 99-100, wherein the first spacer segment has a nucleic acid sequence having at least 80% identity to SEQ ID NO: 1.
[102] The stable mammalian cell line of embodiments 99-101, wherein the second spacer segment comprises a polynucleotide encoding a detectable protein marker flanked by lox sites.
[103] The stable mammalian cell line of embodiment 102, wherein the detectable protein marker is a fluorescent protein.
[104] The stable mammalian cell line of embodiment 103, wherein the fluorescent protein is a GFP.
[105] The stable mammalian cell line of embodiment 104, wherein the GFP is eGFP.
[106] The stable mammalian cell line of embodiment 99, wherein the second spacer segment further comprises a polyA sequence.
[107] The stable mammalian cell line of embodiment 106, wherein the polyA sequence comprises a rabbit beta globin (RBG) polyA.
[108] The stable mammalian cell line of embodiment 99, wherein the second spacer segment further comprises a first 3′ splice site (3′SS) between the first lox site and the polynucleotide encoding the protein marker.
[109] The stable mammalian cell line of any one of embodiments 98-101, wherein the second spacer segment comprises a nucleic acid sequence having at least 80% identity to SEQ ID NO: 2.
[110] The stable mammalian cell line of embodiment 99, wherein the third spacer segment further comprises a second 3′ splice site (3′SS).
[111] The stable mammalian cell line of embodiment 110, wherein the second 3′ splice site is positioned 3′ to the second lox site.
[112] The stable mammalian cell line of any one of embodiments 98-111, wherein the third spacer segment comprises a nucleic acid sequence having at least 80% identity to SEQ ID NO: 3.
[113] The stable mammalian cell line of any one of embodiments 98-112, wherein the Rep coding sequence is operatively linked to an endogenous P5 promoter.
[114] The stable mammalian cell line of any one of embodiments 98-113, wherein the Rep coding sequence is operatively linked to an endogenous P19 promoter.
[115] The stable mammalian cell line of any one of embodiments 98-114, wherein the intervening spacer is inserted into the Rep coding sequence at a position downstream of the P19 promoter.
[116] The stable mammalian cell line of any one of embodiments 98-115, wherein the Rep coding sequence is 5′ to the Cap coding sequence.
[117] The stable mammalian cell line of embodiment 116, wherein the Cap coding sequence is operatively linked to an endogenous P40 promoter.
[118] The stable mammalian cell line of any one of embodiments 98-117, wherein the first integrated construct further comprises a first mammalian cell selection element.
[119] The stable mammalian cell line of embodiment 118, wherein the first mammalian cell selection element is an auxotrophic selection element.
[120] The stable mammalian cell line of embodiment 119, wherein the auxotrophic selection element encodes an active protein.
[121] The stable mammalian cell line of embodiment 120, wherein the active protein is DHFR.
[122] The stable mammalian cell line of any one of embodiments 119-121, wherein the auxotrophic selection coding sequence encodes an inactive protein that requires expression of a second auxotrophic selection coding sequence for activity.
[123] The stable mammalian cell line of embodiment 122, wherein the second auxotrophic selection coding sequence codes for DHFR Z-Cter (SEQ ID NO: 5).
[124] The stable mammalian cell line of any one of embodiments 97-123, wherein, prior to the cell being contacted with the first expression triggering agent, the second integrated construct comprises, from 5′ to 3′, an inducible promoter, a Cre coding sequence, a first polyA sequence, adenoviral helper protein coding sequences, a second polyA sequence, a constitutive promoter, a coding sequence for a protein that is responsive to the first expression triggering agent, and a second mammalian cell selection element.
[125] The stable mammalian cell line of embodiment 124, wherein the Cre coding sequence is operatively linked to the inducible promoter.
[126] The stable mammalian cell line of embodiment 125, wherein the inducible promoter comprises an element responsive to the third expression triggering agent.
[127] The stable mammalian cell line of embodiment 125, wherein the inducible promoter comprises a plurality of tetracycline (Tet) operator elements capable of binding to a Tet responsive activator protein in the presence of a tetracycline.
[128] The stable mammalian cell line of any one of embodiments 124-127, further comprises an element responsive to the fourth expression triggering agent.
[129] The stable mammalian cell line of embodiment 128, wherein the fourth expression triggering agent-responsive element comprises a plurality of hormone-response elements.
[130] The stable mammalian cell line of embodiment 129, wherein the hormone-response elements are estrogen responsive elements (EREs).
[131] The stable mammalian cell line of any one of embodiments 124-130, wherein the third expression triggering element is the same as the first expression triggering element, and the fourth expression triggering element is the same as the second expression triggering element.
[132] The stable mammalian cell line of embodiment 124, wherein the Cre coding sequence is flanked by a first lox site and a second lox site.
[133] The stable mammalian cell line of embodiment 124, wherein the inducible promoter comprises a plurality of Tet operator elements capable of binding to a Tet responsive activator protein in the presence of a first expression triggering agent.
[134] The stable mammalian cell line of any one of embodiments 124-133, wherein the adenoviral helper protein coding sequences encode E2A and E4.
[135] The stable mammalian cell line of any one of embodiments 124-133, wherein the coding sequence for the first expression triggering agent-responsive protein is operatively linked to a CMV promoter.
[136] The stable mammalian cell line of any one of embodiments 124-135, wherein the coding sequence for the first expression triggering agent-responsive protein comprises a coding sequence for the Tet responsive activator protein (Tet-on-3G).
[137] The stable mammalian cell line of any one of embodiments 124-136, wherein the second mammalian cell selection element confers antibiotic resistance.
[138] The stable mammalian cell line of embodiment 137, wherein the antibiotic resistance conferring element is a blasticidin resistance gene.
[139] The stable mammalian cell line of any one of embodiments 98-138, wherein the third integrated synthetic construct comprises coding sequence for the expressible polynucleotide payload, and a third mammalian cell selection element.
[140] The stable mammalian cell line of embodiment 139, wherein the expressible polynucleotide payload encodes a guide RNA for RNA editing.
[141] The stable mammalian cell line of embodiment 139, wherein the expressible polynucleotide payload encodes a guide RNA for Cas protein-directed DNA editing.
[142] The stable mammalian cell line of embodiment 139, wherein the expressible polynucleotide payload encodes a protein.
[143] The stable mammalian cell line of embodiment 139, wherein the expressible polynucleotide payload comprises a gene for replacement gene therapy.
[144] The stable mammalian cell line of embodiment 139, wherein the expressible polynucleotide payload comprises a homology construct for homologous recombination.
[145] The stable mammalian cell line of embodiment 139, wherein the expressible polynucleoide payload encodes a therapeutic antibody.
[146] The stable mammalian cell line of embodiment 139, wherein the expressible payload polynucleotide encodes a vaccine immunogen.
[147] The stable mammalian cell line of any one of embodiments 139-146, wherein the third mammalian cell selection element is an auxotrophic selection element.
[148] The stable mammalian cell line of embodiment 147, wherein the auxotrophic selection element encodes an active protein.
[149] The stable mammalian cell line of embodiment 148, wherein the active protein is DHFR.
[150] The stable mammalian cell line of any one of embodiments 147-149, wherein the auxotrophic selection coding sequence encodes an inactive protein that requires expression of a second auxotrophic selection coding sequence for activity.
[151] The stable mammalian cell line of embodiment 150, wherein the second auxotrophic selection coding sequence codes for DHFR Z-Nter (SEQ ID NO: 4).
[152] The stable mammalian cell line of embodiment 123 or 151, wherein DHFR selection comprises cell growth in media lacking hypoxanthine-thymidine.
[153] The stable mammalian cell line of any one of the proceeding embodiments, wherein the mammalian cell line expresses adenovirus helper proteins E1A and E1B and is a human embryonic kidney (HEK) 293 cell line, a human HeLa cell line, or a Chinese hamster ovary (CHO) cell line.
[154] The stable mammalian cell line of any one of the proceeding embodiments, wherein the mammalian cell line is a HEK293 cell line.
[155] The stable mammalian cell line of any one of the proceeding embodiments, wherein the mammalian cell line expresses adenovirus helper proteins E1A and E1B.
[156] A method of producing rAAV, comprising adding all of the at least first and at least second expression triggering agents to the stable mammalian cell line of any one of embodiments 62-155, in culture.
[157] The method of embodiment 156, further comprising a later step of culturing the stable mammalian cell line only in the presence of the first expression triggering agent.
[158] The method of embodiment 157, further comprising purifying rAAV from culture medium.
[159] An rAAV product made by the process of embodiments 156-158.
[160] A method of treating a condition or disorder, comprising administering a therapeutically effective amount of the rAAV product according to embodiment 159 to a patient in need thereof.
[161] A method of treating a monogenic disorder, comprising administering a therapeutically effective amount of the rAAV product according to embodiment 159 to a patient having a monogenic disorder, wherein expression of the rAAV payload improves the symptoms of the disorder.
[162] The stable cell line of any one of embodiments 27-48, 54-55, and 71, further comprising an inducible VA RNA.
[162] The stable cell line of embodiment 162, wherein the VA RNA is encoded by a construct comprising at least one mutation in a promoter operatively linked to the VA RNA encoding sequence.
164. The stable cell line of embodiment 163, wherein the at least one mutation is in the A Box promoter region.
165. The stable cell line of embodiment 163, wherein the at least one mutation is in the B Box promoter region.
166. The stable cell line of embodiment 164, further comprising at least one mutation in B Box promoter region.
167. The stable cell line of embodiment 163, wherein the VA RNA is encoded by a construct comprising a deletion of from about 5-10 nucleotides in the promoter region.
168. The stable cell line of any of embodiments 162-167, wherein the inducible adenoviral RNA is operably linked to a U6 promoter segment.
169. The stable cell line of embodiment 168, wherein the U6 promoter segment comprises a stuffer or filler sequence that is flanked by a first lox site and a second lox site.

### 6.16. Numbered Embodiments #2

[1] A packaging cell line, comprising:
   a first integrated polynucleotide construct and
   a second integrated polynucleotide construct,
wherein
   the first integrated polynucleotide construct comprises conditionally expressible AAV Rep protein and AAV Cap protein coding sequences;
the second integrated polynucleotide construct comprises one or more conditionally expressible adenoviral helper protein coding sequences, and optionally a conditionally expressible VA RNA coding sequence; and
   the expression of Rep protein, Cap protein, and the one or more adenoviral helper proteins is inducible in the absence of a plasmid.
[2] The packaging cell line of embodiment 1, wherein the expression of Rep protein, Cap protein, and adenoviral helper proteins is inducible in the absence of a transfection agent.
[3] The packaging cell line of embodiment 1 or 2, wherein the first integrated polynucleotide construct comprises
   a) a first part of an AAV Rep protein coding sequence,
   b) a second part of an AAV Rep protein coding sequence
   c) an excisable element between the first part of the AAV Rep protein coding sequence and the second part of the AAV Rep protein coding sequence, and
   d) an AAV Cap protein coding sequence.
[4] The packaging cell line of embodiment 3, wherein the excisable element comprises
   a) a first spacer segment comprising a first intron,
   b) a second spacer segment comprising a coding sequence of a detectable marker, and
   c) a third spacer segment comprising a second intron, and
   wherein the first spacer segment and the third spacer segment are capable of being excised by endogenous cellular machinery.
[5] The packaging cell line of embodiment 4, wherein the detectable marker is a fluorescent marker or a luminescent marker.
[6] The packaging cell line of embodiment 4 or 5, wherein the second spacer segment is capable of being excised by a Cre polypeptide.
[7] The packaging cell line of any of the preceding embodiments, wherein the expression of the AAV Rep protein and the AAV Cap protein is driven by native promoters.
[8] The packaging cell line of embodiment 7, wherein the native promoters comprise P5, P19, and P40.
[9] The packaging cell line of any of the preceding embodiments, wherein the second integrated polynucleotide construct comprises
   a) one or more adenoviral helper protein coding sequences,
   b) a conditionally self-excising element, and
   c) an inducible promoter, and
   wherein the expression of the one or more adenoviral helper protein coding sequences is under the control of the conditionally self-excising element and the inducible promoter.
[10] The packaging cell line of embodiment 9, wherein the one or more adenoviral helper proteins comprise E2A and E4, and optionally, wherein E2A comprises FLAG tag.
[11] The packaging cell line of embodiment 9 or 10, wherein the self-excising element encodes a Cre polypeptide.
[12] The packaging cell line of any one of embodiments 9 to 11, wherein the polypeptide encoded by the self-excising element translocates to nucleus and self-excises only in the presence of a triggering agent.
[13] The packaging cell line of embodiment 12, wherein the triggering agent is tamoxifen.
[14] The packaging cell line of any one of embodiments 9 to 13, wherein the inducible promoter is a Tet inducible promoter.
[15] The packaging cell line of any one of embodiments 9 to 14, wherein the second integrated polynucleotide construct further comprises a sequence that encodes a Tet responsive activator protein (Tet-on-3G).
[16] The packaging cell line of embodiment 15, wherein the expression of Tet-On 3G activator protein is driven by an Elalpha promoter.
[17] The packaging cell line of any of the preceding embodiments, wherein the second integrated polynucleotide construct further comprises a segment encoding a VA RNA sequence.
[18] The packaging cell line of embodiment 17, wherein the expression of VA RNA is constitutive.
[19] The packaging cell line of embodiment 17, wherein the expression of VA RNA is inducible.
[20] The packaging cell line of embodiment 19, wherein the VA RNA sequence comprises a mutation in the VA RNA internal promoter.
[21] The packaging cell line of embodiment 20, wherein the VA RNA sequence comprises G16A and G60A mutations.
[22] The packaging cell line of any one of embodiment 19 to 21, wherein the expression of VA RNA is driven by a U6 promoter.
[23] The packaging cell line of embodiment 22, wherein
   the U6 promoter comprises
   a) a first part of a U6 promoter sequence,
   b) a stuffer sequence, and
   c) a second part of a U6 promoter sequence, and
   wherein the stuffer sequence is capable of being excised by a Cre polypeptide.
[24] The packaging cell line of any of the preceding embodiments, wherein the cell line expresses adenoviral helper proteins E1A and E1B.
[25] The packaging cell line of any of the preceding embodiments, wherein the AAV Cap protein is a capsid selected from the group consisting of AAV1, AAV2, AAV3, AAV4, AAV5, AAV6, AAV7, AAV8, AAV9, AAV 10, AAV11, AAV 12, AAV13, AAV 14, AAV 15 and AAV 16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16 and AAVhu68.
[26] The packaging cell line of embodiment 25, wherein the AAV Cap protein is an AAV5 capsid protein that comprises one or more mutations or insertions.
[27] A method of producing recombinant AAV (rAAV) virions, the method comprising: transfecting the packaging cell line of any of the preceding embodiments with a polynucleotide construct that comprises a packageable coding sequence for a payload, and inducing the expression of Rep protein, Cap protein, one or more adenoviral helper proteins, and optionally VA RNA, whereby the packageable coding sequence for a payload is encapsidated in an AAV capsid.
[28] The method of embodiment 27, further comprising the subsequent step of purifying the rAAV virions from cell culture media and/or cell lysate.
[29] A production cell line, comprising:
   the packaging cell line of any one of embodiments 1 to 26 and
   a polynucleotide construct comprising a packageable coding sequence for a payload.
[30] A production cell line, comprising:
   a first integrated polynucleotide construct,
   a second integrated polynucleotide construct, and
   a third integrated polynucleotide construct,
wherein
   the first integrated polynucleotide construct comprises conditionally expressible AAV Rep protein and AAV Cap protein coding sequences;
   the second integrated polynucleotide construct comprises one or more conditionally expressible adenoviral helper protein coding sequences, and optionally a conditionally expressible VA RNA coding sequence;
   the third integrated polynucleotide construct comprises a packageable coding sequence for a payload; and
   the production of recombinant AAV (rAAV) virions containing the coding sequence for the payload is inducible in the absence of a plasmid.
[31] The production cell line of embodiment 30, wherein the production of rAAV virions containing the coding sequence for the payload is inducible in the absence of a transfection agent.
[32] The production cell line of embodiment 30 or 31, wherein the first integrated polynucleotide construct comprises
   a) a first part of an AAV Rep protein coding sequence,
   b) a second part of an AAV Rep protein coding sequence,
   c) an excisable element between the first part of the AAV Rep protein coding sequence and the second part of the AAV Rep protein coding sequence, and
   d) an AAV Cap protein coding sequence.
[33] The production cell line of embodiment 32, wherein the excisable element comprises
   a) a first spacer segment comprising a first intron,
   b) a second spacer segment comprising a coding sequence of a detectable marker, and
   c) a third spacer segment comprising a second intron, and
   wherein the first spacer segment and the third spacer segment are capable of being excised by endogenous cellular machinery.
[34] The production cell line of embodiment 33, wherein the detectable marker is a fluorescent marker or a luminescent marker.
[35] The production cell line of embodiment 33 or 34, wherein the second spacer segment is capable of being excised by a Cre polypeptide.
[36] The production cell line of any one of embodiments 30 to 35, wherein the expression of the AAV Rep protein and the AAV Cap protein is driven by native promoters.
[37] The production cell line of embodiment 36, wherein the native promoters comprise P5, P19, and P40.
[38] The production cell line of any one of embodiments 30 to 37, wherein the second integrated polynucleotide construct comprises
   a) one or more adenoviral helper protein coding sequences,
   b) a conditionally self-excising element, and
   c) an inducible promoter, and
   wherein the expression of the one or more adenoviral helper protein coding sequences is under the control of the conditionally self-excising element and the inducible promoter.
[39] The production cell line of embodiment 38, wherein the one or more adenoviral helper proteins comprise E2A and E4.
[40] The production cell line of embodiment 38 or 39, wherein the self-excising element encode a Cre polypeptide.
[41] The production cell line of any one of embodiments 38 to 40, wherein the polypeptide encoded by the self-excising element translocates to nucleus and self-excises only in the presence of a triggering agent.
[42] The production cell line of embodiment 41, wherein the triggering agent is tamoxifen.
[43] The production cell line of any one of embodiments 38 to 42, wherein the inducible promoter is a Tet inducible promoter.
[44] The production cell line of any one of embodiments 38 to 43, wherein the second integrated polynucleotide construct further comprises a sequence that encodes a Tet responsive activator protein (Tet-on-3G).
[45] The production cell line of embodiment 44, wherein the expression of Tet-On 3G activator protein is driven by an Elalpha promoter.
[46] The production cell line of any one of embodiments 30 to 45, wherein the second integrated polynucleotide construct further comprises a segment encoding a VA RNA sequence.
[47] The production cell line of embodiment 46, wherein the expression of VA RNA is constitutive.
[48] The production cell line of embodiment 46, wherein the expression of VA RNA is inducible.
[49] The production cell line of embodiment 48, wherein the VA RNA sequence comprises a mutation in the VA RNA internal promoter.
[50] The production cell line of embodiment 49, wherein the VA RNA sequence comprises G16A and G60A mutations.
[51] The production cell line of any one of embodiments 48 to 50, wherein the expression of VA RNA is driven by a U6 promoter.
[52] The production cell line of embodiment 51, wherein
   the U6 promoter comprises
   a) a first part of a U6 promoter sequence,
   b) a stuffer sequence, and
   c) a second part of a U6 promoter sequence, and
   wherein the stuffer sequence is capable of being excised by a Cre polypeptide.
[53] The production cell line of any one of embodiments 30 to 52, wherein the cell line expresses adenoviral helper proteins E1A and E1B.
[54] The production cell line of any one of embodiments 30 to 53, wherein a serotype of the AAV Cap protein is the serotype selected from the group consisting of AAV1, AAV2, AAV3, AAV4, AAV5, AAV6, AAV7, AAV8, AAV9, AAV 10, AAV11, AAV 12, AAV13, AAV 14, AAV 15 and AAV 16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16 and AAVhu68.
[55] The production cell line of embodiment 54, wherein the AAV Cap protein comprises a VP1 capsid protein that comprises one or more mutations or insertions, and optionally, wherein the serotype is AAV5 or AAV9.
[56] A method of producing recombinant AAV (rAAV) virions, the method comprising: culturing the production cell line of any one of embodiments 29 to 55, and inducing the expression of Rep protein, Cap protein, one or more adenoviral helper proteins, and optionally VA RNA.
[57] The method of embodiment 56, further comprising the subsequent step of purifying the rAAV virions from cell culture media or cell lysate.
[58] A production cell line for producing recombinant AAV (rAAV) virions, wherein the production of rAAV virions is inducible in the absence of a plasmid, and wherein following induction, the production cell line is capable of producing a prepurification rAAV yield of no less than 1×10¹⁴ viral genome (vg)/L.
[59] The production cell line of embodiment 58, wherein the production of rAAV virions is inducible in the absence of a transfection agent.
[60] The production cell line of embodiment 58 or 59, wherein the production of rAAV virions is inducible in the absence of herpes simplex virus, baculovirus, and adenovirus.
[61] The production cell line of any one of embodiments 58 to 60, wherein following induction, the production cell line is capable of producing a prepurification rAAV full capsid to empty capsid ratio of no less than 0.5.
[62] The production cell line of any one of embodiments 58 to 61, wherein following induction, culturing, and downstream processing, the production cell line is capable of producing a postpurification rAAV yield of no less than 1×10¹⁴ viral genome (vg)/L without ultracentrifugation.
[63] The production cell line of any one of embodiments 58 to 62, wherein following induction, culturing, and downstream processing, the production cell line is capable of producing a postpurification rAAV full capsid to empty capsid ratio of no less than 0.5 without ultracentrifugation.
[64] The production cell line of any one of embodiments 58 to 63, wherein following induction, the production cell line is capable of producing rAAV virions that are more homogenous than a population of rAAV virions produced by an otherwise comparable cell line following transient triple transfection.
[65] A production cell line for producing recombinant AAV (rAAV) virions,
   wherein the production of rAAV virions is inducible in the absence of a plasmid, and
   wherein following induction, the production cell line is capable of producing rAAV virions having a prepurification rAAV full capsid to empty capsid ratio of no less than 0.5.
[66] The production cell line of embodiment [65], wherein the production cell line is capable of producing rAAV virions that, following purification and administration to a subject, induce a lower titer of neutralizing antibodies per administered rAAV viral genome compared to a population of rAAV virions that have the same capsid and rAAV viral genome and are produced by an otherwise comparable cell line following transient triple transfection and that are administered by the same route of administration to a comparable subject.
[67] The production cell line of embodiment [65] or [66], wherein the production cell line is capable of producing rAAV virions that, following purification and administration to a subject, induce fewer and/or lower intensity adverse effects per administered rAAV viral genome compared to a population of rAAV virions that have the same capsid and rAAV viral genome and are produced by an otherwise comparable cell line following transient triple transfection and that are administered by the same route of administration to a comparable subject.
[68] The production cell line of embodiment [67], wherein the adverse effect is selected from the group consisting of: liver dysfunction, liver inflammation, gastrointestinal infection, vomiting, bacterial infection, sepsis, increases in troponin levels, decreases in red blood cell counts, decreases in platelet counts, activation of the complement immune system response, acute kidney injury, cardio-pulmonary insufficiency, and death.
[69] The production cell line of embodiment [67], wherein the adverse effect is an increase in serum levels of one or more of interferon gamma (IFNγ), interleukin 1β (IL-1β), and interleukin 6 (IL-6).
[70] The production cell line of any one of embodiments [65] to [69], wherein the production cell line is capable of producing rAAV virions that have an effective dose of less than 1×10¹⁴ viral particles (vp)/kg.
[71] The production cell line of any one of embodiments [65] to [70], wherein the production cell line is capable of producing rAAV virions that can be administered to a patient more than once.
[72] The production cell line of any one of embodiments [65] to [71], wherein the production cell line is capable of producing rAAV virions that have a reduced prepurification quality variability compared to a population of rAAV virions produced by an otherwise comparable cell line following transient triple transfection.
[73] The production cell line of any one of embodiments [65] to [71], wherein the production cell line is capable of producing rAAV virions that have a reduced postpurification quality variability compared to a population of rAAV virions produced by an otherwise comparable cell line following transient triple transfection.
[74] The production cell line of embodiment [72] or [73], wherein the purification quality variability is selected from: viral genome to viral particle ratio variability, yield variability, potency variability, purity variability, DNA content variability, and capsid variability.
[75] A production cell line for producing recombinant AAV (rAAV) virions,
   wherein the production of rAAV virions is inducible in the absence of a plasmid, and
   wherein following induction, the production cell line is capable of producing rAAV virions that have an increased batch consistency of rAAV having a predetermined number of viral genomes (VG) compared to a population of rAAV virions that have the same capsid and VG and are produced by an otherwise comparable cell line following transient triple transfection.
[76] The production cell line of embodiment [75], wherein the batch consistency is measured by a variation in the number of viral particles (VP) between batches of rAAV having a predetermined number of viral genomes (VG) from different batches.
[77] The production cell line of embodiment [75] or [76], wherein the batch consistency is increased by 2-fold compared to a population of rAAV virions that have the same capsid and VG and are produced by an otherwise comparable cell line following transient triple transfection.
[78] The production cell line of embodiment [77], wherein the batch consistency is increased by 5-fold compared to a population of rAAV virions that have the same capsid and VG and are produced by an otherwise comparable cell line following transient triple transfection.
[79] The production cell line of embodiment [78], wherein the batch consistency is increased by 10-fold compared to a population of rAAV virions that have the same capsid and VG and are produced by an otherwise comparable cell line following transient triple transfection.
[80] The production cell line of any one of embodiments [75] to [79], wherein the number of viral particles (VP) between batches of rAAV having a predetermined number of viral genomes (VG) varies by no more than 20%.
[81] The production cell line of embodiment [80], wherein the number of viral particles (VP) between batches of rAAV having a predetermined number of viral genomes (VG) varies by no more than 10%.
[82] The production cell line of embodiment [81], wherein the number of viral particles (VP) between batches of rAAV having a predetermined number of viral genomes (VG) varies by no more than 5%.
[83] A cell culture composition comprising:
   a) suspension-adapted mammalian cells,
   b) serum-free cell culture media, and
   c) recombinant AAV (rAAV) virions,
   wherein the cell culture composition is free of herpes simplex virus, baculovirus, and adenovirus, and
   wherein the cell culture composition is free of plasmid and transfection agent.
[84] The cell culture composition of embodiment 83, wherein the cell culture composition is free of polyethylenimine (PEI).
[85] The cell culture composition of embodiment 83 or 84, wherein the suspension-adapted mammalian cells are suspension-adapted HEK293 cells or derivatives thereof.
[86] The cell culture composition of any one of embodiments 83 to 85, wherein the suspension-adapted mammalian cells are cells of the packaging cell line of any one of embodiments 1 to 26 or cells of the production cell line of any one of embodiments 29 to 55.
[87] The cell culture composition of any one of embodiments 83 to 86, wherein the cell culture composition has a prepurification rAAV concentration of greater than 1×10¹⁴ viral genome (vg)/L.
[88] The cell culture composition of any one of embodiments 83 to 87, wherein the cell culture composition has a prepurification rAAV viral particle to viral genome (VG) ratio of no less than 0.5.
[89] A bioreactor containing the cell culture composition of any one of embodiments 59 to 88.
[90] The bioreactor of embodiment 89, wherein the bioreactor is a 1 L bioreactor.
[91] The bioreactor of embodiment 90, wherein the bioreactor has a total rAAV yield of greater than 1×10¹⁴ viral genome (vg).
[92] The bioreactor of embodiment 89, wherein the bioreactor is a 5 L bioreactor.
[93] The bioreactor of embodiment 92, wherein the bioreactor has a total rAAV yield of greater than 5×10¹⁴ viral genome (vg).
[94] The bioreactor of embodiment 89, wherein the bioreactor is a 50 L bioreactor.
[95] The bioreactor of embodiment 94, wherein the bioreactor has a total rAAV yield of greater than 5×10¹⁵ viral genome (vg).
[96] The bioreactor of embodiment 89, wherein the bioreactor is a 100 L bioreactor.
[97] The bioreactor of embodiment 96, wherein the bioreactor has a total rAAV yield of greater than 1×10¹⁶ viral genome (vg).
[98] The bioreactor of embodiment 89, wherein the bioreactor is a 500 L bioreactor.
[99] The bioreactor of embodiment 98, wherein the bioreactor has a total rAAV yield of greater than 5×10¹⁶ viral genome (vg).
[100] The bioreactor of embodiment 89, wherein the bioreactor is a 2000 L bioreactor.
[101] The bioreactor of embodiment 100, wherein the bioreactor has a total rAAV yield of greater than 2×10¹⁷ viral genome (vg).
[102] The bioreactor of any one of embodiments 89 to 101, wherein the bioreactor is a single use bioreactor.
[103] A method of producing recombinant AAV (rAAV) virions using a bioreactor, the method comprising:
   culturing the packaging cell line of any one of embodiments 1 to 26 in the bioreactor,
   transfecting the packaging cell line with a polynucleotide construct that comprises a packageable coding sequence for a payload, and
   inducing the expression of Rep protein, Cap protein, one or more adenoviral helper proteins, and optionally VA RNA.
[104] A method of producing recombinant AAV (rAAV) virions using a bioreactor, the method comprising:
   culturing the production cell line of any one of embodiments 29 to 55 in the bioreactor, and
   inducing the expression of Rep protein, Cap protein, one or more adenoviral helper proteins, and optionally VA RNA.
[105] The method of embodiment 103 or 104, further comprising the subsequent step of purifying the rAAV virions from the cell culture composition.
[106] A pharmaceutical composition, comprising:
   cGMP grade rAAV virions produced by the packaging cell line, the production cell line, the cell culture composition, the bioreactor, or the method of any of the preceding embodiments, and
   a pharmaceutically acceptable carrier.
[107] The pharmaceutical composition of embodiment 106, wherein the pharmaceutical composition is free of plasmid and transfection agent.
[108] The pharmaceutical composition of embodiment 106 or 107, wherein the pharmaceutical composition is free of herpes simplex virus, baculovirus, and adenovirus.
[109] The pharmaceutical composition of any one of embodiments 106 to 108, wherein the pharmaceutical composition is free of herpes simplex virus, baculovirus, and adenovirus DNA.
[110] The pharmaceutical composition of any one of embodiments 106 to 109, wherein the pharmaceutical composition has a postpurification rAAV concentration of at least 1×10¹¹ viral genome (vg)/mL.
[111] The pharmaceutical composition of any one of embodiments 106 to 110, wherein the pharmaceutical composition has a postpurification rAAV viral particle to viral genome (VG) ratio of no less than 0.8.
[112] A pharmaceutical unit dose comprising the pharmaceutical composition of any one of embodiments 106 to 111.
[113] The pharmaceutical unit dose of embodiment 112, wherein the number of viral particles between doses of rAAV having a predetermined number of viral genomes varies by no more than 20%.
[114] The pharmaceutical unit dose of embodiment [113], wherein the number of viral particles between doses of rAAV having a predetermined number of viral genomes varies by no more than 10%.
[115] The pharmaceutical unit dose of embodiment [114], wherein the number of viral particles between doses of rAAV having a predetermined number of viral genomes varies by no more than 5%.
[116] A pharmaceutical unit dose comprising: at least 1×10¹¹ rAAV viral genome at a postpurification rAAV full capsid to empty capsid ratio of no less than 0.8 in 2 mL.
[117] A method for reducing the immunogenicity of a dose of rAAV having a predetermined number of viral genomes (VG) as compared to the same rAAV VG dose prepared by transient triple transfection, the method comprising:
   producing the rAAV in the packaging cell line of any one of embodiments 1 to 26, the production cell line of any one of embodiments 29 to 55 and 58 to 74, or the cell culture composition of any one of embodiments 83 to 88, or the bioreactor of any one of embodiments 89 to 94, or using the production method of any one of embodiments 27, 28, 56, 57, and 103 to 105.
[118] A method for reducing the number or intensity of adverse effects caused by administering a dose of rAAV having a predetermined number of viral genomes (VG) as compared to the same rAAV VG dose prepared by transient triple transfection, the method comprising:
   producing the rAAV in the packaging cell line of any one of embodiments 1 to 26, the production cell line of any one of embodiments 29 to 55 and 58 to 74, or the cell culture composition of any one of embodiments 83 to 88, or the bioreactor of any one of embodiments 89 to 94, or using the production method of any one of embodiments 27, 28, 56, 57, and 103 to 105.
[119] The method of embodiment [118], wherein the adverse effect is selected from the group consisting of: liver dysfunction, liver inflammation, gastrointestinal infection, vomiting, bacterial infection, sepsis, increases in troponin levels, decreases in red blood cell counts, decreases in platelet counts, activation of the complement immune system response, acute kidney injury, cardio-pulmonary insufficiency, and death.
[120] The method of embodiment [118], wherein the adverse effect is an increase in serum levels of one or more of interferon gamma (IFNγ), interleukin 1β (IL-1β), and interleukin 6 (IL-6).
[121] A method of administering a dose of rAAV having a predetermined number of viral genomes (VG) to a subject with reduced production of neutralizing antibodies by the subject as compared to production of neutralizing antibodies after administration of the same rAAV VG dose prepared by transient triple transfection, the method comprising:
   administering a first dose of rAAV produced in the packaging cell line of any one of embodiments 1 to 26, the production cell line of any one of embodiments 29 to 55 and 58 to 74, or the cell culture composition of any one of embodiments 83 to 88, or the bioreactor of any one of embodiments 89 to 94, or using the production method of any one of embodiments 27, 28, 56, 57, and 103 to 105.
[122] The method of embodiment [121], further comprising:
   administering at least a second dose of rAAV produced in the packaging cell line of any one of embodiments 1 to 26, the production cell line of any one of embodiments 29 to 55 and 58 to 74, or the cell culture composition of any one of embodiments 83 to 88, or the bioreactor of any one of embodiments 89 to 94, or using the production method of any one of embodiments 27, 28, 56, 57, and 103 to 105.
[123] A method of administering a dose of rAAV having a predetermined number of viral genomes (VG) to a subject with reduced number or intensity of adverse effects as compared to administration of the same rAAV VG dose prepared by transient triple transfection, the method comprising:
   administering a dose of rAAV produced in the packaging cell line of any one of embodiments 1 to 26, the production cell line of any one of embodiments 29 to 55 and 58 to 74, or the cell culture composition of any one of embodiments 83 to 88, or the bioreactor of any one of embodiments 89 to 94, or using the production method of any one of embodiments 27, 28, 56, 57, and 103 to 105.
[124] The method of embodiment [123], wherein the adverse effect is selected from the group consisting of: liver dysfunction, liver inflammation, gastrointestinal infection, vomiting, bacterial infection, sepsis, increases in troponin levels, decreases in red blood cell counts, decreases in platelet counts, activation of the complement immune system response, acute kidney injury, cardio-pulmonary insufficiency, and death.
[125] The method of embodiment [123], wherein the adverse effect is an increase in serum levels of one or more of interferon gamma (IFNγ), interleukin 1β (IL-1β), and interleukin 6 (IL-6).
[126] A method for repeatedly administering a dose of rAAV to a subject in need thereof, the method comprising:
   administering a first dose of rAAV by a first route of administration, wherein the rAAV are produced in the packaging cell line of any one of embodiments 1 to 26, the production cell line of any one of embodiments 29 to 55 and 58 to 74, or the culture composition of any one of embodiments 83 to 88, or the bioreactor of any one of embodiments 89 to 94, or using the production method of any one of embodiments 27, 28, 56, 57, and 103 to 105, and then
   administering at least a second dose of rAAV by either the first route of administration or a second route of administration, wherein the rAAV are produced in the packaging cell line of any one of embodiments 1 to 26, the production cell line of any one of embodiments 29 to 55 and 58 to 74, or the cell culture composition of any one of embodiments 83 to 88, or the bioreactor of any one of embodiments 89 to 94, or using the production method of any one of embodiments 27, 28, 56, 57, and 103 to 105,
   wherein the therapeutic effect of the payload varies by less than 10%, 20%, 30%, 40%, or 50% after administering the first dose compared to after administering at least the second dose.
[127] The method of embodiment 126, wherein the therapeutic effect of the payload varies by less than 10%, 20%, 30%, 40%, or 50% after administering the first dose compared to after administering at least the second dose.
[128] A method for producing a plurality of rAAV batches having a number of viral genomes (VG) as compared to the same rAAV VG batches prepared by transient triple transfection, the method comprising:
   producing the plurality of rAAV batches in the packaging cell line of any one of embodiments 1 to 26, the production cell line of any one of embodiments 29 to 55 and 58 to 74, or the cell culture composition of any one of embodiments 83 to 88, or the bioreactor of any one of embodiments 89 to 94, or using the production method of any one of embodiments 27, 28, 56, 57, and 103 to 105.
[129] The method of embodiment 128, wherein the number of viral genomes (VG) between the plurality of rAAV batches varies by no more than 50%, 40%, 30%, 20%, 10%, or 5%.
[130] The method of embodiment 128 or embodiment 129, wherein the number of viral particles between the plurality of rAAV batches varies by no more than 50%, 40%, 30%, 20%, 10%, or 5%.
[131] The method of embodiment any one of embodiments 128 to 130, wherein the number of viral particles (VP) or the number of viral genomes is from the plurality of batches prepurification.
[132] A method for increasing the batch consistency of a first rAAV batch and a second rAAV batch having a predetermined number of viral genomes (VG), the method comprising:
   producing a first rAAV batch in the packaging cell line of any one of embodiments 1 to 26, the production cell line of any one of embodiments 29 to 55 and 58 to 74, or the cell culture composition of any one of embodiments 83 to 88, or the bioreactor of any one of embodiments 89 to 94, or using the production method of any one of embodiments 27, 28, 56, 57, and 103 to 105; and
   producing a second rAAV batch in the packaging cell line of any one of embodiments 1 to 26, the production cell line of any one of embodiments 29 to 55 and 58 to 74, or the cell culture composition of any one of embodiments 83 to 88, or the bioreactor of any one of embodiments 89 to 94, or using the production method of any one of embodiments 27, 28, 56, 57, and 103 to 105;
      wherein a number of viral particles (VP) in the first rAAV batch has the predetermined number of viral genomes (VG) that varies by no more than 50%, 40%, 30%, or 20% compared to a number of viral particles (VP) and a number of viral genomes in the second batch rAAV batch.
[133] A method for increasing the batch consistency of a first rAAV batch and a second rAAV batch, the method comprising:
   producing a first rAAV batch in the packaging cell line of any one of embodiments 1 to 26, the production cell line of any one of embodiments 29 to 55 and 58 to 74, or the cell culture composition of any one of embodiments 83 to 88, or the bioreactor of any one of embodiments 89 to 94, or using the production method of any one of embodiments 27, 28, 56, 57, and 103 to 105; and
   producing a second rAAV batch in the packaging cell line of any one of embodiments 1 to 26, the production cell line of any one of embodiments 29 to 55 and 58 to 74, or the cell culture composition of any one of embodiments 83 to 88, or the bioreactor of any one of embodiments 89 to 94, or using the production method of any one of embodiments 27, 28, 56, 57, and 103 to 105;
      wherein the second rAAV batch has a number of viral genomes that varies by no more than 50%, 40%, 30%, 20%, 10%, or 5% compared to the number of viral genomes in the first rAAV batch.
[134] The method of embodiment 133, wherein the second rAAV batch has a number of viral genomes that varies by no more than 50%, 40%, 30%, 20%, 10%, or 5% prepurification compared to the number of viral genomes in the first rAAV batch.
[135] The method of embodiment 133 or 134, wherein the second rAAV batch has a number of viral particles that varies by no more than 50%, 40%, 30%, 20%, 10%, or 5% compared to the number of viral particles in the first rAAV batch.
[136] The method of any one of embodiments 133 to 135, wherein the second rAAV batch has a number of viral particles that varies by no more than 50%, 40%, 30%, 20%, 10%, or 5% prepurification compared to the number of viral particles in the first rAAV batch.
[137] The method of any one of embodiments 133 to 136, wherein the first rAAV batch and the second rAAV batch are produced from monoclonal cells.
[138] The method of any one of embodiments 133 to 137, wherein the first rAAV batch and the second rAAV batch are produced from cells from different monoclonal cell lines.
[139] The method of any one of embodiments 128 to 138, further comprising the subsequent step of purifying the rAAV virions from cell culture media and/or cell lysate.
[140] A product of rAAV made by the method of any one of embodiments 27, 28, 56, 57, and 103 to 105.

### 6.17. Numbered Embodiments #3

[1] A polynucleotide construct coding for:
   a) one or more helper proteins;
   b) a self-excising element upstream of the one or more helper proteins; and
   c) an inducible promoter upstream of the self-excising element.
[2] The polynucleotide construct of embodiment 1, wherein the self-excising element is operably linked to the inducible promoter.
[3] The polynucleotide construct of embodiment 2, wherein expression of the self-excising element is driven by the inducible promoter.
[4] The polynucleotide construct of any one of embodiments 2-3, wherein the inducible promoter is a tetracycline-responsive promoter element (TRE).
[5] The polynucleotide construct of embodiment 4, wherein the TRE comprises Tet operator (tetO) sequence concatemers fused to a minimal promoter.
[6] The polynucleotide construct of embodiment 5, wherein the minimal promoter is a human cytomegalovirus promoter.
[7] The polynucleotide construct of any one of embodiments 2-6, wherein the inducible promoter comprises a sequence having at least 70%, 80%, 90%, 95%, 99%, or 100% sequence identity to SEQ ID NO: 22.
[8] The polynucleotide construct of any one of embodiments 2-7, wherein transcription is activated from the inducible promoter upon binding of an activator.
[9] The polynucleotide construct of embodiment 8, wherein the activator binds to the inducible promoter in the presence of a first triggering agent.
[10] The polynucleotide construct of any one of embodiments 8-9, further comprising an activator.
[11] The polynucleotide construct of any one of embodiments 8-10, wherein the activator is operably linked to a constitutive promoter.
[12] The polynucleotide construct of embodiment 11, wherein the constitutive promoter is Elalpha promoter or human cytomegalovirus promoter.
[13] The polynucleotide construct of embodiment 12, wherein the E1 alpha promoter comprises at least one mutation.
[14] The polynucleotide construct of any one of embodiments 11-13, wherein the constitutive promoter comprises a sequence having at least 70%, 80%, 90%, 95%, 99%, or 100% sequence identity with SEQ ID NO: 20.
[15] The polynucleotide construct of any one of embodiments 8-14, wherein the activator is reverse tetracycline-controlled transactivator (rTA) comprising a Tet Repressor binding protein (TetR) fused to a VP16 transactivation domain.
[16] The polynucleotide construct of embodiment 15, wherein the rTA comprises four mutations in the tetR DNA binding moiety.
[17] The polynucleotide construct of any one of embodiments 15-16, wherein the rTA comprises a sequence having at least 70%, 80%, 90%, 95%, 99%, or 100% sequence identity to SEQ ID NO: 21.
[18] The polynucleotide construct of any one of embodiments 2-7, wherein the inducible promoter is bound by a repressor in the absence of a first triggering agent.
[19] The polynucleotide construct of any one of embodiments 2-19, wherein the inducible promoter is activated in the presence of a first triggering agent.
[20] The polynucleotide construct of any one of embodiments 18-19, wherein the first triggering agent binds to the repressor.
[21] The polynucleotide construct of any one of embodiments 18-20, wherein the repressor is a tetracycline-controlled transactivator.
[22] The polynucleotide construct of any one of embodiments 18-21, further comprising the repressor.
[23] The polynucleotide construct of any one of embodiments 18-22, wherein the repressor is operably linked to a constitutive promoter.
[24] The polynucleotide construct of any one of embodiments 18-23, further comprising a tetracycline-controlled transactivator.
[25] The polynucleotide construct of any one of embodiments 18-24, wherein the tetracycline-controlled transactivator is operably linked to a constitutive promoter.
[26] The polynucleotide construct of any one of embodiments 18-25, wherein the constitutive promoter is Elalpha promoter.
[27] The polynucleotide construct of any one of embodiments 18-26, wherein the E1 alpha promoter comprises at least one mutation.
[28] The polynucleotide construct of embodiment 27, wherein the constitutive promoter comprises a sequence having at least 70%, 80%, 90%, 95%, 99%, or 100% sequence identity with SEQ ID NO: 20.
[29] The polynucleotide construct of any one of embodiments 21 or 24-28, wherein the tetracycline-controlled transactivator is unbound in the presence of a first triggering agent.
[30] The polynucleotide construct of any one of embodiments 21 or 24-29, wherein the tetracycline-controlled transactivator does not bind to the inducible promoter in the presence of a first triggering agent.
[31] The polynucleotide construct of any one of embodiments 18-30, wherein the constitutive promoter is Elalpha promoter.
[32] The polynucleotide construct of embodiment 31, wherein the E1 alpha promoter comprises at least one mutation.
[33] The polynucleotide construct of any one of embodiments 31-32, wherein the constitutive promoter comprises a sequence having at least 70%, 80%, 90%, 95%, 99%, or 100% sequence identity with SEQ ID NO: 20.
[34] The polynucleotide construct of any one of embodiments 18-33, wherein transcription is activated from the inducible promoter upon binding of the first triggering agent to the repressor.
[35] The polynucleotide construct of any one of embodiments 18-34, wherein the repressor binds to the first triggering agent.
[36] The polynucleotide construct of any one of embodiments 18-35, wherein the first triggering agent is a tetracycline.
[37] The polynucleotide construct of embodiments 36, wherein the tetracycline is doxycycline.
[38] The polynucleotide construct of any one of embodiments 2-3, wherein the inducible promoter is a cumate operator sequence.
[39] The polynucleotide construct of embodiment 38, wherein the cumate operator sequence is downstream of a constitutive promoter.
[40] The polynucleotide construct of embodiment 39, wherein the constitutive promoter is a human cytomegalovirus promoter.
[41] The polynucleotide construct of any one of embodiments 38-40, wherein the inducible promoter is bound by a cymR repressor in the absence of a first triggering agent.
[42] The polynucleotide construct of any one of embodiments 38-41, wherein the inducible promoter is activated in the presence of a first triggering agent.
[43] The polynucleotide construct of embodiment 41, wherein the first triggering agent binds to the cymR repressor.
[44] The polynucleotide construct of any one of embodiments 41 or 43, further comprising a cymR repressor.
[45] The polynucleotide construct of any one of embodiments 41 or 43-44, wherein the cymR repressor is operably linked to a constitutive promoter.
[46] The polynucleotide construct of embodiment 45, wherein the constitutive promoter is E1alpha promoter.
[47] The polynucleotide construct of any one of embodiments 46, wherein the E1 alpha promoter comprises at least one mutation.
[48] The polynucleotide construct of any one of embodiments 46-47, wherein the constitutive promoter comprises a sequence having at least 70%, 80%, 90%, 95%, 99%, or 100% sequence identity with SEQ ID NO: 20.
[49] The polynucleotide construct of any one of embodiments 41-49, wherein the first triggering agent is a cumate.
[50] The polynucleotide construct of any one embodiments 1-49, wherein a sequence coding for the self-excising element comprises a poly A sequence.
[51] The polynucleotide construct of any one embodiments 1-50, wherein the self-excising element is a recombinase.
[52] The polynucleotide construct of embodiment 51, wherein the recombinase is a site-specific recombinase.
[53] The polynucleotide construct of embodiment 51, wherein the recombinase is fused to a ligand binding domain.
[54] The polynucleotide construct of embodiment 51, wherein the recombinase is Cre polypeptide or flippase polypeptide.
[55] The polynucleotide construct of embodiment 54, wherein the Cre polypeptide is fused to a ligand binding domain.
[56] The polynucleotide construct of embodiment 55, wherein the ligand binding domain is a hormone receptor.
[57] The polynucleotide construct of embodiment 56, wherein the hormone receptor is an estrogen receptor.
[58] The polynucleotide construct of embodiment 57, wherein the estrogen receptor comprises a point mutation.
[59] The polynucleotide construct of embodiment 58, wherein the estrogen receptor is ERT2.
[60] The polynucleotide construct of embodiment 51, wherein the recombinase is a Cre-ERT2 polypeptide.
[61] The polynucleotide construct of any one of embodiments 1-61, wherein the self-excising element translocates to the nucleus in the presence of a second triggering agent.
[62] The polynucleotide construct of embodiment 61, wherein the second triggering agent is an estrogen receptor ligand.
[63] The polynucleotide construct of any one of the preceding embodiments, wherein the second triggering agent is a selective estrogen receptor modulator (SERM).
[64] The polynucleotide construct of any one of the preceding embodiments, wherein the second triggering agent is tamoxifen.
[65] The polynucleotide construct of any one of the preceding embodiments, wherein the recombinase is flanked by recombination sites
[66] The polynucleotide construct of any one of the preceding embodiments, wherein the recombination sites are lox sites or flippase recognition target (FRT) sites.
[67] The polynucleotide construct of any one of the preceding embodiments, wherein the lox sites are loxP sites.
[68] The polynucleotide construct of any one of the preceding embodiments, wherein the one or more adenoviral helper proteins comprise E2A and E4.
[69] The polynucleotide construct of any one of the preceding embodiments, wherein the one or more adenoviral helper proteins further comprises a protein tag.
[70] The polynucleotide construct of any one of the preceding embodiments, wherein the protein tag is a FLAG-tag.
[71] The polynucleotide construct of any one of the preceding embodiments, wherein the E2A is FLAG-tagged E2A.
[72] The polynucleotide construct of any one of the preceding embodiments, wherein the sequence coding for E2 and the sequence coding for E4 are separated by an internal ribosome entry site (IRES) or by P2A.
[73] The polynucleotide construct of any one of the preceding embodiments, further comprising a sequence coding for a selectable marker.
[74] The polynucleotide construct of any one of the preceding embodiments, wherein the selectable marker is an antibiotic resistance protein.
[75] The polynucleotide construct of any one of the preceding embodiments, wherein the selectable marker is a split intein linked to an N-terminus of the antibiotic resistance protein or split intein linked to a C-terminus of the antibiotic resistance protein.
[76] The polynucleotide construct of any one of the preceding embodiments, wherein the selectable marker is a leucine zipper linked to an N-terminus of the antibiotic resistance protein or leucine zipper linked to a C-terminus of the antibiotic resistance protein.
[77] The polynucleotide construct of any one of the preceding embodiments, wherein the antibiotic resistance protein is for puromycin resistance or blasticidin resistance.
[78] The polynucleotide construct of any one of the preceding embodiments, further comprising a sequence coding for VA RNA.
[79] The polynucleotide construct of any one of the preceding embodiments, the sequence coding for VA RNA is a transcriptionally dead sequence.
[80] The polynucleotide construct of any one of the preceding embodiments, the sequence coding for VA RNA comprises at least two mutations in the internal promoter.
[81] The polynucleotide construct of any one of the preceding embodiments, wherein expression of VA RNA is driven by a U6 promoter.
[82] The polynucleotide construct of any one of the preceding embodiments, comprising upstream of the sequence coding for VA RNA gene sequence, from 5′ to 3′:
   a) a first part of a U6 promoter sequence;
   b) a first recombination site;
   c) a stuffer sequence;
   d) a second recombination site;
   e) a second part of a U6 promoter sequence.
[83] The polynucleotide construct of any one of the preceding embodiments, wherein the stuffer sequence is excisable by the recombinase.
[84] The polynucleotide construct of any one of the preceding embodiments, wherein the stuffer sequence comprises a sequence encoding a gene.
[85] The polynucleotide construct of any one of the preceding embodiments, wherein the stuffer sequence comprises a promoter.
[86] The polynucleotide construct of any one of the preceding embodiments, wherein the promoter is a constitutive promoter.
[87] The polynucleotide construct of any one of the preceding embodiments, wherein the promoter is a CMV promoter.
[88] The polynucleotide construct of any one of the preceding embodiments, wherein the gene encodes a detectable marker or a selectable marker.
[89] The polynucleotide construct of any one of the preceding embodiments, wherein the selectable marker is an antibiotic resistance protein.
[90] The polynucleotide construct of any one of the preceding embodiments, wherein the selectable marker is a split intein linked to an N-terminus of the antibiotic resistance protein or split intein linked to a C-terminus of the antibiotic resistance protein.
[91] The polynucleotide construct of any one of the preceding embodiments, wherein the selectable marker is a leucine zipper linked to an N-terminus of the antibiotic resistance protein or leucine zipper linked to a C-terminus of the antibiotic resistance protein.
[92] The polynucleotide construct of any one of the preceding embodiments, wherein the selectable marker is a mammalian cell selection element.
[93] The polynucleotide construct of any one of the preceding embodiments, wherein the selectable marker is an auxotrophic selection element.
[94] The polynucleotide construct of any one of the preceding embodiments, wherein the auxotrophic selection element codes for an active protein.
[95] The polynucleotide construct of any one of the preceding embodiments, wherein the active protein is DHFR.
[96] The polynucleotide construct of any one of the preceding embodiments, wherein the auxotrophic selection coding sequence encodes an inactive protein that requires expression of a second auxotrophic selection coding sequence for activity.
[97] The polynucleotide construct of any one of the preceding embodiments, wherein the second auxotrophic selection coding sequence encodes for DHFR Z-Cter or DHFR Z-Nter.
[98] The polynucleotide construct of any one of the preceding embodiments, wherein the inactive protein comprises a DHFR Z-Nter or DHFR Z-Cter
[99] The polynucleotide construct of any one of the preceding embodiments, wherein the selectable marker is DHFR Z-Nter or DHFR Z-Cter.
[100] The polynucleotide construct of any one of the preceding embodiments, wherein the DHFR Z-Nter comprises a sequence having at least 70%, 80%, 90%, 95%, 99%, or 100% sequence identity to SEQ ID NO: 4.
[101] The polynucleotide construct of any one of the preceding embodiments wherein the DHFR Z-Cter comprises a sequence having at least 70%, 80%, 90%, 95%, 99%, or 100% sequence identity to SEQ ID NO: 5.
[102] The polynucleotide construct of any one of the preceding embodiments, wherein the antibiotic resistance protein is for puromycin resistance or blasticidin resistance.
[103] The polynucleotide construct of any one of the preceding embodiments, wherein the detectable marker comprises a luminescent marker or a fluorescent marker.

The polynucleotide construct of any one of the preceding embodiments, wherein the fluorescent marker is GFP, EGFP, RFP, CFP, BFP, YFP, or mCherry.
[105] The polynucleotide construct of any one of the preceding embodiments, wherein the first recombination site is a first lox sequence and the second recombination site is a second lox sequence.
[106] The polynucleotide construct of any one of the preceding embodiments, wherein the first lox sequence is a first loxP site and the second lox sequence is a second loxP site.
[107] The polynucleotide construct of any one of the preceding embodiments, wherein the first recombination site is a first FRT site and the second recombination site is a second FRT site.
[108] The polynucleotide construct of any one of embodiments 1-107 in a vector.
[109] The polynucleotide construct of any one of embodiments 1-107 in a plasmid.
[110] The polynucleotide construct of any one of embodiments 1-107 in a bacterial artificial chromosome or yeast artificial chromosome.
[111] The polynucleotide construct of any one of embodiments 1-110, wherein the polynucleotide construct is a synthetic nucleic acid construct.
[112] The polynucleotide construct of any one of embodiments 1-111 comprising a sequence having at least 70%, 80%, 90%, 95%, 99%, or 100% sequence identity to any one of SEQ ID NO: 9-SEQ ID NO: 19, SEQ ID 23-SEQ ID NO: 32, or SEQ ID NO: 35.
[113] A cell comprising the polynucleotide construct of any one of embodiments 1-112.
[114] The cell of embodiment 113, wherein the polynucleotide is stably integrated into the genome of the cell.
[115] The cell of any one of embodiments 113-114, wherein the cell is a mammalian cell or insect cell.
[116] The cell of any one of embodiments 113-114, wherein the cell is a HEK293 cell, HeLa cell, CHO cell, or SF9 cell.
[117] The cell of any one of embodiments 113-116, wherein the cell expresses E1A protein and E1B protein.
[118] The cell of any one of embodiments 113-117, wherein the cell is DHFR null.
[119] A polynucleotide construct comprising:
   a) a sequence of a first part of a Rep gene;
   b) a sequence of a second part of the Rep gene;
   c) a sequence of a Cap gene; and
   d) an excisable element positioned between the first part of the sequence of Rep gene and the second part of the sequence of the Rep gene.
[120] The polynucleotide construct of any one of the preceding embodiments, wherein the excisable element comprises a stop signaling sequence.
[121] The polynucleotide construct of any one of the preceding embodiments, wherein the excisable element comprises a rabbit beta globin intron.
[122] The polynucleotide construct of any one of the preceding embodiments, wherein the excisable element comprises an exon.
[123] The polynucleotide construct of any one of the preceding embodiments, wherein the excisable element comprises an intron and an exon.
[124] The polynucleotide construct of any one of the preceding embodiments, wherein the excisable element comprises an intron.
[125] The polynucleotide construct of any one of the preceding embodiments, wherein two splice sites are positioned between the sequence of the first part of the Rep gene and the sequence of the second part of the Rep gene.
[126] The polynucleotide construct of any one of the preceding embodiments, wherein the two splice sites are a 5′ splice site and a 3′ splice site.
[127] The polynucleotide construct of any one of the preceding embodiments, wherein the 5′ splice site is a rabbit beta globin 5′ splice site.
[128] The polynucleotide construct of any one of the preceding embodiments, wherein the 3′ splice site is a rabbit beta globin 3′ splice site.
[129] The polynucleotide construct of any one of the preceding embodiments, wherein three splice sites are positioned between the sequence of the first part of the Rep gene and the sequence of the second part of the Rep gene.
[130] The polynucleotide construct of any one of the preceding embodiments, wherein the three splice sites are a 5′ splice site, a first 3′ splice site, and a second 3′ splice site.
[131] The polynucleotide construct of any one of the preceding embodiments, wherein a first 3′ splice site is a duplicate of the second 3′ splice site.
[132] The polynucleotide construct of any one of the preceding embodiments, wherein the first 3′ splice site is a rabbit beta globin 3′ splice site.
[133] The polynucleotide construct of any one of the preceding embodiments, wherein the second 3′ splice site is a rabbit beta globin 3′ splice site.
[134] The polynucleotide construct of any one of the preceding embodiments, wherein the excisable element comprises a recombination site.
[135] The polynucleotide construct of any one of the preceding embodiments, wherein the recombination site is a lox site or FRT site.
[136] The polynucleotide construct of any one of the preceding embodiments, wherein the lox site is a loxP site.
[137] The polynucleotide construct of any one of the preceding embodiments, wherein the excisable element comprises from 5′ to 3′:
   a) the 5′ splice site;
   b) a first recombination site;
   c) the first 3′ splice site;
   d) a stop signaling sequence;
   e) a second recombination site; and
   f) the second 3′ splice site.
[138] The polynucleotide construct of any one of the preceding embodiments, wherein the excisable element comprises from 5′ to 3′:
   a) the 5′ splice site;
   b) a first spacer segment;
   c) a second spacer segment comprising:
      i) a first recombination site;
      ii) the first 3′ splice site;
      iv) a stop signaling sequence; and
      v) a second recombination site; and
   d) a third spacer segment comprising the second 3′ splice site.
[139] The polynucleotide construct of any one of the preceding embodiments, wherein the first spacer sequence comprises an intron.
[140] The polynucleotide construct of any one of the preceding embodiments, wherein the first spacer segment comprises a sequence having at least 70%, 80%, 90%, 95%, 99%, or 100% sequence identity to SEQ ID NO: 1.
[141] The polynucleotide construct of any one of the preceding embodiments, wherein the second spacer segment comprises a sequence having at least 70%, 80%, 90%, 95%, 99%, or 100% sequence identity to SEQ ID NO: 2.
[142] The polynucleotide construct of any one of the preceding embodiments, wherein the third spacer segment comprises a sequence having at least 70%, 80%, 90%, 95%, 99%, or 100% sequence identity to SEQ ID NO: 3.
[143] The polynucleotide construct of any one of the preceding embodiments, wherein the third spacer segment comprises an intron.
[144] The polynucleotide construct of any one of the preceding embodiments, wherein the first spacer segment and the third spacer segment are capable of being excised by endogenous cellular machinery.
[145] The polynucleotide construct of any one of the preceding embodiments, wherein the second spacer segment comprises an exon.
[146] The polynucleotide construct of any one of the preceding embodiments, wherein the second spacer segment further comprises a polyA sequence.
[147] The polynucleotide construct of any one of the preceding embodiments, wherein the polyA sequence is 3′ of the exon.
[148] The polynucleotide construct of any one of the preceding embodiments, wherein the polyA sequence comprises a rabbit beta globin (RBG) polyA sequence.
[149] The polynucleotide construct of any one of embodiments, wherein the second spacer segment comprises from 5′ to 3′:
   a) a first recombination site;
   b) the first 3′ splice site;
   c) an exon;
   d) a stop signaling sequence; and
   e) a second recombination site.
[150] The polynucleotide construct of any one of the preceding embodiments, wherein the first recombination site is a first lox sequence and the second recombination site is a second lox sequence.
[151] The polynucleotide construct of any one of the preceding embodiments, wherein the first lox sequence is a first loxP sequence and a second lox sequence is a second loxP sequence.
[152] The polynucleotide construct of any one of the preceding embodiments, wherein the first recombination site is a first FRT site and the second recombination site is a second FRT site.
[153] The polynucleotide construct of any one of the preceding embodiments, wherein the stop signaling sequence is a termination codon of the exon or a polyA sequence.
[154] The polynucleotide construct of any one of the preceding embodiments, wherein the polyA sequence comprises a rabbit beta globin (RBG) polyA sequence.
[155] The polynucleotide construct of any one of the preceding embodiments, wherein the exon encodes a detectable marker or a selectable marker.
[156] The polynucleotide construct of any one of the preceding embodiments, wherein the detectable marker comprises a luminescent marker or a fluorescent marker.
[157] The polynucleotide construct of any one of the preceding embodiments, wherein the fluorescent marker is GFP, EGFP, RFP, CFP, BFP, YFP, or mCherry.
[158] The polynucleotide construct of any one of the preceding embodiments, wherein the second spacer segment is excisable by a recombinase.
[159] The polynucleotide construct of any one of the preceding embodiments, wherein the recombinase is a site-specific recombinase.
[160] The polynucleotide construct of any one of embodiments, wherein the recombinase is a Cre polypeptide or a Flippase polypeptide.
[161] The polynucleotide construct of any one the preceding embodiments, wherein the Cre polypeptide is fused to a ligand binding domain.
[162] The polynucleotide construct of any one of the preceding embodiments, wherein the ligand binding domain is a hormone receptor.
[163] The polynucleotide construct of any one of the preceding embodiments, wherein the hormone receptor is an estrogen receptor.
[164] The polynucleotide construct of any one of the preceding embodiments, wherein the estrogen receptor comprises a point mutation.
[165] The polynucleotide construct of any one of the preceding embodiments, wherein the estrogen receptor is ERT2.
[166] The polynucleotide construct of any one the preceding embodiments, wherein the recombinase is a Cre-ERT2 polypeptide.
[167] The polynucleotide construct of the preceding embodiments, wherein the recombinase is encoded by a second polynucleotide construct or exogenously provided.
[168] The polynucleotide construct of any one of the preceding embodiments, wherein the Rep gene codes for Rep polypeptides.
[169] The polynucleotide construct of any one of the preceding embodiments, wherein the Cap gene codes for Cap polypeptides.
[170] The polynucleotide construct of any one of the preceding embodiments, wherein transcription of the Rep gene and the Cap gene are driven by native promoters.
[171] The polynucleotide construct of any one of the preceding embodiments, wherein the native promoters comprise P5, P19, and P40.
[172] The polynucleotide construct of any one of the preceding embodiments, wherein the Rep polypeptides are wildtype Rep polypeptides.
[173] The polynucleotide construct of any one of the preceding embodiments, wherein the Rep polypeptides comprise Rep78, Rep68, Rep52, and Rep40.
[174] The polynucleotide construct of any one of the preceding embodiments, wherein a truncated replication associated protein comprising a polypeptide expressed from the sequence of first part of a Rep gene and the exon is capable of being expressed in the absence of the recombinase.
[175] The polynucleotide construct of any one of the preceding embodiments, wherein the Cap polypeptides are wildtype Cap polypeptides.
[176] The polynucleotide construct of any one of the preceding embodiments, wherein the Cap polypeptides are AAV capsid proteins.
[177] The polynucleotide construct of any one of the preceding embodiments, wherein the AAV capsid proteins comprise VP1, VP2, and VP3.
[178] The polynucleotide construct of any one of the preceding embodiments, wherein a serotype of the AAV capsid proteins is selected from the group consisting of AAV1, AAV2, AAV3, AAV4, AAV5, AAV6, AAV7, AAV8, AAV9, AAV 10, AAV11, AAV 12, AAV13, AAV 14, AAV 15 and AAV 16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, and AAVhu68.
[179] The polynucleotide construct of any one of the preceding embodiments, further comprising a sequence coding for a selectable marker.
[180] The polynucleotide construct of any one of the preceding embodiments, wherein the selectable marker is a mammalian cell selection element.
[181] The polynucleotide construct of any one of the preceding embodiments, wherein the selectable marker is an auxotrophic selection element.
[182] The polynucleotide construct of any one of the preceding embodiments, wherein the auxotrophic selection element codes for an active protein.
[183] The polynucleotide construct of any one of the preceding embodiments, wherein the active protein is DHFR.
[184] The polynucleotide construct of any one of the preceding embodiments, wherein the auxotrophic selection coding sequence encodes an inactive protein that requires expression of a second auxotrophic selection coding sequence for activity.
[185] The polynucleotide construct of any one of the preceding embodiments, wherein the second auxotrophic selection coding sequence encodes for DHFR Z-Cter or DHFR Z-Nter.
[186] The polynucleotide construct of any one of the preceding embodiments, wherein the inactive protein comprises a DHFR Z-Nter or DHFR Z-Cter
[187] The polynucleotide construct of any one of the preceding embodiments, wherein the selectable marker is DHFR Z-Nter or DHFR Z-Cter.
[188] The polynucleotide construct of any one of embodiments 185-187, wherein the DHFR Z-Nter comprises a sequence having at least 70%, 80%, 90%, 95%, 99%, or 100% sequence identity to SEQ ID NO: 4.
[189] The polynucleotide construct of any one of embodiments 185-188, wherein the DHFR Z-Cter comprises a sequence having at least 70%, 80%, 90%, 95%, 99%, or 100% sequence identity to SEQ ID NO: 5.
[190] The polynucleotide construct of any one of the preceding embodiments, wherein the selectable marker is an antibiotic resistance protein.
[191] The polynucleotide construct of any one of the preceding embodiments, wherein the selectable marker is a split intein linked to an N-terminus of the antibiotic resistance protein or split intein linked to a C-terminus of the antibiotic resistance protein.
[192] The polynucleotide construct of any one of the preceding embodiments, wherein the selectable marker is a leucine zipper linked to an N-terminus of the antibiotic resistance protein or leucine zipper linked to a C-terminus of the antibiotic resistance protein.
[193] The polynucleotide construct of any one of the preceding embodiments, wherein the antibiotic resistance protein is for puromycin resistance or blasticidin resistance.
[194] The polynucleotide construct of any one of embodiments 119-193 further comprising the polynucleotide construct of any one of embodiments 1-112.
[195] The polynucleotide construct of any one of embodiments 119-194 in a vector.
[196] The polynucleotide construct of any one of embodiments 119-194 in a plasmid.
[197] The polynucleotide construct of any one of embodiments 119-194 in a bacterial artificial chromosome or yeast artificial chromosome.
[198] The polynucleotide construct of any one of embodiments 119-197, wherein the polynucleotide construct is a synthetic nucleic acid construct.
[199] The polynucleotide construct of any one of embodiments 119-198 comprising a sequence having at least 70%, 80%, 90%, 95%, 99%, or 100% sequence identity to any one of SEQ ID NO: 1-SEQ ID NO: 3, SEQ ID 6-SEQ ID NO: 8, or SEQ ID NO: 32.
[200] The polynucleotide construct of any one of embodiments 119-199, further comprising a sequence coding for VA RNA.
[201] The polynucleotide construct of embodiment 200, the sequence coding for VA RNA is a transcriptionally dead sequence.
[202] The polynucleotide construct of any one of the preceding embodiments, the sequence coding for VA RNA comprises at least two mutations in the internal promoter.
[203] The polynucleotide construct of any one of the preceding embodiments, wherein expression of VA RNA is driven by a U6 promoter.
[204] The polynucleotide construct of any one of the preceding embodiments, comprising upstream of the sequence coding for VA RNA gene sequence, from 5′ to 3′:
   a) a first part of a U6 promoter sequence;
   b) a first recombination site;
   c) a stuffer sequence;
   d) a second recombination site;
   e) a second part of a U6 promoter sequence.
[205] The polynucleotide construct of any one of the preceding embodiments, wherein the stuffer sequence is excisable by the recombinase.
[206] The polynucleotide construct of any one of the preceding embodiments, wherein the stuffer sequence comprises a sequence encoding a gene.
[207] The polynucleotide construct of any one of the preceding embodiments, wherein the stuffer sequence comprises a promoter.
[208] The polynucleotide construct of any one of the preceding embodiments, wherein the promoter is a constitutive promoter.
[209] The polynucleotide construct of any one of the preceding embodiments, wherein the promoter is a CMV promoter.
[210] A cell comprising the polynucleotide construct of any one of embodiments 119-209.
[211] The cell of embodiment 210, wherein the polynucleotide is stably integrated into the genome of the cell.
[212] The cell of any one of embodiments 210-211, wherein the cell is a mammalian cell or insect cell.
[213] The cell of any one of embodiments 210-211, wherein the cell is a HEK293 cell, HeLa cell, CHO cell, or SF9 cell.
[214] The cell of any one of embodiments 210-213, wherein the cell expresses E1A protein and E1B protein.
[215] The cell of any one of embodiments 210-214, wherein the cell is DHFR null.
[216] A polynucleotide construct coding for a VA RNA, wherein a sequence coding for the VA RNA comprises at least two mutations in an internal promoter.
[217] The polynucleotide construct of any one of the preceding embodiments, wherein the sequence coding for the VA RNA comprises a sequence coding for a transcriptionally dead VA RNA.
[218] The polynucleotide construct of any one of the preceding embodiments, wherein the sequence coding for the VA RNA comprises a deletion of from about 5-10 nucleotides in the promoter region.
[219] The polynucleotide construct of any one of the preceding embodiments, wherein the sequence coding for the VA RNA comprises at least one mutation.
[220] The polynucleotide construct of any one of the preceding embodiments, wherein the at least one mutation is in the A Box promoter region.
[221] The polynucleotide construct of any one of the preceding embodiments, wherein the at least one mutation is in the B Box promoter region.
[222] The polynucleotide construct of any one of the preceding embodiments, wherein the at least one mutation is G16A and G60A.
[223] The polynucleotide construct of any one of the preceding embodiments, wherein expression of the VA RNA is driven by a U6 promoter.
[224] The polynucleotide construct of any one of the preceding embodiments, comprising upstream of the VA RNA gene sequence, from 5′ to 3′:
   a) a first part of a U6 promoter sequence;
   b) a first recombination site;
   c) a stuffer sequence;
   d) a second recombination site;
   e) a second part of a U6 promoter sequence.
[225] The polynucleotide construct of any one of the preceding embodiments, wherein the stuffer sequence is excisable by a recombinase.
[226] The polynucleotide construct of any one of the preceding embodiments, wherein the stuffer sequence comprises a sequence encoding a gene.
[227] The polynucleotide construct of any one of the preceding embodiments, wherein the stuffer sequence comprises a promoter.
[228] The polynucleotide construct of any one of the preceding embodiments, wherein the promoter is a constitutive promoter.
[229] The polynucleotide construct of any one of the preceding embodiments, wherein the promoter is a CMV promoter.
[230] The polynucleotide construct of any one of the preceding embodiments, wherein the gene encodes a detectable marker or a selectable marker.
[231] The polynucleotide construct of any one of the preceding embodiments, wherein the selectable marker is a mammalian cell selection element.
[232] The polynucleotide construct of any one of the preceding embodiments, wherein the selectable marker is an auxotrophic selection element.
[233] The polynucleotide construct of any one of the preceding embodiments, wherein the auxotrophic selection element codes for an active protein.
[234] The polynucleotide construct of any one of the preceding embodiments, wherein the active protein is DHFR.
[235] The polynucleotide construct of any one of the preceding embodiments, wherein the auxotrophic selection coding sequence encodes an inactive protein that requires expression of a second auxotrophic selection coding sequence for activity.
[236] The polynucleotide construct of any one of the preceding embodiments, wherein the second auxotrophic selection coding sequence encodes for DHFR Z-Cter or DHFR Z-Nter.
[237] The polynucleotide construct of embodiment 236, wherein the inactive protein comprises a DHFR Z-Nter or DHFR Z-Cter
[238] The polynucleotide construct of any one of embodiments 236-237, wherein the selectable marker is DHFR Z-Nter or DHFR Z-Cter.
[239] The polynucleotide construct of any one of embodiments 236-238, wherein the DHFR Z-Nter comprises a sequence having at least 70%, 80%, 90%, 95%, 99%, or 100% sequence identity to SEQ ID NO: 4.
[240] The polynucleotide construct of any one of embodiments 236-239, wherein the DHFR Z-Cter comprises a sequence having at least 70%, 80%, 90%, 95%, 99%, or 100% sequence identity to SEQ ID NO: 5.
[241] The polynucleotide construct of any one of the preceding embodiments, wherein the selectable marker is an antibiotic resistance protein.
[242] The polynucleotide construct of any one of the preceding embodiments, wherein the selectable marker is a split intein linked to an N-terminus of the antibiotic resistance protein or split intein linked to a C-terminus of the antibiotic resistance protein.
[243] The polynucleotide construct of any one of the preceding embodiments, wherein the selectable marker is a leucine zipper linked to an N-terminus of the antibiotic resistance protein or leucine zipper linked to a C-terminus of the antibiotic resistance protein.
[244] The polynucleotide construct of any one of the preceding embodiments, wherein the antibiotic resistance protein is for puromycin resistance or blasticidin resistance.
[245] The polynucleotide construct of any one of the preceding embodiments, wherein the detectable marker comprises a luminescent marker or a fluorescent marker.
[246] The polynucleotide construct of any one of the preceding embodiments, wherein the fluorescent marker is GFP, EGFP, RFP, CFP, BFP, YFP, or mCherry.
[247] The polynucleotide construct of any one of the preceding embodiments, further comprising a sequence coding for a recombinase.
[248] The polynucleotide construct of any one of the preceding embodiments, wherein the recombinase is exogenously provided.
[249] The polynucleotide construct of any one of the preceding embodiments, wherein the recombinase is a site-specific recombinase.
[250] The polynucleotide construct of any one of the preceding embodiments, wherein the recombinase is a Cre polypeptide or a Flippase polypeptide.
[251] The polynucleotide construct of any one the preceding embodiments, wherein the Cre polypeptide is fused to a ligand binding domain.
[252] The polynucleotide construct of any one of the preceding embodiments, wherein the ligand binding domain is a hormone receptor.
[253] The polynucleotide construct of any one of the preceding embodiments, wherein the hormone receptor is an estrogen receptor.
[254] The polynucleotide construct of any one of the preceding embodiments, wherein the estrogen receptor comprises a point mutation.
[255] The polynucleotide construct of any one of the preceding embodiments, wherein the estrogen receptor is ERT2.
[256] The polynucleotide construct of any one the preceding embodiments, wherein the recombinase is a Cre-ERT2 polypeptide.
[257] The polynucleotide construct of any one of the preceding embodiments, wherein the first recombination site is a first lox sequence and the second recombination site is a second lox sequence.
[258] The polynucleotide construct of any one of the preceding embodiments, wherein the first lox sequence is a first loxP site and the second lox sequence is a second loxP site.
[259] The polynucleotide construct of any one of the preceding embodiments, wherein the first recombination site is a first FRT site and the second recombination site is a second FRT site.
[260] The polynucleotide construct of any one of the preceding embodiments, further comprising a sequence coding for a selectable marker.
[261] The polynucleotide construct of any one of the preceding embodiments, wherein the selectable marker is an antibiotic resistance protein.
[262] The polynucleotide construct of any one of the preceding embodiments, wherein the selectable marker is a split intein linked to an N-terminus of the antibiotic resistance protein or split intein linked to a C-terminus of the antibiotic resistance protein.
[263] The polynucleotide construct of any one of the preceding embodiments, wherein the selectable marker is a leucine zipper linked to an N-terminus of the antibiotic resistance protein or leucine zipper linked to a C-terminus of the antibiotic resistance protein.
[264] The polynucleotide construct of any one of the preceding embodiments, wherein the antibiotic resistance protein is for puromycin resistance or blasticidin resistance.
[265] The polynucleotide construct of any one of embodiments 216-264 further comprising a sequence of the polynucleotide construct of any one of embodiments 1-112.
[266] The polynucleotide construct of any one of embodiments 216-265 further comprising a sequence of the polynucleotide sequence of any one of embodiments 119-209.
[267] The polynucleotide construct of any one of embodiments 216-266 in a vector.
[268] The polynucleotide construct of any one of embodiments 216-266 in a plasmid.
[269] The polynucleotide construct of any one of embodiments 216-266 in a bacterial artificial chromosome or yeast artificial chromosome.
[270] The polynucleotide construct of any one of embodiments 216-269, wherein the polynucleotide construct is a synthetic nucleic acid construct.
[271] The polynucleotide construct of any one of embodiments 216-270 comprising a sequence having at least 70%, 80%, 90%, 95%, 99%, or 100% sequence identity to any one of SEQ ID NO: 13-SEQ ID NO: 19 or SEQ ID 23-SEQ ID NO: 26.
[272] A cell comprising the polynucleotide construct of any one of embodiments 216-271.
[273] The cell of embodiment 272, wherein the polynucleotide construct is stably integrated into the genome of the cell.
[274] The cell of any one of embodiments 272-273, wherein the cell is a mammalian cell or insect cell.
[275] The cell of any one of embodiments 272-273, wherein the cell is a HEK293 cell, HeLa cell, CHO cell, or SF9 cell.
[276] The cell of any one of embodiments 272-275, wherein the cell expresses E1A protein and E1B protein.
[277] The cell of any one of embodiments 272-276, wherein the cell is DHFR null.
[278] A cell comprising the polynucleotide construct of any one of embodiments 1-112 and the polynucleotide construct of any one of embodiments 119-209.
[279] A cell comprising the polynucleotide construct of any one of embodiments 1-112 and the polynucleotide construct of any one of embodiments 216-271.
[280] A cell comprising the polynucleotide construct of any one of embodiments Z and the polynucleotide construct of any one of embodiments 119-209.
[281] A cell comprising the polynucleotide construct of any one of embodiments 1-112, the polynucleotide sequence of any one of embodiments 119-209, and the polynucleotide construct of any one of embodiments 216-271.
[282] The cell of any one of embodiments 278-281, wherein the polynucleotide construct of any one of embodiments 1-112 is stably integrated into the genome of the cell.
[283] The cell of any one of embodiments 278-282, wherein the polynucleotide construct of any one of embodiments 119-209 is stably integrated into the genome of the cell.
[284] The cell of any one of embodiments 278-283, wherein the polynucleotide construct of any one of embodiments 216-271 is stably integrated into the genome of the cell.
[285] The cell of any one of embodiments 278-284, wherein the polynucleotide construct of any one of embodiments 1-112 and the polynucleotide construct of any one of embodiments 119-209 are separately stably integrated into the genome of the cell.
[286] The cell of any one of embodiments 278-285, wherein the polynucleotide construct of any one of embodiments 1-112 and the polynucleotide construct of any one of embodiments 216-271 are separately stably integrated into the genome of the cell.
[287] The cell of any one of embodiments 278-286, wherein the polynucleotide construct of any one of embodiments 216-271 and the polynucleotide construct of any one of embodiments 119-209 are separately stably integrated into the genome of the cell.
[288] The cell of any one of embodiments 278-287, wherein a plurality of the polynucleotide construct of any one of embodiments 1-112 are stably integrated into the genome of the cell.
[289] The cell of any one of embodiments 278-288, wherein a plurality of the polynucleotide construct of any one of embodiments 119-209 are stably integrated into the genome of the cell.
[290] The cell of any one of embodiments 278-289, wherein a plurality of the polynucleotide construct of any one of embodiments 216-271 are stably integrated into the genome of the cell.
[291] The cell of any one of embodiments 278-290, wherein a plurality of the polynucleotide construct of any one of embodiments 1-112 and a plurality of the polynucleotide construct of any one of embodiments 119-209 are stably integrated into the genome of the cell.
[292] The cell of any one of embodiments 278-291, wherein a plurality of the polynucleotide construct of any one of embodiments 1-112, a plurality of the polynucleotide construct of any one of embodiments 119-209, and a plurality of the polynucleotide construct of any one of embodiments 216-271 are stably integrated into the genome of the cell.
[293] The cell of any one of embodiments 278-292, wherein a plurality of the polynucleotide construct of any one of embodiments 1-112 are separately stably integrated into the genome of the cell.
[294] The cell of any one of embodiments 278-293, wherein a plurality of the polynucleotide construct of any one of embodiments 119-209 are separately stably integrated into the genome of the cell.
[295] The cell of any one of embodiments 278-294, wherein a plurality of the polynucleotide construct of any one of embodiments 216-271 are separately stably integrated into the genome of the cell.
[296] The cell of any one of embodiments 278-295, wherein a plurality of the polynucleotide construct of any one of embodiments 1-112 and a plurality of the polynucleotide construct of any one of embodiments 119-209 are separately stably integrated into the genome of the cell.
[297] The cell of any one of embodiments 278-296, wherein a plurality of the polynucleotide construct of any one of embodiments 1-112, a plurality of the polynucleotide construct of any one of embodiments 119-209, and a plurality of the polynucleotide construct of any one of embodiments 216-271 are separately stably integrated into the genome of the cell.
[298] The cell of any one of embodiments 278-297, wherein only a single non-auxotrophic selection is required to maintain a stable integration of the polynucleotide construct of any one of embodiments 1-112 and the polynucleotide construct of 119-209 in the genome of the cell.
[299] The cell of any one of embodiments 278-298, wherein only a single non-auxotrophic selection is required to maintain a stable integration of the polynucleotide construct of any one of embodiments 1-112 and the polynucleotide construct of 216-271 in the genome of the cell.
[300] The cell of any one of embodiments 278-299, wherein only a single non-auxotrophic selection is required to maintain a stable integration of the polynucleotide construct of any one of embodiments 216-271 and the polynucleotide construct of 119-209 in the genome of the cell.
[301] The cell of any one of embodiments 278-300, wherein the cell is a mammalian cell or insect cell.
[302] The cell of any one of embodiments 278-301, wherein the cell is a HEK293 cell, HeLa cell, CHO cell, or SF9 cell.
[303] The cell of any one of embodiments 278-302, wherein the cell expresses E1A protein and E1B protein.
[304] The cell of any one of embodiments 278-303, further comprising a payload construct.
[305] The cell of embodiment 304, wherein the payload construct comprises a sequence having at least 70%, 80%, 90%, 95%, 99%, or 100% sequence identity to SEQ ID NO: 33.
[306] The cell of any one of embodiments 304-305, wherein the payload construct comprises a sequence of a payload flanked by ITR sequences.
[307] The cell of any one of the preceding embodiments, wherein expression of the sequence of the payload is driven by a constitutive promoter.
[308] The cell of any one of the preceding embodiments, wherein the constitutive promoter and sequence of the payload are flanked by ITR sequences.
[309] The cell of any one of the preceding embodiments, wherein the sequence of the payload comprises a polynucleotide sequence coding for a gene.
[310] The cell of any one of the preceding embodiments, wherein the gene codes for a selectable marker or detectable marker.
[311] The cell of any one of the preceding embodiments, wherein the gene codes for a therapeutic polypeptide or transgene.
[312] The cell of any one of the preceding embodiments, wherein the sequence of the payload comprises a polynucleotide sequence coding for a therapeutic polynucleotide.
[313] The cell of any one of the preceding embodiments, wherein the therapeutic polynucleotide is a tRNA suppressor or a guide RNA.
[314] The cell of any one of the preceding embodiments, wherein the guide RNA is a polyribonucleotide capable of binding to a protein.
[315] The cell of any one of the preceding embodiments, wherein the protein is nuclease.
[316] The cell of any one of the preceding embodiments, wherein the protein is a Cas protein, an ADAR protein, or an ADAT protein.
[317] The cell of any one of the preceding embodiments, wherein the Cas protein is catalytically inactive Cas protein.
[318] The cell of any one of the preceding embodiments, wherein the payload construct is stably integrated into the genome of the cell.
[319] The cell of any one of the preceding embodiments, wherein a plurality of the payload construct are stably integrated into the genome of the cell.
[320] The cell of any one of the preceding embodiments, wherein the plurality of the payload constructs are separately stably integrated into the genome of the cell.
[321] The cell of any one of the preceding embodiments, wherein the payload construct further comprises a sequence coding for a selectable marker or detectable marker outside of the ITR sequences.
[322] The polynucleotide construct of any one of the preceding embodiments, wherein the selectable marker is a mammalian cell selection element.
[323] The polynucleotide construct of any one of the preceding embodiments, wherein the selectable marker is an auxotrophic selection element.
[324] The polynucleotide construct of any one of the preceding embodiments, wherein the auxotrophic selection element codes for an active protein.
[325] The polynucleotide construct of any one of the preceding embodiments, wherein the active protein is DHFR.
[326] The polynucleotide construct of any one of the preceding embodiments, wherein the auxotrophic selection coding sequence encodes an inactive protein that requires expression of a second auxotrophic selection coding sequence for activity.
[327] The polynucleotide construct of any one of the preceding embodiments, wherein the second auxotrophic selection coding sequence encodes for DHFR Z-Cter or DHFR Z-Nter.
[328] The polynucleotide construct of embodiment 327, wherein the inactive protein comprises a DHFR Z-Nter or DHFR Z-Cter
[329] The polynucleotide construct of any one of embodiments 327-328, wherein the selectable marker is DHFR Z-Nter or DHFR Z-Cter.
[330] The polynucleotide construct of any one of embodiments 327-329, wherein the DHFR Z-Nter comprises a sequence having at least 70%, 80%, 90%, 95%, 99%, or 100% sequence identity to SEQ ID NO: 4.
[331] The polynucleotide construct of any one of embodiments 327-330, wherein the DHFR Z-Cter comprises a sequence having at least 70%, 80%, 90%, 95%, 99%, or 100% sequence identity to SEQ ID NO: 5.
[332] The cell of any one of the preceding embodiments, wherein the selectable marker is an antibiotic resistance protein.
[333] The cell of any one of the preceding embodiments, wherein the selectable marker outside of the ITR sequences is a split intein linked to an N-terminus of the antibiotic resistance protein or split intein linked to a C-terminus of the antibiotic resistance protein.
[334] The cell of any one of the preceding embodiments, wherein the selectable marker outside of the ITR sequences is a leucine zipper linked to an N-terminus of the antibiotic resistance protein or leucine zipper linked to a C-terminus of the antibiotic resistance protein.
[335] The cell of any one of the preceding embodiments, wherein the antibiotic resistance protein is for puromycin resistance or blasticidin resistance.
[336] The cell of any one of the preceding embodiments, wherein only a single non-auxotrophic selection is required to maintain a stable integration of the polynucleotide construct of any one of embodiments 1-112, the polynucleotide construct of 119-209, and the payload construct of any one of the preceding embodiments in the genome of the cell.
[337] The cell of any one of the preceding embodiments, wherein only a single non-auxotrophic selection is required to maintain a stable integration of the polynucleotide construct of any one of embodiments 1-112, the polynucleotide construct of any one of embodiments 216-271, and the payload construct of any one of the preceding embodiments in the genome of the cell.
[338] The cell of any one of the preceding embodiments, wherein only a single non-auxotrophic selection is required to maintain a stable integration of the polynucleotide construct of any one of embodiments 216-271, the polynucleotide construct of 119-209, and the payload construct of any one of the preceding embodiments in the genome of the cell.
[339] The cell of any one of the preceding embodiments, wherein the payload construct is in a plasmid.
[340] The cell of any one of the preceding embodiments, wherein the payload construct is in a bacterial artificial chromosome or yeast artificial chromosome.
[341] The cell of any one of the preceding embodiments, wherein the payload construct is stably integrated into the genome of the cell.
[342] The cell of any one of the preceding embodiments, wherein the payload construct is a synthetic nucleic acid construct.
[343] The cell of any one of the preceding embodiments, wherein the cell is capable of producing an rAAV virion that encapsidates the sequence of the payload.
[344] The cell of any one of the preceding embodiments, wherein the cell is capable of producing an rAAV virion upon addition of at least one triggering agent.
[345] The cell of any one of the preceding embodiments, wherein the rAAV virion comprising the capsid protein and the payload nucleic acid sequence have an infectivity of no less than 50%, 60%, 70%, 80%, 90%, 95%, or 99% at an MOI of 1×10⁵ vg/target cell or less.
[346] The cell of any one of the preceding embodiments, wherein the rAAV virions have an increased infectivity compared rAAV virions produced by an otherwise comparable the population of cells capable of producing rAAV virions upon transient transfection at the same MOI.
[347] The cell of any one of the preceding embodiments, wherein the rAAV virions have at least 1%, 5%, 10%, 15%, 20%, 30%, 40%, or 50% greater infectivity compared rAAV virions produced by an otherwise comparable the population of cells capable of producing rAAV virions upon transient transfection at the same MOI.
[348] The cell of any one of the preceding embodiments, wherein the rAAV virions have at least 50%, 60%, 70%, 80%, 90%, 95%, 97%, 98%, 99%, or 100% infectivity as compared to AAV virions produced by a cell having wildtype AAV at the same MOI.
[349] The cell of any one of the preceding embodiments, wherein the rAAV virions have at least 50%, 60%, 70%, 80%, 90%, 95%, 97%, 98%, 99%, or 100% infectivity as compared to AAV virions at the same MOI.
[350] The cell of any one of the preceding embodiments, wherein the AAV virions are wildtype AAV virions produced by a cell having wildtype AAV.
[351] The cell of any one of the preceding embodiments, wherein the MOI is 1×10¹, 1×10², 2×10³, 5×10⁴, or 1×10⁵ vg/target cell.
[352] The cell of any one of the preceding embodiments, wherein the MOI is selected from a range of 1×10¹ to 1×10⁵ vg/target cell.
[353] The cell of any one of the preceding embodiments, wherein the cell is conditionally capable of producing rAAV virions having an encapsidation ratio of no less than 0.5, 0.6, 0.7, 0.8, 0.9, 0.95, 0.97, or 0.99.
[354] The cell of any one of the preceding embodiments, wherein the rAAV virions have an encapsidation ratio of no less than 0.5, 0.6, 0.7, 0.8, 0.9, 0.95, 0.97, or 0.99 prior to purification.
[355] The cell of any one of the preceding embodiments, wherein the rAAV virions have a concentration of greater than 1×10¹¹ or no less than 5×10¹¹, 1×10¹², 5×10¹², 1×10¹³ or 1×10¹⁴ viral genomes per milliliter prior to purification.
[356] The cell of any one of the preceding embodiments, wherein the cell is capable of producing rAAV virions comprising the payload nucleic acid sequence at a titer of greater than 1×10¹¹ or no less than 5×10¹¹, 1×10¹², 5×10¹², 1×10¹³ or 1×10¹⁴ viral genomes per milliliter.
[357] The cell of any one of the preceding embodiments, wherein the cell is capable of producing rAAV virions comprising the payload nucleic acid sequence at a concentration of greater than 1×10¹¹ or no less than 5×10¹¹, 1×10¹², 5×10¹², 1×10¹³ or 1×10¹⁴ viral genomes per milliliter prior to purification.
[358] A population of cells capable of producing rAAV virions having an encapsidation ratio of no less than 0.5, 0.6, 0.7, 0.8, 0.9, 0.95, 0.97, or 0.99.
[359] A population of cells comprising the polynucleotide construct of any one of embodiments 1-112 and the polynucleotide construct of any one of embodiments 119-209.
[360] A population of cells comprising the polynucleotide construct of any one of embodiments 1-112 and the polynucleotide construct of any one of embodiments 216-271.
[361] A population of cells comprising the polynucleotide construct of any one of embodiments 216-271 and the polynucleotide construct of any one of embodiments 119-209.
[362] A population of cells comprising the polynucleotide construct of any one of embodiments 1-112, the polynucleotide sequence of any one of embodiments 119-209, and the polynucleotide construct of any one of embodiments 216-271.
[363] The population of cells of any one of the preceding embodiments, wherein the polynucleotide construct of any one of embodiments 1-112 is stably integrated into the genome of the population of cells.
[364] The population of cells of any one of the preceding embodiments, wherein the polynucleotide construct of any one of embodiments 119-209 is stably integrated into the genome of the cell.
[365] The population of cells of any one of the preceding embodiments, wherein the polynucleotide construct of any one of embodiments 216-271 is stably integrated into the genome of the cell.
[366] The population of cells of any one of the preceding embodiments, wherein the payload construct of any one of preceding embodiments is stably integrated into the genome of the population of cells.
[367] The population of cells of any one of the preceding embodiments, wherein the population of cells are a plurality of a cell of any one the preceding embodiments.
[368] The population of cells of any one of the preceding embodiments, wherein the population of cells are a population of mammalian cells or a population of insect cells.
[369] The population of cells of any one of the preceding embodiments, wherein the population of cells are a population of HEK293 cells, HeLa cells, CHO cells, or SF9 cells.
[370] The population of cells of any one of the preceding embodiments, wherein the cell expresses E1A protein and E1B protein.
[371] The population of cells of any one of the preceding embodiments, further comprising a payload construct.
[372] The population of cells of any one of the preceding embodiments, wherein the payload construct comprises a sequence of a payload flanked by ITR sequences.
[373] The population of cells of any one of the preceding embodiments, wherein expression of the payload is driven by a constitutive promoter.
[374] The population of cells of any one of the preceding embodiments, wherein the constitutive promoter and sequence of the payload are flanked by ITR sequences.
[375] The population of cells of any one of the preceding embodiments, wherein the payload comprises a polynucleotide sequence encoding a gene.
[376] The population of cells of any one of the preceding embodiments, wherein the gene codes for a selectable marker or detectable marker.
[377] The population of cells of any one of the preceding embodiments, wherein the gene codes for a therapeutic polypeptide or transgene.
[378] The population of cells of any one of the preceding embodiments, wherein the payload comprises a polynucleotide sequence coding for a therapeutic polynucleotide.
[379] The population of cells of any one of the preceding embodiments, wherein the therapeutic polynucleotide is a tRNA suppressor or a guide RNA.
[380] The population of cells of any one of the preceding embodiments, wherein the guide RNA is a polyribonucleotide capable of binding to a protein.
[381] The population of cells of any one of the preceding embodiments, wherein the protein is nuclease.
[382] The population of cells of any one of the preceding embodiments, wherein the protein is a Cas protein, an ADAR protein, or an ADAT protein.
[383] The population of cells of any one of the preceding embodiments, wherein the Cas protein is catalytically inactive Cas protein.
[384] The population of cells of any one of the preceding embodiments, wherein the payload construct is stably integrated into the genome of the cell.
[385] The population of cells of any one of the preceding embodiments, wherein the payload construct further comprises a sequence coding for a selectable marker or detectable marker outside of the ITR sequences.
[386] The population of cells of any one of the preceding embodiments, wherein the selectable marker is an antibiotic resistance protein.
[387] The population of cells of any one of the preceding embodiments, wherein the selectable marker outside of the ITR sequences is a split intein linked to an N-terminus of the antibiotic resistance protein or split intein linked to a C-terminus of the antibiotic resistance protein.
[388] The population of cells of any one of the preceding embodiments, wherein the selectable marker outside of the ITR sequences is a leucine zipper linked to an N-terminus of the antibiotic resistance protein or leucine zipper linked to a C-terminus of the antibiotic resistance protein.
[389] The population of cells of any one of the preceding embodiments, wherein the antibiotic resistance protein is for puromycin resistance or blasticidin resistance.
[390] The population of cells of any one of the preceding embodiments, wherein the payload construct is in a plasmid.
[391] The population of cells of any one of the preceding embodiments, wherein the payload construct is in a bacterial artificial chromosome or yeast artificial chromosome.
[392] The population of cells of any one of the preceding embodiments, wherein the payload construct is stably integrated into the genomes of the population of cells.
[393] A population of cells produced by expanding a cell of any one of the preceding embodiments.
[394] The population of cells of any one of the preceding embodiments, wherein expanding comprises passaging the cell at least three times.
[395] The population of cells of any one the preceding embodiments, wherein a cell of the population of cells is capable of conditionally producing recombinant AAV (rAAV) virions upon addition of at least two triggering agents.
[396] The population of cells of any one the preceding embodiments, wherein the cell is capable of conditionally producing rAAV virions upon addition of at least two triggering agents.
[397] The population of cells of any one the preceding embodiments, wherein the at least two triggering agents comprise doxycycline and tamoxifen.
[398] The population of cells of any one the preceding embodiments, wherein the at least two triggering agents induce the expression and translocation of an excising element to the nucleus.
[399] The population of cells of any one the preceding embodiments, wherein a cell of the population of cells is capable of conditionally producing rAAV virions upon addition of an excising element.
[400] The population of cells of any one the preceding embodiments, wherein the excising element is a recombinase.
[401] The population of cells of any one the preceding embodiments, wherein the excising element is a site-specific recombinase.
[402] The population of cells of any one the preceding embodiments, wherein the excising element is a Cre polypeptide or a flippase polypeptide.
[403] The population of cells of any one of the preceding embodiments, wherein the excising element is hormone regulated.
[404] The population of cells of any one of the preceding embodiments, wherein the population of cells are conditionally capable of producing rAAV virions within which are packaged an expressible polynucleotide encoding a payload; and wherein a population of virions produced by the population of cells are more homogenous than a population of virions produced by an otherwise comparable the population of cells capable of producing rAAV virions upon transient transfection.
[405] The population of cells of any one of the preceding embodiments, wherein the population of virions produced by the population of cells has a ratio of viral genomes to transduction units of about 500:1 to 1:1.
[406] The population of cells of any one of the preceding embodiments, wherein the population of virions produced by the population of cells has a ratio of vector genomes to infectious unit of 100:1.
[407] The population of cells of any one of the preceding embodiments, wherein production of virions is inducible upon addition of a triggering agent.
[408] The population of cells of any one of the preceding embodiments, wherein production of virions is inducible upon addition of at least two triggering agents.
[409] The population of cells of any one the preceding embodiments, wherein the population of cells is conditionally capable of producing rAAV virions having an encapsidation ratio of no less than 0.5, 0.6, 0.7, 0.8, 0.9, 0.95, 0.97, or 0.99.
[410] The population of cells of any one the preceding embodiments, wherein the rAAV virions have an encapsidation ratio of no less than 0.5, 0.6, 0.7, 0.8, 0.9, 0.95, 0.97, or 0.99 prior to purification.
[411] The population of cells of any one the preceding embodiments, wherein the population of cells are capable of reaching a viable cell density of no less than 1×10⁶, 2×10⁶, 5×10⁶, or 1×10⁷ cells per milliliter.
[412] The population of cells of any one the preceding embodiments, wherein the rAAV virions have a concentration of greater than 1×10¹¹ or no less than 5×10¹¹, 1×10¹², 5×10¹², 1×10¹³ or 1×10¹⁴ viral genomes per milliliter prior to purification.
[413] The population of cells of any one the preceding embodiments, wherein the population of cells is capable of producing rAAV virions comprising the payload nucleic acid sequence at a titer of greater than 1×10¹¹ or no less than 5×10¹¹, 1×10¹², 5×10¹², 1×10¹³ or 1×10¹⁴ viral genomes per milliliter.
[414] The population of cells of any one the preceding embodiments, wherein the population of cells is capable of producing rAAV virions comprising the payload nucleic acid sequence at a concentration of greater than 1×10¹¹ or no less than 5×10¹¹, 1×10¹², 5×10¹², 1×10¹³ or 1×10¹⁴ viral genomes per milliliter prior to purification.
[415] The population of cells of any one the preceding embodiments, wherein the rAAV virions comprising the capsid protein and the payload nucleic acid sequence have an infectivity of no less than 50%, 60%, 70%, 80%, 90%, 95%, or 99% at an MOI of 1×10⁵ vg/target cell or less.
[416] The population of cells of any one the preceding embodiments, wherein the rAAV virions have an increased infectivity compared rAAV virions produced by an otherwise comparable the population of cells capable of producing rAAV virions upon transient transfection at the same MOI.
[417] The population of cells of any one the preceding embodiments, wherein the rAAV virions have at least 1%, 5%, 10%, 15%, 20%, 30%, 40%, or 50% greater infectivity compared rAAV virions produced by an otherwise comparable the population of cells capable of producing rAAV virions upon transient transfection at the same MOI.
[418] The population of cells of any one the preceding embodiments, wherein the rAAV virions have at least 50%, 60%, 70%, 80%, 90%, 95%, 97%, 98%, 99%, or 100% infectivity as compared AAV virions AAV at the same MOI.
[419] The population of cells of any one of the preceding embodiments, wherein the AAV virions are wildtype AAV virions produced by a cell having wildtype AAV.
[420] The population of cells of any one the preceding embodiments, wherein the MOI is 1×10¹, 1×10², 2×10³, 5×10⁴, or 1×10⁵ vg/target cell.
[421] The population of cells of any one the preceding embodiments, wherein the MOI is selected from a range of 1×10¹ to 1×10⁵ vg/target cell.
[422] The population of cells of any one the preceding embodiments, wherein the cells are cryopreserved.
[423] The population of cells of any one the preceding embodiments, wherein the cells are comprised within a vial, flask, syringe, or other suitable cell-storage container.
[424] The population of cells of any one the preceding embodiments, wherein production of rAAV virions is inducible in the absence of a plasmid.
[425] The population of cells of any one the preceding embodiments, wherein expression of AAV Rep and Cap proteins is inducible in the absence of a plasmid.
[426] The population of cells of any one the preceding embodiments, wherein expression of the at least one or more helper proteins is inducible in the absence of a plasmid.
[427] The population of cells of any one the preceding embodiments, wherein production of rAAV virions is inducible in the absence of a transfection agent.
[428] The population of cells of any one the preceding embodiments, wherein expression of AAV Rep and Cap proteins is inducible in the absence of a transfection agent.
[429] The population of cells of any one the preceding embodiments, wherein expression of the at least one or more helper proteins is inducible in the absence of a transfection agent.
[430] A second population of cell produced by expanding the population of cells of any one of the preceding embodiments.
[431] The second population of cells of embodiment 430, wherein expanding the population of cells comprises passaging the population of cells at least three times.
[432] The second population of cells of embodiment 430, wherein expanding the population of cells comprises passaging the population of cells from 3 to 60 times.
[433] The second population of cells of embodiment 430, wherein expanding the population of cells comprises passaging the population of cells at least 4, 5, 6, 7, 8, 9, 10, 15, 20, 25, 30, 35, 40, 45, 50, 55, or 60 times.
[434] A stable cell line comprising the population of cells of any one of the preceding embodiments.
[435] The stable cell line of embodiment 434, wherein the population of cells are derived from a single cell.
[436] A stable cell line comprising the population of cells of any one of the preceding embodiments.
[437] The stable cell line of any one of the preceding embodiments, wherein the population of cells are derived from a single cell.
[438] The stable cell line of any one of the preceding embodiments, wherein the single cell is the of any one of the preceding embodiments.
[439] The stable cell line of any one of the preceding embodiments, wherein at least 70%, 80%, 90%, 95%, 99%, or 100% of the cells of the stable cell line are the population of cells of any one of the preceding embodiments.
[440] A stable cell line derived from the cell of any one of the preceding embodiments.
[441] A stable cell line expanded from the cell of any one of the preceding embodiments.
[442] The stable cell line of any one of the preceding embodiments, wherein the stable cell line is a mammalian stable cell line.
[443] The stable cell line of any one of the preceding embodiments, wherein expression of one or more helper proteins is inducible in the absence of a plasmid.
[444] The stable cell line of any one of the preceding embodiments, wherein expression of one or more helper proteins is inducible in the absence of a transfection agent.
[445] The stable cell line of any one of the preceding embodiments, wherein expression of AAV Rep and Cap proteins is inducible in the absence of a plasmid.
[446] The stable cell line of any one of the preceding embodiments, wherein expression of AAV Rep and Cap proteins is inducible in the absence of a transfection agent.
[447] The stable cell line of any one of the preceding embodiments, wherein production of rAAV virions is inducible in the absence of a plasmid.
[448] The stable cell line of any one of the preceding embodiments, wherein production of rAAV virions is inducible in the absence of a transfection agent.
[449] The stable cell line of any one of the preceding embodiments, wherein the stable cell line is capable of producing rAAV virions comprising the payload nucleic acid sequence at a concentration of greater than 1×10¹¹ or no less than 5×10¹¹, 1×10¹², 5×10¹², 1×10¹³ or 1×10¹⁴ viral genomes per milliliter.
[450] The stable cell line of any one of the preceding embodiments, wherein the stable cell line is capable of producing rAAV virions comprising the payload nucleic acid sequence at a concentration of greater than 1×10¹¹ or no less than 5×10¹¹, 1×10¹², 5×10¹², 1×10¹³ or 1×10¹⁴ viral genomes per milliliter prior to purification.
[451] The stable cell line of any one of the preceding embodiments, wherein the stable cell line is conditionally capable of producing rAAV virions having an encapsidation ratio of no less than 0.5, 0.6, 0.7, 0.8, 0.9, 0.95, 0.97, or 0.99.
[452] The stable cell line of any one of the preceding embodiments, wherein the rAAV virions have an encapsidation ratio of no less than 0.5, 0.6, 0.7, 0.8, 0.9, 0.95, 0.97, or 0.99 prior to purification.
[453] The stable cell line of any one of the preceding embodiments, wherein the rAAV virions comprising the capsid protein and the payload nucleic acid sequence have an infectivity of no less than 50%, 60%, 70%, 80%, 90%, 95%, or 99%, at an MOI of 1×10⁵ vg/target cell or less
[454] The stable cell line of any one of the preceding embodiments, wherein the rAAV virions have an increased infectivity compared rAAV virions produced by an otherwise comparable the population of cells capable of producing rAAV virions upon transient transfection at the same MOI.
[455] The stable cell line of any one of the preceding embodiments, wherein the rAAV virions have at least 1%, 5%, 10%, 15%, 20%, 30%, 40%, or 50% greater infectivity compared rAAV virions produced by an otherwise comparable the population of cells capable of producing rAAV virions upon transient transfection at the same MOI.
[456] The stable cell line of any one of the preceding embodiments, wherein the rAAV virions have at least 50%, 60%, 70%, 80%, 90%, 95%, 97%, 98%, 99%, or 100% infectivity as compared to AAV virions produced by a cell having wildtype AAV at the same MOI.
[457] The stable cell line of any one of the preceding embodiments, wherein the rAAV virions have at least 50%, 60%, 70%, 80%, 90%, 95%, 97%, 98%, 99%, or 100% infectivity as compared to AAV virions at the same MOI.
[458] The stable cell line of any one of the preceding embodiments, wherein the AAV virions are wildtype AAV virions produced by a cell having wildtype AAV.
[459] The stable cell line of any one of the preceding embodiments, wherein the MOI is 1×10¹, 1×10², 2×10³, 5×10⁴, or 1×10⁵ vg/target cell.
[460] The stable cell line of any one of the preceding embodiments, wherein the MOI is selected from a range of 1×10¹ to 1×10⁵ vg/target cell.
[461] The stable cell line of any one of the preceding embodiments, wherein at least one cell of the stable cell line is cryopreserved.
[462] The stable cell line of any one of the preceding embodiments, wherein at least one cell of the stable cell line is in a vial, flask, syringe, or other suitable cell-storage container.
[463] A cell culture composition comprising:
   a) suspension-adapted cells,
   b) serum-free cell culture media, and
   c) recombinant AAV (rAAV) virions,
   wherein the cell culture composition is free of herpes simplex virus, baculovirus, and adenovirus, and
   wherein the cell culture composition is free of plasmid and transfection agent.
[464] The cell culture composition of any one of the preceding embodiments, wherein the cell culture composition is free of polyethylenimine (PEI).
[465] The cell culture composition of any one of the preceding embodiments, wherein the suspension-adapted cells are suspension-adapted mammalian cells.
[466] The cell culture composition of any one of the preceding embodiments, wherein the suspension-adapted cells are suspension-adapted HEK293 cells or derivatives thereof.
[467] The cell culture composition of any one of the preceding embodiments, wherein the suspension-adapted mammalian cells are cells from the stable cell line of any one of the preceding embodiments, the population of cells of any one of the preceding embodiments, or comprise a cell of any one of the preceding embodiments.
[468] The cell culture composition of any one of the preceding embodiments, wherein the cell culture composition has a prepurification rAAV concentration of no less than 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁴, or 5×10¹⁵ viral genome (vg)/L.
[469] The cell culture composition of any one of the preceding embodiments, wherein the cell culture composition has a prepurification rAAV encapsidation ratio of no less than 0.5, 0.6, 0.7, 0.8, 0.9, 0.95, 0.97, or 0.99.
[470] A bioreactor comprising the stable cell line of any one of the preceding embodiments.
[471] A bioreactor comprising the population of cells of any one of the preceding embodiments.
[472] A bioreactor comprising the cell of any one of the preceding embodiments.
[473] A bioreactor containing the cell culture of any one of the preceding embodiments.
[474] The bioreactor of any one of the preceding embodiments, wherein the bioreactor is a 1 L bioreactor.
[475] The bioreactor of any one of the preceding embodiments, wherein the bioreactor is a 1 L bioreactor.
[476] The bioreactor of any one of the preceding embodiments, wherein the bioreactor has a total rAAV yield of greater than 1×10¹⁴ viral genome (vg).
[477] The bioreactor of any one of the preceding embodiments, wherein the bioreactor is a 5 L bioreactor.
[478] The bioreactor of any one of the preceding embodiments, wherein the bioreactor has a total rAAV yield of greater than 5×10¹⁴ viral genome (vg).
[479] The bioreactor of any one of the preceding embodiments, wherein the bioreactor is a 50 L bioreactor.
[480] The bioreactor of any one of the preceding embodiments, wherein the bioreactor has a total rAAV yield of greater than 5×10¹⁵ viral genome (vg).
[481] The bioreactor of any one of the preceding embodiments, wherein the bioreactor is a 100 L bioreactor.
[482] The bioreactor of any one of the preceding embodiments, wherein the bioreactor has a total rAAV yield of greater than 1×10¹⁶ viral genome (vg).
[483] The bioreactor of any one of the preceding embodiments, wherein the bioreactor is a 500 L bioreactor.
[484] The bioreactor of any one of the preceding embodiments, wherein the bioreactor has a total rAAV yield of greater than 5×10¹⁶ viral genome (vg).
[485] The bioreactor of any one of the preceding embodiments, wherein the bioreactor is a 2000 L bioreactor.
[486] The bioreactor of any one of the preceding embodiments, wherein the bioreactor has a total rAAV yield of greater than 2×10¹⁷ viral genome (vg).
[487] A bioreactor comprising a plurality of rAAV virions having a concentration of greater than 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, or 5×10¹⁵ viral genome (vg)/L.
[488] A bioreactor comprising a plurality of rAAV virions having a prepurification concentration of greater than 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, or 5×10¹⁵ viral genome (vg)/L.
[489] The bioreactor of any one of the preceding embodiments, wherein the bioreactor is a 1 L, 5 L, 50 L, 100 L, 500 L, or 2000 L bioreactor.
[490] The bioreactor of any one of the preceding embodiments, wherein the bioreactor is a single use bioreactor.
[491] A composition comprising a plurality of rAAV virions encapsidating a viral genome, wherein the composition has a prepurification concentration of greater than 1×10¹¹ or no less than 5×10¹¹, 1×10¹², 5×10¹², 1×10¹³ or 1×10¹⁴ viral genomes per milliliter.
[492] A composition comprising a plurality of rAAV virions encapsidating a viral genome, wherein the composition has a prepurification encapsidation ratio of no less than 0.5, 0.6, 0.7, 0.8, 0.9, 0.95, 0.97, or 0.99.
[493] A composition comprising an rAAV virion encapsidating a viral genome, wherein the composition has an infectivity of no less than 50%, 60%, 70%, 80%, 90%, 95%, or 99% at an MOI of 1×10⁵ vg/target cell or less.
[494] The composition of any one of the preceding embodiments, wherein the rAAV virion has an increased infectivity compared an rAAV virion produced by an otherwise comparable cell capable of producing rAAV virions upon transient transfection at the same MOI.
[495] The composition of any one of the preceding embodiments, wherein the rAAV virion has at least 1%, 5%, 10%, 15%, 20%, 30%, 40%, or 50% greater infectivity compared an rAAV virion produced by an otherwise comparable cell capable of producing rAAV virions upon transient transfection at the same MOI.
[496] The composition of any one of the preceding embodiments, wherein the rAAV virion has at least 50%, 60%, 70%, 80%, 90%, 95%, 97%, 98%, 99%, or 100% infectivity as compared an AAV virion produced by a cell having wildtype AAV at the same MOI.
[497] The composition of any one of the preceding embodiments, wherein the rAAV virion has at least 50%, 60%, 70%, 80%, 90%, 95%, 97%, 98%, 99%, or 100% infectivity as compared an AAV virion produced by a cell having wildtype AAV at the same MOI.
[498] The composition of embodiment, further comprising a plurality of the rAAV virion.
[499] The composition of any one of the preceding embodiments, wherein the plurality of rAAV virions having a prepurification concentration of greater than 1×10¹¹ or no less than 5×10¹¹, 1×10¹², 5×10¹², 1×10¹³ or 1×10¹⁴ viral genomes per milliliter.
[500] The composition of any one of the preceding embodiments, wherein the plurality of rAAV virions having a prepurification encapsidation ratio of no less than 0.5, 0.6, 0.7, 0.8, 0.9, 0.95, 0.97, or 0.99.
[501] The composition of any one of the preceding embodiments, wherein the plurality of rAAV virions have an infectivity of no less than 50%, 60%, 70%, 80%, 90%, 95%, or 99%.
[502] The composition of any one of the preceding embodiments, wherein the plurality of rAAV virions have an increased infectivity compared a plurality of rAAV virions produced by an otherwise comparable the population of cells capable of producing rAAV virions upon transient transfection at the same MOI.
[503] The composition of any one of the preceding embodiments, wherein the plurality of rAAV virions have at least 1%, 5%, 10%, 15%, 20%, 30%, 40%, or 50% greater infectivity compared a plurality of rAAV virions produced by an otherwise comparable the population of cells capable of producing rAAV virions upon transient transfection at the same MOI.
[504] The composition of any one of the preceding embodiments, wherein the plurality of rAAV virions have at least 50%, 60%, 70%, 80%, 90%, 95%, 97%, 98%, 99%, or 100% infectivity as compared a plurality of AAV virions produced by a cell having wildtype AAV at the same MOI.
[505] The composition of any one of the preceding embodiments, wherein the MOI is 1×10¹, 1×10², 2×10³, 5×10⁴, or 1×10⁵ vg/target cell.
[506] The composition of any one of the preceding embodiments, wherein the MOI is selected from a range of 1×10¹ to 1×10⁵ vg/target cell.
[507] The composition of any one of the preceding embodiments, wherein the viral genome comprises a sequence coding for a payload.
[508] The cell of any one of the preceding embodiments, wherein expression of the sequence of the payload is driven by a constitutive promoter.
[509] The cell of any one of the preceding embodiments, wherein the sequence of the payload comprises a polynucleotide sequence coding for a gene.
[510] The cell of any one of the preceding embodiments, wherein the gene codes for a selectable marker or detectable marker.
[511] The cell of any one of the preceding embodiments, wherein the gene codes for a therapeutic polypeptide or transgene.
[512] The cell of any one of the preceding embodiments, wherein the sequence of the payload comprises a polynucleotide sequence coding for a therapeutic polynucleotide.
[513] The cell of any one of the preceding embodiments, wherein the therapeutic polynucleotide is a tRNA suppressor or a guide RNA.
[514] The cell of any one of the preceding embodiments, wherein the guide RNA is a polyribonucleotide capable of binding to a protein.
[515] The cell of any one of the preceding embodiments, wherein the protein is nuclease.
[516] The cell of any one of the preceding embodiments, wherein the protein is a Cas protein, an ADAR protein, or an ADAT protein.
[517] The cell of any one of the preceding embodiments, wherein the Cas protein is catalytically inactive Cas protein.
[518] The composition of any one of the preceding embodiments, wherein the rAAV virion comprises a Cap polypeptide.
[519] The composition of any one of the preceding embodiments, wherein the Cap polypeptide is an AAV capsid protein.
[520] The composition of any one of the preceding embodiments, wherein the AAV capsid protein is VP1, VP2, or VP3.
[521] The composition of any one of the preceding embodiments, wherein a serotype of the AAV capsid protein is selected from the group consisting of AAV1, AAV2, AAV3, AAV4, AAV5, AAV6, AAV7, AAV8, AAV9, AAV 10, AAV11, AAV 12, AAV13, AAV 14, AAV 15 and AAV 16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, and AAVhu68.
[522] A first composition and a second composition, wherein the first composition comprises the composition of any one of the preceding embodiments and the second composition comprises the composition of any one of the preceding embodiments.
[523] The first composition and the second composition of any one of the preceding embodiments, wherein the first composition and the second composition have an encapsidation ratio that varies by no more than 20%, 10%, 5%, 4%, 3%, 2%, or 1%.
[524] The first composition and the second composition of any one of the preceding embodiments, wherein the first composition and the second composition have a concentration of viral genomes per milliliter that varies by no more than 20%, 10%, 5%, 4%, 3%, 2%, or 1%.
[525] The first composition and the second composition of any one of the preceding embodiments, wherein the first composition and the second composition have an infectivity that varies by no more than 20%, 10%, 5%, 4%, 3%, 2%, or 1%.
[526] The first composition and the second composition of any one of the preceding embodiments, wherein the first composition is a first dose and the second composition is a second dose.
[527] The first composition and the second composition of any one of the preceding embodiments, wherein the first composition is produced at least 1, 2, 3, 4, 5, 6, or 7 days before the second composition is produced.
[528] The first composition and the second composition of any one of the preceding embodiments, wherein a plurality of rAAV virions of the first composition is produced at least 1, 2, 3, 4, 5, 6, or 7 days before a plurality of rAAV virions of the second composition is produced.
[529] The first composition and the second composition of any one of the preceding embodiments, wherein the first composition is produced at least 1, 2, 3, 4, 5, 6, 7, 8, 9, 10, 11, or 12 months before the second composition is produced.
[530] The first composition and the second composition of any one of the preceding embodiments, wherein a plurality of rAAV virions of the first composition is produced at least 1, 2, 3, 4, 5, 6, 7, 8, 9, 10, 11, or 12 months before the second composition is produced.
[531] The first composition and the second composition of any one of the preceding embodiments, wherein the first composition is produced at least 1, 2, 3, 4, 5, 6, 7, 8, 9, 10, 11, or 12 years before the second composition is produced.
[532] The first composition and the second composition of any one of the preceding embodiments, wherein a plurality of rAAV virions of the first composition is produced at least 1, 2, 3, 4, 5, 6, 7, 8, 9, 10, 11, or 12 years before the second composition is produced.
[533] The first composition and the second composition of any one of the preceding embodiments, wherein the first composition is produced from a plurality of virions from a first bioreactor and the second composition is produced from a plurality of virions from a second bioreactor.
[534] The first composition and the second composition of any one of the preceding embodiments, further comprising a third composition.
[535] The first composition and the second composition of any one of the preceding embodiments, wherein the first composition, the second composition, and the third composition have an encapsidation ratio that varies by no more than 20%, 10%, 5%, 4%, 3%, 2%, or 1%.
[536] The first composition and the second composition of any one of the preceding embodiments, wherein the first composition, the second composition, and the third composition have a concentration of viral genomes per milliliter that varies by no more than 20%, 10%, 5%, 4%, 3%, 2%, or 1%.
[537] The first composition and the second composition of any one of the preceding embodiments, wherein the first composition, the second composition, and the third composition have an infectivity that varies by no more than 20%, 10%, 5%, 4%, 3%, 2%, or 1%.
[538] The first composition and the second composition of any one of the preceding embodiments, wherein the third composition is a third dose.
[539] The first composition and the second composition of any one of the preceding embodiments, wherein the third composition is produced at least 1, 2, 3, 4, 5, 6, or 7 days after the second composition is produced.
[540] The first composition and the second composition of any one of the preceding embodiments, wherein a plurality of rAAV virions of the third composition is produced at least 1, 2, 3, 4, 5, 6, or 7 days after a plurality of rAAV virions of the second composition is produced.
[541] The first composition and the second composition of any one of the preceding embodiments, wherein the third composition is produced at least 1, 2, 3, 4, 5, 6, 7, 8, 9, 10, 11, or 12 months after the second composition is produced.
[542] The first composition and the second composition of any one of the preceding embodiments, wherein a plurality of rAAV virions of the third composition is produced at least 1, 2, 3, 4, 5, 6, 7, 8, 9, 10, 11, or 12 months after the second composition is produced.
[543] The first composition and the second composition of any one of the preceding embodiments, wherein the third composition is produced at least 1, 2, 3, 4, 5, 6, 7, 8, 9, 10, 11, or 12 years after the second composition is produced.
[544] The first composition and the second composition of any one of the preceding embodiments, wherein a plurality of rAAV virions of the third composition is produced at least 1, 2, 3, 4, 5, 6, 7, 8, 9, 10, 11, or 12 years after the second composition is produced.
[545] The first composition and the second composition of any one of the preceding embodiments, wherein the third composition is produced from a plurality of virions from a third bioreactor.
[546] A pharmaceutical composition comprising the plurality of rAAV virions of any one of the preceding embodiments and a pharmaceutically acceptable carrier.
[547] A plurality of pharmaceutical doses, wherein each dose independently comprises a pharmaceutical composition of embodiment 546.
[548] The plurality of pharmaceutical doses of embodiment 547, wherein the encapsidation ratio has a difference of not more than 20%, 10%, 5%, 4%, 3%, 2%, or 1% between a first dose and a second dose.
[549] The plurality of pharmaceutical doses of any one of the preceding embodiments, wherein the concentration of viral genomes has a difference of not more than 20%, 10%, 5%, 4%, 3%, 2%, or 1% between a first dose and a second dose.
[550] The plurality of pharmaceutical doses of any one of the preceding embodiments, wherein the concentration of vector genomes has a difference of not more than 20%, 10%, 5%, 4%, 3%, 2%, or 1% between a first dose and a second dose.
[551] The plurality of pharmaceutical doses of any one of the preceding embodiments, wherein the rAAV virion infectivity has a difference of not more than 20%, 10%, 5%, 4%, 3%, 2%, or 1% between a first dose and a second dose.
[552] A method of generating a stable cell line, the method comprising:
   contacting a cell to the polynucleotide construct of any one of embodiments 1-112, wherein the polynucleotide construct of any one embodiments 1-112 stably integrates into the genome of the cell; and
   passaging the cell to generate the stable cell line.
[553] A method of generating a stable cell line, the method comprising:
   contacting a cell to the polynucleotide construct of any one of embodiments 1-112, wherein the polynucleotide construct of any one embodiments 1-112 stably integrates into the genome of the cell;
   contacting the cell to the polynucleotide construct of any one of embodiments 119-209, wherein the polynucleotide construct of any one of embodiments 119-209 stably integrates into the genome of the cell; and
   passaging the cell to generate the stable cell line.
[554] A method of generating a stable cell line, the method comprising:
   contacting a cell to the polynucleotide construct of any one of embodiments 1-112, wherein the polynucleotide construct of any one embodiments 1-112 stably integrates into the genome of the cell;
   contacting the cell to the polynucleotide construct of any one of embodiments 119-209, wherein the polynucleotide construct of any one of embodiments 119-209 stably integrates into the genome of the cell; and
   passaging the cell to generate the stable cell line.
[555] A method of generating a stable cell line, the method comprising:
   contacting a cell to the polynucleotide construct of any one of embodiments 119-209, wherein the polynucleotide construct of any one embodiments 119-209 stably integrates into the genome of the cell;
   contacting the cell to the polynucleotide construct of any one of embodiments 216-271, wherein the polynucleotide construct of any one of embodiments Z stably integrates into the genome of the cell; and
   passaging the cell to generate the stable cell line.
[556] A method of generating a stable cell line, the method comprising:
   contacting a cell to a plurality of the polynucleotide construct of any one of embodiments 1-112, wherein the plurality of polynucleotide construct of any one embodiments 1-112 stably integrates into the genome of the cell;
   contacting the cell to a plurality of the polynucleotide construct of any one of embodiments 119-209, wherein the plurality of the polynucleotide construct of any one of embodiments 119-209 stably integrates into the genome of the cell; and
   passaging the cell to generate the stable cell line.
[557] A method of generating a stable cell line, the method comprising:
   contacting a cell to a plurality of the polynucleotide construct of any one of embodiments 1-112, wherein the plurality of polynucleotide construct of any one embodiments 1-112 stably integrates into the genome of the cell;
   contacting the cell to a plurality of the polynucleotide construct of any one of embodiments 216-271, wherein the plurality of the polynucleotide construct of any one of embodiments 216-271 stably integrates into the genome of the cell; and
   passaging the cell to generate the stable cell line.
[558] A method of generating a stable cell line, the method comprising:
   contacting a cell to a plurality of the polynucleotide construct of any one of embodiments 216-271 wherein the plurality of polynucleotide construct of any one embodiments 216-271 stably integrates into the genome of the cell;
   contacting the cell to a plurality of the polynucleotide construct of any one of embodiments 119-209, wherein the plurality of the polynucleotide construct of any one of embodiments 119-209 stably integrates into the genome of the cell; and
   passaging the cell to generate the stable cell line.
[559] A method of generating a stable cell line, the method comprising:
   contacting a cell to a plurality of the polynucleotide construct of any one of embodiments 1-112, wherein the plurality of polynucleotide construct of any one embodiments 1-112 stably integrates into the genome of the cell;
   contacting the cell to a plurality of the polynucleotide construct of any one of embodiments 119-209, wherein the plurality of the polynucleotide construct of any one of embodiments 119-209 stably integrates into the genome of the cell;
   contacting the cell to a plurality of the polynucleotide construct of any one of embodiments 216-271, wherein the plurality of the polynucleotide construct of any one of embodiments 216-271 stably integrates into the genome of the cell; and
   passaging the cell to generate the stable cell line.
[560] A method of generating a stable cell line, the method comprising:
   contacting a cell to a plurality of the polynucleotide construct of any one of embodiments 216-271 wherein the plurality of polynucleotide construct of any one embodiments 216-271 stably integrates into the genome of the cell;
   contacting the cell to a plurality of the polynucleotide construct of any one of embodiments 1-112, wherein the plurality of the polynucleotide construct of any one of embodiments 1-112 stably integrates into the genome of the cell;
   contacting the cell to a plurality of the polynucleotide construct of any one of embodiments 119-209, wherein the plurality of the polynucleotide construct of any one of embodiments 119-209 stably integrates into the genome of the cell; and
   passaging the cell to generate the stable cell line.
[561] The method of any one of the preceding embodiments, wherein the polynucleotide construct of any one embodiments 1-112 and the polynucleotide construct of any one embodiments 119-209 separately integrate into the genome of the cell.
[562] The method of any one of the preceding embodiments, wherein the plurality of the polynucleotide construct of any one embodiments 1-112 and the plurality of the polynucleotide construct of any one embodiments 119-209 separately integrate into the genome of the cell.
[563] The method of any one of the preceding embodiments, wherein the polynucleotide construct of any one embodiments 216-271 and the polynucleotide construct of any one embodiments 119-209 separately integrate into the genome of the cell.
[564] The method of any one of the preceding embodiments, wherein the plurality of the polynucleotide construct of any one embodiments 216-271 and the plurality of the polynucleotide construct of any one embodiments 119-209 separately integrate into the genome of the cell.
[565] The method of any one of the preceding embodiments, wherein the polynucleotide construct of any one embodiments 1-112 and the polynucleotide construct of any one embodiments 216-271 separately integrate into the genome of the cell.
[566] The method of any one of the preceding embodiments, wherein the plurality of the polynucleotide construct of any one embodiments 1-112 and the plurality of the polynucleotide construct of any one embodiments 216-271 separately integrate into the genome of the cell.
[567] The method of any one of the preceding embodiments, wherein the polynucleotide construct of any one embodiments 1-112, the polynucleotide construct of any of embodiments 216-271, and the polynucleotide construct of any one embodiments 119-209 separately integrate into the genome of the cell.
[568] The method of any one of the preceding embodiments, wherein the plurality of the polynucleotide construct of any one embodiments 1-112, the plurality of any one of embodiments 216-271, and the plurality of the polynucleotide construct of any one embodiments 119-209 separately integrate into the genome of the cell.
[569] The method of any one of the preceding embodiments, further comprising contacting the cell to a payload construct.
[570] The method of any one of the preceding embodiments, wherein the payload construct stably integrates into the genome of the cell.
[571] The method of any one of the preceding embodiments, wherein the polynucleotide construct of any one embodiments 1-112 and the payload construct separately integrate into the cell.
[572] The method of any one of the preceding embodiments, wherein the polynucleotide construct of any one embodiments 119-209 and the payload construct separately integrate into the cell.
[573] The method of any one of the preceding embodiments, wherein the polynucleotide construct of any one embodiments 216-271 and the payload construct separately integrate into the cell.
[574] The method of any one of the preceding embodiments, wherein the polynucleotide construct of any one embodiments 1-112, the polynucleotide construct of any one embodiments 119-209, and the payload construct separately integrate into the cell.
[575] The method of any one of the preceding embodiments, wherein the polynucleotide construct of any one embodiments 1-112, the polynucleotide construct of any one embodiments 216-271, and the payload construct separately integrate into the cell.
[576] The method of any one of the preceding embodiments, wherein the polynucleotide construct of any one embodiments 216-271, the polynucleotide construct of any one embodiments 119-209, and the payload construct separately integrate into the cell.
[577] The method of any one of the preceding embodiments, wherein the polynucleotide construct of any one of embodiments 1-112, the polynucleotide construct of any one embodiments 216-271, the polynucleotide construct of any one embodiments 119-209, and the payload construct separately integrate into the cell.
[578] The method of any one of the preceding embodiments, further comprising contacting the cell to a plurality of a payload construct.
[579] The method of any one of the preceding embodiments, wherein the plurality of the payload construct stably integrates into the genome of the cell.
[580] The method of any one of the preceding embodiments, wherein the plurality of the polynucleotide construct of any one embodiments 1-112 and the plurality of the payload construct separately integrate into the cell.
[581] The method of any one of the preceding embodiments, wherein the plurality of the polynucleotide construct of any one embodiments 119-209 and the plurality of the payload construct separately integrate into the cell.
[582] The method of any one of the preceding embodiments, wherein the plurality of the polynucleotide construct of any one embodiments 216-271 and the plurality of the payload construct separately integrate into the cell.
[583] The method of any one of the preceding embodiments, wherein the plurality of the polynucleotide construct of any one embodiments 1-112, the plurality of the polynucleotide construct of any one embodiments 119-209, and the plurality of the payload construct separately integrate into the cell.
[584] The method of any one of the preceding embodiments, wherein the plurality of the polynucleotide construct of any one embodiments 1-112, the plurality of the polynucleotide construct of any one embodiments 216-271, and the plurality of the payload construct separately integrate into the cell.
[585] The method of any one of the preceding embodiments, wherein the plurality of the polynucleotide construct of any one embodiments 216-271, the plurality of the polynucleotide construct of any one embodiments 119-209, and the plurality of the payload construct separately integrate into the cell.
[586] The method of any one of the preceding embodiments, wherein the plurality of the polynucleotide construct of any one of embodiments 1-112, the plurality of the polynucleotide construct of any one embodiments 216-271, the plurality of the polynucleotide construct of any one embodiments 119-209, and the plurality of the payload construct of any one of the preceding embodiments separately integrate into the cell.
[587] The method of any one of the preceding embodiments, wherein the passaging is in a cell media comprising a selective pressure.
[588] The method of any one of the preceding embodiments, wherein the passaging is in a cell media comprising at least two selective pressures.
[589] The method of any one of the preceding embodiments, wherein the selective pressure is an antibiotic.
[590] The method of any one of the preceding embodiments, wherein the antibiotic is blasticidin or puromycin.
[591] The method of any one of the preceding embodiments, wherein the passaging is in a cell media comprising at least two antibiotics.
[592] The method of any one of the preceding embodiments, wherein the selection pressure is a lack of a nutrient.
[593] The method of any one the preceding embodiments, wherein the lack of a nutrient is a lack of hypoxanthine and a lack of thymidine.
[594] The method of any one of the preceding embodiments, wherein the stable cell line is capable of reaching a viable cell density of no less than 1×10⁶, 2×10⁶, 5×10⁶, or 1×10⁷ cells per milliliter.
[595] A method of generating a stable cell, the method comprising:
   contacting a cell to the polynucleotide construct of any one of embodiments 1-112, wherein the polynucleotide construct of any one embodiments 1-112 stably integrates into the genome of the cell
[596] A method of generating a stable cell, the method comprising:
   contacting a cell to the polynucleotide construct of any one of embodiments 1-112, wherein the polynucleotide construct of any one embodiments 1-112 stably integrates into the genome of the cell; and
   contacting the cell to the polynucleotide construct of any one of embodiments 119-209, wherein the polynucleotide construct of any one of embodiments 119-209 stably integrates into the genome of the cell.
[597] A method of generating a stable cell, the method comprising:
   contacting a cell to the polynucleotide construct of any one of embodiments 1-112, wherein the polynucleotide construct of any one embodiments 1-112 stably integrates into the genome of the cell; and
   contacting the cell to the polynucleotide construct of any one of embodiments 216-271, wherein the polynucleotide construct of any one of embodiments 216-271 stably integrates into the genome of the cell.
[598] A method of generating a stable cell, the method comprising:
   contacting a cell to the polynucleotide construct of any one of embodiments 119-209, wherein the polynucleotide construct of any one embodiments 119-209 stably integrates into the genome of the cell; and
   contacting the cell to the polynucleotide construct of any one of embodiments 216-271, wherein the polynucleotide construct of any one of embodiments 216-271 stably integrates into the genome of the cell.
[599] A method of generating a stable cell, the method comprising:
   contacting a cell to a plurality of the polynucleotide construct of any one of embodiments 1-112, wherein the plurality of polynucleotide construct of any one embodiments 1-112 stably integrates into the genome of the cell; and
   contacting the cell to a plurality of the polynucleotide construct of any one of embodiments 119-209, wherein the plurality of the polynucleotide construct of any one of embodiments 119-209 stably integrates into the genome of the cell.
[600] A method of generating a stable cell, the method comprising:
   contacting a cell to a plurality of the polynucleotide construct of any one of embodiments 1-112, wherein the plurality of polynucleotide construct of any one embodiments 1-112 stably integrates into the genome of the cell; and
   contacting the cell to a plurality of the polynucleotide construct of any one of embodiments 216-271, wherein the plurality of the polynucleotide construct of any one of embodiments 216-271 stably integrates into the genome of the cell.
[601] A method of generating a stable cell, the method comprising:
   contacting a cell to a plurality of the polynucleotide construct of any one of embodiments 216-271 wherein the plurality of polynucleotide construct of any one embodiments 216-271 stably integrates into the genome of the cell; and
   contacting the cell to a plurality of the polynucleotide construct of any one of embodiments 119-209, wherein the plurality of the polynucleotide construct of any one of embodiments 119-209 stably integrates into the genome of the cell.
[602] A method of generating a stable cell, the method comprising:
   contacting a cell to a plurality of the polynucleotide construct of any one of embodiments 1-112, wherein the plurality of polynucleotide construct of any one embodiments 1-112 stably integrates into the genome of the cell;
   contacting the cell to a plurality of the polynucleotide construct of any one of embodiments 119-209, wherein the plurality of the polynucleotide construct of any one of embodiments 119-209 stably integrates into the genome of the cell; and
   contacting the cell to a plurality of the polynucleotide construct of any one of embodiments 216-271, wherein the plurality of the polynucleotide construct of any one of embodiments 216-271 stably integrates into the genome of the cell.
[603] A method of generating a stable cell, the method comprising:
   contacting a cell to a plurality of the polynucleotide construct of any one of embodiments 216-271 wherein the plurality of polynucleotide construct of any one embodiments 216-271 stably integrates into the genome of the cell;
   contacting the cell to a plurality of the polynucleotide construct of any one of embodiments 1-112, wherein the plurality of the polynucleotide construct of any one of embodiments 1-112 stably integrates into the genome of the cell; and
   contacting the cell to a plurality of the polynucleotide construct of any one of embodiments 119-209, wherein the plurality of the polynucleotide construct of any one of embodiments 119-209 stably integrates into the genome of the cell.
[604] A method of generating a population of stable cells, the method comprising:
   contacting a cell to the polynucleotide construct of any one of embodiments 1-112, wherein the polynucleotide construct of any one embodiments 1-112 stably integrates into the genome of the cell; and
   passaging the cell to generate the population of stable cells.
[605] A method of generating a population of stable cells, the method comprising:
   contacting a cell to the polynucleotide construct of any one of embodiments 1-112, wherein the polynucleotide construct of any one embodiments 1-112 stably integrates into the genome of the cell;
   contacting the cell to the polynucleotide construct of any one of embodiments 119-209, wherein the polynucleotide construct of any one of embodiments 119-209 stably integrates into the genome of the cell; and
   passaging the cell to generate the population of stable cells.
[606] A method of generating a population of stable cells, the method comprising:
   contacting a cell to the polynucleotide construct of any one of embodiments 1-112, wherein the polynucleotide construct of any one embodiments 1-112 stably integrates into the genome of the cell;
   contacting the cell to the polynucleotide construct of any one of embodiments 216-271, wherein the polynucleotide construct of any one of embodiments 216-271 stably integrates into the genome of the cell; and passaging the cell to generate the population of stable cells.
[607] A method of generating a population of stable cells, the method comprising:
   contacting a cell to the polynucleotide construct of any one of embodiments 119-209, wherein the polynucleotide construct of any one embodiments 119-209 stably integrates into the genome of the cell;
   contacting the cell to the polynucleotide construct of any one of embodiments 216-271, wherein the polynucleotide construct of any one of embodiments 216-271 stably integrates into the genome of the cell; and
   passaging the cell to generate the population of stable cells.
[608] A method of generating a population of stable cells, the method comprising:
   contacting a cell to a plurality of the polynucleotide construct of any one of embodiments 1-112, wherein the plurality of polynucleotide construct of any one embodiments 1-112 stably integrates into the genome of the cell;
   contacting the cell to a plurality of the polynucleotide construct of any one of embodiments 119-209, wherein the plurality of the polynucleotide construct of any one of embodiments 119-209 stably integrates into the genome of the cell; and
   passaging the cell to generate the population of stable cells.
[609] A method of generating a population of stable cells, the method comprising:
   contacting a cell to a plurality of the polynucleotide construct of any one of embodiments 1-112, wherein the plurality of polynucleotide construct of any one embodiments 1-112 stably integrates into the genome of the cell;
   contacting the cell to a plurality of the polynucleotide construct of any one of embodiments 216-271, wherein the plurality of the polynucleotide construct of any one of embodiments 216-271 stably integrates into the genome of the cell; and
   passaging the cell to generate the population of stable cells.
[610] A method of generating a population of stable cells, the method comprising:
   contacting a cell to a plurality of the polynucleotide construct of any one of embodiments 216-271 wherein the plurality of polynucleotide construct of any one embodiments 216-271 stably integrates into the genome of the cell;
   contacting the cell to a plurality of the polynucleotide construct of any one of embodiments 119-209, wherein the plurality of the polynucleotide construct of any one of embodiments 119-209 stably integrates into the genome of the cell; and
   passaging the cell to generate the population of stable cells.
[611] A method of generating a population of stable cells, the method comprising:
   contacting a cell to a plurality of the polynucleotide construct of any one of embodiments 1-112, wherein the plurality of polynucleotide construct of any one embodiments 1-112 stably integrates into the genome of the cell;
   contacting the cell to a plurality of the polynucleotide construct of any one of embodiments 119-209, wherein the plurality of the polynucleotide construct of any one of embodiments 119-209 stably integrates into the genome of the cell;
   contacting the cell to a plurality of the polynucleotide construct of any one of embodiments 216-271, wherein the plurality of the polynucleotide construct of any one of embodiments 216-271 stably integrates into the genome of the cell; and
   passaging the cell to generate the population of stable cells.
[612] A method of generating a population of stable cells, the method comprising:
   contacting a cell to a plurality of the polynucleotide construct of any one of embodiments 216-271 wherein the plurality of polynucleotide construct of any one embodiments 216-271 stably integrates into the genome of the cell;
   contacting the cell to a plurality of the polynucleotide construct of any one of embodiments 1-112, wherein the plurality of the polynucleotide construct of any one of embodiments 1-112 stably integrates into the genome of the cell;
   contacting the cell to a plurality of the polynucleotide construct of any one of embodiments 119-209, wherein the plurality of the polynucleotide construct of any one of embodiments 119-209 stably integrates into the genome of the cell; and
   passaging the cell to generate the population of stable cells.
[613] The method of any one of the preceding embodiments, wherein the population of stable cells is capable of reaching a viable cell density of no less than 1×10⁶, 2×10⁶, 5×10⁶, or 1×10⁷ cells per milliliter.
[614] A method of generating a stable cell line, the method comprising:
   contacting a cell to the polynucleotide construct of any one of embodiments 1-112, wherein the polynucleotide construct of any one embodiments 1-112 stably integrates into the genome of the cell; and
   passaging the cell to generate the stable cell line.
[615] A method of generating a stable cell line, the method comprising:
   contacting a cell to the polynucleotide construct of any one of embodiments 1-112, wherein the polynucleotide construct of any one embodiments 1-112 stably integrates into the genome of the cell;
   contacting the cell to the polynucleotide construct of any one of embodiments 119-209, wherein the polynucleotide construct of any one of embodiments 119-209 stably integrates into the genome of the cell; and
   passaging the cell to generate the stable cell line.
[616] A method of generating a stable cell line, the method comprising:
   contacting a cell to the polynucleotide construct of any one of embodiments 1-112, wherein the polynucleotide construct of any one embodiments 1-112 stably integrates into the genome of the cell;
   contacting the cell to the polynucleotide construct of any one of embodiments 216-271, wherein the polynucleotide construct of any one of embodiments 216-271 stably integrates into the genome of the cell; and
   passaging the cell to generate the stable cell line.
[617] A method of generating a stable cell line, the method comprising:
   contacting a cell to the polynucleotide construct of any one of embodiments 119-209, wherein the polynucleotide construct of any one embodiments 119-209 stably integrates into the genome of the cell;
   contacting the cell to the polynucleotide construct of any one of embodiments 216-271, wherein the polynucleotide construct of any one of embodiments 216-271 stably integrates into the genome of the cell; and
   passaging the cell to generate the stable cell line.
[618] A method of generating a stable cell line, the method comprising:
   contacting a cell to a plurality of the polynucleotide construct of any one of embodiments 1-112, wherein the plurality of polynucleotide construct of any one embodiments 1-112 stably integrates into the genome of the cell;
   contacting the cell to a plurality of the polynucleotide construct of any one of embodiments 119-209, wherein the plurality of the polynucleotide construct of any one of embodiments 119-209 stably integrates into the genome of the cell; and
   passaging the cell to generate the stable cell line.
[619] A method of generating a stable cell line, the method comprising:
   contacting a cell to a plurality of the polynucleotide construct of any one of embodiments 1-112, wherein the plurality of polynucleotide construct of any one embodiments 1-112 stably integrates into the genome of the cell;
   contacting the cell to a plurality of the polynucleotide construct of any one of embodiments 216-271, wherein the plurality of the polynucleotide construct of any one of embodiments 216-271 stably integrates into the genome of the cell; and
   passaging the cell to generate the stable cell line.
[620] A method of generating a stable cell line, the method comprising:
   contacting a cell to a plurality of the polynucleotide construct of any one of embodiments 216-271 wherein the plurality of polynucleotide construct of any one embodiments 216-271 stably integrates into the genome of the cell;
   contacting the cell to a plurality of the polynucleotide construct of any one of embodiments 119-209, wherein the plurality of the polynucleotide construct of any one of embodiments 119-209 stably integrates into the genome of the cell; and
   passaging the cell to generate the stable cell line.
[621] A method of generating a stable cell line, the method comprising:
   contacting a cell to a plurality of the polynucleotide construct of any one of embodiments 1-112, wherein the plurality of polynucleotide construct of any one embodiments 1-112 stably integrates into the genome of the cell;
   contacting the cell to a plurality of the polynucleotide construct of any one of embodiments 119-209, wherein the plurality of the polynucleotide construct of any one of embodiments 119-209 stably integrates into the genome of the cell;
   contacting the cell to a plurality of the polynucleotide construct of any one of embodiments 216-271, wherein the plurality of the polynucleotide construct of any one of embodiments 216-271 stably integrates into the genome of the cell; and
   passaging the cell to generate the stable cell line.
[622] A method of generating a stable cell line, the method comprising:
   contacting a cell to a plurality of the polynucleotide construct of any one of embodiments 216-271 wherein the plurality of polynucleotide construct of any one embodiments 216-271 stably integrates into the genome of the cell;
   contacting the cell to a plurality of the polynucleotide construct of any one of embodiments 1-112, wherein the plurality of the polynucleotide construct of any one of embodiments 1-112 stably integrates into the genome of the cell;
   contacting the cell to a plurality of the polynucleotide construct of any one of embodiments 119-209, wherein the plurality of the polynucleotide construct of any one of embodiments 119-209 stably integrates into the genome of the cell; and
   passaging the cell to generate the stable cell line.
[623] The method of any one of the preceding embodiments, wherein the polynucleotide construct of any one embodiments 1-112 and the polynucleotide construct of any one embodiments 119-209 separately integrate into the genome of the cell.
[624] The method of any one of the preceding embodiments, wherein the plurality of the polynucleotide construct of any one embodiments 1-112 and the plurality of the polynucleotide construct of any one embodiments 119-209 separately integrate into the genome of the cell.
[625] The method of any one of the preceding embodiments, wherein the polynucleotide construct of any one embodiments 216-271 and the polynucleotide construct of any one embodiments 119-209 separately integrate into the genome of the cell.
[626] The method of any one of the preceding embodiments, wherein the plurality of the polynucleotide construct of any one embodiments 216-271 and the plurality of the polynucleotide construct of any one embodiments 119-209 separately integrate into the genome of the cell.
[627] The method of any one of the preceding embodiments, wherein the polynucleotide construct of any one embodiments 1-112 and the polynucleotide construct of any one embodiments 216-271 separately integrate into the genome of the cell.
[628] The method of any one of the preceding embodiments, wherein the plurality of the polynucleotide construct of any one embodiments 1-112 and the plurality of the polynucleotide construct of any one embodiments 216-271 separately integrate into the genome of the cell.
[629] The method of any one of the preceding embodiments, wherein the polynucleotide construct of any one embodiments 1-112, the polynucleotide construct of any of embodiments 216-271, and the polynucleotide construct of any one embodiments 119-209 separately integrate into the genome of the cell.
[630] The method of any one of the preceding embodiments, wherein the plurality of the polynucleotide construct of any one embodiments 1-112, the plurality of any one of embodiments 216-271, and the plurality of the polynucleotide construct of any one embodiments Y separately integrate into the genome of the cell.
[631] The method of any one of the preceding embodiments, further comprising contacting the cell to a payload construct.
[632] The method of any one of the preceding embodiments, wherein the payload construct stably integrates into the genome of the cell.
[633] The method of any one of the preceding embodiments, wherein the polynucleotide construct of any one embodiments 1-112 and the payload construct separately integrate into the cell.
[634] The method of any one of the preceding embodiments, wherein the polynucleotide construct of any one embodiments 119-209 and the payload construct separately integrate into the cell.
[635] The method of any one of the preceding embodiments, wherein the polynucleotide construct of any one embodiments 216-271 and the payload construct separately integrate into the cell.
[636] The method of any one of the preceding embodiments, wherein the polynucleotide construct of any one embodiments 1-112, the polynucleotide construct of any one embodiments 119-209, and the payload construct separately integrate into the cell.
[637] The method of any one of the preceding embodiments, wherein the polynucleotide construct of any one embodiments 1-112, the polynucleotide construct of any one embodiments 216-271, and the payload construct separately integrate into the cell.
[638] The method of any one of the preceding embodiments, wherein the polynucleotide construct of any one embodiments 216-271, the polynucleotide construct of any one embodiments 119-209, and the payload construct separately integrate into the cell.
[639] The method of any one of the preceding embodiments, wherein the polynucleotide construct of any one of embodiments 1-112, the polynucleotide construct of any one embodiments 216-271, the polynucleotide construct of any one embodiments 119-209, and the payload construct separately integrate into the cell.
[640] The method of any one of the preceding embodiments, further comprising contacting the cell to a plurality of a payload construct.
[641] The method of any one of the preceding embodiments, wherein the plurality of the payload construct stably integrates into the genome of the cell.
[642] The method of any one of the preceding embodiments, wherein the plurality of the polynucleotide construct of any one embodiments 1-112 and the plurality of the payload construct separately integrate into the cell.
[643] The method of any one of the preceding embodiments, wherein the plurality of the polynucleotide construct of any one embodiments 119-209 and the plurality of the payload construct separately integrate into the cell.
[644] The method of any one of the preceding embodiments, wherein the plurality of the polynucleotide construct of any one embodiments 216-271 and the plurality of the payload construct separately integrate into the cell.
[645] The method of any one of the preceding embodiments, wherein the plurality of the polynucleotide construct of any one embodiments 1-112, the plurality of the polynucleotide construct of any one embodiments 119-209, and the plurality of the payload construct separately integrate into the cell.
[646] The method of any one of the preceding embodiments, wherein the plurality of the polynucleotide construct of any one embodiments 1-112, the plurality of the polynucleotide construct of any one embodiments 216-271, and the plurality of the payload construct separately integrate into the cell.
[647] The method of any one of the preceding embodiments, wherein the plurality of the polynucleotide construct of any one embodiments 216-271, the plurality of the polynucleotide construct of any one embodiments 119-209, and the plurality of the payload construct separately integrate into the cell.
[648] The method of any one of the preceding embodiments, wherein the plurality of the polynucleotide construct of any one of embodiments 1-112, the plurality of the polynucleotide construct of any one embodiments 216-271, the plurality of the polynucleotide construct of any one embodiments 119-209, and the plurality of the payload construct separately integrate into the cell.
[649] The cell of any one of the preceding embodiments, wherein the payload construct comprises a sequence of a payload flanked by ITR sequences.
[650] The cell of any one of the preceding embodiments, wherein expression of the sequence of the payload is driven by a constitutive promoter.
[651] The cell of any one of the preceding embodiments, wherein the constitutive promoter and sequence of the payload are flanked by ITR sequences.
[652] The cell of any one of the preceding embodiments, wherein the sequence of the payload comprises a polynucleotide sequence coding for a gene.
[653] The cell of any one of the preceding embodiments, wherein the gene codes for a selectable marker or detectable marker.
[654] The cell of any one of the preceding embodiments, wherein the gene codes for a therapeutic polypeptide or transgene.
[655] The cell of any one of the preceding embodiments, wherein the sequence of the payload comprises a polynucleotide sequence coding for a therapeutic polynucleotide.
[656] The cell of any one of the preceding embodiments, wherein the therapeutic polynucleotide is a tRNA suppressor or a guide RNA.
[657] The cell of any one of the preceding embodiments, wherein the guide RNA is a polyribonucleotide capable of binding to a protein.
[658] The cell of any one of the preceding embodiments, wherein the protein is nuclease.
[659] The cell of any one of the preceding embodiments, wherein the protein is a Cas protein, an ADAR protein, or an ADAT protein.
[660] The cell of any one of the preceding embodiments, wherein the Cas protein is catalytically inactive Cas protein.
[661] The method of any one of the preceding embodiments, wherein the passaging is in a cell media comprising a selective pressure.
[662] The method of any one of the preceding embodiments, wherein the passaging is in a cell media comprising at least two selective pressures.
[663] The method of any one of the preceding embodiments, wherein the selective pressure is an antibiotic.
[664] The method of any one of the preceding embodiments, wherein the antibiotic is blasticidin or puromycin.
[665] The method of any one of the preceding embodiments, wherein the passaging is in a cell media comprising at least two antibiotics.
[666] The method of any one of the preceding embodiments, wherein the selection pressure is a lack of a nutrient.
[667] The method of any one the preceding embodiments, wherein the lack of a nutrient is a lack of hypoxanthine and a lack of thymidine.
[668] The method of any one of the preceding embodiments, wherein the stable cell line is capable of reaching a viable cell density of no less than 1×10⁶, 2×10⁶, 5×10⁶, or 1×10⁷ cells per milliliter.
[669] A method of inducing the cell of any one of the preceding embodiments, the population of cells of any one of embodiments, or the stable cell line of any one of embodiments, the method comprising administering a first triggering agent to the cell, population of cells, or the stable cell line, thereby inducing expression of the Rep polypeptides, Cap polypeptides, and one or more adenoviral helper proteins, in the cell, population of cells, or stable cell line.
[670] The method of any one of the preceding embodiments, wherein the first triggering agent binds to an activator or a repressor.
[671] The method of any one of the preceding embodiments, wherein activation of an inducible promoter is induced.
[672] The method of any one of the preceding embodiments, wherein the activated inducible promoter transcribes a recombinase.
[673] The method of any one of the preceding embodiments, wherein the first triggering agent is tetracycline or cumate.
[674] The method of any one of the preceding embodiments, wherein the tetracycline is doxycycline.
[675] The method of any one of the preceding embodiments, further comprising culturing the cell, population of cells, or the stable cell line with a second triggering agent.
[676] The method of any one of the preceding embodiments, wherein the second triggering agent is an estrogen receptor ligand.
[677] The method of any one of the preceding embodiments, wherein the second triggering agent is a selective estrogen receptor modulator (SERM).
[678] The method of any one of the preceding embodiments, wherein the second triggering agent is tamoxifen.
[679] The method of any one of the preceding embodiments, wherein the second triggering agent binds to the recombinase.
[680] The method of any one of the preceding embodiments, wherein the second triggering agent induces the recombinase to translocate to a nucleus of the cell, of a cell of the population of cells, of a cell of the stable cell lines.
[681] A method of producing rAAV virion, the method comprising
   administering a first triggering agent to the cell, population of cells, or the stable cell line,
   administering a second triggering agent to the cell, population of cells, or stable cell line,
   thereby producing the rAAV virion in the cell, population of cells, or stable cell line.
[682] The method of any one of the preceding embodiments, wherein the first triggering agent binds to an activator or a repressor.
[683] The method of any one of the preceding embodiments, wherein activation of an inducible promoter is induced.
[684] The method of any one of the preceding embodiments, wherein the activated inducible promoter transcribes a recombinase.
[685] The method of any one of the preceding embodiments, wherein the first triggering agent is tetracycline or cumate.
[686] The method of any one of the preceding embodiments, wherein the tetracycline is doxycycline.
[687] The method of any one of the preceding embodiments, further comprising culturing the cell, population of cells, or the stable cell line with a second triggering agent.
[688] The method of any one of the preceding embodiments, wherein the second triggering agent is an estrogen receptor ligand.
[689] The method of any one of the preceding embodiments, wherein the second triggering agent is a selective estrogen receptor modulator (SERM).
[690] The method of any one of the preceding embodiments, wherein the second triggering agent is tamoxifen.
[691] The method of any one of the preceding embodiments, wherein the second triggering agent binds to the recombinase.
[692] The method of any one of the preceding embodiments, wherein the second triggering agent induces the recombinase to translocate to a nucleus of the cell, of a cell of the population of cells, of a cell of the stable cell lines.
[693] The method of any one of the preceding embodiments, wherein the recombinase cuts at recombination sites.
[694] The method of any one of the preceding embodiments, wherein the at least one adenoviral help proteins, the Rep polypeptides, and the Cap polypeptides are expressed.
[695] The method of any one of the preceding embodiments, wherein the Rep polypeptides and the Cap polypeptides assemble into an rAAV virion.
[696] The method of any one of the preceding embodiments, wherein the rAAV virion encapsidates a sequence of a payload.
[697] The method of any one of the preceding embodiments, wherein the cell, population of cells, or stable cell line do not express cytotoxic levels of Rep polypeptides prior to administration of both the first triggering agent and the second triggering agent.
[698] The method of any one of the preceding embodiments, wherein the cell, population of cells, or stable cell line do not express cytotoxic levels of Cap polypeptides prior to administration of both the first triggering agent and the second triggering agent.
[699] The method of any one of the preceding embodiments, wherein the cell, population of cells, or stable cell line do not express cytostatic levels of Rep polypeptides prior to administration of both the first triggering agent and the second triggering agent.
[700] The method of any one of the preceding embodiments, wherein the average concentration of Rep polypeptides within the cell, population of cells, or stable cell line is less than the amount prior to administration of both of the first triggering agent and second triggering agent.
[701] The method of any one of the preceding embodiments, wherein expression of Rep polypeptides and Cap polypeptides becomes constitutive after administration of both the first triggering agent and the second triggering agent.
[702] The method of any one of the preceding embodiments, further comprising performing at least a portion of the method in a bioreactor.
[703] The method of any one of the preceding embodiments, wherein the bioreactor is not less than 20 L, 30, L, 40 L, 50 L, 100 L, 250 L, 300 L, or 500 L.
[704] The method of any one of the preceding embodiments, further comprising producing the rAAV virions in a plurality of batches.
[705] The method of any one of the preceding embodiments, further comprising producing the rAAV virions having a difference in the payload encapsidation ratio of not more than 20%, 15%, 10%, 5%, 3%, 2%, or 1% between a first batch and a second batch.
[706] The method of any one of the preceding embodiments, further comprising producing the rAAV virions having a difference in the concentration of viral genomes of not more than 20%, 15%, 10%, 5%, 3%, 2%, or 1% between a first batch and a second batch.
[707] The method of any one of the preceding embodiments, further comprising producing the rAAV virions having a difference in the concentration of vector genomes of not more than 20%, 15%, 10%, 5%, 3%, 2%, or 1% between a first batch and a second batch.
[708] The method of any one of the preceding embodiments, further comprising producing the rAAV virions having a difference in infectivity of not more than 20%, 15%, 10%, 5%, 3%, 2%, or 1% between a first batch and a second batch.
[709] The method of any one of the preceding embodiments, further comprising performing the method according to good manufacturing practice (GMP) standards.
[710] The method of any one of the preceding embodiments, further comprising performing the method in a GMP facility.
[711] The method of any one of the preceding embodiments, further comprising culturing the cells in a culture medium and collecting a portion of the plurality of rAAV virions from the culture medium.
[712] The method of any one of the preceding embodiments, further comprising purifying at least some of the plurality of rAAV virions collected from the culture medium to obtain a purified rAAV population.
[713] The method of any one of the preceding embodiments, wherein the purifying comprises performing chromatographic purification.
[714] The method of any one of the preceding embodiments, wherein the chromatographic purification comprises using a positively charged anion exchange resin, using a negatively charged anion exchange resin, using cation exchange chromatography, using affinity chromatography, using size exclusion chromatography, or a combination thereof.
[715] The method of any one of the preceding embodiments, wherein the chromatographic purification comprises using column chromatographic fractionation.
[716] A method of treating a condition or disorder, the method comprising administering a therapeutically effective amount of the pharmaceutical composition of any one of the preceding embodiments to a patient in need thereof.
[717] The method of embodiment 716, wherein the disorder is a monogenic disorder.
[718] The method of any one of embodiments 716-717, wherein the treatment results in at least one undesirable side effect and wherein the undesirable side effect is reduced relative to administering a daily dose that deviates more than 50%, 40%, 30%, 30%, 15%, 10%, 5%, or 2% from an expected dose.
[719] The method of any one of embodiments 716-719, wherein the administering is by injection.
[720] The method of embodiment 719, wherein the injection is an infusion.
[721] The method of any one of embodiments 716-720, wherein the daily dose is administered to the patient once.
[722] The method of any one of embodiments 716-720, wherein the daily dose is administered to the patient two or more times.
[723] The method of any one of embodiments 716-722, wherein the treatment results in at least one undesirable side effect and wherein the undesirable side effect is reduced relative to administering a plurality of rAAV virions produced from a triple transfection method.
[724] The method of any one of embodiments 716-723, wherein a concentration of rAAV virion neutralizing antibody in the blood serum of the patient is reduced relative to a concentration of rAAV virion neutralizing antibody in the blood serum of a patient after administering a plurality of rAAV virions produced from a triple transfection method.
[725] The method of embodiment 724, wherein the concentration of rAAV virion neutralizing antibodies is measured by an ELISA assay.
[726] A method of administering a dose of rAAV virions having a predetermined number of viral genomes (VG) to a subject with reduced number or intensity of adverse effects as compared to administration of the same rAAV VG dose prepared by transient triple transfection, the method comprising:
   administering a dose of rAAV produced in the cell of any one of the preceding embodiments, the population of cells of any one of the preceding embodiments, or the stable cells of any one of the preceding embodiments.
[727] The method of embodiment 726, wherein the adverse effect is selected from the group consisting of: liver dysfunction, liver inflammation, gastrointestinal infection, vomiting, bacterial infection, sepsis, increases in troponin levels, decreases in red blood cell counts, decreases in platelet counts, activation of the complement immune system response, acute kidney injury, cardio-pulmonary insufficiency, and death.
[728] The method of any one of embodiments 726-727, wherein the adverse effect is an increase in serum levels of one or more of interferon gamma (IFNγ), interleukin 10 (IL-1β), and interleukin 6 (IL-6).
[729] A method of treating a condition or disorder, the method comprising administering a first therapeutically effective amount of the pharmaceutical composition of any one of the preceding embodiments having a predetermined number of viral genomes to a patient in need thereof and a second therapeutically effective amount of the pharmaceutical composition of any one of the preceding embodiments having the predetermined number of viral genomes to the patient in need thereof.
[730] The method of embodiment 729, wherein the first therapeutically effective amount and the second therapeutically effective amount vary by no more than 1%, 5%, 10%, or 15%.
[731] An rAAV virion made by the methods of any one of the preceding embodiments.
[732] A composition comprising a plurality of rAAV virions produced by the method of any one of the preceding embodiments.
[733] An rAAV virion made by the methods of any one of the preceding embodiments.
[734] A composition comprising a plurality of rAAV virions produced by the method of any one of the preceding embodiments.

### 6.18. Examples

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature.

### 6.18.1. Example 1—Mammalian Cell Line with Three Stably Integrated Plasmids

A stable mammalian cell line capable of inducible expression of rAAV encapsidating a payload is constructed by integrating three nucleic acid constructs into the nuclear genome of a cell line that expresses adenovirus E1A and E1B (FIGS. 1, 5B, and 6). As described in FIG. 6, the cell line is a DHFR null HEK293 cell line. Cells that successfully integrated all 3 constructs are selected and maintained by growth in media lacking hypoxanthine and thymidine, and in the presence of blasticidin. Successful integration of construct 1 with concurrent repression of Cre expression from construct 2 is determined by confirming fluorescence emission from EGFP prior to triggering.

### 6.18.2. Example 2—Cre-Inducible Expression of Rep/Cap Proteins

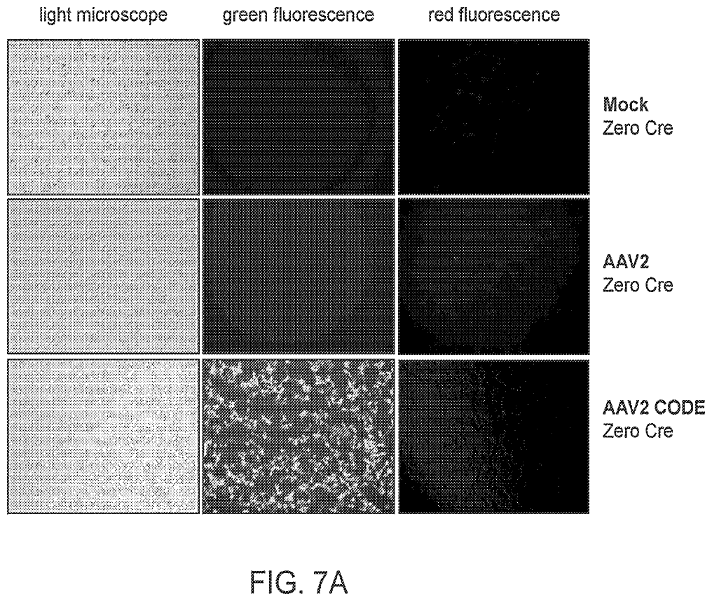

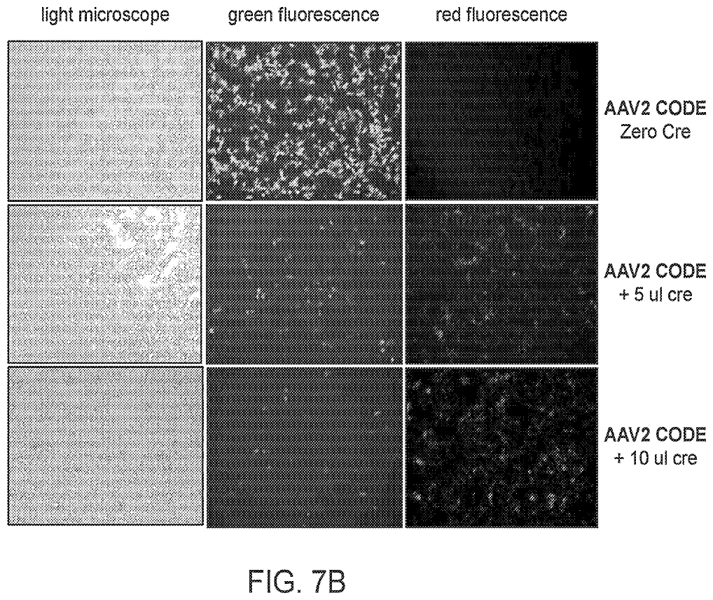

Experiments were performed to test performance of construct 1, as illustrated in FIG. 3A. The construct is designed so that Cre-mediated recombination excises the second spacer segment (FIG. 3B). Excision of the second spacer segment (i) abolishes EGFP expression (FIGS. 7A-7B), (ii) results in production of Rep transcripts from the endogenous P5 and P19 promoters, and (iii) facilitates production of Cap transcripts from an endogenous P40 promoter. The Cap genes are cloned downstream of the Rep gene and operatively linked to the endogenous P40 promoter. Thus, Rep protein expression facilitates Cap protein expression.

HEK293 cells were plated in 24 well plates. Plates were centrifuged for 30 min. at room temperature followed by incubation at 37° C. for 30 min. Media was replaced with growth media and cells were immediately transfected with either an AAV2 positive control plasmid (AAV2) or an AAV2 construct 1 plasmid (AAV CODE) using TransIT 293 reagent. Mock samples were not transfected. Different volumes of Cre gesicles were added to the wells. No Cre gesicles were added to wells imaged in FIG. 7A. No Cre gesicles, 5 μl of Cre gesicles, or 10 μl of Cre gesicles were added as indicated to the wells imaged in FIG. 7B. Cells were imaged 24 hours post transfection and then harvested for protein analysis.

The results are shown in FIGS. 7A-7B, which are light microscope and fluorescence microscope images. Light microscope images are shown in the leftmost column. Green fluorescence microscopic images are shown in the middle column. Red fluorescence images are shown in the rightmost column.

As shown in FIG. 7A, without addition of Cre, cells transfected with construct 1 show intense EGFP fluorescence caused by expression of the Rep-EGFP fusion transcript, splicing of the intron, and translation (see FIG. 3A). FIG. 7B shows decreased GFP fluorescence upon Cre delivery. Cre is delivered by gesicles loaded with Cre protein and red fluorescent marker protein to track delivery into cells. Delivery of Cre affects recombination, with removal of the EGFP cassette from construct 1.

FIG. 8A shows Western blots illustrating that Cre-mediated excision of the excisable spacer segment induces Rep protein production from post-triggered plasmid construct 1. In addition, the presence of the rabbit beta globin intron does not interfere with Rep protein expression level. FIG. 8B shows GFP expression, viability and density graphs of the cells containing the Rep/Cap construct, and blots illustrating Rep production and total protein production.

### 6.18.3. Example 3—Exemplary Sequence and Construction of Construct 1 (the Rep/Cap Construct)

A plasmid encoding the AAV2 genome (pAV2) from the ATCC was utilized to amplify the AAV2 genome, minus the ITRs. This amplified construct was cloned into a pCR Blunt II Topo vector, forming the backbone for construct 1. The construct 1 intervening spacer and Rep/Cap coding sequences were assembled from gblocks and inserted at nucleotide position 1022 (with reference to WT AAV2). This cloning position is downstream from the P19 promoter, away from any known cis regulatory elements. The EGFP cassette in the second excisable spacer element is in frame with proteins produced by both the P5 and P19 transcripts. The details of pre-triggered construct 1 are shown in FIG. 3A, with an exemplary sequence provided in SEQ ID NO: 6, with the accompanying feature descriptions shown below.

### 6.18.4. Example 4—Modifying or Inhibiting Antiviral Responses

Viral proteins needed for AAV virion formation are inhibited by host cell mechanisms. Inhibition of these host cell mechanisms to maximize AAV viral titers in the stable cell lines described herein include, but are not limited to: knocking out PKR (PKR KO) (which is a pathway responsible for inhibition of viral proteins) in the starting cell line (P0), introducing a mutant EIF2alpha (in the PKR pathway) in the starting cell line (P0), and/or manipulating or modulating VA RNA (an inhibitor of PKR). As such, development of three strategies: manipulation of VA RNA, PKR KO, and EIF2alpha mutation, are being developed for use in any combination in the AAV production systems described herein. All three of these strategies can be done in any combination.

### A. Modification of VA RNA for Optimized Expression

VA RNA expression is being analyzed in four ways:
1. Constitutive expression of VA RNA (traditional approach) utilized as a control, no manipulation of the VA RNA promoter or sequence.
2. Inducible VA RNA

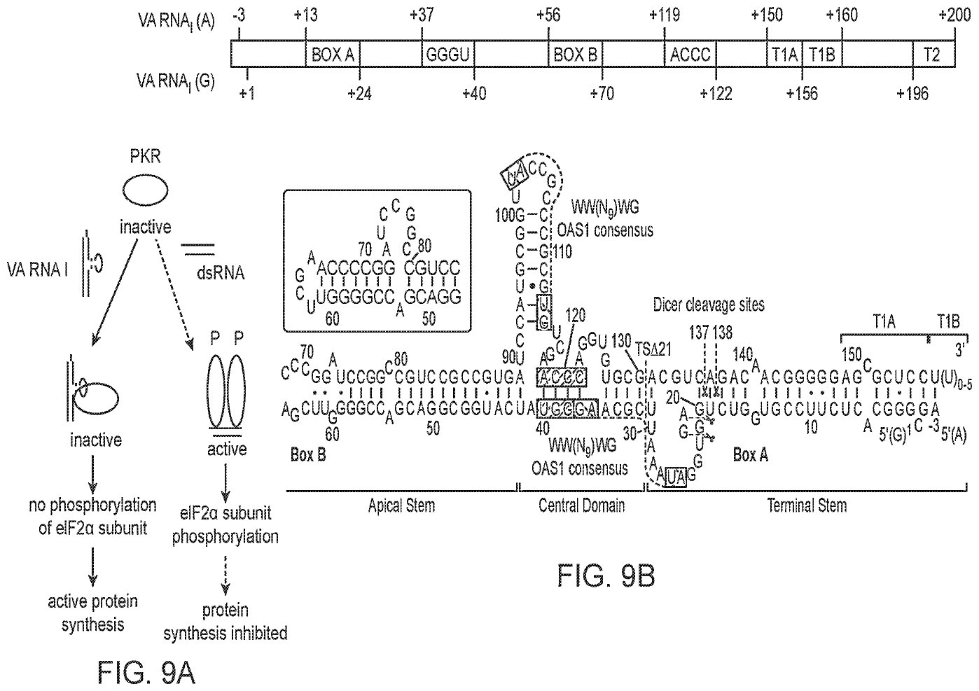

VA RNA naturally has internal constitutive promoters (A Box and B Box; see construct map in upper right hand of FIG. 9B). For experiments to create an inducible VA RNA construct, mutations are first introduced in the internal promoters to abolish their activity.

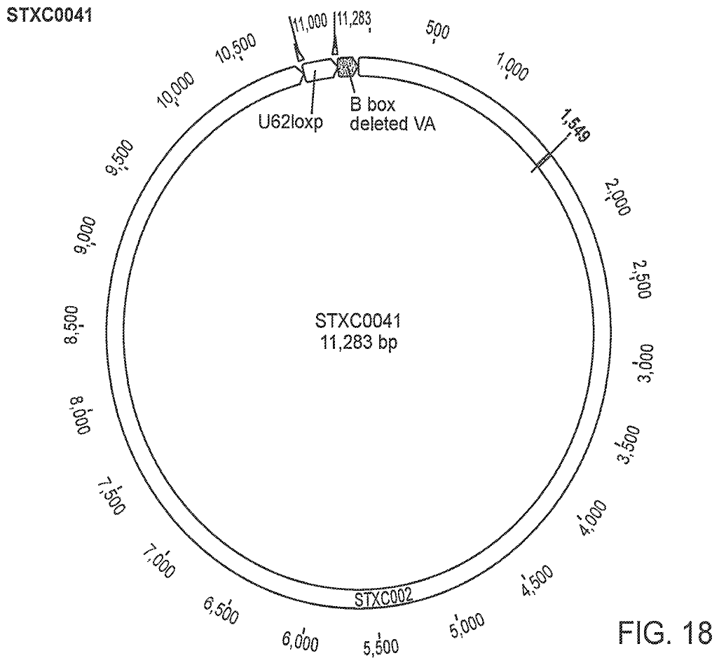

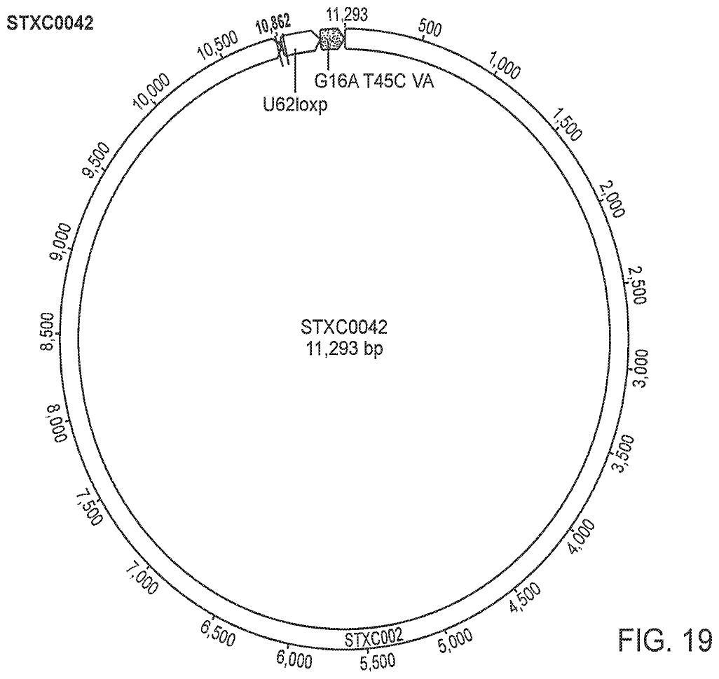

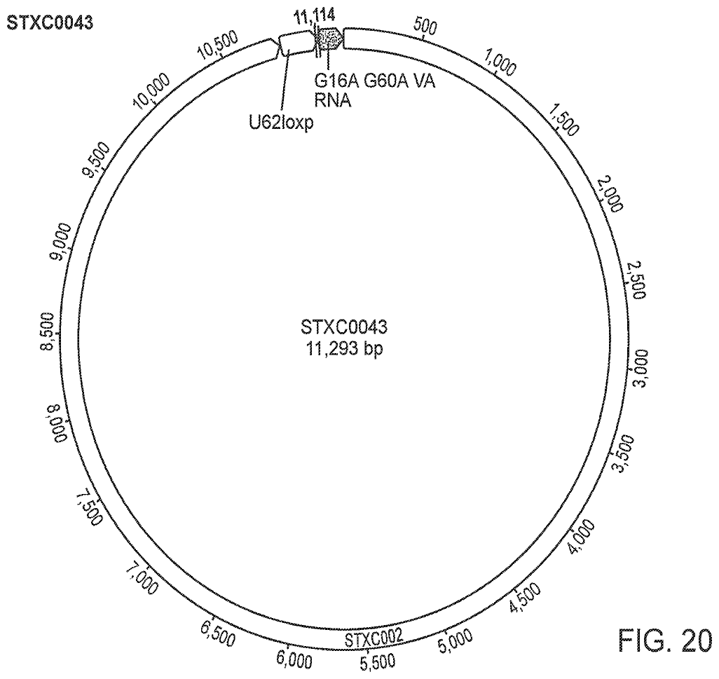

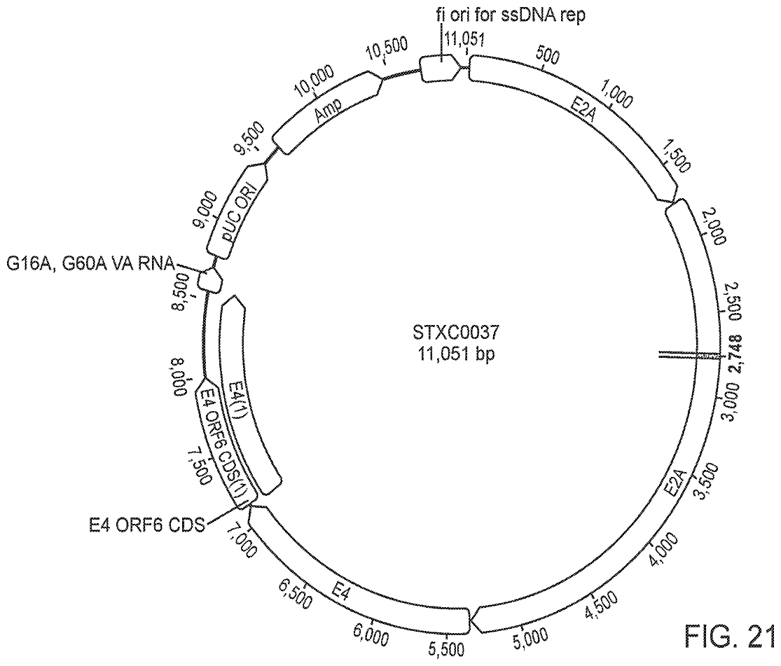

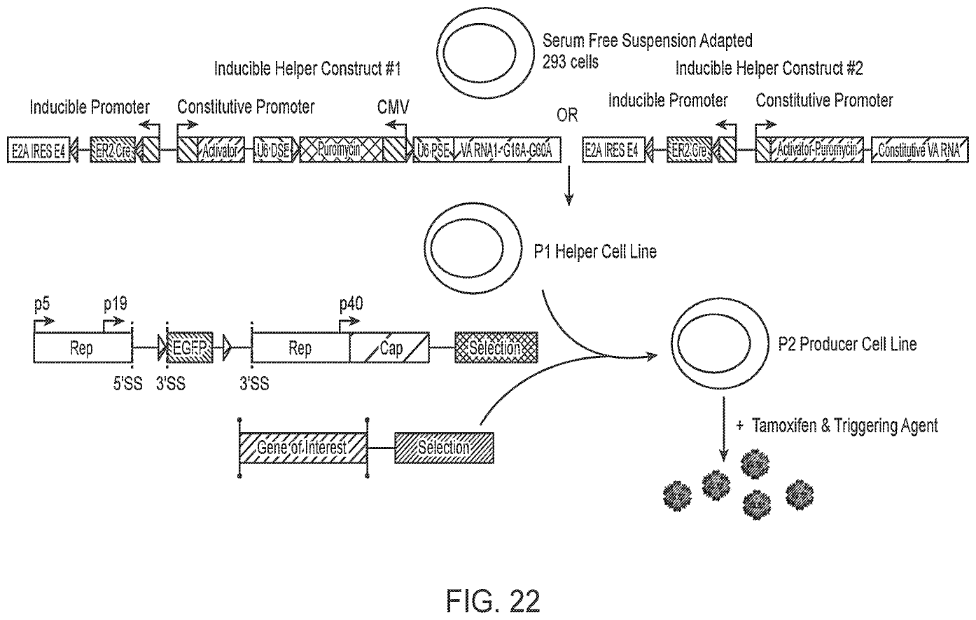

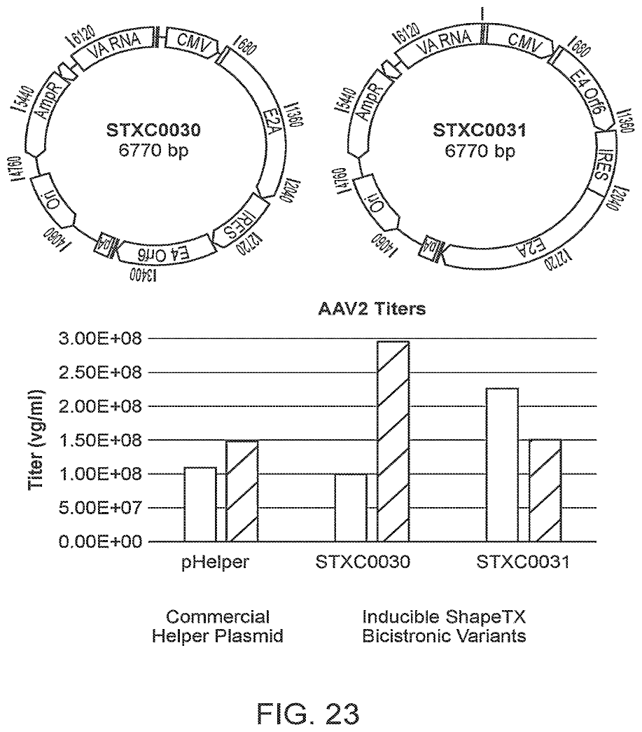

An inducible U6 promoter system is used to drive expression of VA RNA, as shown in FIG. 2C and in FIGS. 18-20. An advantage to this strategy is that better cell viability and higher AAV titers is expected when using an inducible VA RNA system, in which there is an optimized amount of VA RNA in the production cell system since there may be some cell toxicity associated with constitutive expression of VA RNA.
3. No VA RNA+compensatory/analog viral proteins

A third option is being developed where VA RNA is eliminated altogether from the system, and another viral protein (e.g. IC34.5 from HSV; an analog of VA RNA) is tested to replace VA RNA function (inhibition of PKR) in order to determine whether the analog can compensate and/or improve ultimate AAV titers as compared to VA RNA in the production system.

### 4. No VA RNA

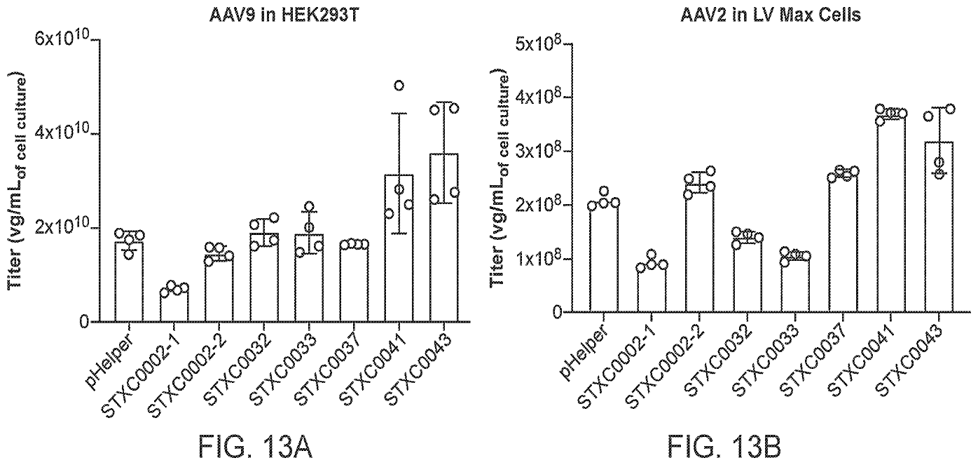

In the scenario that viral protein synthesis is unaffected and there is no real hit to AAV titer in helper constructs where VA RNA is excluded altogether, VA RNA is removed from the production cell system. This would only be feasible in cell lines that have been engineered to be optimal for AAV production (e.g., the LV max cells), See FIG. 13.

The following experiments have all been run via triple transfection. It is expected that the results from the triple transfection experiments can be applied, optimized, and tested in the stable cell line context (e.g., the constructs described herein).

### B: Testing Alternative Viral Proteins to Compensate for VA RNA

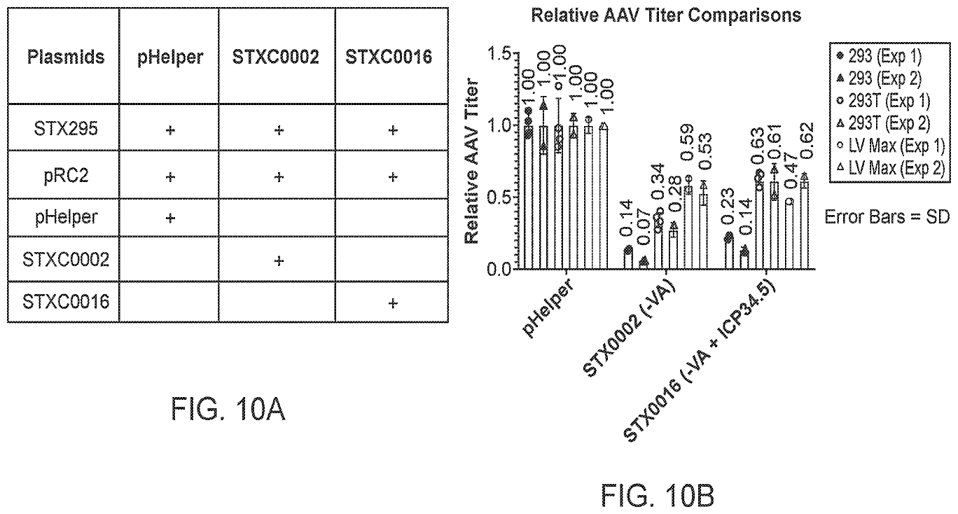

An experiment was run in which VA RNA was substituted with another viral protein (infected cell protein 34.5 (ICP34.5)) to see if AAV titers could be improved. As shown in FIG. 10A, three groups were tested:
pHelper (no change to helper plasmid and VA RNA is present)

This is the positive control for VA RNA, and for these experiments a triple transfection with the following plasmids was performed: the pHelper vector, along with STX295 (construct 3 in which the payload is a fluorescent marker flanked by AAV2 ITRs), and pRC2 (this is the Rep/Cap construct, Rep/ITRs from AAV2 and Cap from AAV2).
STXC0002 (VA RNA is deleted)

This is the negative control for VA RNA and for these experiments, a triple transfection with the following plasmids was performed: the STXC0002 helper vector, along with plasmid STX295 (construct 3 in which the payload is a fluorescent marker flanked by AAV2 ITRs), and pRC2 (this is the Rep/Cap construct, Rep/ITRs from AAV2 and Cap from AAV2).
STXC0016 (VA RNA is deleted and IC34.5 added)

This is an experimental group and a triple transfection with the following plasmids was performed: the STXC0016 helper vector (VA RNA is deleted and IC34.5 added as encoded on the 0016 plasmid), STX295 (construct 3 in which the payload is a fluorescent marker flanked by AAV2 ITRs), and pRC2 (this is the Rep/Cap construct, Rep/ITRs from AAV2 and Cap from AAV2).

### Results: Alternative Viral Proteins to Compensate for VA RNA

FIG. 10B shows the relative AAV titers from triple transfection with each of the three groups from FIG. 10A in 293 cells, 293T cells, and LV max cells. AAV titers were determined by qPCR. AAV titers were relatively abrogated after triple transfection with STXC0002 (VA RNA is deleted). In 293T cells, AAV titers were restored after triple transfection with STXC0016 (VA RNA is deleted and IC34.5 added). In LV max cells, AAV titer was similar between STXC0002 (VA RNA is deleted) and STXC0016 (VA RNA is deleted and IC34.5 added) (FIG. 10B). The results for wildtype VA RNA are shown for the pHelper construct, which serves as the positive control.

### C. Testing Modified or Inducible VA RNA Constructs

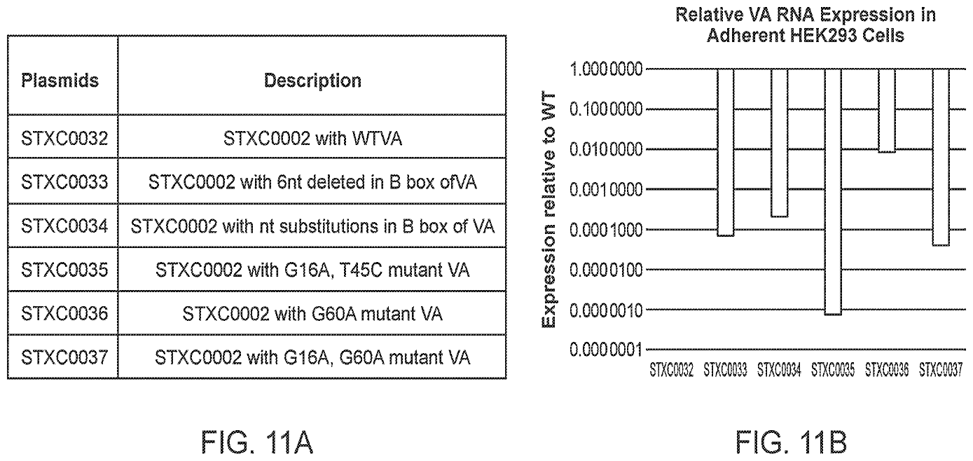

To test modified VA RNA constructs, deletions or mutations were made to the internal promoters in VA-RNA, including deletions or mutations to the A box and the B box. FIG. 11A is a description of each plasmid tested and the corresponding deletions or mutations in VA-RNA. G16A is a mutation in the A box and G60A is a mutation in the B box promoter region. FIG. 11B shows expression of VA RNA relative to the positive control (STXC0032; which is the STXC0002 with WT VA added).

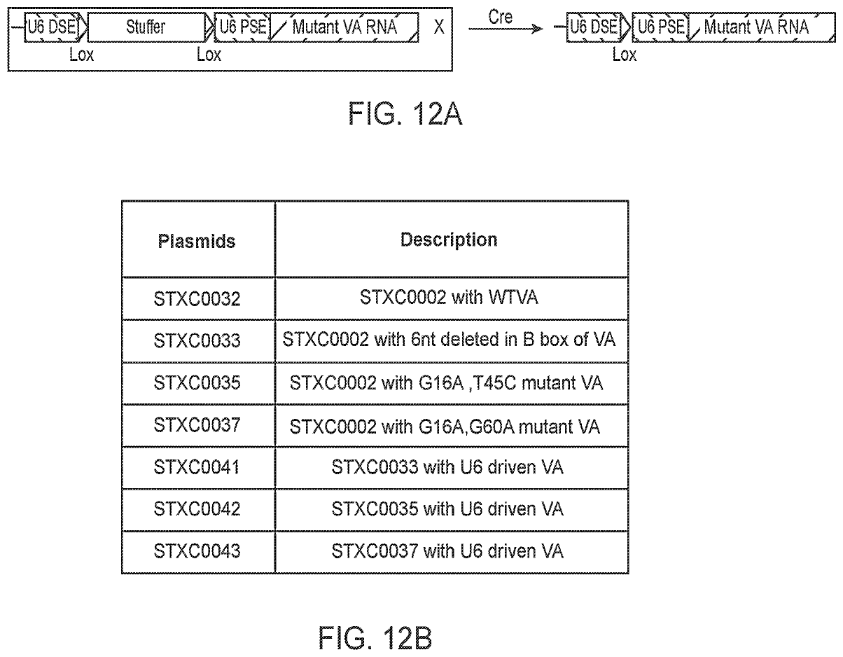

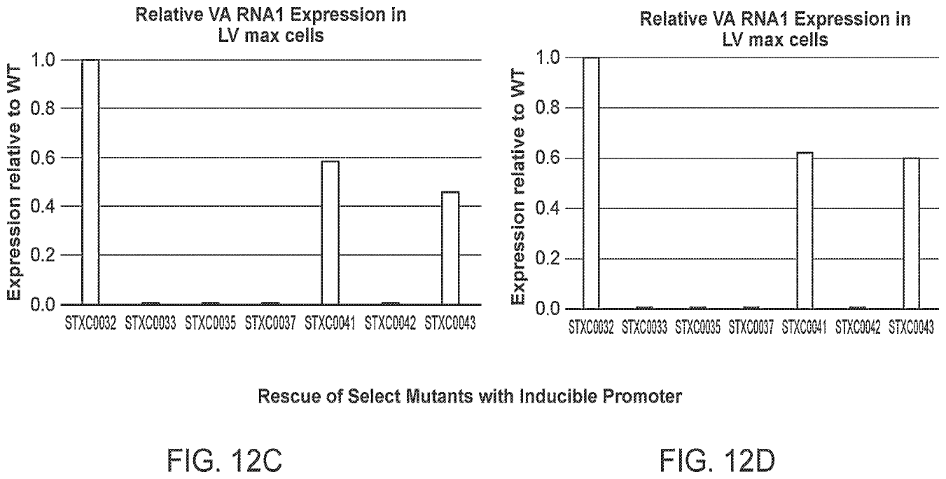

To test an inducible VA RNA system, constructs containing a Cre-inducible U6 promoter were made to drive expression of VA-RNA for each of the mutant VA-RNA constructs shown in FIG. 11A. FIG. 12A shows a schematic of the inducible U6 promoter (a similar schematic is shown in FIG. 2C). In this example, the U6 promoter is separated by a stuffer sequence (PGK-neo), which is flanked by Lox sites. Cre, when present, excises the stuffer sequence thereby mediating recombination and resulting in an inducible U6 promoter. STXC0033, STXC0035, and STXC0037 constructs from FIG. 11A, were modified to include the Cre-inducible U6 promoter, yielding STXC0041, STXC0042, and STXC0043, respectively (FIG. 12B).

### Results: Inducible VA RNA Constructs

While STXC41 and STXC043 displayed similar levels of relative VA RNA expression, STXC343 achieved these levels of VA RNA expression with less disruption to VA RNA (with just the two mutations: G16A and G60A) as compared to the STXC0041 plasmid (which has a 6 nucleotide deletion in the B box promoter region) (FIG. 12C-12D).

FIGS. 13A-13B show results from a triple transfection experiment which was carried out for a subset of the plasmid constructs from FIG. 12B, using AAV9 in HEK293T cells (FIG. 13A) and AAV2 in LV Max cells (FIG. 13B). Triple transfections utilizing the STXC0041 and STXC0043 constructs displayed the highest levels of titers.

The tables below show sequences of various elements of the above constructs.

| Construct | Sequence of VA RNA |
|---|---|
| STXC0032 | GGGCACTCTTCCGTGGTCTGGTGGATAAAT |
| SEQ ID | TCGCAAGGGTATCATGGCGGACGACCGGGG |
| NO: 13 | TTCGAACCCCGGATCCGGCCGTCCGCCGTG |
| | ATCCATGCGGTTACCGCCCGCGTGTCGAAC |
| | CCAGGTGTGCGACGTCAGACAACGGGGGAG |
| | CGCTCCTTTTT |
| STXC0033 | GGGCACTCTTCCGTGGTCTGGTGGATAAAT |
| SEQ ID | TCGCAAGGGTATCATGGCGGACGACCGGCC |
| NO: 14 | GGATCCGGCCGTCCGCCGTGATCCATGCGG |
| | TTACCGCCCGCGTGTCGAACCCAGGTGTGC |
| | GACGTCAGACAACGGGGGAGCGCTCCTTTT |
| | T |
| STXC0035 | GGGCACTCTTCCGTGATCTGGTGGATAAAT |
| SEQ ID | TCGCAAGGGTATCATGGCGGACGACCGGGG |
| NO: 15 | TTCGAACCCCGGATCCGGCCGTCCGCCGTG |
| | ATCCATGCGGTTACCGCCCGCGTGTCGAAC |
| | CCAGGTGTGCGACGTCAGACAACGGGGGAG |
| | CGCTCCTTTTT |
| STXC0037 | GGGCACTCTTCCGTGATCTGGTGGATAAAT |
| SEQ ID | TCGCAAGGGTATCATGGCGGACGACCGGGA |
| NO: 16 | TTCGAACCCCGGATCCGGCCGTCCGCCGTG |
| | ATCCATGCGGTTACCGCCCGCGTGTCGAAC |
| | CCAGGTGTGCGACGTCAGACAACGGGGGAG |
| | CGCTCCTTTTT |

| | Sequence of 5′ U6 DSE |
|---|---|
| Construct | to 3′ VA RNA |
| STXC0041 | CGATGGAGGGCCTATTTCCCATGATTCCTT |
| SEQ ID | CATATTTGCATATACGATACAAGGCTGTTA |
| NO: 17 | GAGAGATAATTAGAATTAATTTGACTGTAA |
| | ACACAAAGATATTAGTACAAAATAATAACT |
| | TCGTATAATGTATGCTATACGAAGTTATTT |
| | TGCAGTTTTAAAATTATGTTTTAAAATGGA |
| | CTATCATATGCTTACCGTAACTTGAAAGTA |
| | TTTCGATTTCTTGGCTTTATATATCTTGTG |
| | GAAAGGACGAAACACCGGGCACTCTTCCGT |
| | GGTCTGGTGGATAAATTCGCAAGGGTATCA |
| | TGGCGGACGACCGGCCGGATCCGGCCGTCC |
| | GCCGTGATCCATGCGGTTACCGCCCGCGTG |
| | TCGAACCCAGGTGTGCGACGTCAGACAACG |
| | GGG |
| STXC0042 | CGATGGAGGGCCTATTTCCCATGATTCCTT |
| SEQ ID | CATATTTGCATATACGATACAAGGCTGTTA |
| NO: 18 | GAGAGATAATTAGAATTAATTTGACTGTAA |
| | ACACAAAGATATTAGTACAAAATAATAACT |
| | TCGTATAATGTATGCTATACGAAGTTATTT |
| | TGCAGTTTTAAAATTATGTTTTAAAATGGA |
| | CTATCATATGCTTACCGTAACTTGAAAGTA |
| | TTTCGATTTCTTGGCTTTATATATCTTGTG |
| | GAAAGGACGAAACACCGGGCACTCTTCCGT |
| | GATCTGGTGGATAAATTCGCAAGGGTATCA |
| | TGGCGGACGACCGGGGTTCGAACCCCGGAT |
| | CCGGCCGTCCGCCGTGATCCATGCGGTTAC |
| | CGCCCGCGTGTCGAACCCAGGTGTGCGACG |
| | TCAGACAACGGGGGAGCGCTCCTTTTT |
| STXC0043 | TTCACTAGAATCGATGGAGGGCCTATTTCC |
| SEQ ID | CATGATTCCTTCATATTTGCATATACGATA |
| NO: 19 | CAAGGCTGTTAGAGAGATAATTAGAATTAA |
| | TTTGACTGTAAACACAAAGATATTAGTACA |
| | AAATAATAACTTCGTATAATGTATGCTATA |
| | CGAAGTTATTTTGCAGTTTTAAAATTATGT |
| | TTTAAAATGGACTATCATATGCTTACCGTA |
| | ACTTGAAAGTATTTCGATTTCTTGGCTTTA |
| | TATATCTTGTGGAAAGGACGAAACACCGGG |
| | CACTCTTCCGTGATCTGGTGGATAAATTCG |
| | CAAGGGTATCATGGCGGACGACCGGGATTC |
| | GAACCCCGGATCCGGCCGTCCGCCGTGATC |
| | CATGCGGTTACCGCCCGCGTGTCGAACCCA |
| | GGTGTGCGACGTCAGACAACGGGGGAGCGC |
| | TCCTTTTT |

### 6.18.5. Example 5—Rep/Cap Construct Integration into a Stable Cell Line

This example describes integration of construct one encoding Rep and Cap polypeptides (SEQ ID NO: 7) into a stable cell line and describes inducible expression of Rep and Cap polypeptides in said stable cell lines. An AAV2 genome without the ITRs and with the polynucleotide construct shown at the top right of FIG. 8B was cloned into a piggybac vector with a Blasticidin resistance gene (SEQ ID NO: 8). The excisable element interrupting Rep was inserted downstream of the p19 promoter, as shown in FIG. 8B.

Suspension HEK293 cells (viral production cells, VPCs; also referred to as the parental cells or parental VPC pool) were transfected using a PEI pro transfection reagent. The ratio of transposon to transposase ratio used was 2:1. Cells were allowed to recover in non-selective media for 72 hours and passaged into selective media (10 μg/ml Blasticidin). Cell growth and viability was monitored every 3 to 4 days using a Vicell cell counter. After full recovery, doubling time of the cell pool was around 25 hours, which is comparable to that of the parental VPC pool, indicating that there were no negative effects from the integrated AAV sequences (FIG. 8B, top graph).

Cells were analyzed by flow cytometry to quantify GFP expressing cells. As shown in FIG. 8B (on the left, the bottom FACS plot), almost all cells were GFP positive, thus, confirming successful integration of the rep/cap polynucleotide construct shown in FIG. 8B into the cells.

### 6.18.6. Example 6—Cre Mediated Induction of Rep Proteins

This example describes Cre mediated induction of Rep proteins in a stable cell line integrated with the Rep/Cap construct of FIG. 8B and described in Example 5. VPCs integrated with the AAV2 Rep/Cap construct of FIG. 8B were treated with Cre gesicles. Briefly 200,000 cells were treated with 5 μl of Cre gesicles in 10 μg/ml polybrene media. Cells were centrifuged at 25,000 rpm for 30 minutes and incubated for 2 hrs at 37° C. Parental cells were used as control. After the incubation, cells were resuspended in fresh media and incubated for an additional 24 hours. Rep expression was analyzed by Western blot using an anti-Rep antibody. Results shown in FIG. 8B demonstrates inducible expression of various Rep isoforms upon Cre treatment.

### 6.18.7. Example 7—Inducible Helper Constructs

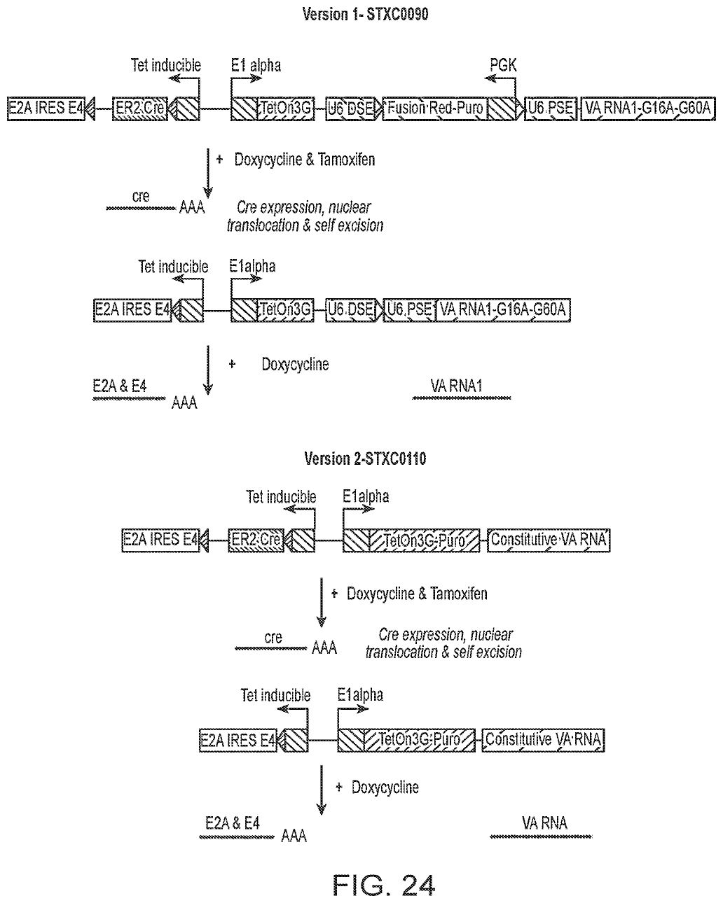

This example describes inducible helper constructs of the present disclosure. Two versions of inducible helper constructs disclosed herein are shown in FIG. 24. A tetracycline/doxycycline (“Dox”) inducible promoter (TRE3G) drives the expression of estrogen inducible cre (ER2 cre). The estrogen inducible cre has a strong polyadenylation signal (stop signal) at its 3′ end. The cre gene and the polyadenylation signal are flanked by lox sites. Following this is a bicistronic E2A E4, orf6 cassette. The plasmid also has a constitutive promoter (mutant EF1a), which drives the expression of Tet-on 3G (Tet responsive activator protein).

Mechanism of action: In the off state (in the absence of Dox), Tet-on 3G is unable to bind the Tet operator elements in the TRE3G promoter and, thus, the TRE3G promoter is not active. In an embodiment of the system, an estrogen responsive Cre is used instead of simple Cre to counteract any basal (or “leaky”) expression of the TRE3G promoter. Thus, even if the system yields leaky expression of the Cre gene, the expressed Cre protein will be held inactive in the cytoplasm. The strong polyadenylation stop signal positioned 3′ of the Cre gene will prevent basal expression of adenoviral helper genes (E2A and E4).

To induce expression, Dox and Tamoxifen are added to the cell culture. Dox binds to the Tet-on 3G protein and promotes binding of Tet-on 3G to the Tet operator elements in the TRE3G promoter. This triggers activation of the promoter. ER2 Cre is expressed at high levels and Tamoxifen brings Cre to the nucleus. Cre recombines the lox sites, causing excision of the Cre-polyadenylation cassette. This brings the bicistronic E2A and E4 cassette next to the Tet inducible promoter triggering their expression. Self-excision of Cre will limit the duration of Cre expression in the cells thus limiting Cre related toxicity and promiscuous recombination events.

A first version of an inducible helper construct is shown in FIG. 24, at left. The construct shown in FIG. 24 (at left) also has a mutant of VA RNA (G16A and G60A, which disable the internal PolIII promoter) driven by a U6 promoter. The proximal sequence element (PSE) and distal sequence element (DSE) of the U6 promoter are separated by a Lox sequence-flanked stuffer sequence (PGK driven Fusion Red-PuroR), thus, disabling the promoter. The promoter is reconstituted by Cre mediated excision of the stuffer sequence, resulting in VA RNA expression conditional upon Cre expression

A second version of an inducible helper construct is shown in FIG. 24, at right. The construct shown in FIG. 24 (at right) has a constitutively expressed VA RNA element driven by its internal native promoters.

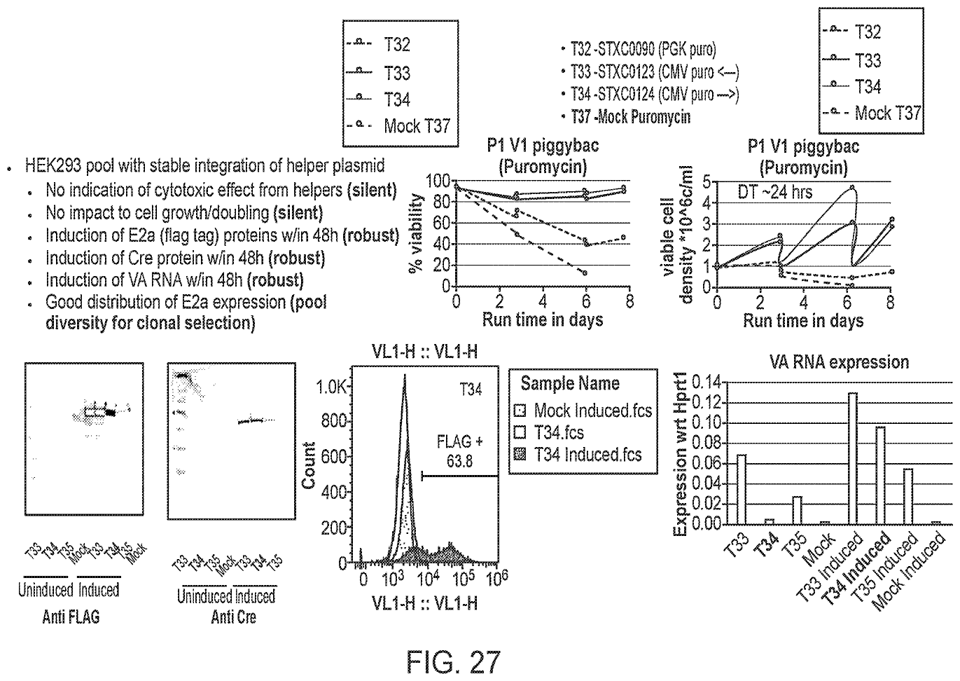

FIG. 25 shows multiple variations of the inducible helper construct shown in FIG. 24 (at left). Fusion Red-PuroR is replaced with PuromycinR and the PGK promoter is replaced with a CMV promoter in two different orientations. Suspension HEK293 cells (viral production cells, VPCs) were transfected with different variations of the inducible helper construct (shown in FIG. 25) encoded for in plasmids using a PEI pro transfection reagent. The ratio of transposon to transposase ratio used was 2:1. Cells were allowed to recover in non-selective media for 72 hrs and passaged into selective media (1.5 μg/ml Puromycin). Cell growth and viability was monitored every 3 to 4 days using a Vicell cell counter. Results are shown in the top graphs of FIG. 27. After full recovery, doubling time of the cell pools was around 23 hours which is comparable to that of parental VPCs, indicating no negative effects of integrated sequences.

### 6.18.8. Example 8—Helper Constructs Stably Integrated into Cell Lines

This example describes stable integration of inducible helper constructs of the present disclosure into cell lines. Pools were induced with 20 ng/ml Dox and 2 uM Tamoxifen and analyzed 48 hours post induction. Pools showed no basal expression and robust expression of E2A post induction by both western blot (FIG. 27) and intracellular staining as detected by anti-FLAG antibody. RNA samples were analyzed by RT QPCR using VA RNA specific primers and probes showing upregulation of VA RNA expression post-induction (FIG. 27). FIG. 27 shows an overview of HEK293 cells with the stably integrated helper plasmid showing no cytotoxic effects and induction of Cre, production of VA RNA and good distribution of E2a expression.

### 6.18.9. Example 9—Inducible Stable Cell Lines

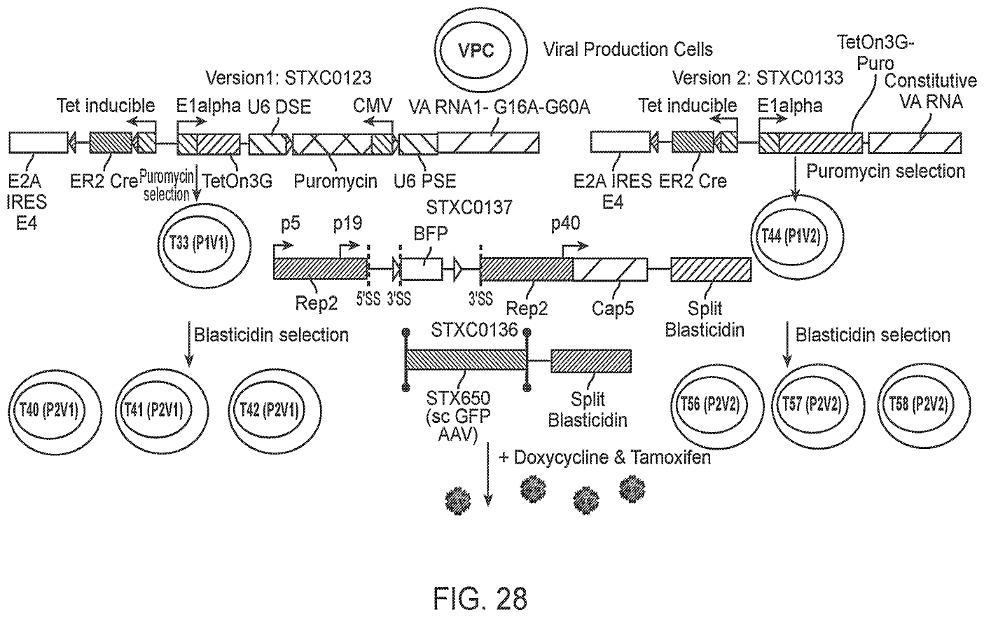

This example describes production of inducible stable cell lines using the constructs of the present disclosure. Two versions of inducible helper constructs (Version 1: STXC0123 and Version 2: STXC0133) disclosed herein are shown in FIG. 28. In both inducible helper constructs, a tetracycline/doxycycline (“Dox”) inducible promoter (TRE3G) drives the expression of estrogen inducible Cre (ER2 Cre). The estrogen inducible Cre has a strong polyadenylation signal (stop signal) at its 3′ end. The Cre gene and the polyadenylation signal are flanked by lox sites. Following this is a bicistronic E2A E4, orf6 cassette. The plasmid also has a constitutive promoter (mutant EF1a), which drives the expression of Tet-on 3G (Tet responsive activator protein).

However, STXC0123 comprises a mutant of VA RNA (G16A and G60A, which disable the internal PolIII promoter) driven by a U6 promoter. The proximal sequence element (PSE) and distal sequence element (DSE) of the U6 promoter were separated by a Lox sequence-flanked stuffer sequence (CMV driven Puromycin resistance gene), thus, disabling the promoter. The promoter is reconstituted by Cre mediated excision of the stuffer sequence, resulting in VA RNA expression conditional upon Cre expression

In contrast, STXC0 133 comprises a TetOn-3G puromycin resistance gene cassette and a constitutively expressed VA RNA element driven by its internal native promoters.

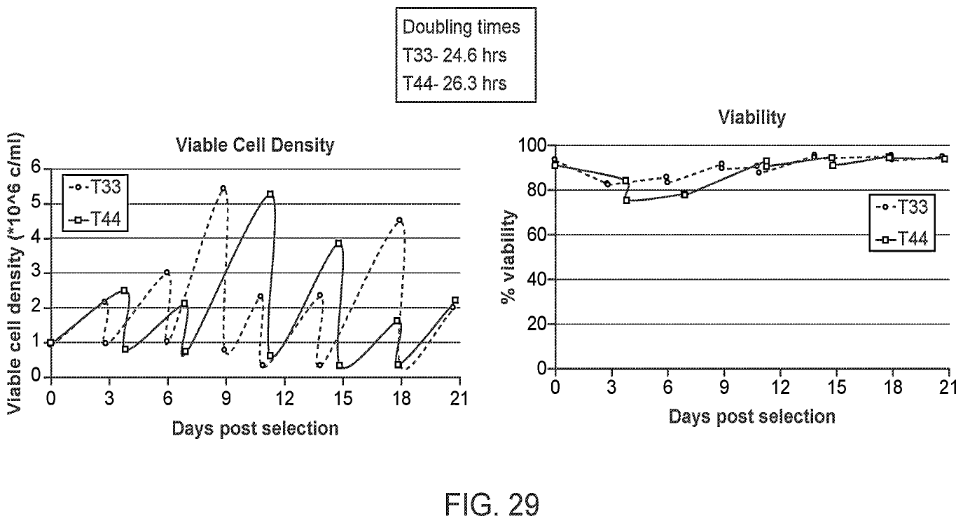

Suspension HEK293 cells (viral production cells, VPCs) were transfected with STXC0123 or STXC0 133 encoded for in plasmids using a transfection reagent. Cells were allowed to recover in non-selective media and passaged into media comprising Puromycin. For both versions, puromycin selection was used to ensure construct integration into the viral production cells (VPCs). STXC0123 produced the T33 (P1V1) cell line and STC0133 produced the T44 (P1V2) cell line. Viable cell density and viability was monitored every 3 days for T33 and T44. Results are shown in the graphs of FIG. 29. After full recovery, doubling time of the cell pools was around 24.6 hours for T33 and 26.3 hours for T44 hours, which is comparable to that of parental VPCs, indicating no negative effects of integrated sequences.

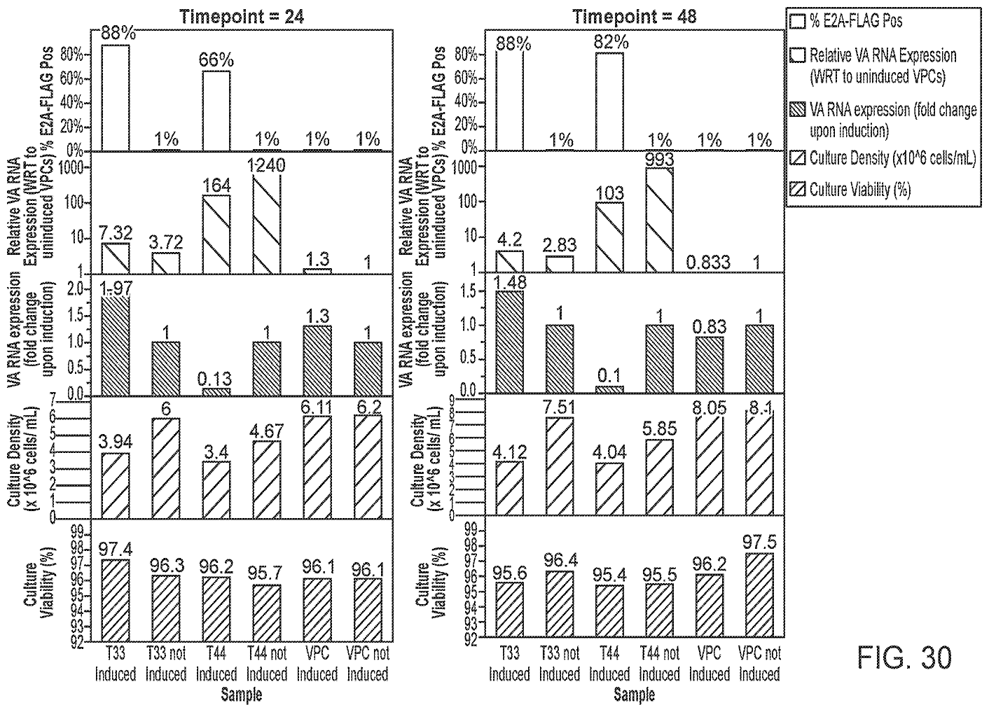

To confirm the stable integration and inducibility of T33 and T44 pools, cells from these pools were tested for E2A expression, VA RNA expression, culture density, and cell viability. Cells from the T33 pool and T44 pool were seeded at 1 million cells/mL, induced with 160 ng/ul Dox and 4 uM Tamoxifen, and analyzed at 24 hours and 48 hours post induction. Pools showed minimal basal expression and robust expression of E2A post induction at both 24 hours and 48 hours (top graphs in FIG. 30). RNA samples were analyzed by RT QPCR using VA RNA specific primers and probes showing VA RNA expression relative to uninduced VPCs at 24 hours and 48 hours post-induction (second graphs from top in FIG. 30) or showing VA RNA expression compared to uninduced T33 or T44 cells at 24 hours and 48 hours post-induction (third graphs from top in FIG. 30). Culture density (fourth graphs from top in FIG. 30) and cell viability (bottom graphs in FIG. 30) also tested at 24 hours and 48 hours post-induction.

Next, the Rep/Cap construct (STXC0137) and payload construct (STC0136) were transfected using a transfection reagent into the T33 (P1V1) cell line and the T44 (P1V2) cell line for integration. For both T33 and T44, the Rep/Cap construct was designed to permit constitutive expression of one half of a split blasticidin resistance gene and expression of AAV Rep and Cap proteins from their endogenous promoters after induction. The construct shown in FIG. 28 is pre-induction of the integrated nucleic acid construct. An intervening spacer interrupts the Rep coding sequence. The intervening spacer comprises a first spacer segment, a second spacer segment which is excisable (BFP flanked by Lox sites), and a third spacer segment. The transcript contains a single intron flanked by 5′ and 3′ splice sites.

The payload construct (STXC0136) comprises a sequence that encodes GFP flanked by ITRs (STX650 (sc GFP AAV)) and the other half of a split blasticidin resistance gene, both under control of a constitutive promoter.

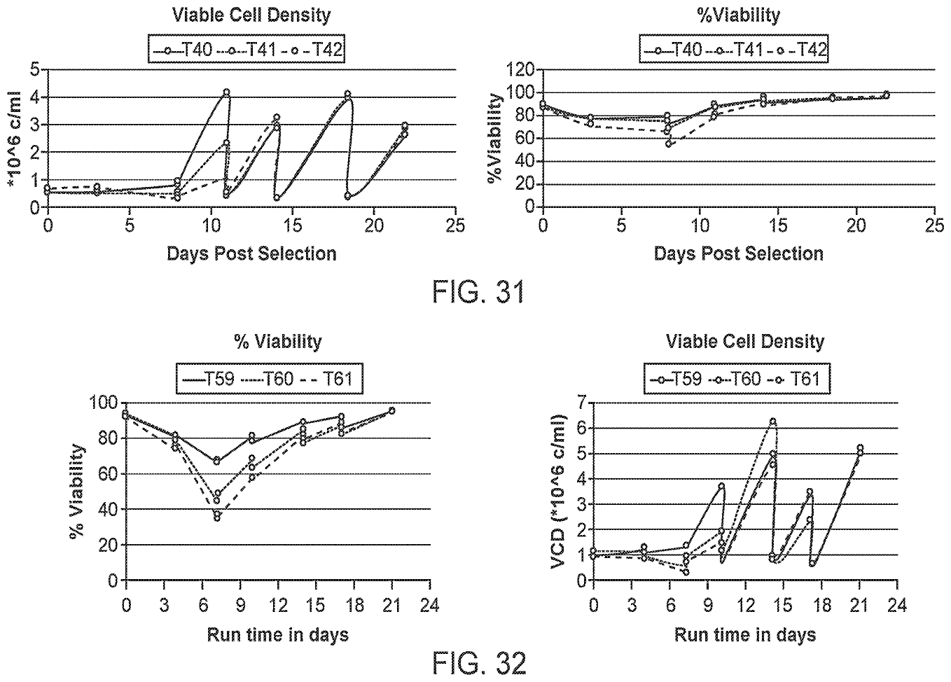

After transfection, cells were allowed to recover in non-selective media and then were passaged into media comprising Blasticidin. For both versions, Blasticidin selection was used to ensure both constructs integrated into the T33 stable cell line and T44 stable cell line. The integration in T33 produced three stable cell lines: T40 (P2V1), T41 (P2V1), and T42 (P2V1). The integration in T44 produced three stable cell lines: T56 (P2V2), T57 (P2V2), and T58 (P2V2). Viable cell density and viability was monitored for T40, T41, and T42 (FIG. 31), and for T59, T60, and T62 (FIG. 32).

In the off state (in the absence of Dox), Tet-on 3G is unable to bind the Tet operator elements in the TRE3G promoter and, thus, the TRE3G promoter is not active. In the constructs of FIG. 28, an estrogen responsive Cre was used instead of simple Cre to counteract any basal (or “leaky”) expression of the TRE3G promoter. Thus, even if the system yields leaky expression of the Cre gene, the expressed Cre protein will be held inactive in the cytoplasm. The strong polyadenylation stop signal positioned 3′ of the Cre gene will prevent basal expression of adenoviral helper genes (E2A and E4).

To induce expression, Dox and Tamoxifen are added to the cell culture of the T40, T41, T42, T56, T57, and T58 stable cells lines. Dox bound to the Tet-on 3G protein and promoted binding of Tet-on 3G to the Tet operator elements in the TRE3G promoter. This triggered activation of the promoter. ER2 Cre was expressed at high levels and Tamoxifen brought Cre to the nucleus. Cre recombined the lox sites, causing excision of the Cre-polyadenylation cassette. This brings the bicistronic E2A and E4 cassette next to the Tet inducible promoter triggering their expression. Cre additionally excised the puromycin resistant gene cassette of of STXC0123, reconstituting the U6 promoter and subsequently allowing expression of the mutant VA RNA1 G16A and G60A. Cre also excised the second spacer segment of STX0137, which includes the BFP marker coding sequence and the upstream 3′ splice site. As rearranged, the STX0137 construct allowed expression of functional Rep and Cap transcripts from their respective endogenous promoters. Self-excision of Cre limited the duration of Cre expression in the cells thus limiting Cre related toxicity and promiscuous recombination events. Induction of the helper constructs and Rep/Cap constructs subsequently allowed capsid production and packaging of the STXC0136 in the produced capsids.

### 6.18.10. Example 10—Induction of Inducible Stable Cell Lines

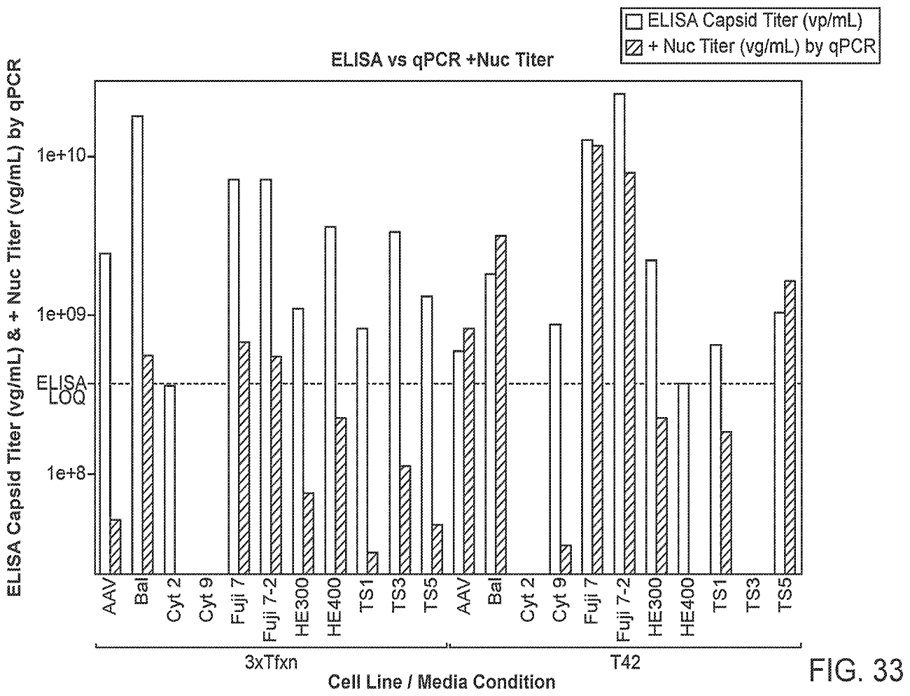

This example describes induction of the inducible stable cell lines of the present disclosure. Cells from the T42 pool cell line (T42) and cells from triple transfection of VPCs (3×Tfxn) were induced in different cell medias. Capsid ELISA was performed to determine total capsid titer. Nuclease treatment and qPCR were performed to determine the titer of capsids encapsidating the viral genome (e.g., the payload construct). FIG. 33 shows a graph of capsid production in different cell media from the T42 pool stable cell line after induction compared to capsid production in cells after triple transfection. The left bar for each media type indicates total capsid titer and the right bar for each media type indicates the titer of capsids encapsidating a viral genome (e.g., the payload construct). The total capsid titer as shown by ELISA was higher for the T42 cells induced in Fuji 7, Fuji 7-2, and HE 300 media compared to the cells produced by triple transfection. The below Table 1 shows the total capsid titer and the titer of capsids encapsidating a viral genome of FIG. 33.

TABLE 1
Total capsid titer and the titer of capsids encapsidating
a viral genome of T42 cells versus 3xTfxn cells
after induction in different cell medias.

| Cell | | Total Capsid | Viral Genome Encapsidated |
|---|---|---|---|
| Line | Media | Titer (vp/mL) | Titer (vg/mL) |
| T42 | Fuji 7 | 1.29 × 10¹⁰ | 1.16 × 10¹⁰ |
| T42 | Fuji 7-2 | 2.48 × 10¹⁰ | 7.92 × 10⁹ |
| T42 | Bal | 1.81 × 10⁹ | 3.18 × 10⁹ |
| T42 | TS5 | 1.03 × 10⁹ | 1.64 × 10⁹ |
| T42 | AAV | 5.95 × 10⁸ | 8.30 × 10⁸ |
| T42 | HE300 | 2.24 × 10⁹ | 2.26 × 10⁸ |
| T42 | TS1 | 6.49 × 10⁸ | 1.86 × 10⁸ |
| T42 | Cyt9 | 8.69 × 10⁸ | 3.52 × 10⁷ |
| T42 | HE400 | 3.74 × 10⁸ | |
| T42 | Cyt2 | | |
| T42 | TS3 | | |
| 3xTfxn | Fuji 7 | 7.21 × 10⁹ | 6.83 × 10⁸ |
| 3xTfxn | Fuji 7-2 | 1.79 × 10¹⁰ | 5.59 × 10⁸ |
| 3xTfxn | Bal | 7.18 × 10⁹ | 5.45 × 10⁸ |
| 3xTfxn | TS5 | 3.62 × 10⁹ | 2.26 × 10⁸ |
| 3xTfxn | AAV | 3.35 × 10⁹ | 1.12 × 10⁸ |
| 3xTfxn | HE300 | 1.09 × 10⁹ | 7.63 × 10⁷ |
| 3xTfxn | TS1 | 2.44 × 10⁹ | 5.20 × 10⁷ |
| 3xTfxn | Cyt9 | 1.31 × 10⁹ | 4.77 × 10⁷ |
| 3xTfxn | HE400 | 8.27 × 10⁸ | 3.20 × 10⁷ |
| 3xTfxn | Cyt2 | 3.60 × 10⁸ | BLOQ |
| 3xTfxn | TS3 | BLOQ | BLOQ |

*BLOQ = below assay limit of quantitation

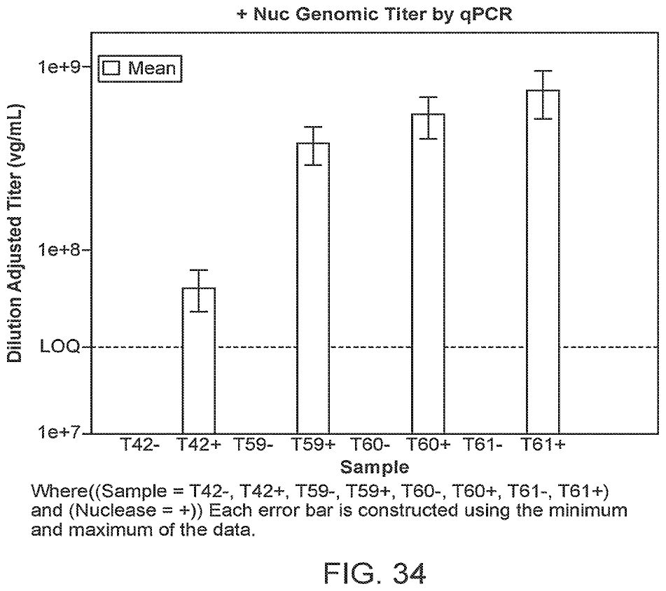

Cells from the T42 stable cell line pool, the T59 stable cell line pool, the T60 stable cell line pool, or the T61 stable cell line pool were either not induced (−) or induced (+) in HE300 media. Nuclease treatment and qPCR were performed to determine the titer of capsids encapsidating the viral genome (e.g., the payload construct) (FIG. 34).

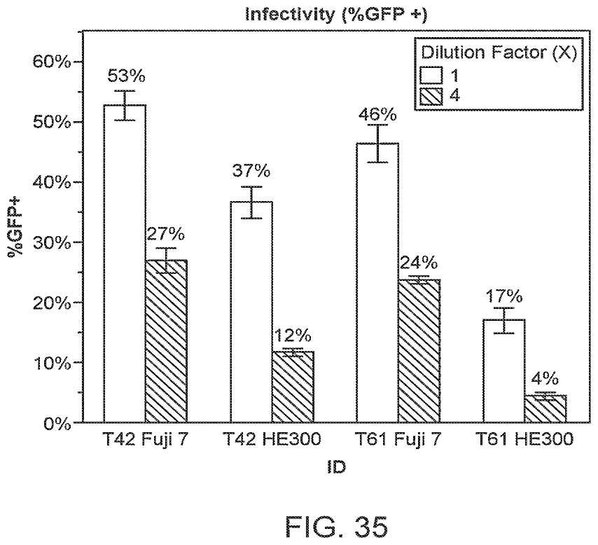

Capsids from induction of the T42 pool stable cell line and capsids from induction of the T61 pool stable cell line were used to infect cells and determine infectivity. The percentage of GFP+ cells after infecting cells with capsids (payload was GFP) is shown in FIG. 35. The left bar for each cell line type/media is for a dilution factor of 1 and the right bar for each cell line type/media is for a dilution factor of 4.

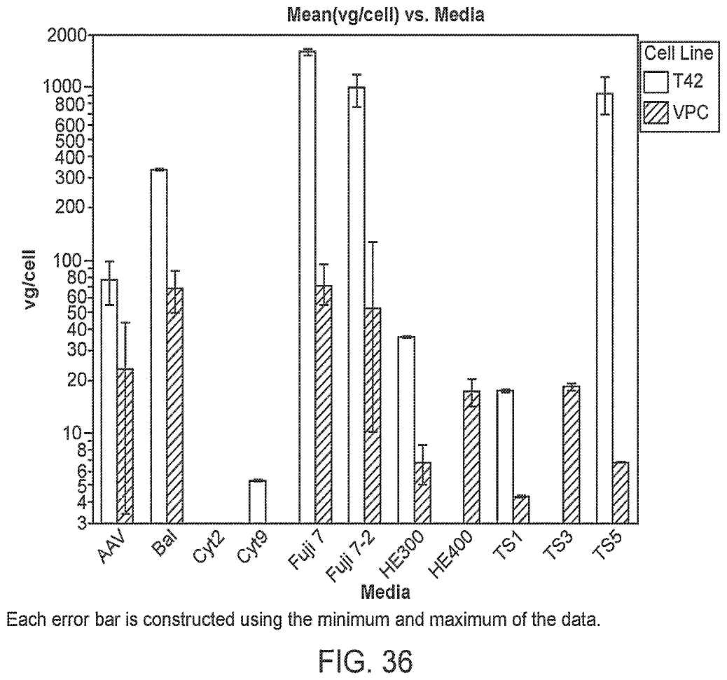

The virus produced per cell (titer productivity) was also tested for the T42 pool stable cell line after induction compared to the triple transfected parental cells (VPC). Cultures of T42 stable cell line were induced at 3×10⁶ cells/mL and harvested 96 hours post induction. Cultures of the parental cell line (VPCs) were triple transfected at 3×10⁶ cells/mL and harvested 96 hours post transfection. Harvested cells underwent nuclease treatment (benzonase) and qPCR was performed to determine the titer of capsids encapsidating the viral genome (e.g., the payload construct). Titer productivity (vg/cell) on a per cell basis was calculated by dividing the total qPCR titer by the viable cell density at harvest. FIG. 36 shows a graph of capsids encapsidating a viral genome (e.g., the payload construct) in different cell media from the T42 pool stable cell line after induction compared to the triple transfected parental cells (VPC). The left bar for each media type indicates titer of capsids encapsidating a viral genome produced per cell by cells from the T42 pool stable cell line and the right bar for each media type indicates titer of capsids encapsidating a viral genome produced per cell by cells from the triple transfected parental cell line (VPC).

### 6.18.11. Example 11—Media Screen

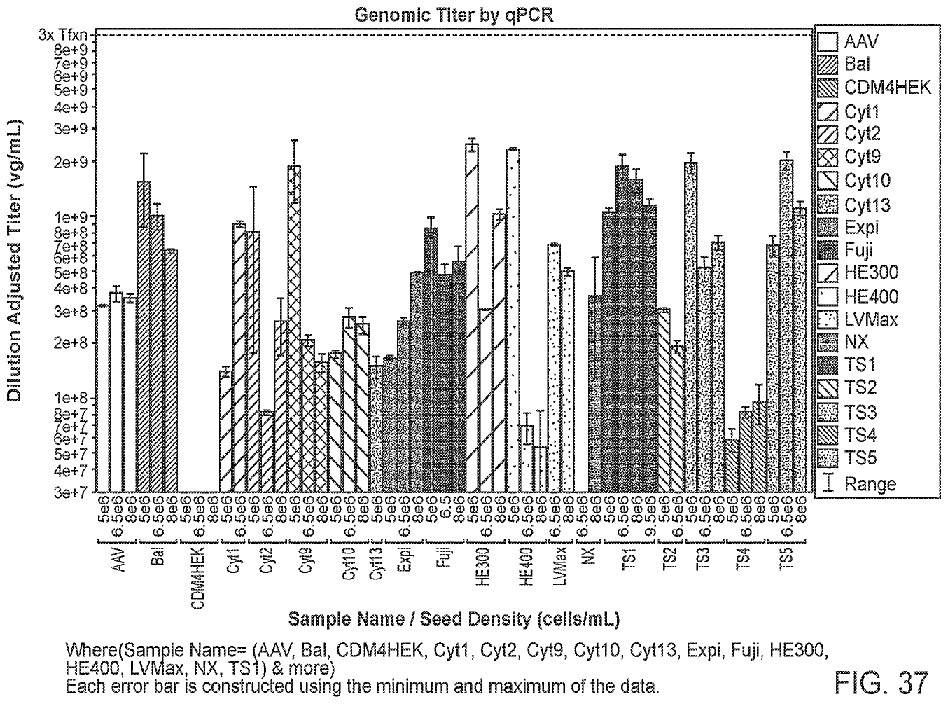

This example describes induction of the inducible stable cell lines of the present disclosure in different cell media. Cells from the T42 pool cell line (T42) were tested in 18 different cell medias. Cells were seeded at 5×10⁶ cells/mL, 6.5×10⁶ cells/mL, 8×10⁶ cells/mL, or 9.5×10⁶ cells/mL. The cells were then induced with Tamoxifen and Dox. Cells were harvested at 70% viability. Harvested cells underwent nuclease treatment (benzonase) and qPCR was performed to determine the titer of capsids encapsidating the viral genome (e.g., the payload construct). The titer of capsids encapsidating a viral genome (e.g., the payload construct) in each cell media at each seed density after induction is shown in FIG. 37.

### 6.18.12. Example 12—Induction of Inducible Stable Cell Lines

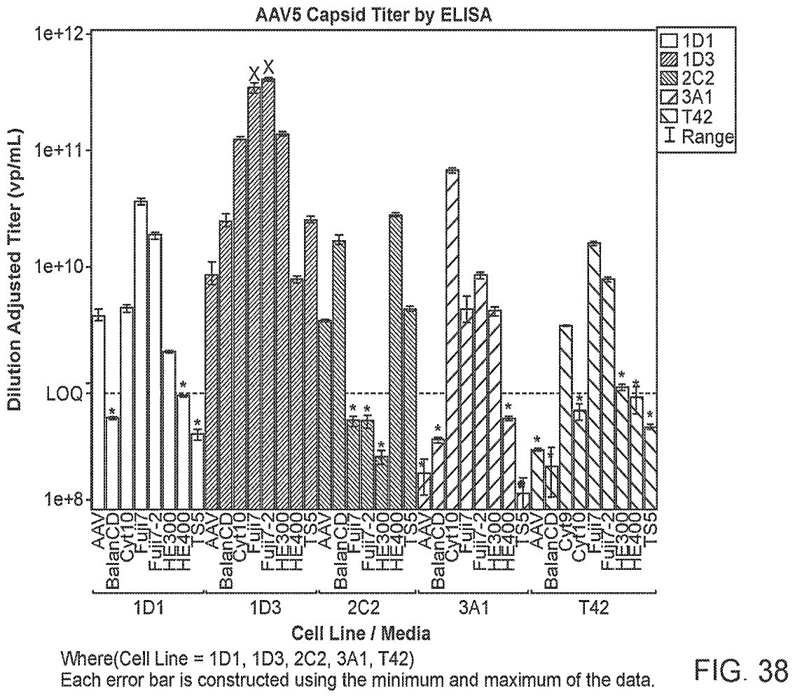

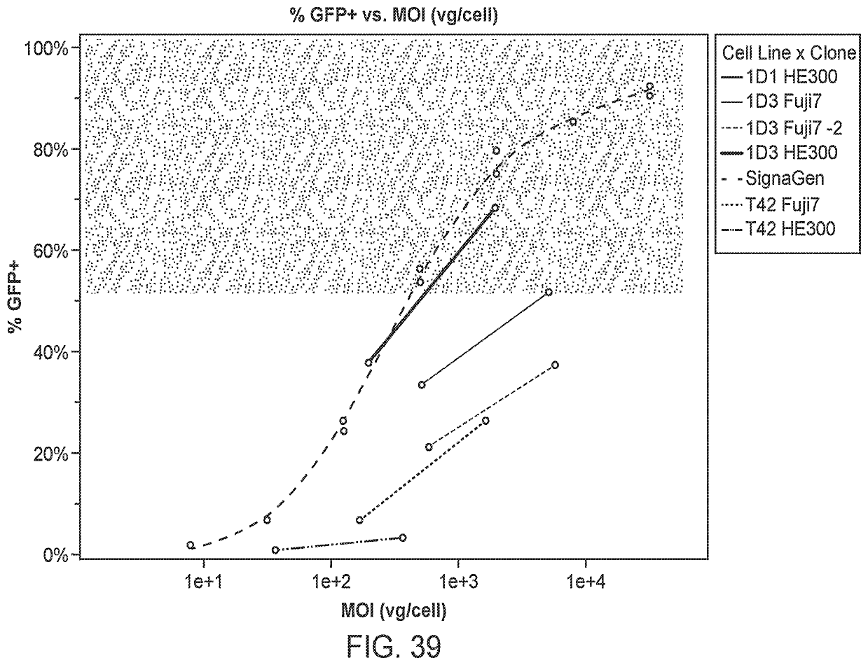

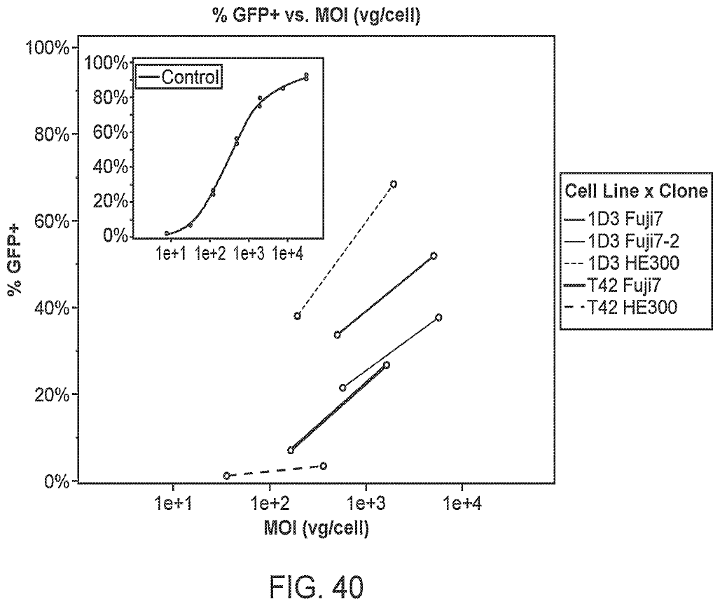

This example describes induction of mini pool clones from the T42 pool cell line compared to the T42 pool cell line. Mini pool clones from the T42 pool cell lines were passaged for a minimum of three passages. Each mini pool clone was tested in eight different media. The mini pool clones or the T42 pool cell lines cells were induced at 5×10⁶ cells/mL and then harvested 96 hours post induction with tamoxifen and doxycycline. Capsid ELISA was performed to determine total capsid titer for each mini pool clone and for the T42 pool stable cell line after induction for each of the cell medias as shown in FIG. 38. Infectivity of the capsids from select mini pool clones and for the T42 stable cell line after induction in various cell media was then tested. The percentage of GFP+ cells after infecting target cells (CHO Pro-5 cells) with capsids (payload was GFP) compared to the multiplicity of infection (MOI; vg/target cell) is shown in FIG. 39 and FIG. 40. Mini pool clone 1D3 in HE300 and and 1D3 in Fuji7 showed greater than 50% infectivity at MOIs of less than 1×10⁵.

### 6.18.13. Example 13—Bioreactor Production

This example describes production of rAAV virions in a 50 L bioreactor from stable cells as disclosed herein versus from transiently transfected cells. Triple transient transfected cells, a current stable cell line (for example, a stable cell line expanded from a clone of the T42 stable cell line of Example 12), or a new stable cell line are cultured and are induced in a 50 L bioreactor. rAAV virion production from these 50 L bioreactors is shown Table 2. Lower bioreactor titer is produced from the triple transient transfected cells compared to from a current stable cell line or from a new stable cell line. Using standard purification processes, a 40% downstream yield is produced for the rAAV virion that is produced from the triple transient transfected cells and from a current stable cell line. An increase in yield to 60% is produced when higher quality rAAV virion (e.g., higher full capsid:empty capsid ratio) is produced from a new cell line and when the purification processes is improved for a new cell line. Therefore, a higher net process yield for rAAV virion is produced from a new clone from a new stable cell line and from a clone from a current stable cell line compared to from the triple transient transfected cells.

TABLE 2
rAAV virion is produced from 50 L Bioreactor

| Basis of Design | Bioreactor | Bioreactor | Downstream | Net Process |
|---|---|---|---|---|
| Process | Scale (L) | Titer (vg/L) | Yield (%) | Yield (vg) |
| Triple | 50 | 1.00E+14 | 40% | 2.00E+15 |
| Transient | | | | |
| Transfection | | | | |
| (Current | | | | |
| Capability) | | | | |
| Current | 50 | 4.00E+14 | 40% | 8.00E+15 |
| Stable Cell Line | | | | |
| New | 50 | 1.00E+15 | 60% | 3.00E+16 |
| Stable Cell Line | | | | |

### 6.18.14. Example 14—rAAV Virion Dosing

This example describes dosing of rAAV virion for non-intravenous or intravenous administration to a patient. Table 3 shows multiplicity of infection (MOI), average dose, and yield for rAAV virion that is produced by triple transient transfect cells, current stable cell line (for example, a stable cell line expanded from a clone of the T42 stable cell line of Example 12), or a new stable cell line for either non-intravenous or intravenous administration to a patient. MOI is increased for the rAAV virion that is produced by a new stable cell line compared to by the current cell line or the triple transient transfected cells. Therefore, average dose is decreased for the rAAV virion that is produced by a new stable cell line compared to by the current cell line or the triple transient transfected cells.

TABLE 3

| Triple Transient | | | | | | | | |
|---|---|---|---|---|---|---|---|---|
| Transfection | | | Current Stable Cell Line | | | New Stable Cell Line | | |
| | Non IV | IV | | Non IV | IV | | Non IV | IV |
| MOI | 1:1000 | 1:1000 | MOI | 1:1000 | 1:1000 | MOI | 1:200 | 1:200 |
| Avg | 1.00E+12 | 4.00E+15 | Avg | 1.00E+12 | 4.00E+15 | Avg | 2.00E+11 | 8.00E+14 |
| Dose (vg) | | | Dose (vg) | | | Dose (vg) | | |
| Yield | 4.00E+13 | 4.00E+13 | Yield | 1.60E+14 | 1.60E+14 | Yield | 6.00E+14 | 6.00E+14 |
| (vg/L) | | | (vg/L) | | | | | |

Table 4 shows the number of patient doses per bath of rAAV virion that is produced by triple transient transfect cells, a current stable cell line, or a new stable cell line in a 50 L bioreactor or a 500 L bioreactor for either non-intravenous or intravenous administration to a patient. Yield is increased for the rAAV virion that is produced by a current stable cell line or a new stable cell line compared to by the triple transient transfected cells (see Table 3). Therefore, the number of doses per batch is increased for the rAAV virion that is produced by a new stable cell line or the current cell line compared to by the triple transient transfected cells.

TABLE 4

| Triple Transient Transfection | | | Current Stable Cell Line | | | New Stable Cell Line | | |
|---|---|---|---|---|---|---|---|---|
| | Non IV | IV | | Non IV | IV | | Non IV | IV |
| Scale | #Dose/Batch | #Dose/Batch | Scale | #Dose/Batch | #Dose/Batch | Scale | #Dose/Batch | #Dose/Batch |
| 50 | 2000 | 0.5 | 50 | 8000 | 2 | 50 | 150000 | 37.5 |
| 500 | 20000 | 5 | 500 | 80000 | 20 | 500 | 1500000 | 375 |

### 7. INFORMAL SEQUENCE LISTING

First spacer segment-SEQ ID NO: 1:
GTGAGTTTGGGGACCCTTGATTGTTCTTTCTTTTTCGCTATTGTAAAATTCATGTTATATGGAGGG
GGCAAAGTTTTCAGGGTGTTGTTTAGAATGGGAAGATGTCCCTTGTATCACCATGGACCCTCATGA
TAATTTTGTTTCTTTCACTTTCTACTCTGTTGACAACCATTGTCTCCTCTTATTTTCTTTTCATTT
TCTGTAACTTTTTCGTTAAACTTTAGCTTGCATTTGTAACGAATTTTTAAATTCACTTTTGTTTAT
TTGTCAGATTGTAAGTACTTTCTCTAATCACTTTTTTTTCAAGGCAATCAGGGTATATTATATTGT
ACTTCAGCACAGTTTTAGAGAAC
Second spacer segment-SEQ ID NO: 2:
ataacttcgtataatgtatgctatacgaagttatCGGGCCCCTCTGCTAACCATGTTCATGCCTTC
TTCTTTTTCCTACAGatggtgagcaagggcgaggagctgttcaccggggtggtgcccatcctggtc
gagctggacggcgacgtaaacggccacaagttcagcgtgtccggcgagggcgagggcgatgccacc
tacggcaagctgaccctgaagttcatctgcaccaccggcaagctgcccgtgccctggcccaccctc
gtgaccaccctgacctacggcgtgcagtgcttcagccgctaccccgaccacatgaagcagcacgac
ttcttcaagtccgccatgcccgaaggctacgtccaggagcgcaccatcttcttcaaggacgacggc
aactacaagacccgcgccgaggtgaagttcgagggcgacaccctggtgaaccgcatcgagctgaag
ggcatcgacttcaaggaggacggcaacatcctggggcacaagctggagtacaactacaacagccac
aacgtctatatcatggccgacaagcagaagaacggcatcaaggtgaacttcaagatccgccacaac
atcgaggacggcagcgtgcagctcgccgaccactaccagcagaacacccccatcggcgacggcccc
gtgctgctgcccgacaaccactacctgagcacccagtccgccctgagcaaagaccccaacgagaag
cgcgatcacatggtcctgctggagttcgtgaccgccgccgggatcactctcggcatggacgagctg
tacaagtaaCCTCAGGTGCAGGCTGCCTATCAGAAGGTGGTGGCTGGTGTGGCCAATGCCCTGGCT
CACAAATACCACTGAGATCTTTTTCCCTCTGCCAAAAATTATGGGGACATCATGAAGCCCCTTGAG
CATCTGACTTCTGGCTAATAAAGGAAATTTATTTTCATTGCAATAGTGTGTTGGAATTTTTTGTGT
CTCTCACTCGGAAGGACATATGGGAGGGCAAATCATTTAAAACATCAGAATGAGTATTTGGTTTAG
AGTTTGGCAACATATGCCCATATGCTGGCTGCCATGAACAAAGGTTGGCTATAAAGAGGTCATCAG
TATATGAAACAGCCCCCTGCTGTCCATTCCTTATTCCATAGAAAAGCCTTGACTTGAGGTTAGATT
TTTTTTATATTTTGTTTTGTGTTATTTTTTTCTTTAACATCCCTAAAATTTTCCTTACATGTTTTA
CTAGCCAGATTTTTCCTCCTCTCCTGACTACTCCCAGTCATAGCTGTCCCTCTTCTCTTATGGAGA
TCataacttcgtataatgtatgctatacgaagttat
Third spacer segment-SEQ ID NO: 3:
AATTGTTATAATTAAATGATAAGGTAGAATATTTCTGCATATAAATTCTGGCTGGCGTGGAAATAT
TCTTATTGGTAGAAACAACTACACCCTGGTCATCATCCTGCCTTTCTCTTTATGGTTACAATGATA
TACACTGTTTGAGATGAGGATAAAATACTCTGAGTCCAAACCGGGCCCCTCTGCTAACCATGTTCA
TGCCTTCTTCTtTTTCCTACAG
DHFR Z-Nter-SEQ ID NO: 4:
ATGAGAGGGTCAGATCCGGCAGCGTTGAAGCGGGCTAGGAACACTGAGGCAGCAAGGCGCTCTCGA
GCAAGGAAGTTGCAACGGATGAAACAATTGGAAGACAAAGTTGAAGAGCTGCTTTCAAAAAACTAC
CACCTTGAAAATGAAGTCGCGAGGCTGAAGAAATTGGTCGGATCTGCTGGCAGCGCAGCGGGGAGC
GGTGAGTTTATGGTCAGACCTCTCAACTGTATTGTCGCTGTCTCACAGAACATGGGTATCGGAAAG
AACGGTGACTTGCCGTGGCCGCCACTGCGGAATGAGTTCAAATACTTTCAGCGCATGACGACCACC
AGCAGTGTGGAGGGTAAGCAAAATCTTGTCATAATGGGTCGCAAGACTTGGTTTTCTATTCCAGAG
AAAAACAGACCGCTTAAAGATAGGATTAACATCGTGTTGAGCCGGGAACTGAAAGAGCCACCAAGG
GGAGCACATTTTTTGGCTAAGTCCTTGGATGACGCCCTGCGACTGATAGAGCAACCAGAACTTGCT
TAGTAA
DHFR Z-Cter-SEQ ID NO: 5:
ATGCGCGGTTCCGACCCAGCAGCTTTGAAACGAGCACGAAACACGGAAGCAGCCCGCAGGAGTCGA
GCGAGAAAACTTCAGCGGATGAAGCAGCTTGAAGATAAAGTCGAGGAATTGCTTAGCAAGAATTAT
CACCTCGAGAATGAAGTGGCGCGACTGAAAAAACTTGTAGGTTCTGCTGGGAGCGCAGCCGGAAGC
GGCGAGTTCTCAAAAGTTGACATGGTGTGGATCGTGGGTGGAAGTTCTGTCTATCAAGAGGCGATG
AATCAGCCTGGCCACCTCAGACTGTTTGTTACAAGGATCATGCAGGAGTTCGAGTCTGACACGTTT
TTTCCAGAGATCGACCTGGGGAAATATAAACTCCTCCCAGAGTACCCAGGAGTGCTTAGTGAGGTC
CAAGAAGAGAAGGGAATCAAATATAAATTTGAAGTTTACGAAAAGAAGGATTAGTAA
ITR deleted AAV2 genome with Construct 1 (cloned in pCRII Topo vector)
SEQ ID NO: 6
ggaggggtggagtcgtgacgtgaattacgtcatagggttagggaggtcctgtattagaggtcacgt
gagtgttttgcgacattttgcgacaccatgtggtcacgctgggtatttaagcccgagtgagcacgc
agggtctccATTTTGAAGCGGGAGGTTTGAACGCGCAGCCGCCatgccggggttttacgagattgt
gattaaggtccccagcgaccttgacgagcatctgcccggcatttctgacagctttgtgaactgggt
ggccgagaaggaatgggagttgccgccagattctgacatggatctgaatctgattgagcaggcacc
cctgaccgtggccgagaagctgcagcgcgactttctgacggaatggcgccgtgtgagtaaggcccc
ggaggcccttttctttgtgcaatttgagaagggagagagctacttccacatgcacgtgctcgtgga
aaccaccggggtgaaatccatggttttgggacgtttcctgagtcagattcgcgaaaaactgattca
gagaatttaccgcgggatcgagccgactttgccaaactggttcgcggtcacaaagaccagaaatgg
cgccggaggcgggaacaaggtggtggatgagtgctacatccccaattacttgctccccaaaaccca
gcctgagctccagtgggcgtggactaatatggaacagtatttaagcgcctgtttgaatctcacgga
gcgtaaacggttggtggcgcagcatctgacgcacgtgtcgcagacgcaggagcagaacaaagagaa
tcagaatcccaattctgatgcgccggtgatcagatcaaaaacttcagccaggtacatggagctggt
cgggtggctcgtggacaaGGTGAGTTTGGGGACCCTTGATTGTTCTTTCTTTTTCGCTATTGTAAA
ATTCATGTTATATGGAGGGGGCAAAGTTTTCAGGGTGTTGTTTAGAATGGGAAGATGTCCCTTGTA
TCACCATGGACCCTCATGATAATTTTGTTTCTTTCACTTTCTACTCTGTTGACAACCATTGTCTCC
TCTTATTTTCTTTTCATTTTCTGTAACTTTTTCGTTAAACTTTAGCTTGCATTTGTAACGAATTTT
TAAATTCACTTTTGTTTATTTGTCAGATTGTAAGTACTTTCTCTAATCACTTTTTTTTCAAGGCAA
TCAGGGTATATTATATTGTACTTCAGCACAGTTTTAGAGAACataacttcgtataatgtatgctat
acgaagttatCGGGCCCCTCTGCTAACCATGTTCATGCCTTCTTCTTTTTCCTACAGatggtgagc
aagggcgaggagctgttcaccggggtggtgcccatcctggtcgagctggacggcgacgtaaacggc
cacaagttcagcgtgtccggcgagggcgagggcgatgccacctacggcaagctgaccctgaagttc
atctgcaccaccggcaagctgcccgtgccctggcccaccctcgtgaccaccctgacctacggcgtg
cagtgcttcagccgctaccccgaccacatgaagcagcacgacttcttcaagtccgccatgcccgaa
ggctacgtccaggagcgcaccatcttcttcaaggacgacggcaactacaagacccgcgccgaggtg
aagttcgagggcgacaccctggtgaaccgcatcgagctgaagggcatcgacttcaaggaggacggc
aacatcctggggcacaagctggagtacaactacaacagccacaacgtctatatcatggccgacaag
cagaagaacggcatcaaggtgaacttcaagatccgccacaacatcgaggacggcagcgtgcagctc
gccgaccactaccagcagaacacccccatcggcgacggccccgtgctgctgcccgacaaccactac
ctgagcacccagtccgccctgagcaaagaccccaacgagaagcgcgatcacatggtcctgctggag
ttcgtgaccgccgccgggatcactctcggcatggacgagctgtacaagtaaCCTCAGGTGCAGGCT
GCCTATCAGAAGGTGGTGGCTGGTGTGGCCAATGCCCTGGCTCACAAATACCACTGAGATCTTTTT
CCCTCTGCCAAAAATTATGGGGACATCATGAAGCCCCTTGAGCATCTGACTTCTGGCTAATAAAGG
AAATTTATTTTCATTGCAATAGTGTGTTGGAATTTTTTGTGTCTCTCACTCGGAAGGACATATGGG
AGGGCAAATCATTTAAAACATCAGAATGAGTATTTGGTTTAGAGTTTGGCAACATATGCCCATATG
CTGGCTGCCATGAACAAAGGTTGGCTATAAAGAGGTCATCAGTATATGAAACAGCCCCCTGCTGTC
CATTCCTTATTCCATAGAAAAGCCTTGACTTGAGGTTAGATTTTTTTTATATTTTGTTTTGTGTTA
TTTTTTTCTTTAACATCCCTAAAATTTTCCTTACATGTTTTACTAGCCAGATTTTTCCTCCTCTCC
TGACTACTCCCAGTCATAGCTGTCCCTCTTCTCTTATGGAGATCataacttcgtataatgtatgct
atacgaagttatAATTGTTATAATTAAATGATAAGGTAGAATATTTCTGCATATAAATTCTGGCTG
GCGTGGAAATATTCTTATTGGTAGAAACAACTACACCCTGGTCATCATCCTGCCTTTCTCTTTATG
GTTACAATGATATACACTGTTTGAGATGAGGATAAAATACTCTGAGTCCAAACCGGGCCCCTCTGC
TAACCATGTTCATGCCTTCTTCTtTTTCCTACAGgggattacctcggagaagcagtggatccagga
ggaccaggcctcatacatctccttcaatgcggcctccaactcgcggtcccaaatcaaggctgcctt
ggacaatgcgggaaagattatgagcctgactaaaaccgcccccgactacctggtgggccagcagcc
cgtggaggacatttccagcaatcggatttataaaattttggaactaaacgggtacgatccccaata
tgcggcttccgtctttctgggatgggccacgaaaaagttcggcaagaggaacaccatctggctgtt
tgggcctgcaactaccgggaagaccaacatcgcggaggccatagcccacactgtgcccttctacgg
gtgcgtaaactggaccaatgagaactttcccttcaacgactgtgtcgacaagatggtgatctggtg
ggaggaggggaagatgaccgccaaggtcgtggagtcggccaaagccattctcggaggaagcaaggt
gcgcgtggaccagaaatgcaagtcctcggcccagatagacccgactcccgtgatcgtcacctccaa
caccaacatgtgcgccgtgattgacgggaactcaacgaccttcgaacaccagcagccgttgcaaga
ccggatgttcaaatttgaactcacccgccgtctggatcatgactttgggaaggtcaccaagcagga
agtcaaagactttttccgggggcaaaggatcacgtggttgaggtggagcatgaattctacgtcaaa
aagggtggagccaagaaaagacccgcccccagtgacgcagatataagtgagcccaaacgggtgcgc
gagtcAGTTGCGCagccatcgacgtcagacgcggaagcttcgatcaactacgcagacagGTACCAA
AACAAATGTTCTCGTCACGTGGGCATGAATCTGATGCTGTTTCCCTGCAGACAATGCGAGAGAATG
AATCAGAATTCAAATATCTGCTTCACTCACGGACAGAAAGACTGTTTAGAGTGCTTTCCCGTGTCA
GAATCTCAACCCGTTTCTGTCGTCAAAAAGGCGTATCAGAAACTGTGCTACATTCATCATATCATG
GGAAAGGTGCCAGACGCTTGCACTGCCTGCGATCTGGTCAATGTGGATTTGGATGACTGCATCTTT
GAACAATAAATGATTTAAATCAGGTATGGCTGCCGATGGTTATCTTCCAGattggctcgaggacac
tctctctgaaggaataagacagtggtggaagctcaaacctggcccaccaccaccaaagcccgcaga
gcggcataaggacgacagcaggggtcttgtgcttcctgggtacaagtacctcggacccttcaacgg
actcgacaagggagagccggtcaacgaggcagacgccgcggccctcgagcacgacaaagcctacga
ccggcagctcgacagcggagacaacccgtacctcaagtacaaccacgccgacgcggagtttcagga
gcgccttaaagaagatacgtcttttgggggcaacctcggacgagcagtcttccaggcgaaaaagag
ggttcttgaacctctgggcctggttgaggaacctgttaagacggctccgggaaaaaagaggccggt
agagcactctcctgtggagccagactcctcctcgggaaccggaaaggcgggccagcagcctgcaag
aaaaagattgaattttggtcagactggagacgcagactcagtacctgacccccagcctctcggaca
gccaccagcagccccctctggtctgggaactaatacgatggctacaggcagtggcgcaccaatggc
agacaataacgagggcgccgacggagtgggtaattcctcgggaaattggcattgcgattccacatg
gatgggcgacagagtcatcaccaccagcacccgaacctgggccctgcccacctacaacaaccacct
ctacaaacaaatttccagccaatcaggagcctcgaacgacaatcactactttggctacagcacccc
ttgggggtattttgacttcaacagattccactgccacttttcaccacgtgactggcaaagactcat
caacaacaactggggattccgacccaagagactcaacttcaagctctttaacattcaagtcaaaga
ggtcacgcagaatgacggtacgacgacgattgccaataaccttaccagcacggttcaggtgtttac
tgactcggagtaccagctcccgtacgtcctcggctcggcgcatcaaggatgcctcccgccgttccc
agcagacgtcttcatggtgccacagtatggatacctcaccctgaacaacgggagtcaggcagtagg
acgctcttcattttactgcctggagtactttccttctcagatgctgcgtaccggaaacaactttac
cttcagctacacttttgaggacgttcctttccacagcagctacgctcacagccagagtctggaccg
tctcatgaatcctctcatcgaccagtacctgtattacttgagcagaacaaacactccaagtggaac
caccacgcagtcaaggcttcagttttctcaggccggagcgagtgacattcgggaccagtctaggaa
ctggcttcctggaccctgttaccgccagcagcgagtatcaaagacatctgcggataacaacaacag
tgaatactcgtggactggagctaccaagtaccacctcaatggcagagactctctggtgaatccggg
cccggccatggcaagccacaaggacgatgaagaaaagttttttcctcagagcggggttctcatctt
tgggaagcaaggctcagagaaaacaaatgtggacattgaaaaggtcatgattacagacgaagagga
aatcaggacaaccaatcccgtggctacggagcagtatggttctgtatctaccaacctccagagagg
caacagacaagcagctaccgcagatgtcaacacacaaggcgttcttccaggcatggtctggcagga
cagagatgtgtaccttcaggggcccatctgggcaaagattccacacacggacggacattttcaccc
ctctcccctcatgggtggattcggacttaaacaccctcctccacagattctcatcaagaacacccc
ggtacctgcgaatccttcgaccaccttcagtgcggcaaagtttgcttccttcatcacacagtactc
cacgggacaggtcagcgtggagatcgagtgggagctgcagaaggaaaacagcaaacgctggaatcc
cgaaattcagtacacttccaactacaacaagtctgttaatgtggactttactgtggacactaatgg
cgtgtattcagagcctcgccccattggcaccagatacctgactcgtaatctgtaattgcttgttaa
tcaataaaccgtttaattcgtttcagttgaactttggtctctgcgtatttctttcttatctagttt
ccatggctacgtagataagtagcatggcgggttaatcattaactacaTAAGGGCGAATTCTGCAGA
TATCCATCACACTGGCGGCCGCTCGAGCATGCATCTAGAGGGCCCAATTCGCCCTATAGTGAGTCG
TATTACAATTCACTGGCCGTCGTTTTACAACGTCGTGACTGGGAAAACCCTGGCGTTACCCAACTT
AATCGCCTTGCAGCACATCCCCCTTTCGCCAGCTGGCGTAATAGCGAAGAGGCCCGCACCGATCGC
CCTTCCCAACAGTTGCGCAGCCTATACGTACGGCAGTTTAAGGTTTACACCTATAAAAGAGAGAGC
CGTTATCGTCTGTTTGTGGATGTACAGAGTGATATTATTGACACGCCGGGGCGACGGATGGTGATC
CCCCTGGCCAGTGCACGTCTGCTGTCAGATAAAGTCTCCCGTGAACTTTACCCGGTGGTGCATATC
GGGGATGAAAGCTGGCGCATGATGACCACCGATATGGCCAGTGTGCCGGTCTCCGTTATCGGGGAA
GAAGTGGCTGATCTCAGCCACCGCGAAAATGACATCAAAAACGCCATTAACCTGATGTTCTGGGGA
ATATAAATGTCAGGCATGAGATTATCAAAAAGGATCTTCACCTAGATCCTTTTCACGTAGAAAGCC
AGTCCGCAGAAACGGTGCTGACCCCGGATGAATGTCAGCTACTGGGCTATCTGGACAAGGGAAAAC
GCAAGCGCAAAGAGAAAGCAGGTAGCTTGCAGTGGGCTTACATGGCGATAGCTAGACTGGGCGGTT
TTATGGACAGCAAGCGAACCGGAATTGCCAGCTGGGGCGCCCTCTGGTAAGGTTGGGAAGCCCTGC
AAAGTAAACTGGATGGCTTTCTCGCCGCCAAGGATCTGATGGCGCAGGGGATCAAGCTCTGATCAA
GAGACAGGATGAGGATCGTTTCGCATGATTGAACAAGATGGATTGCACGCAGGTTCTCCGGCCGCT
TGGGTGGAGAGGCTATTCGGCTATGACTGGGCACAACAGACAATCGGCTGCTCTGATGCCGCCGTG
TTCCGGCTGTCAGCGCAGGGGCGCCCGGTTCTTTTTGTCAAGACCGACCTGTCCGGTGCCCTGAAT
GAACTGCAAGACGAGGCAGCGCGGCTATCGTGGCTGGCCACGACGGGCGTTCCTTGCGCAGCTGTG
CTCGACGTTGTCACTGAAGCGGGAAGGGACTGGCTGCTATTGGGCGAAGTGCCGGGGCAGGATCTC
CTGTCATCTCACCTTGCTCCTGCCGAGAAAGTATCCATCATGGCTGATGCAATGCGGCGGCTGCAT
ACGCTTGATCCGGCTACCTGCCCATTCGACCACCAAGCGAAACATCGCATCGAGCGAGCACGTACT
CGGATGGAAGCCGGTCTTGTCGATCAGGATGATCTGGACGAAGAGCATCAGGGGCTCGCGCCAGCC
GAACTGTTCGCCAGGCTCAAGGCGAGCATGCCCGACGGCGAGGATCTCGTCGTGACCCATGGCGAT
GCCTGCTTGCCGAATATCATGGTGGAAAATGGCCGCTTTTCTGGATTCATCGACTGTGGCCGGCTG
GGTGTGGCGGACCGCTATCAGGACATAGCGTTGGCTACCCGTGATATTGCTGAAGAGCTTGGCGGC
GAATGGGCTGACCGCTTCCTCGTGCTTTACGGTATCGCCGCTCCCGATTCGCAGCGCATCGCCTTC
TATCGCCTTCTTGACGAGTTCTTCTGAATTATTAACGCTTACAATTTCCTGATGCGGTATTTTCTC
CTTACGCATCTGTGCGGTATTTCACACCGCATACAGGTGGCACTTTTCGGGGAAATGTGCGCGGAA
CCCCTATTTGTTTATTTTTCTAAATACATTCAAATATGTATCCGCTCATGAGACAATAACCCTGAT
AAATGCTTCAATAATAGCACGTGAGGAGGGCCACCATGGCCAAGTTGACCAGTGCCGTTCCGGTGC
TCACCGCGCGCGACGTCGCCGGAGCGGTCGAGTTCTGGACCGACCGGCTCGGGTTCTCCCGGGACT
TCGTGGAGGACGACTTCGCCGGTGTGGTCCGGGACGACGTGACCCTGTTCATCAGCGCGGTCCAGG
ACCAGGTGGTGCCGGACAACACCCTGGCCTGGGTGTGGGTGCGCGGCCTGGACGAGCTGTACGCCG
AGTGGTCGGAGGTCGTGTCCACGAACTTCCGGGACGCCTCCGGGCCGGCCATGACCGAGATCGGCG
AGCAGCCGTGGGGGCGGGAGTTCGCCCTGCGCGACCCGGCCGGCAACTGCGTGCACTTCGTGGCCG
AGGAGCAGGACTGACACGTGCTAAAACTTCATTTTTAATTTAAAAGGATCTAGGTGAAGATCCTTT
TTGATAATCTCATGACCAAAATCCCTTAACGTGAGTTTTCGTTCCACTGAGCGTCAGACCCCGTAG
AAAAGATCAAAGGATCTTCTTGAGATCCTTTTTTTCTGCGCGTAATCTGCTGCTTGCAAACAAAAA
AACCACCGCTACCAGCGGTGGTTTGTTTGCCGGATCAAGAGCTACCAACTCTTTTTCCGAAGGTAA
CTGGCTTCAGCAGAGCGCAGATACCAAATACTGTCCTTCTAGTGTAGCCGTAGTTAGGCCACCACT
TCAAGAACTCTGTAGCACCGCCTACATACCTCGCTCTGCTAATCCTGTTACCAGTGGCTGCTGCCA
GTGGCGATAAGTCGTGTCTTACCGGGTTGGACTCAAGACGATAGTTACCGGATAAGGCGCAGCGGT
CGGGCTGAACGGGGGGTTCGTGCACACAGCCCAGCTTGGAGCGAACGACCTACACCGAACTGAGAT
ACCTACAGCGTGAGCTATGAGAAAGCGCCACGCTTCCCGAAGGGAGAAAGGCGGACAGGTATCCGG
TAAGCGGCAGGGTCGGAACAGGAGAGCGCACGAGGGAGCTTCCAGGGGGAAACGCCTGGTATCTTT
ATAGTCCTGTCGGGTTTCGCCACCTCTGACTTGAGCGTCGATTTTTGTGATGCTCGTCAGGGGGGC
GGAGCCTATGGAAAAACGCCAGCAACGCGGCCTTTTTACGGTTCCTGGGCTTTTGCTGGCCTTTTG
CTCACATGTTCTTTCCTGCGTTATCCCCTGATTCTGTGGATAACCGTATTACCGCCTTTGAGTGAG
CTGATACCGCTCGCCGCAGCCGAACGACCGAGCGCAGCGAGTCAGTGAGCGAGGAAGCGGAAGAGC
GCCCAATACGCAAACCGCCTCTCCCCGCGCGTTGGCCGATTCATTAATGCAGCTGGCACGACAGGT
TTCCCGACTGGAAAGCGGGCAGTGAGCGCAACGCAATTAATGTGAGTTAGCTCACTCATTAGGCAC
CCCAGGCTTTACACTTTATGCTTCCGGCTCGTATGTTGTGTGGAATTGTGAGCGGATAACAATTTC
ACACAGGAAACAGCTATGACCATGATTACGCCAAGCTATTTAGGTGACACTATAGAATACTCAAGC
TATGCATCAAGCTTGGTACCGAGCTCGGATCCACTAGTAACGGCCGCCAGTGTGCTGGAATTCGCC
CT
SEQ ID NO: 7 (Rep/Cap construct for AAV2)
GGCCTCCACGGCCACTAGTAACGGCCGCCAGTGTGCTGGAATTCGCCCTGGAGGGGTGGAGTCGTG
ACGTGAATTACGTCATAGGGTTAGGGAGGTCCTGTATTAGAGGTCACGTGAGTGTTTTGCGACATT
TTGCGACACCATGTGGTCACGCTGGGTATTTAAGCCCGAGTGAGCACGCAGGGTCTCCATTTTGAA
GCGGGAGGTTTGAACGCGCAGCCGCCATGCCGGGGTTTTACGAGATTGTGATTAAGGTCCCCAGCG
ACCTTGACGAGCATCTGCCCGGCATTTCTGACAGCTTTGTGAACTGGGTGGCCGAGAAGGAATGGG
AGTTGCCGCCAGATTCTGACATGGATCTGAATCTGATTGAGCAGGCACCCCTGACCGTGGCCGAGA
AGCTGCAGCGCGACTTTCTGACGGAATGGCGCCGTGTGAGTAAGGCCCCGGAGGCCCTTTTCTTTG
TGCAATTTGAGAAGGGAGAGAGCTACTTCCACATGCACGTGCTCGTGGAAACCACCGGGGTGAAAT
CCATGGTTTTGGGACGTTTCCTGAGTCAGATTCGCGAAAAACTGATTCAGAGAATTTACCGCGGGA
TCGAGCCGACTTTGCCAAACTGGTTCGCGGTCACAAAGACCAGAAATGGCGCCGGAGGCGGGAACA
AGGTGGTGGATGAGTGCTACATCCCCAATTACTTGCTCCCCAAAACCCAGCCTGAGCTCCAGTGGG
CGTGGACTAATATGGAACAGTATTTAAGCGCCTGTTTGAATCTCACGGAGCGTAAACGGTTGGTGG
CGCAGCATCTGACGCACGTGTCGCAGACGCAGGAGCAGAACAAAGAGAATCAGAATCCCAATTCTG
ATGCGCCGGTGATCAGATCAAAAACTTCAGCCAGGTACATGGAGCTGGTCGGGTGGCTCGTGGACA
AGGTGAGTTTGGGGACCCTTGATTGTTCTTTCTTTTTCGCTATTGTAAAATTCATGTTATATGGAG
GGGGCAAAGTTTTCAGGGTGTTGTTTAGAATGGGAAGATGTCCCTTGTATCACCATGGACCCTCAT
GATAATTTTGTTTCTTTCACTTTCTACTCTGTTGACAACCATTGTCTCCTCTTATTTTCTTTTCAT
TTTCTGTAACTTTTTCGTTAAACTTTAGCTTGCATTTGTAACGAATTTTTAAATTCACTTTTGTTT
ATTTGTCAGATTGTAAGTACTTTCTCTAATCACTTTTTTTTCAAGGCAATCAGGGTATATTATATT
GTACTTCAGCACAGTTTTAGAGAACATAACTTCGTATAATGTATGCTATACGAAGTTATCGGGCCC
CTCTGCTAACCATGTTCATGCCTTCTTCTTTTTCCTACAGATGGTGAGCAAGGGCGAGGAGCTGTT
CACCGGGGTGGTGCCCATCCTGGTCGAGCTGGACGGCGACGTAAACGGCCACAAGTTCAGCGTGTC
CGGCGAGGGCGAGGGCGATGCCACCTACGGCAAGCTGACCCTGAAGTTCATCTGCACCACCGGCAA
GCTGCCCGTGCCCTGGCCCACCCTCGTGACCACCCTGACCTACGGCGTGCAGTGCTTCAGCCGCTA
CCCCGACCACATGAAGCAGCACGACTTCTTCAAGTCCGCCATGCCCGAAGGCTACGTCCAGGAGCG
CACCATCTTCTTCAAGGACGACGGCAACTACAAGACCCGCGCCGAGGTGAAGTTCGAGGGCGACAC
CCTGGTGAACCGCATCGAGCTGAAGGGCATCGACTTCAAGGAGGACGGCAACATCCTGGGGCACAA
GCTGGAGTACAACTACAACAGCCACAACGTCTATATCATGGCCGACAAGCAGAAGAACGGCATCAA
GGTGAACTTCAAGATCCGCCACAACATCGAGGACGGCAGCGTGCAGCTCGCCGACCACTACCAGCA
GAACACCCCCATCGGCGACGGCCCCGTGCTGCTGCCCGACAACCACTACCTGAGCACCCAGTCCGC
CCTGAGCAAAGACCCCAACGAGAAGCGCGATCACATGGTCCTGCTGGAGTTCGTGACCGCCGCCGG
GATCACTCTCGGCATGGACGAGCTGTACAAGTAACCTCAGGTGCAGGCTGCCTATCAGAAGGTGGT
GGCTGGTGTGGCCAATGCCCTGGCTCACAAATACCACTGAGATCTTTTTCCCTCTGCCAAAAATTA
TGGGGACATCATGAAGCCCCTTGAGCATCTGACTTCTGGCTAATAAAGGAAATTTATTTTCATTGC
AATAGTGTGTTGGAATTTTTTGTGTCTCTCACTCGGAAGGACATATGGGAGGGCAAATCATTTAAA
ACATCAGAATGAGTATTTGGTTTAGAGTTTGGCAACATATGCCCATATGCTGGCTGCCATGAACAA
AGGTTGGCTATAAAGAGGTCATCAGTATATGAAACAGCCCCCTGCTGTCCATTCCTTATTCCATAG
AAAAGCCTTGACTTGAGGTTAGATTTTTTTTATATTTTGTTTTGTGTTATTTTTTTCTTTAACATC
CCTAAAATTTTCCTTACATGTTTTACTAGCCAGATTTTTCCTCCTCTCCTGACTACTCCCAGTCAT
AGCTGTCCCTCTTCTCTTATGGAGATCATAACTTCGTATAATGTATGCTATACGAAGTTATAATTG
TTATAATTAAATGATAAGGTAGAATATTTCTGCATATAAATTCTGGCTGGCGTGGAAATATTCTTA
TTGGTAGAAACAACTACACCCTGGTCATCATCCTGCCTTTCTCTTTATGGTTACAATGATATACAC
TGTTTGAGATGAGGATAAAATACTCTGAGTCCAAACCGGGCCCCTCTGCTAACCATGTTCATGCCT
TCTTCTTTTTCCTACAGGGGATTACCTCGGAGAAGCAGTGGATCCAGGAGGACCAGGCCTCATACA
TCTCCTTCAATGCGGCCTCCAACTCGCGGTCCCAAATCAAGGCTGCCTTGGACAATGCGGGAAAGA
TTATGAGCCTGACTAAAACCGCCCCCGACTACCTGGTGGGCCAGCAGCCCGTGGAGGACATTTCCA
GCAATCGGATTTATAAAATTTTGGAACTAAACGGGTACGATCCCCAATATGCGGCTTCCGTCTTTC
TGGGATGGGCCACGAAAAAGTTCGGCAAGAGGAACACCATCTGGCTGTTTGGGCCTGCAACTACCG
GGAAGACCAACATCGCGGAGGCCATAGCCCACACTGTGCCCTTCTACGGGTGCGTAAACTGGACCA
ATGAGAACTTTCCCTTCAACGACTGTGTCGACAAGATGGTGATCTGGTGGGAGGAGGGGAAGATGA
CCGCCAAGGTCGTGGAGTCGGCCAAAGCCATTCTCGGAGGAAGCAAGGTGCGCGTGGACCAGAAAT
GCAAGTCCTCGGCCCAGATAGACCCGACTCCCGTGATCGTCACCTCCAACACCAACATGTGCGCCG
TGATTGACGGGAACTCAACGACCTTCGAACACCAGCAGCCGTTGCAAGACCGGATGTTCAAATTTG
AACTCACCCGCCGTCTGGATCATGACTTTGGGAAGGTCACCAAGCAGGAAGTCAAAGACTTTTTCC
GGTGGGCAAAGGATCACGTGGTTGAGGTGGAGCATGAATTCTACGTCAAAAAGGGTGGAGCCAAGA
AAAGACCCGCCCCCAGTGACGCAGATATAAGTGAGCCCAAACGGGTGCGCGAGTCAGTTGCGCAGC
CATCGACGTCAGACGCGGAAGCTTCGATCAACTACGCAGACAGGTACCAAAACAAATGTTCTCGTC
ACGTGGGCATGAATCTGATGCTGTTTCCCTGCAGACAATGCGAGAGAATGAATCAGAATTCAAATA
TCTGCTTCACTCACGGACAGAAAGACTGTTTAGAGTGCTTTCCCGTGTCAGAATCTCAACCCGTTT
CTGTCGTCAAAAAGGCGTATCAGAAACTGTGCTACATTCATCATATCATGGGAAAGGTGCCAGACG
CTTGCACTGCCTGCGATCTGGTCAATGTGGATTTGGATGACTGCATCTTTGAACAATAAATGATTT
AAATCAGGTATGGCTGCCGATGGTTATCTTCCAGATTGGCTCGAGGACACTCTCTCTGAAGGAATA
AGACAGTGGTGGAAGCTCAAACCTGGCCCACCACCACCAAAGCCCGCAGAGCGGCATAAGGACGAC
AGCAGGGGTCTTGTGCTTCCTGGGTACAAGTACCTCGGACCCTTCAACGGACTCGACAAGGGAGAG
CCGGTCAACGAGGCAGACGCCGCGGCCCTCGAGCACGACAAAGCCTACGACCGGCAGCTCGACAGC
GGAGACAACCCGTACCTCAAGTACAACCACGCCGACGCGGAGTTTCAGGAGCGCCTTAAAGAAGAT
ACGTCTTTTGGGGGCAACCTCGGACGAGCAGTCTTCCAGGCGAAAAAGAGGGTTCTTGAACCTCTG
GGCCTGGTTGAGGAACCTGTTAAGACGGCTCCGGGAAAAAAGAGGCCGGTAGAGCACTCTCCTGTG
GAGCCAGACTCCTCCTCGGGAACCGGAAAGGCGGGCCAGCAGCCTGCAAGAAAAAGATTGAATTTT
GGTCAGACTGGAGACGCAGACTCAGTACCTGACCCCCAGCCTCTCGGACAGCCACCAGCAGCCCCC
TCTGGTCTGGGAACTAATACGATGGCTACAGGCAGTGGCGCACCAATGGCAGACAATAACGAGGGC
GCCGACGGAGTGGGTAATTCCTCGGGAAATTGGCATTGCGATTCCACATGGATGGGCGACAGAGTC
ATCACCACCAGCACCCGAACCTGGGCCCTGCCCACCTACAACAACCACCTCTACAAACAAATTTCC
AGCCAATCAGGAGCCTCGAACGACAATCACTACTTTGGCTACAGCACCCCTTGGGGGTATTTTGAC
TTCAACAGATTCCACTGCCACTTTTCACCACGTGACTGGCAAAGACTCATCAACAACAACTGGGGA
TTCCGACCCAAGAGACTCAACTTCAAGCTCTTTAACATTCAAGTCAAAGAGGTCACGCAGAATGAC
GGTACGACGACGATTGCCAATAACCTTACCAGCACGGTTCAGGTGTTTACTGACTCGGAGTACCAG
CTCCCGTACGTCCTCGGCTCGGCGCATCAAGGATGCCTCCCGCCGTTCCCAGCAGACGTCTTCATG
GTGCCACAGTATGGATACCTCACCCTGAACAACGGGAGTCAGGCAGTAGGACGCTCTTCATTTTAC
TGCCTGGAGTACTTTCCTTCTCAGATGCTGCGTACCGGAAACAACTTTACCTTCAGCTACACTTTT
GAGGACGTTCCTTTCCACAGCAGCTACGCTCACAGCCAGAGTCTGGACCGTCTCATGAATCCTCTC
ATCGACCAGTACCTGTATTACTTGAGCAGAACAAACACTCCAAGTGGAACCACCACGCAGTCAAGG
CTTCAGTTTTCTCAGGCCGGAGCGAGTGACATTCGGGACCAGTCTAGGAACTGGCTTCCTGGACCC
TGTTACCGCCAGCAGCGAGTATCAAAGACATCTGCGGATAACAACAACAGTGAATACTCGTGGACT
GGAGCTACCAAGTACCACCTCAATGGCAGAGACTCTCTGGTGAATCCGGGCCCGGCCATGGCAAGC
CACAAGGACGATGAAGAAAAGTTTTTTCCTCAGAGCGGGGTTCTCATCTTTGGGAAGCAAGGCTCA
GAGAAAACAAATGTGGACATTGAAAAGGTCATGATTACAGACGAAGAGGAAATCAGGACAACCAAT
CCCGTGGCTACGGAGCAGTATGGTTCTGTATCTACCAACCTCCAGAGAGGCAACAGACAAGCAGCT
ACCGCAGATGTCAACACACAAGGCGTTCTTCCAGGCATGGTCTGGCAGGACAGAGATGTGTACCTT
CAGGGGCCCATCTGGGCAAAGATTCCACACACGGACGGACATTTTCACCCCTCTCCCCTCATGGGT
GGATTCGGACTTAAACACCCTCCTCCACAGATTCTCATCAAGAACACCCCGGTACCTGCGAATCCT
TCGACCACCTTCAGTGCGGCAAAGTTTGCTTCCTTCATCACACAGTACTCCACGGGACAGGTCAGC
GTGGAGATCGAGTGGGAGCTGCAGAAGGAAAACAGCAAACGCTGGAATCCCGAAATTCAGTACACT
TCCAACTACAACAAGTCTGTTAATGTGGACTTTACTGTGGACACTAATGGCGTGTATTCAGAGCCT
CGCCCCATTGGCACCAGATACCTGACTCGTAATCTGTAATTGCTTGTTAATCAATAAACCGTTTAA
TTCGTTTCAGTTGAACTTTGGTCTCTGCGTATTTCTTTCTTATCTAGTTTCCATGGCTACGTAGAT
AAGTAGCATGGCGGGTTAATCATTAACTACA
SEQ ID NO: 8 (STXC0068)
ACTCTTCCTTTTTCAATATTATTGAAGCATTTATCAGGGTTATTGTCTCATGAGCGGATACATATT
TGAATGTATTTAGAAAAATAAACAAATAGGGGTTCCGCGCACATTTCCCCGAAAAGTGCCACCTAA
ATTGTAAGCGTTAATATTTTGTTAAAATTCGCGTTAAATTTTTGTTAAATCAGCTCATTTTTTAAC
CAATAGGCCGAAATCGGCAAAATCCCTTATAAATCAAAAGAATAGACCGAGATAGGGTTGAGTGTT
GTTCCAGTTTGGAACAAGAGTCCACTATTAAAGAACGTGGACTCCAACGTCAAAGGGCGAAAAACC
GTCTATCAGGGCGATGGCCCACTACGTGAACCATCACCCTAATCAAGTTTTTTGGGGTCGAGGTGC
CGTAAAGCACTAAATCGGAACCCTAAAGGGAGCCCCCGATTTAGAGCTTGACGGGGAAAGCCGGCG
AACGTGGCGAGAAAGGAAGGGAAGAAAGCGAAAGGAGCGGGCGCTAGGGCGCTGGCAAGTGTAGCG
GTCACGCTGCGCGTAACCACCACACCCGCCGCGCTTAATGCGCCGCTACAGGGCGCGTCCCATTCG
CCATTCAGGCTGCGCAACTGTTGGGAAGGGCGATCGGTGCGGGCCTCTTCGCTATTACGCCAGCTG
GCGAAAGGGGGATGTGCTGCAAGGCGATTAAGTTGGGTAACGCCAGGGTTTTCCCAGTCACGACGT
TGTAAAACGACGGCCAGTGAGCGCGCCTCGTTCATTCACGTTTTTGAACCCGTGGAGGACGGGCAG
ACTCGCGGTGCAAATGTGTTTTACAGCGTGATGGAGCAGATGAAGATGCTCGACACGCTGCAGAAC
ACGCAGCTAGATTAACCCTAGAAAGATAATCATATTGTGACGTACGTTAAAGATAATCATGTGTAA
AATTGACGCATGTGTTTTATCGGTCTGTATATCGAGGTTTATTTATTAATTTGAATAGATATTAAG
TTTTATTATATTTACACTTACATACTAATAATAAATTCAACAAACAATTTATTTATGTTTATTTAT
TTATTAAAAAAAACAAAAACTCAAAATTTCTTCTATAAAGTAACAAAACTTTTATGAGGGACAGCC
CCCCCCCAAAGCCCCCAGGGATGTAATTACGTCCCTCCCCCGCTAGGGGGCAGCAGCGAGCCGCCC
GGGGCTCCGCTCCGGTCCGGCGCTCCCCCCGCATCCCCGAGCCGGCAGCGTGCGGGGACAGCCCGG
GCACGGGGAAGGTGGCACGGGATCGCTTTCCTCTGAACGCTTCTCGCTGCTCTTTGAGCCTGCAGA
CACCTGGGGGGATACGGGGAAAAGGCCTCCACGGCCACTAGTAACGGCCGCCAGTGTGCTGGAATT
CGCCCTGGAGGGGTGGAGTCGTGACGTGAATTACGTCATAGGGTTAGGGAGGTCCTGTATTAGAGG
TCACGTGAGTGTTTTGCGACATTTTGCGACACCATGTGGTCACGCTGGGTATTTAAGCCCGAGTGA
GCACGCAGGGTCTCCATTTTGAAGCGGGAGGTTTGAACGCGCAGCCGCCATGCCGGGGTTTTACGA
GATTGTGATTAAGGTCCCCAGCGACCTTGACGAGCATCTGCCCGGCATTTCTGACAGCTTTGTGAA
CTGGGTGGCCGAGAAGGAATGGGAGTTGCCGCCAGATTCTGACATGGATCTGAATCTGATTGAGCA
GGCACCCCTGACCGTGGCCGAGAAGCTGCAGCGCGACTTTCTGACGGAATGGCGCCGTGTGAGTAA
GGCCCCGGAGGCCCTTTTCTTTGTGCAATTTGAGAAGGGAGAGAGCTACTTCCACATGCACGTGCT
CGTGGAAACCACCGGGGTGAAATCCATGGTTTTGGGACGTTTCCTGAGTCAGATTCGCGAAAAACT
GATTCAGAGAATTTACCGCGGGATCGAGCCGACTTTGCCAAACTGGTTCGCGGTCACAAAGACCAG
AAATGGCGCCGGAGGCGGGAACAAGGTGGTGGATGAGTGCTACATCCCCAATTACTTGCTCCCCAA
AACCCAGCCTGAGCTCCAGTGGGCGTGGACTAATATGGAACAGTATTTAAGCGCCTGTTTGAATCT
CACGGAGCGTAAACGGTTGGTGGCGCAGCATCTGACGCACGTGTCGCAGACGCAGGAGCAGAACAA
AGAGAATCAGAATCCCAATTCTGATGCGCCGGTGATCAGATCAAAAACTTCAGCCAGGTACATGGA
GCTGGTCGGGTGGCTCGTGGACAAGGTGAGTTTGGGGACCCTTGATTGTTCTTTCTTTTTCGCTAT
TGTAAAATTCATGTTATATGGAGGGGGCAAAGTTTTCAGGGTGTTGTTTAGAATGGGAAGATGTCC
CTTGTATCACCATGGACCCTCATGATAATTTTGTTTCTTTCACTTTCTACTCTGTTGACAACCATT
GTCTCCTCTTATTTTCTTTTCATTTTCTGTAACTTTTTCGTTAAACTTTAGCTTGCATTTGTAACG
AATTTTTAAATTCACTTTTGTTTATTTGTCAGATTGTAAGTACTTTCTCTAATCACTTTTTTTTCA
AGGCAATCAGGGTATATTATATTGTACTTCAGCACAGTTTTAGAGAACATAACTTCGTATAATGTA
TGCTATACGAAGTTATCGGGCCCCTCTGCTAACCATGTTCATGCCTTCTTCTTTTTCCTACAGATG
GTGAGCAAGGGCGAGGAGCTGTTCACCGGGGTGGTGCCCATCCTGGTCGAGCTGGACGGCGACGTA
AACGGCCACAAGTTCAGCGTGTCCGGCGAGGGCGAGGGCGATGCCACCTACGGCAAGCTGACCCTG
AAGTTCATCTGCACCACCGGCAAGCTGCCCGTGCCCTGGCCCACCCTCGTGACCACCCTGACCTAC
GGCGTGCAGTGCTTCAGCCGCTACCCCGACCACATGAAGCAGCACGACTTCTTCAAGTCCGCCATG
CCCGAAGGCTACGTCCAGGAGCGCACCATCTTCTTCAAGGACGACGGCAACTACAAGACCCGCGCC
GAGGTGAAGTTCGAGGGCGACACCCTGGTGAACCGCATCGAGCTGAAGGGCATCGACTTCAAGGAG
GACGGCAACATCCTGGGGCACAAGCTGGAGTACAACTACAACAGCCACAACGTCTATATCATGGCC
GACAAGCAGAAGAACGGCATCAAGGTGAACTTCAAGATCCGCCACAACATCGAGGACGGCAGCGTG
CAGCTCGCCGACCACTACCAGCAGAACACCCCCATCGGCGACGGCCCCGTGCTGCTGCCCGACAAC
CACTACCTGAGCACCCAGTCCGCCCTGAGCAAAGACCCCAACGAGAAGCGCGATCACATGGTCCTG
CTGGAGTTCGTGACCGCCGCCGGGATCACTCTCGGCATGGACGAGCTGTACAAGTAACCTCAGGTG
CAGGCTGCCTATCAGAAGGTGGTGGCTGGTGTGGCCAATGCCCTGGCTCACAAATACCACTGAGAT
CTTTTTCCCTCTGCCAAAAATTATGGGGACATCATGAAGCCCCTTGAGCATCTGACTTCTGGCTAA
TAAAGGAAATTTATTTTCATTGCAATAGTGTGTTGGAATTTTTTGTGTCTCTCACTCGGAAGGACA
TATGGGAGGGCAAATCATTTAAAACATCAGAATGAGTATTTGGTTTAGAGTTTGGCAACATATGCC
CATATGCTGGCTGCCATGAACAAAGGTTGGCTATAAAGAGGTCATCAGTATATGAAACAGCCCCCT
GCTGTCCATTCCTTATTCCATAGAAAAGCCTTGACTTGAGGTTAGATTTTTTTTATATTTTGTTTT
GTGTTATTTTTTTCTTTAACATCCCTAAAATTTTCCTTACATGTTTTACTAGCCAGATTTTTCCTC
CTCTCCTGACTACTCCCAGTCATAGCTGTCCCTCTTCTCTTATGGAGATCATAACTTCGTATAATG
TATGCTATACGAAGTTATAATTGTTATAATTAAATGATAAGGTAGAATATTTCTGCATATAAATTC
TGGCTGGCGTGGAAATATTCTTATTGGTAGAAACAACTACACCCTGGTCATCATCCTGCCTTTCTC
TTTATGGTTACAATGATATACACTGTTTGAGATGAGGATAAAATACTCTGAGTCCAAACCGGGCCC
CTCTGCTAACCATGTTCATGCCTTCTTCTTTTTCCTACAGGGGATTACCTCGGAGAAGCAGTGGAT
CCAGGAGGACCAGGCCTCATACATCTCCTTCAATGCGGCCTCCAACTCGCGGTCCCAAATCAAGGC
TGCCTTGGACAATGCGGGAAAGATTATGAGCCTGACTAAAACCGCCCCCGACTACCTGGTGGGCCA
GCAGCCCGTGGAGGACATTTCCAGCAATCGGATTTATAAAATTTTGGAACTAAACGGGTACGATCC
CCAATATGCGGCTTCCGTCTTTCTGGGATGGGCCACGAAAAAGTTCGGCAAGAGGAACACCATCTG
GCTGTTTGGGCCTGCAACTACCGGGAAGACCAACATCGCGGAGGCCATAGCCCACACTGTGCCCTT
CTACGGGTGCGTAAACTGGACCAATGAGAACTTTCCCTTCAACGACTGTGTCGACAAGATGGTGAT
CTGGTGGGAGGAGGGGAAGATGACCGCCAAGGTCGTGGAGTCGGCCAAAGCCATTCTCGGAGGAAG
CAAGGTGCGCGTGGACCAGAAATGCAAGTCCTCGGCCCAGATAGACCCGACTCCCGTGATCGTCAC
CTCCAACACCAACATGTGCGCCGTGATTGACGGGAACTCAACGACCTTCGAACACCAGCAGCCGTT
GCAAGACCGGATGTTCAAATTTGAACTCACCCGCCGTCTGGATCATGACTTTGGGAAGGTCACCAA
GCAGGAAGTCAAAGACTTTTTCCGGTGGGCAAAGGATCACGTGGTTGAGGTGGAGCATGAATTCTA
CGTCAAAAAGGGTGGAGCCAAGAAAAGACCCGCCCCCAGTGACGCAGATATAAGTGAGCCCAAACG
GGTGCGCGAGTCAGTTGCGCAGCCATCGACGTCAGACGCGGAAGCTTCGATCAACTACGCAGACAG
GTACCAAAACAAATGTTCTCGTCACGTGGGCATGAATCTGATGCTGTTTCCCTGCAGACAATGCGA
GAGAATGAATCAGAATTCAAATATCTGCTTCACTCACGGACAGAAAGACTGTTTAGAGTGCTTTCC
CGTGTCAGAATCTCAACCCGTTTCTGTCGTCAAAAAGGCGTATCAGAAACTGTGCTACATTCATCA
TATCATGGGAAAGGTGCCAGACGCTTGCACTGCCTGCGATCTGGTCAATGTGGATTTGGATGACTG
CATCTTTGAACAATAAATGATTTAAATCAGGTATGGCTGCCGATGGTTATCTTCCAGATTGGCTCG
AGGACACTCTCTCTGAAGGAATAAGACAGTGGTGGAAGCTCAAACCTGGCCCACCACCACCAAAGC
CCGCAGAGCGGCATAAGGACGACAGCAGGGGTCTTGTGCTTCCTGGGTACAAGTACCTCGGACCCT
TCAACGGACTCGACAAGGGAGAGCCGGTCAACGAGGCAGACGCCGCGGCCCTCGAGCACGACAAAG
CCTACGACCGGCAGCTCGACAGCGGAGACAACCCGTACCTCAAGTACAACCACGCCGACGCGGAGT
TTCAGGAGCGCCTTAAAGAAGATACGTCTTTTGGGGGCAACCTCGGACGAGCAGTCTTCCAGGCGA
AAAAGAGGGTTCTTGAACCTCTGGGCCTGGTTGAGGAACCTGTTAAGACGGCTCCGGGAAAAAAGA
GGCCGGTAGAGCACTCTCCTGTGGAGCCAGACTCCTCCTCGGGAACCGGAAAGGCGGGCCAGCAGC
CTGCAAGAAAAAGATTGAATTTTGGTCAGACTGGAGACGCAGACTCAGTACCTGACCCCCAGCCTC
TCGGACAGCCACCAGCAGCCCCCTCTGGTCTGGGAACTAATACGATGGCTACAGGCAGTGGCGCAC
CAATGGCAGACAATAACGAGGGCGCCGACGGAGTGGGTAATTCCTCGGGAAATTGGCATTGCGATT
CCACATGGATGGGCGACAGAGTCATCACCACCAGCACCCGAACCTGGGCCCTGCCCACCTACAACA
ACCACCTCTACAAACAAATTTCCAGCCAATCAGGAGCCTCGAACGACAATCACTACTTTGGCTACA
GCACCCCTTGGGGGTATTTTGACTTCAACAGATTCCACTGCCACTTTTCACCACGTGACTGGCAAA
GACTCATCAACAACAACTGGGGATTCCGACCCAAGAGACTCAACTTCAAGCTCTTTAACATTCAAG
TCAAAGAGGTCACGCAGAATGACGGTACGACGACGATTGCCAATAACCTTACCAGCACGGTTCAGG
TGTTTACTGACTCGGAGTACCAGCTCCCGTACGTCCTCGGCTCGGCGCATCAAGGATGCCTCCCGC
CGTTCCCAGCAGACGTCTTCATGGTGCCACAGTATGGATACCTCACCCTGAACAACGGGAGTCAGG
CAGTAGGACGCTCTTCATTTTACTGCCTGGAGTACTTTCCTTCTCAGATGCTGCGTACCGGAAACA
ACTTTACCTTCAGCTACACTTTTGAGGACGTTCCTTTCCACAGCAGCTACGCTCACAGCCAGAGTC
TGGACCGTCTCATGAATCCTCTCATCGACCAGTACCTGTATTACTTGAGCAGAACAAACACTCCAA
GTGGAACCACCACGCAGTCAAGGCTTCAGTTTTCTCAGGCCGGAGCGAGTGACATTCGGGACCAGT
CTAGGAACTGGCTTCCTGGACCCTGTTACCGCCAGCAGCGAGTATCAAAGACATCTGCGGATAACA
ACAACAGTGAATACTCGTGGACTGGAGCTACCAAGTACCACCTCAATGGCAGAGACTCTCTGGTGA
ATCCGGGCCCGGCCATGGCAAGCCACAAGGACGATGAAGAAAAGTTTTTTCCTCAGAGCGGGGTTC
TCATCTTTGGGAAGCAAGGCTCAGAGAAAACAAATGTGGACATTGAAAAGGTCATGATTACAGACG
AAGAGGAAATCAGGACAACCAATCCCGTGGCTACGGAGCAGTATGGTTCTGTATCTACCAACCTCC
AGAGAGGCAACAGACAAGCAGCTACCGCAGATGTCAACACACAAGGCGTTCTTCCAGGCATGGTCT
GGCAGGACAGAGATGTGTACCTTCAGGGGCCCATCTGGGCAAAGATTCCACACACGGACGGACATT
TTCACCCCTCTCCCCTCATGGGTGGATTCGGACTTAAACACCCTCCTCCACAGATTCTCATCAAGA
ACACCCCGGTACCTGCGAATCCTTCGACCACCTTCAGTGCGGCAAAGTTTGCTTCCTTCATCACAC
AGTACTCCACGGGACAGGTCAGCGTGGAGATCGAGTGGGAGCTGCAGAAGGAAAACAGCAAACGCT
GGAATCCCGAAATTCAGTACACTTCCAACTACAACAAGTCTGTTAATGTGGACTTTACTGTGGACA
CTAATGGCGTGTATTCAGAGCCTCGCCCCATTGGCACCAGATACCTGACTCGTAATCTGTAATTGC
TTGTTAATCAATAAACCGTTTAATTCGTTTCAGTTGAACTTTGGTCTCTGCGTATTTCTTTCTTAT
CTAGTTTCCATGGCTACGTAGATAAGTAGCATGGCGGGTTAATCATTAACTACATAAGGGCGAATT
CTGCAGATATCCATCACACTGGCGGCCGCTCGAGCATGCATCTAGAGCTAGCGAATTCGAATTTAA
ATCGGATCCGCGGCCGCAAGGATCTGCGATCGCTCCGGTGCCCGTCAGTGGGCAGAGCGCACATCG
CCCACAGTCCCCGAGAAGTTGGGGGGAGGGGTCGGCAATTGAACGGGTGCCTAGAGAAGGTGGCGC
GGGGTAAACTGGGAAAGTGATGTCGTGTACTGGCTCCGCCTTTTTCCCGAGGGTGGGGGAGAACCG
TATATAAGTGCAGTAGTCGCCGTGAACGTTCTTTTTCGCAACGGGTTTGCCGCCAGAACACAGCTG
AAGCTTCGAGGGGCTCGCATCTCTCCTTCACGCGCCCGCCGCCCTACCTGAGGCCGCCATCCACGC
CGGTTGAGTCGCGTTCTGCCGCCTCCCGCCTGTGGTGCCTCCTGAACTGCGTCCGCCGTCTAGGTA
AGTTTAAAGCTCAGGTCGAGACCGGGCCTTTGTCCGGCGCTCCCTTGGAGCCTACCTAGACTCAGC
CGGCTCTCCACGCTTTGCCTGACCCTGCTTGCTCAACTCTACGTCTTTGTTTCGTTTTCTGTTCTG
CGCCGTTACAGATCCAAGCTGTGACCGGCGCCTACGATATCGCCACCATGAAAACATTTAACATTT
CTCAACAGGATCTAGAATTAGTAGAAGTAGCGACAGAGAAGATTACAATGCTTTATGAGGATAATA
AACATCATGTGGGAGCGGCAATTCGTACGAAAACAGGAGAAATCATTTCGGCAGTACATATTGAAG
CGTATATAGGACGAGTAACTGTTTGTGCAGAAGCCATTGCGATTGGTAGTGCAGTTTCGAATGGAC
AAAAGGATTTTGACACGATTGTAGCTGTTAGACACCCTTATTCTGACGAAGTAGATAGAAGTATTC
GAGTGGTAAGTCCTTGTGGTATGTGTAGGGAGTTGATTTCAGACTATGCACCAGATTGTTTTGTGT
TAATAGAAATGAATGGCAAGTTAGTCAAAACTACGATTGAAGAACTCATTCCACTCAAATATACCC
GAAATTAAGGTACCTCGACAACCTTCCAAACTGAGTGCATGACCCGCAAGCCCGGTGCCTGAAATC
AACCTCTGGATTACAAAATTTGTGAAAGATTGACTGGTATTCTTAACTATGTTGCTCCTTTTACGC
TATGTGGATACGCTGCTTTAATGCCTTTGTATCATGCGTTAACTAAACTTGTTTATTGCAGCTTAT
AATGGTTACAAATAAAGCAATAGCATCACAAATTTCACAAATAAAGCATTTTTTTCACTGCATTCT
AGTTGTGGTTTGTCCAAACTCATCAATGTATCTTATCATGTCTGGAATTGACTCAAATGATGTCAA
TTAGTCTATCAGAAGCTATCTGGTCTCCCTTCCGGGGGACAAGACATCCCTGTTTAATATTTAAAC
AGCAGTGTTCCCAAACTGGGTTCTTATATCCCTTGCTCTGGTCAACCAGGTTGCAGGGTTTCCTGT
CCTCACAGGAACGAAGTCCCTAAAGAAACAGTGGCAGCCAGGTTTAGCCCCGGAATTGACTGGATT
CCTTTTTTAGGGCCCATTGGTATGGCTTTTTCCCCGTATCCCCCCAGGTGTCTGCAGGCTCAAAGA
GCAGCGAGAAGCGTTCAGAGGAAAGCGATCCCGTGCCACCTTCCCCGTGCCCGGGCTGTCCCCGCA
CGCTGCCGGCTCGGGGATGCGGGGGGAGCGCCGGACCGGAGCGGAGCCCCGGGCGGCTCGCTGCTG
CCCCCTAGCGGGGGAGGGACGTAATTACATCCCTGGGGGCTTTGGGGGGGGGCTGTCCCTGATATC
TATAACAAGAAAATATATATATAATAAGTTATCACGTAAGTAGAACATGAAATAACAATATAATTA
TCGTATGAGTTAAATCTTAAAAGTCACGTAAAAGATAATCATGCGTCATTTTGACTCACGCGGTCG
TTATAGTTCAAAATCAGTGACACTTACCGCATTGACAAGCACGCCTCACGGGAGCTCCAAGCGGCG
ACTGAGATGTCCTAAATGCACAGCGACGGATTCGCGCTATTTAGAAAGAGAGAGCAATATTTCAAG
AATGCATGCGTCAATTTTACGCAGACTATCTTTCTAGGGTTAATCTAGCTGCATCAGGATCATATC
GTCGGGTCTTTTTTCCGGCTCAGTCATCGCCCAAGCTGGCGCTATCTGGGCATCGGGGAGGAAGAA
GCCCGTGCCTTTTCCCGCGAGGTTGAAGCGGCATGGAAAGAGTTTGCCGAGGATGACTGCTGCTGC
ATTGACGTTGAGCGAAAACGCACGTTTACCATGATGATTCGGGAAGGTGTGGCCATGCACGCCTTT
AACGGTGAACTGTTCGTTCAGGCCACCTGGGATACCAGTTCGTCGCGGCTTTTCCGGACACAGTTC
CGGATGGTCAGCCCGAAGCGCATCAGCAACCCGAACAATACCGGCGACAGCCGGAACTGCCGTGCC
GGTGTGCAGATTAATGACAGCGGTGCGGCGCTGGGATATTACGTCAGCGAGGACGGGTATCCTGGC
TGGATGCCGCAGAAATGGACATGGATACCCCGTGAGTTACCCGGCGGGCGCGCTTGGCGTAATCAT
GGTCATAGCTGTTTCCTGTGTGAAATTGTTATCCGCTCACAATTCCACACAACATACGAGCCGGAA
GCATAAAGTGTAAAGCCTGGGGTGCCTAATGAGTGAGCTAACTCACATTAATTGCGTTGCGCTCAC
TGCCCGCTTTCCAGTCGGGAAACCTGTCGTGCCAGCTGCATTAATGAATCGGCCAACGCGCGGGGA
GAGGCGGTTTGCGTATTGGGCGCTCTTCCGCTTCCTCGCTCACTGACTCGCTGCGCTCGGTCGTTC
GGCTGCGGCGAGCGGTATCAGCTCACTCAAAGGCGGTAATACGGTTATCCACAGAATCAGGGGATA
ACGCAGGAAAGAACATGTGAGCAAAAGGCCAGCAAAAGGCCAGGAACCGTAAAAAGGCCGCGTTGC
TGGCGTTTTTCCATAGGCTCCGCCCCCCTGACGAGCATCACAAAAATCGACGCTCAAGTCAGAGGT
GGCGAAACCCGACAGGACTATAAAGATACCAGGCGTTTCCCCCTGGAAGCTCCCTCGTGCGCTCTC
CTGTTCCGACCCTGCCGCTTACCGGATACCTGTCCGCCTTTCTCCCTTCGGGAAGCGTGGCGCTTT
CTCATAGCTCACGCTGTAGGTATCTCAGTTCGGTGTAGGTCGTTCGCTCCAAGCTGGGCTGTGTGC
ACGAACCCCCCGTTCAGCCCGACCGCTGCGCCTTATCCGGTAACTATCGTCTTGAGTCCAACCCGG
TAAGACACGACTTATCGCCACTGGCAGCAGCCACTGGTAACAGGATTAGCAGAGCGAGGTATGTAG
GCGGTGCTACAGAGTTCTTGAAGTGGTGGCCTAACTACGGCTACACTAGAAGGACAGTATTTGGTA
TCTGCGCTCTGCTGAAGCCAGTTACCTTCGGAAAAAGAGTTGGTAGCTCTTGATCCGGCAAACAAA
CCACCGCTGGTAGCGGTGGTTTTTTTGTTTGCAAGCAGCAGATTACGCGCAGAAAAAAAGGATCTC
AAGAAGATCCTTTGATCTTTTCTACGGGGTCTGACGCTCAGTGGAACGAAAACTCACGTTAAGGGA
TTTTGGTCATGAGATTATCAAAAAGGATCTTCACCTAGATCCTTTTAAATTAAAAATGAAGTTTTA
AATCAATCTAAAGTATATATGAGTAAACTTGGTCTGACAGTTACCAATGCTTAATCAGTGAGGCAC
CTATCTCAGCGATCTGTCTATTTCGTTCATCCATAGTTGCCTGACTCCCCGTCGTGTAGATAACTA
CGATACGGGAGGGCTTACCATCTGGCCCCAGTGCTGCAATGATACCGCGAGACCCACGCTCACCGG
CTCCAGATTTATCAGCAATAAACCAGCCAGCCGGAAGGGCCGAGCGCAGAAGTGGTCCTGCAACTT
TATCCGCCTCCATCCAGTCTATTAATTGTTGCCGGGAAGCTAGAGTAAGTAGTTCGCCAGTTAATA
GTTTGCGCAACGTTGTTGCCATTGCTACAGGCATCGTGGTGTCACGCTCGTCGTTTGGTATGGCTT
CATTCAGCTCCGGTTCCCAACGATCAAGGCGAGTTACATGATCCCCCATGTTGTGCAAAAAAGCGG
TTAGCTCCTTCGGTCCTCCGATCGTTGTCAGAAGTAAGTTGGCCGCAGTGTTATCACTCATGGTTA
TGGCAGCACTGCATAATTCTCTTACTGTCATGCCATCCGTAAGATGCTTTTCTGTGACTGGTGAGT
ACTCAACCAAGTCATTCTGAGAATAGTGTATGCGGCGACCGAGTTGCTCTTGCCCGGCGTCAATAC
GGGATAATACCGCGCCACATAGCAGAACTTTAAAAGTGCTCATCATTGGAAAACGTTCTTCGGGGC
GAAAACTCTCAAGGATCTTACCGCTGTTGAGATCCAGTTCGATGTAACCCACTCGTGCACCCAACT
GATCTTCAGCATCTTTTACTTTCACCAGCGTTTCTGGGTGAGCAAAAACAGGAAGGCAAAATGCCG
CAAAAAAGGGAATAAGGGCGACACGGAAATGTTGAATACTCAT
SEQ ID NO: 9 (STXC0090)
ACTCTTCCTTTTTCAATATTATTGAAGCATTTATCAGGGTTATTGTCTCATGAGCGGATACATATT
TGAATGTATTTAGAAAAATAAACAAATAGGGGTTCCGCGCACATTTCCCCGAAAAGTGCCACCTAA
ATTGTAAGCGTTAATATTTTGTTAAAATTCGCGTTAAATTTTTGTTAAATCAGCTCATTTTTTAAC
CAATAGGCCGAAATCGGCAAAATCCCTTATAAATCAAAAGAATAGACCGAGATAGGGTTGAGTGTT
GTTCCAGTTTGGAACAAGAGTCCACTATTAAAGAACGTGGACTCCAACGTCAAAGGGCGAAAAACC
GTCTATCAGGGCGATGGCCCACTACGTGAACCATCACCCTAATCAAGTTTTTTGGGGTCGAGGTGC
CGTAAAGCACTAAATCGGAACCCTAAAGGGAGCCCCCGATTTAGAGCTTGACGGGGAAAGCCGGCG
AACGTGGCGAGAAAGGAAGGGAAGAAAGCGAAAGGAGCGGGCGCTAGGGCGCTGGCAAGTGTAGCG
GTCACGCTGCGCGTAACCACCACACCCGCCGCGCTTAATGCGCCGCTACAGGGCGCGTCCCATTCG
CCATTCAGGCTGCGCAACTGTTGGGAAGGGCGATCGGTGCGGGCCTCTTCGCTATTACGCCAGCTG
GCGAAAGGGGGATGTGCTGCAAGGCGATTAAGTTGGGTAACGCCAGGGTTTTCCCAGTCACGACGT
TGTAAAACGACGGCCAGTGAGCGCGCCTCGTTCATTCACGTTTTTGAACCCGTGGAGGACGGGCAG
ACTCGCGGTGCAAATGTGTTTTACAGCGTGATGGAGCAGATGAAGATGCTCGACACGCTGCAGAAC
ACGCAGCTAGATTAACCCTAGAAAGATAATCATATTGTGACGTACGTTAAAGATAATCATGTGTAA
AATTGACGCATGTGTTTTATCGGTCTGTATATCGAGGTTTATTTATTAATTTGAATAGATATTAAG
TTTTATTATATTTACACTTACATACTAATAATAAATTCAACAAACAATTTATTTATGTTTATTTAT
TTATTAAAAAAAACAAAAACTCAAAATTTCTTCTATAAAGTAACAAAACTTTTATGAGGGACAGCC
CCCCCCCAAAGCCCCCAGGGATGTAATTACGTCCCTCCCCCGCTAGGGGGCAGCAGCGAGCCGCCC
GGGGCTCCGCTCCGGTCCGGCGCTCCCCCCGCATCCCCGAGCCGGCAGCGTGCGGGGACAGCCCGG
GCACGGGGAAGGTGGCACGGGATCGCTTTCCTCTGAACGCTTCTCGCTGCTCTTTGAGCCTGCAGA
CACCTGGGGGGATACGGGGAAAAGGCCTCCACGGCCACTAGTCCATAGAGCCCACCGCATCCCCAG
CATGCCTGCTATTGTCTTCCCAATCCTCCCCCTTGCTGTCCTGCCCCACCCCACCCCCTAGAATAG
AATGACACCTACTCAGACAATGCGATGCAATTTCCTCATTTTATTAGGAAAGGACAGTGGGAGTGG
CACCTTCCAGGGTCAAGGAAGGCACGGGGGAGGGGCAAACAACAGATGGCTGGCAACTAGAAGGCA
CAGCTACATGGGGGTAGAGTCATAATCGTGCATCAGGATAGGGCGGTGGTGCTGCAGCAGCGCGCG
AATAAACTGCTGCCGCCGCCGCTCCGTCCTGCAGGAATACAACATGGCAGTGGTCTCCTCAGCGAT
GATTCGCACCGCCCGCAGCATGAGACGCCTTGTCCTCCGGGCACAGCAGCGCACCCTGATCTCACT
TAAATCAGCACAGTAACTGCAGCACAGCACCACAATATTGTTCAAAATCCCACAGTGCAAGGCGCT
GTATCCAAAGCTCATGGCGGGGACCACAGAACCCACGTGGCCATCATACCACAAGCGCAGGTAGAT
TAAGTGGCGACCCCTCATAAACACGCTGGACATAAACATTACCTCTTTTGGCATGTTGTAATTCAC
CACCTCCCGGTACCATATAAACCTCTGATTAAACATGGCGCCATCCACCACCATCCTAAACCAGCT
GGCCAAAACCTGCCCGCCGGCTATGCACTGCAGGGAACCGGGACTGGAACAATGACAGTGGAGAGC
CCAGGACTCGTAACCATGGATCATCATGCTCGTCATGATATCAATGTTGGCACAACACAGGCACAC
GTGCATACACTTCCTCAGGATTACAAGCTCCTCCCGCGTCAGAACCATATCCCAGGGAACAACCCA
TTCCTGAATCAGCGTAAATCCCACACTGCAGGGAAGACCTCGCACGTAACTCACGTTGTGCATTGT
CAAAGTGTTACATTCGGGCAGCAGCGGATGATCCTCCAGTATGGTAGCGCGGGTCTCTGTCTCAAA
AGGAGGTAGGCGATCCCTACTGTACGGAGTGCGCCGAGACAACCGAGATCGTGTTGGTCGTAGTGT
CATGCCAAATGGAACGCCGGACGTAGTCATGGTTGTGGCCATATTATCATCGTGTTTTTCAAAGGA
AAACCACGTCCCCGTGGTTCGGGGGGCCTAGACGTTTTTTTAACCTCGACTAAACACATGTAAAGC
ATGTGCACCGAGGCCCCAGATCAGATCCCATACAATGGGGTACCTTCTGGGCATCCTTCAGCCCCT
TGTTGAATACGCTTGAGGAGAGCCATTTGACTCTTTCCACAACTATCCAACTCACAACGTGGCACT
GGGGTTGTGCCGCCTTTGCAGGTGTATCTTATACACGTGGCTTTTGGCCGCAGAGGCACCTGTCGC
CAGGTGGGGGGTTCCGCTGCCTGCAAAGGGTCGCTACAGACGTTGTTTGTCTTCAAGAAGCTTCCA
GAGGAACTGCTTCCTTCACGACATTCAACAGACCTTGCATTCCTTTGGCGAGAGGGGAAAGACCCC
TAGGAATGCTCGTCAAGAAGACAGGGCCAGGTTTCCGGGCCCTCACATTGCCAAAAGACGGCAATA
TGGTGGAAAATAACATATAGACAAACGCACACCGGCCTTATTCCAAGCGGCTTCGGCCAGTAACGT
TAGGGGGGGGGGAGGGAGAGGGGCTTAAAAATCAAAGGGGTTCTGCCGCGCATCACTATGCGCCAC
TGGCAGGGACACGTTGCGATACTGGTGTTTAGTGCTCCACTTAAACTCAGGCACAACCATCCGCGG
CAGCTCGGTGAAGTTTTCACTCCACAGGCTGCGCACCATCACCAACGCGTTTAGCAGGTCGGGCGC
CGATATCTTGAAGTCGCAGTTGGGGCCTCCGCCCTGCGCGCGCGAGTTGCGATACACAGGGTTGCA
GCACTGGAACACTATCAGCGCCGGGTGGTGCACGCTGGCCAGCACGCTCTTGTCGGAGATCAGATC
CGCGTCCAGGTCCTCCGCGTTGCTCAGGGCGAACGGAGTCAACTTTGGTAGCTGCCTTCCCAAAAA
GGGTGCATGCCCAGGCTTTGAGTTGCACTCGCACCGTAGTGGCATCAGAAGGTGACCGTGCCCGGT
CTGGGCGTTAGGATACAGCGCCTGCATGAAAGCCTTGATCTGCTTAAAAGCCACCTGAGCCTTTGC
GCCTTCAGAGAAGAACATGCCGCAAGACTTGCCGGAAAACTGATTGGCCGGACAGGCCGCGTCATG
CACGCAGCACCTTGCGTCGGTGTTGGAGATCTGCACCACATTTCGGCCCCACCGGTTCTTCACGAT
CTTGGCCTTGCTAGACTGCTCCTTCAGCGCGCGCTGCCCGTTTTCGCTCGTCACATCCATTTCAAT
CACGTGCTCCTTATTTATCATAATGCTCCCGTGTAGACACTTAAGCTCGCCTTCGATCTCAGCGCA
GCGGTGCAGCCACAACGCGCAGCCCGTGGGCTCGTGGTGCTTGTAGGTTACCTCTGCAAACGACTG
CAGGTACGCCTGCAGGAATCGCCCCATCATCGTCACAAAGGTCTTGTTGCTGGTGAAGGTCAGCTG
CAACCCGCGGTGCTCCTCGTTTAGCCAGGTCTTGCATACGGCCGCCAGAGCTTCCACTTGGTCAGG
CAGTAGCTTGAAGTTTGCCTTTAGATCGTTATCCACGTGGTACTTGTCCATCAACGCGCGCGCAGC
CTCCATGCCCTTCTCCCACGCAGACACGATCGGCAGGCTCAGCGGGTTTATCACCGTGCTTTCACT
TTCCGCTTCACTGGACTCTTCCTTTTCCTCTTGCGTCCGCATACCCCGCGCCACTGGGTCGTCTTC
ATTCAGCCGCCGCACCGTGCGCTTACCTCCCTTGCCGTGCTTGATTAGCACCGGTGGGTTGCTGAA
ACCCACCATTTGTAGCGCCACATCTTCTCTTTCTTCCTCGCTGTCCACGATCACCTCTGGGGATGG
CGGGCGCTCGGGCTTGGGAGAGGGGCGCTTCTTTTTCTTTTTGGACGCAATGGCCAAATCCGCCGT
CGAGGTCGATGGCCGCGGGCTGGGTGTGCGCGGCACCAGCGCATCTTGTGACGAGTCTTCTTCGTC
CTCGGACTCGAGACGCCGCCTCAGCCGCTTTTTTGGGGGCGCGCGCTTGTCGTCATCGTCTTTGTA
GTCGGGAGGCGGCGGCGACGGCGACGGGGACGACACGTCCTCCATGGTTGGTGGACGTCGCGCCGC
ACCGCGTCCGCGCTCGGGGGTGGTTTCGCGCTGCTCCTCTTCCCGACTGGCCATGGTGGCCGAGGA
TAACTTCGTATATGGTTTCTTATACGAAGTTATGATCCAGACATGATAAGATACATTGATGAGTTT
GGACAAACCACAACTAGAATGCAGTGAAAAAAATGCTTTATTTGTGAAATTTGTGATGCTATTGCT
TTATTTGTAACCATTATAAGCTGCAATAAACAAGTTAACAACAACAATTGCATTCATTTTATGTTT
CAGGTTCAGGGGGAGGTGTGGGAGGTTTTTTAAAGCAAGTAAAACCTCTACAAATGTGGTATGGCT
GATTATGATCCTCTAGAGTCGCAGATCTGCTACGTATCAAGCTGTGGCAGGGAAACCCTCTGCCTC
CCCCGTGATGTAATACTTTTGCAAGGAATGCGATGAAGTAGAGCCCGCAGTGGCCAAGTGGCTTTG
GTCCGTCTCCTCCACGGATGCCCCTCCACGGCTAGTGGGCGCATGTAGGCGGTGGGCGTCCGCCGC
CTCCAGCAGCAGGTCATAGAGGGGCACCACGTTCTTGCACTTCATGCTGTACAGATGCTCCATGCC
TTTGTTACTCATGTGTCGGATGTGGGAGAGGATGAGGAGGAGCTGGGCCAGCCGCTGGTGCTGCTG
CTGCAGGGTCAGGCCTGCCTTGGCCATCAGGTGGATCAAAGTGTCTGTGATCTTGTCCAGGACTCG
GTGGATATGGTCCTTCTCTTCCAGAGACTTCAGGGTGCTGGACAGAAATGTGTACACTCCAGAATT
AAGCAAAATAATAGATTTGAGGCACACAAACTCCTCTCCCTGCAGATTCATCATGCGGAACCGAGA
TGATGTAGCCAGCAGCATGTCGAAGATCTCCACCATGCCCTCTACACATTTTCCCTGGTTCCTGTC
CAAGAGCAAGTTAGGAGCAAACAGTAGCTTCACTGGGTGCTCCATGGAGCGCCAGACGAGACCAAT
CATCAGGATCTCTAGCCAGGCACATTCTAGAAGGTGGACCTGATCATGGAGGGTCAAATCCACAAA
GCCTGGCACCCTCTTCGCCCAGTTGATCATGTGAACCAGCTCCCTGTCTGCCAGGTTGGTCAGTAA
GCCCATCATCGAAGCTTCACTGAAGGGTCTGGTAGGATCATACTCGGAATAGAGTATGGGGGGCTC
AGCATCCAACAAGGCACTGACCATCTGGTCGGCCGTCAGGGACAAGGCCAGGCTGTTCTTCTTAGA
GCGTTTGATCATGAGCGGGCTTGGCCAAAGGTTGGCAGCTCTCATGTCTCCAGCAGATGGCTCGAG
ATCGCCATCTTCCAGCAGGCGCACCATTGCCCCTGTTTCACTATCCAGGTTACGGATATAGTTCAT
GACAATATTTACATTGGTCCAGCCACCAGCTTGCATGATCTCCGGTATTGAAACTCCAGCGCGGGC
CATATCTCGCGCGGCTCCGACACGGGCACTGTGTCCAGACCAGGCCAGGTATCTCTGACCAGAGTC
ATCCTAAAATACACAAACAATTAGAATCAGTAGTTTAACACATTATACACTTAAAAATTTTATATT
TACCTTAGCGCCGTAAATCAATCGATGAGTTGCTTCAAAAATCCCTTCCAGGGCGCGAGTTGATAG
CTGGCTGGTGGCAGATGGCGCGGCAACACCATTTTTTCTGACCCGGCAAAACAGGTAGTTATTCGG
ATCATCAGCTACACCAGAGACGGAAATCCATCGCTCGACCAGTTTAGTGACTCCCAGGCTAAGTGC
CTTCTCTACACCTGCGGTGCTAACCAGCGTTTTCGTTCTGCCAATATGGATTAACATTCTCCCACC
GTCAGTACGTGAGATATCTTTAACCCTGATCCTGGCAATTTCGGCTATACGTAACAGGGTGTTATA
AGCAATCCCCAGAAATGCCAGATTACGTATATCCTGGCAGCGATCGCTATTTTCCATGAGTGAACG
GACTTGGTCGAAATCAGTGCGTTCGAACGCTAGAGCCTGTTTTGCACGTTCACCGGCATCAACGTT
TTCTTTTCGGATCCGCCGCATAACCAGTGAAACAGCATTGCTGTCACTTGGTCGTGGCAGCCCGGA
CCGACGATGAAGCATGTTTAGCTGGCCCAAATGTTGCTGGATAGTTTTTACTGCCAGACCGCGCGC
TTGAAGATATAGAAGATAATCGCGAACATCTTCAGGTTCTGCGGGAAACCATTTCCGGTTATTCAA
CTTGCACCATGCCGCCCACGACCGGCAAACGGACAGAAGCATTTTCCAGGTATGCTCAGAAAACGC
CTGGCGATCCCTGAACATGTCCATCAGGTTCTTGCGAACCTCATCACTCGTTGCATCGACCGGTAA
TGCAGGCAAATTTTGGTGTACGGTCAGTAAATTGGACATGGTGGCTACGTAATAACTTCGTATATG
GTTTCTTATACGAAGTTATGCGGCCGCTTTACGAGGGTAGGAAGTGGTACGGAAAGTTGGTATAAG
ACAAAAGTGTTGTGGAATTGCTCCAGGCGATCTGACGGTTCACTAAACGAGCTCTGCTTTTATAGG
CGCCCACCGTACACGCCTAAAGCTTATACGTTCTCTATCACTGATAGGGAGTAAACTGGATATACG
TTCTCTATCACTGATAGGGAGTAAACTGTAGATACGTTCTCTATCACTGATAGGGAGTAAACTGGT
CATACGTTCTCTATCACTGATAGGGAGTAAACTCCTTATACGTTCTCTATCACTGATAGGGAGTAA
AGTCTGCATACGTTCTCTATCACTGATAGGGAGTAAACTCTTCATACGTTCTCTATCACTGATAGG
GAGTAAACTCGCGGCCGCAGAGAAATGTTCTGGCACCTGCACTTGCACTGGGGACAGCCTATTTTG
CTAGTTTGTTTTGTTTCGTTTTGTTTTGATGGAGAGCGTATGTTAGTACTATCGATTCACACAAAA
AACCAACACACAGATGTAATGAAAATAAAGATATTTTATTGGATCTGCGATCGCTCCGGTGCCCGT
CAGTGGGCAGAGCGCACATCGCCCACAGTCCCCGAGAAGTTGGGGGGAGGGGTCGGCAATTGAACG
GGTGCCTAGAGAAGGTGGCGCGGGGTAAACTGGGAAAGTGATGTCGTGTACTGGCTCCGCCTTTTT
CCCGAGGGTGGGGGAGAACCGTATGTAAGTGCAGTAGTCGCCGTGAACGTTCTTTTTCGCAACGGG
TTTGCCGCCAGAACACAGCTGAAGCTTCGAGGGGCTCGCATCTCTCCTTCACGCGCCCGCCGCCCT
ACCTGAGGCCGCCATCCACGCCGGTTGAGTCGCGTTCTGCCGCCTCCCGCCTGTGGTGCCTCCTGA
ACTGCGTCCGCCGTCTAGGTAAGTTTAAAGCTCAGGTCGAGACCGGGCCTTTGTCCGGCGCTCCCT
TGGAGCCTACCTAGACTCAGCCGGCTCTCCACGCTTTGCCTGACCCTGCTTGCTCAACTCTACGTC
TTTGTTTCGTTTTCTGTTCTGCGCCGTTACAGATCCAAGCTGTGACCGGCGCCTACGCTAGCGGAT
CCGCCGCCACCATGTCTAGACTGGACAAGAGCAAAGTCATAAACTCTGCTCTGGAATTACTCAATG
GAGTCGGTATCGAAGGCCTGACGACAAGGAAACTCGCTCAAAAGCTGGGAGTTGAGCAGCCTACCC
TGTACTGGCACGTGAAGAACAAGCGGGCCCTGCTCGATGCCCTGCCAATCGAGATGCTGGACAGGC
ATCATACCCACTCCTGCCCCCTGGAAGGCGAGTCATGGCAAGACTTTCTGCGGAACAACGCCAAGT
CATACCGCTGTGCTCTTCTCTCACATCGCGACGGGGCTAAAGTGCATCTCGGCACCCGCCCAACAG
AGAAACAGTACGAAACCCTGGAAAATCAGCTCGCGTTCCTGTGTCAGCAAGGCTTCTCCCTGGAGA
ACGCACTGTACGCTCTGTCCGCCGTGGGCCACTTTACACTGGGCTGCGTATTGGAGGAACAGGAGC
ATCAAGTAGCAAAAGAGGAAAGAGAGACACCTACCACCGATTCTATGCCCCCACTTCTGAAACAAG
CAATTGAGCTGTTCGACCGGCAGGGAGCCGAACCTGCCTTCCTTTTCGGCCTGGAACTAATCATAT
GTGGCCTGGAGAAACAGCTAAAGTGCGAAAGCGGCGGGCCGACCGACGCCCTTGACGATTTTGACT
TAGACATGCTCCCAGCCGATGCCCTTGACGACTTTGACCTTGATATGCTGCCTGCTGACGCTCTTG
ACGATTTTGACCTTGACATGCTCCCCGGGTGAACCGGTCGCTGATCAGCCTCGACTGTGCCTTCTA
GTTGCCAGCCATCTGTTGTTTGCCCCTCCCCCGTGCCTTCCTTGACCCTGGAAGGTGCCACTCCCA
CTGTCCTTTCCTAATAAAATGAGGAAATTGCATCGCATTGTCTGAGTAGGTGTCATTCTATTCTGG
GGGGTGGGGTGGGGCAGGACAGCAAGGGGGAGGATTGGGAAGACAATAGCAGGCATGCTGGGGATG
CGGTGGGCTCTATGGCTTCTGAGGCGGAAAGAACCAGCTGGGGCTCGACTAGAGCTTGCGGAACCC
TTAGAGGGCCTATTTCCCATGATTCCTTCATATTTGCATATACGATACAAGGCTGTTAGAGAGATA
ATTAGAATTAATTTGACTGTAAACACAAAGATATTAGTACAAAATAATAACTTCGTATAATGTATG
CTATACGAAGTTATCAGACATGATAAGATACATTGATGAGTTTGGACAAACCACAACTAGAATGCA
GTGAAAAAAATGCTTTATTTGTGAAATTTGTGATGCTATTGCTTTATTTGTAACCATTATAAGCTG
CAATAAACAAGGTACCTCAAGCGCCGGGTTTTCGCGTCATGCACCACGTCCGTGGGCCCTCGGGTA
CTTCAACGTCAGCAGTAACTGTAAATCCGAGCCGTTCATAGAAGGGCAAATTCCTTGGCGCTGACG
TTTCAAGAAAGGCTGGCACTCCGGCTCGTTCTGCGGCTTCTACTCCGGGCAATACCACCGCGGAAC
CAAGGCCCTTTCCCTGATGATCGGGGCTAACGCCCACAGTAGCGAGGAACCAAGCTGGTTCTTTAG
GGCGGTGAGGGGCGAGGAGTCCTTCCATTTGTTGCTGAGCCGCGAGACGAGAGCCACTAAGCTCAG
CCATTCGGGGACCAATTTCTGCAAATACAGCCCCGGCCTCAACGCTCTCCGGAGTCGTCCACACTG
CCACTGCAGCCCCGTCGTCGGCGACCCAAACTTTACCGATGTCCAATCCTACCCTGGTCAAAAAAA
GTTCTTGCAATTCTGTAACCCGTTCAATATGTCTATCAGGATCAACTGTGTGGCGTGTAGCGGGAT
AATCCGCGAAAGCGGCAGCCAATGTTCTCACGGCCCTAGGGACGTCGTCTCGAGTTGCCAGTCTGA
CAGTAGGTTTATATTCTGTCATAGGTCCAGGGTTCTCCTCCACGTCTCCAGCCTGCTTCAGCAGGC
TGAAGTTAGTAGCTCCGGATCCTTTACCTCCATCACCAGCGCCACCAGTAGAGTATCTGGCCACAG
CCACCTCGTGCTGCTCGACGTAGGTCTCATCGTCGGCCTCCTTGATTCTTTCCAGTCTGTGGTCCA
CGTTGTAGACGCCGGGCATCTTGAGGTTCGTAGCGGGTTTCTTGGATCTGTATGTGGTCTCAAGGT
TGCAGATCAGGTGGCCCCCGCCCACGAGCTTCAGGGCCATGTCACATGCGCCTTCCAGGCCGCCGT
CAGCGGGGTACATCGTCTCGGTGGAGGCCTCCCAGCCGAGTGTTTTCTTCTGCATCACAGGGCCGT
TGGCTGGGAAGTTCACCCCTCTAACCTTGACGTTGTAGATGAGGCAGCCGTCCTGGAGGCTGGTGT
CCTGGGTAGCGGTCAGCACGCCCCCGTCTTCGTATGTGGTGACTCTCTCCCATGTGAAGCCCTCAG
GGAAGGACTGCTTAAAGAAGTCGGGGATGCCCGGAGGGTGCTTGATGAAGGTTCTGCTGCCGTACA
TGAAGCTGGTAGCCAGGATGTCGAAGGCGAAGGGGAGAGGGCCGCCCTCGACGACCTTGATTCTCA
TGGTCTGGGTGCCCTCGTAGGGCTTGCCTTCGCCCTCGGATGTGCACTTGAAGTGGTGGTTGTTCA
CGGTGCCCTCCATGTACAGCTTCATGGGCATGTTCTCCTTAATCAGCTCGCTCACGGTGGCGGCGA
ATTCCGAAAGGCCCGGAGATGAGGAAGAGGAGAACAGCGCGGCAGACGTGCGCTTTTGAAGCGTGC
AGAATGCCGGGCCTCCGGAGGACCTTCGGGCGCCCGCCCCGCCCCTGAGCCCGCCCCTGAGCCCGC
CCCCGGACCCACCCCTTCCCAGCCTCTGAGCCCAGAAAGCGAAGGAGCAAAGCTGCTATTGGCCGC
TGCCCCAAAGGCCTACCCGCTTCCATTGCTCAGCGGTGCTGTCCATCTGCACGAGACTAGTGAGAC
GTGCTACTTCCATTTGTCACGTCCTGCACGACGCGAGCTGCGGGGGGGGGGGGAACTTCCTGACTA
GGGGAGGAGTAGAAGGTGGCGCGAAGGGGCCACCAAAGAACGGAGCCGGTTGGCGCCTACCGGTGG
ATGTGGAATGTGTGCGAGGCCAGAGGCCACTTGTGTAGCGCCAAGTGCCCAGCGGGGCTGCTAAAG
CGCATGCTCCAGACTGCCTTGGGAAAAGCGCTCCCCTACCCATAACTTCGTATAATGTATGCTATA
CGAAGTTATTTTGCAGTTTTAAAATTATGTTTTAAAATGGACTATCATATGCTTACCGTAACTTGA
AAGTATTTCGATTTCTTGGCTTTATATATCTTGTGGAAAGGACGAAACACCGGGCACTCTTCCGTG
ATCTGGTGGATAAATTCGCAAGGGTATCATGGCGGACGACCGGGATTCGAACCCCGGATCCGGCCG
TCCGCCGTGATCCATGCGGTTACCGCCCGCGTGTCGAACCCAGGTGTGCGACGTCAGACAACGGGG
GAGCGCTCCTTTTTGGGCCCATTGGTATGGCTTTTTCCCCGTATCCCCCCAGGTGTCTGCAGGCTC
AAAGAGCAGCGAGAAGCGTTCAGAGGAAAGCGATCCCGTGCCACCTTCCCCGTGCCCGGGCTGTCC
CCGCACGCTGCCGGCTCGGGGATGCGGGGGGAGCGCCGGACCGGAGCGGAGCCCCGGGCGGCTCGC
TGCTGCCCCCTAGCGGGGGAGGGACGTAATTACATCCCTGGGGGCTTTGGGGGGGGGCTGTCCCTG
ATATCTATAACAAGAAAATATATATATAATAAGTTATCACGTAAGTAGAACATGAAATAACAATAT
AATTATCGTATGAGTTAAATCTTAAAAGTCACGTAAAAGATAATCATGCGTCATTTTGACTCACGC
GGTCGTTATAGTTCAAAATCAGTGACACTTACCGCATTGACAAGCACGCCTCACGGGAGCTCCAAG
CGGCGACTGAGATGTCCTAAATGCACAGCGACGGATTCGCGCTATTTAGAAAGAGAGAGCAATATT
TCAAGAATGCATGCGTCAATTTTACGCAGACTATCTTTCTAGGGTTAATCTAGCTGCATCAGGATC
ATATCGTCGGGTCTTTTTTCCGGCTCAGTCATCGCCCAAGCTGGCGCTATCTGGGCATCGGGGAGG
AAGAAGCCCGTGCCTTTTCCCGCGAGGTTGAAGCGGCATGGAAAGAGTTTGCCGAGGATGACTGCT
GCTGCATTGACGTTGAGCGAAAACGCACGTTTACCATGATGATTCGGGAAGGTGTGGCCATGCACG
CCTTTAACGGTGAACTGTTCGTTCAGGCCACCTGGGATACCAGTTCGTCGCGGCTTTTCCGGACAC
AGTTCCGGATGGTCAGCCCGAAGCGCATCAGCAACCCGAACAATACCGGCGACAGCCGGAACTGCC
GTGCCGGTGTGCAGATTAATGACAGCGGTGCGGCGCTGGGATATTACGTCAGCGAGGACGGGTATC
CTGGCTGGATGCCGCAGAAATGGACATGGATACCCCGTGAGTTACCCGGCGGGCGCGCTTGGCGTA
ATCATGGTCATAGCTGTTTCCTGTGTGAAATTGTTATCCGCTCACAATTCCACACAACATACGAGC
CGGAAGCATAAAGTGTAAAGCCTGGGGTGCCTAATGAGTGAGCTAACTCACATTAATTGCGTTGCG
CTCACTGCCCGCTTTCCAGTCGGGAAACCTGTCGTGCCAGCTGCATTAATGAATCGGCCAACGCGC
GGGGAGAGGCGGTTTGCGTATTGGGCGCTCTTCCGCTTCCTCGCTCACTGACTCGCTGCGCTCGGT
CGTTCGGCTGCGGCGAGCGGTATCAGCTCACTCAAAGGCGGTAATACGGTTATCCACAGAATCAGG
GGATAACGCAGGAAAGAACATGTGAGCAAAAGGCCAGCAAAAGGCCAGGAACCGTAAAAAGGCCGC
GTTGCTGGCGTTTTTCCATAGGCTCCGCCCCCCTGACGAGCATCACAAAAATCGACGCTCAAGTCA
GAGGTGGCGAAACCCGACAGGACTATAAAGATACCAGGCGTTTCCCCCTGGAAGCTCCCTCGTGCG
CTCTCCTGTTCCGACCCTGCCGCTTACCGGATACCTGTCCGCCTTTCTCCCTTCGGGAAGCGTGGC
GCTTTCTCATAGCTCACGCTGTAGGTATCTCAGTTCGGTGTAGGTCGTTCGCTCCAAGCTGGGCTG
TGTGCACGAACCCCCCGTTCAGCCCGACCGCTGCGCCTTATCCGGTAACTATCGTCTTGAGTCCAA
CCCGGTAAGACACGACTTATCGCCACTGGCAGCAGCCACTGGTAACAGGATTAGCAGAGCGAGGTA
TGTAGGCGGTGCTACAGAGTTCTTGAAGTGGTGGCCTAACTACGGCTACACTAGAAGGACAGTATT
TGGTATCTGCGCTCTGCTGAAGCCAGTTACCTTCGGAAAAAGAGTTGGTAGCTCTTGATCCGGCAA
ACAAACCACCGCTGGTAGCGGTGGTTTTTTTGTTTGCAAGCAGCAGATTACGCGCAGAAAAAAAGG
ATCTCAAGAAGATCCTTTGATCTTTTCTACGGGGTCTGACGCTCAGTGGAACGAAAACTCACGTTA
AGGGATTTTGGTCATGAGATTATCAAAAAGGATCTTCACCTAGATCCTTTTAAATTAAAAATGAAG
TTTTAAATCAATCTAAAGTATATATGAGTAAACTTGGTCTGACAGTTACCAATGCTTAATCAGTGA
GGCACCTATCTCAGCGATCTGTCTATTTCGTTCATCCATAGTTGCCTGACTCCCCGTCGTGTAGAT
AACTACGATACGGGAGGGCTTACCATCTGGCCCCAGTGCTGCAATGATACCGCGAGACCCACGCTC
ACCGGCTCCAGATTTATCAGCAATAAACCAGCCAGCCGGAAGGGCCGAGCGCAGAAGTGGTCCTGC
AACTTTATCCGCCTCCATCCAGTCTATTAATTGTTGCCGGGAAGCTAGAGTAAGTAGTTCGCCAGT
TAATAGTTTGCGCAACGTTGTTGCCATTGCTACAGGCATCGTGGTGTCACGCTCGTCGTTTGGTAT
GGCTTCATTCAGCTCCGGTTCCCAACGATCAAGGCGAGTTACATGATCCCCCATGTTGTGCAAAAA
AGCGGTTAGCTCCTTCGGTCCTCCGATCGTTGTCAGAAGTAAGTTGGCCGCAGTGTTATCACTCAT
GGTTATGGCAGCACTGCATAATTCTCTTACTGTCATGCCATCCGTAAGATGCTTTTCTGTGACTGG
TGAGTACTCAACCAAGTCATTCTGAGAATAGTGTATGCGGCGACCGAGTTGCTCTTGCCCGGCGTC
AATACGGGATAATACCGCGCCACATAGCAGAACTTTAAAAGTGCTCATCATTGGAAAACGTTCTTC
GGGGCGAAAACTCTCAAGGATCTTACCGCTGTTGAGATCCAGTTCGATGTAACCCACTCGTGCACC
CAACTGATCTTCAGCATCTTTTACTTTCACCAGCGTTTCTGGGTGAGCAAAAACAGGAAGGCAAAA
TGCCGCAAAAAAGGGAATAAGGGCGACACGGAAATGTTGAATACTCAT
SEQ ID NO: 10 (STXC0110)
ACTCTTCCTTTTTCAATATTATTGAAGCATTTATCAGGGTTATTGTCTCATGAGCGGATACATATT
TGAATGTATTTAGAAAAATAAACAAATAGGGGTTCCGCGCACATTTCCCCGAAAAGTGCCACCTAA
ATTGTAAGCGTTAATATTTTGTTAAAATTCGCGTTAAATTTTTGTTAAATCAGCTCATTTTTTAAC
CAATAGGCCGAAATCGGCAAAATCCCTTATAAATCAAAAGAATAGACCGAGATAGGGTTGAGTGTT
GTTCCAGTTTGGAACAAGAGTCCACTATTAAAGAACGTGGACTCCAACGTCAAAGGGCGAAAAACC
GTCTATCAGGGCGATGGCCCACTACGTGAACCATCACCCTAATCAAGTTTTTTGGGGTCGAGGTGC
CGTAAAGCACTAAATCGGAACCCTAAAGGGAGCCCCCGATTTAGAGCTTGACGGGGAAAGCCGGCG
AACGTGGCGAGAAAGGAAGGGAAGAAAGCGAAAGGAGCGGGCGCTAGGGCGCTGGCAAGTGTAGCG
GTCACGCTGCGCGTAACCACCACACCCGCCGCGCTTAATGCGCCGCTACAGGGCGCGTCCCATTCG
CCATTCAGGCTGCGCAACTGTTGGGAAGGGCGATCGGTGCGGGCCTCTTCGCTATTACGCCAGCTG
GCGAAAGGGGGATGTGCTGCAAGGCGATTAAGTTGGGTAACGCCAGGGTTTTCCCAGTCACGACGT
TGTAAAACGACGGCCAGTGAGCGCGCCTCGTTCATTCACGTTTTTGAACCCGTGGAGGACGGGCAG
ACTCGCGGTGCAAATGTGTTTTACAGCGTGATGGAGCAGATGAAGATGCTCGACACGCTGCAGAAC
ACGCAGCTAGATTAACCCTAGAAAGATAATCATATTGTGACGTACGTTAAAGATAATCATGTGTAA
AATTGACGCATGTGTTTTATCGGTCTGTATATCGAGGTTTATTTATTAATTTGAATAGATATTAAG
TTTTATTATATTTACACTTACATACTAATAATAAATTCAACAAACAATTTATTTATGTTTATTTAT
TTATTAAAAAAAACAAAAACTCAAAATTTCTTCTATAAAGTAACAAAACTTTTATGAGGGACAGCC
CCCCCCCAAAGCCCCCAGGGATGTAATTACGTCCCTCCCCCGCTAGGGGGCAGCAGCGAGCCGCCC
GGGGCTCCGCTCCGGTCCGGCGCTCCCCCCGCATCCCCGAGCCGGCAGCGTGCGGGGACAGCCCGG
GCACGGGGAAGGTGGCACGGGATCGCTTTCCTCTGAACGCTTCTCGCTGCTCTTTGAGCCTGCAGA
CACCTGGGGGGATACGGGGAAAAGGCCTCCACGGCCACTAGTCCATAGAGCCCACCGCATCCCCAG
CATGCCTGCTATTGTCTTCCCAATCCTCCCCCTTGCTGTCCTGCCCCACCCCACCCCCTAGAATAG
AATGACACCTACTCAGACAATGCGATGCAATTTCCTCATTTTATTAGGAAAGGACAGTGGGAGTGG
CACCTTCCAGGGTCAAGGAAGGCACGGGGGAGGGGCAAACAACAGATGGCTGGCAACTAGAAGGCA
CAGCTACATGGGGGTAGAGTCATAATCGTGCATCAGGATAGGGCGGTGGTGCTGCAGCAGCGCGCG
AATAAACTGCTGCCGCCGCCGCTCCGTCCTGCAGGAATACAACATGGCAGTGGTCTCCTCAGCGAT
GATTCGCACCGCCCGCAGCATGAGACGCCTTGTCCTCCGGGCACAGCAGCGCACCCTGATCTCACT
TAAATCAGCACAGTAACTGCAGCACAGCACCACAATATTGTTCAAAATCCCACAGTGCAAGGCGCT
GTATCCAAAGCTCATGGCGGGGACCACAGAACCCACGTGGCCATCATACCACAAGCGCAGGTAGAT
TAAGTGGCGACCCCTCATAAACACGCTGGACATAAACATTACCTCTTTTGGCATGTTGTAATTCAC
CACCTCCCGGTACCATATAAACCTCTGATTAAACATGGCGCCATCCACCACCATCCTAAACCAGCT
GGCCAAAACCTGCCCGCCGGCTATGCACTGCAGGGAACCGGGACTGGAACAATGACAGTGGAGAGC
CCAGGACTCGTAACCATGGATCATCATGCTCGTCATGATATCAATGTTGGCACAACACAGGCACAC
GTGCATACACTTCCTCAGGATTACAAGCTCCTCCCGCGTCAGAACCATATCCCAGGGAACAACCCA
TTCCTGAATCAGCGTAAATCCCACACTGCAGGGAAGACCTCGCACGTAACTCACGTTGTGCATTGT
CAAAGTGTTACATTCGGGCAGCAGCGGATGATCCTCCAGTATGGTAGCGCGGGTCTCTGTCTCAAA
AGGAGGTAGGCGATCCCTACTGTACGGAGTGCGCCGAGACAACCGAGATCGTGTTGGTCGTAGTGT
CATGCCAAATGGAACGCCGGACGTAGTCATGGTTGTGGCCATATTATCATCGTGTTTTTCAAAGGA
AAACCACGTCCCCGTGGTTCGGGGGGCCTAGACGTTTTTTTAACCTCGACTAAACACATGTAAAGC
ATGTGCACCGAGGCCCCAGATCAGATCCCATACAATGGGGTACCTTCTGGGCATCCTTCAGCCCCT
TGTTGAATACGCTTGAGGAGAGCCATTTGACTCTTTCCACAACTATCCAACTCACAACGTGGCACT
GGGGTTGTGCCGCCTTTGCAGGTGTATCTTATACACGTGGCTTTTGGCCGCAGAGGCACCTGTCGC
CAGGTGGGGGGTTCCGCTGCCTGCAAAGGGTCGCTACAGACGTTGTTTGTCTTCAAGAAGCTTCCA
GAGGAACTGCTTCCTTCACGACATTCAACAGACCTTGCATTCCTTTGGCGAGAGGGGAAAGACCCC
TAGGAATGCTCGTCAAGAAGACAGGGCCAGGTTTCCGGGCCCTCACATTGCCAAAAGACGGCAATA
TGGTGGAAAATAACATATAGACAAACGCACACCGGCCTTATTCCAAGCGGCTTCGGCCAGTAACGT
TAGGGGGGGGGGAGGGAGAGGGGCTTAAAAATCAAAGGGGTTCTGCCGCGCATCACTATGCGCCAC
TGGCAGGGACACGTTGCGATACTGGTGTTTAGTGCTCCACTTAAACTCAGGCACAACCATCCGCGG
CAGCTCGGTGAAGTTTTCACTCCACAGGCTGCGCACCATCACCAACGCGTTTAGCAGGTCGGGCGC
CGATATCTTGAAGTCGCAGTTGGGGCCTCCGCCCTGCGCGCGCGAGTTGCGATACACAGGGTTGCA
GCACTGGAACACTATCAGCGCCGGGTGGTGCACGCTGGCCAGCACGCTCTTGTCGGAGATCAGATC
CGCGTCCAGGTCCTCCGCGTTGCTCAGGGCGAACGGAGTCAACTTTGGTAGCTGCCTTCCCAAAAA
GGGTGCATGCCCAGGCTTTGAGTTGCACTCGCACCGTAGTGGCATCAGAAGGTGACCGTGCCCGGT
CTGGGCGTTAGGATACAGCGCCTGCATGAAAGCCTTGATCTGCTTAAAAGCCACCTGAGCCTTTGC
GCCTTCAGAGAAGAACATGCCGCAAGACTTGCCGGAAAACTGATTGGCCGGACAGGCCGCGTCATG
CACGCAGCACCTTGCGTCGGTGTTGGAGATCTGCACCACATTTCGGCCCCACCGGTTCTTCACGAT
CTTGGCCTTGCTAGACTGCTCCTTCAGCGCGCGCTGCCCGTTTTCGCTCGTCACATCCATTTCAAT
CACGTGCTCCTTATTTATCATAATGCTCCCGTGTAGACACTTAAGCTCGCCTTCGATCTCAGCGCA
GCGGTGCAGCCACAACGCGCAGCCCGTGGGCTCGTGGTGCTTGTAGGTTACCTCTGCAAACGACTG
CAGGTACGCCTGCAGGAATCGCCCCATCATCGTCACAAAGGTCTTGTTGCTGGTGAAGGTCAGCTG
CAACCCGCGGTGCTCCTCGTTTAGCCAGGTCTTGCATACGGCCGCCAGAGCTTCCACTTGGTCAGG
CAGTAGCTTGAAGTTTGCCTTTAGATCGTTATCCACGTGGTACTTGTCCATCAACGCGCGCGCAGC
CTCCATGCCCTTCTCCCACGCAGACACGATCGGCAGGCTCAGCGGGTTTATCACCGTGCTTTCACT
TTCCGCTTCACTGGACTCTTCCTTTTCCTCTTGCGTCCGCATACCCCGCGCCACTGGGTCGTCTTC
ATTCAGCCGCCGCACCGTGCGCTTACCTCCCTTGCCGTGCTTGATTAGCACCGGTGGGTTGCTGAA
ACCCACCATTTGTAGCGCCACATCTTCTCTTTCTTCCTCGCTGTCCACGATCACCTCTGGGGATGG
CGGGCGCTCGGGCTTGGGAGAGGGGCGCTTCTTTTTCTTTTTGGACGCAATGGCCAAATCCGCCGT
CGAGGTCGATGGCCGCGGGCTGGGTGTGCGCGGCACCAGCGCATCTTGTGACGAGTCTTCTTCGTC
CTCGGACTCGAGACGCCGCCTCAGCCGCTTTTTTGGGGGCGCGCGCTTGTCGTCATCGTCTTTGTA
GTCGGGAGGCGGCGGCGACGGCGACGGGGACGACACGTCCTCCATGGTTGGTGGACGTCGCGCCGC
ACCGCGTCCGCGCTCGGGGGTGGTTTCGCGCTGCTCCTCTTCCCGACTGGCCATGGTGGCCGAGGA
TAACTTCGTATATGGTTTCTTATACGAAGTTATGATCCAGACATGATAAGATACATTGATGAGTTT
GGACAAACCACAACTAGAATGCAGTGAAAAAAATGCTTTATTTGTGAAATTTGTGATGCTATTGCT
TTATTTGTAACCATTATAAGCTGCAATAAACAAGTTAACAACAACAATTGCATTCATTTTATGTTT
CAGGTTCAGGGGGAGGTGTGGGAGGTTTTTTAAAGCAAGTAAAACCTCTACAAATGTGGTATGGCT
GATTATGATCCTCTAGAGTCGCAGATCTGCTACGTATCAAGCTGTGGCAGGGAAACCCTCTGCCTC
CCCCGTGATGTAATACTTTTGCAAGGAATGCGATGAAGTAGAGCCCGCAGTGGCCAAGTGGCTTTG
GTCCGTCTCCTCCACGGATGCCCCTCCACGGCTAGTGGGCGCATGTAGGCGGTGGGCGTCCGCCGC
CTCCAGCAGCAGGTCATAGAGGGGCACCACGTTCTTGCACTTCATGCTGTACAGATGCTCCATGCC
TTTGTTACTCATGTGTCGGATGTGGGAGAGGATGAGGAGGAGCTGGGCCAGCCGCTGGTGCTGCTG
CTGCAGGGTCAGGCCTGCCTTGGCCATCAGGTGGATCAAAGTGTCTGTGATCTTGTCCAGGACTCG
GTGGATATGGTCCTTCTCTTCCAGAGACTTCAGGGTGCTGGACAGAAATGTGTACACTCCAGAATT
AAGCAAAATAATAGATTTGAGGCACACAAACTCCTCTCCCTGCAGATTCATCATGCGGAACCGAGA
TGATGTAGCCAGCAGCATGTCGAAGATCTCCACCATGCCCTCTACACATTTTCCCTGGTTCCTGTC
CAAGAGCAAGTTAGGAGCAAACAGTAGCTTCACTGGGTGCTCCATGGAGCGCCAGACGAGACCAAT
CATCAGGATCTCTAGCCAGGCACATTCTAGAAGGTGGACCTGATCATGGAGGGTCAAATCCACAAA
GCCTGGCACCCTCTTCGCCCAGTTGATCATGTGAACCAGCTCCCTGTCTGCCAGGTTGGTCAGTAA
GCCCATCATCGAAGCTTCACTGAAGGGTCTGGTAGGATCATACTCGGAATAGAGTATGGGGGGCTC
AGCATCCAACAAGGCACTGACCATCTGGTCGGCCGTCAGGGACAAGGCCAGGCTGTTCTTCTTAGA
GCGTTTGATCATGAGCGGGCTTGGCCAAAGGTTGGCAGCTCTCATGTCTCCAGCAGATGGCTCGAG
ATCGCCATCTTCCAGCAGGCGCACCATTGCCCCTGTTTCACTATCCAGGTTACGGATATAGTTCAT
GACAATATTTACATTGGTCCAGCCACCAGCTTGCATGATCTCCGGTATTGAAACTCCAGCGCGGGC
CATATCTCGCGCGGCTCCGACACGGGCACTGTGTCCAGACCAGGCCAGGTATCTCTGACCAGAGTC
ATCCTAAAATACACAAACAATTAGAATCAGTAGTTTAACACATTATACACTTAAAAATTTTATATT
TACCTTAGCGCCGTAAATCAATCGATGAGTTGCTTCAAAAATCCCTTCCAGGGCGCGAGTTGATAG
CTGGCTGGTGGCAGATGGCGCGGCAACACCATTTTTTCTGACCCGGCAAAACAGGTAGTTATTCGG
ATCATCAGCTACACCAGAGACGGAAATCCATCGCTCGACCAGTTTAGTGACTCCCAGGCTAAGTGC
CTTCTCTACACCTGCGGTGCTAACCAGCGTTTTCGTTCTGCCAATATGGATTAACATTCTCCCACC
GTCAGTACGTGAGATATCTTTAACCCTGATCCTGGCAATTTCGGCTATACGTAACAGGGTGTTATA
AGCAATCCCCAGAAATGCCAGATTACGTATATCCTGGCAGCGATCGCTATTTTCCATGAGTGAACG
GACTTGGTCGAAATCAGTGCGTTCGAACGCTAGAGCCTGTTTTGCACGTTCACCGGCATCAACGTT
TTCTTTTCGGATCCGCCGCATAACCAGTGAAACAGCATTGCTGTCACTTGGTCGTGGCAGCCCGGA
CCGACGATGAAGCATGTTTAGCTGGCCCAAATGTTGCTGGATAGTTTTTACTGCCAGACCGCGCGC
TTGAAGATATAGAAGATAATCGCGAACATCTTCAGGTTCTGCGGGAAACCATTTCCGGTTATTCAA
CTTGCACCATGCCGCCCACGACCGGCAAACGGACAGAAGCATTTTCCAGGTATGCTCAGAAAACGC
CTGGCGATCCCTGAACATGTCCATCAGGTTCTTGCGAACCTCATCACTCGTTGCATCGACCGGTAA
TGCAGGCAAATTTTGGTGTACGGTCAGTAAATTGGACATGGTGGCTACGTAATAACTTCGTATATG
GTTTCTTATACGAAGTTATGCGGCCGCTTTACGAGGGTAGGAAGTGGTACGGAAAGTTGGTATAAG
ACAAAAGTGTTGTGGAATTGCTCCAGGCGATCTGACGGTTCACTAAACGAGCTCTGCTTTTATAGG
CGCCCACCGTACACGCCTAAAGCTTATACGTTCTCTATCACTGATAGGGAGTAAACTGGATATACG
TTCTCTATCACTGATAGGGAGTAAACTGTAGATACGTTCTCTATCACTGATAGGGAGTAAACTGGT
CATACGTTCTCTATCACTGATAGGGAGTAAACTCCTTATACGTTCTCTATCACTGATAGGGAGTAA
AGTCTGCATACGTTCTCTATCACTGATAGGGAGTAAACTCTTCATACGTTCTCTATCACTGATAGG
GAGTAAACTCGCGGCCGCAGAGAAATGTTCTGGCACCTGCACTTGCACTGGGGACAGCCTATTTTG
CTAGTTTGTTTTGTTTCGTTTTGTTTTGATGGAGAGCGTATGTTAGTACTATCGATTCACACAAAA
AACCAACACACAGATGTAATGAAAATAAAGATATTTTATTGGATCTGCGATCGCTCCGGTGCCCGT
CAGTGGGCAGAGCGCACATCGCCCACAGTCCCCGAGAAGTTGGGGGGAGGGGTCGGCAATTGAACG
GGTGCCTAGAGAAGGTGGCGCGGGGTAAACTGGGAAAGTGATGTCGTGTACTGGCTCCGCCTTTTT
CCCGAGGGTGGGGGAGAACCGTATGTAAGTGCAGTAGTCGCCGTGAACGTTCTTTTTCGCAACGGG
TTTGCCGCCAGAACACAGCTGAAGCTTCGAGGGGCTCGCATCTCTCCTTCACGCGCCCGCCGCCCT
ACCTGAGGCCGCCATCCACGCCGGTTGAGTCGCGTTCTGCCGCCTCCCGCCTGTGGTGCCTCCTGA
ACTGCGTCCGCCGTCTAGGTAAGTTTAAAGCTCAGGTCGAGACCGGGCCTTTGTCCGGCGCTCCCT
TGGAGCCTACCTAGACTCAGCCGGCTCTCCACGCTTTGCCTGACCCTGCTTGCTCAACTCTACGTC
TTTGTTTCGTTTTCTGTTCTGCGCCGTTACAGATCCAAGCTGTGACCGGCGCCTACGCTAGCGGAT
CCGCCGCCACCATGTCTAGACTGGACAAGAGCAAAGTCATAAACTCTGCTCTGGAATTACTCAATG
GAGTCGGTATCGAAGGCCTGACGACAAGGAAACTCGCTCAAAAGCTGGGAGTTGAGCAGCCTACCC
TGTACTGGCACGTGAAGAACAAGCGGGCCCTGCTCGATGCCCTGCCAATCGAGATGCTGGACAGGC
ATCATACCCACTCCTGCCCCCTGGAAGGCGAGTCATGGCAAGACTTTCTGCGGAACAACGCCAAGT
CATACCGCTGTGCTCTTCTCTCACATCGCGACGGGGCTAAAGTGCATCTCGGCACCCGCCCAACAG
AGAAACAGTACGAAACCCTGGAAAATCAGCTCGCGTTCCTGTGTCAGCAAGGCTTCTCCCTGGAGA
ACGCACTGTACGCTCTGTCCGCCGTGGGCCACTTTACACTGGGCTGCGTATTGGAGGAACAGGAGC
ATCAAGTAGCAAAAGAGGAAAGAGAGACACCTACCACCGATTCTATGCCCCCACTTCTGAAACAAG
CAATTGAGCTGTTCGACCGGCAGGGAGCCGAACCTGCCTTCCTTTTCGGCCTGGAACTAATCATAT
GTGGCCTGGAGAAACAGCTAAAGTGCGAAAGCGGCGGGCCGACCGACGCCCTTGACGATTTTGACT
TAGACATGCTCCCAGCCGATGCCCTTGACGACTTTGACCTTGATATGCTGCCTGCTGACGCTCTTG
ACGATTTTGACCTTGACATGCTCCCCGGGGGATCCGGAGCTACTAACTTCAGCCTGCTGAAGCAGG
CTGGAGACGTGGAGGAGAACCCTGGACCTATGACAGAATATAAACCTACTGTCAGACTGGCAACTC
GAGACGACGTCCCTAGGGCCGTGAGAACATTGGCTGCCGCTTTCGCGGATTATCCCGCTACACGCC
ACACAGTTGATCCTGATAGACATATTGAACGGGTTACAGAATTGCAAGAACTTTTTTTGACCAGGG
TAGGATTGGACATCGGTAAAGTTTGGGTCGCCGACGACGGGGCTGCAGTGGCAGTGTGGACGACTC
CGGAGAGCGTTGAGGCCGGGGCTGTATTTGCAGAAATTGGTCCCCGAATGGCTGAGCTTAGTGGCT
CTCGTCTCGCGGCTCAGCAACAAATGGAAGGACTCCTCGCCCCTCACCGCCCTAAAGAACCAGCTT
GGTTCCTCGCTACTGTGGGCGTTAGCCCCGATCATCAGGGAAAGGGCCTTGGTTCCGCGGTGGTAT
TGCCCGGAGTAGAAGCCGCAGAACGAGCCGGAGTGCCAGCCTTTCTTGAAACGTCAGCGCCAAGGA
ATTTGCCCTTCTATGAACGGCTCGGATTTACAGTTACTGCTGACGTTGAAGTACCCGAGGGCCCAC
GGACGTGGTGCATGACGCGAAAACCCGGCGCTTGAGTTTAAACCGCTGATCAGCCTCGACTGTGCC
TTCTAGTTGCCAGCCATCTGTTGTTTGCCCCTCCCCCGTGCCTTCCTTGACCCTGGAAGGTGCCAC
TCCCACTGTCCTTTCCTAATAAAATGAGGAAATTGCATCGCATTGTCTGAGTAGGTGTCATTCTAT
TCTGGGGGGTGGGGTGGGGCAGGACAGCAAGGGGGAGGATTGGGAAGACAATAGCAGGCATGCTGG
GGATGCGGTGGGCTCTATGGCTTCTGAGGCGGAAAGAACCAGCTGGGGCTCGACTAGAGCTTGCGG
AACCCTTAGTTTAAACGGGCCCTTAATTAATCGATGTAGGATGTTGCCCCTCCTGACGCGGTAGGA
GAAGGGGAGGGTGCCCTGCATGTCTGCCGCTGCTCTTGCTCTTGCCGCTGCTGAGGAGGGGGGCGC
ATCTGCCGCAGCACCGGATGCATCTGGGAAAAGCAAAAAAGGGGCTCGTCCCTGTTTCCGGAGGAA
TTTGCAAGCGGGGTCTTGCATGACGGGGAGGCAAACCCCCGTTCGCCGCAGTCCGGCCGGCCCGAG
ACTCGAACCGGGGGTCCTGCGACTCAACCCTTGGAAAATAACCCTCCGGCTACAGGGAGCGAGCCA
CTTAATGCTTTCGCTTTCCAGCCTAACCGCTTACGCCGCGCGCGGCCAGTGGCCAAAAAAGCTAGC
GCAGCAGCCGCCGCGCCTGGAAGGAAGCCAAAAGGAGCGCTCCCCCGTTGTCTGACGTCGCACACC
TGGGTTCGACACGCGGGCGGTAACCGCATGGATCACGGCGGACGGCCGGATCCGGGGTTCGAACCC
CGGTCGTCCGCCATGATACCCTTGCGAATTTATCCACCAGACCACGGAAGAGTGCCCGCTTACAGG
CTCTCCTTTTGCACGGTCTAGAGCGTCAACGACTGCGCACGCCTCACCGGCCAGAGCGTCCCGACC
ATGGAGCACTTTTTGCCGCTGCGCAACATCTGGAACCGCGTCCGCGACTTTCCGCGCGCCTCCACC
ACCGCCGCCGGCATCACCTGGATGTCCAGGTACATCTACGGATTACGGGGCCCATTGGTATGGCTT
TTTCCCCGTATCCCCCCAGGTGTCTGCAGGCTCAAAGAGCAGCGAGAAGCGTTCAGAGGAAAGCGA
TCCCGTGCCACCTTCCCCGTGCCCGGGCTGTCCCCGCACGCTGCCGGCTCGGGGATGCGGGGGGAG
CGCCGGACCGGAGCGGAGCCCCGGGCGGCTCGCTGCTGCCCCCTAGCGGGGGAGGGACGTAATTAC
ATCCCTGGGGGCTTTGGGGGGGGGCTGTCCCTGATATCTATAACAAGAAAATATATATATAATAAG
TTATCACGTAAGTAGAACATGAAATAACAATATAATTATCGTATGAGTTAAATCTTAAAAGTCACG
TAAAAGATAATCATGCGTCATTTTGACTCACGCGGTCGTTATAGTTCAAAATCAGTGACACTTACC
GCATTGACAAGCACGCCTCACGGGAGCTCCAAGCGGCGACTGAGATGTCCTAAATGCACAGCGACG
GATTCGCGCTATTTAGAAAGAGAGAGCAATATTTCAAGAATGCATGCGTCAATTTTACGCAGACTA
TCTTTCTAGGGTTAATCTAGCTGCATCAGGATCATATCGTCGGGTCTTTTTTCCGGCTCAGTCATC
GCCCAAGCTGGCGCTATCTGGGCATCGGGGAGGAAGAAGCCCGTGCCTTTTCCCGCGAGGTTGAAG
CGGCATGGAAAGAGTTTGCCGAGGATGACTGCTGCTGCATTGACGTTGAGCGAAAACGCACGTTTA
CCATGATGATTCGGGAAGGTGTGGCCATGCACGCCTTTAACGGTGAACTGTTCGTTCAGGCCACCT
GGGATACCAGTTCGTCGCGGCTTTTCCGGACACAGTTCCGGATGGTCAGCCCGAAGCGCATCAGCA
ACCCGAACAATACCGGCGACAGCCGGAACTGCCGTGCCGGTGTGCAGATTAATGACAGCGGTGCGG
CGCTGGGATATTACGTCAGCGAGGACGGGTATCCTGGCTGGATGCCGCAGAAATGGACATGGATAC
CCCGTGAGTTACCCGGCGGGCGCGCTTGGCGTAATCATGGTCATAGCTGTTTCCTGTGTGAAATTG
TTATCCGCTCACAATTCCACACAACATACGAGCCGGAAGCATAAAGTGTAAAGCCTGGGGTGCCTA
ATGAGTGAGCTAACTCACATTAATTGCGTTGCGCTCACTGCCCGCTTTCCAGTCGGGAAACCTGTC
GTGCCAGCTGCATTAATGAATCGGCCAACGCGCGGGGAGAGGCGGTTTGCGTATTGGGCGCTCTTC
CGCTTCCTCGCTCACTGACTCGCTGCGCTCGGTCGTTCGGCTGCGGCGAGCGGTATCAGCTCACTC
AAAGGCGGTAATACGGTTATCCACAGAATCAGGGGATAACGCAGGAAAGAACATGTGAGCAAAAGG
CCAGCAAAAGGCCAGGAACCGTAAAAAGGCCGCGTTGCTGGCGTTTTTCCATAGGCTCCGCCCCCC
TGACGAGCATCACAAAAATCGACGCTCAAGTCAGAGGTGGCGAAACCCGACAGGACTATAAAGATA
CCAGGCGTTTCCCCCTGGAAGCTCCCTCGTGCGCTCTCCTGTTCCGACCCTGCCGCTTACCGGATA
CCTGTCCGCCTTTCTCCCTTCGGGAAGCGTGGCGCTTTCTCATAGCTCACGCTGTAGGTATCTCAG
TTCGGTGTAGGTCGTTCGCTCCAAGCTGGGCTGTGTGCACGAACCCCCCGTTCAGCCCGACCGCTG
CGCCTTATCCGGTAACTATCGTCTTGAGTCCAACCCGGTAAGACACGACTTATCGCCACTGGCAGC
AGCCACTGGTAACAGGATTAGCAGAGCGAGGTATGTAGGCGGTGCTACAGAGTTCTTGAAGTGGTG
GCCTAACTACGGCTACACTAGAAGGACAGTATTTGGTATCTGCGCTCTGCTGAAGCCAGTTACCTT
CGGAAAAAGAGTTGGTAGCTCTTGATCCGGCAAACAAACCACCGCTGGTAGCGGTGGTTTTTTTGT
TTGCAAGCAGCAGATTACGCGCAGAAAAAAAGGATCTCAAGAAGATCCTTTGATCTTTTCTACGGG
GTCTGACGCTCAGTGGAACGAAAACTCACGTTAAGGGATTTTGGTCATGAGATTATCAAAAAGGAT
CTTCACCTAGATCCTTTTAAATTAAAAATGAAGTTTTAAATCAATCTAAAGTATATATGAGTAAAC
TTGGTCTGACAGTTACCAATGCTTAATCAGTGAGGCACCTATCTCAGCGATCTGTCTATTTCGTTC
ATCCATAGTTGCCTGACTCCCCGTCGTGTAGATAACTACGATACGGGAGGGCTTACCATCTGGCCC
CAGTGCTGCAATGATACCGCGAGACCCACGCTCACCGGCTCCAGATTTATCAGCAATAAACCAGCC
AGCCGGAAGGGCCGAGCGCAGAAGTGGTCCTGCAACTTTATCCGCCTCCATCCAGTCTATTAATTG
TTGCCGGGAAGCTAGAGTAAGTAGTTCGCCAGTTAATAGTTTGCGCAACGTTGTTGCCATTGCTAC
AGGCATCGTGGTGTCACGCTCGTCGTTTGGTATGGCTTCATTCAGCTCCGGTTCCCAACGATCAAG
GCGAGTTACATGATCCCCCATGTTGTGCAAAAAAGCGGTTAGCTCCTTCGGTCCTCCGATCGTTGT
CAGAAGTAAGTTGGCCGCAGTGTTATCACTCATGGTTATGGCAGCACTGCATAATTCTCTTACTGT
CATGCCATCCGTAAGATGCTTTTCTGTGACTGGTGAGTACTCAACCAAGTCATTCTGAGAATAGTG
TATGCGGCGACCGAGTTGCTCTTGCCCGGCGTCAATACGGGATAATACCGCGCCACATAGCAGAAC
TTTAAAAGTGCTCATCATTGGAAAACGTTCTTCGGGGCGAAAACTCTCAAGGATCTTACCGCTGTT
GAGATCCAGTTCGATGTAACCCACTCGTGCACCCAACTGATCTTCAGCATCTTTTACTTTCACCAG
CGTTTCTGGGTGAGCAAAAACAGGAAGGCAAAATGCCGCAAAAAAGGGAATAAGGGCGACACGGAA
ATGTTGAATACTCAT
SEQ ID NO: 11 (Helper construct, V1)
GCCTCCACGGCCACTAGTCCATAGAGCCCACCGCATCCCCAGCATGCCTGCTATTGTCTTCCCAAT
CCTCCCCCTTGCTGTCCTGCCCCACCCCACCCCCTAGAATAGAATGACACCTACTCAGACAATGCG
ATGCAATTTCCTCATTTTATTAGGAAAGGACAGTGGGAGTGGCACCTTCCAGGGTCAAGGAAGGCA
CGGGGGAGGGGCAAACAACAGATGGCTGGCAACTAGAAGGCACAGCTACATGGGGGTAGAGTCATA
ATCGTGCATCAGGATAGGGCGGTGGTGCTGCAGCAGCGCGCGAATAAACTGCTGCCGCCGCCGCTC
CGTCCTGCAGGAATACAACATGGCAGTGGTCTCCTCAGCGATGATTCGCACCGCCCGCAGCATGAG
ACGCCTTGTCCTCCGGGCACAGCAGCGCACCCTGATCTCACTTAAATCAGCACAGTAACTGCAGCA
CAGCACCACAATATTGTTCAAAATCCCACAGTGCAAGGCGCTGTATCCAAAGCTCATGGCGGGGAC
CACAGAACCCACGTGGCCATCATACCACAAGCGCAGGTAGATTAAGTGGCGACCCCTCATAAACAC
GCTGGACATAAACATTACCTCTTTTGGCATGTTGTAATTCACCACCTCCCGGTACCATATAAACCT
CTGATTAAACATGGCGCCATCCACCACCATCCTAAACCAGCTGGCCAAAACCTGCCCGCCGGCTAT
GCACTGCAGGGAACCGGGACTGGAACAATGACAGTGGAGAGCCCAGGACTCGTAACCATGGATCAT
CATGCTCGTCATGATATCAATGTTGGCACAACACAGGCACACGTGCATACACTTCCTCAGGATTAC
AAGCTCCTCCCGCGTCAGAACCATATCCCAGGGAACAACCCATTCCTGAATCAGCGTAAATCCCAC
ACTGCAGGGAAGACCTCGCACGTAACTCACGTTGTGCATTGTCAAAGTGTTACATTCGGGCAGCAG
CGGATGATCCTCCAGTATGGTAGCGCGGGTCTCTGTCTCAAAAGGAGGTAGGCGATCCCTACTGTA
CGGAGTGCGCCGAGACAACCGAGATCGTGTTGGTCGTAGTGTCATGCCAAATGGAACGCCGGACGT
AGTCATGGTTGTGGCCATATTATCATCGTGTTTTTCAAAGGAAAACCACGTCCCCGTGGTTCGGGG
GGCCTAGACGTTTTTTTAACCTCGACTAAACACATGTAAAGCATGTGCACCGAGGCCCCAGATCAG
ATCCCATACAATGGGGTACCTTCTGGGCATCCTTCAGCCCCTTGTTGAATACGCTTGAGGAGAGCC
ATTTGACTCTTTCCACAACTATCCAACTCACAACGTGGCACTGGGGTTGTGCCGCCTTTGCAGGTG
TATCTTATACACGTGGCTTTTGGCCGCAGAGGCACCTGTCGCCAGGTGGGGGGTTCCGCTGCCTGC
AAAGGGTCGCTACAGACGTTGTTTGTCTTCAAGAAGCTTCCAGAGGAACTGCTTCCTTCACGACAT
TCAACAGACCTTGCATTCCTTTGGCGAGAGGGGAAAGACCCCTAGGAATGCTCGTCAAGAAGACAG
GGCCAGGTTTCCGGGCCCTCACATTGCCAAAAGACGGCAATATGGTGGAAAATAACATATAGACAA
ACGCACACCGGCCTTATTCCAAGCGGCTTCGGCCAGTAACGTTAGGGGGGGGGGAGGGAGAGGGGC
TTAAAAATCAAAGGGGTTCTGCCGCGCATCACTATGCGCCACTGGCAGGGACACGTTGCGATACTG
GTGTTTAGTGCTCCACTTAAACTCAGGCACAACCATCCGCGGCAGCTCGGTGAAGTTTTCACTCCA
CAGGCTGCGCACCATCACCAACGCGTTTAGCAGGTCGGGCGCCGATATCTTGAAGTCGCAGTTGGG
GCCTCCGCCCTGCGCGCGCGAGTTGCGATACACAGGGTTGCAGCACTGGAACACTATCAGCGCCGG
GTGGTGCACGCTGGCCAGCACGCTCTTGTCGGAGATCAGATCCGCGTCCAGGTCCTCCGCGTTGCT
CAGGGCGAACGGAGTCAACTTTGGTAGCTGCCTTCCCAAAAAGGGTGCATGCCCAGGCTTTGAGTT
GCACTCGCACCGTAGTGGCATCAGAAGGTGACCGTGCCCGGTCTGGGCGTTAGGATACAGCGCCTG
CATGAAAGCCTTGATCTGCTTAAAAGCCACCTGAGCCTTTGCGCCTTCAGAGAAGAACATGCCGCA
AGACTTGCCGGAAAACTGATTGGCCGGACAGGCCGCGTCATGCACGCAGCACCTTGCGTCGGTGTT
GGAGATCTGCACCACATTTCGGCCCCACCGGTTCTTCACGATCTTGGCCTTGCTAGACTGCTCCTT
CAGCGCGCGCTGCCCGTTTTCGCTCGTCACATCCATTTCAATCACGTGCTCCTTATTTATCATAAT
GCTCCCGTGTAGACACTTAAGCTCGCCTTCGATCTCAGCGCAGCGGTGCAGCCACAACGCGCAGCC
CGTGGGCTCGTGGTGCTTGTAGGTTACCTCTGCAAACGACTGCAGGTACGCCTGCAGGAATCGCCC
CATCATCGTCACAAAGGTCTTGTTGCTGGTGAAGGTCAGCTGCAACCCGCGGTGCTCCTCGTTTAG
CCAGGTCTTGCATACGGCCGCCAGAGCTTCCACTTGGTCAGGCAGTAGCTTGAAGTTTGCCTTTAG
ATCGTTATCCACGTGGTACTTGTCCATCAACGCGCGCGCAGCCTCCATGCCCTTCTCCCACGCAGA
CACGATCGGCAGGCTCAGCGGGTTTATCACCGTGCTTTCACTTTCCGCTTCACTGGACTCTTCCTT
TTCCTCTTGCGTCCGCATACCCCGCGCCACTGGGTCGTCTTCATTCAGCCGCCGCACCGTGCGCTT
ACCTCCCTTGCCGTGCTTGATTAGCACCGGTGGGTTGCTGAAACCCACCATTTGTAGCGCCACATC
TTCTCTTTCTTCCTCGCTGTCCACGATCACCTCTGGGGATGGCGGGCGCTCGGGCTTGGGAGAGGG
GCGCTTCTTTTTCTTTTTGGACGCAATGGCCAAATCCGCCGTCGAGGTCGATGGCCGCGGGCTGGG
TGTGCGCGGCACCAGCGCATCTTGTGACGAGTCTTCTTCGTCCTCGGACTCGAGACGCCGCCTCAG
CCGCTTTTTTGGGGGCGCGCGCTTGTCGTCATCGTCTTTGTAGTCGGGAGGCGGCGGCGACGGCGA
CGGGGACGACACGTCCTCCATGGTTGGTGGACGTCGCGCCGCACCGCGTCCGCGCTCGGGGGTGGT
TTCGCGCTGCTCCTCTTCCCGACTGGCCATGGTGGCCGAGGATAACTTCGTATATGGTTTCTTATA
CGAAGTTATGATCCAGACATGATAAGATACATTGATGAGTTTGGACAAACCACAACTAGAATGCAG
TGAAAAAAATGCTTTATTTGTGAAATTTGTGATGCTATTGCTTTATTTGTAACCATTATAAGCTGC
AATAAACAAGTTAACAACAACAATTGCATTCATTTTATGTTTCAGGTTCAGGGGGAGGTGTGGGAG
GTTTTTTAAAGCAAGTAAAACCTCTACAAATGTGGTATGGCTGATTATGATCCTCTAGAGTCGCAG
ATCTGCTACGTATCAAGCTGTGGCAGGGAAACCCTCTGCCTCCCCCGTGATGTAATACTTTTGCAA
GGAATGCGATGAAGTAGAGCCCGCAGTGGCCAAGTGGCTTTGGTCCGTCTCCTCCACGGATGCCCC
TCCACGGCTAGTGGGCGCATGTAGGCGGTGGGCGTCCGCCGCCTCCAGCAGCAGGTCATAGAGGGG
CACCACGTTCTTGCACTTCATGCTGTACAGATGCTCCATGCCTTTGTTACTCATGTGTCGGATGTG
GGAGAGGATGAGGAGGAGCTGGGCCAGCCGCTGGTGCTGCTGCTGCAGGGTCAGGCCTGCCTTGGC
CATCAGGTGGATCAAAGTGTCTGTGATCTTGTCCAGGACTCGGTGGATATGGTCCTTCTCTTCCAG
AGACTTCAGGGTGCTGGACAGAAATGTGTACACTCCAGAATTAAGCAAAATAATAGATTTGAGGCA
CACAAACTCCTCTCCCTGCAGATTCATCATGCGGAACCGAGATGATGTAGCCAGCAGCATGTCGAA
GATCTCCACCATGCCCTCTACACATTTTCCCTGGTTCCTGTCCAAGAGCAAGTTAGGAGCAAACAG
TAGCTTCACTGGGTGCTCCATGGAGCGCCAGACGAGACCAATCATCAGGATCTCTAGCCAGGCACA
TTCTAGAAGGTGGACCTGATCATGGAGGGTCAAATCCACAAAGCCTGGCACCCTCTTCGCCCAGTT
GATCATGTGAACCAGCTCCCTGTCTGCCAGGTTGGTCAGTAAGCCCATCATCGAAGCTTCACTGAA
GGGTCTGGTAGGATCATACTCGGAATAGAGTATGGGGGGCTCAGCATCCAACAAGGCACTGACCAT
CTGGTCGGCCGTCAGGGACAAGGCCAGGCTGTTCTTCTTAGAGCGTTTGATCATGAGCGGGCTTGG
CCAAAGGTTGGCAGCTCTCATGTCTCCAGCAGATGGCTCGAGATCGCCATCTTCCAGCAGGCGCAC
CATTGCCCCTGTTTCACTATCCAGGTTACGGATATAGTTCATGACAATATTTACATTGGTCCAGCC
ACCAGCTTGCATGATCTCCGGTATTGAAACTCCAGCGCGGGCCATATCTCGCGCGGCTCCGACACG
GGCACTGTGTCCAGACCAGGCCAGGTATCTCTGACCAGAGTCATCCTAAAATACACAAACAATTAG
AATCAGTAGTTTAACACATTATACACTTAAAAATTTTATATTTACCTTAGCGCCGTAAATCAATCG
ATGAGTTGCTTCAAAAATCCCTTCCAGGGCGCGAGTTGATAGCTGGCTGGTGGCAGATGGCGCGGC
AACACCATTTTTTCTGACCCGGCAAAACAGGTAGTTATTCGGATCATCAGCTACACCAGAGACGGA
AATCCATCGCTCGACCAGTTTAGTGACTCCCAGGCTAAGTGCCTTCTCTACACCTGCGGTGCTAAC
CAGCGTTTTCGTTCTGCCAATATGGATTAACATTCTCCCACCGTCAGTACGTGAGATATCTTTAAC
CCTGATCCTGGCAATTTCGGCTATACGTAACAGGGTGTTATAAGCAATCCCCAGAAATGCCAGATT
ACGTATATCCTGGCAGCGATCGCTATTTTCCATGAGTGAACGGACTTGGTCGAAATCAGTGCGTTC
GAACGCTAGAGCCTGTTTTGCACGTTCACCGGCATCAACGTTTTCTTTTCGGATCCGCCGCATAAC
CAGTGAAACAGCATTGCTGTCACTTGGTCGTGGCAGCCCGGACCGACGATGAAGCATGTTTAGCTG
GCCCAAATGTTGCTGGATAGTTTTTACTGCCAGACCGCGCGCTTGAAGATATAGAAGATAATCGCG
AACATCTTCAGGTTCTGCGGGAAACCATTTCCGGTTATTCAACTTGCACCATGCCGCCCACGACCG
GCAAACGGACAGAAGCATTTTCCAGGTATGCTCAGAAAACGCCTGGCGATCCCTGAACATGTCCAT
CAGGTTCTTGCGAACCTCATCACTCGTTGCATCGACCGGTAATGCAGGCAAATTTTGGTGTACGGT
CAGTAAATTGGACATGGTGGCTACGTAATAACTTCGTATATGGTTTCTTATACGAAGTTATGCGGC
CGCTTTACGAGGGTAGGAAGTGGTACGGAAAGTTGGTATAAGACAAAAGTGTTGTGGAATTGCTCC
AGGCGATCTGACGGTTCACTAAACGAGCTCTGCTTTTATAGGCGCCCACCGTACACGCCTAAAGCT
TATACGTTCTCTATCACTGATAGGGAGTAAACTGGATATACGTTCTCTATCACTGATAGGGAGTAA
ACTGTAGATACGTTCTCTATCACTGATAGGGAGTAAACTGGTCATACGTTCTCTATCACTGATAGG
GAGTAAACTCCTTATACGTTCTCTATCACTGATAGGGAGTAAAGTCTGCATACGTTCTCTATCACT
GATAGGGAGTAAACTCTTCATACGTTCTCTATCACTGATAGGGAGTAAACTCGCGGCCGCAGAGAA
ATGTTCTGGCACCTGCACTTGCACTGGGGACAGCCTATTTTGCTAGTTTGTTTTGTTTCGTTTTGT
TTTGATGGAGAGCGTATGTTAGTACTATCGATTCACACAAAAAACCAACACACAGATGTAATGAAA
ATAAAGATATTTTATTGGATCTGCGATCGCTCCGGTGCCCGTCAGTGGGCAGAGCGCACATCGCCC
ACAGTCCCCGAGAAGTTGGGGGGAGGGGTCGGCAATTGAACGGGTGCCTAGAGAAGGTGGCGCGGG
GTAAACTGGGAAAGTGATGTCGTGTACTGGCTCCGCCTTTTTCCCGAGGGTGGGGGAGAACCGTAT
GTAAGTGCAGTAGTCGCCGTGAACGTTCTTTTTCGCAACGGGTTTGCCGCCAGAACACAGCTGAAG
CTTCGAGGGGCTCGCATCTCTCCTTCACGCGCCCGCCGCCCTACCTGAGGCCGCCATCCACGCCGG
TTGAGTCGCGTTCTGCCGCCTCCCGCCTGTGGTGCCTCCTGAACTGCGTCCGCCGTCTAGGTAAGT
TTAAAGCTCAGGTCGAGACCGGGCCTTTGTCCGGCGCTCCCTTGGAGCCTACCTAGACTCAGCCGG
CTCTCCACGCTTTGCCTGACCCTGCTTGCTCAACTCTACGTCTTTGTTTCGTTTTCTGTTCTGCGC
CGTTACAGATCCAAGCTGTGACCGGCGCCTACGCTAGCGGATCCGCCGCCACCATGTCTAGACTGG
ACAAGAGCAAAGTCATAAACTCTGCTCTGGAATTACTCAATGGAGTCGGTATCGAAGGCCTGACGA
CAAGGAAACTCGCTCAAAAGCTGGGAGTTGAGCAGCCTACCCTGTACTGGCACGTGAAGAACAAGC
GGGCCCTGCTCGATGCCCTGCCAATCGAGATGCTGGACAGGCATCATACCCACTCCTGCCCCCTGG
AAGGCGAGTCATGGCAAGACTTTCTGCGGAACAACGCCAAGTCATACCGCTGTGCTCTTCTCTCAC
ATCGCGACGGGGCTAAAGTGCATCTCGGCACCCGCCCAACAGAGAAACAGTACGAAACCCTGGAAA
ATCAGCTCGCGTTCCTGTGTCAGCAAGGCTTCTCCCTGGAGAACGCACTGTACGCTCTGTCCGCCG
TGGGCCACTTTACACTGGGCTGCGTATTGGAGGAACAGGAGCATCAAGTAGCAAAAGAGGAAAGAG
AGACACCTACCACCGATTCTATGCCCCCACTTCTGAAACAAGCAATTGAGCTGTTCGACCGGCAGG
GAGCCGAACCTGCCTTCCTTTTCGGCCTGGAACTAATCATATGTGGCCTGGAGAAACAGCTAAAGT
GCGAAAGCGGCGGGCCGACCGACGCCCTTGACGATTTTGACTTAGACATGCTCCCAGCCGATGCCC
TTGACGACTTTGACCTTGATATGCTGCCTGCTGACGCTCTTGACGATTTTGACCTTGACATGCTCC
CCGGGTGAACCGGTCGCTGATCAGCCTCGACTGTGCCTTCTAGTTGCCAGCCATCTGTTGTTTGCC
CCTCCCCCGTGCCTTCCTTGACCCTGGAAGGTGCCACTCCCACTGTCCTTTCCTAATAAAATGAGG
AAATTGCATCGCATTGTCTGAGTAGGTGTCATTCTATTCTGGGGGGTGGGGTGGGGCAGGACAGCA
AGGGGGAGGATTGGGAAGACAATAGCAGGCATGCTGGGGATGCGGTGGGCTCTATGGCTTCTGAGG
CGGAAAGAACCAGCTGGGGCTCGACTAGAGCTTGCGGAACCCTTAGAGGGCCTATTTCCCATGATT
CCTTCATATTTGCATATACGATACAAGGCTGTTAGAGAGATAATTAGAATTAATTTGACTGTAAAC
ACAAAGATATTAGTACAAAATAATAACTTCGTATAATGTATGCTATACGAAGTTATCAGACATGAT
AAGATACATTGATGAGTTTGGACAAACCACAACTAGAATGCAGTGAAAAAAATGCTTTATTTGTGA
AATTTGTGATGCTATTGCTTTATTTGTAACCATTATAAGCTGCAATAAACAAGGTACCTCAAGCGC
CGGGTTTTCGCGTCATGCACCACGTCCGTGGGCCCTCGGGTACTTCAACGTCAGCAGTAACTGTAA
ATCCGAGCCGTTCATAGAAGGGCAAATTCCTTGGCGCTGACGTTTCAAGAAAGGCTGGCACTCCGG
CTCGTTCTGCGGCTTCTACTCCGGGCAATACCACCGCGGAACCAAGGCCCTTTCCCTGATGATCGG
GGCTAACGCCCACAGTAGCGAGGAACCAAGCTGGTTCTTTAGGGCGGTGAGGGGCGAGGAGTCCTT
CCATTTGTTGCTGAGCCGCGAGACGAGAGCCACTAAGCTCAGCCATTCGGGGACCAATTTCTGCAA
ATACAGCCCCGGCCTCAACGCTCTCCGGAGTCGTCCACACTGCCACTGCAGCCCCGTCGTCGGCGA
CCCAAACTTTACCGATGTCCAATCCTACCCTGGTCAAAAAAAGTTCTTGCAATTCTGTAACCCGTT
CAATATGTCTATCAGGATCAACTGTGTGGCGTGTAGCGGGATAATCCGCGAAAGCGGCAGCCAATG
TTCTCACGGCCCTAGGGACGTCGTCTCGAGTTGCCAGTCTGACAGTAGGTTTATATTCTGTCATAG
GTCCAGGGTTCTCCTCCACGTCTCCAGCCTGCTTCAGCAGGCTGAAGTTAGTAGCTCCGGATCCTT
TACCTCCATCACCAGCGCCACCAGTAGAGTATCTGGCCACAGCCACCTCGTGCTGCTCGACGTAGG
TCTCATCGTCGGCCTCCTTGATTCTTTCCAGTCTGTGGTCCACGTTGTAGACGCCGGGCATCTTGA
GGTTCGTAGCGGGTTTCTTGGATCTGTATGTGGTCTCAAGGTTGCAGATCAGGTGGCCCCCGCCCA
CGAGCTTCAGGGCCATGTCACATGCGCCTTCCAGGCCGCCGTCAGCGGGGTACATCGTCTCGGTGG
AGGCCTCCCAGCCGAGTGTTTTCTTCTGCATCACAGGGCCGTTGGCTGGGAAGTTCACCCCTCTAA
CCTTGACGTTGTAGATGAGGCAGCCGTCCTGGAGGCTGGTGTCCTGGGTAGCGGTCAGCACGCCCC
CGTCTTCGTATGTGGTGACTCTCTCCCATGTGAAGCCCTCAGGGAAGGACTGCTTAAAGAAGTCGG
GGATGCCCGGAGGGTGCTTGATGAAGGTTCTGCTGCCGTACATGAAGCTGGTAGCCAGGATGTCGA
AGGCGAAGGGGAGAGGGCCGCCCTCGACGACCTTGATTCTCATGGTCTGGGTGCCCTCGTAGGGCT
TGCCTTCGCCCTCGGATGTGCACTTGAAGTGGTGGTTGTTCACGGTGCCCTCCATGTACAGCTTCA
TGGGCATGTTCTCCTTAATCAGCTCGCTCACGGTGGCGGCGAATTCCGAAAGGCCCGGAGATGAGG
AAGAGGAGAACAGCGCGGCAGACGTGCGCTTTTGAAGCGTGCAGAATGCCGGGCCTCCGGAGGACC
TTCGGGCGCCCGCCCCGCCCCTGAGCCCGCCCCTGAGCCCGCCCCCGGACCCACCCCTTCCCAGCC
TCTGAGCCCAGAAAGCGAAGGAGCAAAGCTGCTATTGGCCGCTGCCCCAAAGGCCTACCCGCTTCC
ATTGCTCAGCGGTGCTGTCCATCTGCACGAGACTAGTGAGACGTGCTACTTCCATTTGTCACGTCC
TGCACGACGCGAGCTGCGGGGGGGGGGGGAACTTCCTGACTAGGGGAGGAGTAGAAGGTGGCGCGA
AGGGGCCACCAAAGAACGGAGCCGGTTGGCGCCTACCGGTGGATGTGGAATGTGTGCGAGGCCAGA
GGCCACTTGTGTAGCGCCAAGTGCCCAGCGGGGCTGCTAAAGCGCATGCTCCAGACTGCCTTGGGA
AAAGCGCTCCCCTACCCATAACTTCGTATAATGTATGCTATACGAAGTTATTTTGCAGTTTTAAAA
TTATGTTTTAAAATGGACTATCATATGCTTACCGTAACTTGAAAGTATTTCGATTTCTTGGCTTTA
TATATCTTGTGGAAAGGACGAAACACCGGGCACTCTTCCGTGATCTGGTGGATAAATTCGCAAGGG
TATCATGGCGGACGACCGGGATTCGAACCCCGGATCCGGCCGTCCGCCGTGATCCATGCGGTTACC
GCCCGCGTGTCGAACCCAGGTGTGCGACGTCAGACAACGGGGGAGCGCTCC
SEQ ID NO: 12 (Helper construct, v2)
GCCTCCACGGCCACTAGTCCATAGAGCCCACCGCATCCCCAGCATGCCTGCTATTGTCTTCCCAAT
CCTCCCCCTTGCTGTCCTGCCCCACCCCACCCCCTAGAATAGAATGACACCTACTCAGACAATGCG
ATGCAATTTCCTCATTTTATTAGGAAAGGACAGTGGGAGTGGCACCTTCCAGGGTCAAGGAAGGCA
CGGGGGAGGGGCAAACAACAGATGGCTGGCAACTAGAAGGCACAGCTACATGGGGGTAGAGTCATA
ATCGTGCATCAGGATAGGGCGGTGGTGCTGCAGCAGCGCGCGAATAAACTGCTGCCGCCGCCGCTC
CGTCCTGCAGGAATACAACATGGCAGTGGTCTCCTCAGCGATGATTCGCACCGCCCGCAGCATGAG
ACGCCTTGTCCTCCGGGCACAGCAGCGCACCCTGATCTCACTTAAATCAGCACAGTAACTGCAGCA
CAGCACCACAATATTGTTCAAAATCCCACAGTGCAAGGCGCTGTATCCAAAGCTCATGGCGGGGAC
CACAGAACCCACGTGGCCATCATACCACAAGCGCAGGTAGATTAAGTGGCGACCCCTCATAAACAC
GCTGGACATAAACATTACCTCTTTTGGCATGTTGTAATTCACCACCTCCCGGTACCATATAAACCT
CTGATTAAACATGGCGCCATCCACCACCATCCTAAACCAGCTGGCCAAAACCTGCCCGCCGGCTAT
GCACTGCAGGGAACCGGGACTGGAACAATGACAGTGGAGAGCCCAGGACTCGTAACCATGGATCAT
CATGCTCGTCATGATATCAATGTTGGCACAACACAGGCACACGTGCATACACTTCCTCAGGATTAC
AAGCTCCTCCCGCGTCAGAACCATATCCCAGGGAACAACCCATTCCTGAATCAGCGTAAATCCCAC
ACTGCAGGGAAGACCTCGCACGTAACTCACGTTGTGCATTGTCAAAGTGTTACATTCGGGCAGCAG
CGGATGATCCTCCAGTATGGTAGCGCGGGTCTCTGTCTCAAAAGGAGGTAGGCGATCCCTACTGTA
CGGAGTGCGCCGAGACAACCGAGATCGTGTTGGTCGTAGTGTCATGCCAAATGGAACGCCGGACGT
AGTCATGGTTGTGGCCATATTATCATCGTGTTTTTCAAAGGAAAACCACGTCCCCGTGGTTCGGGG
GGCCTAGACGTTTTTTTAACCTCGACTAAACACATGTAAAGCATGTGCACCGAGGCCCCAGATCAG
ATCCCATACAATGGGGTACCTTCTGGGCATCCTTCAGCCCCTTGTTGAATACGCTTGAGGAGAGCC
ATTTGACTCTTTCCACAACTATCCAACTCACAACGTGGCACTGGGGTTGTGCCGCCTTTGCAGGTG
TATCTTATACACGTGGCTTTTGGCCGCAGAGGCACCTGTCGCCAGGTGGGGGGTTCCGCTGCCTGC
AAAGGGTCGCTACAGACGTTGTTTGTCTTCAAGAAGCTTCCAGAGGAACTGCTTCCTTCACGACAT
TCAACAGACCTTGCATTCCTTTGGCGAGAGGGGAAAGACCCCTAGGAATGCTCGTCAAGAAGACAG
GGCCAGGTTTCCGGGCCCTCACATTGCCAAAAGACGGCAATATGGTGGAAAATAACATATAGACAA
ACGCACACCGGCCTTATTCCAAGCGGCTTCGGCCAGTAACGTTAGGGGGGGGGGAGGGAGAGGGGC
TTAAAAATCAAAGGGGTTCTGCCGCGCATCACTATGCGCCACTGGCAGGGACACGTTGCGATACTG
GTGTTTAGTGCTCCACTTAAACTCAGGCACAACCATCCGCGGCAGCTCGGTGAAGTTTTCACTCCA
CAGGCTGCGCACCATCACCAACGCGTTTAGCAGGTCGGGCGCCGATATCTTGAAGTCGCAGTTGGG
GCCTCCGCCCTGCGCGCGCGAGTTGCGATACACAGGGTTGCAGCACTGGAACACTATCAGCGCCGG
GTGGTGCACGCTGGCCAGCACGCTCTTGTCGGAGATCAGATCCGCGTCCAGGTCCTCCGCGTTGCT
CAGGGCGAACGGAGTCAACTTTGGTAGCTGCCTTCCCAAAAAGGGTGCATGCCCAGGCTTTGAGTT
GCACTCGCACCGTAGTGGCATCAGAAGGTGACCGTGCCCGGTCTGGGCGTTAGGATACAGCGCCTG
CATGAAAGCCTTGATCTGCTTAAAAGCCACCTGAGCCTTTGCGCCTTCAGAGAAGAACATGCCGCA
AGACTTGCCGGAAAACTGATTGGCCGGACAGGCCGCGTCATGCACGCAGCACCTTGCGTCGGTGTT
GGAGATCTGCACCACATTTCGGCCCCACCGGTTCTTCACGATCTTGGCCTTGCTAGACTGCTCCTT
CAGCGCGCGCTGCCCGTTTTCGCTCGTCACATCCATTTCAATCACGTGCTCCTTATTTATCATAAT
GCTCCCGTGTAGACACTTAAGCTCGCCTTCGATCTCAGCGCAGCGGTGCAGCCACAACGCGCAGCC
CGTGGGCTCGTGGTGCTTGTAGGTTACCTCTGCAAACGACTGCAGGTACGCCTGCAGGAATCGCCC
CATCATCGTCACAAAGGTCTTGTTGCTGGTGAAGGTCAGCTGCAACCCGCGGTGCTCCTCGTTTAG
CCAGGTCTTGCATACGGCCGCCAGAGCTTCCACTTGGTCAGGCAGTAGCTTGAAGTTTGCCTTTAG
ATCGTTATCCACGTGGTACTTGTCCATCAACGCGCGCGCAGCCTCCATGCCCTTCTCCCACGCAGA
CACGATCGGCAGGCTCAGCGGGTTTATCACCGTGCTTTCACTTTCCGCTTCACTGGACTCTTCCTT
TTCCTCTTGCGTCCGCATACCCCGCGCCACTGGGTCGTCTTCATTCAGCCGCCGCACCGTGCGCTT
ACCTCCCTTGCCGTGCTTGATTAGCACCGGTGGGTTGCTGAAACCCACCATTTGTAGCGCCACATC
TTCTCTTTCTTCCTCGCTGTCCACGATCACCTCTGGGGATGGCGGGCGCTCGGGCTTGGGAGAGGG
GCGCTTCTTTTTCTTTTTGGACGCAATGGCCAAATCCGCCGTCGAGGTCGATGGCCGCGGGCTGGG
TGTGCGCGGCACCAGCGCATCTTGTGACGAGTCTTCTTCGTCCTCGGACTCGAGACGCCGCCTCAG
CCGCTTTTTTGGGGGCGCGCGCTTGTCGTCATCGTCTTTGTAGTCGGGAGGCGGCGGCGACGGCGA
CGGGGACGACACGTCCTCCATGGTTGGTGGACGTCGCGCCGCACCGCGTCCGCGCTCGGGGGTGGT
TTCGCGCTGCTCCTCTTCCCGACTGGCCATGGTGGCCGAGGATAACTTCGTATATGGTTTCTTATA
CGAAGTTATGATCCAGACATGATAAGATACATTGATGAGTTTGGACAAACCACAACTAGAATGCAG
TGAAAAAAATGCTTTATTTGTGAAATTTGTGATGCTATTGCTTTATTTGTAACCATTATAAGCTGC
AATAAACAAGTTAACAACAACAATTGCATTCATTTTATGTTTCAGGTTCAGGGGGAGGTGTGGGAG
GTTTTTTAAAGCAAGTAAAACCTCTACAAATGTGGTATGGCTGATTATGATCCTCTAGAGTCGCAG
ATCTGCTACGTATCAAGCTGTGGCAGGGAAACCCTCTGCCTCCCCCGTGATGTAATACTTTTGCAA
GGAATGCGATGAAGTAGAGCCCGCAGTGGCCAAGTGGCTTTGGTCCGTCTCCTCCACGGATGCCCC
TCCACGGCTAGTGGGCGCATGTAGGCGGTGGGCGTCCGCCGCCTCCAGCAGCAGGTCATAGAGGGG
CACCACGTTCTTGCACTTCATGCTGTACAGATGCTCCATGCCTTTGTTACTCATGTGTCGGATGTG
GGAGAGGATGAGGAGGAGCTGGGCCAGCCGCTGGTGCTGCTGCTGCAGGGTCAGGCCTGCCTTGGC
CATCAGGTGGATCAAAGTGTCTGTGATCTTGTCCAGGACTCGGTGGATATGGTCCTTCTCTTCCAG
AGACTTCAGGGTGCTGGACAGAAATGTGTACACTCCAGAATTAAGCAAAATAATAGATTTGAGGCA
CACAAACTCCTCTCCCTGCAGATTCATCATGCGGAACCGAGATGATGTAGCCAGCAGCATGTCGAA
GATCTCCACCATGCCCTCTACACATTTTCCCTGGTTCCTGTCCAAGAGCAAGTTAGGAGCAAACAG
TAGCTTCACTGGGTGCTCCATGGAGCGCCAGACGAGACCAATCATCAGGATCTCTAGCCAGGCACA
TTCTAGAAGGTGGACCTGATCATGGAGGGTCAAATCCACAAAGCCTGGCACCCTCTTCGCCCAGTT
GATCATGTGAACCAGCTCCCTGTCTGCCAGGTTGGTCAGTAAGCCCATCATCGAAGCTTCACTGAA
GGGTCTGGTAGGATCATACTCGGAATAGAGTATGGGGGGCTCAGCATCCAACAAGGCACTGACCAT
CTGGTCGGCCGTCAGGGACAAGGCCAGGCTGTTCTTCTTAGAGCGTTTGATCATGAGCGGGCTTGG
CCAAAGGTTGGCAGCTCTCATGTCTCCAGCAGATGGCTCGAGATCGCCATCTTCCAGCAGGCGCAC
CATTGCCCCTGTTTCACTATCCAGGTTACGGATATAGTTCATGACAATATTTACATTGGTCCAGCC
ACCAGCTTGCATGATCTCCGGTATTGAAACTCCAGCGCGGGCCATATCTCGCGCGGCTCCGACACG
GGCACTGTGTCCAGACCAGGCCAGGTATCTCTGACCAGAGTCATCCTAAAATACACAAACAATTAG
AATCAGTAGTTTAACACATTATACACTTAAAAATTTTATATTTACCTTAGCGCCGTAAATCAATCG
ATGAGTTGCTTCAAAAATCCCTTCCAGGGCGCGAGTTGATAGCTGGCTGGTGGCAGATGGCGCGGC
AACACCATTTTTTCTGACCCGGCAAAACAGGTAGTTATTCGGATCATCAGCTACACCAGAGACGGA
AATCCATCGCTCGACCAGTTTAGTGACTCCCAGGCTAAGTGCCTTCTCTACACCTGCGGTGCTAAC
CAGCGTTTTCGTTCTGCCAATATGGATTAACATTCTCCCACCGTCAGTACGTGAGATATCTTTAAC
CCTGATCCTGGCAATTTCGGCTATACGTAACAGGGTGTTATAAGCAATCCCCAGAAATGCCAGATT
ACGTATATCCTGGCAGCGATCGCTATTTTCCATGAGTGAACGGACTTGGTCGAAATCAGTGCGTTC
GAACGCTAGAGCCTGTTTTGCACGTTCACCGGCATCAACGTTTTCTTTTCGGATCCGCCGCATAAC
CAGTGAAACAGCATTGCTGTCACTTGGTCGTGGCAGCCCGGACCGACGATGAAGCATGTTTAGCTG
GCCCAAATGTTGCTGGATAGTTTTTACTGCCAGACCGCGCGCTTGAAGATATAGAAGATAATCGCG
AACATCTTCAGGTTCTGCGGGAAACCATTTCCGGTTATTCAACTTGCACCATGCCGCCCACGACCG
GCAAACGGACAGAAGCATTTTCCAGGTATGCTCAGAAAACGCCTGGCGATCCCTGAACATGTCCAT
CAGGTTCTTGCGAACCTCATCACTCGTTGCATCGACCGGTAATGCAGGCAAATTTTGGTGTACGGT
CAGTAAATTGGACATGGTGGCTACGTAATAACTTCGTATATGGTTTCTTATACGAAGTTATGCGGC
CGCTTTACGAGGGTAGGAAGTGGTACGGAAAGTTGGTATAAGACAAAAGTGTTGTGGAATTGCTCC
AGGCGATCTGACGGTTCACTAAACGAGCTCTGCTTTTATAGGCGCCCACCGTACACGCCTAAAGCT
TATACGTTCTCTATCACTGATAGGGAGTAAACTGGATATACGTTCTCTATCACTGATAGGGAGTAA
ACTGTAGATACGTTCTCTATCACTGATAGGGAGTAAACTGGTCATACGTTCTCTATCACTGATAGG
GAGTAAACTCCTTATACGTTCTCTATCACTGATAGGGAGTAAAGTCTGCATACGTTCTCTATCACT
GATAGGGAGTAAACTCTTCATACGTTCTCTATCACTGATAGGGAGTAAACTCGCGGCCGCAGAGAA
ATGTTCTGGCACCTGCACTTGCACTGGGGACAGCCTATTTTGCTAGTTTGTTTTGTTTCGTTTTGT
TTTGATGGAGAGCGTATGTTAGTACTATCGATTCACACAAAAAACCAACACACAGATGTAATGAAA
ATAAAGATATTTTATTGGATCTGCGATCGCTCCGGTGCCCGTCAGTGGGCAGAGCGCACATCGCCC
ACAGTCCCCGAGAAGTTGGGGGGAGGGGTCGGCAATTGAACGGGTGCCTAGAGAAGGTGGCGCGGG
GTAAACTGGGAAAGTGATGTCGTGTACTGGCTCCGCCTTTTTCCCGAGGGTGGGGGAGAACCGTAT
GTAAGTGCAGTAGTCGCCGTGAACGTTCTTTTTCGCAACGGGTTTGCCGCCAGAACACAGCTGAAG
CTTCGAGGGGCTCGCATCTCTCCTTCACGCGCCCGCCGCCCTACCTGAGGCCGCCATCCACGCCGG
TTGAGTCGCGTTCTGCCGCCTCCCGCCTGTGGTGCCTCCTGAACTGCGTCCGCCGTCTAGGTAAGT
TTAAAGCTCAGGTCGAGACCGGGCCTTTGTCCGGCGCTCCCTTGGAGCCTACCTAGACTCAGCCGG
CTCTCCACGCTTTGCCTGACCCTGCTTGCTCAACTCTACGTCTTTGTTTCGTTTTCTGTTCTGCGC
CGTTACAGATCCAAGCTGTGACCGGCGCCTACGCTAGCGGATCCGCCGCCACCATGTCTAGACTGG
ACAAGAGCAAAGTCATAAACTCTGCTCTGGAATTACTCAATGGAGTCGGTATCGAAGGCCTGACGA
CAAGGAAACTCGCTCAAAAGCTGGGAGTTGAGCAGCCTACCCTGTACTGGCACGTGAAGAACAAGC
GGGCCCTGCTCGATGCCCTGCCAATCGAGATGCTGGACAGGCATCATACCCACTCCTGCCCCCTGG
AAGGCGAGTCATGGCAAGACTTTCTGCGGAACAACGCCAAGTCATACCGCTGTGCTCTTCTCTCAC
ATCGCGACGGGGCTAAAGTGCATCTCGGCACCCGCCCAACAGAGAAACAGTACGAAACCCTGGAAA
ATCAGCTCGCGTTCCTGTGTCAGCAAGGCTTCTCCCTGGAGAACGCACTGTACGCTCTGTCCGCCG
TGGGCCACTTTACACTGGGCTGCGTATTGGAGGAACAGGAGCATCAAGTAGCAAAAGAGGAAAGAG
AGACACCTACCACCGATTCTATGCCCCCACTTCTGAAACAAGCAATTGAGCTGTTCGACCGGCAGG
GAGCCGAACCTGCCTTCCTTTTCGGCCTGGAACTAATCATATGTGGCCTGGAGAAACAGCTAAAGT
GCGAAAGCGGCGGGCCGACCGACGCCCTTGACGATTTTGACTTAGACATGCTCCCAGCCGATGCCC
TTGACGACTTTGACCTTGATATGCTGCCTGCTGACGCTCTTGACGATTTTGACCTTGACATGCTCC
CCGGGGGATCCGGAGCTACTAACTTCAGCCTGCTGAAGCAGGCTGGAGACGTGGAGGAGAACCCTG
GACCTATGACAGAATATAAACCTACTGTCAGACTGGCAACTCGAGACGACGTCCCTAGGGCCGTGA
GAACATTGGCTGCCGCTTTCGCGGATTATCCCGCTACACGCCACACAGTTGATCCTGATAGACATA
TTGAACGGGTTACAGAATTGCAAGAACTTTTTTTGACCAGGGTAGGATTGGACATCGGTAAAGTTT
GGGTCGCCGACGACGGGGCTGCAGTGGCAGTGTGGACGACTCCGGAGAGCGTTGAGGCCGGGGCTG
TATTTGCAGAAATTGGTCCCCGAATGGCTGAGCTTAGTGGCTCTCGTCTCGCGGCTCAGCAACAAA
TGGAAGGACTCCTCGCCCCTCACCGCCCTAAAGAACCAGCTTGGTTCCTCGCTACTGTGGGCGTTA
GCCCCGATCATCAGGGAAAGGGCCTTGGTTCCGCGGTGGTATTGCCCGGAGTAGAAGCCGCAGAAC
GAGCCGGAGTGCCAGCCTTTCTTGAAACGTCAGCGCCAAGGAATTTGCCCTTCTATGAACGGCTCG
GATTTACAGTTACTGCTGACGTTGAAGTACCCGAGGGCCCACGGACGTGGTGCATGACGCGAAAAC
CCGGCGCTTGAGTTTAAACCGCTGATCAGCCTCGACTGTGCCTTCTAGTTGCCAGCCATCTGTTGT
TTGCCCCTCCCCCGTGCCTTCCTTGACCCTGGAAGGTGCCACTCCCACTGTCCTTTCCTAATAAAA
TGAGGAAATTGCATCGCATTGTCTGAGTAGGTGTCATTCTATTCTGGGGGGTGGGGTGGGGCAGGA
CAGCAAGGGGGAGGATTGGGAAGACAATAGCAGGCATGCTGGGGATGCGGTGGGCTCTATGGCTTC
TGAGGCGGAAAGAACCAGCTGGGGCTCGACTAGAGCTTGCGGAACCCTTAGTTTAAACGGGCCCTT
AATTAATCGATGTAGGATGTTGCCCCTCCTGACGCGGTAGGAGAAGGGGAGGGTGCCCTGCATGTC
TGCCGCTGCTCTTGCTCTTGCCGCTGCTGAGGAGGGGGGCGCATCTGCCGCAGCACCGGATGCATC
TGGGAAAAGCAAAAAAGGGGCTCGTCCCTGTTTCCGGAGGAATTTGCAAGCGGGGTCTTGCATGAC
GGGGAGGCAAACCCCCGTTCGCCGCAGTCCGGCCGGCCCGAGACTCGAACCGGGGGTCCTGCGACT
CAACCCTTGGAAAATAACCCTCCGGCTACAGGGAGCGAGCCACTTAATGCTTTCGCTTTCCAGCCT
AACCGCTTACGCCGCGCGCGGCCAGTGGCCAAAAAAGCTAGCGCAGCAGCCGCCGCGCCTGGAAGG
AAGCCAAAAGGAGCGCTCCCCCGTTGTCTGACGTCGCACACCTGGGTTCGACACGCGGGCGGTAAC
CGCATGGATCACGGCGGACGGCCGGATCCGGGGTTCGAACCCCGGTCGTCCGCCATGATACCCTTG
CGAATTTATCCACCAGACCACGGAAGAGTGCCCGCTTACAGGCTCTCCTTTTGCACGGTCTAGAGC
GTCAACGACTGCGCACGCCTCACCGGCCAGAGCGTCCCGACCATGGAGCACTTTTTGCCGCTGCGC
AACATCTGGAACCGCGTCCGCGACTTTCCGCGCGCCTCCACCACCGCCGCCGGCATCACCTGGATG
TCCAGGTACATCTACGGATTACGGGGCCCATTGGTATG
SEQ ID NO: 21 (reverse tetracycline-controlled transactivator mutant)
ATGTCTAGACTGGACAAGAGCAAAGTCATAAACTCTGCTCTGGAATTACTCAATGGAGTCGGTATC
GAAGGCCTGACGACAAGGAAACTCGCTCAAAAGCTGGGAGTTGAGCAGCCTACCCTGTACTGGCAC
GTGAAGAACAAGCGGGCCCTGCTCGATGCCCTGCCAATCGAGATGCTGGACAGGCATCATACCCAC
TCCTGCCCCCTGGAAGGCGAGTCATGGCAAGACTTTCTGCGGAACAACGCCAAGTCATACCGCTGT
GCTCTTCTCTCACATCGCGACGGGGCTAAAGTGCATCTCGGCACCCGCCCAACAGAGAAACAGTAC
GAAACCCTGGAAAATCAGCTCGCGTTCCTGTGTCAGCAAGGCTTCTCCCTGGAGAACGCACTGTAC
GCTCTGTCCGCCGTGGGCCACTTTACACTGGGCTGCGTATTGGAGGAACAGGAGCATCAAGTAGCA
AAAGAGGAAAGAGAGACACCTACCACCGATTCTATGCCCCCACTTCTGAAACAAGCAATTGAGCTG
TTCGACCGGCAGGGAGCCGAACCTGCCTTCCTTTTCGGCCTGGAACTAATCATATGTGGCCTGGAG
AAACAGCTAAAGTGCGAAAGCGGCGGGCCGACCGACGCCCTTGACGATTTTGACTTAGACATGCTC
CCAGCCGATGCCCTTGACGACTTTGACCTTGATATGCTGCCTGCTGACGCTCTTGACGATTTTGAC
CTTGACATGCTCCCCGGG
SEQ ID NO: 22 (Tet inducible promoter sequence)
GAGTTTACTCCCTATCAGTGATAGAGAACGTATGAAGAGTTTACTCCCTATCAGTGATAGAGAACG
TATGCAGACTTTACTCCCTATCAGTGATAGAGAACGTATAAGGAGTTTACTCCCTATCAGTGATAG
AGAACGTATGACCAGTTTACTCCCTATCAGTGATAGAGAACGTATCTACAGTTTACTCCCTATCAG
TGATAGAGAACGTATATCCAGTTTACTCCCTATCAGTGATAGAGAACGTATAAGCTTTAGGCGTGT
ACGGTGGGCGCCTATAAAAGCAGAGCTCGTTTAGTGAACCGTCAGATCGCCTGGAGCAATTCCACA
ACACTTTTGTCTTATACCAACTTTCCGTACCACTTCCTACCCTCGTAAA
SEQ ID NO: 23 (STXC0034)
GGTACCCAACTCCATGCTTAACAGTCCCCAGGTACAGCCCACCCTGCGTCGCAACCAGGAACAGCT
CTACAGCTTCCTGGAGCGCCACTCGCCCTACTTCCGCAGCCACAGTGCGCAGATTAGGAGCGCCAC
TTCTTTTTGTCACTTGAAAAACATGTAAAAATAATGTACTAGGAGACACTTTCAATAAAGGCAAAT
GTTTTTATTTGTACACTCTCGGGTGATTATTTACCCCCCACCCTTGCCGTCTGCGCCGTTTAAAAA
TCAAAGGGGTTCTGCCGCGCATCGCTATGCGCCACTGGCAGGGACACGTTGCGATACTGGTGTTTA
GTGCTCCACTTAAACTCAGGCACAACCATCCGCGGCAGCTCGGTGAAGTTTTCACTCCACAGGCTG
CGCACCATCACCAACGCGTTTAGCAGGTCGGGCGCCGATATCTTGAAGTCGCAGTTGGGGCCTCCG
CCCTGCGCGCGCGAGTTGCGATACACAGGGTTGCAGCACTGGAACACTATCAGCGCCGGGTGGTGC
ACGCTGGCCAGCACGCTCTTGTCGGAGATCAGATCCGCGTCCAGGTCCTCCGCGTTGCTCAGGGCG
AACGGAGTCAACTTTGGTAGCTGCCTTCCCAAAAAGGGTGCATGCCCAGGCTTTGAGTTGCACTCG
CACCGTAGTGGCATCAGAAGGTGACCGTGCCCGGTCTGGGCGTTAGGATACAGCGCCTGCATGAAA
GCCTTGATCTGCTTAAAAGCCACCTGAGCCTTTGCGCCTTCAGAGAAGAACATGCCGCAAGACTTG
CCGGAAAACTGATTGGCCGGACAGGCCGCGTCATGCACGCAGCACCTTGCGTCGGTGTTGGAGATC
TGCACCACATTTCGGCCCCACCGGTTCTTCACGATCTTGGCCTTGCTAGACTGCTCCTTCAGCGCG
CGCTGCCCGTTTTCGCTCGTCACATCCATTTCAATCACGTGCTCCTTATTTATCATAATGCTCCCG
TGTAGACACTTAAGCTCGCCTTCGATCTCAGCGCAGCGGTGCAGCCACAACGCGCAGCCCGTGGGC
TCGTGGTGCTTGTAGGTTACCTCTGCAAACGACTGCAGGTACGCCTGCAGGAATCGCCCCATCATC
GTCACAAAGGTCTTGTTGCTGGTGAAGGTCAGCTGCAACCCGCGGTGCTCCTCGTTTAGCCAGGTC
TTGCATACGGCCGCCAGAGCTTCCACTTGGTCAGGCAGTAGCTTGAAGTTTGCCTTTAGATCGTTA
TCCACGTGGTACTTGTCCATCAACGCGCGCGCAGCCTCCATGCCCTTCTCCCACGCAGACACGATC
GGCAGGCTCAGCGGGTTTATCACCGTGCTTTCACTTTCCGCTTCACTGGACTCTTCCTTTTCCTCT
TGCGTCCGCATACCCCGCGCCACTGGGTCGTCTTCATTCAGCCGCCGCACCGTGCGCTTACCTCCC
TTGCCGTGCTTGATTAGCACCGGTGGGTTGCTGAAACCCACCATTTGTAGCGCCACATCTTCTCTT
TCTTCCTCGCTGTCCACGATCACCTCTGGGGATGGCGGGCGCTCGGGCTTGGGAGAGGGGCGCTTC
TTTTTCTTTTTGGACGCAATGGCCAAATCCGCCGTCGAGGTCGATGGCCGCGGGCTGGGTGTGCGC
GGCACCAGCGCATCTTGTGACGAGTCTTCTTCGTCCTCGGACTCGAGACGCCGCCTCAGCCGCTTT
TTTGGGGGCGCGCGGGGAGGCGGCGGCGACGGCGACGGGGACGACACGTCCTCCATGGTTGGTGGA
CGTCGCGCCGCACCGCGTCCGCGCTCGGGGGTGGTTTCGCGCTGCTCCTCTTCCCGACTGGCCATT
TCCTTCTCCTATAGGCAGAAAAAGATCATGGAGTCAGTCGAGAAGGAGGACAGCCTAACCGCCCCC
TTTGAGTTCGCCACCACCGCCTCCACCGATGCCGCCAACGCGCCTACCACCTTCCCCGTCGAGGCA
CCCCCGCTTGAGGAGGAGGAAGTGATTATCGAGCAGGACCCAGGTTTTGTAAGCGAAGACGACGAG
GATCGCTCAGTACCAACAGAGGATAAAAAGCAAGACCAGGACGACGCAGAGGCAAACGAGGAACAA
GTCGGGCGGGGGGACCAAAGGCATGGCGACTACCTAGATGTGGGAGACGACGTGCTGTTGAAGCAT
CTGCAGCGCCAGTGCGCCATTATCTGCGACGCGTTGCAAGAGCGCAGCGATGTGCCCCTCGCCATA
GCGGATGTCAGCCTTGCCTACGAACGCCACCTGTTCTCACCGCGCGTACCCCCCAAACGCCAAGAA
AACGGCACATGCGAGCCCAACCCGCGCCTCAACTTCTACCCCGTATTTGCCGTGCCAGAGGTGCTT
GCCACCTATCACATCTTTTTCCAAAACTGCAAGATACCCCTATCCTGCCGTGCCAACCGCAGCCGA
GCGGACAAGCAGCTGGCCTTGCGGCAGGGCGCTGTCATACCTGATATCGCCTCGCTCGACGAAGTG
CCAAAAATCTTTGAGGGTCTTGGACGCGACGAGAAACGCGCGGCAAACGCTCTGCAACAAGAAAAC
AGCGAAAATGAAAGTCACTGTGGAGTGCTGGTGGAACTTGAGGGTGACAACGCGCGCCTAGCCGTG
CTGAAACGCAGCATCGAGGTCACCCACTTTGCCTACCCGGCACTTAACCTACCCCCCAAGGTTATG
AGCACAGTCATGAGCGAGCTGATCGTGCGCCGTGCACGACCCCTGGAGAGGGATGCAAACTTGCAA
GAACAAACCGAGGAGGGCCTACCCGCAGTTGGCGATGAGCAGCTGGCGCGCTGGCTTGAGACGCGC
GAGCCTGCCGACTTGGAGGAGCGACGCAAGCTAATGATGGCCGCAGTGCTTGTTACCGTGGAGCTT
GAGTGCATGCAGCGGTTCTTTGCTGACCCGGAGATGCAGCGCAAGCTAGAGGAAACGTTGCACTAC
ACCTTTCGCCAGGGCTACGTGCGCCAGGCCTGCAAAATTTCCAACGTGGAGCTCTGCAACCTGGTC
TCCTACCTTGGAATTTTGCACGAAAACCGCCTCGGGCAAAACGTGCTTCATTCCACGCTCAAGGGC
GAGGCGCGCCGCGACTACGTCCGCGACTGCGTTTACTTATTTCTGTGCTACACCTGGCAAACGGCC
ATGGGCGTGTGGCAGCAATGCCTGGAGGAGCGCAACCTAAAGGAGCTGCAGAAGCTGCTAAAGCAA
AACTTGAAGGACCTATGGACGGCCTTCAACGAGCGCTCCGTGGCCGCGCACCTGGCGGACATTATC
TTCCCCGAACGCCTGCTTAAAACCCTGCAACAGGGTCTGCCAGACTTCACCAGTCAAAGCATGTTG
CAAAACTTTAGGAACTTTATCCTAGAGCGTTCAGGAATTCTGCCCGCCACCTGCTGTGCGCTTCCT
AGCGACTTTGTGCCCATTAAGTACCGTGAATGCCCTCCGCCGCTTTGGGGTCACTGCTACCTTCTG
CAGCTAGCCAACTACCTTGCCTACCACTCCGACATCATGGAAGACGTGAGCGGTGACGGCCTACTG
GAGTGTCACTGTCGCTGCAACCTATGCACCCCGCACCGCTCCCTGGTCTGCAATTCGCAACTGCTT
AGCGAAAGTCAAATTATCGGTACCTTTGAGCTGCAGGGTCCCTCGCCTGACGAAAAGTCCGCGGCT
CCGGGGTTGAAACTCACTCCGGGGCTGTGGACGTCGGCTTACCTTCGCAAATTTGTACCTGAGGAC
TACCACGCCCACGAGATTAGGTTCTACGAAGACCAATCCCGCCCGCCAAATGCGGAGCTTACCGCC
TGCGTCATTACCCAGGGCCACATCCTTGGCCAATTGCAAGCCATCAACAAAGCCCGCCAAGAGTTT
CTGCTACGAAAGGGACGGGGGGTTTACCTGGACCCCCAGTCCGGCGAGGAGCTCAACCCAATCCCC
CCGCCGCCGCAGCCCTATCAGCAGCCGCGGGCCCTTGCTTCCCAGGATGGCACCCAAAAAGAAGCT
GCAGCTGCCGCCGCCGCCACCCACGGACGAGGAGGAATACTGGGACAGTCAGGCAGAGGAGGTTTT
GGACGAGGAGGAGGAGATGATGGAAGACTGGGACAGCCTAGACGAAGCTTCCGAGGCCGAAGAGGT
GTCAGACGAAACACCGTCACCCTCGGTCGCATTCCCCTCGCCGGCGCCCCAGAAATTGGCAACCGT
TCCCAGCATCGCTACAACCTCCGCTCCTCAGGCGCCGCCGGCACTGCCTGTTCGCCGACCCAACCG
TAGATGGGACACCACTGGAACCAGGGCCGGTAAGTCTAAGCAGCCGCCGCCGTTAGCCCAAGAGCA
ACAACAGCGCCAAGGCTACCGCTCGTGGCGCGGGCACAAGAACGCCATAGTTGCTTGCTTGCAAGA
CTGTGGGGGCAACATCTCCTTCGCCCGCCGCTTTCTTCTCTACCATCACGGCGTGGCCTTCCCCCG
TAACATCCTGCATTACTACCGTCATCTCTACAGCCCCTACTGCACCGGCGGCAGCGGCAGCGGCAG
CAACAGCAGCGGTCACACAGAAGCAAAGGCGACCGGATAGCAAGACTCTGACAAAGCCCAAGAAAT
CCACAGCGGCGGCAGCAGCAGGAGGAGGAGCGCTGCGTCTGGCGCCCAACGAACCCGTATCGACCC
GCGAGCTTAGAAATAGGATTTTTCCCACTCTGTATGCTATATTTCAACAAAGCAGGGGCCAAGAAC
AAGAGCTGAAAATAAAAAACAGGTCTCTGCGCTCCCTCACCCGCAGCTGCCTGTATCACAAAAGCG
AAGATCAGCTTCGGCGCACGCTGGAAGACGCGGAGGCTCTCTTCAGCAAATACTGCGCGCTGACTC
TTAAGGACTAGTTTCGCGCCCTTTCTCAAATTTAAGCGCGAAAACTACGTCATCTCCAGCGGCCAC
ACCCGGCGCCAGCACCTGTCGTCAGCGCCATTATGAGCAAGGAAATTCCCACGCCCTACATGTGGA
GTTACCAGCCACAAATGGGACTTGCGGCTGGAGCTGCCCAAGACTACTCAACCCGAATAAACTACA
TGAGCGCGGGACCCCACATGATATCCCGGGTCAACGGAATCCGCGCCCACCGAAACCGAATTCTCC
TCGAACAGGCGGCTATTACCACCACACCTCGTAATAACCTTAATCCCCGTAGTTGGCCCGCTGCCC
TGGTGTACCAGGAAAGTCCCGCTCCCACCACTGTGGTACTTCCCAGAGACGCCCAGGCCGAAGTTC
AGATGACTAACTCAGGGGCGCAGCTTGCGGGCGGCTTTCGTCACAGGGTGCGGTCGCCCGGGCGTT
TTAGGGCGGAGTAACTTGCATGTATTGGGAATTGTAGTTTTTTTAAAATGGGAAGTGACGTATCGT
GGGAAAACGGAAGTGAAGATTTGAGGAAGTTGTGGGTTTTTTGGCTTTCGTTTCTGGGCGTAGGTT
CGCGTGCGGTTTTCTGGGTGTTTTTTGTGGACTTTAACCGTTACGTCATTTTTTAGTCCTATATAT
ACTCGCTCTGTACTTGGCCCTTTTTACACTGTGACTGATTGAGCTGGTGCCGTGTCGAGTGGTGTT
TTTTAATAGGTTTTTTTACTGGTAAGGCTGACTGTTATGGCTGCCGCTGTGGAAGCGCTGTATGTT
GTTCTGGAGCGGGAGGGTGCTATTTTGCCTAGGCAGGAGGGTTTTTCAGGTGTTTATGTGTTTTTC
TCTCCTATTAATTTTGTTATACCTCCTATGGGGGCTGTAATGTTGTCTCTACGCCTGCGGGTATGT
ATTCCCCCGGGCTATTTCGGTCGCTTTTTAGCACTGACCGATGTTAACCAACCTGATGTGTTTACC
GAGTCTTACATTATGACTCCGGACATGACCGAGGAACTGTCGGTGGTGCTTTTTAATCACGGTGAC
CAGTTTTTTTACGGTCACGCCGGCATGGCCGTAGTCCGTCTTATGCTTATAAGGGTTGTTTTTCCT
GTTGTAAGACAGGCTTCTAATGTTTAAATGTTTTTTTTTTTGTTATTTTATTTTGTGTTTAATGCA
GGAACCCGCAGACATGTTTGAGAGAAAAATGGTGTCTTTTTCTGTGGTGGTTCCGGAACTTACCTG
CCTTTATCTGCATGAGCATGACTACGATGTGCTTGCTTTTTTGCGCGAGGCTTTGCCTGATTTTTT
GAGCAGCACCTTGCATTTTATATCGCCGCCCATGCAACAAGCTTACATAGGGGCTACGCTGGTTAG
CATAGCTCCGAGTATGCGTGTCATAATCAGTGTGGGTTCTTTTGTCATGGTTCCTGGCGGGGAAGT
GGCCGCGCTGGTCCGTGCAGACCTGCACGATTATGTTCAGCTGGCCCTGCGAAGGGACCTACGGGA
TCGCGGTATTTTTGTTAATGTTCCGCTTTTGAATCTTATACAGGTCTGTGAGGAACCTGAATTTTT
GCAATCATGATTCGCTGCTTGAGGCTGAAGGTGGAGGGCGCTCTGGAGCAGATTTTTACAATGGCC
GGACTTAATATTCGGGATTTGCTTAGAGACATATTGATAAGGTGGCGAGATGAAAATTATTTGGGC
ATGGTTGAAGGTGCTGGAATGTTTATAGAGGAGATTCACCCTGAAGGGTTTAGCCTTTACGTCCAC
TTGGACGTGAGGGCAGTTTGCCTTTTGGAAGCCATTGTGCAACATCTTACAAATGCCATTATCTGT
TCTTTGGCTGTAGAGTTTGACCACGCCACCGGAGGGGAGCGCGTTCACTTAATAGATCTTCATTTT
GAGGTTTTGGATAATCTTTTGGAATAAAAAAAAAAAAACATGGTTCTTCCAGCTCTTCCCGCTCCT
CCCGTGTGTGACTCGCAGAACGAATGTGTAGGTTGGCTGGGTGTGGCTTATTCTGCGGTGGTGGAT
GTTATCAGGGCAGCGGCGCATGAAGGAGTTTACATAGAACCCGAAGCCAGGGGGCGCCTGGATGCT
TTGAGAGAGTGGATATACTACAACTACTACACAGAGCGAGCTAAGCGACGAGACCGGAGACGCAGA
TCTGTTTGTCACGCCCGCACCTGGTTTTGCTTCAGGAAATATGACTACGTCCGGCGTTCCATTTGG
CATGACACTACGACCAACACGATCTCGGTTGTCTCGGCGCACTCCGTACAGTAGGGATCGCCTACC
TCCTTTTGAGACAGAGACCCGCGCTACCATACTGGAGGATCATCCGCTGCTGCCCGAATGTAACAC
TTTGACAATGCACAACGTGAGTTACGTGCGAGGTCTTCCCTGCAGTGTGGGATTTACGCTGATTCA
GGAATGGGTTGTTCCCTGGGATATGGTTCTGACGCGGGAGGAGCTTGTAATCCTGAGGAAGTGTAT
GCACGTGTGCCTGTGTTGTGCCAACATTGATATCATGACGAGCATGATGATCCATGGTTACGAGTC
CTGGGCTCTCCACTGTCATTGTTCCAGTCCCGGTTCCCTGCAGTGCATAGCCGGCGGGCAGGTTTT
GGCCAGCTGGTTTAGGATGGTGGTGGATGGCGCCATGTTTAATCAGAGGTTTATATGGTACCGGGA
GGTGGTGAATTACAACATGCCAAAAGAGGTAATGTTTATGTCCAGCGTGTTTATGAGGGGTCGCCA
CTTAATCTACCTGCGCTTGTGGTATGATGGCCACGTGGGTTCTGTGGTCCCCGCCATGAGCTTTGG
ATACAGCGCCTTGCACTGTGGGATTTTGAACAATATTGTGGTGCTGTGCTGCAGTTACTGTGCTGA
TTTAAGTGAGATCAGGGTGCGCTGCTGTGCCCGGAGGACAAGGCGTCTCATGCTGCGGGCGGTGCG
AATCATCGCTGAGGAGACCACTGCCATGTTGTATTCCTGCAGGACGGAGCGGCGGCGGCAGCAGTT
TATTCGCGCGCTGCTGCAGCACCACCGCCCTATCCTGATGCACGATTATGACTCTACCCCCATGTA
GGCGTGGACTTCCCCTTCGCCGCCCGTTGAGCAACCGCAAGTTGGACAGCAGCCTGTGGCTCAGCA
GCTGGACAGCGACATGAACTTAAGCGAGCTGCCCGGGGAGTTTATTAATATCACTGATGAGCGTTT
GGCTCGACAGGAAACCGTGTGGAATATAACACCTAAGAATATGTCTGTTACCCATGATATGATGCT
TTTTAAGGCCAGCCGGGGAGAAAGGACTGTGTACTCTGTGTGTTGGGAGGGAGGTGGCAGGTTGAA
TACTAGGGTTCTGTGAGTTTGATTAAGGTACGGTGATCAATATAAGCTATGTGGTGGTGGGGCTAT
ACTACTGAATGAAAAATGACTTGAAATTTTCTGCAATTGAAAAATAAACACGTTGAAACATAACAT
GCAACAGGTTCACGATTCTTTATTCCTGGGCAATGTAGGAGAAGGTGTAAGAGTTGGTAGCAAAAG
TTTCAGTGGTGTATTTTCCACTTTCCCAGGACCATGTAAAAGACATAGAGTAAGTGCTTACCTCGC
TAGTTTCTGTGGATTCACTAGAATCGATGTCGACGGGCACTCTTCCGTGGTCTGGTGGATAAATTC
GCAAGGGTATCATGGCGGACGAACCGGTACCGAACCCCGGATCCGGCCGTCCGCCGTGATCCATGC
GGTTACCGCCCGCGTGTCGAACCCAGGTGTGCGACGTCAGACAACGGGGGAGCGCTCCTTTTTCAT
ATGATCAGCTCACTCAAAGGCGGTAATACGGTTATCCACAGAATCAGGGGATAACGCAGGAAAGAA
CATGTGAGCAAAAGGCCAGCAAAAGGCCAGGAACCGTAAAAAGGCCGCGTTGCTGGCGTTTTTCCA
TAGGCTCCGCCCCCCTGACGAGCATCACAAAAATCGACGCTCAAGTCAGAGGTGGCGAAACCCGAC
AGGACTATAAAGATACCAGGCGTTTCCCCCTGGAAGCTCCCTCGTGCGCTCTCCTGTTCCGACCCT
GCCGCTTACCGGATACCTGTCCGCCTTTCTCCCTTCGGGAAGCGTGGCGCTTTCTCATAGCTCACG
CTGTAGGTATCTCAGTTCGGTGTAGGTCGTTCGCTCCAAGCTGGGCTGTGTGCACGAACCCCCCGT
TCAGCCCGACCGCTGCGCCTTATCCGGTAACTATCGTCTTGAGTCCAACCCGGTAAGACACGACTT
ATCGCCACTGGCAGCAGCCACTGGTAACAGGATTAGCAGAGCGAGGTATGTAGGCGGTGCTACAGA
GTTCTTGAAGTGGTGGCCTAACTACGGCTACACTAGAAGAACAGTATTTGGTATCTGCGCTCTGCT
GAAGCCAGTTACCTTCGGAAAAAGAGTTGGTAGCTCTTGATCCGGCAAACAAACCACCGCTGGTAG
CGGTGGTTTTTTTGTTTGCAAGCAGCAGATTACGCGCAGAAAAAAAGGATCTCAAGAAGATCCTTT
GATCTTTTCTACGGGGTCTGACGCTCAGTGGAACGAAAACTCACGTTAAGGGATTTTGGTCATGAG
ATTATCAAAAAGGATCTTCACCTAGATCCTTTTAAATTAAAAATGAAGTTTTAAATCAATCTAAAG
TATATATGAGTAAACTTGGTCTGACAGTTACCAATGCTTAATCAGTGAGGCACCTATCTCAGCGAT
CTGTCTATTTCGTTCATCCATAGTTGCCTGACTCCCCGTCGTGTAGATAACTACGATACGGGAGGG
CTTACCATCTGGCCCCAGTGCTGCAATGATACCGCGAGACCCACGCTCACCGGCTCCAGATTTATC
AGCAATAAACCAGCCAGCCGGAAGGGCCGAGCGCAGAAGTGGTCCTGCAACTTTATCCGCCTCCAT
CCAGTCTATTAATTGTTGCCGGGAAGCTAGAGTAAGTAGTTCGCCAGTTAATAGTTTGCGCAACGT
TGTTGCCATTGCTACAGGCATCGTGGTGTCACGCTCGTCGTTTGGTATGGCTTCATTCAGCTCCGG
TTCCCAACGATCAAGGCGAGTTACATGATCCCCCATGTTGTGCAAAAAAGCGGTTAGCTCCTTCGG
TCCTCCGATCGTTGTCAGAAGTAAGTTGGCCGCAGTGTTATCACTCATGGTTATGGCAGCACTGCA
TAATTCTCTTACTGTCATGCCATCCGTAAGATGCTTTTCTGTGACTGGTGAGTACTCAACCAAGTC
ATTCTGAGAATAGTGTATGCGGCGACCGAGTTGCTCTTGCCCGGCGTCAATACGGGATAATACCGC
GCCACATAGCAGAACTTTAAAAGTGCTCATCATTGGAAAACGTTCTTCGGGGCGAAAACTCTCAAG
GATCTTACCGCTGTTGAGATCCAGTTCGATGTAACCCACTCGTGCACCCAACTGATCTTCAGCATC
TTTTACTTTCACCAGCGTTTCTGGGTGAGCAAAAACAGGAAGGCAAAATGCCGCAAAAAAGGGAAT
AAGGGCGACACGGAAATGTTGAATACTCATACTCTTCCTTTTTCAATATTATTGAAGCATTTATCA
GGGTTATTGTCTCATGAGCGGATACATATTTGAATGTATTTAGAAAAATAAACAAATAGGGGTTCC
GCGCACATTTCCCCGAAAAGTGCCACCTAAATTGTAAGCGTTAATATTTTGTTAAAATTCGCGTTA
AATTTTTGTTAAATCAGCTCATTTTTTAACCAATAGGCCGAAATCGGCAAAATCCCTTATAAATCA
AAAGAATAGACCGAGATAGGGTTGAGTGTTGTTCCAGTTTGGAACAAGAGTCCACTATTAAAGAAC
GTGGACTCCAACGTCAAAGGGCGAAAAACCGTCTATCAGGGCGATGGCCCACTACGTGAACCATCA
CCCTAATCAAGTTTTTTGGGGTCGAGGTGCCGTAAAGCACTAAATCGGAACCCTAAAGGGAGCCCC
CGATTTAGAGCTTGACGGGGAAAGCCGGCGAACGTGGCGAGAAAGGAAGGGAAGAAAGCGAAAGGA
GCGGGCGCTAGGGCGCTGGCAAGTGTAGCGGTCACGCTGCGCGTAACCACCACACCCGCCGCGCTT
AATGCGCCGCTACAGGGCGCGATGGATCC
SEQ ID NO: 24 (STXC0036)
GGTACCCAACTCCATGCTTAACAGTCCCCAGGTACAGCCCACCCTGCGTCGCAACCAGGAACAGCT
CTACAGCTTCCTGGAGCGCCACTCGCCCTACTTCCGCAGCCACAGTGCGCAGATTAGGAGCGCCAC
TTCTTTTTGTCACTTGAAAAACATGTAAAAATAATGTACTAGGAGACACTTTCAATAAAGGCAAAT
GTTTTTATTTGTACACTCTCGGGTGATTATTTACCCCCCACCCTTGCCGTCTGCGCCGTTTAAAAA
TCAAAGGGGTTCTGCCGCGCATCGCTATGCGCCACTGGCAGGGACACGTTGCGATACTGGTGTTTA
GTGCTCCACTTAAACTCAGGCACAACCATCCGCGGCAGCTCGGTGAAGTTTTCACTCCACAGGCTG
CGCACCATCACCAACGCGTTTAGCAGGTCGGGCGCCGATATCTTGAAGTCGCAGTTGGGGCCTCCG
CCCTGCGCGCGCGAGTTGCGATACACAGGGTTGCAGCACTGGAACACTATCAGCGCCGGGTGGTGC
ACGCTGGCCAGCACGCTCTTGTCGGAGATCAGATCCGCGTCCAGGTCCTCCGCGTTGCTCAGGGCG
AACGGAGTCAACTTTGGTAGCTGCCTTCCCAAAAAGGGTGCATGCCCAGGCTTTGAGTTGCACTCG
CACCGTAGTGGCATCAGAAGGTGACCGTGCCCGGTCTGGGCGTTAGGATACAGCGCCTGCATGAAA
GCCTTGATCTGCTTAAAAGCCACCTGAGCCTTTGCGCCTTCAGAGAAGAACATGCCGCAAGACTTG
CCGGAAAACTGATTGGCCGGACAGGCCGCGTCATGCACGCAGCACCTTGCGTCGGTGTTGGAGATC
TGCACCACATTTCGGCCCCACCGGTTCTTCACGATCTTGGCCTTGCTAGACTGCTCCTTCAGCGCG
CGCTGCCCGTTTTCGCTCGTCACATCCATTTCAATCACGTGCTCCTTATTTATCATAATGCTCCCG
TGTAGACACTTAAGCTCGCCTTCGATCTCAGCGCAGCGGTGCAGCCACAACGCGCAGCCCGTGGGC
TCGTGGTGCTTGTAGGTTACCTCTGCAAACGACTGCAGGTACGCCTGCAGGAATCGCCCCATCATC
GTCACAAAGGTCTTGTTGCTGGTGAAGGTCAGCTGCAACCCGCGGTGCTCCTCGTTTAGCCAGGTC
TTGCATACGGCCGCCAGAGCTTCCACTTGGTCAGGCAGTAGCTTGAAGTTTGCCTTTAGATCGTTA
TCCACGTGGTACTTGTCCATCAACGCGCGCGCAGCCTCCATGCCCTTCTCCCACGCAGACACGATC
GGCAGGCTCAGCGGGTTTATCACCGTGCTTTCACTTTCCGCTTCACTGGACTCTTCCTTTTCCTCT
TGCGTCCGCATACCCCGCGCCACTGGGTCGTCTTCATTCAGCCGCCGCACCGTGCGCTTACCTCCC
TTGCCGTGCTTGATTAGCACCGGTGGGTTGCTGAAACCCACCATTTGTAGCGCCACATCTTCTCTT
TCTTCCTCGCTGTCCACGATCACCTCTGGGGATGGCGGGCGCTCGGGCTTGGGAGAGGGGCGCTTC
TTTTTCTTTTTGGACGCAATGGCCAAATCCGCCGTCGAGGTCGATGGCCGCGGGCTGGGTGTGCGC
GGCACCAGCGCATCTTGTGACGAGTCTTCTTCGTCCTCGGACTCGAGACGCCGCCTCAGCCGCTTT
TTTGGGGGCGCGCGGGGAGGCGGCGGCGACGGCGACGGGGACGACACGTCCTCCATGGTTGGTGGA
CGTCGCGCCGCACCGCGTCCGCGCTCGGGGGTGGTTTCGCGCTGCTCCTCTTCCCGACTGGCCATT
TCCTTCTCCTATAGGCAGAAAAAGATCATGGAGTCAGTCGAGAAGGAGGACAGCCTAACCGCCCCC
TTTGAGTTCGCCACCACCGCCTCCACCGATGCCGCCAACGCGCCTACCACCTTCCCCGTCGAGGCA
CCCCCGCTTGAGGAGGAGGAAGTGATTATCGAGCAGGACCCAGGTTTTGTAAGCGAAGACGACGAG
GATCGCTCAGTACCAACAGAGGATAAAAAGCAAGACCAGGACGACGCAGAGGCAAACGAGGAACAA
GTCGGGCGGGGGGACCAAAGGCATGGCGACTACCTAGATGTGGGAGACGACGTGCTGTTGAAGCAT
CTGCAGCGCCAGTGCGCCATTATCTGCGACGCGTTGCAAGAGCGCAGCGATGTGCCCCTCGCCATA
GCGGATGTCAGCCTTGCCTACGAACGCCACCTGTTCTCACCGCGCGTACCCCCCAAACGCCAAGAA
AACGGCACATGCGAGCCCAACCCGCGCCTCAACTTCTACCCCGTATTTGCCGTGCCAGAGGTGCTT
GCCACCTATCACATCTTTTTCCAAAACTGCAAGATACCCCTATCCTGCCGTGCCAACCGCAGCCGA
GCGGACAAGCAGCTGGCCTTGCGGCAGGGCGCTGTCATACCTGATATCGCCTCGCTCGACGAAGTG
CCAAAAATCTTTGAGGGTCTTGGACGCGACGAGAAACGCGCGGCAAACGCTCTGCAACAAGAAAAC
AGCGAAAATGAAAGTCACTGTGGAGTGCTGGTGGAACTTGAGGGTGACAACGCGCGCCTAGCCGTG
CTGAAACGCAGCATCGAGGTCACCCACTTTGCCTACCCGGCACTTAACCTACCCCCCAAGGTTATG
AGCACAGTCATGAGCGAGCTGATCGTGCGCCGTGCACGACCCCTGGAGAGGGATGCAAACTTGCAA
GAACAAACCGAGGAGGGCCTACCCGCAGTTGGCGATGAGCAGCTGGCGCGCTGGCTTGAGACGCGC
GAGCCTGCCGACTTGGAGGAGCGACGCAAGCTAATGATGGCCGCAGTGCTTGTTACCGTGGAGCTT
GAGTGCATGCAGCGGTTCTTTGCTGACCCGGAGATGCAGCGCAAGCTAGAGGAAACGTTGCACTAC
ACCTTTCGCCAGGGCTACGTGCGCCAGGCCTGCAAAATTTCCAACGTGGAGCTCTGCAACCTGGTC
TCCTACCTTGGAATTTTGCACGAAAACCGCCTCGGGCAAAACGTGCTTCATTCCACGCTCAAGGGC
GAGGCGCGCCGCGACTACGTCCGCGACTGCGTTTACTTATTTCTGTGCTACACCTGGCAAACGGCC
ATGGGCGTGTGGCAGCAATGCCTGGAGGAGCGCAACCTAAAGGAGCTGCAGAAGCTGCTAAAGCAA
AACTTGAAGGACCTATGGACGGCCTTCAACGAGCGCTCCGTGGCCGCGCACCTGGCGGACATTATC
TTCCCCGAACGCCTGCTTAAAACCCTGCAACAGGGTCTGCCAGACTTCACCAGTCAAAGCATGTTG
CAAAACTTTAGGAACTTTATCCTAGAGCGTTCAGGAATTCTGCCCGCCACCTGCTGTGCGCTTCCT
AGCGACTTTGTGCCCATTAAGTACCGTGAATGCCCTCCGCCGCTTTGGGGTCACTGCTACCTTCTG
CAGCTAGCCAACTACCTTGCCTACCACTCCGACATCATGGAAGACGTGAGCGGTGACGGCCTACTG
GAGTGTCACTGTCGCTGCAACCTATGCACCCCGCACCGCTCCCTGGTCTGCAATTCGCAACTGCTT
AGCGAAAGTCAAATTATCGGTACCTTTGAGCTGCAGGGTCCCTCGCCTGACGAAAAGTCCGCGGCT
CCGGGGTTGAAACTCACTCCGGGGCTGTGGACGTCGGCTTACCTTCGCAAATTTGTACCTGAGGAC
TACCACGCCCACGAGATTAGGTTCTACGAAGACCAATCCCGCCCGCCAAATGCGGAGCTTACCGCC
TGCGTCATTACCCAGGGCCACATCCTTGGCCAATTGCAAGCCATCAACAAAGCCCGCCAAGAGTTT
CTGCTACGAAAGGGACGGGGGGTTTACCTGGACCCCCAGTCCGGCGAGGAGCTCAACCCAATCCCC
CCGCCGCCGCAGCCCTATCAGCAGCCGCGGGCCCTTGCTTCCCAGGATGGCACCCAAAAAGAAGCT
GCAGCTGCCGCCGCCGCCACCCACGGACGAGGAGGAATACTGGGACAGTCAGGCAGAGGAGGTTTT
GGACGAGGAGGAGGAGATGATGGAAGACTGGGACAGCCTAGACGAAGCTTCCGAGGCCGAAGAGGT
GTCAGACGAAACACCGTCACCCTCGGTCGCATTCCCCTCGCCGGCGCCCCAGAAATTGGCAACCGT
TCCCAGCATCGCTACAACCTCCGCTCCTCAGGCGCCGCCGGCACTGCCTGTTCGCCGACCCAACCG
TAGATGGGACACCACTGGAACCAGGGCCGGTAAGTCTAAGCAGCCGCCGCCGTTAGCCCAAGAGCA
ACAACAGCGCCAAGGCTACCGCTCGTGGCGCGGGCACAAGAACGCCATAGTTGCTTGCTTGCAAGA
CTGTGGGGGCAACATCTCCTTCGCCCGCCGCTTTCTTCTCTACCATCACGGCGTGGCCTTCCCCCG
TAACATCCTGCATTACTACCGTCATCTCTACAGCCCCTACTGCACCGGCGGCAGCGGCAGCGGCAG
CAACAGCAGCGGTCACACAGAAGCAAAGGCGACCGGATAGCAAGACTCTGACAAAGCCCAAGAAAT
CCACAGCGGCGGCAGCAGCAGGAGGAGGAGCGCTGCGTCTGGCGCCCAACGAACCCGTATCGACCC
GCGAGCTTAGAAATAGGATTTTTCCCACTCTGTATGCTATATTTCAACAAAGCAGGGGCCAAGAAC
AAGAGCTGAAAATAAAAAACAGGTCTCTGCGCTCCCTCACCCGCAGCTGCCTGTATCACAAAAGCG
AAGATCAGCTTCGGCGCACGCTGGAAGACGCGGAGGCTCTCTTCAGCAAATACTGCGCGCTGACTC
TTAAGGACTAGTTTCGCGCCCTTTCTCAAATTTAAGCGCGAAAACTACGTCATCTCCAGCGGCCAC
ACCCGGCGCCAGCACCTGTCGTCAGCGCCATTATGAGCAAGGAAATTCCCACGCCCTACATGTGGA
GTTACCAGCCACAAATGGGACTTGCGGCTGGAGCTGCCCAAGACTACTCAACCCGAATAAACTACA
TGAGCGCGGGACCCCACATGATATCCCGGGTCAACGGAATCCGCGCCCACCGAAACCGAATTCTCC
TCGAACAGGCGGCTATTACCACCACACCTCGTAATAACCTTAATCCCCGTAGTTGGCCCGCTGCCC
TGGTGTACCAGGAAAGTCCCGCTCCCACCACTGTGGTACTTCCCAGAGACGCCCAGGCCGAAGTTC
AGATGACTAACTCAGGGGCGCAGCTTGCGGGCGGCTTTCGTCACAGGGTGCGGTCGCCCGGGCGTT
TTAGGGCGGAGTAACTTGCATGTATTGGGAATTGTAGTTTTTTTAAAATGGGAAGTGACGTATCGT
GGGAAAACGGAAGTGAAGATTTGAGGAAGTTGTGGGTTTTTTGGCTTTCGTTTCTGGGCGTAGGTT
CGCGTGCGGTTTTCTGGGTGTTTTTTGTGGACTTTAACCGTTACGTCATTTTTTAGTCCTATATAT
ACTCGCTCTGTACTTGGCCCTTTTTACACTGTGACTGATTGAGCTGGTGCCGTGTCGAGTGGTGTT
TTTTAATAGGTTTTTTTACTGGTAAGGCTGACTGTTATGGCTGCCGCTGTGGAAGCGCTGTATGTT
GTTCTGGAGCGGGAGGGTGCTATTTTGCCTAGGCAGGAGGGTTTTTCAGGTGTTTATGTGTTTTTC
TCTCCTATTAATTTTGTTATACCTCCTATGGGGGCTGTAATGTTGTCTCTACGCCTGCGGGTATGT
ATTCCCCCGGGCTATTTCGGTCGCTTTTTAGCACTGACCGATGTTAACCAACCTGATGTGTTTACC
GAGTCTTACATTATGACTCCGGACATGACCGAGGAACTGTCGGTGGTGCTTTTTAATCACGGTGAC
CAGTTTTTTTACGGTCACGCCGGCATGGCCGTAGTCCGTCTTATGCTTATAAGGGTTGTTTTTCCT
GTTGTAAGACAGGCTTCTAATGTTTAAATGTTTTTTTTTTTGTTATTTTATTTTGTGTTTAATGCA
GGAACCCGCAGACATGTTTGAGAGAAAAATGGTGTCTTTTTCTGTGGTGGTTCCGGAACTTACCTG
CCTTTATCTGCATGAGCATGACTACGATGTGCTTGCTTTTTTGCGCGAGGCTTTGCCTGATTTTTT
GAGCAGCACCTTGCATTTTATATCGCCGCCCATGCAACAAGCTTACATAGGGGCTACGCTGGTTAG
CATAGCTCCGAGTATGCGTGTCATAATCAGTGTGGGTTCTTTTGTCATGGTTCCTGGCGGGGAAGT
GGCCGCGCTGGTCCGTGCAGACCTGCACGATTATGTTCAGCTGGCCCTGCGAAGGGACCTACGGGA
TCGCGGTATTTTTGTTAATGTTCCGCTTTTGAATCTTATACAGGTCTGTGAGGAACCTGAATTTTT
GCAATCATGATTCGCTGCTTGAGGCTGAAGGTGGAGGGCGCTCTGGAGCAGATTTTTACAATGGCC
GGACTTAATATTCGGGATTTGCTTAGAGACATATTGATAAGGTGGCGAGATGAAAATTATTTGGGC
ATGGTTGAAGGTGCTGGAATGTTTATAGAGGAGATTCACCCTGAAGGGTTTAGCCTTTACGTCCAC
TTGGACGTGAGGGCAGTTTGCCTTTTGGAAGCCATTGTGCAACATCTTACAAATGCCATTATCTGT
TCTTTGGCTGTAGAGTTTGACCACGCCACCGGAGGGGAGCGCGTTCACTTAATAGATCTTCATTTT
GAGGTTTTGGATAATCTTTTGGAATAAAAAAAAAAAAACATGGTTCTTCCAGCTCTTCCCGCTCCT
CCCGTGTGTGACTCGCAGAACGAATGTGTAGGTTGGCTGGGTGTGGCTTATTCTGCGGTGGTGGAT
GTTATCAGGGCAGCGGCGCATGAAGGAGTTTACATAGAACCCGAAGCCAGGGGGCGCCTGGATGCT
TTGAGAGAGTGGATATACTACAACTACTACACAGAGCGAGCTAAGCGACGAGACCGGAGACGCAGA
TCTGTTTGTCACGCCCGCACCTGGTTTTGCTTCAGGAAATATGACTACGTCCGGCGTTCCATTTGG
CATGACACTACGACCAACACGATCTCGGTTGTCTCGGCGCACTCCGTACAGTAGGGATCGCCTACC
TCCTTTTGAGACAGAGACCCGCGCTACCATACTGGAGGATCATCCGCTGCTGCCCGAATGTAACAC
TTTGACAATGCACAACGTGAGTTACGTGCGAGGTCTTCCCTGCAGTGTGGGATTTACGCTGATTCA
GGAATGGGTTGTTCCCTGGGATATGGTTCTGACGCGGGAGGAGCTTGTAATCCTGAGGAAGTGTAT
GCACGTGTGCCTGTGTTGTGCCAACATTGATATCATGACGAGCATGATGATCCATGGTTACGAGTC
CTGGGCTCTCCACTGTCATTGTTCCAGTCCCGGTTCCCTGCAGTGCATAGCCGGCGGGCAGGTTTT
GGCCAGCTGGTTTAGGATGGTGGTGGATGGCGCCATGTTTAATCAGAGGTTTATATGGTACCGGGA
GGTGGTGAATTACAACATGCCAAAAGAGGTAATGTTTATGTCCAGCGTGTTTATGAGGGGTCGCCA
CTTAATCTACCTGCGCTTGTGGTATGATGGCCACGTGGGTTCTGTGGTCCCCGCCATGAGCTTTGG
ATACAGCGCCTTGCACTGTGGGATTTTGAACAATATTGTGGTGCTGTGCTGCAGTTACTGTGCTGA
TTTAAGTGAGATCAGGGTGCGCTGCTGTGCCCGGAGGACAAGGCGTCTCATGCTGCGGGCGGTGCG
AATCATCGCTGAGGAGACCACTGCCATGTTGTATTCCTGCAGGACGGAGCGGCGGCGGCAGCAGTT
TATTCGCGCGCTGCTGCAGCACCACCGCCCTATCCTGATGCACGATTATGACTCTACCCCCATGTA
GGCGTGGACTTCCCCTTCGCCGCCCGTTGAGCAACCGCAAGTTGGACAGCAGCCTGTGGCTCAGCA
GCTGGACAGCGACATGAACTTAAGCGAGCTGCCCGGGGAGTTTATTAATATCACTGATGAGCGTTT
GGCTCGACAGGAAACCGTGTGGAATATAACACCTAAGAATATGTCTGTTACCCATGATATGATGCT
TTTTAAGGCCAGCCGGGGAGAAAGGACTGTGTACTCTGTGTGTTGGGAGGGAGGTGGCAGGTTGAA
TACTAGGGTTCTGTGAGTTTGATTAAGGTACGGTGATCAATATAAGCTATGTGGTGGTGGGGCTAT
ACTACTGAATGAAAAATGACTTGAAATTTTCTGCAATTGAAAAATAAACACGTTGAAACATAACAT
GCAACAGGTTCACGATTCTTTATTCCTGGGCAATGTAGGAGAAGGTGTAAGAGTTGGTAGCAAAAG
TTTCAGTGGTGTATTTTCCACTTTCCCAGGACCATGTAAAAGACATAGAGTAAGTGCTTACCTCGC
TAGTTTCTGTGGATTCACTAGAATCGATGTCGACGGGCACTCTTCCGTGGTCTGGTGGATAAATTC
GCAAGGGTATCATGGCGGACGACCGGGATTCGAACCCCGGATCCGGCCGTCCGCCGTGATCCATGC
GGTTACCGCCCGCGTGTCGAACCCAGGTGTGCGACGTCAGACAACGGGGGAGCGCTCCTTTTTCAT
ATGATCAGCTCACTCAAAGGCGGTAATACGGTTATCCACAGAATCAGGGGATAACGCAGGAAAGAA
CATGTGAGCAAAAGGCCAGCAAAAGGCCAGGAACCGTAAAAAGGCCGCGTTGCTGGCGTTTTTCCA
TAGGCTCCGCCCCCCTGACGAGCATCACAAAAATCGACGCTCAAGTCAGAGGTGGCGAAACCCGAC
AGGACTATAAAGATACCAGGCGTTTCCCCCTGGAAGCTCCCTCGTGCGCTCTCCTGTTCCGACCCT
GCCGCTTACCGGATACCTGTCCGCCTTTCTCCCTTCGGGAAGCGTGGCGCTTTCTCATAGCTCACG
CTGTAGGTATCTCAGTTCGGTGTAGGTCGTTCGCTCCAAGCTGGGCTGTGTGCACGAACCCCCCGT
TCAGCCCGACCGCTGCGCCTTATCCGGTAACTATCGTCTTGAGTCCAACCCGGTAAGACACGACTT
ATCGCCACTGGCAGCAGCCACTGGTAACAGGATTAGCAGAGCGAGGTATGTAGGCGGTGCTACAGA
GTTCTTGAAGTGGTGGCCTAACTACGGCTACACTAGAAGAACAGTATTTGGTATCTGCGCTCTGCT
GAAGCCAGTTACCTTCGGAAAAAGAGTTGGTAGCTCTTGATCCGGCAAACAAACCACCGCTGGTAG
CGGTGGTTTTTTTGTTTGCAAGCAGCAGATTACGCGCAGAAAAAAAGGATCTCAAGAAGATCCTTT
GATCTTTTCTACGGGGTCTGACGCTCAGTGGAACGAAAACTCACGTTAAGGGATTTTGGTCATGAG
ATTATCAAAAAGGATCTTCACCTAGATCCTTTTAAATTAAAAATGAAGTTTTAAATCAATCTAAAG
TATATATGAGTAAACTTGGTCTGACAGTTACCAATGCTTAATCAGTGAGGCACCTATCTCAGCGAT
CTGTCTATTTCGTTCATCCATAGTTGCCTGACTCCCCGTCGTGTAGATAACTACGATACGGGAGGG
CTTACCATCTGGCCCCAGTGCTGCAATGATACCGCGAGACCCACGCTCACCGGCTCCAGATTTATC
AGCAATAAACCAGCCAGCCGGAAGGGCCGAGCGCAGAAGTGGTCCTGCAACTTTATCCGCCTCCAT
CCAGTCTATTAATTGTTGCCGGGAAGCTAGAGTAAGTAGTTCGCCAGTTAATAGTTTGCGCAACGT
TGTTGCCATTGCTACAGGCATCGTGGTGTCACGCTCGTCGTTTGGTATGGCTTCATTCAGCTCCGG
TTCCCAACGATCAAGGCGAGTTACATGATCCCCCATGTTGTGCAAAAAAGCGGTTAGCTCCTTCGG
TCCTCCGATCGTTGTCAGAAGTAAGTTGGCCGCAGTGTTATCACTCATGGTTATGGCAGCACTGCA
TAATTCTCTTACTGTCATGCCATCCGTAAGATGCTTTTCTGTGACTGGTGAGTACTCAACCAAGTC
ATTCTGAGAATAGTGTATGCGGCGACCGAGTTGCTCTTGCCCGGCGTCAATACGGGATAATACCGC
GCCACATAGCAGAACTTTAAAAGTGCTCATCATTGGAAAACGTTCTTCGGGGCGAAAACTCTCAAG
GATCTTACCGCTGTTGAGATCCAGTTCGATGTAACCCACTCGTGCACCCAACTGATCTTCAGCATC
TTTTACTTTCACCAGCGTTTCTGGGTGAGCAAAAACAGGAAGGCAAAATGCCGCAAAAAAGGGAAT
AAGGGCGACACGGAAATGTTGAATACTCATACTCTTCCTTTTTCAATATTATTGAAGCATTTATCA
GGGTTATTGTCTCATGAGCGGATACATATTTGAATGTATTTAGAAAAATAAACAAATAGGGGTTCC
GCGCACATTTCCCCGAAAAGTGCCACCTAAATTGTAAGCGTTAATATTTTGTTAAAATTCGCGTTA
AATTTTTGTTAAATCAGCTCATTTTTTAACCAATAGGCCGAAATCGGCAAAATCCCTTATAAATCA
AAAGAATAGACCGAGATAGGGTTGAGTGTTGTTCCAGTTTGGAACAAGAGTCCACTATTAAAGAAC
GTGGACTCCAACGTCAAAGGGCGAAAAACCGTCTATCAGGGCGATGGCCCACTACGTGAACCATCA
CCCTAATCAAGTTTTTTGGGGTCGAGGTGCCGTAAAGCACTAAATCGGAACCCTAAAGGGAGCCCC
CGATTTAGAGCTTGACGGGGAAAGCCGGCGAACGTGGCGAGAAAGGAAGGGAAGAAAGCGAAAGGA
GCGGGCGCTAGGGCGCTGGCAAGTGTAGCGGTCACGCTGCGCGTAACCACCACACCCGCCGCGCTT
AATGCGCCGCTACAGGGCGCGATGGATCC
SEQ ID NO: 25 (STXC0030)
CCGCGGCCGCCAACTTTGTATAGAAAAGTTGTAGTTATTAATAGTAATCAATTACGGGGTCATTAG
TTCATAGCCCATATATGGAGTTCCGCGTTACATAACTTACGGTAAATGGCCCGCCTGGCTGACCGC
CCAACGACCCCCGCCCATTGACGTCAATAATGACGTATGTTCCCATAGTAACGCCAATAGGGACTT
TCCATTGACGTCAATGGGTGGAGTATTTACGGTAAACTGCCCACTTGGCAGTACATCAAGTGTATC
ATATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCATGG
TGATGCGGTTTTGGCAGTACATCAATGGGCGTGGATAGCGGTTTGACTCACGGGGATTTCCAAGTC
TCCACCCCATTGACGTCAATGGGAGTTTGTTTTGGCACCAAAATCAACGGGACTTTCCAAAATGTC
GTAACAACTCCGCCCCATTGACGCAAATGGGCGGTAGGCGTGTACGGTGGGAGGTCTATATAAGCA
GAGCTGGTTTAGTGAACCGTCAGATCCAAGTTTGTACAAAAAAGCAGGCTGCCACCATGGCCAGTC
GGGAAGAGGAGCAGCGCGAAACCACCCCCGAGCGCGGACGCGGTGCGGCGCGACGTCCACCAACCA
TGGAGGACGTGTCGTCCCCGTCGCCGTCGCCGCCGCCTCCCCGCGCGCCCCCAAAAAAGCGGCTGA
GGCGGCGTCTCGAGTCCGAGGACGAAGAAGACTCGTCACAAGATGCGCTGGTGCCGCGCACACCCA
GCCCGCGGCCATCGACCTCGACGGCGGATTTGGCCATTGCGTCCAAAAAGAAAAAGAAGCGCCCCT
CTCCCAAGCCCGAGCGCCCGCCATCCCCAGAGGTGATCGTGGACAGCGAGGAAGAAAGAGAAGATG
TGGCGCTACAAATGGTGGGTTTCAGCAACCCACCGGTGCTAATCAAGCACGGCAAGGGAGGTAAGC
GCACGGTGCGGCGGCTGAATGAAGACGACCCAGTGGCGCGGGGTATGCGGACGCAAGAGGAAAAGG
AAGAGTCCAGTGAAGCGGAAAGTGAAAGCACGGTGATAAACCCGCTGAGCCTGCCGATCGTGTCTG
CGTGGGAGAAGGGCATGGAGGCTGCGCGCGCGTTGATGGACAAGTACCACGTGGATAACGATCTAA
AGGCAAACTTCAAGCTACTGCCTGACCAAGTGGAAGCTCTGGCGGCCGTATGCAAGACCTGGCTAA
ACGAGGAGCACCGCGGGTTGCAGCTGACCTTCACCAGCAACAAGACCTTTGTGACGATGATGGGGC
GATTCCTGCAGGCGTACCTGCAGTCGTTTGCAGAGGTAACCTACAAGCACCACGAGCCCACGGGCT
GCGCGTTGTGGCTGCACCGCTGCGCTGAGATCGAAGGCGAGCTTAAGTGTCTACACGGGAGCATTA
TGATAAATAAGGAGCACGTGATTGAAATGGATGTGACGAGCGAAAACGGGCAGCGCGCGCTGAAGG
AGCAGTCTAGCAAGGCCAAGATCGTGAAGAACCGGTGGGGCCGAAATGTGGTGCAGATCTCCAACA
CCGACGCAAGGTGCTGCGTGCATGACGCGGCCTGTCCGGCCAATCAGTTTTCCGGCAAGTCTTGCG
GCATGTTCTTCTCTGAAGGCGCAAAGGCTCAGGTGGCTTTTAAGCAGATCAAGGCTTTCATGCAGG
CGCTGTATCCTAACGCCCAGACCGGGCACGGTCACCTTCTGATGCCACTACGGTGCGAGTGCAACT
CAAAGCCTGGGCATGCACCCTTTTTGGGAAGGCAGCTACCAAAGTTGACTCCGTTCGCCCTGAGCA
ACGCGGAGGACCTGGACGCGGATCTGATCTCCGACAAGAGCGTGCTGGCCAGCGTGCACCACCCGG
CGCTGATAGTGTTCCAGTGCTGCAACCCTGTGTATCGCAACTCGCGCGCGCAGGGCGGAGGCCCCA
ACTGCGACTTCAAGATATCGGCGCCCGACCTGCTAAACGCGTTGGTGATGGTGCGCAGCCTGTGGA
GTGAAAACTTCACCGAGCTGCCGCGGATGGTTGTGCCTGAGTTTAAGTGGAGCACTAAACACCAGT
ATCGCAACGTGTCCCTGCCAGTGGCGCATAGCGATGCGCGGCAGAACCCCTTTGATTTTTAAACCC
AGCTTTCTTGTACAAAGTGGGCCCCTCTCCCTCCCCCCCCCCTAACGTTACTGGCCGAAGCCGCTT
GGAATAAGGCCGGTGTGCGTTTGTCTATATGTTATTTTCCACCATATTGCCGTCTTTTGGCAATGT
GAGGGCCCGGAAACCTGGCCCTGTCTTCTTGACGAGCATTCCTAGGGGTCTTTCCCCTCTCGCCAA
AGGAATGCAAGGTCTGTTGAATGTCGTGAAGGAAGCAGTTCCTCTGGAAGCTTCTTGAAGACAAAC
AACGTCTGTAGCGACCCTTTGCAGGCAGCGGAACCCCCCACCTGGCGACAGGTGCCTCTGCGGCCA
AAAGCCACGTGTATAAGATACACCTGCAAAGGCGGCACAACCCCAGTGCCACGTTGTGAGTTGGAT
AGTTGTGGAAAGAGTCAAATGGCTCTCCTCAAGCGTATTCAACAAGGGGCTGAAGGATGCCCAGAA
GGTACCCCATTGTATGGGATCTGATCTGGGGCCTCGGTGCACATGCTTTACATGTGTTTAGTCGAG
GTTAAAAAAACGTCTAGGCCCCCCGAACCACGGGGACGTGGTTTTCCTTTGAAAAACACGATGATA
ATATGGCCACAACCATGACTACGTCCGGCGTTCCATTTGGCATGACACTACGACCAACACGATCTC
GGTTGTCTCGGCGCACTCCGTACAGTAGGGATCGCCTACCTCCTTTTGAGACAGAGACCCGCGCTA
CCATACTGGAGGATCATCCGCTGCTGCCCGAATGTAACACTTTGACAATGCACAACGTGAGTTACG
TGCGAGGTCTTCCCTGCAGTGTGGGATTTACGCTGATTCAGGAATGGGTTGTTCCCTGGGATATGG
TTCTGACGCGGGAGGAGCTTGTAATCCTGAGGAAGTGTATGCACGTGTGCCTGTGTTGTGCCAACA
TTGATATCATGACGAGCATGATGATCCATGGTTACGAGTCCTGGGCTCTCCACTGTCATTGTTCCA
GTCCCGGTTCCCTGCAGTGCATAGCCGGCGGGCAGGTTTTGGCCAGCTGGTTTAGGATGGTGGTGG
ATGGCGCCATGTTTAATCAGAGGTTTATATGGTACCGGGAGGTGGTGAATTACAACATGCCAAAAG
AGGTAATGTTTATGTCCAGCGTGTTTATGAGGGGTCGCCACTTAATCTACCTGCGCTTGTGGTATG
ATGGCCACGTGGGTTCTGTGGTCCCCGCCATGAGCTTTGGATACAGCGCCTTGCACTGTGGGATTT
TGAACAATATTGTGGTGCTGTGCTGCAGTTACTGTGCTGATTTAAGTGAGATCAGGGTGCGCTGCT
GTGCCCGGAGGACAAGGCGTCTCATGCTGCGGGCGGTGCGAATCATCGCTGAGGAGACCACTGCCA
TGTTGTATTCCTGCAGGACGGAGCGGCGGCGGCAGCAGTTTATTCGCGCGCTGCTGCAGCACCACC
GCCCTATCCTGATGCACGATTATGACTCTACCCCCATGTAGCAACTTTATTATACATAGTTGATGG
CCGGCCGCTTCGAGCAGACATGATAAGATACATTGATGAGTTTGGACAAACCACAACTAGAATGCA
GTGAAAAAAATGCTTTATTTGTGAAATTTGTGATGCTATTGCTTTATTTGTAACCATTATAAGCTG
CAATAAACAAGTTAACAACAACAATTGCATTCATTTTATGTTTCAGGTTCAGGGGGAGGTGTGGGA
GGTTTTTTAAAGCAAGTAAAACCTCTACAAATGTGGTAGCGGCCGCGGCGCTCTTCCGCTTCCTCG
CTCACTGACTCGCTGCGCTCGGTCGTTCGGCTGCGGCGAGCGGTATCAGCTCACTCAAAGGCGGTA
ATACGGTTATCCACAGAATCAGGGGATAACGCAGGAAAGAACATGTGAGCAAAAGGCCAGCAAAAG
GCCAGGAACCGTAAAAAGGCCGCGTTGCTGGCGTTTTTCCATAGGCTCCGCCCCCCTGACGAGCAT
CACAAAAATCGACGCTCAAGTCAGAGGTGGCGAAACCCGACAGGACTATAAAGATACCAGGCGTTT
CCCCCTGGAAGCTCCCTCGTGCGCTCTCCTGTTCCGACCCTGCCGCTTACCGGATACCTGTCCGCC
TTTCTCTCTTCGGGAAGCGTGGCGCTTTCTCATAGCTCACGCTGTAGGTATCTCAGTTCGGTGTAG
GTCGTTCGCTCCAAGCTGGGCTGTGTGCACGAACCCCCCGTTCAGCCCGACCGCTGCGCCTTATCC
GGTAACTATCGTCTTGAGTCCAACCCGGTAAGACACGACTTATCGCCACTGGCAGCAGCCACTGGT
AACAGGATTAGCAGAGCGAGGTATGTAGGCGGTGCTACAGAGTTCTTGAAGTGGTGGCCTAACTAC
GGCTACACTAGAAGAACAGTATTTGGTATCTGCGCTCTGCTGAAGCCAGTTACCTTCGGAAAAAGA
GTTGGTAGCTCTTGATCCGGCAAACAAACCACCGCTGGTAGCGGTGGTTTTTTTGTTTGCAAGCAG
CAGATTACGCGCAGAAAAAAAGGATCTCAAGAAGATCCTTTGATCTTTTCTACGGGGTCTGACGCT
CAGTGGAACGAAAACTCACGTTAAGGGATTTTGGTCATGAGATTATCAAAAAGGATCTTCACCTAG
ATCCTTTTAAATTAAAAATGAAGTTTTAAATCAATCTAAAGTATATATGAGTAAACTTGGTCTGAC
AGTTACCAATGCTTAATCAGTGAGGCACCTATCTCAGCGATCTGTCTATTTCGTTCATCCATAGTT
GCCTGACTCCCCGTCGTGTAGATAACTACGATACGGGAGGGCTTACCATCTGGCCCCAGTGCTGCA
ATGATACCGCGAGACCCACGCTCACCGGCTCCAGATTTATCAGCAATAAACCAGCCAGCCGGAAGG
GCCGAGCGCAGAAGTGGTCCTGCAACTTTATCCGCCTCCATCCAGTCTATTAATTGTTGCCGGGAA
GCTAGAGTAAGTAGTTCGCCAGTTAATAGTTTGCGCAACGTTGTTGCCATTGCTACAGGCATCGTG
GTGTCACGCTCGTCGTTTGGTATGGCTTCATTCAGCTCCGGTTCCCAACGATCAAGGCGAGTTACA
TGATCCCCCATGTTGTGCAAAAAAGCGGTTAGCTCCTTCGGTCCTCCGATCGTTGTCAGAAGTAAG
TTGGCCGCAGTGTTATCACTCATGGTTATGGCAGCACTGCATAATTCTCTTACTGTCATGCCATCC
GTAAGATGCTTTTCTGTGACTGGTGAGTACTCAACCAAGTCATTCTGAGAATAGTGTATGCGGCGA
CCGAGTTGCTCTTGCCCGGCGTCAATACGGGATAATACCGCGCCACATAGCAGAACTTTAAAAGTG
CTCATCATTGGAAAACGTTCTTCGGGGCGAAAACTCTCAAGGATCTTACCGCTGTTGAGATCCAGT
TCGATGTAACCCACTCGTGCACCCAACTGATCTTCAGCATCTTTTACTTTCACCAGCGTTTCTGGG
TGAGCAAAAACAGGAAGGCAAAATGCCGCAAAAAAGGGAATAAGGGCGACACGGAAATGTTGAATA
CTCATACTCTTCCTTTTTCAATATTATTGAAGCATTTATCAGGGTTATTGTCTCATGAGCGGATAC
ATATTTGAATGTATTTAGAAAAATAAACAAATAGGGGTTCCGCGCACATTTCCCCGAAAAGTGCCA
CCTGACGTCTAAGAAACCATTATTATCATGACATTAACCTATAAAAATAGGCGTATCACGAGGCCC
TTTCGTCGGCGCGCCTCGATGTAGGATGTTGCCCCTCCTGACGCGGTAGGAGAAGGGGAGGGTGCC
CTGCATGTCTGCCGCTGCTCTTGCTCTTGCCGCTGCTGAGGAGGGGGGCGCATCTGCCGCAGCACC
GGATGCATCTGGGAAAAGCAAAAAAGGGGCTCGTCCCTGTTTCCGGAGGAATTTGCAAGCGGGGTC
TTGCATGACGGGGAGGCAAACCCCCGTTCGCCGCAGTCCGGCCGGCCCGAGACTCGAACCGGGGGT
CCTGCGACTCAACCCTTGGAAAATAACCCTCCGGCTACAGGGAGCGAGCCACTTAATGCTTTCGCT
TTCCAGCCTAACCGCTTACGCCGCGCGCGGCCAGTGGCCAAAAAAGCTAGCGCAGCAGCCGCCGCG
CCTGGAAGGAAGCCAAAAGGAGCGCTCCCCCGTTGTCTGACGTCGCACACCTGGGTTCGACACGCG
GGCGGTAACCGCATGGATCACGGCGGACGGCCGGATCCGGGGTTCGAACCCCGGTCGTCCGCCATG
ATACCCTTGCGAATTTATCCACCAGACCACGGAAGAGTGCCCGCTTACAGGCTCTCCTTTTGCACG
GTCTAGAGCGTCAACGACTGCGCACGCCTCACCGGCCAGAGCGTCCCGACCATGGAGCACTTTTTG
CCGCTGCGCAACATCTGGAACCGCGTCCGCGACTTTCCGCGCGCCTCCACCACCGCCGCCGGCATC
ACCTGGATGTCCAGGTACATCTACGGATTACGGGCGCG
SEQ ID NO: 26 (STXC0031)
CCGCGGCCGCCAACTTTGTATAGAAAAGTTGTAGTTATTAATAGTAATCAATTACGGGGTCATTAG
TTCATAGCCCATATATGGAGTTCCGCGTTACATAACTTACGGTAAATGGCCCGCCTGGCTGACCGC
CCAACGACCCCCGCCCATTGACGTCAATAATGACGTATGTTCCCATAGTAACGCCAATAGGGACTT
TCCATTGACGTCAATGGGTGGAGTATTTACGGTAAACTGCCCACTTGGCAGTACATCAAGTGTATC
ATATGCCAAGTACGCCCCCTATTGACGTCAATGACGGTAAATGGCCCGCCTGGCATTATGCCCAGT
ACATGACCTTATGGGACTTTCCTACTTGGCAGTACATCTACGTATTAGTCATCGCTATTACCATGG
TGATGCGGTTTTGGCAGTACATCAATGGGCGTGGATAGCGGTTTGACTCACGGGGATTTCCAAGTC
TCCACCCCATTGACGTCAATGGGAGTTTGTTTTGGCACCAAAATCAACGGGACTTTCCAAAATGTC
GTAACAACTCCGCCCCATTGACGCAAATGGGCGGTAGGCGTGTACGGTGGGAGGTCTATATAAGCA
GAGCTGGTTTAGTGAACCGTCAGATCCAAGTTTGTACAAAAAAGCAGGCTGCCACCATGACTACGT
CCGGCGTTCCATTTGGCATGACACTACGACCAACACGATCTCGGTTGTCTCGGCGCACTCCGTACA
GTAGGGATCGCCTACCTCCTTTTGAGACAGAGACCCGCGCTACCATACTGGAGGATCATCCGCTGC
TGCCCGAATGTAACACTTTGACAATGCACAACGTGAGTTACGTGCGAGGTCTTCCCTGCAGTGTGG
GATTTACGCTGATTCAGGAATGGGTTGTTCCCTGGGATATGGTTCTGACGCGGGAGGAGCTTGTAA
TCCTGAGGAAGTGTATGCACGTGTGCCTGTGTTGTGCCAACATTGATATCATGACGAGCATGATGA
TCCATGGTTACGAGTCCTGGGCTCTCCACTGTCATTGTTCCAGTCCCGGTTCCCTGCAGTGCATAG
CCGGCGGGCAGGTTTTGGCCAGCTGGTTTAGGATGGTGGTGGATGGCGCCATGTTTAATCAGAGGT
TTATATGGTACCGGGAGGTGGTGAATTACAACATGCCAAAAGAGGTAATGTTTATGTCCAGCGTGT
TTATGAGGGGTCGCCACTTAATCTACCTGCGCTTGTGGTATGATGGCCACGTGGGTTCTGTGGTCC
CCGCCATGAGCTTTGGATACAGCGCCTTGCACTGTGGGATTTTGAACAATATTGTGGTGCTGTGCT
GCAGTTACTGTGCTGATTTAAGTGAGATCAGGGTGCGCTGCTGTGCCCGGAGGACAAGGCGTCTCA
TGCTGCGGGCGGTGCGAATCATCGCTGAGGAGACCACTGCCATGTTGTATTCCTGCAGGACGGAGC
GGCGGCGGCAGCAGTTTATTCGCGCGCTGCTGCAGCACCACCGCCCTATCCTGATGCACGATTATG
ACTCTACCCCCATGTAGACCCAGCTTTCTTGTACAAAGTGGGCCCCTCTCCCTCCCCCCCCCCTAA
CGTTACTGGCCGAAGCCGCTTGGAATAAGGCCGGTGTGCGTTTGTCTATATGTTATTTTCCACCAT
ATTGCCGTCTTTTGGCAATGTGAGGGCCCGGAAACCTGGCCCTGTCTTCTTGACGAGCATTCCTAG
GGGTCTTTCCCCTCTCGCCAAAGGAATGCAAGGTCTGTTGAATGTCGTGAAGGAAGCAGTTCCTCT
GGAAGCTTCTTGAAGACAAACAACGTCTGTAGCGACCCTTTGCAGGCAGCGGAACCCCCCACCTGG
CGACAGGTGCCTCTGCGGCCAAAAGCCACGTGTATAAGATACACCTGCAAAGGCGGCACAACCCCA
GTGCCACGTTGTGAGTTGGATAGTTGTGGAAAGAGTCAAATGGCTCTCCTCAAGCGTATTCAACAA
GGGGCTGAAGGATGCCCAGAAGGTACCCCATTGTATGGGATCTGATCTGGGGCCTCGGTGCACATG
CTTTACATGTGTTTAGTCGAGGTTAAAAAAACGTCTAGGCCCCCCGAACCACGGGGACGTGGTTTT
CCTTTGAAAAACACGATGATAATATGGCCACAACCATGGCCAGTCGGGAAGAGGAGCAGCGCGAAA
CCACCCCCGAGCGCGGACGCGGTGCGGCGCGACGTCCACCAACCATGGAGGACGTGTCGTCCCCGT
CGCCGTCGCCGCCGCCTCCCCGCGCGCCCCCAAAAAAGCGGCTGAGGCGGCGTCTCGAGTCCGAGG
ACGAAGAAGACTCGTCACAAGATGCGCTGGTGCCGCGCACACCCAGCCCGCGGCCATCGACCTCGA
CGGCGGATTTGGCCATTGCGTCCAAAAAGAAAAAGAAGCGCCCCTCTCCCAAGCCCGAGCGCCCGC
CATCCCCAGAGGTGATCGTGGACAGCGAGGAAGAAAGAGAAGATGTGGCGCTACAAATGGTGGGTT
TCAGCAACCCACCGGTGCTAATCAAGCACGGCAAGGGAGGTAAGCGCACGGTGCGGCGGCTGAATG
AAGACGACCCAGTGGCGCGGGGTATGCGGACGCAAGAGGAAAAGGAAGAGTCCAGTGAAGCGGAAA
GTGAAAGCACGGTGATAAACCCGCTGAGCCTGCCGATCGTGTCTGCGTGGGAGAAGGGCATGGAGG
CTGCGCGCGCGTTGATGGACAAGTACCACGTGGATAACGATCTAAAGGCAAACTTCAAGCTACTGC
CTGACCAAGTGGAAGCTCTGGCGGCCGTATGCAAGACCTGGCTAAACGAGGAGCACCGCGGGTTGC
AGCTGACCTTCACCAGCAACAAGACCTTTGTGACGATGATGGGGCGATTCCTGCAGGCGTACCTGC
AGTCGTTTGCAGAGGTAACCTACAAGCACCACGAGCCCACGGGCTGCGCGTTGTGGCTGCACCGCT
GCGCTGAGATCGAAGGCGAGCTTAAGTGTCTACACGGGAGCATTATGATAAATAAGGAGCACGTGA
TTGAAATGGATGTGACGAGCGAAAACGGGCAGCGCGCGCTGAAGGAGCAGTCTAGCAAGGCCAAGA
TCGTGAAGAACCGGTGGGGCCGAAATGTGGTGCAGATCTCCAACACCGACGCAAGGTGCTGCGTGC
ATGACGCGGCCTGTCCGGCCAATCAGTTTTCCGGCAAGTCTTGCGGCATGTTCTTCTCTGAAGGCG
CAAAGGCTCAGGTGGCTTTTAAGCAGATCAAGGCTTTCATGCAGGCGCTGTATCCTAACGCCCAGA
CCGGGCACGGTCACCTTCTGATGCCACTACGGTGCGAGTGCAACTCAAAGCCTGGGCATGCACCCT
TTTTGGGAAGGCAGCTACCAAAGTTGACTCCGTTCGCCCTGAGCAACGCGGAGGACCTGGACGCGG
ATCTGATCTCCGACAAGAGCGTGCTGGCCAGCGTGCACCACCCGGCGCTGATAGTGTTCCAGTGCT
GCAACCCTGTGTATCGCAACTCGCGCGCGCAGGGCGGAGGCCCCAACTGCGACTTCAAGATATCGG
CGCCCGACCTGCTAAACGCGTTGGTGATGGTGCGCAGCCTGTGGAGTGAAAACTTCACCGAGCTGC
CGCGGATGGTTGTGCCTGAGTTTAAGTGGAGCACTAAACACCAGTATCGCAACGTGTCCCTGCCAG
TGGCGCATAGCGATGCGCGGCAGAACCCCTTTGATTTTTAACAACTTTATTATACATAGTTGATGG
CCGGCCGCTTCGAGCAGACATGATAAGATACATTGATGAGTTTGGACAAACCACAACTAGAATGCA
GTGAAAAAAATGCTTTATTTGTGAAATTTGTGATGCTATTGCTTTATTTGTAACCATTATAAGCTG
CAATAAACAAGTTAACAACAACAATTGCATTCATTTTATGTTTCAGGTTCAGGGGGAGGTGTGGGA
GGTTTTTTAAAGCAAGTAAAACCTCTACAAATGTGGTAGCGGCCGCGGCGCTCTTCCGCTTCCTCG
CTCACTGACTCGCTGCGCTCGGTCGTTCGGCTGCGGCGAGCGGTATCAGCTCACTCAAAGGCGGTA
ATACGGTTATCCACAGAATCAGGGGATAACGCAGGAAAGAACATGTGAGCAAAAGGCCAGCAAAAG
GCCAGGAACCGTAAAAAGGCCGCGTTGCTGGCGTTTTTCCATAGGCTCCGCCCCCCTGACGAGCAT
CACAAAAATCGACGCTCAAGTCAGAGGTGGCGAAACCCGACAGGACTATAAAGATACCAGGCGTTT
CCCCCTGGAAGCTCCCTCGTGCGCTCTCCTGTTCCGACCCTGCCGCTTACCGGATACCTGTCCGCC
TTTCTCTCTTCGGGAAGCGTGGCGCTTTCTCATAGCTCACGCTGTAGGTATCTCAGTTCGGTGTAG
GTCGTTCGCTCCAAGCTGGGCTGTGTGCACGAACCCCCCGTTCAGCCCGACCGCTGCGCCTTATCC
GGTAACTATCGTCTTGAGTCCAACCCGGTAAGACACGACTTATCGCCACTGGCAGCAGCCACTGGT
AACAGGATTAGCAGAGCGAGGTATGTAGGCGGTGCTACAGAGTTCTTGAAGTGGTGGCCTAACTAC
GGCTACACTAGAAGAACAGTATTTGGTATCTGCGCTCTGCTGAAGCCAGTTACCTTCGGAAAAAGA
GTTGGTAGCTCTTGATCCGGCAAACAAACCACCGCTGGTAGCGGTGGTTTTTTTGTTTGCAAGCAG
CAGATTACGCGCAGAAAAAAAGGATCTCAAGAAGATCCTTTGATCTTTTCTACGGGGTCTGACGCT
CAGTGGAACGAAAACTCACGTTAAGGGATTTTGGTCATGAGATTATCAAAAAGGATCTTCACCTAG
ATCCTTTTAAATTAAAAATGAAGTTTTAAATCAATCTAAAGTATATATGAGTAAACTTGGTCTGAC
AGTTACCAATGCTTAATCAGTGAGGCACCTATCTCAGCGATCTGTCTATTTCGTTCATCCATAGTT
GCCTGACTCCCCGTCGTGTAGATAACTACGATACGGGAGGGCTTACCATCTGGCCCCAGTGCTGCA
ATGATACCGCGAGACCCACGCTCACCGGCTCCAGATTTATCAGCAATAAACCAGCCAGCCGGAAGG
GCCGAGCGCAGAAGTGGTCCTGCAACTTTATCCGCCTCCATCCAGTCTATTAATTGTTGCCGGGAA
GCTAGAGTAAGTAGTTCGCCAGTTAATAGTTTGCGCAACGTTGTTGCCATTGCTACAGGCATCGTG
GTGTCACGCTCGTCGTTTGGTATGGCTTCATTCAGCTCCGGTTCCCAACGATCAAGGCGAGTTACA
TGATCCCCCATGTTGTGCAAAAAAGCGGTTAGCTCCTTCGGTCCTCCGATCGTTGTCAGAAGTAAG
TTGGCCGCAGTGTTATCACTCATGGTTATGGCAGCACTGCATAATTCTCTTACTGTCATGCCATCC
GTAAGATGCTTTTCTGTGACTGGTGAGTACTCAACCAAGTCATTCTGAGAATAGTGTATGCGGCGA
CCGAGTTGCTCTTGCCCGGCGTCAATACGGGATAATACCGCGCCACATAGCAGAACTTTAAAAGTG
CTCATCATTGGAAAACGTTCTTCGGGGCGAAAACTCTCAAGGATCTTACCGCTGTTGAGATCCAGT
TCGATGTAACCCACTCGTGCACCCAACTGATCTTCAGCATCTTTTACTTTCACCAGCGTTTCTGGG
TGAGCAAAAACAGGAAGGCAAAATGCCGCAAAAAAGGGAATAAGGGCGACACGGAAATGTTGAATA
CTCATACTCTTCCTTTTTCAATATTATTGAAGCATTTATCAGGGTTATTGTCTCATGAGCGGATAC
ATATTTGAATGTATTTAGAAAAATAAACAAATAGGGGTTCCGCGCACATTTCCCCGAAAAGTGCCA
CCTGACGTCTAAGAAACCATTATTATCATGACATTAACCTATAAAAATAGGCGTATCACGAGGCCC
TTTCGTCGGCGCGCCTCGATGTAGGATGTTGCCCCTCCTGACGCGGTAGGAGAAGGGGAGGGTGCC
CTGCATGTCTGCCGCTGCTCTTGCTCTTGCCGCTGCTGAGGAGGGGGGCGCATCTGCCGCAGCACC
GGATGCATCTGGGAAAAGCAAAAAAGGGGCTCGTCCCTGTTTCCGGAGGAATTTGCAAGCGGGGTC
TTGCATGACGGGGAGGCAAACCCCCGTTCGCCGCAGTCCGGCCGGCCCGAGACTCGAACCGGGGGT
CCTGCGACTCAACCCTTGGAAAATAACCCTCCGGCTACAGGGAGCGAGCCACTTAATGCTTTCGCT
TTCCAGCCTAACCGCTTACGCCGCGCGCGGCCAGTGGCCAAAAAAGCTAGCGCAGCAGCCGCCGCG
CCTGGAAGGAAGCCAAAAGGAGCGCTCCCCCGTTGTCTGACGTCGCACACCTGGGTTCGACACGCG
GGCGGTAACCGCATGGATCACGGCGGACGGCCGGATCCGGGGTTCGAACCCCGGTCGTCCGCCATG
ATACCCTTGCGAATTTATCCACCAGACCACGGAAGAGTGCCCGCTTACAGGCTCTCCTTTTGCACG
GTCTAGAGCGTCAACGACTGCGCACGCCTCACCGGCCAGAGCGTCCCGACCATGGAGCACTTTTTG
CCGCTGCGCAACATCTGGAACCGCGTCCGCGACTTTCCGCGCGCCTCCACCACCGCCGCCGGCATC
ACCTGGATGTCCAGGTACATCTACGGATTACGGGCGCG
SEQ ID NO: 27 (STXC00124)
TGGTATGGCTTTTTCCCCGTATCCCCCCAGGTGTCTGCAGGCTCAAAGAGCAGCGAGAAGCGTTCA
GAGGAAAGCGATCCCGTGCCACCTTCCCCGTGCCCGGGCTGTCCCCGCACGCTGCCGGCTCGGGGA
TGCGGGGGGAGCGCCGGACCGGAGCGGAGCCCCGGGCGGCTCGCTGCTGCCCCCTAGCGGGGGAGG
GACGTAATTACATCCCTGGGGGCTTTGGGGGGGGGCTGTCCCTGATATCTATAACAAGAAAATATA
TATATAATAAGTTATCACGTAAGTAGAACATGAAATAACAATATAATTATCGTATGAGTTAAATCT
TAAAAGTCACGTAAAAGATAATCATGCGTCATTTTGACTCACGCGGTCGTTATAGTTCAAAATCAG
TGACACTTACCGCATTGACAAGCACGCCTCACGGGAGCTCCAAGCGGCGACTGAGATGTCCTAAAT
GCACAGCGACGGATTCGCGCTATTTAGAAAGAGAGAGCAATATTTCAAGAATGCATGCGTCAATTT
TACGCAGACTATCTTTCTAGGGTTAATCTAGCTGCATCAGGATCATATCGTCGGGTCTTTTTTCCG
GCTCAGTCATCGCCCAAGCTGGCGCTATCTGGGCATCGGGGAGGAAGAAGCCCGTGCCTTTTCCCG
CGAGGTTGAAGCGGCATGGAAAGAGTTTGCCGAGGATGACTGCTGCTGCATTGACGTTGAGCGAAA
ACGCACGTTTACCATGATGATTCGGGAAGGTGTGGCCATGCACGCCTTTAACGGTGAACTGTTCGT
TCAGGCCACCTGGGATACCAGTTCGTCGCGGCTTTTCCGGACACAGTTCCGGATGGTCAGCCCGAA
GCGCATCAGCAACCCGAACAATACCGGCGACAGCCGGAACTGCCGTGCCGGTGTGCAGATTAATGA
CAGCGGTGCGGCGCTGGGATATTACGTCAGCGAGGACGGGTATCCTGGCTGGATGCCGCAGAAATG
GACATGGATACCCCGTGAGTTACCCGGCGGGCGCGCTTGGCGTAATCATGGTCATAGCTGTTTCCT
GTGTGAAATTGTTATCCGCTCACAATTCCACACAACATACGAGCCGGAAGCATAAAGTGTAAAGCC
TGGGGTGCCTAATGAGTGAGCTAACTCACATTAATTGCGTTGCGCTCACTGCCCGCTTTCCAGTCG
GGAAACCTGTCGTGCCAGCTGCATTAATGAATCGGCCAACGCGCGGGGAGAGGCGGTTTGCGTATT
GGGCGCTCTTCCGCTTCCTCGCTCACTGACTCGCTGCGCTCGGTCGTTCGGCTGCGGCGAGCGGTA
TCAGCTCACTCAAAGGCGGTAATACGGTTATCCACAGAATCAGGGGATAACGCAGGAAAGAACATG
TGAGCAAAAGGCCAGCAAAAGGCCAGGAACCGTAAAAAGGCCGCGTTGCTGGCGTTTTTCCATAGG
CTCCGCCCCCCTGACGAGCATCACAAAAATCGACGCTCAAGTCAGAGGTGGCGAAACCCGACAGGA
CTATAAAGATACCAGGCGTTTCCCCCTGGAAGCTCCCTCGTGCGCTCTCCTGTTCCGACCCTGCCG
CTTACCGGATACCTGTCCGCCTTTCTCCCTTCGGGAAGCGTGGCGCTTTCTCATAGCTCACGCTGT
AGGTATCTCAGTTCGGTGTAGGTCGTTCGCTCCAAGCTGGGCTGTGTGCACGAACCCCCCGTTCAG
CCCGACCGCTGCGCCTTATCCGGTAACTATCGTCTTGAGTCCAACCCGGTAAGACACGACTTATCG
CCACTGGCAGCAGCCACTGGTAACAGGATTAGCAGAGCGAGGTATGTAGGCGGTGCTACAGAGTTC
TTGAAGTGGTGGCCTAACTACGGCTACACTAGAAGGACAGTATTTGGTATCTGCGCTCTGCTGAAG
CCAGTTACCTTCGGAAAAAGAGTTGGTAGCTCTTGATCCGGCAAACAAACCACCGCTGGTAGCGGT
GGTTTTTTTGTTTGCAAGCAGCAGATTACGCGCAGAAAAAAAGGATCTCAAGAAGATCCTTTGATC
TTTTCTACGGGGTCTGACGCTCAGTGGAACGAAAACTCACGTTAAGGGATTTTGGTCATGAGATTA
TCAAAAAGGATCTTCACCTAGATCCTTTTAAATTAAAAATGAAGTTTTAAATCAATCTAAAGTATA
TATGAGTAAACTTGGTCTGACAGTTACCAATGCTTAATCAGTGAGGCACCTATCTCAGCGATCTGT
CTATTTCGTTCATCCATAGTTGCCTGACTCCCCGTCGTGTAGATAACTACGATACGGGAGGGCTTA
CCATCTGGCCCCAGTGCTGCAATGATACCGCGAGACCCACGCTCACCGGCTCCAGATTTATCAGCA
ATAAACCAGCCAGCCGGAAGGGCCGAGCGCAGAAGTGGTCCTGCAACTTTATCCGCCTCCATCCAG
TCTATTAATTGTTGCCGGGAAGCTAGAGTAAGTAGTTCGCCAGTTAATAGTTTGCGCAACGTTGTT
GCCATTGCTACAGGCATCGTGGTGTCACGCTCGTCGTTTGGTATGGCTTCATTCAGCTCCGGTTCC
CAACGATCAAGGCGAGTTACATGATCCCCCATGTTGTGCAAAAAAGCGGTTAGCTCCTTCGGTCCT
CCGATCGTTGTCAGAAGTAAGTTGGCCGCAGTGTTATCACTCATGGTTATGGCAGCACTGCATAAT
TCTCTTACTGTCATGCCATCCGTAAGATGCTTTTCTGTGACTGGTGAGTACTCAACCAAGTCATTC
TGAGAATAGTGTATGCGGCGACCGAGTTGCTCTTGCCCGGCGTCAATACGGGATAATACCGCGCCA
CATAGCAGAACTTTAAAAGTGCTCATCATTGGAAAACGTTCTTCGGGGCGAAAACTCTCAAGGATC
TTACCGCTGTTGAGATCCAGTTCGATGTAACCCACTCGTGCACCCAACTGATCTTCAGCATCTTTT
ACTTTCACCAGCGTTTCTGGGTGAGCAAAAACAGGAAGGCAAAATGCCGCAAAAAAGGGAATAAGG
GCGACACGGAAATGTTGAATACTCATACTCTTCCTTTTTCAATATTATTGAAGCATTTATCAGGGT
TATTGTCTCATGAGCGGATACATATTTGAATGTATTTAGAAAAATAAACAAATAGGGGTTCCGCGC
ACATTTCCCCGAAAAGTGCCACCTAAATTGTAAGCGTTAATATTTTGTTAAAATTCGCGTTAAATT
TTTGTTAAATCAGCTCATTTTTTAACCAATAGGCCGAAATCGGCAAAATCCCTTATAAATCAAAAG
AATAGACCGAGATAGGGTTGAGTGTTGTTCCAGTTTGGAACAAGAGTCCACTATTAAAGAACGTGG
ACTCCAACGTCAAAGGGCGAAAAACCGTCTATCAGGGCGATGGCCCACTACGTGAACCATCACCCT
AATCAAGTTTTTTGGGGTCGAGGTGCCGTAAAGCACTAAATCGGAACCCTAAAGGGAGCCCCCGAT
TTAGAGCTTGACGGGGAAAGCCGGCGAACGTGGCGAGAAAGGAAGGGAAGAAAGCGAAAGGAGCGG
GCGCTAGGGCGCTGGCAAGTGTAGCGGTCACGCTGCGCGTAACCACCACACCCGCCGCGCTTAATG
CGCCGCTACAGGGCGCGTCCCATTCGCCATTCAGGCTGCGCAACTGTTGGGAAGGGCGATCGGTGC
GGGCCTCTTCGCTATTACGCCAGCTGGCGAAAGGGGGATGTGCTGCAAGGCGATTAAGTTGGGTAA
CGCCAGGGTTTTCCCAGTCACGACGTTGTAAAACGACGGCCAGTGAGCGCGCCTCGTTCATTCACG
TTTTTGAACCCGTGGAGGACGGGCAGACTCGCGGTGCAAATGTGTTTTACAGCGTGATGGAGCAGA
TGAAGATGCTCGACACGCTGCAGAACACGCAGCTAGATTAACCCTAGAAAGATAATCATATTGTGA
CGTACGTTAAAGATAATCATGTGTAAAATTGACGCATGTGTTTTATCGGTCTGTATATCGAGGTTT
ATTTATTAATTTGAATAGATATTAAGTTTTATTATATTTACACTTACATACTAATAATAAATTCAA
CAAACAATTTATTTATGTTTATTTATTTATTAAAAAAAACAAAAACTCAAAATTTCTTCTATAAAG
TAACAAAACTTTTATGAGGGACAGCCCCCCCCCAAAGCCCCCAGGGATGTAATTACGTCCCTCCCC
CGCTAGGGGGCAGCAGCGAGCCGCCCGGGGCTCCGCTCCGGTCCGGCGCTCCCCCCGCATCCCCGA
GCCGGCAGCGTGCGGGGACAGCCCGGGCACGGGGAAGGTGGCACGGGATCGCTTTCCTCTGAACGC
TTCTCGCTGCTCTTTGAGCCTGCAGACACCTGGGGGGATACGGGGAAAAGGCCTCCACGGCCACTA
GTCCATAGAGCCCACCGCATCCCCAGCATGCCTGCTATTGTCTTCCCAATCCTCCCCCTTGCTGTC
CTGCCCCACCCCACCCCCTAGAATAGAATGACACCTACTCAGACAATGCGATGCAATTTCCTCATT
TTATTAGGAAAGGACAGTGGGAGTGGCACCTTCCAGGGTCAAGGAAGGCACGGGGGAGGGGCAAAC
AACAGATGGCTGGCAACTAGAAGGCACAGCTACATGGGGGTAGAGTCATAATCGTGCATCAGGATA
GGGCGGTGGTGCTGCAGCAGCGCGCGAATAAACTGCTGCCGCCGCCGCTCCGTCCTGCAGGAATAC
AACATGGCAGTGGTCTCCTCAGCGATGATTCGCACCGCCCGCAGCATGAGACGCCTTGTCCTCCGG
GCACAGCAGCGCACCCTGATCTCACTTAAATCAGCACAGTAACTGCAGCACAGCACCACAATATTG
TTCAAAATCCCACAGTGCAAGGCGCTGTATCCAAAGCTCATGGCGGGGACCACAGAACCCACGTGG
CCATCATACCACAAGCGCAGGTAGATTAAGTGGCGACCCCTCATAAACACGCTGGACATAAACATT
ACCTCTTTTGGCATGTTGTAATTCACCACCTCCCGGTACCATATAAACCTCTGATTAAACATGGCG
CCATCCACCACCATCCTAAACCAGCTGGCCAAAACCTGCCCGCCGGCTATGCACTGCAGGGAACCG
GGACTGGAACAATGACAGTGGAGAGCCCAGGACTCGTAACCATGGATCATCATGCTCGTCATGATA
TCAATGTTGGCACAACACAGGCACACGTGCATACACTTCCTCAGGATTACAAGCTCCTCCCGCGTC
AGAACCATATCCCAGGGAACAACCCATTCCTGAATCAGCGTAAATCCCACACTGCAGGGAAGACCT
CGCACGTAACTCACGTTGTGCATTGTCAAAGTGTTACATTCGGGCAGCAGCGGATGATCCTCCAGT
ATGGTAGCGCGGGTCTCTGTCTCAAAAGGAGGTAGGCGATCCCTACTGTACGGAGTGCGCCGAGAC
AACCGAGATCGTGTTGGTCGTAGTGTCATGCCAAATGGAACGCCGGACGTAGTCATGGTTGTGGCC
ATATTATCATCGTGTTTTTCAAAGGAAAACCACGTCCCCGTGGTTCGGGGGGCCTAGACGTTTTTT
TAACCTCGACTAAACACATGTAAAGCATGTGCACCGAGGCCCCAGATCAGATCCCATACAATGGGG
TACCTTCTGGGCATCCTTCAGCCCCTTGTTGAATACGCTTGAGGAGAGCCATTTGACTCTTTCCAC
AACTATCCAACTCACAACGTGGCACTGGGGTTGTGCCGCCTTTGCAGGTGTATCTTATACACGTGG
CTTTTGGCCGCAGAGGCACCTGTCGCCAGGTGGGGGGTTCCGCTGCCTGCAAAGGGTCGCTACAGA
CGTTGTTTGTCTTCAAGAAGCTTCCAGAGGAACTGCTTCCTTCACGACATTCAACAGACCTTGCAT
TCCTTTGGCGAGAGGGGAAAGACCCCTAGGAATGCTCGTCAAGAAGACAGGGCCAGGTTTCCGGGC
CCTCACATTGCCAAAAGACGGCAATATGGTGGAAAATAACATATAGACAAACGCACACCGGCCTTA
TTCCAAGCGGCTTCGGCCAGTAACGTTAGGGGGGGGGGAGGGAGAGGGGCTTAAAAATCAAAGGGG
TTCTGCCGCGCATCACTATGCGCCACTGGCAGGGACACGTTGCGATACTGGTGTTTAGTGCTCCAC
TTAAACTCAGGCACAACCATCCGCGGCAGCTCGGTGAAGTTTTCACTCCACAGGCTGCGCACCATC
ACCAACGCGTTTAGCAGGTCGGGCGCCGATATCTTGAAGTCGCAGTTGGGGCCTCCGCCCTGCGCG
CGCGAGTTGCGATACACAGGGTTGCAGCACTGGAACACTATCAGCGCCGGGTGGTGCACGCTGGCC
AGCACGCTCTTGTCGGAGATCAGATCCGCGTCCAGGTCCTCCGCGTTGCTCAGGGCGAACGGAGTC
AACTTTGGTAGCTGCCTTCCCAAAAAGGGTGCATGCCCAGGCTTTGAGTTGCACTCGCACCGTAGT
GGCATCAGAAGGTGACCGTGCCCGGTCTGGGCGTTAGGATACAGCGCCTGCATGAAAGCCTTGATC
TGCTTAAAAGCCACCTGAGCCTTTGCGCCTTCAGAGAAGAACATGCCGCAAGACTTGCCGGAAAAC
TGATTGGCCGGACAGGCCGCGTCATGCACGCAGCACCTTGCGTCGGTGTTGGAGATCTGCACCACA
TTTCGGCCCCACCGGTTCTTCACGATCTTGGCCTTGCTAGACTGCTCCTTCAGCGCGCGCTGCCCG
TTTTCGCTCGTCACATCCATTTCAATCACGTGCTCCTTATTTATCATAATGCTCCCGTGTAGACAC
TTAAGCTCGCCTTCGATCTCAGCGCAGCGGTGCAGCCACAACGCGCAGCCCGTGGGCTCGTGGTGC
TTGTAGGTTACCTCTGCAAACGACTGCAGGTACGCCTGCAGGAATCGCCCCATCATCGTCACAAAG
GTCTTGTTGCTGGTGAAGGTCAGCTGCAACCCGCGGTGCTCCTCGTTTAGCCAGGTCTTGCATACG
GCCGCCAGAGCTTCCACTTGGTCAGGCAGTAGCTTGAAGTTTGCCTTTAGATCGTTATCCACGTGG
TACTTGTCCATCAACGCGCGCGCAGCCTCCATGCCCTTCTCCCACGCAGACACGATCGGCAGGCTC
AGCGGGTTTATCACCGTGCTTTCACTTTCCGCTTCACTGGACTCTTCCTTTTCCTCTTGCGTCCGC
ATACCCCGCGCCACTGGGTCGTCTTCATTCAGCCGCCGCACCGTGCGCTTACCTCCCTTGCCGTGC
TTGATTAGCACCGGTGGGTTGCTGAAACCCACCATTTGTAGCGCCACATCTTCTCTTTCTTCCTCG
CTGTCCACGATCACCTCTGGGGATGGCGGGCGCTCGGGCTTGGGAGAGGGGCGCTTCTTTTTCTTT
TTGGACGCAATGGCCAAATCCGCCGTCGAGGTCGATGGCCGCGGGCTGGGTGTGCGCGGCACCAGC
GCATCTTGTGACGAGTCTTCTTCGTCCTCGGACTCGAGACGCCGCCTCAGCCGCTTTTTTGGGGGC
GCGCGCTTGTCGTCATCGTCTTTGTAGTCGGGAGGCGGCGGCGACGGCGACGGGGACGACACGTCC
TCCATGGTTGGTGGACGTCGCGCCGCACCGCGTCCGCGCTCGGGGGTGGTTTCGCGCTGCTCCTCT
TCCCGACTGGCCATGGTGGCCGAGGATAACTTCGTATATGGTTTCTTATACGAAGTTATGATCCAG
ACATGATAAGATACATTGATGAGTTTGGACAAACCACAACTAGAATGCAGTGAAAAAAATGCTTTA
TTTGTGAAATTTGTGATGCTATTGCTTTATTTGTAACCATTATAAGCTGCAATAAACAAGTTAACA
ACAACAATTGCATTCATTTTATGTTTCAGGTTCAGGGGGAGGTGTGGGAGGTTTTTTAAAGCAAGT
AAAACCTCTACAAATGTGGTATGGCTGATTATGATCCTCTAGAGTCGCAGATCTGCTACGTATCAA
GCTGTGGCAGGGAAACCCTCTGCCTCCCCCGTGATGTAATACTTTTGCAAGGAATGCGATGAAGTA
GAGCCCGCAGTGGCCAAGTGGCTTTGGTCCGTCTCCTCCACGGATGCCCCTCCACGGCTAGTGGGC
GCATGTAGGCGGTGGGCGTCCGCCGCCTCCAGCAGCAGGTCATAGAGGGGCACCACGTTCTTGCAC
TTCATGCTGTACAGATGCTCCATGCCTTTGTTACTCATGTGTCGGATGTGGGAGAGGATGAGGAGG
AGCTGGGCCAGCCGCTGGTGCTGCTGCTGCAGGGTCAGGCCTGCCTTGGCCATCAGGTGGATCAAA
GTGTCTGTGATCTTGTCCAGGACTCGGTGGATATGGTCCTTCTCTTCCAGAGACTTCAGGGTGCTG
GACAGAAATGTGTACACTCCAGAATTAAGCAAAATAATAGATTTGAGGCACACAAACTCCTCTCCC
TGCAGATTCATCATGCGGAACCGAGATGATGTAGCCAGCAGCATGTCGAAGATCTCCACCATGCCC
TCTACACATTTTCCCTGGTTCCTGTCCAAGAGCAAGTTAGGAGCAAACAGTAGCTTCACTGGGTGC
TCCATGGAGCGCCAGACGAGACCAATCATCAGGATCTCTAGCCAGGCACATTCTAGAAGGTGGACC
TGATCATGGAGGGTCAAATCCACAAAGCCTGGCACCCTCTTCGCCCAGTTGATCATGTGAACCAGC
TCCCTGTCTGCCAGGTTGGTCAGTAAGCCCATCATCGAAGCTTCACTGAAGGGTCTGGTAGGATCA
TACTCGGAATAGAGTATGGGGGGCTCAGCATCCAACAAGGCACTGACCATCTGGTCGGCCGTCAGG
GACAAGGCCAGGCTGTTCTTCTTAGAGCGTTTGATCATGAGCGGGCTTGGCCAAAGGTTGGCAGCT
CTCATGTCTCCAGCAGATGGCTCGAGATCGCCATCTTCCAGCAGGCGCACCATTGCCCCTGTTTCA
CTATCCAGGTTACGGATATAGTTCATGACAATATTTACATTGGTCCAGCCACCAGCTTGCATGATC
TCCGGTATTGAAACTCCAGCGCGGGCCATATCTCGCGCGGCTCCGACACGGGCACTGTGTCCAGAC
CAGGCCAGGTATCTCTGACCAGAGTCATCCTAAAATACACAAACAATTAGAATCAGTAGTTTAACA
CATTATACACTTAAAAATTTTATATTTACCTTAGCGCCGTAAATCAATCGATGAGTTGCTTCAAAA
ATCCCTTCCAGGGCGCGAGTTGATAGCTGGCTGGTGGCAGATGGCGCGGCAACACCATTTTTTCTG
ACCCGGCAAAACAGGTAGTTATTCGGATCATCAGCTACACCAGAGACGGAAATCCATCGCTCGACC
AGTTTAGTGACTCCCAGGCTAAGTGCCTTCTCTACACCTGCGGTGCTAACCAGCGTTTTCGTTCTG
CCAATATGGATTAACATTCTCCCACCGTCAGTACGTGAGATATCTTTAACCCTGATCCTGGCAATT
TCGGCTATACGTAACAGGGTGTTATAAGCAATCCCCAGAAATGCCAGATTACGTATATCCTGGCAG
CGATCGCTATTTTCCATGAGTGAACGGACTTGGTCGAAATCAGTGCGTTCGAACGCTAGAGCCTGT
TTTGCACGTTCACCGGCATCAACGTTTTCTTTTCGGATCCGCCGCATAACCAGTGAAACAGCATTG
CTGTCACTTGGTCGTGGCAGCCCGGACCGACGATGAAGCATGTTTAGCTGGCCCAAATGTTGCTGG
ATAGTTTTTACTGCCAGACCGCGCGCTTGAAGATATAGAAGATAATCGCGAACATCTTCAGGTTCT
GCGGGAAACCATTTCCGGTTATTCAACTTGCACCATGCCGCCCACGACCGGCAAACGGACAGAAGC
ATTTTCCAGGTATGCTCAGAAAACGCCTGGCGATCCCTGAACATGTCCATCAGGTTCTTGCGAACC
TCATCACTCGTTGCATCGACCGGTAATGCAGGCAAATTTTGGTGTACGGTCAGTAAATTGGACATG
GTGGCTACGTAATAACTTCGTATATGGTTTCTTATACGAAGTTATGCGGCCGCTTTACGAGGGTAG
GAAGTGGTACGGAAAGTTGGTATAAGACAAAAGTGTTGTGGAATTGCTCCAGGCGATCTGACGGTT
CACTAAACGAGCTCTGCTTTTATAGGCGCCCACCGTACACGCCTAAAGCTTATACGTTCTCTATCA
CTGATAGGGAGTAAACTGGATATACGTTCTCTATCACTGATAGGGAGTAAACTGTAGATACGTTCT
CTATCACTGATAGGGAGTAAACTGGTCATACGTTCTCTATCACTGATAGGGAGTAAACTCCTTATA
CGTTCTCTATCACTGATAGGGAGTAAAGTCTGCATACGTTCTCTATCACTGATAGGGAGTAAACTC
TTCATACGTTCTCTATCACTGATAGGGAGTAAACTCGCGGCCGCAGAGAAATGTTCTGGCACCTGC
ACTTGCACTGGGGACAGCCTATTTTGCTAGTTTGTTTTGTTTCGTTTTGTTTTGATGGAGAGCGTA
TGTTAGTACTATCGATTCACACAAAAAACCAACACACAGATGTAATGAAAATAAAGATATTTTATT
GGATCTGCGATCGCTCCGGTGCCCGTCAGTGGGCAGAGCGCACATCGCCCACAGTCCCCGAGAAGT
TGGGGGGAGGGGTCGGCAATTGAACGGGTGCCTAGAGAAGGTGGCGCGGGGTAAACTGGGAAAGTG
ATGTCGTGTACTGGCTCCGCCTTTTTCCCGAGGGTGGGGGAGAACCGTATGTAAGTGCAGTAGTCG
CCGTGAACGTTCTTTTTCGCAACGGGTTTGCCGCCAGAACACAGCTGAAGCTTCGAGGGGCTCGCA
TCTCTCCTTCACGCGCCCGCCGCCCTACCTGAGGCCGCCATCCACGCCGGTTGAGTCGCGTTCTGC
CGCCTCCCGCCTGTGGTGCCTCCTGAACTGCGTCCGCCGTCTAGGTAAGTTTAAAGCTCAGGTCGA
GACCGGGCCTTTGTCCGGCGCTCCCTTGGAGCCTACCTAGACTCAGCCGGCTCTCCACGCTTTGCC
TGACCCTGCTTGCTCAACTCTACGTCTTTGTTTCGTTTTCTGTTCTGCGCCGTTACAGATCCAAGC
TGTGACCGGCGCCTACGCTAGCGGATCCGCCGCCACCATGTCTAGACTGGACAAGAGCAAAGTCAT
AAACTCTGCTCTGGAATTACTCAATGGAGTCGGTATCGAAGGCCTGACGACAAGGAAACTCGCTCA
AAAGCTGGGAGTTGAGCAGCCTACCCTGTACTGGCACGTGAAGAACAAGCGGGCCCTGCTCGATGC
CCTGCCAATCGAGATGCTGGACAGGCATCATACCCACTCCTGCCCCCTGGAAGGCGAGTCATGGCA
AGACTTTCTGCGGAACAACGCCAAGTCATACCGCTGTGCTCTTCTCTCACATCGCGACGGGGCTAA
AGTGCATCTCGGCACCCGCCCAACAGAGAAACAGTACGAAACCCTGGAAAATCAGCTCGCGTTCCT
GTGTCAGCAAGGCTTCTCCCTGGAGAACGCACTGTACGCTCTGTCCGCCGTGGGCCACTTTACACT
GGGCTGCGTATTGGAGGAACAGGAGCATCAAGTAGCAAAAGAGGAAAGAGAGACACCTACCACCGA
TTCTATGCCCCCACTTCTGAAACAAGCAATTGAGCTGTTCGACCGGCAGGGAGCCGAACCTGCCTT
CCTTTTCGGCCTGGAACTAATCATATGTGGCCTGGAGAAACAGCTAAAGTGCGAAAGCGGCGGGCC
GACCGACGCCCTTGACGATTTTGACTTAGACATGCTCCCAGCCGATGCCCTTGACGACTTTGACCT
TGATATGCTGCCTGCTGACGCTCTTGACGATTTTGACCTTGACATGCTCCCCGGGTGAACCGGTCG
CTGATCAGCCTCGACTGTGCCTTCTAGTTGCCAGCCATCTGTTGTTTGCCCCTCCCCCGTGCCTTC
CTTGACCCTGGAAGGTGCCACTCCCACTGTCCTTTCCTAATAAAATGAGGAAATTGCATCGCATTG
TCTGAGTAGGTGTCATTCTATTCTGGGGGGTGGGGTGGGGCAGGACAGCAAGGGGGAGGATTGGGA
AGACAATAGCAGGCATGCTGGGGATGCGGTGGGCTCTATGGCTTCTGAGGCGGAAAGAACCAGCTG
GGGCTCGACTAGAGCTTGCGGAACCCTTAGAGGGCCTATTTCCCATGATTCCTTCATATTTGCATA
TACGATACAAGGCTGTTAGAGAGATAATTAGAATTAATTTGACTGTAAACACAAAGATATTAGTAC
AAAATAATAACTTCGTATAATGTATGCTATACGAAGTTATCAGACATGATAAGATACATTGATGAG
TTTGGACAAACCACAACTAGAATGCAGTGAAAAAAATGCTTTATTTGTGAAATTTGTGATGCTATT
GCTTTATTTGTAACCATTATAAGCTGCAATAAACAAGGTACCTCAAGCGCCGGGTTTTCGCGTCAT
GCACCACGTCCGTGGTAGAACTAGTATTATGCCCAGTACATGACCTTATGGGACTTTCCTACTTGG
CAGTACATCTACGTATTAGTCATCGCTATTACCATGGTGATGCGGTTTTGGCAGTACATCAATGGG
CGTGGATAGCGGTTTGACTCACGGGGATTTCCAAGTCTCCACCCCATTGACGTCAATGGGAGTTTG
TTTTGGCACCAAAATCAACGGGACTTTCCAAAATGTCGTAACAACTCCGCCCCATTGACGCAAATG
GGCGGTAGGCGTGTACGGTGGGAGGTTTATATAAGCAGAGCTCGTTTAGTGAACCGTCAGATCGCC
TGGAGACGCCATCCACGCTGTTTTGACCTCCATAGAAGAGAATTCGCCGCCACCATGACAGAATAT
AAACCTACTGTCAGACTGGCAACTCGAGACGACGTCCCTAGGGCCGTGAGAACATTGGCTGCCGCT
TTCGCGGATTATCCCGCTACACGCCACACAGTTGATCCTGATAGACATATTGAACGGGTTACAGAA
TTGCAAGAACTTTTTTTGACCAGGGTAGGATTGGACATCGGTAAAGTTTGGGTCGCCGACGACGGG
GCTGCAGTGGCAGTGTGGACGACTCCGGAGAGCGTTGAGGCCGGGGCTGTATTTGCAGAAATTGGT
CCCCGAATGGCTGAGCTTAGTGGCTCTCGTCTCGCGGCTCAGCAACAAATGGAAGGACTCCTCGCC
CCTCACCGCCCTAAAGAACCAGCTTGGTTCCTCGCTACTGTGGGCGTTAGCCCCGATCATCAGGGA
AAGGGCCTTGGTTCCGCGGTGGTATTGCCCGGAGTAGAAGCCGCAGAACGAGCCGGAGTGCCAGCC
TTTCTTGAAACGTCAGCGCCAAGGAATTTGCCCTTCTATGAACGGCTCGGATTTACAGTTACTGCT
GACGTTGAAGTACCCGAGGGCCCACGGACGTGGTGCATGACGCGAAAACCCGGCGCTTGAACCGGT
CGCTGATCAGCCTCGACTGTGCCTTCTAGTTGCCAGCCATCTGTTGTTTGCCCCTCCCCCGTGCCT
TCCTTGACCCTGGAAGGTGCCACTCCCACTGTCCTTTCCTAATAAAATGAGGAAATTGCATCGCAT
TGTCTGAGTAGGTGTCATTCTATTCTGGGGGGTGGGGTGGGGCAGGACAGCAAGGGGGAGGATTGG
GAAGACAATAGCAGGCATGCTGGGGATGCGGTGGGCTCTATGGCTTCTGAGGCGGAAAGAACCAGC
TGGGGCTCGACTAGAGCTTGCGGAACCCTTAGGTTGGGAAAAGCGCTCCCCTACCCATAACTTCGT
ATAATGTATGCTATACGAAGTTATTTTGCAGTTTTAAAATTATGTTTTAAAATGGACTATCATATG
CTTACCGTAACTTGAAAGTATTTCGATTTCTTGGCTTTATATATCTTGTGGAAAGGACGAAACACC
GGGCACTCTTCCGTGATCTGGTGGATAAATTCGCAAGGGTATCATGGCGGACGACCGGGATTCGAA
CCCCGGATCCGGCCGTCCGCCGTGATCCATGCGGTTACCGCCCGCGTGTCGAACCCAGGTGTGCGA
CGTCAGACAACGGGGGAGCGCTCCTTTTTGGGCCCAT
SEQ ID NO: 28 (STXC0125)
TGGTATGGCTTTTTCCCCGTATCCCCCCAGGTGTCTGCAGGCTCAAAGAGCAGCGAGAAGCGTTCA
GAGGAAAGCGATCCCGTGCCACCTTCCCCGTGCCCGGGCTGTCCCCGCACGCTGCCGGCTCGGGGA
TGCGGGGGGAGCGCCGGACCGGAGCGGAGCCCCGGGCGGCTCGCTGCTGCCCCCTAGCGGGGGAGG
GACGTAATTACATCCCTGGGGGCTTTGGGGGGGGGCTGTCCCTGATATCTATAACAAGAAAATATA
TATATAATAAGTTATCACGTAAGTAGAACATGAAATAACAATATAATTATCGTATGAGTTAAATCT
TAAAAGTCACGTAAAAGATAATCATGCGTCATTTTGACTCACGCGGTCGTTATAGTTCAAAATCAG
TGACACTTACCGCATTGACAAGCACGCCTCACGGGAGCTCCAAGCGGCGACTGAGATGTCCTAAAT
GCACAGCGACGGATTCGCGCTATTTAGAAAGAGAGAGCAATATTTCAAGAATGCATGCGTCAATTT
TACGCAGACTATCTTTCTAGGGTTAATCTAGCTGCATCAGGATCATATCGTCGGGTCTTTTTTCCG
GCTCAGTCATCGCCCAAGCTGGCGCTATCTGGGCATCGGGGAGGAAGAAGCCCGTGCCTTTTCCCG
CGAGGTTGAAGCGGCATGGAAAGAGTTTGCCGAGGATGACTGCTGCTGCATTGACGTTGAGCGAAA
ACGCACGTTTACCATGATGATTCGGGAAGGTGTGGCCATGCACGCCTTTAACGGTGAACTGTTCGT
TCAGGCCACCTGGGATACCAGTTCGTCGCGGCTTTTCCGGACACAGTTCCGGATGGTCAGCCCGAA
GCGCATCAGCAACCCGAACAATACCGGCGACAGCCGGAACTGCCGTGCCGGTGTGCAGATTAATGA
CAGCGGTGCGGCGCTGGGATATTACGTCAGCGAGGACGGGTATCCTGGCTGGATGCCGCAGAAATG
GACATGGATACCCCGTGAGTTACCCGGCGGGCGCGCTTGGCGTAATCATGGTCATAGCTGTTTCCT
GTGTGAAATTGTTATCCGCTCACAATTCCACACAACATACGAGCCGGAAGCATAAAGTGTAAAGCC
TGGGGTGCCTAATGAGTGAGCTAACTCACATTAATTGCGTTGCGCTCACTGCCCGCTTTCCAGTCG
GGAAACCTGTCGTGCCAGCTGCATTAATGAATCGGCCAACGCGCGGGGAGAGGCGGTTTGCGTATT
GGGCGCTCTTCCGCTTCCTCGCTCACTGACTCGCTGCGCTCGGTCGTTCGGCTGCGGCGAGCGGTA
TCAGCTCACTCAAAGGCGGTAATACGGTTATCCACAGAATCAGGGGATAACGCAGGAAAGAACATG
TGAGCAAAAGGCCAGCAAAAGGCCAGGAACCGTAAAAAGGCCGCGTTGCTGGCGTTTTTCCATAGG
CTCCGCCCCCCTGACGAGCATCACAAAAATCGACGCTCAAGTCAGAGGTGGCGAAACCCGACAGGA
CTATAAAGATACCAGGCGTTTCCCCCTGGAAGCTCCCTCGTGCGCTCTCCTGTTCCGACCCTGCCG
CTTACCGGATACCTGTCCGCCTTTCTCCCTTCGGGAAGCGTGGCGCTTTCTCATAGCTCACGCTGT
AGGTATCTCAGTTCGGTGTAGGTCGTTCGCTCCAAGCTGGGCTGTGTGCACGAACCCCCCGTTCAG
CCCGACCGCTGCGCCTTATCCGGTAACTATCGTCTTGAGTCCAACCCGGTAAGACACGACTTATCG
CCACTGGCAGCAGCCACTGGTAACAGGATTAGCAGAGCGAGGTATGTAGGCGGTGCTACAGAGTTC
TTGAAGTGGTGGCCTAACTACGGCTACACTAGAAGGACAGTATTTGGTATCTGCGCTCTGCTGAAG
CCAGTTACCTTCGGAAAAAGAGTTGGTAGCTCTTGATCCGGCAAACAAACCACCGCTGGTAGCGGT
GGTTTTTTTGTTTGCAAGCAGCAGATTACGCGCAGAAAAAAAGGATCTCAAGAAGATCCTTTGATC
TTTTCTACGGGGTCTGACGCTCAGTGGAACGAAAACTCACGTTAAGGGATTTTGGTCATGAGATTA
TCAAAAAGGATCTTCACCTAGATCCTTTTAAATTAAAAATGAAGTTTTAAATCAATCTAAAGTATA
TATGAGTAAACTTGGTCTGACAGTTACCAATGCTTAATCAGTGAGGCACCTATCTCAGCGATCTGT
CTATTTCGTTCATCCATAGTTGCCTGACTCCCCGTCGTGTAGATAACTACGATACGGGAGGGCTTA
CCATCTGGCCCCAGTGCTGCAATGATACCGCGAGACCCACGCTCACCGGCTCCAGATTTATCAGCA
ATAAACCAGCCAGCCGGAAGGGCCGAGCGCAGAAGTGGTCCTGCAACTTTATCCGCCTCCATCCAG
TCTATTAATTGTTGCCGGGAAGCTAGAGTAAGTAGTTCGCCAGTTAATAGTTTGCGCAACGTTGTT
GCCATTGCTACAGGCATCGTGGTGTCACGCTCGTCGTTTGGTATGGCTTCATTCAGCTCCGGTTCC
CAACGATCAAGGCGAGTTACATGATCCCCCATGTTGTGCAAAAAAGCGGTTAGCTCCTTCGGTCCT
CCGATCGTTGTCAGAAGTAAGTTGGCCGCAGTGTTATCACTCATGGTTATGGCAGCACTGCATAAT
TCTCTTACTGTCATGCCATCCGTAAGATGCTTTTCTGTGACTGGTGAGTACTCAACCAAGTCATTC
TGAGAATAGTGTATGCGGCGACCGAGTTGCTCTTGCCCGGCGTCAATACGGGATAATACCGCGCCA
CATAGCAGAACTTTAAAAGTGCTCATCATTGGAAAACGTTCTTCGGGGCGAAAACTCTCAAGGATC
TTACCGCTGTTGAGATCCAGTTCGATGTAACCCACTCGTGCACCCAACTGATCTTCAGCATCTTTT
ACTTTCACCAGCGTTTCTGGGTGAGCAAAAACAGGAAGGCAAAATGCCGCAAAAAAGGGAATAAGG
GCGACACGGAAATGTTGAATACTCATACTCTTCCTTTTTCAATATTATTGAAGCATTTATCAGGGT
TATTGTCTCATGAGCGGATACATATTTGAATGTATTTAGAAAAATAAACAAATAGGGGTTCCGCGC
ACATTTCCCCGAAAAGTGCCACCTAAATTGTAAGCGTTAATATTTTGTTAAAATTCGCGTTAAATT
TTTGTTAAATCAGCTCATTTTTTAACCAATAGGCCGAAATCGGCAAAATCCCTTATAAATCAAAAG
AATAGACCGAGATAGGGTTGAGTGTTGTTCCAGTTTGGAACAAGAGTCCACTATTAAAGAACGTGG
ACTCCAACGTCAAAGGGCGAAAAACCGTCTATCAGGGCGATGGCCCACTACGTGAACCATCACCCT
AATCAAGTTTTTTGGGGTCGAGGTGCCGTAAAGCACTAAATCGGAACCCTAAAGGGAGCCCCCGAT
TTAGAGCTTGACGGGGAAAGCCGGCGAACGTGGCGAGAAAGGAAGGGAAGAAAGCGAAAGGAGCGG
GCGCTAGGGCGCTGGCAAGTGTAGCGGTCACGCTGCGCGTAACCACCACACCCGCCGCGCTTAATG
CGCCGCTACAGGGCGCGTCCCATTCGCCATTCAGGCTGCGCAACTGTTGGGAAGGGCGATCGGTGC
GGGCCTCTTCGCTATTACGCCAGCTGGCGAAAGGGGGATGTGCTGCAAGGCGATTAAGTTGGGTAA
CGCCAGGGTTTTCCCAGTCACGACGTTGTAAAACGACGGCCAGTGAGCGCGCCTCGTTCATTCACG
TTTTTGAACCCGTGGAGGACGGGCAGACTCGCGGTGCAAATGTGTTTTACAGCGTGATGGAGCAGA
TGAAGATGCTCGACACGCTGCAGAACACGCAGCTAGATTAACCCTAGAAAGATAATCATATTGTGA
CGTACGTTAAAGATAATCATGTGTAAAATTGACGCATGTGTTTTATCGGTCTGTATATCGAGGTTT
ATTTATTAATTTGAATAGATATTAAGTTTTATTATATTTACACTTACATACTAATAATAAATTCAA
CAAACAATTTATTTATGTTTATTTATTTATTAAAAAAAACAAAAACTCAAAATTTCTTCTATAAAG
TAACAAAACTTTTATGAGGGACAGCCCCCCCCCAAAGCCCCCAGGGATGTAATTACGTCCCTCCCC
CGCTAGGGGGCAGCAGCGAGCCGCCCGGGGCTCCGCTCCGGTCCGGCGCTCCCCCCGCATCCCCGA
GCCGGCAGCGTGCGGGGACAGCCCGGGCACGGGGAAGGTGGCACGGGATCGCTTTCCTCTGAACGC
TTCTCGCTGCTCTTTGAGCCTGCAGACACCTGGGGGGATACGGGGAAAAGGCCTCCACGGCCACTA
GTCCATAGAGCCCACCGCATCCCCAGCATGCCTGCTATTGTCTTCCCAATCCTCCCCCTTGCTGTC
CTGCCCCACCCCACCCCCTAGAATAGAATGACACCTACTCAGACAATGCGATGCAATTTCCTCATT
TTATTAGGAAAGGACAGTGGGAGTGGCACCTTCCAGGGTCAAGGAAGGCACGGGGGAGGGGCAAAC
AACAGATGGCTGGCAACTAGAAGGCACAGCTACATGGGGGTAGAGTCATAATCGTGCATCAGGATA
GGGCGGTGGTGCTGCAGCAGCGCGCGAATAAACTGCTGCCGCCGCCGCTCCGTCCTGCAGGAATAC
AACATGGCAGTGGTCTCCTCAGCGATGATTCGCACCGCCCGCAGCATGAGACGCCTTGTCCTCCGG
GCACAGCAGCGCACCCTGATCTCACTTAAATCAGCACAGTAACTGCAGCACAGCACCACAATATTG
TTCAAAATCCCACAGTGCAAGGCGCTGTATCCAAAGCTCATGGCGGGGACCACAGAACCCACGTGG
CCATCATACCACAAGCGCAGGTAGATTAAGTGGCGACCCCTCATAAACACGCTGGACATAAACATT
ACCTCTTTTGGCATGTTGTAATTCACCACCTCCCGGTACCATATAAACCTCTGATTAAACATGGCG
CCATCCACCACCATCCTAAACCAGCTGGCCAAAACCTGCCCGCCGGCTATGCACTGCAGGGAACCG
GGACTGGAACAATGACAGTGGAGAGCCCAGGACTCGTAACCATGGATCATCATGCTCGTCATGATA
TCAATGTTGGCACAACACAGGCACACGTGCATACACTTCCTCAGGATTACAAGCTCCTCCCGCGTC
AGAACCATATCCCAGGGAACAACCCATTCCTGAATCAGCGTAAATCCCACACTGCAGGGAAGACCT
CGCACGTAACTCACGTTGTGCATTGTCAAAGTGTTACATTCGGGCAGCAGCGGATGATCCTCCAGT
ATGGTAGCGCGGGTCTCTGTCTCAAAAGGAGGTAGGCGATCCCTACTGTACGGAGTGCGCCGAGAC
AACCGAGATCGTGTTGGTCGTAGTGTCATGCCAAATGGAACGCCGGACGTAGTCATGGTTGTGGCC
ATATTATCATCGTGTTTTTCAAAGGAAAACCACGTCCCCGTGGTTCGGGGGGCCTAGACGTTTTTT
TAACCTCGACTAAACACATGTAAAGCATGTGCACCGAGGCCCCAGATCAGATCCCATACAATGGGG
TACCTTCTGGGCATCCTTCAGCCCCTTGTTGAATACGCTTGAGGAGAGCCATTTGACTCTTTCCAC
AACTATCCAACTCACAACGTGGCACTGGGGTTGTGCCGCCTTTGCAGGTGTATCTTATACACGTGG
CTTTTGGCCGCAGAGGCACCTGTCGCCAGGTGGGGGGTTCCGCTGCCTGCAAAGGGTCGCTACAGA
CGTTGTTTGTCTTCAAGAAGCTTCCAGAGGAACTGCTTCCTTCACGACATTCAACAGACCTTGCAT
TCCTTTGGCGAGAGGGGAAAGACCCCTAGGAATGCTCGTCAAGAAGACAGGGCCAGGTTTCCGGGC
CCTCACATTGCCAAAAGACGGCAATATGGTGGAAAATAACATATAGACAAACGCACACCGGCCTTA
TTCCAAGCGGCTTCGGCCAGTAACGTTAGGGGGGGGGGAGGGAGAGGGGCTTAAAAATCAAAGGGG
TTCTGCCGCGCATCACTATGCGCCACTGGCAGGGACACGTTGCGATACTGGTGTTTAGTGCTCCAC
TTAAACTCAGGCACAACCATCCGCGGCAGCTCGGTGAAGTTTTCACTCCACAGGCTGCGCACCATC
ACCAACGCGTTTAGCAGGTCGGGCGCCGATATCTTGAAGTCGCAGTTGGGGCCTCCGCCCTGCGCG
CGCGAGTTGCGATACACAGGGTTGCAGCACTGGAACACTATCAGCGCCGGGTGGTGCACGCTGGCC
AGCACGCTCTTGTCGGAGATCAGATCCGCGTCCAGGTCCTCCGCGTTGCTCAGGGCGAACGGAGTC
AACTTTGGTAGCTGCCTTCCCAAAAAGGGTGCATGCCCAGGCTTTGAGTTGCACTCGCACCGTAGT
GGCATCAGAAGGTGACCGTGCCCGGTCTGGGCGTTAGGATACAGCGCCTGCATGAAAGCCTTGATC
TGCTTAAAAGCCACCTGAGCCTTTGCGCCTTCAGAGAAGAACATGCCGCAAGACTTGCCGGAAAAC
TGATTGGCCGGACAGGCCGCGTCATGCACGCAGCACCTTGCGTCGGTGTTGGAGATCTGCACCACA
TTTCGGCCCCACCGGTTCTTCACGATCTTGGCCTTGCTAGACTGCTCCTTCAGCGCGCGCTGCCCG
TTTTCGCTCGTCACATCCATTTCAATCACGTGCTCCTTATTTATCATAATGCTCCCGTGTAGACAC
TTAAGCTCGCCTTCGATCTCAGCGCAGCGGTGCAGCCACAACGCGCAGCCCGTGGGCTCGTGGTGC
TTGTAGGTTACCTCTGCAAACGACTGCAGGTACGCCTGCAGGAATCGCCCCATCATCGTCACAAAG
GTCTTGTTGCTGGTGAAGGTCAGCTGCAACCCGCGGTGCTCCTCGTTTAGCCAGGTCTTGCATACG
GCCGCCAGAGCTTCCACTTGGTCAGGCAGTAGCTTGAAGTTTGCCTTTAGATCGTTATCCACGTGG
TACTTGTCCATCAACGCGCGCGCAGCCTCCATGCCCTTCTCCCACGCAGACACGATCGGCAGGCTC
AGCGGGTTTATCACCGTGCTTTCACTTTCCGCTTCACTGGACTCTTCCTTTTCCTCTTGCGTCCGC
ATACCCCGCGCCACTGGGTCGTCTTCATTCAGCCGCCGCACCGTGCGCTTACCTCCCTTGCCGTGC
TTGATTAGCACCGGTGGGTTGCTGAAACCCACCATTTGTAGCGCCACATCTTCTCTTTCTTCCTCG
CTGTCCACGATCACCTCTGGGGATGGCGGGCGCTCGGGCTTGGGAGAGGGGCGCTTCTTTTTCTTT
TTGGACGCAATGGCCAAATCCGCCGTCGAGGTCGATGGCCGCGGGCTGGGTGTGCGCGGCACCAGC
GCATCTTGTGACGAGTCTTCTTCGTCCTCGGACTCGAGACGCCGCCTCAGCCGCTTTTTTGGGGGC
GCGCGCTTGTCGTCATCGTCTTTGTAGTCGGGAGGCGGCGGCGACGGCGACGGGGACGACACGTCC
TCCATGGTTGGTGGACGTCGCGCCGCACCGCGTCCGCGCTCGGGGGTGGTTTCGCGCTGCTCCTCT
TCCCGACTGGCCATGGTGGCCGAGGATAACTTCGTATATGGTTTCTTATACGAAGTTATGATCCAG
ACATGATAAGATACATTGATGAGTTTGGACAAACCACAACTAGAATGCAGTGAAAAAAATGCTTTA
TTTGTGAAATTTGTGATGCTATTGCTTTATTTGTAACCATTATAAGCTGCAATAAACAAGTTAACA
ACAACAATTGCATTCATTTTATGTTTCAGGTTCAGGGGGAGGTGTGGGAGGTTTTTTAAAGCAAGT
AAAACCTCTACAAATGTGGTATGGCTGATTATGATCCTCTAGAGTCGCAGATCTGCTACGTATCAA
GCTGTGGCAGGGAAACCCTCTGCCTCCCCCGTGATGTAATACTTTTGCAAGGAATGCGATGAAGTA
GAGCCCGCAGTGGCCAAGTGGCTTTGGTCCGTCTCCTCCACGGATGCCCCTCCACGGCTAGTGGGC
GCATGTAGGCGGTGGGCGTCCGCCGCCTCCAGCAGCAGGTCATAGAGGGGCACCACGTTCTTGCAC
TTCATGCTGTACAGATGCTCCATGCCTTTGTTACTCATGTGTCGGATGTGGGAGAGGATGAGGAGG
AGCTGGGCCAGCCGCTGGTGCTGCTGCTGCAGGGTCAGGCCTGCCTTGGCCATCAGGTGGATCAAA
GTGTCTGTGATCTTGTCCAGGACTCGGTGGATATGGTCCTTCTCTTCCAGAGACTTCAGGGTGCTG
GACAGAAATGTGTACACTCCAGAATTAAGCAAAATAATAGATTTGAGGCACACAAACTCCTCTCCC
TGCAGATTCATCATGCGGAACCGAGATGATGTAGCCAGCAGCATGTCGAAGATCTCCACCATGCCC
TCTACACATTTTCCCTGGTTCCTGTCCAAGAGCAAGTTAGGAGCAAACAGTAGCTTCACTGGGTGC
TCCATGGAGCGCCAGACGAGACCAATCATCAGGATCTCTAGCCAGGCACATTCTAGAAGGTGGACC
TGATCATGGAGGGTCAAATCCACAAAGCCTGGCACCCTCTTCGCCCAGTTGATCATGTGAACCAGC
TCCCTGTCTGCCAGGTTGGTCAGTAAGCCCATCATCGAAGCTTCACTGAAGGGTCTGGTAGGATCA
TACTCGGAATAGAGTATGGGGGGCTCAGCATCCAACAAGGCACTGACCATCTGGTCGGCCGTCAGG
GACAAGGCCAGGCTGTTCTTCTTAGAGCGTTTGATCATGAGCGGGCTTGGCCAAAGGTTGGCAGCT
CTCATGTCTCCAGCAGATGGCTCGAGATCGCCATCTTCCAGCAGGCGCACCATTGCCCCTGTTTCA
CTATCCAGGTTACGGATATAGTTCATGACAATATTTACATTGGTCCAGCCACCAGCTTGCATGATC
TCCGGTATTGAAACTCCAGCGCGGGCCATATCTCGCGCGGCTCCGACACGGGCACTGTGTCCAGAC
CAGGCCAGGTATCTCTGACCAGAGTCATCCTAAAATACACAAACAATTAGAATCAGTAGTTTAACA
CATTATACACTTAAAAATTTTATATTTACCTTAGCGCCGTAAATCAATCGATGAGTTGCTTCAAAA
ATCCCTTCCAGGGCGCGAGTTGATAGCTGGCTGGTGGCAGATGGCGCGGCAACACCATTTTTTCTG
ACCCGGCAAAACAGGTAGTTATTCGGATCATCAGCTACACCAGAGACGGAAATCCATCGCTCGACC
AGTTTAGTGACTCCCAGGCTAAGTGCCTTCTCTACACCTGCGGTGCTAACCAGCGTTTTCGTTCTG
CCAATATGGATTAACATTCTCCCACCGTCAGTACGTGAGATATCTTTAACCCTGATCCTGGCAATT
TCGGCTATACGTAACAGGGTGTTATAAGCAATCCCCAGAAATGCCAGATTACGTATATCCTGGCAG
CGATCGCTATTTTCCATGAGTGAACGGACTTGGTCGAAATCAGTGCGTTCGAACGCTAGAGCCTGT
TTTGCACGTTCACCGGCATCAACGTTTTCTTTTCGGATCCGCCGCATAACCAGTGAAACAGCATTG
CTGTCACTTGGTCGTGGCAGCCCGGACCGACGATGAAGCATGTTTAGCTGGCCCAAATGTTGCTGG
ATAGTTTTTACTGCCAGACCGCGCGCTTGAAGATATAGAAGATAATCGCGAACATCTTCAGGTTCT
GCGGGAAACCATTTCCGGTTATTCAACTTGCACCATGCCGCCCACGACCGGCAAACGGACAGAAGC
ATTTTCCAGGTATGCTCAGAAAACGCCTGGCGATCCCTGAACATGTCCATCAGGTTCTTGCGAACC
TCATCACTCGTTGCATCGACCGGTAATGCAGGCAAATTTTGGTGTACGGTCAGTAAATTGGACATG
GTGGCTACGTAATAACTTCGTATATGGTTTCTTATACGAAGTTATGCGGCCGCTTTACGAGGGTAG
GAAGTGGTACGGAAAGTTGGTATAAGACAAAAGTGTTGTGGAATTGCTCCAGGCGATCTGACGGTT
CACTAAACGAGCTCTGCTTTTATAGGCGCCCACCGTACACGCCTAAAGCTTATACGTTCTCTATCA
CTGATAGGGAGTAAACTGGATATACGTTCTCTATCACTGATAGGGAGTAAACTGTAGATACGTTCT
CTATCACTGATAGGGAGTAAACTGGTCATACGTTCTCTATCACTGATAGGGAGTAAACTCCTTATA
CGTTCTCTATCACTGATAGGGAGTAAAGTCTGCATACGTTCTCTATCACTGATAGGGAGTAAACTC
TTCATACGTTCTCTATCACTGATAGGGAGTAAACTCGCGGCCGCAGAGAAATGTTCTGGCACCTGC
ACTTGCACTGGGGACAGCCTATTTTGCTAGTTTGTTTTGTTTCGTTTTGTTTTGATGGAGAGCGTA
TGTTAGTACTATCGATTCACACAAAAAACCAACACACAGATGTAATGAAAATAAAGATATTTTATT
GGATCTGCGATCGCTCCGGTGCCCGTCAGTGGGCAGAGCGCACATCGCCCACAGTCCCCGAGAAGT
TGGGGGGAGGGGTCGGCAATTGAACGGGTGCCTAGAGAAGGTGGCGCGGGGTAAACTGGGAAAGTG
ATGTCGTGTACTGGCTCCGCCTTTTTCCCGAGGGTGGGGGAGAACCGTATGTAAGTGCAGTAGTCG
CCGTGAACGTTCTTTTTCGCAACGGGTTTGCCGCCAGAACACAGCTGAAGCTTCGAGGGGCTCGCA
TCTCTCCTTCACGCGCCCGCCGCCCTACCTGAGGCCGCCATCCACGCCGGTTGAGTCGCGTTCTGC
CGCCTCCCGCCTGTGGTGCCTCCTGAACTGCGTCCGCCGTCTAGGTAAGTTTAAAGCTCAGGTCGA
GACCGGGCCTTTGTCCGGCGCTCCCTTGGAGCCTACCTAGACTCAGCCGGCTCTCCACGCTTTGCC
TGACCCTGCTTGCTCAACTCTACGTCTTTGTTTCGTTTTCTGTTCTGCGCCGTTACAGATCCAAGC
TGTGACCGGCGCCTACGCTAGCGGATCCGCCGCCACCATGTCTAGACTGGACAAGAGCAAAGTCAT
AAACTCTGCTCTGGAATTACTCAATGGAGTCGGTATCGAAGGCCTGACGACAAGGAAACTCGCTCA
AAAGCTGGGAGTTGAGCAGCCTACCCTGTACTGGCACGTGAAGAACAAGCGGGCCCTGCTCGATGC
CCTGCCAATCGAGATGCTGGACAGGCATCATACCCACTCCTGCCCCCTGGAAGGCGAGTCATGGCA
AGACTTTCTGCGGAACAACGCCAAGTCATACCGCTGTGCTCTTCTCTCACATCGCGACGGGGCTAA
AGTGCATCTCGGCACCCGCCCAACAGAGAAACAGTACGAAACCCTGGAAAATCAGCTCGCGTTCCT
GTGTCAGCAAGGCTTCTCCCTGGAGAACGCACTGTACGCTCTGTCCGCCGTGGGCCACTTTACACT
GGGCTGCGTATTGGAGGAACAGGAGCATCAAGTAGCAAAAGAGGAAAGAGAGACACCTACCACCGA
TTCTATGCCCCCACTTCTGAAACAAGCAATTGAGCTGTTCGACCGGCAGGGAGCCGAACCTGCCTT
CCTTTTCGGCCTGGAACTAATCATATGTGGCCTGGAGAAACAGCTAAAGTGCGAAAGCGGCGGGCC
GACCGACGCCCTTGACGATTTTGACTTAGACATGCTCCCAGCCGATGCCCTTGACGACTTTGACCT
TGATATGCTGCCTGCTGACGCTCTTGACGATTTTGACCTTGACATGCTCCCCGGGTGAACCGGTCG
CTGATCAGCCTCGACTGTGCCTTCTAGTTGCCAGCCATCTGTTGTTTGCCCCTCCCCCGTGCCTTC
CTTGACCCTGGAAGGTGCCACTCCCACTGTCCTTTCCTAATAAAATGAGGAAATTGCATCGCATTG
TCTGAGTAGGTGTCATTCTATTCTGGGGGGTGGGGTGGGGCAGGACAGCAAGGGGGAGGATTGGGA
AGACAATAGCAGGCATGCTGGGGATGCGGTGGGCTCTATGGCTTCTGAGGCGGAAAGAACCAGCTG
GGGCTCGACTAGAGCTTGCGGAACCCTTAGAGGGCCTATTTCCCATGATTCCTTCATATTTGCATA
TACGATACAAGGCTGTTAGAGAGATAATTAGAATTAATTTGACTGTAAACACAAAGATATTAGTAC
AAAATAATAACTTCGTATAATGTATGCTATACGAAGTTATCAGACATGATAAGATACATTGATGAG
TTTGGACAAACCACAACTAGAATGCAGTGAAAAAAATGCTTTATTTGTGAAATTTGTGATGCTATT
GCTTTATTTGTAACCATTATAAGCTGCAATAAACAAGGTACCTCAAGCGCCGGGTTTTCGCGTCAT
GCACCACGTCCGTGGTCAACCCTCCCACACGTAACCAGATGGGAGAAGCTCTCGTATTCCAACAGC
TGTAGGTTGACCGTCTGAATCCTTCACTATGGCCTTTATACCTGGATGCAGATCCAACAGGACTTG
TCTACATCTACCACAAGGTGACAAGATCCCGCGATTCTCGTTACCAATAGCAACGATACAAGTCAG
ATTCCCTGCGGCGGCGGCAGCGGCGGTTCCCAGTACTACGAGTTCTGCGCAGGGGCCACCCGTAAA
ATGATACACATTTACCCCAGTAAAAATCCGCCCATCGGATGACAAGGCTGCACTAGCCACGGAATA
GTCGTCAGATATAGGTATGGAATTGATTGTGGCGGTAGCCCTCTCTATCAGCGTGGACTCCTCCTG
AGAGAGGGGTTTTGCCATGGTGGCGGCTTAAGGGTTCGATCCTCTAGAGTCCGGAGGCTGGATCGG
TCCCGGTGTCTTCTATGGAGGTCAAAACAGCGTGGATGGCGTCTCCAGGCGATCTGACGGTTCACT
AAACGAGCTCTGCTTATATAGACCTCCCACCGTACACGCCTACCGCCCATTTGCGTCAATGGGGCG
GAGTTGTTACGACATTTTGGAAAGTCCCGTTGATTTTGGTGCCAAAACAAACTCCCATTGACGTCA
ATGGGGTGGAGACTTGGAAATCCCCGTGAGTCAAACCGCTATCCACGCCCATTGATGTACTGCCAA
AACCGCATCACCATGGTAATAGCGATGACTAATACGTAGATGTACTGCCAAGTAGGAAAGTCCCAT
AAGGTCATGTACTGGGCATAATGCCAGGCGGGCCATTTACCGTCATTGACGTCAATAGGGGGCGTA
CTTGGCATATGATACACTTGATGTACTGCCAAGTGGGCAGTTTACCGTAAATACTCCACCCATTGA
CGTCAATGGAAAGTCCCTATTGGCGTTACTATGGGAACATACGTCATTATTGACGTCAATGGGCGG
GGGTCGTTGGGCGGTCAGCCAGGCGGGCCATTTACCGTAAGTTATGTAACGCGGAACTCCATATAT
GGGCTATGAACTAATGACCCCGTAATTGATTACTATTAATAACTAGTCAATAATCAATGTCATTGG
GAAAAGCGCTCCCCTACCCATAACTTCGTATAATGTATGCTATACGAAGTTATTTTGCAGTTTTAA
AATTATGTTTTAAAATGGACTATCATATGCTTACCGTAACTTGAAAGTATTTCGATTTCTTGGCTT
TATATATCTTGTGGAAAGGACGAAACACCGGGCACTCTTCCGTGATCTGGTGGATAAATTCGCAAG
GGTATCATGGCGGACGACCGGGATTCGAACCCCGGATCCGGCCGTCCGCCGTGATCCATGCGGTTA
CCGCCCGCGTGTCGAACCCAGGTGTGCGACGTCAGACAACGGGGGAGCGCTCCTTTTTGGGCCCAT
SEQ ID NO: 29 (STXC0126)
TGGTATGGCTTTTTCCCCGTATCCCCCCAGGTGTCTGCAGGCTCAAAGAGCAGCGAGAAGCGTTCA
GAGGAAAGCGATCCCGTGCCACCTTCCCCGTGCCCGGGCTGTCCCCGCACGCTGCCGGCTCGGGGA
TGCGGGGGGAGCGCCGGACCGGAGCGGAGCCCCGGGCGGCTCGCTGCTGCCCCCTAGCGGGGGAGG
GACGTAATTACATCCCTGGGGGCTTTGGGGGGGGGCTGTCCCTGATATCTATAACAAGAAAATATA
TATATAATAAGTTATCACGTAAGTAGAACATGAAATAACAATATAATTATCGTATGAGTTAAATCT
TAAAAGTCACGTAAAAGATAATCATGCGTCATTTTGACTCACGCGGTCGTTATAGTTCAAAATCAG
TGACACTTACCGCATTGACAAGCACGCCTCACGGGAGCTCCAAGCGGCGACTGAGATGTCCTAAAT
GCACAGCGACGGATTCGCGCTATTTAGAAAGAGAGAGCAATATTTCAAGAATGCATGCGTCAATTT
TACGCAGACTATCTTTCTAGGGTTAATCTAGCTGCATCAGGATCATATCGTCGGGTCTTTTTTCCG
GCTCAGTCATCGCCCAAGCTGGCGCTATCTGGGCATCGGGGAGGAAGAAGCCCGTGCCTTTTCCCG
CGAGGTTGAAGCGGCATGGAAAGAGTTTGCCGAGGATGACTGCTGCTGCATTGACGTTGAGCGAAA
ACGCACGTTTACCATGATGATTCGGGAAGGTGTGGCCATGCACGCCTTTAACGGTGAACTGTTCGT
TCAGGCCACCTGGGATACCAGTTCGTCGCGGCTTTTCCGGACACAGTTCCGGATGGTCAGCCCGAA
GCGCATCAGCAACCCGAACAATACCGGCGACAGCCGGAACTGCCGTGCCGGTGTGCAGATTAATGA
CAGCGGTGCGGCGCTGGGATATTACGTCAGCGAGGACGGGTATCCTGGCTGGATGCCGCAGAAATG
GACATGGATACCCCGTGAGTTACCCGGCGGGCGCGCTTGGCGTAATCATGGTCATAGCTGTTTCCT
GTGTGAAATTGTTATCCGCTCACAATTCCACACAACATACGAGCCGGAAGCATAAAGTGTAAAGCC
TGGGGTGCCTAATGAGTGAGCTAACTCACATTAATTGCGTTGCGCTCACTGCCCGCTTTCCAGTCG
GGAAACCTGTCGTGCCAGCTGCATTAATGAATCGGCCAACGCGCGGGGAGAGGCGGTTTGCGTATT
GGGCGCTCTTCCGCTTCCTCGCTCACTGACTCGCTGCGCTCGGTCGTTCGGCTGCGGCGAGCGGTA
TCAGCTCACTCAAAGGCGGTAATACGGTTATCCACAGAATCAGGGGATAACGCAGGAAAGAACATG
TGAGCAAAAGGCCAGCAAAAGGCCAGGAACCGTAAAAAGGCCGCGTTGCTGGCGTTTTTCCATAGG
CTCCGCCCCCCTGACGAGCATCACAAAAATCGACGCTCAAGTCAGAGGTGGCGAAACCCGACAGGA
CTATAAAGATACCAGGCGTTTCCCCCTGGAAGCTCCCTCGTGCGCTCTCCTGTTCCGACCCTGCCG
CTTACCGGATACCTGTCCGCCTTTCTCCCTTCGGGAAGCGTGGCGCTTTCTCATAGCTCACGCTGT
AGGTATCTCAGTTCGGTGTAGGTCGTTCGCTCCAAGCTGGGCTGTGTGCACGAACCCCCCGTTCAG
CCCGACCGCTGCGCCTTATCCGGTAACTATCGTCTTGAGTCCAACCCGGTAAGACACGACTTATCG
CCACTGGCAGCAGCCACTGGTAACAGGATTAGCAGAGCGAGGTATGTAGGCGGTGCTACAGAGTTC
TTGAAGTGGTGGCCTAACTACGGCTACACTAGAAGGACAGTATTTGGTATCTGCGCTCTGCTGAAG
CCAGTTACCTTCGGAAAAAGAGTTGGTAGCTCTTGATCCGGCAAACAAACCACCGCTGGTAGCGGT
GGTTTTTTTGTTTGCAAGCAGCAGATTACGCGCAGAAAAAAAGGATCTCAAGAAGATCCTTTGATC
TTTTCTACGGGGTCTGACGCTCAGTGGAACGAAAACTCACGTTAAGGGATTTTGGTCATGAGATTA
TCAAAAAGGATCTTCACCTAGATCCTTTTAAATTAAAAATGAAGTTTTAAATCAATCTAAAGTATA
TATGAGTAAACTTGGTCTGACAGTTACCAATGCTTAATCAGTGAGGCACCTATCTCAGCGATCTGT
CTATTTCGTTCATCCATAGTTGCCTGACTCCCCGTCGTGTAGATAACTACGATACGGGAGGGCTTA
CCATCTGGCCCCAGTGCTGCAATGATACCGCGAGACCCACGCTCACCGGCTCCAGATTTATCAGCA
ATAAACCAGCCAGCCGGAAGGGCCGAGCGCAGAAGTGGTCCTGCAACTTTATCCGCCTCCATCCAG
TCTATTAATTGTTGCCGGGAAGCTAGAGTAAGTAGTTCGCCAGTTAATAGTTTGCGCAACGTTGTT
GCCATTGCTACAGGCATCGTGGTGTCACGCTCGTCGTTTGGTATGGCTTCATTCAGCTCCGGTTCC
CAACGATCAAGGCGAGTTACATGATCCCCCATGTTGTGCAAAAAAGCGGTTAGCTCCTTCGGTCCT
CCGATCGTTGTCAGAAGTAAGTTGGCCGCAGTGTTATCACTCATGGTTATGGCAGCACTGCATAAT
TCTCTTACTGTCATGCCATCCGTAAGATGCTTTTCTGTGACTGGTGAGTACTCAACCAAGTCATTC
TGAGAATAGTGTATGCGGCGACCGAGTTGCTCTTGCCCGGCGTCAATACGGGATAATACCGCGCCA
CATAGCAGAACTTTAAAAGTGCTCATCATTGGAAAACGTTCTTCGGGGCGAAAACTCTCAAGGATC
TTACCGCTGTTGAGATCCAGTTCGATGTAACCCACTCGTGCACCCAACTGATCTTCAGCATCTTTT
ACTTTCACCAGCGTTTCTGGGTGAGCAAAAACAGGAAGGCAAAATGCCGCAAAAAAGGGAATAAGG
GCGACACGGAAATGTTGAATACTCATACTCTTCCTTTTTCAATATTATTGAAGCATTTATCAGGGT
TATTGTCTCATGAGCGGATACATATTTGAATGTATTTAGAAAAATAAACAAATAGGGGTTCCGCGC
ACATTTCCCCGAAAAGTGCCACCTAAATTGTAAGCGTTAATATTTTGTTAAAATTCGCGTTAAATT
TTTGTTAAATCAGCTCATTTTTTAACCAATAGGCCGAAATCGGCAAAATCCCTTATAAATCAAAAG
AATAGACCGAGATAGGGTTGAGTGTTGTTCCAGTTTGGAACAAGAGTCCACTATTAAAGAACGTGG
ACTCCAACGTCAAAGGGCGAAAAACCGTCTATCAGGGCGATGGCCCACTACGTGAACCATCACCCT
AATCAAGTTTTTTGGGGTCGAGGTGCCGTAAAGCACTAAATCGGAACCCTAAAGGGAGCCCCCGAT
TTAGAGCTTGACGGGGAAAGCCGGCGAACGTGGCGAGAAAGGAAGGGAAGAAAGCGAAAGGAGCGG
GCGCTAGGGCGCTGGCAAGTGTAGCGGTCACGCTGCGCGTAACCACCACACCCGCCGCGCTTAATG
CGCCGCTACAGGGCGCGTCCCATTCGCCATTCAGGCTGCGCAACTGTTGGGAAGGGCGATCGGTGC
GGGCCTCTTCGCTATTACGCCAGCTGGCGAAAGGGGGATGTGCTGCAAGGCGATTAAGTTGGGTAA
CGCCAGGGTTTTCCCAGTCACGACGTTGTAAAACGACGGCCAGTGAGCGCGCCTCGTTCATTCACG
TTTTTGAACCCGTGGAGGACGGGCAGACTCGCGGTGCAAATGTGTTTTACAGCGTGATGGAGCAGA
TGAAGATGCTCGACACGCTGCAGAACACGCAGCTAGATTAACCCTAGAAAGATAATCATATTGTGA
CGTACGTTAAAGATAATCATGTGTAAAATTGACGCATGTGTTTTATCGGTCTGTATATCGAGGTTT
ATTTATTAATTTGAATAGATATTAAGTTTTATTATATTTACACTTACATACTAATAATAAATTCAA
CAAACAATTTATTTATGTTTATTTATTTATTAAAAAAAACAAAAACTCAAAATTTCTTCTATAAAG
TAACAAAACTTTTATGAGGGACAGCCCCCCCCCAAAGCCCCCAGGGATGTAATTACGTCCCTCCCC
CGCTAGGGGGCAGCAGCGAGCCGCCCGGGGCTCCGCTCCGGTCCGGCGCTCCCCCCGCATCCCCGA
GCCGGCAGCGTGCGGGGACAGCCCGGGCACGGGGAAGGTGGCACGGGATCGCTTTCCTCTGAACGC
TTCTCGCTGCTCTTTGAGCCTGCAGACACCTGGGGGGATACGGGGAAAAGGCCTCCACGGCCACTA
GTCCATAGAGCCCACCGCATCCCCAGCATGCCTGCTATTGTCTTCCCAATCCTCCCCCTTGCTGTC
CTGCCCCACCCCACCCCCTAGAATAGAATGACACCTACTCAGACAATGCGATGCAATTTCCTCATT
TTATTAGGAAAGGACAGTGGGAGTGGCACCTTCCAGGGTCAAGGAAGGCACGGGGGAGGGGCAAAC
AACAGATGGCTGGCAACTAGAAGGCACAGCTACATGGGGGTAGAGTCATAATCGTGCATCAGGATA
GGGCGGTGGTGCTGCAGCAGCGCGCGAATAAACTGCTGCCGCCGCCGCTCCGTCCTGCAGGAATAC
AACATGGCAGTGGTCTCCTCAGCGATGATTCGCACCGCCCGCAGCATGAGACGCCTTGTCCTCCGG
GCACAGCAGCGCACCCTGATCTCACTTAAATCAGCACAGTAACTGCAGCACAGCACCACAATATTG
TTCAAAATCCCACAGTGCAAGGCGCTGTATCCAAAGCTCATGGCGGGGACCACAGAACCCACGTGG
CCATCATACCACAAGCGCAGGTAGATTAAGTGGCGACCCCTCATAAACACGCTGGACATAAACATT
ACCTCTTTTGGCATGTTGTAATTCACCACCTCCCGGTACCATATAAACCTCTGATTAAACATGGCG
CCATCCACCACCATCCTAAACCAGCTGGCCAAAACCTGCCCGCCGGCTATGCACTGCAGGGAACCG
GGACTGGAACAATGACAGTGGAGAGCCCAGGACTCGTAACCATGGATCATCATGCTCGTCATGATA
TCAATGTTGGCACAACACAGGCACACGTGCATACACTTCCTCAGGATTACAAGCTCCTCCCGCGTC
AGAACCATATCCCAGGGAACAACCCATTCCTGAATCAGCGTAAATCCCACACTGCAGGGAAGACCT
CGCACGTAACTCACGTTGTGCATTGTCAAAGTGTTACATTCGGGCAGCAGCGGATGATCCTCCAGT
ATGGTAGCGCGGGTCTCTGTCTCAAAAGGAGGTAGGCGATCCCTACTGTACGGAGTGCGCCGAGAC
AACCGAGATCGTGTTGGTCGTAGTGTCATGCCAAATGGAACGCCGGACGTAGTCATGGTTGTGGCC
ATATTATCATCGTGTTTTTCAAAGGAAAACCACGTCCCCGTGGTTCGGGGGGCCTAGACGTTTTTT
TAACCTCGACTAAACACATGTAAAGCATGTGCACCGAGGCCCCAGATCAGATCCCATACAATGGGG
TACCTTCTGGGCATCCTTCAGCCCCTTGTTGAATACGCTTGAGGAGAGCCATTTGACTCTTTCCAC
AACTATCCAACTCACAACGTGGCACTGGGGTTGTGCCGCCTTTGCAGGTGTATCTTATACACGTGG
CTTTTGGCCGCAGAGGCACCTGTCGCCAGGTGGGGGGTTCCGCTGCCTGCAAAGGGTCGCTACAGA
CGTTGTTTGTCTTCAAGAAGCTTCCAGAGGAACTGCTTCCTTCACGACATTCAACAGACCTTGCAT
TCCTTTGGCGAGAGGGGAAAGACCCCTAGGAATGCTCGTCAAGAAGACAGGGCCAGGTTTCCGGGC
CCTCACATTGCCAAAAGACGGCAATATGGTGGAAAATAACATATAGACAAACGCACACCGGCCTTA
TTCCAAGCGGCTTCGGCCAGTAACGTTAGGGGGGGGGGAGGGAGAGGGGCTTAAAAATCAAAGGGG
TTCTGCCGCGCATCACTATGCGCCACTGGCAGGGACACGTTGCGATACTGGTGTTTAGTGCTCCAC
TTAAACTCAGGCACAACCATCCGCGGCAGCTCGGTGAAGTTTTCACTCCACAGGCTGCGCACCATC
ACCAACGCGTTTAGCAGGTCGGGCGCCGATATCTTGAAGTCGCAGTTGGGGCCTCCGCCCTGCGCG
CGCGAGTTGCGATACACAGGGTTGCAGCACTGGAACACTATCAGCGCCGGGTGGTGCACGCTGGCC
AGCACGCTCTTGTCGGAGATCAGATCCGCGTCCAGGTCCTCCGCGTTGCTCAGGGCGAACGGAGTC
AACTTTGGTAGCTGCCTTCCCAAAAAGGGTGCATGCCCAGGCTTTGAGTTGCACTCGCACCGTAGT
GGCATCAGAAGGTGACCGTGCCCGGTCTGGGCGTTAGGATACAGCGCCTGCATGAAAGCCTTGATC
TGCTTAAAAGCCACCTGAGCCTTTGCGCCTTCAGAGAAGAACATGCCGCAAGACTTGCCGGAAAAC
TGATTGGCCGGACAGGCCGCGTCATGCACGCAGCACCTTGCGTCGGTGTTGGAGATCTGCACCACA
TTTCGGCCCCACCGGTTCTTCACGATCTTGGCCTTGCTAGACTGCTCCTTCAGCGCGCGCTGCCCG
TTTTCGCTCGTCACATCCATTTCAATCACGTGCTCCTTATTTATCATAATGCTCCCGTGTAGACAC
TTAAGCTCGCCTTCGATCTCAGCGCAGCGGTGCAGCCACAACGCGCAGCCCGTGGGCTCGTGGTGC
TTGTAGGTTACCTCTGCAAACGACTGCAGGTACGCCTGCAGGAATCGCCCCATCATCGTCACAAAG
GTCTTGTTGCTGGTGAAGGTCAGCTGCAACCCGCGGTGCTCCTCGTTTAGCCAGGTCTTGCATACG
GCCGCCAGAGCTTCCACTTGGTCAGGCAGTAGCTTGAAGTTTGCCTTTAGATCGTTATCCACGTGG
TACTTGTCCATCAACGCGCGCGCAGCCTCCATGCCCTTCTCCCACGCAGACACGATCGGCAGGCTC
AGCGGGTTTATCACCGTGCTTTCACTTTCCGCTTCACTGGACTCTTCCTTTTCCTCTTGCGTCCGC
ATACCCCGCGCCACTGGGTCGTCTTCATTCAGCCGCCGCACCGTGCGCTTACCTCCCTTGCCGTGC
TTGATTAGCACCGGTGGGTTGCTGAAACCCACCATTTGTAGCGCCACATCTTCTCTTTCTTCCTCG
CTGTCCACGATCACCTCTGGGGATGGCGGGCGCTCGGGCTTGGGAGAGGGGCGCTTCTTTTTCTTT
TTGGACGCAATGGCCAAATCCGCCGTCGAGGTCGATGGCCGCGGGCTGGGTGTGCGCGGCACCAGC
GCATCTTGTGACGAGTCTTCTTCGTCCTCGGACTCGAGACGCCGCCTCAGCCGCTTTTTTGGGGGC
GCGCGCTTGTCGTCATCGTCTTTGTAGTCGGGAGGCGGCGGCGACGGCGACGGGGACGACACGTCC
TCCATGGTTGGTGGACGTCGCGCCGCACCGCGTCCGCGCTCGGGGGTGGTTTCGCGCTGCTCCTCT
TCCCGACTGGCCATGGTGGCCGAGGATAACTTCGTATATGGTTTCTTATACGAAGTTATGATCCAG
ACATGATAAGATACATTGATGAGTTTGGACAAACCACAACTAGAATGCAGTGAAAAAAATGCTTTA
TTTGTGAAATTTGTGATGCTATTGCTTTATTTGTAACCATTATAAGCTGCAATAAACAAGTTAACA
ACAACAATTGCATTCATTTTATGTTTCAGGTTCAGGGGGAGGTGTGGGAGGTTTTTTAAAGCAAGT
AAAACCTCTACAAATGTGGTATGGCTGATTATGATCCTCTAGAGTCGCAGATCTGCTACGTATCAA
GCTGTGGCAGGGAAACCCTCTGCCTCCCCCGTGATGTAATACTTTTGCAAGGAATGCGATGAAGTA
GAGCCCGCAGTGGCCAAGTGGCTTTGGTCCGTCTCCTCCACGGATGCCCCTCCACGGCTAGTGGGC
GCATGTAGGCGGTGGGCGTCCGCCGCCTCCAGCAGCAGGTCATAGAGGGGCACCACGTTCTTGCAC
TTCATGCTGTACAGATGCTCCATGCCTTTGTTACTCATGTGTCGGATGTGGGAGAGGATGAGGAGG
AGCTGGGCCAGCCGCTGGTGCTGCTGCTGCAGGGTCAGGCCTGCCTTGGCCATCAGGTGGATCAAA
GTGTCTGTGATCTTGTCCAGGACTCGGTGGATATGGTCCTTCTCTTCCAGAGACTTCAGGGTGCTG
GACAGAAATGTGTACACTCCAGAATTAAGCAAAATAATAGATTTGAGGCACACAAACTCCTCTCCC
TGCAGATTCATCATGCGGAACCGAGATGATGTAGCCAGCAGCATGTCGAAGATCTCCACCATGCCC
TCTACACATTTTCCCTGGTTCCTGTCCAAGAGCAAGTTAGGAGCAAACAGTAGCTTCACTGGGTGC
TCCATGGAGCGCCAGACGAGACCAATCATCAGGATCTCTAGCCAGGCACATTCTAGAAGGTGGACC
TGATCATGGAGGGTCAAATCCACAAAGCCTGGCACCCTCTTCGCCCAGTTGATCATGTGAACCAGC
TCCCTGTCTGCCAGGTTGGTCAGTAAGCCCATCATCGAAGCTTCACTGAAGGGTCTGGTAGGATCA
TACTCGGAATAGAGTATGGGGGGCTCAGCATCCAACAAGGCACTGACCATCTGGTCGGCCGTCAGG
GACAAGGCCAGGCTGTTCTTCTTAGAGCGTTTGATCATGAGCGGGCTTGGCCAAAGGTTGGCAGCT
CTCATGTCTCCAGCAGATGGCTCGAGATCGCCATCTTCCAGCAGGCGCACCATTGCCCCTGTTTCA
CTATCCAGGTTACGGATATAGTTCATGACAATATTTACATTGGTCCAGCCACCAGCTTGCATGATC
TCCGGTATTGAAACTCCAGCGCGGGCCATATCTCGCGCGGCTCCGACACGGGCACTGTGTCCAGAC
CAGGCCAGGTATCTCTGACCAGAGTCATCCTAAAATACACAAACAATTAGAATCAGTAGTTTAACA
CATTATACACTTAAAAATTTTATATTTACCTTAGCGCCGTAAATCAATCGATGAGTTGCTTCAAAA
ATCCCTTCCAGGGCGCGAGTTGATAGCTGGCTGGTGGCAGATGGCGCGGCAACACCATTTTTTCTG
ACCCGGCAAAACAGGTAGTTATTCGGATCATCAGCTACACCAGAGACGGAAATCCATCGCTCGACC
AGTTTAGTGACTCCCAGGCTAAGTGCCTTCTCTACACCTGCGGTGCTAACCAGCGTTTTCGTTCTG
CCAATATGGATTAACATTCTCCCACCGTCAGTACGTGAGATATCTTTAACCCTGATCCTGGCAATT
TCGGCTATACGTAACAGGGTGTTATAAGCAATCCCCAGAAATGCCAGATTACGTATATCCTGGCAG
CGATCGCTATTTTCCATGAGTGAACGGACTTGGTCGAAATCAGTGCGTTCGAACGCTAGAGCCTGT
TTTGCACGTTCACCGGCATCAACGTTTTCTTTTCGGATCCGCCGCATAACCAGTGAAACAGCATTG
CTGTCACTTGGTCGTGGCAGCCCGGACCGACGATGAAGCATGTTTAGCTGGCCCAAATGTTGCTGG
ATAGTTTTTACTGCCAGACCGCGCGCTTGAAGATATAGAAGATAATCGCGAACATCTTCAGGTTCT
GCGGGAAACCATTTCCGGTTATTCAACTTGCACCATGCCGCCCACGACCGGCAAACGGACAGAAGC
ATTTTCCAGGTATGCTCAGAAAACGCCTGGCGATCCCTGAACATGTCCATCAGGTTCTTGCGAACC
TCATCACTCGTTGCATCGACCGGTAATGCAGGCAAATTTTGGTGTACGGTCAGTAAATTGGACATG
GTGGCTACGTAATAACTTCGTATATGGTTTCTTATACGAAGTTATGCGGCCGCTTTACGAGGGTAG
GAAGTGGTACGGAAAGTTGGTATAAGACAAAAGTGTTGTGGAATTGCTCCAGGCGATCTGACGGTT
CACTAAACGAGCTCTGCTTTTATAGGCGCCCACCGTACACGCCTAAAGCTTATACGTTCTCTATCA
CTGATAGGGAGTAAACTGGATATACGTTCTCTATCACTGATAGGGAGTAAACTGTAGATACGTTCT
CTATCACTGATAGGGAGTAAACTGGTCATACGTTCTCTATCACTGATAGGGAGTAAACTCCTTATA
CGTTCTCTATCACTGATAGGGAGTAAAGTCTGCATACGTTCTCTATCACTGATAGGGAGTAAACTC
TTCATACGTTCTCTATCACTGATAGGGAGTAAACTCGCGGCCGCAGAGAAATGTTCTGGCACCTGC
ACTTGCACTGGGGACAGCCTATTTTGCTAGTTTGTTTTGTTTCGTTTTGTTTTGATGGAGAGCGTA
TGTTAGTACTATCGATTCACACAAAAAACCAACACACAGATGTAATGAAAATAAAGATATTTTATT
GGATCTGCGATCGCTCCGGTGCCCGTCAGTGGGCAGAGCGCACATCGCCCACAGTCCCCGAGAAGT
TGGGGGGAGGGGTCGGCAATTGAACGGGTGCCTAGAGAAGGTGGCGCGGGGTAAACTGGGAAAGTG
ATGTCGTGTACTGGCTCCGCCTTTTTCCCGAGGGTGGGGGAGAACCGTATGTAAGTGCAGTAGTCG
CCGTGAACGTTCTTTTTCGCAACGGGTTTGCCGCCAGAACACAGCTGAAGCTTCGAGGGGCTCGCA
TCTCTCCTTCACGCGCCCGCCGCCCTACCTGAGGCCGCCATCCACGCCGGTTGAGTCGCGTTCTGC
CGCCTCCCGCCTGTGGTGCCTCCTGAACTGCGTCCGCCGTCTAGGTAAGTTTAAAGCTCAGGTCGA
GACCGGGCCTTTGTCCGGCGCTCCCTTGGAGCCTACCTAGACTCAGCCGGCTCTCCACGCTTTGCC
TGACCCTGCTTGCTCAACTCTACGTCTTTGTTTCGTTTTCTGTTCTGCGCCGTTACAGATCCAAGC
TGTGACCGGCGCCTACGCTAGCGGATCCGCCGCCACCATGTCTAGACTGGACAAGAGCAAAGTCAT
AAACTCTGCTCTGGAATTACTCAATGGAGTCGGTATCGAAGGCCTGACGACAAGGAAACTCGCTCA
AAAGCTGGGAGTTGAGCAGCCTACCCTGTACTGGCACGTGAAGAACAAGCGGGCCCTGCTCGATGC
CCTGCCAATCGAGATGCTGGACAGGCATCATACCCACTCCTGCCCCCTGGAAGGCGAGTCATGGCA
AGACTTTCTGCGGAACAACGCCAAGTCATACCGCTGTGCTCTTCTCTCACATCGCGACGGGGCTAA
AGTGCATCTCGGCACCCGCCCAACAGAGAAACAGTACGAAACCCTGGAAAATCAGCTCGCGTTCCT
GTGTCAGCAAGGCTTCTCCCTGGAGAACGCACTGTACGCTCTGTCCGCCGTGGGCCACTTTACACT
GGGCTGCGTATTGGAGGAACAGGAGCATCAAGTAGCAAAAGAGGAAAGAGAGACACCTACCACCGA
TTCTATGCCCCCACTTCTGAAACAAGCAATTGAGCTGTTCGACCGGCAGGGAGCCGAACCTGCCTT
CCTTTTCGGCCTGGAACTAATCATATGTGGCCTGGAGAAACAGCTAAAGTGCGAAAGCGGCGGGCC
GACCGACGCCCTTGACGATTTTGACTTAGACATGCTCCCAGCCGATGCCCTTGACGACTTTGACCT
TGATATGCTGCCTGCTGACGCTCTTGACGATTTTGACCTTGACATGCTCCCCGGGTGAACCGGTCG
CTGATCAGCCTCGACTGTGCCTTCTAGTTGCCAGCCATCTGTTGTTTGCCCCTCCCCCGTGCCTTC
CTTGACCCTGGAAGGTGCCACTCCCACTGTCCTTTCCTAATAAAATGAGGAAATTGCATCGCATTG
TCTGAGTAGGTGTCATTCTATTCTGGGGGGTGGGGTGGGGCAGGACAGCAAGGGGGAGGATTGGGA
AGACAATAGCAGGCATGCTGGGGATGCGGTGGGCTCTATGGCTTCTGAGGCGGAAAGAACCAGCTG
GGGCTCGACTAGAGCTTGCGGAACCCTTAGAGGGCCTATTTCCCATGATTCCTTCATATTTGCATA
TACGATACAAGGCTGTTAGAGAGATAATTAGAATTAATTTGACTGTAAACACAAAGATATTAGTAC
AAAATAATAACTTCGTATAATGTATGCTATACGAAGTTATCAGACATGATAAGATACATTGATGAG
TTTGGACAAACCACAACTAGAATGCAGTGAAAAAAATGCTTTATTTGTGAAATTTGTGATGCTATT
GCTTTATTTGTAACCATTATAAGCTGCAATAAACAAGGTACCTCAAGCGCCGGGTTTTCGCGTCAT
GCACCACGTCCGTGGTCTAGAACTAGTATTATGCCCAGTACATGACCTTATGGGACTTTCCTACTT
GGCAGTACATCTACGTATTAGTCATCGCTATTACCATGGTGATGCGGTTTTGGCAGTACATCAATG
GGCGTGGATAGCGGTTTGACTCACGGGGATTTCCAAGTCTCCACCCCATTGACGTCAATGGGAGTT
TGTTTTGGCACCAAAATCAACGGGACTTTCCAAAATGTCGTAACAACTCCGCCCCATTGACGCAAA
TGGGCGGTAGGCGTGTACGGTGGGAGGTTTATATAAGCAGAGCTCGTTTAGTGAACCGTCAGATCG
CCTGGAGACGCCATCCACGCTGTTTTGACCTCCATAGAAGAGAATTCGCCGCCACCATGAAAACAT
TTAACATTTCTCAACAGGATCTAGAATTAGTAGAAGTAGCGACAGAGAAGATTACAATGCTTTATG
AGGATAATAAACATCATGTGGGAGCGGCAATTCGTACGAAAACAGGAGAAATCATTTCGGCAGTAC
ATATTGAAGCGTATATAGGACGAGTAACTGTTTGTGCAGAAGCCATTGCGATTGGTAGTGCAGTTT
CGAATGGACAAAAGGATTTTGACACGATTGTAGCTGTTAGACACCCTTATTCTGACGAAGTAGATA
GAAGTATTCGAGTGGTAAGTCCTTGTGGTATGTGTAGGGAGTTGATTTCAGACTATGCACCAGATT
GTTTTGTGTTAATAGAAATGAATGGCAAGTTAGTCAAAACTACGATTGAAGAACTCATTCCACTCA
AATATACCCGAAATTAAACCGGTCGCTGATCAGCCTCGACTGTGCCTTCTAGTTGCCAGCCATCTG
TTGTTTGCCCCTCCCCCGTGCCTTCCTTGACCCTGGAAGGTGCCACTCCCACTGTCCTTTCCTAAT
AAAATGAGGAAATTGCATCGCATTGTCTGAGTAGGTGTCATTCTATTCTGGGGGGTGGGGTGGGGC
AGGACAGCAAGGGGGAGGATTGGGAAGACAATAGCAGGCATGCTGGGGATGCGGTGGGCTCTATGG
CTTCTGAGGCGGAAAGAACCAGCTGGGGCTCGACTAGAGCTTGCGGAACCCTTAGGGCCCATTGGT
ATGGCTTGGGAAAAGCGCTCCCCTACCCATAACTTCGTATAATGTATGCTATACGAAGTTATTTTG
CAGTTTTAAAATTATGTTTTAAAATGGACTATCATATGCTTACCGTAACTTGAAAGTATTTCGATT
TCTTGGCTTTATATATCTTGTGGAAAGGACGAAACACCGGGCACTCTTCCGTGATCTGGTGGATAA
ATTCGCAAGGGTATCATGGCGGACGACCGGGATTCGAACCCCGGATCCGGCCGTCCGCCGTGATCC
ATGCGGTTACCGCCCGCGTGTCGAACCCAGGTGTGCGACGTCAGACAACGGGGGAGCGCTCCTTTT
TGGGCCCAT
SEQ ID NO: 30 (STXC0123)
TGGTATGGCTTTTTCCCCGTATCCCCCCAGGTGTCTGCAGGCTCAAAGAGCAGCGAGAAGCGTTCA
GAGGAAAGCGATCCCGTGCCACCTTCCCCGTGCCCGGGCTGTCCCCGCACGCTGCCGGCTCGGGGA
TGCGGGGGGAGCGCCGGACCGGAGCGGAGCCCCGGGCGGCTCGCTGCTGCCCCCTAGCGGGGGAGG
GACGTAATTACATCCCTGGGGGCTTTGGGGGGGGGCTGTCCCTGATATCTATAACAAGAAAATATA
TATATAATAAGTTATCACGTAAGTAGAACATGAAATAACAATATAATTATCGTATGAGTTAAATCT
TAAAAGTCACGTAAAAGATAATCATGCGTCATTTTGACTCACGCGGTCGTTATAGTTCAAAATCAG
TGACACTTACCGCATTGACAAGCACGCCTCACGGGAGCTCCAAGCGGCGACTGAGATGTCCTAAAT
GCACAGCGACGGATTCGCGCTATTTAGAAAGAGAGAGCAATATTTCAAGAATGCATGCGTCAATTT
TACGCAGACTATCTTTCTAGGGTTAATCTAGCTGCATCAGGATCATATCGTCGGGTCTTTTTTCCG
GCTCAGTCATCGCCCAAGCTGGCGCTATCTGGGCATCGGGGAGGAAGAAGCCCGTGCCTTTTCCCG
CGAGGTTGAAGCGGCATGGAAAGAGTTTGCCGAGGATGACTGCTGCTGCATTGACGTTGAGCGAAA
ACGCACGTTTACCATGATGATTCGGGAAGGTGTGGCCATGCACGCCTTTAACGGTGAACTGTTCGT
TCAGGCCACCTGGGATACCAGTTCGTCGCGGCTTTTCCGGACACAGTTCCGGATGGTCAGCCCGAA
GCGCATCAGCAACCCGAACAATACCGGCGACAGCCGGAACTGCCGTGCCGGTGTGCAGATTAATGA
CAGCGGTGCGGCGCTGGGATATTACGTCAGCGAGGACGGGTATCCTGGCTGGATGCCGCAGAAATG
GACATGGATACCCCGTGAGTTACCCGGCGGGCGCGCTTGGCGTAATCATGGTCATAGCTGTTTCCT
GTGTGAAATTGTTATCCGCTCACAATTCCACACAACATACGAGCCGGAAGCATAAAGTGTAAAGCC
TGGGGTGCCTAATGAGTGAGCTAACTCACATTAATTGCGTTGCGCTCACTGCCCGCTTTCCAGTCG
GGAAACCTGTCGTGCCAGCTGCATTAATGAATCGGCCAACGCGCGGGGAGAGGCGGTTTGCGTATT
GGGCGCTCTTCCGCTTCCTCGCTCACTGACTCGCTGCGCTCGGTCGTTCGGCTGCGGCGAGCGGTA
TCAGCTCACTCAAAGGCGGTAATACGGTTATCCACAGAATCAGGGGATAACGCAGGAAAGAACATG
TGAGCAAAAGGCCAGCAAAAGGCCAGGAACCGTAAAAAGGCCGCGTTGCTGGCGTTTTTCCATAGG
CTCCGCCCCCCTGACGAGCATCACAAAAATCGACGCTCAAGTCAGAGGTGGCGAAACCCGACAGGA
CTATAAAGATACCAGGCGTTTCCCCCTGGAAGCTCCCTCGTGCGCTCTCCTGTTCCGACCCTGCCG
CTTACCGGATACCTGTCCGCCTTTCTCCCTTCGGGAAGCGTGGCGCTTTCTCATAGCTCACGCTGT
AGGTATCTCAGTTCGGTGTAGGTCGTTCGCTCCAAGCTGGGCTGTGTGCACGAACCCCCCGTTCAG
CCCGACCGCTGCGCCTTATCCGGTAACTATCGTCTTGAGTCCAACCCGGTAAGACACGACTTATCG
CCACTGGCAGCAGCCACTGGTAACAGGATTAGCAGAGCGAGGTATGTAGGCGGTGCTACAGAGTTC
TTGAAGTGGTGGCCTAACTACGGCTACACTAGAAGGACAGTATTTGGTATCTGCGCTCTGCTGAAG
CCAGTTACCTTCGGAAAAAGAGTTGGTAGCTCTTGATCCGGCAAACAAACCACCGCTGGTAGCGGT
GGTTTTTTTGTTTGCAAGCAGCAGATTACGCGCAGAAAAAAAGGATCTCAAGAAGATCCTTTGATC
TTTTCTACGGGGTCTGACGCTCAGTGGAACGAAAACTCACGTTAAGGGATTTTGGTCATGAGATTA
TCAAAAAGGATCTTCACCTAGATCCTTTTAAATTAAAAATGAAGTTTTAAATCAATCTAAAGTATA
TATGAGTAAACTTGGTCTGACAGTTACCAATGCTTAATCAGTGAGGCACCTATCTCAGCGATCTGT
CTATTTCGTTCATCCATAGTTGCCTGACTCCCCGTCGTGTAGATAACTACGATACGGGAGGGCTTA
CCATCTGGCCCCAGTGCTGCAATGATACCGCGAGACCCACGCTCACCGGCTCCAGATTTATCAGCA
ATAAACCAGCCAGCCGGAAGGGCCGAGCGCAGAAGTGGTCCTGCAACTTTATCCGCCTCCATCCAG
TCTATTAATTGTTGCCGGGAAGCTAGAGTAAGTAGTTCGCCAGTTAATAGTTTGCGCAACGTTGTT
GCCATTGCTACAGGCATCGTGGTGTCACGCTCGTCGTTTGGTATGGCTTCATTCAGCTCCGGTTCC
CAACGATCAAGGCGAGTTACATGATCCCCCATGTTGTGCAAAAAAGCGGTTAGCTCCTTCGGTCCT
CCGATCGTTGTCAGAAGTAAGTTGGCCGCAGTGTTATCACTCATGGTTATGGCAGCACTGCATAAT
TCTCTTACTGTCATGCCATCCGTAAGATGCTTTTCTGTGACTGGTGAGTACTCAACCAAGTCATTC
TGAGAATAGTGTATGCGGCGACCGAGTTGCTCTTGCCCGGCGTCAATACGGGATAATACCGCGCCA
CATAGCAGAACTTTAAAAGTGCTCATCATTGGAAAACGTTCTTCGGGGCGAAAACTCTCAAGGATC
TTACCGCTGTTGAGATCCAGTTCGATGTAACCCACTCGTGCACCCAACTGATCTTCAGCATCTTTT
ACTTTCACCAGCGTTTCTGGGTGAGCAAAAACAGGAAGGCAAAATGCCGCAAAAAAGGGAATAAGG
GCGACACGGAAATGTTGAATACTCATACTCTTCCTTTTTCAATATTATTGAAGCATTTATCAGGGT
TATTGTCTCATGAGCGGATACATATTTGAATGTATTTAGAAAAATAAACAAATAGGGGTTCCGCGC
ACATTTCCCCGAAAAGTGCCACCTAAATTGTAAGCGTTAATATTTTGTTAAAATTCGCGTTAAATT
TTTGTTAAATCAGCTCATTTTTTAACCAATAGGCCGAAATCGGCAAAATCCCTTATAAATCAAAAG
AATAGACCGAGATAGGGTTGAGTGTTGTTCCAGTTTGGAACAAGAGTCCACTATTAAAGAACGTGG
ACTCCAACGTCAAAGGGCGAAAAACCGTCTATCAGGGCGATGGCCCACTACGTGAACCATCACCCT
AATCAAGTTTTTTGGGGTCGAGGTGCCGTAAAGCACTAAATCGGAACCCTAAAGGGAGCCCCCGAT
TTAGAGCTTGACGGGGAAAGCCGGCGAACGTGGCGAGAAAGGAAGGGAAGAAAGCGAAAGGAGCGG
GCGCTAGGGCGCTGGCAAGTGTAGCGGTCACGCTGCGCGTAACCACCACACCCGCCGCGCTTAATG
CGCCGCTACAGGGCGCGTCCCATTCGCCATTCAGGCTGCGCAACTGTTGGGAAGGGCGATCGGTGC
GGGCCTCTTCGCTATTACGCCAGCTGGCGAAAGGGGGATGTGCTGCAAGGCGATTAAGTTGGGTAA
CGCCAGGGTTTTCCCAGTCACGACGTTGTAAAACGACGGCCAGTGAGCGCGCCTCGTTCATTCACG
TTTTTGAACCCGTGGAGGACGGGCAGACTCGCGGTGCAAATGTGTTTTACAGCGTGATGGAGCAGA
TGAAGATGCTCGACACGCTGCAGAACACGCAGCTAGATTAACCCTAGAAAGATAATCATATTGTGA
CGTACGTTAAAGATAATCATGTGTAAAATTGACGCATGTGTTTTATCGGTCTGTATATCGAGGTTT
ATTTATTAATTTGAATAGATATTAAGTTTTATTATATTTACACTTACATACTAATAATAAATTCAA
CAAACAATTTATTTATGTTTATTTATTTATTAAAAAAAACAAAAACTCAAAATTTCTTCTATAAAG
TAACAAAACTTTTATGAGGGACAGCCCCCCCCCAAAGCCCCCAGGGATGTAATTACGTCCCTCCCC
CGCTAGGGGGCAGCAGCGAGCCGCCCGGGGCTCCGCTCCGGTCCGGCGCTCCCCCCGCATCCCCGA
GCCGGCAGCGTGCGGGGACAGCCCGGGCACGGGGAAGGTGGCACGGGATCGCTTTCCTCTGAACGC
TTCTCGCTGCTCTTTGAGCCTGCAGACACCTGGGGGGATACGGGGAAAAGGCCTCCACGGCCACTA
GTCCATAGAGCCCACCGCATCCCCAGCATGCCTGCTATTGTCTTCCCAATCCTCCCCCTTGCTGTC
CTGCCCCACCCCACCCCCTAGAATAGAATGACACCTACTCAGACAATGCGATGCAATTTCCTCATT
TTATTAGGAAAGGACAGTGGGAGTGGCACCTTCCAGGGTCAAGGAAGGCACGGGGGAGGGGCAAAC
AACAGATGGCTGGCAACTAGAAGGCACAGCTACATGGGGGTAGAGTCATAATCGTGCATCAGGATA
GGGCGGTGGTGCTGCAGCAGCGCGCGAATAAACTGCTGCCGCCGCCGCTCCGTCCTGCAGGAATAC
AACATGGCAGTGGTCTCCTCAGCGATGATTCGCACCGCCCGCAGCATGAGACGCCTTGTCCTCCGG
GCACAGCAGCGCACCCTGATCTCACTTAAATCAGCACAGTAACTGCAGCACAGCACCACAATATTG
TTCAAAATCCCACAGTGCAAGGCGCTGTATCCAAAGCTCATGGCGGGGACCACAGAACCCACGTGG
CCATCATACCACAAGCGCAGGTAGATTAAGTGGCGACCCCTCATAAACACGCTGGACATAAACATT
ACCTCTTTTGGCATGTTGTAATTCACCACCTCCCGGTACCATATAAACCTCTGATTAAACATGGCG
CCATCCACCACCATCCTAAACCAGCTGGCCAAAACCTGCCCGCCGGCTATGCACTGCAGGGAACCG
GGACTGGAACAATGACAGTGGAGAGCCCAGGACTCGTAACCATGGATCATCATGCTCGTCATGATA
TCAATGTTGGCACAACACAGGCACACGTGCATACACTTCCTCAGGATTACAAGCTCCTCCCGCGTC
AGAACCATATCCCAGGGAACAACCCATTCCTGAATCAGCGTAAATCCCACACTGCAGGGAAGACCT
CGCACGTAACTCACGTTGTGCATTGTCAAAGTGTTACATTCGGGCAGCAGCGGATGATCCTCCAGT
ATGGTAGCGCGGGTCTCTGTCTCAAAAGGAGGTAGGCGATCCCTACTGTACGGAGTGCGCCGAGAC
AACCGAGATCGTGTTGGTCGTAGTGTCATGCCAAATGGAACGCCGGACGTAGTCATGGTTGTGGCC
ATATTATCATCGTGTTTTTCAAAGGAAAACCACGTCCCCGTGGTTCGGGGGGCCTAGACGTTTTTT
TAACCTCGACTAAACACATGTAAAGCATGTGCACCGAGGCCCCAGATCAGATCCCATACAATGGGG
TACCTTCTGGGCATCCTTCAGCCCCTTGTTGAATACGCTTGAGGAGAGCCATTTGACTCTTTCCAC
AACTATCCAACTCACAACGTGGCACTGGGGTTGTGCCGCCTTTGCAGGTGTATCTTATACACGTGG
CTTTTGGCCGCAGAGGCACCTGTCGCCAGGTGGGGGGTTCCGCTGCCTGCAAAGGGTCGCTACAGA
CGTTGTTTGTCTTCAAGAAGCTTCCAGAGGAACTGCTTCCTTCACGACATTCAACAGACCTTGCAT
TCCTTTGGCGAGAGGGGAAAGACCCCTAGGAATGCTCGTCAAGAAGACAGGGCCAGGTTTCCGGGC
CCTCACATTGCCAAAAGACGGCAATATGGTGGAAAATAACATATAGACAAACGCACACCGGCCTTA
TTCCAAGCGGCTTCGGCCAGTAACGTTAGGGGGGGGGGAGGGAGAGGGGCTTAAAAATCAAAGGGG
TTCTGCCGCGCATCACTATGCGCCACTGGCAGGGACACGTTGCGATACTGGTGTTTAGTGCTCCAC
TTAAACTCAGGCACAACCATCCGCGGCAGCTCGGTGAAGTTTTCACTCCACAGGCTGCGCACCATC
ACCAACGCGTTTAGCAGGTCGGGCGCCGATATCTTGAAGTCGCAGTTGGGGCCTCCGCCCTGCGCG
CGCGAGTTGCGATACACAGGGTTGCAGCACTGGAACACTATCAGCGCCGGGTGGTGCACGCTGGCC
AGCACGCTCTTGTCGGAGATCAGATCCGCGTCCAGGTCCTCCGCGTTGCTCAGGGCGAACGGAGTC
AACTTTGGTAGCTGCCTTCCCAAAAAGGGTGCATGCCCAGGCTTTGAGTTGCACTCGCACCGTAGT
GGCATCAGAAGGTGACCGTGCCCGGTCTGGGCGTTAGGATACAGCGCCTGCATGAAAGCCTTGATC
TGCTTAAAAGCCACCTGAGCCTTTGCGCCTTCAGAGAAGAACATGCCGCAAGACTTGCCGGAAAAC
TGATTGGCCGGACAGGCCGCGTCATGCACGCAGCACCTTGCGTCGGTGTTGGAGATCTGCACCACA
TTTCGGCCCCACCGGTTCTTCACGATCTTGGCCTTGCTAGACTGCTCCTTCAGCGCGCGCTGCCCG
TTTTCGCTCGTCACATCCATTTCAATCACGTGCTCCTTATTTATCATAATGCTCCCGTGTAGACAC
TTAAGCTCGCCTTCGATCTCAGCGCAGCGGTGCAGCCACAACGCGCAGCCCGTGGGCTCGTGGTGC
TTGTAGGTTACCTCTGCAAACGACTGCAGGTACGCCTGCAGGAATCGCCCCATCATCGTCACAAAG
GTCTTGTTGCTGGTGAAGGTCAGCTGCAACCCGCGGTGCTCCTCGTTTAGCCAGGTCTTGCATACG
GCCGCCAGAGCTTCCACTTGGTCAGGCAGTAGCTTGAAGTTTGCCTTTAGATCGTTATCCACGTGG
TACTTGTCCATCAACGCGCGCGCAGCCTCCATGCCCTTCTCCCACGCAGACACGATCGGCAGGCTC
AGCGGGTTTATCACCGTGCTTTCACTTTCCGCTTCACTGGACTCTTCCTTTTCCTCTTGCGTCCGC
ATACCCCGCGCCACTGGGTCGTCTTCATTCAGCCGCCGCACCGTGCGCTTACCTCCCTTGCCGTGC
TTGATTAGCACCGGTGGGTTGCTGAAACCCACCATTTGTAGCGCCACATCTTCTCTTTCTTCCTCG
CTGTCCACGATCACCTCTGGGGATGGCGGGCGCTCGGGCTTGGGAGAGGGGCGCTTCTTTTTCTTT
TTGGACGCAATGGCCAAATCCGCCGTCGAGGTCGATGGCCGCGGGCTGGGTGTGCGCGGCACCAGC
GCATCTTGTGACGAGTCTTCTTCGTCCTCGGACTCGAGACGCCGCCTCAGCCGCTTTTTTGGGGGC
GCGCGCTTGTCGTCATCGTCTTTGTAGTCGGGAGGCGGCGGCGACGGCGACGGGGACGACACGTCC
TCCATGGTTGGTGGACGTCGCGCCGCACCGCGTCCGCGCTCGGGGGTGGTTTCGCGCTGCTCCTCT
TCCCGACTGGCCATGGTGGCCGAGGATAACTTCGTATATGGTTTCTTATACGAAGTTATGATCCAG
ACATGATAAGATACATTGATGAGTTTGGACAAACCACAACTAGAATGCAGTGAAAAAAATGCTTTA
TTTGTGAAATTTGTGATGCTATTGCTTTATTTGTAACCATTATAAGCTGCAATAAACAAGTTAACA
ACAACAATTGCATTCATTTTATGTTTCAGGTTCAGGGGGAGGTGTGGGAGGTTTTTTAAAGCAAGT
AAAACCTCTACAAATGTGGTATGGCTGATTATGATCCTCTAGAGTCGCAGATCTGCTACGTATCAA
GCTGTGGCAGGGAAACCCTCTGCCTCCCCCGTGATGTAATACTTTTGCAAGGAATGCGATGAAGTA
GAGCCCGCAGTGGCCAAGTGGCTTTGGTCCGTCTCCTCCACGGATGCCCCTCCACGGCTAGTGGGC
GCATGTAGGCGGTGGGCGTCCGCCGCCTCCAGCAGCAGGTCATAGAGGGGCACCACGTTCTTGCAC
TTCATGCTGTACAGATGCTCCATGCCTTTGTTACTCATGTGTCGGATGTGGGAGAGGATGAGGAGG
AGCTGGGCCAGCCGCTGGTGCTGCTGCTGCAGGGTCAGGCCTGCCTTGGCCATCAGGTGGATCAAA
GTGTCTGTGATCTTGTCCAGGACTCGGTGGATATGGTCCTTCTCTTCCAGAGACTTCAGGGTGCTG
GACAGAAATGTGTACACTCCAGAATTAAGCAAAATAATAGATTTGAGGCACACAAACTCCTCTCCC
TGCAGATTCATCATGCGGAACCGAGATGATGTAGCCAGCAGCATGTCGAAGATCTCCACCATGCCC
TCTACACATTTTCCCTGGTTCCTGTCCAAGAGCAAGTTAGGAGCAAACAGTAGCTTCACTGGGTGC
TCCATGGAGCGCCAGACGAGACCAATCATCAGGATCTCTAGCCAGGCACATTCTAGAAGGTGGACC
TGATCATGGAGGGTCAAATCCACAAAGCCTGGCACCCTCTTCGCCCAGTTGATCATGTGAACCAGC
TCCCTGTCTGCCAGGTTGGTCAGTAAGCCCATCATCGAAGCTTCACTGAAGGGTCTGGTAGGATCA
TACTCGGAATAGAGTATGGGGGGCTCAGCATCCAACAAGGCACTGACCATCTGGTCGGCCGTCAGG
GACAAGGCCAGGCTGTTCTTCTTAGAGCGTTTGATCATGAGCGGGCTTGGCCAAAGGTTGGCAGCT
CTCATGTCTCCAGCAGATGGCTCGAGATCGCCATCTTCCAGCAGGCGCACCATTGCCCCTGTTTCA
CTATCCAGGTTACGGATATAGTTCATGACAATATTTACATTGGTCCAGCCACCAGCTTGCATGATC
TCCGGTATTGAAACTCCAGCGCGGGCCATATCTCGCGCGGCTCCGACACGGGCACTGTGTCCAGAC
CAGGCCAGGTATCTCTGACCAGAGTCATCCTAAAATACACAAACAATTAGAATCAGTAGTTTAACA
CATTATACACTTAAAAATTTTATATTTACCTTAGCGCCGTAAATCAATCGATGAGTTGCTTCAAAA
ATCCCTTCCAGGGCGCGAGTTGATAGCTGGCTGGTGGCAGATGGCGCGGCAACACCATTTTTTCTG
ACCCGGCAAAACAGGTAGTTATTCGGATCATCAGCTACACCAGAGACGGAAATCCATCGCTCGACC
AGTTTAGTGACTCCCAGGCTAAGTGCCTTCTCTACACCTGCGGTGCTAACCAGCGTTTTCGTTCTG
CCAATATGGATTAACATTCTCCCACCGTCAGTACGTGAGATATCTTTAACCCTGATCCTGGCAATT
TCGGCTATACGTAACAGGGTGTTATAAGCAATCCCCAGAAATGCCAGATTACGTATATCCTGGCAG
CGATCGCTATTTTCCATGAGTGAACGGACTTGGTCGAAATCAGTGCGTTCGAACGCTAGAGCCTGT
TTTGCACGTTCACCGGCATCAACGTTTTCTTTTCGGATCCGCCGCATAACCAGTGAAACAGCATTG
CTGTCACTTGGTCGTGGCAGCCCGGACCGACGATGAAGCATGTTTAGCTGGCCCAAATGTTGCTGG
ATAGTTTTTACTGCCAGACCGCGCGCTTGAAGATATAGAAGATAATCGCGAACATCTTCAGGTTCT
GCGGGAAACCATTTCCGGTTATTCAACTTGCACCATGCCGCCCACGACCGGCAAACGGACAGAAGC
ATTTTCCAGGTATGCTCAGAAAACGCCTGGCGATCCCTGAACATGTCCATCAGGTTCTTGCGAACC
TCATCACTCGTTGCATCGACCGGTAATGCAGGCAAATTTTGGTGTACGGTCAGTAAATTGGACATG
GTGGCTACGTAATAACTTCGTATATGGTTTCTTATACGAAGTTATGCGGCCGCTTTACGAGGGTAG
GAAGTGGTACGGAAAGTTGGTATAAGACAAAAGTGTTGTGGAATTGCTCCAGGCGATCTGACGGTT
CACTAAACGAGCTCTGCTTTTATAGGCGCCCACCGTACACGCCTAAAGCTTATACGTTCTCTATCA
CTGATAGGGAGTAAACTGGATATACGTTCTCTATCACTGATAGGGAGTAAACTGTAGATACGTTCT
CTATCACTGATAGGGAGTAAACTGGTCATACGTTCTCTATCACTGATAGGGAGTAAACTCCTTATA
CGTTCTCTATCACTGATAGGGAGTAAAGTCTGCATACGTTCTCTATCACTGATAGGGAGTAAACTC
TTCATACGTTCTCTATCACTGATAGGGAGTAAACTCGCGGCCGCAGAGAAATGTTCTGGCACCTGC
ACTTGCACTGGGGACAGCCTATTTTGCTAGTTTGTTTTGTTTCGTTTTGTTTTGATGGAGAGCGTA
TGTTAGTACTATCGATTCACACAAAAAACCAACACACAGATGTAATGAAAATAAAGATATTTTATT
GGATCTGCGATCGCTCCGGTGCCCGTCAGTGGGCAGAGCGCACATCGCCCACAGTCCCCGAGAAGT
TGGGGGGAGGGGTCGGCAATTGAACGGGTGCCTAGAGAAGGTGGCGCGGGGTAAACTGGGAAAGTG
ATGTCGTGTACTGGCTCCGCCTTTTTCCCGAGGGTGGGGGAGAACCGTATGTAAGTGCAGTAGTCG
CCGTGAACGTTCTTTTTCGCAACGGGTTTGCCGCCAGAACACAGCTGAAGCTTCGAGGGGCTCGCA
TCTCTCCTTCACGCGCCCGCCGCCCTACCTGAGGCCGCCATCCACGCCGGTTGAGTCGCGTTCTGC
CGCCTCCCGCCTGTGGTGCCTCCTGAACTGCGTCCGCCGTCTAGGTAAGTTTAAAGCTCAGGTCGA
GACCGGGCCTTTGTCCGGCGCTCCCTTGGAGCCTACCTAGACTCAGCCGGCTCTCCACGCTTTGCC
TGACCCTGCTTGCTCAACTCTACGTCTTTGTTTCGTTTTCTGTTCTGCGCCGTTACAGATCCAAGC
TGTGACCGGCGCCTACGCTAGCGGATCCGCCGCCACCATGTCTAGACTGGACAAGAGCAAAGTCAT
AAACTCTGCTCTGGAATTACTCAATGGAGTCGGTATCGAAGGCCTGACGACAAGGAAACTCGCTCA
AAAGCTGGGAGTTGAGCAGCCTACCCTGTACTGGCACGTGAAGAACAAGCGGGCCCTGCTCGATGC
CCTGCCAATCGAGATGCTGGACAGGCATCATACCCACTCCTGCCCCCTGGAAGGCGAGTCATGGCA
AGACTTTCTGCGGAACAACGCCAAGTCATACCGCTGTGCTCTTCTCTCACATCGCGACGGGGCTAA
AGTGCATCTCGGCACCCGCCCAACAGAGAAACAGTACGAAACCCTGGAAAATCAGCTCGCGTTCCT
GTGTCAGCAAGGCTTCTCCCTGGAGAACGCACTGTACGCTCTGTCCGCCGTGGGCCACTTTACACT
GGGCTGCGTATTGGAGGAACAGGAGCATCAAGTAGCAAAAGAGGAAAGAGAGACACCTACCACCGA
TTCTATGCCCCCACTTCTGAAACAAGCAATTGAGCTGTTCGACCGGCAGGGAGCCGAACCTGCCTT
CCTTTTCGGCCTGGAACTAATCATATGTGGCCTGGAGAAACAGCTAAAGTGCGAAAGCGGCGGGCC
GACCGACGCCCTTGACGATTTTGACTTAGACATGCTCCCAGCCGATGCCCTTGACGACTTTGACCT
TGATATGCTGCCTGCTGACGCTCTTGACGATTTTGACCTTGACATGCTCCCCGGGTGAACCGGTCG
CTGATCAGCCTCGACTGTGCCTTCTAGTTGCCAGCCATCTGTTGTTTGCCCCTCCCCCGTGCCTTC
CTTGACCCTGGAAGGTGCCACTCCCACTGTCCTTTCCTAATAAAATGAGGAAATTGCATCGCATTG
TCTGAGTAGGTGTCATTCTATTCTGGGGGGTGGGGTGGGGCAGGACAGCAAGGGGGAGGATTGGGA
AGACAATAGCAGGCATGCTGGGGATGCGGTGGGCTCTATGGCTTCTGAGGCGGAAAGAACCAGCTG
GGGCTCGACTAGAGCTTGCGGAACCCTTAGAGGGCCTATTTCCCATGATTCCTTCATATTTGCATA
TACGATACAAGGCTGTTAGAGAGATAATTAGAATTAATTTGACTGTAAACACAAAGATATTAGTAC
AAAATAATAACTTCGTATAATGTATGCTATACGAAGTTATCAGACATGATAAGATACATTGATGAG
TTTGGACAAACCACAACTAGAATGCAGTGAAAAAAATGCTTTATTTGTGAAATTTGTGATGCTATT
GCTTTATTTGTAACCATTATAAGCTGCAATAAACAAGGTACCTCAAGCGCCGGGTTTTCGCGTCAT
GCACCACGTCCGTGGTTCAAGCGCCGGGTTTTCGCGTCATGCACCACGTCCGTGGGCCCTCGGGTA
CTTCAACGTCAGCAGTAACTGTAAATCCGAGCCGTTCATAGAAGGGCAAATTCCTTGGCGCTGACG
TTTCAAGAAAGGCTGGCACTCCGGCTCGTTCTGCGGCTTCTACTCCGGGCAATACCACCGCGGAAC
CAAGGCCCTTTCCCTGATGATCGGGGCTAACGCCCACAGTAGCGAGGAACCAAGCTGGTTCTTTAG
GGCGGTGAGGGGCGAGGAGTCCTTCCATTTGTTGCTGAGCCGCGAGACGAGAGCCACTAAGCTCAG
CCATTCGGGGACCAATTTCTGCAAATACAGCCCCGGCCTCAACGCTCTCCGGAGTCGTCCACACTG
CCACTGCAGCCCCGTCGTCGGCGACCCAAACTTTACCGATGTCCAATCCTACCCTGGTCAAAAAAA
GTTCTTGCAATTCTGTAACCCGTTCAATATGTCTATCAGGATCAACTGTGTGGCGTGTAGCGGGAT
AATCCGCGAAAGCGGCAGCCAATGTTCTCACGGCCCTAGGGACGTCGTCTCGAGTTGCCAGTCTGA
CAGTAGGTTTATATTCTGTCATGGTGGCGGCGAATTCTCTTCTATGGAGGTCAAAACAGCGTGGAT
GGCGTCTCCAGGCGATCTGACGGTTCACTAAACGAGCTCTGCTTATATAAACCTCCCACCGTACAC
GCCTACCGCCCATTTGCGTCAATGGGGCGGAGTTGTTACGACATTTTGGAAAGTCCCGTTGATTTT
GGTGCCAAAACAAACTCCCATTGACGTCAATGGGGTGGAGACTTGGAAATCCCCGTGAGTCAAACC
GCTATCCACGCCCATTGATGTACTGCCAAAACCGCATCACCATGGTAATAGCGATGACTAATACGT
AGATGTACTGCCAAGTAGGAAAGTCCCATAAGGTCATGTACTGGGCATAATACTAGTTCTTGGGAA
AAGCGCTCCCCTACCCATAACTTCGTATAATGTATGCTATACGAAGTTATTTTGCAGTTTTAAAAT
TATGTTTTAAAATGGACTATCATATGCTTACCGTAACTTGAAAGTATTTCGATTTCTTGGCTTTAT
ATATCTTGTGGAAAGGACGAAACACCGGGCACTCTTCCGTGATCTGGTGGATAAATTCGCAAGGGT
ATCATGGCGGACGACCGGGATTCGAACCCCGGATCCGGCCGTCCGCCGTGATCCATGCGGTTACCG
CCCGCGTGTCGAACCCAGGTGTGCGACGTCAGACAACGGGGGAGCGCTCCTTTTTGGGCCCAT
SEQ ID NO: 31 (STXC0133)
actcttcctttttcaatattattgaagcatttatcagggttattgtctcatgagcggatacatatt
tgaatgtatttagaaaaataaacaaataggggttccgcgcacatttccccgaaaagtgccacctaa
attgtaagcgttaatattttgttaaaattcgcgttaaatttttgttaaatcagctcattttttaac
caataggccgaaatcggcaaaatcccttataaatcaaaagaatagaccgagatagggttgagtgtt
gttccagtttggaacaagagtccactattaaagaacgtggactccaacgtcaaagggcgaaaaacc
gtctatcagggcgatggcccactacgtgaaccatcaccctaatcaagttttttggggtcgaggtgc
cgtaaagcactaaatcggaaccctaaagggagcccccgatttagagcttgacggggaaagccggcg
aacgtggcgagaaaggaagggaagaaagcgaaaggagcgggcgctagggcgctggcaagtgtagcg
gtcacgctgcgcgtaaccaccacacccgccgcgcttaatgcgccgctacagggcgcgtcccattcg
ccattcaggctgcgcaactgttgggaagggcgatcggtgcgggcctcttcgctattacgccagctg
gcgaaagggggatgtgctgcaaggcgattaagttgggtaacgccagggttttcccagtcacgacgt
tgtaaaacgacggccagtgagcgcgcctcgttcattcacgtttttgaacccgtggaggacgggcag
actcgcggtgcaaatgtgttttacagcgtgatggagcagatgaagatgctcgacacgctgcagaac
acgcagctagattaaccctagaaagataatcatattgtgacgtacgttaaagataatcatgtgtaa
aattgacgcatgtgttttatcggtctgtatatcgaggtttatttattaatttgaatagatattaag
ttttattatatttacacttacatactaataataaattcaacaaacaatttatttatgtttatttat
ttattaaaaaaaacaaaaactcaaaatttcttctataaagtaacaaaacttttatgagggacagcc
cccccccaaagcccccagggatgtaattacgtccctcccccgctagggggcagcagcgagccgccc
ggggctccgctccggtccggcgctccccccgcatccccgagccggcagcgtgcggggacagcccgg
gcacggggaaggtggcacgggatcgctttcctctgaacgcttctcgctgctctttgagcctgcaga
cacctggggggatacggggaaaaggcctccacggccactagtccatagagcccaccgcatccccag
catgcctgctattgtcttcccaatcctcccccttgctgtcctgccccaccccaccccctagaatag
aatgacacctactcagacaatgcgatgcaatttcctcattttattaggaaaggacagtgggagtgg
caccttccagggtcaaggaaggcacgggggaggggcaaacaacagatggctggcaactagaaggca
cagctacatgggggtagagtcataatcgtgcatcaggatagggcggtggtgctgcagcagcgcgcg
aataaactgctgccgccgccgctccgtcctgcaggaatacaacatggcagtggtctcctcagcgat
gattcgcaccgcccgcagcatgagacgccttgtcctccgggcacagcagcgcaccctgatctcact
taaatcagcacagtaactgcagcacagcaccacaatattgttcaaaatcccacagtgcaaggcgct
gtatccaaagctcatggggggaccacagaacccacgtggccatcataccacaagcgcaggtagatt
aagtggcgacccctcataaacacgctggacataaacattacctcttttggcatgttgtaattcacc
acctcccggtaccatataaacctctgattaaacatggcgccatccaccaccatcctaaaccagctg
gccaaaacctgcccgccggctatgcactgcagggaaccgggactggaacaatgacagtggagagcc
caggactcgtaaccatggatcatcatgctcgtcatgatatcaatgttggcacaacacaggcacacg
tgcatacacttcctcaggattacaagctcctcccgcgtcagaaccatatcccagggaacaacccat
tcctgaatcagcgtaaatcccacactgcagggaagacctcgcacgtaactcacgttgtgcattgtc
aaagtgttacattcgggcagcagcggatgatcctccagtatggtagcgcgggtctctgtctcaaaa
ggaggtaggcgatccctactgtacggagtgcgccgagacaaccgagatcgtgttggtcgtagtgtc
atgccaaatggaacgccggacgtagtcatggttgtggccatattatcatcgtgtttttcaaaggaa
aaccacgtccccgtggttcggggggcctagacgtttttttaacctcgactaaacacatgtaaagca
tgtgcaccgaggccccagatcagatcccatacaatggggtaccttctgggcatccttcagcccctt
gttgaatacgcttgaggagagccatttgactctttccacaactatccaactcacaacgtggcactg
gggttgtgccgcctttgcaggtgtatcttatacacgtggcttttggccgcagaggcacctgtcgcc
aggtggggggttccgctgcctgcaaagggtcgctacagacgttgtttgtcttcaagaagcttccag
aggaactgcttccttcacgacattcaacagaccttgcattcctttggcgagaggggaaagacccct
aggaatgctcgtcaagaagacagggccaggtttccgggccctcacattgccaaaagacggcaatat
ggtggaaaataacatatagacaaacgcacaccggccttattccaagcggcttcggccagtaacgtt
aggggggggggagggagaggggcttaaaaatcaaaggggttctgccgcgcatcactatgcgccact
ggcagggacacgttgcgatactggtgtttagtgctccacttaaactcaggcacaaccatccgcggc
agctcggtgaagttttcactccacaggctgcgcaccatcaccaacgcgtttagcaggtcgggcgcc
gatatcttgaagtcgcagttggggcctccgccctgcgcgcgcgagttgcgatacacagggttgcag
cactggaacactatcagcgccgggtggtgcacgctggccagcacgctcttgtcggagatcagatcc
gcgtccaggtcctccgcgttgctcagggcgaacggagtcaactttggtagctgccttcccaaaaag
ggtgcatgcccaggctttgagttgcactcgcaccgtagtggcatcagaaggtgaccgtgcccggtc
tgggcgttaggatacagcgcctgcatgaaagccttgatctgcttaaaagccacctgagcctttgcg
ccttcagagaagaacatgccgcaagacttgccggaaaactgattggccggacaggccgcgtcatgc
acgcagcaccttgcgtcggtgttggagatctgcaccacatttcggccccaccggttcttcacgatc
ttggccttgctagactgctccttcagcgcgcgctgcccgttttcgctcgtcacatccatttcaatc
acgtgctccttatttatcataatgctcccgtgtagacacttaagctcgccttcgatctcagcgcag
cggtgcagccacaacgcgcagcccgtgggctcgtggtgcttgtaggttacctctgcaaacgactgc
aggtacgcctgcaggaatcgccccatcatcgtcacaaaggtcttgttgctggtgaaggtcagctgc
aacccgcggtgctcctcgtttagccaggtcttgcatacggccgccagagcttccacttggtcaggc
agtagcttgaagtttgcctttagatcgttatccacgtggtacttgtccatcaacgcgcgcgcagcc
tccatgcccttctcccacgcagacacgatcggcaggctcagcgggtttatcaccgtgctttcactt
tccgcttcactggactcttccttttcctcttgcgtccgcataccccgcgccactgggtcgtcttca
ttcagccgccgcaccgtgcgcttacctcccttgccgtgcttgattagcaccggtgggttgctgaaa
cccaccatttgtagcgccacatcttctctttcttcctcgctgtccacgatcacctctggggatggc
gggcgctcgggcttgggagaggggcgcttctttttctttttggacgcaatggccaaatccgccgtc
gaggtcgatggccgcgggctgggtgtgcgcggcaccagcgcatcttgtgacgagtcttcttcgtcc
tcggactcgagacgccgcctcagccgcttttttgggggcgcgcgcttgtcgtcatcgtctttgtag
tcgggaggcggcggcgacggcgacggggacgacacgtcctccatggttggtggacgtcgcgccgca
ccgcgtccgcgctcgggggtggtttcgcgctgctcctcttcccgactggccatggtggccgaggat
aacttcgtatatggtttcttatacgaagttatgatccagacatgataagatacattgatgagtttg
gacaaaccacaactagaatgcagtgaaaaaaatgctttatttgtgaaatttgtgatgctattgctt
tatttgtaaccattataagctgcaataaacaagttaacaacaacaattgcattcattttatgtttc
aggttcagggggaggtgtgggaggttttttaaagcaagtaaaacctctacaaatgtggtatggctg
attatgatcctctagagtcgcagatctgctacgtatcaagctgtggcagggaaaccctctgcctcc
cccgtgatgtaatacttttgcaaggaatgcgatgaagtagagcccgcagtggccaagtggctttgg
tccgtctcctccacggatgcccctccacggctagtgggcgcatgtaggcggtgggcgtccgccgcc
tccagcagcaggtcatagaggggcaccacgttcttgcacttcatgctgtacagatgctccatgcct
ttgttactcatgtgtcggatgtgggagaggatgaggaggagctgggccagccgctggtgctgctgc
tgcagggtcaggcctgccttggccatcaggtggatcaaagtgtctgtgatcttgtccaggactcgg
tggatatggtccttctcttccagagacttcagggtgctggacagaaatgtgtacactccagaatta
agcaaaataatagatttgaggcacacaaactcctctccctgcagattcatcatgcggaaccgagat
gatgtagccagcagcatgtcgaagatctccaccatgccctctacacattttccctggttcctgtcc
aagagcaagttaggagcaaacagtagcttcactgggtgctccatggagcgccagacgagaccaatc
atcaggatctctagccaggcacattctagaaggtggacctgatcatggagggtcaaatccacaaag
cctggcaccctcttcgcccagttgatcatgtgaaccagctccctgtctgccaggttggtcagtaag
cccatcatcgaagcttcactgaagggtctggtaggatcatactcggaatagagtatggggggctca
gcatccaacaaggcactgaccatctggtcggccgtcagggacaaggccaggctgttcttcttagag
cgtttgatcatgagcgggcttggccaaaggttggcagctctcatgtctccagcagatggctcgaga
tcgccatcttccagcaggcgcaccattgcccctgtttcactatccaggttacggatatagttcatg
acaatatttacattggtccagccaccagcttgcatgatctccggtattgaaactccagcgcgggcc
atatctcgcgcggctccgacacgggcactgtgtccagaccaggccaggtatctctgaccagagtca
tcctaaaatacacaaacaattagaatcagtagtttaacacattatacacttaaaaattttatattt
accttagcgccgtaaatcaatcgatgagttgcttcaaaaatcccttccagggcgcgagttgatagc
tggctggtggcagatggcgcggcaacaccattttttctgacccggcaaaacaggtagttattcgga
tcatcagctacaccagagacggaaatccatcgctcgaccagtttagtgactcccaggctaagtgcc
ttctctacacctgcggtgctaaccagcgttttcgttctgccaatatggattaacattctcccaccg
tcagtacgtgagatatctttaaccctgatcctggcaatttcggctatacgtaacagggtgttataa
gcaatccccagaaatgccagattacgtatatcctggcagcgatcgctattttccatgagtgaacgg
acttggtcgaaatcagtgcgttcgaacgctagagcctgttttgcacgttcaccggcatcaacgttt
tcttttcggatccgccgcataaccagtgaaacagcattgctgtcacttggtcgtggcagcccggac
cgacgatgaagcatgtttagctggcccaaatgttgctggatagtttttactgccagaccgcgcgct
tgaagatatagaagataatcgcgaacatcttcaggttctgcgggaaaccatttccggttattcaac
ttgcaccatgccgcccacgaccggcaaacggacagaagcattttccaggtatgctcagaaaacgcc
tggcgatccctgaacatgtccatcaggttcttgcgaacctcatcactcgttgcatcgaccggtaat
gcaggcaaattttggtgtacggtcagtaaattggacatggtggctacgtaataacttcgtatatgg
tttcttatacgaagttatgcggccgctttacgagggtaggaagtggtacggaaagttggtataaga
caaaagtgttgtggaattgctccaggcgatctgacggttcactaaacgagctctgcttttataggc
gcccaccgtacacgcctaaagcttatacgttctctatcactgatagggagtaaactggatatacgt
tctctatcactgatagggagtaaactgtagatacgttctctatcactgatagggagtaaactggtc
atacgttctctatcactgatagggagtaaactccttatacgttctctatcactgatagggagtaaa
gtctgcatacgttctctatcactgatagggagtaaactcttcatacgttctctatcactgataggg
agtaaactcgcggccgcagagaaatgttctggcacctgcacttgcactggggacagcctattttgc
tagtttgttttgtttcgttttgttttgatggagagcgtatgttagtactatcgattcacacaaaaa
accaacacacagatgtaatgaaaataaagatattttattggatctgcgatcgctccggtgcccgtc
agtgggcagagcgcacatcgcccacagtccccgagaagttggggggaggggtcggcaattgaacgg
gtgcctagagaaggtggcgcggggtaaactgggaaagtgatgtcgtgtactggctccgcctttttc
ccgaggggggggagaaccgtatgtaagtgcagtagtcgccgtgaacgttctttttcgcaacgggtt
tgccgccagaacacagctgaagcttcgaggggctcgcatctctccttcacgcgcccgccgccctac
ctgaggccgccatccacgccggttgagtcgcgttctgccgcctcccgcctgtggtgcctcctgaac
tgcgtccgccgtctaggtaagtttaaagctcaggtcgagaccgggcctttgtccggcgctcccttg
gagcctacctagactcagccggctctccacgctttgcctgaccctgcttgctcaactctacgtctt
tgtttcgttttctgttctgcgccgttacagatccaagctgtgaccggcgcctacgctagcggatcc
gccgccaccatgtctagactggacaagagcaaagtcataaactctgctctggaattactcaatgga
gtcggtatcgaaggcctgacgacaaggaaactcgctcaaaagctgggagttgagcagcctaccctg
tactggcacgtgaagaacaagcgggccctgctcgatgccctgccaatcgagatgctggacaggcat
catacccactcctgccccctggaaggcgagtcatggcaagactttctgcggaacaacgccaagtca
taccgctgtgctcttctctcacatcgcgacggggctaaagtgcatctcggcacccgcccaacagag
aaacagtacgaaaccctggaaaatcagctcgcgttcctgtgtcagcaaggcttctccctggagaac
gcactgtacgctctgtccgccgtgggccactttacactgggctgcgtattggaggaacaggagcat
caagtagcaaaagaggaaagagagacacctaccaccgattctatgcccccacttctgaaacaagca
attgagctgttcgaccggcagggagccgaacctgccttccttttcggcctggaactaatcatatgt
ggcctggagaaacagctaaagtgcgaaagcggcgggccgaccgacgcccttgacgattttgactta
gacatgctcccagccgatgcccttgacgactttgaccttgatatgctgcctgctgacgctcttgac
gattttgaccttgacatgctccccgggtgaaccggtcgctgatcagcctcgactgtgccttctagt
tgccagccatctgttgtttgcccctcccccgtgccttccttgaccctggaaggtgccactcccact
gtcctttcctaataaaatgaggaaattgcatcgcattgtctgagtaggtgtcattctattctgggg
ggtggggtggggcaggacagcaagggggaggattgggaagacaatagcaggcatgctggggatgcg
gtgggctctatggcttctgaggcggaaagaaccagctggggctcgactagagcttgcggaaccctt
agagggcctatttcccatggtggtagaactagtattatgcccagtacatgaccttatgggactttc
ctacttggcagtacatctacgtattagtcatcgctattaccatggtgatgcggttttggcagtaca
tcaatgggcgtggatagcggtttgactcacggggatttccaagtctccaccccattgacgtcaatg
ggagtttgttttggcaccaaaatcaacgggactttccaaaatgtcgtaacaactccgccccattga
cgcaaatgggcggtaggcgtgtacggtgggaggtttatataagcagagctcgtttagtgaaccgtc
agatcgcctggagacgccatccacgctgttttgacctccatagaagagaattcgccgccaccatga
cagaatataaacctactgtcagactggcaactcgagacgacgtccctagggccgtgagaacattgg
ctgccgctttcgcggattatcccgctacacgccacacagttgatcctgatagacatattgaacggg
ttacagaattgcaagaactttttttgaccagggtaggattggacatcggtaaagtttgggtcgccg
acgacggggctgcagtggcagtgtggacgactccggagagcgttgaggccggggctgtatttgcag
aaattggtccccgaatggctgagcttagtggctctcgtctcgcggctcagcaacaaatggaaggac
tcctcgcccctcaccgccctaaagaaccagcttggttcctcgctactgtgggcgttagccccgatc
atcagggaaagggccttggttccgcggtggtattgcccggagtagaagccgcagaacgagccggag
tgccagcctttcttgaaacgtcagcgccaaggaatttgcccttctatgaacggctcggatttacag
ttactgctgacgttgaagtacccgagggcccacggacgtggtgcatgacgcgaaaacccggcgctt
gagtttaaaccgctgatcagcctcgactgtgccttctagttgccagccatctgttgtttgcccctc
ccccgtgccttccttgaccctggaaggtgccactcccactgtcctttcctaataaaatgaggaaat
tgcatcgcattgtctgagtaggtgtcattctattctggggggtggggtggggcaggacagcaaggg
ggaggattgggaagacaatagcaggcatgctggggatgcggtgggctctatggcttctgaggcgga
aagaaccagctggggctcgactagagcttgcggaacccttagtttaaacgggcccttaattaatcg
atgtaggatgttgcccctcctgacgcggtaggagaaggggagggtgccctgcatgtctgccgctgc
tcttgctcttgccgctgctgaggaggggggcgcatctgccgcagcaccggatgcatctgggaaaag
caaaaaaggggctcgtccctgtttccggaggaatttgcaagcggggtcttgcatgacggggaggca
aacccccgttcgccgcagtccggccggcccgagactcgaaccgggggtcctgcgactcaacccttg
gaaaataaccctccggctacagggagcgagccacttaatgctttcgctttccagcctaaccgctta
cgccgcgcgcggccagtggccaaaaaagctagcgcagcagccgccgcgcctggaaggaagccaaaa
ggagcgctcccccgttgtctgacgtcgcacacctgggttcgacacgcgggcggtaaccgcatggat
cacggcggacggccggatccggggttcgaaccccggtcgtccgccatgatacccttgcgaatttat
ccaccagaccacggaagagtgcccgcttacaggctctccttttgcacggtctagagcgtcaacgac
tgcgcacgcctcaccggccagagcgtcccgaccatggagcactttttgccgctgcgcaacatctgg
aaccgcgtccgcgactttccgcgcgcctccaccaccgccgccggcatcacctggatgtccaggtac
atctacggattacggggcccattggtatggctttttccccgtatccccccaggtgtctgcaggctc
aaagagcagcgagaagcgttcagaggaaagcgatcccgtgccaccttccccgtgcccgggctgtcc
ccgcacgctgccggctcggggatgcggggggagcgccggaccggagcggagccccgggcggctcgc
tgctgccccctagcgggggagggacgtaattacatccctgggggctttgggggggggctgtccctg
atatctataacaagaaaatatatatataataagttatcacgtaagtagaacatgaaataacaatat
aattatcgtatgagttaaatcttaaaagtcacgtaaaagataatcatgcgtcattttgactcacgc
ggtcgttatagttcaaaatcagtgacacttaccgcattgacaagcacgcctcacgggagctccaag
cggcgactgagatgtcctaaatgcacagcgacggattcgcgctatttagaaagagagagcaatatt
tcaagaatgcatgcgtcaattttacgcagactatctttctagggttaatctagctgcatcaggatc
atatcgtcgggtcttttttccggctcagtcatcgcccaagctggcgctatctgggcatcggggagg
aagaagcccgtgccttttcccgcgaggttgaagcggcatggaaagagtttgccgaggatgactgct
gctgcattgacgttgagcgaaaacgcacgtttaccatgatgattcgggaaggtgtggccatgcacg
cctttaacggtgaactgttcgttcaggccacctgggataccagttcgtcgcggcttttccggacac
agttccggatggtcagcccgaagcgcatcagcaacccgaacaataccggcgacagccggaactgcc
gtgccggtgtgcagattaatgacagcggtgcggcgctgggatattacgtcagcgaggacgggtatc
ctggctggatgccgcagaaatggacatggataccccgtgagttacccggcgggcgcgcttggcgta
atcatggtcatagctgtttcctgtgtgaaattgttatccgctcacaattccacacaacatacgagc
cggaagcataaagtgtaaagcctggggtgcctaatgagtgagctaactcacattaattgcgttgcg
ctcactgcccgctttccagtcgggaaacctgtcgtgccagctgcattaatgaatcggccaacgcgc
ggggagaggcggtttgcgtattgggcgctcttccgcttcctcgctcactgactcgctgcgctcggt
cgttcggctgcggcgagcggtatcagctcactcaaaggcggtaatacggttatccacagaatcagg
ggataacgcaggaaagaacatgtgagcaaaaggccagcaaaaggccaggaaccgtaaaaaggccgc
gttgctggcgtttttccataggctccgcccccctgacgagcatcacaaaaatcgacgctcaagtca
gaggtggcgaaacccgacaggactataaagataccaggcgtttccccctggaagctccctcgtgcg
ctctcctgttccgaccctgccgcttaccggatacctgtccgcctttctcccttcgggaagcgtggc
gctttctcatagctcacgctgtaggtatctcagttcggtgtaggtcgttcgctccaagctgggctg
tgtgcacgaaccccccgttcagcccgaccgctgcgccttatccggtaactatcgtcttgagtccaa
cccggtaagacacgacttatcgccactggcagcagccactggtaacaggattagcagagcgaggta
tgtaggcggtgctacagagttcttgaagtggtggcctaactacggctacactagaaggacagtatt
tggtatctgcgctctgctgaagccagttaccttcggaaaaagagttggtagctcttgatccggcaa
acaaaccaccgctggtagcggtggtttttttgtttgcaagcagcagattacgcgcagaaaaaaagg
atctcaagaagatcctttgatcttttctacggggtctgacgctcagtggaacgaaaactcacgtta
agggattttggtcatgagattatcaaaaaggatcttcacctagatccttttaaattaaaaatgaag
ttttaaatcaatctaaagtatatatgagtaaacttggtctgacagttaccaatgcttaatcagtga
ggcacctatctcagcgatctgtctatttcgttcatccatagttgcctgactccccgtcgtgtagat
aactacgatacgggagggcttaccatctggccccagtgctgcaatgataccgcgagacccacgctc
accggctccagatttatcagcaataaaccagccagccggaagggccgagcgcagaagtggtcctgc
aactttatccgcctccatccagtctattaattgttgccgggaagctagagtaagtagttcgccagt
taatagtttgcgcaacgttgttgccattgctacaggcatcgtggtgtcacgctcgtcgtttggtat
ggcttcattcagctccggttcccaacgatcaaggcgagttacatgatcccccatgttgtgcaaaaa
agcggttagctccttcggtcctccgatcgttgtcagaagtaagttggccgcagtgttatcactcat
ggttatggcagcactgcataattctcttactgtcatgccatccgtaagatgcttttctgtgactgg
tgagtactcaaccaagtcattctgagaatagtgtatgcggcgaccgagttgctcttgcccggcgtc
aatacgggataataccgcgccacatagcagaactttaaaagtgctcatcattggaaaacgttcttc
ggggcgaaaactctcaaggatcttaccgctgttgagatccagttcgatgtaacccactcgtgcacc
caactgatcttcagcatcttttactttcaccagcgtttctgggtgagcaaaaacaggaaggcaaaa
tgccgcaaaaaagggaataagggcgacacggaaatgttgaatactcat
SEQ ID NO: 32 (STXC0137)
ACTCTTCCTTTTTCAATATTATTGAAGCATTTATCAGGGTTATTGTCTCATGAGCGGATACATATT
TGAATGTATTTAGAAAAATAAACAAATAGGGGTTCCGCGCACATTTCCCCGAAAAGTGCCACCTAA
ATTGTAAGCGTTAATATTTTGTTAAAATTCGCGTTAAATTTTTGTTAAATCAGCTCATTTTTTAAC
CAATAGGCCGAAATCGGCAAAATCCCTTATAAATCAAAAGAATAGACCGAGATAGGGTTGAGTGTT
GTTCCAGTTTGGAACAAGAGTCCACTATTAAAGAACGTGGACTCCAACGTCAAAGGGCGAAAAACC
GTCTATCAGGGCGATGGCCCACTACGTGAACCATCACCCTAATCAAGTTTTTTGGGGTCGAGGTGC
CGTAAAGCACTAAATCGGAACCCTAAAGGGAGCCCCCGATTTAGAGCTTGACGGGGAAAGCCGGCG
AACGTGGCGAGAAAGGAAGGGAAGAAAGCGAAAGGAGCGGGCGCTAGGGCGCTGGCAAGTGTAGCG
GTCACGCTGCGCGTAACCACCACACCCGCCGCGCTTAATGCGCCGCTACAGGGCGCGTCCCATTCG
CCATTCAGGCTGCGCAACTGTTGGGAAGGGCGATCGGTGCGGGCCTCTTCGCTATTACGCCAGCTG
GCGAAAGGGGGATGTGCTGCAAGGCGATTAAGTTGGGTAACGCCAGGGTTTTCCCAGTCACGACGT
TGTAAAACGACGGCCAGTGAGCGCGCCTCGTTCATTCACGTTTTTGAACCCGTGGAGGACGGGCAG
ACTCGCGGTGCAAATGTGTTTTACAGCGTGATGGAGCAGATGAAGATGCTCGACACGCTGCAGAAC
ACGCAGCTAGATTAACCCTAGAAAGATAATCATATTGTGACGTACGTTAAAGATAATCATGTGTAA
AATTGACGCATGTGTTTTATCGGTCTGTATATCGAGGTTTATTTATTAATTTGAATAGATATTAAG
TTTTATTATATTTACACTTACATACTAATAATAAATTCAACAAACAATTTATTTATGTTTATTTAT
TTATTAAAAAAAACAAAAACTCAAAATTTCTTCTATAAAGTAACAAAACTTTTATGGAGGGGTGGA
GTCGTGACGTGAATTACGTCATAGGGTTAGGGAGGTCCTGTATTAGAGGTCACGTGAGTGTTTTGC
GACATTTTGCGACACCATGTGGTCACGCTGGGTATTTAAGCCCGAGTGAGCACGCAGGGTCTCCAT
TTTGAAGCGGGAGGTTTGAACGCGCAGCCGCCATGCCGGGGTTTTACGAGATTGTGATTAAGGTCC
CCAGCGACCTTGACGAGCATCTGCCCGGCATTTCTGACAGCTTTGTGAACTGGGTGGCCGAGAAGG
AATGGGAGTTGCCGCCAGATTCTGACATGGATCTGAATCTGATTGAGCAGGCACCCCTGACCGTGG
CCGAGAAGCTGCAGCGCGACTTTCTGACGGAATGGCGCCGTGTGAGTAAGGCCCCGGAGGCCCTTT
TCTTTGTGCAATTTGAGAAGGGAGAGAGCTACTTCCACATGCACGTGCTCGTGGAAACCACCGGGG
TGAAATCCATGGTTTTGGGACGTTTCCTGAGTCAGATTCGCGAAAAACTGATTCAGAGAATTTACC
GCGGGATCGAGCCGACTTTGCCAAACTGGTTCGCGGTCACAAAGACCAGAAATGGCGCCGGAGGCG
GGAACAAGGTGGTGGATGAGTGCTACATCCCCAATTACTTGCTCCCCAAAACCCAGCCTGAGCTCC
AGTGGGCGTGGACTAATATGGAACAGTATTTAAGCGCCTGTTTGAATCTCACGGAGCGTAAACGGT
TGGTGGCGCAGCATCTGACGCACGTGTCGCAGACGCAGGAGCAGAACAAAGAGAATCAGAATCCCA
ATTCTGATGCGCCGGTGATCAGATCAAAAACTTCAGCCAGGTACATGGAGCTGGTCGGGTGGCTCG
TGGACAAGGTGAGTTTGGGGACCCTTGATTGTTCTTTCTTTTTCGCTATTGTAAAATTCATGTTAT
ATGGAGGGGGCAAAGTTTTCAGGGTGTTGTTTAGAATGGGAAGATGTCCCTTGTATCACCATGGAC
CCTCATGATAATTTTGTTTCTTTCACTTTCTACTCTGTTGACAACCGTTGTCTCCTCTTATTTTCT
TTTCATTTTCTGTAACTTTTTCGTTAAACTTTAGCTTGCATTTGTAACGAATTTTTAAATTCACTT
TTGTTTATTTGTCAGATTGTAAGTACTTTCTCTAATCACTTTTTTTTCAAGGCAATCAGGGTATAT
TATATTGTACTTCAGCACAGTTTTAGAGAACATAACTTCGTATAAAGTATACTATACGAAGTTATC
GGGCCCCTCTGCTAACCATGTTCATGCCTTCTTCTTTTTCCTACAGATGTCAGAACTCATTAAAGA
GAATATGCACATGAAGCTGTATATGGAAGGTACTGTAGACAACCACCATTTCAAATGCACGTCCGA
AGGTGAGGGGAAGCCATACGAGGGTACCCAAACTATGCGCATCAAAGTGGTTGAGGGTGGCCCCCT
GCCATTCGCATTCGACATCCTGGCAACTAGCTTTCTTTACGGTTCCAAGACATTCATAAATCATAC
CCAGGGTATTCCCGATTTCTTCAAACAATCCTTCCCGGAAGGGTTTACTTGGGAGCGGGTCACGAC
ATATGAAGACGGGGGTGTTCTTACAGCCACACAGGATACGAGTTTGCAAGACGGTTGTCTTATCTA
TAACGTGAAGATTCGGGGTGTGAATTTCACATCCAATGGCCCGGTGATGCAGAAAAAAACACTGGG
CTGGGAAGCATTTACGGAGACGTTGTATCCCGCCGATGGAGGTCTCGAGGGCCGAAACGATATGGC
CCTCAAGTTGGTAGGTGGTTCTCACCTTATAGCAAACATTAAGACCACGTATCGATCAAAAAAACC
CGCTAAGAATCTGAAAATGCCAGGCGTGTATTATGTTGATTACAGACTGGAGCGAATAAAAGAGGC
TAACAATGAGACCTACGTCGAACAGCATGAAGTCGCTGTAGCTAGATATTGCGACCTCCCGTCAAA
GTTGGGCCATAAATTGAATTAACCTCAGGTGCAGGCTGCCTATCAGAAGGTGGTGGCTGGTGTGGC
CAATGCCCTGGCTCACAAATACCACTGAGATCTTTTTCCCTCTGCCAAAAATTATGGGGACATCAT
GAAGCCCCTTGAGCATCTGACTTCTGGCTAATAAAGGAAATTTATTTTCATTGCAATAGTGTGTTG
GAATTTTTTGTGTCTCTCACTCGGAAGGACATATGGGAGGGCAAATCATTTAAAACATCAGAATGA
GTATTTGGTTTAGAGTTTGGCAACATATGCCCATATGCTGGCTGCCATGAACAAAGGTTGGCTATA
AAGAGGTCATCAGTATATGAAACAGCCCCCTGCTGTCCATTCCTTATTCCATAGAAAAGCCTTGAC
TTGAGGTTAGATTTTTTTTATATTTTGTTTTGTGTTATTTTTTTCTTTAACATCCCTAAAATTTTC
CTTACATGTTTTACTAGCCAGATTTTTCCTCCTCTCCTGACTACTCCCAGTCATAGCTGTCCCTCT
TCTCTTATGGAGATCATAACTTCGTATAAAGTATACTATACGAAGTTATAATTGTTATAATTAAAT
GATAAGGTAGAATATTTCTGCATATAAATTCTGGCTGGCGTGGAAATATTCTTATTGGTAGAAACA
ACTACACCCTGGTCATCATCCTGCCTTTCTCTTTATGGTTACAATGATATACACTGTTTGAGATGA
GGATAAAATACTCTGAGTCCAAACCGGGCCCCTCTGCTAACCATGTTCATGCCTTCTTCTTTTTCC
TACAGGGGATTACCTCGGAGAAGCAGTGGATCCAGGAGGACCAGGCCTCATACATCTCCTTCAATG
CGGCCTCCAACTCGCGGTCCCAAATCAAGGCTGCCTTGGACAATGCGGGAAAGATTATGAGCCTGA
CTAAAACCGCCCCCGACTACCTGGTGGGCCAGCAGCCCGTGGAGGACATTTCCAGCAATCGGATTT
ATAAAATTTTGGAACTAAACGGGTACGATCCCCAATATGCGGCTTCCGTCTTTCTGGGATGGGCCA
CGAAAAAGTTCGGCAAGAGGAACACCATCTGGCTGTTTGGGCCTGCAACTACCGGGAAGACCAACA
TCGCGGAGGCCATAGCCCACACTGTGCCCTTCTACGGGTGCGTAAACTGGACCAATGAGAACTTTC
CCTTCAACGACTGTGTCGACAAGATGGTGATCTGGTGGGAGGAGGGGAAGATGACCGCCAAGGTCG
TGGAGTCGGCCAAAGCCATTCTCGGAGGAAGCAAGGTGCGCGTGGACCAGAAATGCAAGTCCTCGG
CCCAGATAGACCCGACTCCCGTGATCGTCACCTCCAACACCAACATGTGCGCCGTGATTGACGGGA
ACTCAACGACCTTCGAACACCAGCAGCCGTTGCAAGACCGGATGTTCAAATTTGAACTCACCCGCC
GTCTGGATCATGACTTTGGGAAGGTCACCAAGCAGGAAGTCAAAGACTTTTTCCGGTGGGCAAAGG
ATCACGTGGTTGAGGTGGAGCATGAATTCTACGTCAAAAAGGGTGGAGCCAAGAAAAGACCCGCCC
CCAGTGACGCAGATATAAGTGAGCCCAAACGGGTGCGCGAGTCAGTTGCGCAGCCATCGACGTCAG
ACGCGGAAGCTTCGATCAACTACGCAGACAGGTACCAAAACAAATGTTCTCGTCACGTGGGCATGA
ATCTGATGCTGTTTCCCTGCAGACAATGCGAGAGAATGAATCAGAATTCAAATATCTGCTTCACTC
ACGGACAGAAAGACTGTTTAGAGTGCTTTCCCGTGTCAGAATCTCAACCCGTTTCTGTCGTCAAAA
AGGCGTATCAGAAACTGTGCTACATTCATCATATCATGGGAAAGGTGCCAGACGCTTGCACTGCCT
GCGATCTGGTCAATGTGGATTTGGATGACTGCATCTTTGAACAATAAATGATTTGTAAATAAATTT
AGTAGTCATGTCTTTTGTTGATCACCCTCCAGATTGGTTGGAAGAAGTTGGTGAAGGTCTTCGCGA
GTTTTTGGGCCTTGAAGCGGGCCCACCGAAACCAAAACCCAATCAGCAGCATCAAGATCAAGCCCG
TGGTCTTGTGCTGCCTGGTTATAACTATCTCGGACCCGGAAACGGTCTCGATCGAGGAGAGCCTGT
CAACAGGGCAGACGAGGTCGCGCGAGAGCACGACATCTCGTACAACGAGCAGCTTGAGGCGGGAGA
CAACCCCTACCTCAAGTACAACCACGCGGACGCCGAGTTTCAGGAGAAGCTCGCCGACGACACATC
CTTCGGGGGAAACCTCGGAAAGGCAGTCTTTCAGGCCAAGAAAAGGGTTCTCGAACCTTTTGGCCT
GGTTGAAGAGGGTGCTAAGACGGCCCCTACCGGAAAGCGGATAGACGACCACTTTCCAAAAAGAAA
GAAGGCTCGGACCGAAGAGGACTCCAAGCCTTCCACCTCGTCAGACGCCGAAGCTGGACCCAGCGG
ATCCCAGCAGCTGCAAATCCCAGCCCAACCAGCCTCAAGTTTGGGAGCTGATACAATGTCTGCGGG
AGGTGGCGGCCCATTGGGCGACAATAACCAAGGTGCCGATGGAGTGGGCAATGCCTCGGGAGATTG
GCATTGCGATTCCACGTGGATGGGGGACAGAGTCGTCACCAAGTCCACCCGAACCTGGGTGCTGCC
CAGCTACAACAACCACCAGTACCGAGAGATCAAAAGCGGCTCCGTCGACGGAAGCAACGCCAACGC
CTACTTTGGATACAGCACCCCCTGGGGGTACTTTGACTTTAACCGCTTCCACAGCCACTGGAGCCC
CCGAGACTGGCAAAGACTCATCAACAACTACTGGGGCTTCAGACCCCGGTCCCTCAGAGTCAAAAT
CTTCAACATTCAAGTCAAAGAGGTCACGGTGCAGGACTCCACCACCACCATCGCCAACAACCTCAC
CTCCACCGTCCAAGTGTTTACGGACGACGACTACCAGCTGCCCTACGTCGTCGGCAACGGGACCGA
GGGATGCCTGCCGGCCTTCCCTCCGCAGGTCTTTACGCTGCCGCAGTACGGTTACGCGACGCTGAA
CCGCGACAACACAGAAAATCCCACCGAGAGGAGCAGCTTCTTCTGCCTAGAGTACTTTCCCAGCAA
GATGCTGAGAACGGGCAACAACTTTGAGTTTACCTACAACTTTGAGGAGGTGCCCTTCCACTCCAG
CTTCGCTCCCAGTCAGAACCTGTTCAAGCTGGCCAACCCGCTGGTGGACCAGTACTTGTACCGCTT
CGTGAGCACAAATAACACTGGCGGAGTCCAGTTCAACAAGAACCTGGCCGGGAGATACGCCAACAC
CTACAAAAACTGGTTCCCGGGGCCCATGGGCCGAACCCAGGGCTGGAACCTGGGCTCCGGGGTCAA
CCGCGCCAGTGTCAGCGCCTTCGCCACGACCAATAGGATGGAGCTCGAGGGCGCGAGTTACCAGGT
GCCCCCGCAGCCGAACGGCATGACCAACAACCTCCAGGGCAGCAACACCTATGCCCTGGAGAACAC
TATGATCTTCAACAGCCAGCCGGCGAACCCGGGCACCACCGCCACGTACCTCGAGGGCAACATGCT
CATCACCAGCGAGAGCGAGACGCAGCCGGTGAACCGCGTGGCGTACAACGTCGGCGGGCAGATGGC
CACCAACAACCAGAGCTCCACCACTGCCCCCGCGACCGGCACGTACAACCTCCAGGAAATCGTGCC
CGGCAGCGTGTGGATGGAGAGGGACGTGTACCTCCAAGGACCCATCTGGGCCAAGATCCCAGAGAC
GGGGGCGCACTTTCACCCCTCTCCGGCCATGGGCGGATTCGGACTCAAACACCCACCGCCCATGAT
GCTCATCAAGAACACGCCTGTGCCCGGAAATATCACCAGCTTCTCGGACGTGCCCGTCAGCAGCTT
CATCACCCAGTACAGCACCGGGCAGGTCACCGTGGAGATGGAGTGGGAGCTCAAGAAGGAAAACTC
CAAGAGGTGGAACCCAGAGATCCAGTACACAAACAACTACAACGACCCCCAGTTTGTGGACTTTGC
CCCGGACAGCACCGGGGAATACAGAACCACCAGACCTATCGGAACCCGATACCTTACCCGACCCCT
TTAATTGCTTGTTAATCAATAAACCGTTTAATTCGTTTCAGTTGAACTTTGGTCTCTGCGTATTTC
TTTCTTATCTAGTTTCCATGGCTACGTAGATAAGTAGCATGGCGGGTTAATCATTAACTACAGCCC
GGGCGTTTAAACAGCGGGCGGAGGGGTGGAGTCGTGACGTGAATTACGTCATAGGGTTAGGGAGGT
CCTGTATTAGAGGTCACGTGAGTGTTTTGCGACATTTTGCGACACCATGTGGTCCGCGGCCGCAAG
GATCTGCGATCGCTCCGGTGCCCGTCAGTGGGCAGAGCGCACATCGCCCACAGTCCCCGAGAAGTT
GGGGGGAGGGGTCGGCAATTGAACGGGTGCCTAGAGAAGGTGGCGCGGGGTAAACTGGGAAAGTGA
TGTCGTGTACTGGCTCCGCCTTTTTCCCGAGGGTGGGGGAGAACCGTATATAAGTGCAGTAGTCGC
CGTGAACGTTCTTTTTCGCAACGGGTTTGCCGCCAGAACACAGCTGAAGCTTCGAGGGGCTCGCAT
CTCTCCTTCACGCGCCCGCCGCCCTACCTGAGGCCGCCATCCACGCCGGTTGAGTCGCGTTCTGCC
GCCTCCCGCCTGTGGTGCCTCCTGAACTGCGTCCGCCGTCTAGGTAAGTTTAAAGCTCAGGTCGAG
ACCGGGCCTTTGTCCGGCGCTCCCTTGGAGCCTACCTAGACTCAGCCGGCTCTCCACGCTTTGCCT
GACCCTGCTTGCTCAACTCTACGTCTTTGTTTCGTTTTCTGTTCTGCGCCGTTACAGATCCAAGCT
GTGACCGGCGCCTACGATATCGCCACCATGATTAAGATCGCTACGCGGAAGTACCTGGGGAAACAG
AACGTCTACGACATAGGTGTGGAGCGCGATCACAACTTTGCTCTGAAAAATGGATTTATCGCCAGC
AACTGTAGGGAGTTGATTTCAGACTATGCACCAGATTGTTTTGTGTTAATAGAAATGAATGGCAAG
TTAGTCAAAACTACGATTGAAGAACTCATTCCACTCAAATATACCCGAAATTAAGTGCATGACCCG
CAAGCCCGGTGCCTGAAATCAACCTCTGGATTACAAAATTTGTGAAAGATTGACTGGTATTCTTAA
CTATGTTGCTCCTTTTACGCTATGTGGATACGCTGCTTTAATGCCTTTGTATCATGCGTTAACTAA
ACTTGTTTATTGCAGCTTATAATGGTTACAAATAAAGCAATAGCATCACAAATTTCACAAATAAAG
CATTTTTTTCACTGCATTCTAGTTGTGGTTTGTCCAAACTCATCAATGTATCTTATCATGTCTGGA
ATTGACTCAAATGATGTCAATTAGTCTATCAGAAGCTCATCTGGTCTCCCTTCCGGGGGACAAGAC
ATCCCTGTTTAATATTTAAACAGCAGTGTTCCCAAACTGGGTTCTTATATCCCTTGCTCTGGTCAA
CCAGGTTGCAGGGTTTCCTGTCCTCACAGGAACGAAGTCCCTAAAGAAACAGTGGCAGCCAGGTTT
AGCCCCGGAATTGACTGGATTCCTTTTTTAGGGCCCATTGGTATGGCGATATCTATAACAAGAAAA
TATATATATAATAAGTTATCACGTAAGTAGAACATGAAATAACAATATAATTATCGTATGAGTTAA
ATCTTAAAAGTCACGTAAAAGATAATCATGCGTCATTTTGACTCACGCGGTCGTTATAGTTCAAAA
TCAGTGACACTTACCGCATTGACAAGCACGCCTCACGGGAGCTCCAAGCGGCGACTGAGATGTCCT
AAATGCACAGCGACGGATTCGCGCTATTTAGAAAGAGAGAGCAATATTTCAAGAATGCATGCGTCA
ATTTTACGCAGACTATCTTTCTAGGGTTAATCTAGCTGCATCAGGATCATATCGTCGGGTCTTTTT
TCCGGCTCAGTCATCGCCCAAGCTGGCGCTATCTGGGCATCGGGGAGGAAGAAGCCCGTGCCTTTT
CCCGCGAGGTTGAAGCGGCATGGAAAGAGTTTGCCGAGGATGACTGCTGCTGCATTGACGTTGAGC
GAAAACGCACGTTTACCATGATGATTCGGGAAGGTGTGGCCATGCACGCCTTTAACGGTGAACTGT
TCGTTCAGGCCACCTGGGATACCAGTTCGTCGCGGCTTTTCCGGACACAGTTCCGGATGGTCAGCC
CGAAGCGCATCAGCAACCCGAACAATACCGGCGACAGCCGGAACTGCCGTGCCGGTGTGCAGATTA
ATGACAGCGGTGCGGCGCTGGGATATTACGTCAGCGAGGACGGGTATCCTGGCTGGATGCCGCAGA
AATGGACATGGATACCCCGTGAGTTACCCGGCGGGCGCGCTTGGCGTAATCATGGTCATAGCTGTT
TCCTGTGTGAAATTGTTATCCGCTCACAATTCCACACAACATACGAGCCGGAAGCATAAAGTGTAA
AGCCTGGGGTGCCTAATGAGTGAGCTAACTCACATTAATTGCGTTGCGCTCACTGCCCGCTTTCCA
GTCGGGAAACCTGTCGTGCCAGCTGCATTAATGAATCGGCCAACGCGCGGGGAGAGGCGGTTTGCG
TATTGGGCGCTCTTCCGCTTCCTCGCTCACTGACTCGCTGCGCTCGGTCGTTCGGCTGCGGCGAGC
GGTATCAGCTCACTCAAAGGCGGTAATACGGTTATCCACAGAATCAGGGGATAACGCAGGAAAGAA
CATGTGAGCAAAAGGCCAGCAAAAGGCCAGGAACCGTAAAAAGGCCGCGTTGCTGGCGTTTTTCCA
TAGGCTCCGCCCCCCTGACGAGCATCACAAAAATCGACGCTCAAGTCAGAGGTGGCGAAACCCGAC
AGGACTATAAAGATACCAGGCGTTTCCCCCTGGAAGCTCCCTCGTGCGCTCTCCTGTTCCGACCCT
GCCGCTTACCGGATACCTGTCCGCCTTTCTCCCTTCGGGAAGCGTGGCGCTTTCTCATAGCTCACG
CTGTAGGTATCTCAGTTCGGTGTAGGTCGTTCGCTCCAAGCTGGGCTGTGTGCACGAACCCCCCGT
TCAGCCCGACCGCTGCGCCTTATCCGGTAACTATCGTCTTGAGTCCAACCCGGTAAGACACGACTT
ATCGCCACTGGCAGCAGCCACTGGTAACAGGATTAGCAGAGCGAGGTATGTAGGCGGTGCTACAGA
GTTCTTGAAGTGGTGGCCTAACTACGGCTACACTAGAAGGACAGTATTTGGTATCTGCGCTCTGCT
GAAGCCAGTTACCTTCGGAAAAAGAGTTGGTAGCTCTTGATCCGGCAAACAAACCACCGCTGGTAG
CGGTGGTTTTTTTGTTTGCAAGCAGCAGATTACGCGCAGAAAAAAAGGATCTCAAGAAGATCCTTT
GATCTTTTCTACGGGGTCTGACGCTCAGTGGAACGAAAACTCACGTTAAGGGATTTTGGTCATGAG
ATTATCAAAAAGGATCTTCACCTAGATCCTTTTAAATTAAAAATGAAGTTTTAAATCAATCTAAAG
TATATATGAGTAAACTTGGTCTGACAGTTACCAATGCTTAATCAGTGAGGCACCTATCTCAGCGAT
CTGTCTATTTCGTTCATCCATAGTTGCCTGACTCCCCGTCGTGTAGATAACTACGATACGGGAGGG
CTTACCATCTGGCCCCAGTGCTGCAATGATACCGCGAGACCCACGCTCACCGGCTCCAGATTTATC
AGCAATAAACCAGCCAGCCGGAAGGGCCGAGCGCAGAAGTGGTCCTGCAACTTTATCCGCCTCCAT
CCAGTCTATTAATTGTTGCCGGGAAGCTAGAGTAAGTAGTTCGCCAGTTAATAGTTTGCGCAACGT
TGTTGCCATTGCTACAGGCATCGTGGTGTCACGCTCGTCGTTTGGTATGGCTTCATTCAGCTCCGG
TTCCCAACGATCAAGGCGAGTTACATGATCCCCCATGTTGTGCAAAAAAGCGGTTAGCTCCTTCGG
TCCTCCGATCGTTGTCAGAAGTAAGTTGGCCGCAGTGTTATCACTCATGGTTATGGCAGCACTGCA
TAATTCTCTTACTGTCATGCCATCCGTAAGATGCTTTTCTGTGACTGGTGAGTACTCAACCAAGTC
ATTCTGAGAATAGTGTATGCGGCGACCGAGTTGCTCTTGCCCGGCGTCAATACGGGATAATACCGC
GCCACATAGCAGAACTTTAAAAGTGCTCATCATTGGAAAACGTTCTTCGGGGCGAAAACTCTCAAG
GATCTTACCGCTGTTGAGATCCAGTTCGATGTAACCCACTCGTGCACCCAACTGATCTTCAGCATC
TTTTACTTTCACCAGCGTTTCTGGGTGAGCAAAAACAGGAAGGCAAAATGCCGCAAAAAAGGGAAT
AAGGGCGACACGGAAATGTTGAATACTCAT
SEQ ID NO: 33 (STXC0136)
ACTCTTCCTTTTTCAATATTATTGAAGCATTTATCAGGGTTATTGTCTCATGAGCGGATACATATT
TGAATGTATTTAGAAAAATAAACAAATAGGGGTTCCGCGCACATTTCCCCGAAAAGTGCCACCTAA
ATTGTAAGCGTTAATATTTTGTTAAAATTCGCGTTAAATTTTTGTTAAATCAGCTCATTTTTTAAC
CAATAGGCCGAAATCGGCAAAATCCCTTATAAATCAAAAGAATAGACCGAGATAGGGTTGAGTGTT
GTTCCAGTTTGGAACAAGAGTCCACTATTAAAGAACGTGGACTCCAACGTCAAAGGGCGAAAAACC
GTCTATCAGGGCGATGGCCCACTACGTGAACCATCACCCTAATCAAGTTTTTTGGGGTCGAGGTGC
CGTAAAGCACTAAATCGGAACCCTAAAGGGAGCCCCCGATTTAGAGCTTGACGGGGAAAGCCGGCG
AACGTGGCGAGAAAGGAAGGGAAGAAAGCGAAAGGAGCGGGCGCTAGGGCGCTGGCAAGTGTAGCG
GTCACGCTGCGCGTAACCACCACACCCGCCGCGCTTAATGCGCCGCTACAGGGCGCGTCCCATTCG
CCATTCAGGCTGCGCAACTGTTGGGAAGGGCGATCGGTGCGGGCCTCTTCGCTATTACGCCAGCTG
GCGAAAGGGGGATGTGCTGCAAGGCGATTAAGTTGGGTAACGCCAGGGTTTTCCCAGTCACGACGT
TGTAAAACGACGGCCAGTGAGCGCGCCTCGTTCATTCACGTTTTTGAACCCGTGGAGGACGGGCAG
ACTCGCGGTGCAAATGTGTTTTACAGCGTGATGGAGCAGATGAAGATGCTCGACACGCTGCAGAAC
ACGCAGCTAGATTAACCCTAGAAAGATAATCATATTGTGACGTACGTTAAAGATAATCATGTGTAA
AATTGACGCATGTGTTTTATCGGTCTGTATATCGAGGTTTATTTATTAATTTGAATAGATATTAAG
TTTTATTATATTTACACTTACATACTAATAATAAATTCAACAAACAATTTATTTATGTTTATTTAT
TTATTAAAAAAAACAAAAACTCAAAATTTCTTCTATAAAGTAACAAAACTTTTATTTCTTTCCTGC
GTTATCCCCTGATTCTGTGGATAACCGTATTACCGCCTTTGAGTGAGCTGATACCGCTCGCCGCAG
CCGAACGACCGAGCGCAGCGAGTCAGTGAGCGAGGAAGCGGAAGAGCGCCCAATACGCAAACCGCC
TCTCCCCGCGCGTTGGCCGATTCATTAATGCAGCTGGCACGACAGGTTTCCCGACTGGAAAGCGGG
CAGTGAGCGCAACGCAATTAATGTGAGTTAGCTCACTCATTAGGCACCCCAGGCTTTACACTTTAT
GCTTCCGGCTCGTATGTTGTGTGGAATTGTGAGCGGATAACAATTTCACACAGGAAACAGCTATGA
CCATGATTACGCCAAGCTTTTGGCCACTCCCTCTCTGCGCGCTCGCTCGCTCACTGAGGCCGGGCG
ACCAAAGGTCGCCCGACGCCCGGGCTTTGCCCGGGCGGCCTCAGTGAGCGAGCGAGCGCGCAGAGA
GGGAGTGGCCAACTCCATCACTAGGGGTTCCTCTGCAGCCGCGACCGGCCAAGGTTTAATGATAGG
CTGCAACGGGATGTTGGGAATATGTTGCACTGGTCCGTGAGGGTACCAACTTGTTTATTGCAGCTT
ATAATGGTTACAAATAAAGCAATAGCATCACAAATTTCACAAATAAAGCATTTTTTTCACTGCATT
CTAGTTGTGGTTTGTCCAAACTCATCAATGTATCTTATCATGTCTGACCGGTTCACTTGAGCTCGA
GATCTGAGTACTTGTACAGCTCGTCCATGCCGAGAGTGATCCCGGCGGCGGTCACGAACTCCAGCA
GGACCATGTGATCGCGCTTCTCGTTGGGGTCTTTGCTCAGGGCGGACTGGGTGCTCAGGTAGTGGT
TGTCGGGCAGCAGCACGGGGCCGTCGCCGATGGGGGTGTTCTGCTGGTAGTGGTCGGCGAGCTGCA
CGCTGCCGTCCTCGATGTTGTGGCGGATCTTGAAGTTCACCTTGATGCCGTTCTTCTGCTTGTCGG
CCATGATATAGACGTTGTGGCTGTTGTAGTTGTACTCCAGCTTGTGCCCCAGGATGTTGCCGTCCT
CCTTGAAGTCGATGCCCTTCAGCTCGATGCGGTTCACCAGGGTGTCGCCCTCGAACTTCACCTCGG
CGCGGGTCTTGTAGTTGCCGTCGTCCTTGAAGAAGATGGTGCGCTCCTGGACGTAGCCTTCGGGCA
TGGCGGACTTGAAGAAGTCGTGCTGCTTCATGTGGTCGGGGTAGCGGCTGAAGCACTGCACGCCGT
AGGTCAGGGTGGTCACGAGGGTGGGCCAGGGCACGGGCAGCTTGCCGGTGGTGCAGATGAACTTCA
GGGTCAGCTTGCCGTAGGTGGCATCGCCCTCGCCCTCGCCGGACACGCTGAACTTGTGGCCGTTTA
CGTCGCCGTCCAGCTCGACCAGGATGGGCACCACCCCGGTGAACAGCTCCTCGCCCTTGCTCACCA
TGGTGGCGGCTTAAGGGTTCGATCCTCTAGAGTCCGGAGGCTGGATCGGTCCCGGTGTCTACTATG
GAGGTCAAAACAGCGTGGATGGCGTCTCCAGGCGATCTGACGGTTCACTAAACGAGCTCTGCTTAT
ATAGACCTCCCACCGTACACGCCTACCGCCCATTTGCGTCAATGGGGCGGAGTTGTTACGACATTT
TGGAAAGTCCCGTTGATTTTGGTGCCAAAACAAACTCCCATTGACGTCAATGGGGTGGAGACTTGG
AAATCCCCGTGAGTCAAACCGCTATCCACGCCCATTGATGTACTGCCAAAACCGCATCACCATGGT
AATAGCGATGACTAATACGTAGATGTACTGCCAAGTAGGAAAGTCCCATAAGGTCATGTACTGGGC
ATAATGCCAGGCGGGCCATTTACCGTCATTGACGTCAATAGGGGGCGTACTTGGCATATGATACAC
TTGATGTACTGCCAAGTGGGCAGTTTACCGTAAATACTCCACCCATTGACGTCAATGGAAAGTCCC
TATTGGCGTTACTATGGGAACATACGTCATTATTGACGTCAATGGGGGGGGGTCGTTGGGCGGTCA
GCCAGGCGGGCCATTTACCGTAAGTTATGTAACGCGGAACTCCATATATGGGCTATGAACTAATGA
CCCCGTAATTGATTACTATTAATAACTAGTCAATAATCAATGTCAACGCGTATGGTACCTGCGGAG
GATGCCGAGGATAACCTTGTTACTAGCCTCCGCCTGGCCGTTGGACTGTGGATAATATGGCGTAGA
GGATCCTCTGCGCGCTCGCTCGCTCACTGAGGCCGCCCGGGCAAAGCCCGGGCGTCGGGCGACCTT
TGGTCGCCCGGCCTCAGTGAGCGAGCGAGCGCGCAGAGAATTCACTGGCCGTCGTTTTACAACGTC
GTGACTGGGAAAACCCTGGCGTTACCCAACTTAATCGCCTTGCAGCACATCCCCCTTTCGCCAGCT
GGCGTAATAGCGAAGAGGCCCGCACCGATCGCCCTTCCCAACAGTTGCGCAGCCTGAATGGCGAAT
GGTCTAGAGCTAGCGAATTCGAATTTAAATCGGATCCGCGGCCGCAAGGATCTGCGATCGCTCCGG
TGCCCGTCAGTGGGCAGAGCGCACATCGCCCACAGTCCCCGAGAAGTTGGGGGGAGGGGTCGGCAA
TTGAACGGGTGCCTAGAGAAGGTGGCGCGGGGTAAACTGGGAAAGTGATGTCGTGTACTGGCTCCG
CCTTTTTCCCGAGGGTGGGGGAGAACCGTATATAAGTGCAGTAGTCGCCGTGAACGTTCTTTTTCG
CAACGGGTTTGCCGCCAGAACACAGCTGAAGCTTCGAGGGGCTCGCATCTCTCCTTCACGCGCCCG
CCGCCCTACCTGAGGCCGCCATCCACGCCGGTTGAGTCGCGTTCTGCCGCCTCCCGCCTGTGGTGC
CTCCTGAACTGCGTCCGCCGTCTAGGTAAGTTTAAAGCTCAGGTCGAGACCGGGCCTTTGTCCGGC
GCTCCCTTGGAGCCTACCTAGACTCAGCCGGCTCTCCACGCTTTGCCTGACCCTGCTTGCTCAACT
CTACGTCTTTGTTTCGTTTTCTGTTCTGCGCCGTTACAGATCCAAGCTGTGACCGGCGCCTACGAT
ATCGCCACCATGAAAACATTTAACATTTCTCAACAGGATCTAGAATTAGTAGAAGTAGCGACAGAG
AAGATTACAATGCTTTATGAGGATAATAAACATCATGTGGGAGCGGCAATTCGTACGAAAACAGGA
GAAATCATTTCGGCAGTACATATTGAAGCGTATATAGGACGAGTAACTGTTTGTGCAGAAGCCATT
GCGATTGGTAGTGCAGTTTCGAATGGACAAAAGGATTTTGACACGATTGTAGCTGTTAGACACCCT
TATTCTGACGAAGTAGATAGAAGTATTCGAGTGGTAAGTCCTTGTGGTATGTGCCTTTCATACGAG
ACCGAGATCCTGACTGTCGAGTACGGATTGCTTCCTATCGGCAAAATCGTGGAGAAGAGGATTGAA
TGTACCGTCTATTCAGTCGATAATAATGGGAACATCTACACACAGCCCGTGGCTCAATGGCACGAC
AGAGGAGAGCAGGAAGTTTTTGAATACTGTCTCGAGGACGGATCCCTCATCCGCGCTACTAAAGAT
CATAAGTTTATGACCGTGGACGGCCAGATGCTGCCAATTGACGAAATTTTTGAACGAGAGCTGGAT
CTGATGAGAGTCGACAACCTTCCAAACTGAGTGCATGACCCGCAAGCCCGGTGCCTGAAATCAACC
TCTGGATTACAAAATTTGTGAAAGATTGACTGGTATTCTTAACTATGTTGCTCCTTTTACGCTATG
TGGATACGCTGCTTTAATGCCTTTGTATCATGCGTTAACTAAACTTGTTTATTGCAGCTTATAATG
GTTACAAATAAAGCAATAGCATCACAAATTTCACAAATAAAGCATTTTTTTCACTGCATTCTAGTT
GTGGTTTGTCCAAACTCATCAATGTATCTTATCATGTCTGGAATTGACTCAAATGATGTCAATTAG
TCTATCAGAAGCTATCTGGTCTCCCTTCCGGGGGACAAGACATCCCTGTTTAATATTTAAACAGCA
GTGTTCCCAAACTGGGTTCTTATATCCCTTGCTCTGGTCAACCAGGTTGCAGGGTTTCCTGTCCTC
ACAGGAACGAAGTCCCTAAAGAAACAGTGGCAGCCAGGTTTAGCCCCGGAATTGACTGGATTCCTT
TTTTAGGGCCCATTGGTATGGCTGATATCTATAACAAGAAAATATATATATAATAAGTTATCACGT
AAGTAGAACATGAAATAACAATATAATTATCGTATGAGTTAAATCTTAAAAGTCACGTAAAAGATA
ATCATGCGTCATTTTGACTCACGCGGTCGTTATAGTTCAAAATCAGTGACACTTACCGCATTGACA
AGCACGCCTCACGGGAGCTCCAAGCGGCGACTGAGATGTCCTAAATGCACAGCGACGGATTCGCGC
TATTTAGAAAGAGAGAGCAATATTTCAAGAATGCATGCGTCAATTTTACGCAGACTATCTTTCTAG
GGTTAATCTAGCTGCATCAGGATCATATCGTCGGGTCTTTTTTCCGGCTCAGTCATCGCCCAAGCT
GGCGCTATCTGGGCATCGGGGAGGAAGAAGCCCGTGCCTTTTCCCGCGAGGTTGAAGCGGCATGGA
AAGAGTTTGCCGAGGATGACTGCTGCTGCATTGACGTTGAGCGAAAACGCACGTTTACCATGATGA
TTCGGGAAGGTGTGGCCATGCACGCCTTTAACGGTGAACTGTTCGTTCAGGCCACCTGGGATACCA
GTTCGTCGCGGCTTTTCCGGACACAGTTCCGGATGGTCAGCCCGAAGCGCATCAGCAACCCGAACA
ATACCGGCGACAGCCGGAACTGCCGTGCCGGTGTGCAGATTAATGACAGCGGTGCGGCGCTGGGAT
ATTACGTCAGCGAGGACGGGTATCCTGGCTGGATGCCGCAGAAATGGACATGGATACCCCGTGAGT
TACCCGGCGGGCGCGCTTGGCGTAATCATGGTCATAGCTGTTTCCTGTGTGAAATTGTTATCCGCT
CACAATTCCACACAACATACGAGCCGGAAGCATAAAGTGTAAAGCCTGGGGTGCCTAATGAGTGAG
CTAACTCACATTAATTGCGTTGCGCTCACTGCCCGCTTTCCAGTCGGGAAACCTGTCGTGCCAGCT
GCATTAATGAATCGGCCAACGCGCGGGGAGAGGCGGTTTGCGTATTGGGCGCTCTTCCGCTTCCTC
GCTCACTGACTCGCTGCGCTCGGTCGTTCGGCTGCGGCGAGCGGTATCAGCTCACTCAAAGGCGGT
AATACGGTTATCCACAGAATCAGGGGATAACGCAGGAAAGAACATGTGAGCAAAAGGCCAGCAAAA
GGCCAGGAACCGTAAAAAGGCCGCGTTGCTGGCGTTTTTCCATAGGCTCCGCCCCCCTGACGAGCA
TCACAAAAATCGACGCTCAAGTCAGAGGTGGCGAAACCCGACAGGACTATAAAGATACCAGGCGTT
TCCCCCTGGAAGCTCCCTCGTGCGCTCTCCTGTTCCGACCCTGCCGCTTACCGGATACCTGTCCGC
CTTTCTCCCTTCGGGAAGCGTGGCGCTTTCTCATAGCTCACGCTGTAGGTATCTCAGTTCGGTGTA
GGTCGTTCGCTCCAAGCTGGGCTGTGTGCACGAACCCCCCGTTCAGCCCGACCGCTGCGCCTTATC
CGGTAACTATCGTCTTGAGTCCAACCCGGTAAGACACGACTTATCGCCACTGGCAGCAGCCACTGG
TAACAGGATTAGCAGAGCGAGGTATGTAGGCGGTGCTACAGAGTTCTTGAAGTGGTGGCCTAACTA
CGGCTACACTAGAAGGACAGTATTTGGTATCTGCGCTCTGCTGAAGCCAGTTACCTTCGGAAAAAG
AGTTGGTAGCTCTTGATCCGGCAAACAAACCACCGCTGGTAGCGGTGGTTTTTTTGTTTGCAAGCA
GCAGATTACGCGCAGAAAAAAAGGATCTCAAGAAGATCCTTTGATCTTTTCTACGGGGTCTGACGC
TCAGTGGAACGAAAACTCACGTTAAGGGATTTTGGTCATGAGATTATCAAAAAGGATCTTCACCTA
GATCCTTTTAAATTAAAAATGAAGTTTTAAATCAATCTAAAGTATATATGAGTAAACTTGGTCTGA
CAGTTACCAATGCTTAATCAGTGAGGCACCTATCTCAGCGATCTGTCTATTTCGTTCATCCATAGT
TGCCTGACTCCCCGTCGTGTAGATAACTACGATACGGGAGGGCTTACCATCTGGCCCCAGTGCTGC
AATGATACCGCGAGACCCACGCTCACCGGCTCCAGATTTATCAGCAATAAACCAGCCAGCCGGAAG
GGCCGAGCGCAGAAGTGGTCCTGCAACTTTATCCGCCTCCATCCAGTCTATTAATTGTTGCCGGGA
AGCTAGAGTAAGTAGTTCGCCAGTTAATAGTTTGCGCAACGTTGTTGCCATTGCTACAGGCATCGT
GGTGTCACGCTCGTCGTTTGGTATGGCTTCATTCAGCTCCGGTTCCCAACGATCAAGGCGAGTTAC
ATGATCCCCCATGTTGTGCAAAAAAGCGGTTAGCTCCTTCGGTCCTCCGATCGTTGTCAGAAGTAA
GTTGGCCGCAGTGTTATCACTCATGGTTATGGCAGCACTGCATAATTCTCTTACTGTCATGCCATC
CGTAAGATGCTTTTCTGTGACTGGTGAGTACTCAACCAAGTCATTCTGAGAATAGTGTATGCGGCG
ACCGAGTTGCTCTTGCCCGGCGTCAATACGGGATAATACCGCGCCACATAGCAGAACTTTAAAAGT
GCTCATCATTGGAAAACGTTCTTCGGGGCGAAAACTCTCAAGGATCTTACCGCTGTTGAGATCCAG
TTCGATGTAACCCACTCGTGCACCCAACTGATCTTCAGCATCTTTTACTTTCACCAGCGTTTCTGG
GTGAGCAAAAACAGGAAGGCAAAATGCCGCAAAAAAGGGAATAAGGGCGACACGGAAATGTTGAAT
ACTCAT
SEQ ID NO: 34 (STX650)
TCGCGCGTTTCGGTGATGACGGTGAAAACCTCTGACACATGCAGCTCCCGGAGACGGTCACAGCTT
GTCTGTAAGCGGATGCCGGGAGCAGACAAGCCCGTCAGGGCGCGTCAGCGGGTGTTGGCGGGTGTC
GGGGCTGGCTTAACTATGCGGCATCAGAGCAGATTGTACTGAGAGTGCACCATATGCGGTGTGAAA
TACCGCACAGATGCGTAAGGAGAAAATACCGCATCAGGCGCCATTCGCCATTCAGGCTGCGCAACT
GTTGGGAAGGGCGATCGGTGCGGGCCTCTTCGCTATTACGCCAGCTGGCGAAAGGGGGATGTGCTG
CAAGGCGATTAAGTTGGGTAACGCCAGGGTTTTCCCAGTCACGACGTTGTAAAACGACGGCCAGTG
AATTCTCTGCGCGCTCGCTCGCTCACTGAGGCCGGGCGACCAAAGGTCGCCCGACGCCCGGGCTTT
GCCCGGGCGGCCTCAGTGAGCGAGCGAGCGCGCAGAGGATCCTCTACGCCATATTATCCACAGTCC
AACGGCCAGGCGGAGGCTAGTAACAAGGTTATCCTCGGCATCCTCCGCAGGTACCATACGCGTTGA
CATTGATTATTGACTAGTTATTAATAGTAATCAATTACGGGGTCATTAGTTCATAGCCCATATATG
GAGTTCCGCGTTACATAACTTACGGTAAATGGCCCGCCTGGCTGACCGCCCAACGACCCCCGCCCA
TTGACGTCAATAATGACGTATGTTCCCATAGTAACGCCAATAGGGACTTTCCATTGACGTCAATGG
GTGGAGTATTTACGGTAAACTGCCCACTTGGCAGTACATCAAGTGTATCATATGCCAAGTACGCCC
CCTATTGACGTCAATGACGGTAAATGGCCCGCCTGGCATTATGCCCAGTACATGACCTTATGGGAC
TTTCCTACTTGGCAGTACATCTACGTATTAGTCATCGCTATTACCATGGTGATGCGGTTTTGGCAG
TACATCAATGGGCGTGGATAGCGGTTTGACTCACGGGGATTTCCAAGTCTCCACCCCATTGACGTC
AATGGGAGTTTGTTTTGGCACCAAAATCAACGGGACTTTCCAAAATGTCGTAACAACTCCGCCCCA
TTGACGCAAATGGGCGGTAGGCGTGTACGGTGGGAGGTCTATATAAGCAGAGCTCGTTTAGTGAAC
CGTCAGATCGCCTGGAGACGCCATCCACGCTGTTTTGACCTCCATAGTAGACACCGGGACCGATCC
AGCCTCCGGACTCTAGAGGATCGAACCCTTAAGCCGCCACCATGGTGAGCAAGGGCGAGGAGCTGT
TCACCGGGGTGGTGCCCATCCTGGTCGAGCTGGACGGCGACGTAAACGGCCACAAGTTCAGCGTGT
CCGGCGAGGGCGAGGGCGATGCCACCTACGGCAAGCTGACCCTGAAGTTCATCTGCACCACCGGCA
AGCTGCCCGTGCCCTGGCCCACCCTCGTGACCACCCTGACCTACGGCGTGCAGTGCTTCAGCCGCT
ACCCCGACCACATGAAGCAGCACGACTTCTTCAAGTCCGCCATGCCCGAAGGCTACGTCCAGGAGC
GCACCATCTTCTTCAAGGACGACGGCAACTACAAGACCCGCGCCGAGGTGAAGTTCGAGGGCGACA
CCCTGGTGAACCGCATCGAGCTGAAGGGCATCGACTTCAAGGAGGACGGCAACATCCTGGGGCACA
AGCTGGAGTACAACTACAACAGCCACAACGTCTATATCATGGCCGACAAGCAGAAGAACGGCATCA
AGGTGAACTTCAAGATCCGCCACAACATCGAGGACGGCAGCGTGCAGCTCGCCGACCACTACCAGC
AGAACACCCCCATCGGCGACGGCCCCGTGCTGCTGCCCGACAACCACTACCTGAGCACCCAGTCCG
CCCTGAGCAAAGACCCCAACGAGAAGCGCGATCACATGGTCCTGCTGGAGTTCGTGACCGCCGCCG
GGATCACTCTCGGCATGGACGAGCTGTACAAGTACTCAGATCTCGAGCTCAAGTGAACCGGTCAGA
CATGATAAGATACATTGATGAGTTTGGACAAACCACAACTAGAATGCAGTGAAAAAAATGCTTTAT
TTGTGAAATTTGTGATGCTATTGCTTTATTTGTAACCATTATAAGCTGCAATAAACAAGTTGGTAC
CCTCACGGACCAGTGCAACATATTCCCAACATCCCGTTGCAGCCTATCATTAAACCTTGGCCGGTC
GCGGCTGCAGAGGAACCCCTAGTGATGGAGTTGGCCACTCCCTCTCTGCGCGCTCGCTCGCTCACT
GAGGCCGCCCGGGCAAAGCCCGGGCGTCGGGCGACCTTTGGTCGCCCGGCCTCAGTGAGCGAGCGA
GCGCGCAGAGAGGGAGTGGCCAAAAGCTTGGCGTAATCATGGTCATAGCTGTTTCCTGTGTGAAAT
TGTTATCCGCTCACAATTCCACACAACATACGAGCCGGAAGCATAAAGTGTAAAGCCTGGGGTGCC
TAATGAGTGAGCTAACTCACATTAATTGCGTTGCGCTCACTGCCCGCTTTCCAGTCGGGAAACCTG
TCGTGCCAGCTGCATTAATGAATCGGCCAACGCGCGGGGAGAGGCGGTTTGCGTATTGGGCGCTCT
TCCGCTTCCTCGCTCACTGACTCGCTGCGCTCGGTCGTTCGGCTGCGGCGAGCGGTATCAGCTCAC
TCAAAGGCGGTAATACGGTTATCCACAGAATCAGGGGATAACGCAGGAAAGAACATGTGAGCAAAA
GGCCAGCAAAAGGCCAGGAACCGTAAAAAGGCCGCGTTGCTGGCGTTTTTCCATAGGCTCCGCCCC
CCTGACGAGCATCACAAAAATCGACGCTCAAGTCAGAGGTGGCGAAACCCGACAGGACTATAAAGA
TACCAGGCGTTTCCCCCTGGAAGCTCCCTCGTGCGCTCTCCTGTTCCGACCCTGCCGCTTACCGGA
TACCTGTCCGCCTTTCTCCCTTCGGGAAGCGTGGCGCTTTCTCATAGCTCACGCTGTAGGTATCTC
AGTTCGGTGTAGGTCGTTCGCTCCAAGCTGGGCTGTGTGCACGAACCCCCCGTTCAGCCCGACCGC
TGCGCCTTATCCGGTAACTATCGTCTTGAGTCCAACCCGGTAAGACACGACTTATCGCCACTGGCA
GCAGCCACTGGTAACAGGATTAGCAGAGCGAGGTATGTAGGCGGTGCTACAGAGTTCTTGAAGTGG
TGGCCTAACTACGGCTACACTAGAAGAACAGTATTTGGTATCTGCGCTCTGCTGAAGCCAGTTACC
TTCGGAAAAAGAGTTGGTAGCTCTTGATCCGGCAAACAAACCACCGCTGGTAGCGGTGGTTTTTTT
GTTTGCAAGCAGCAGATTACGCGCAGAAAAAAAGGATCTCAAGAAGATCCTTTGATCTTTTCTACG
GGGTCTGACGCTCAGTGGAACGAAAACTCACGTTAAGGGATTTTGGTCATGAGATTATCAAAAAGG
ATCTTCACCTAGATCCTTTTAAATTAAAAATGAAGTTTTAAATCAATCTAAAGTATATATGAGTAA
ACTTGGTCTGACAGTTACCAATGCTTAATCAGTGAGGCACCTATCTCAGCGATCTGTCTATTTCGT
TCATCCATAGTTGCCTGACTCCCCGTCGTGTAGATAACTACGATACGGGAGGGCTTACCATCTGGC
CCCAGTGCTGCAATGATACCGCGAGACCCACGCTCACCGGCTCCAGATTTATCAGCAATAAACCAG
CCAGCCGGAAGGGCCGAGCGCAGAAGTGGTCCTGCAACTTTATCCGCCTCCATCCAGTCTATTAAT
TGTTGCCGGGAAGCTAGAGTAAGTAGTTCGCCAGTTAATAGTTTGCGCAACGTTGTTGCCATTGCT
ACAGGCATCGTGGTGTCACGCTCGTCGTTTGGTATGGCTTCATTCAGCTCCGGTTCCCAACGATCA
AGGCGAGTTACATGATCCCCCATGTTGTGCAAAAAAGCGGTTAGCTCCTTCGGTCCTCCGATCGTT
GTCAGAAGTAAGTTGGCCGCAGTGTTATCACTCATGGTTATGGCAGCACTGCATAATTCTCTTACT
GTCATGCCATCCGTAAGATGCTTTTCTGTGACTGGTGAGTACTCAACCAAGTCATTCTGAGAATAG
TGTATGCGGCGACCGAGTTGCTCTTGCCCGGCGTCAATACGGGATAATACCGCGCCACATAGCAGA
ACTTTAAAAGTGCTCATCATTGGAAAACGTTCTTCGGGGCGAAAACTCTCAAGGATCTTACCGCTG
TTGAGATCCAGTTCGATGTAACCCACTCGTGCACCCAACTGATCTTCAGCATCTTTTACTTTCACC
AGCGTTTCTGGGTGAGCAAAAACAGGAAGGCAAAATGCCGCAAAAAAGGGAATAAGGGCGACACGG
AAATGTTGAATACTCATACTCTTCCTTTTTCAATATTATTGAAGCATTTATCAGGGTTATTGTCTC
ATGAGCGGATACATATTTGAATGTATTTAGAAAAATAAACAAATAGGGGTTCCGCGCACATTTCCC
CGAAAAGTGCCACCTGACGTCTAAGAAACCATTATTATCATGACATTAACCTATAAAAATAGGCGT
ATCACGAGGCCCTTTCGTC
SEQ ID NO: 35 (STXC002)
ggtacccaactccatgcttaacagtccccaggtacagcccaccctgcgtcgcaaccaggaacagct
ctacagcttcctggagcgccactcgccctacttccgcagccacagtgcgcagattaggagcgccac
ttctttttgtcacttgaaaaacatgtaaaaataatgtactaggagacactttcaataaaggcaaat
gtttttatttgtacactctcgggtgattatttaccccccacccttgccgtctgcgccgtttaaaaa
tcaaaggggttctgccgcgcatcgctatgcgccactggcagggacacgttgcgatactggtgttta
gtgctccacttaaactcaggcacaaccatccgcggcagctcggtgaagttttcactccacaggctg
cgcaccatcaccaacgcgtttagcaggtcgggcgccgatatcttgaagtcgcagttggggcctccg
ccctgcgcgcgcgagttgcgatacacagggttgcagcactggaacactatcagcgccgggtggtgc
acgctggccagcacgctcttgtcggagatcagatccgcgtccaggtcctccgcgttgctcagggcg
aacggagtcaactttggtagctgccttcccaaaaagggtgcatgcccaggctttgagttgcactcg
caccgtagtggcatcagaaggtgaccgtgcccggtctgggcgttaggatacagcgcctgcatgaaa
gccttgatctgcttaaaagccacctgagcctttgcgccttcagagaagaacatgccgcaagacttg
ccggaaaactgattggccggacaggccgcgtcatgcacgcagcaccttgcgtcggtgttggagatc
tgcaccacatttcggccccaccggttcttcacgatcttggccttgctagactgctccttcagcgcg
cgctgcccgttttcgctcgtcacatccatttcaatcacgtgctccttatttatcataatgctcccg
tgtagacacttaagctcgccttcgatctcagcgcagcggtgcagccacaacgcgcagcccgtgggc
tcgtggtgcttgtaggttacctctgcaaacgactgcaggtacgcctgcaggaatcgccccatcatc
gtcacaaaggtcttgttgctggtgaaggtcagctgcaacccgcggtgctcctcgtttagccaggtc
ttgcatacggccgccagagcttccacttggtcaggcagtagcttgaagtttgcctttagatcgtta
tccacgtggtacttgtccatcaacgcgcgcgcagcctccatgcccttctcccacgcagacacgatc
ggcaggctcagcgggtttatcaccgtgctttcactttccgcttcactggactcttccttttcctct
tgcgtccgcataccccgcgccactgggtcgtcttcattcagccgccgcaccgtgcgcttacctccc
ttgccgtgcttgattagcaccggtgggttgctgaaacccaccatttgtagcgccacatcttctctt
tcttcctcgctgtccacgatcacctctggggatggcgggcgctcgggcttgggagaggggcgcttc
tttttctttttggacgcaatggccaaatccgccgtcgaggtcgatggccgcgggctgggtgtgcgc
ggcaccagcgcatcttgtgacgagtcttcttcgtcctcggactcgagacgccgcctcagccgcttt
tttgggggcgcgcggggaggcggcggcgacggcgacggggacgacacgtcctccatggttggtgga
cgtcgcgccgcaccgcgtccgcgctcgggggtggtttcgcgctgctcctcttcccgactggccatt
tccttctcctataggcagaaaaagatcatggagtcagtcgagaaggaggacagcctaaccgccccc
tttgagttcgccaccaccgcctccaccgatgccgccaacgcgcctaccaccttccccgtcgaggca
cccccgcttgaggaggaggaagtgattatcgagcaggacccaggttttgtaagcgaagacgacgag
gatcgctcagtaccaacagaggataaaaagcaagaccaggacgacgcagaggcaaacgaggaacaa
gtcgggcggggggaccaaaggcatggcgactacctagatgtgggagacgacgtgctgttgaagcat
ctgcagcgccagtgcgccattatctgcgacgcgttgcaagagcgcagcgatgtgcccctcgccata
gcggatgtcagccttgcctacgaacgccacctgttctcaccgcgcgtaccccccaaacgccaagaa
aacggcacatgcgagcccaacccgcgcctcaacttctaccccgtatttgccgtgccagaggtgctt
gccacctatcacatctttttccaaaactgcaagatacccctatcctgccgtgccaaccgcagccga
gcggacaagcagctggccttgcggcagggcgctgtcatacctgatatcgcctcgctcgacgaagtg
ccaaaaatctttgagggtcttggacgcgacgagaaacgcgcggcaaacgctctgcaacaagaaaac
agcgaaaatgaaagtcactgtggagtgctggtggaacttgagggtgacaacgcgcgcctagccgtg
ctgaaacgcagcatcgaggtcacccactttgcctacccggcacttaacctaccccccaaggttatg
agcacagtcatgagcgagctgatcgtgcgccgtgcacgacccctggagagggatgcaaacttgcaa
gaacaaaccgaggagggcctacccgcagttggcgatgagcagctggcgcgctggcttgagacgcgc
gagcctgccgacttggaggagcgacgcaagctaatgatggccgcagtgcttgttaccgtggagctt
gagtgcatgcagcggttctttgctgacccggagatgcagcgcaagctagaggaaacgttgcactac
acctttcgccagggctacgtgcgccaggcctgcaaaatttccaacgtggagctctgcaacctggtc
tcctaccttggaattttgcacgaaaaccgcctcgggcaaaacgtgcttcattccacgctcaagggc
gaggcgcgccgcgactacgtccgcgactgcgtttacttatttctgtgctacacctggcaaacggcc
atgggcgtgtggcagcaatgcctggaggagcgcaacctaaaggagctgcagaagctgctaaagcaa
aacttgaaggacctatggacggccttcaacgagcgctccgtggccgcgcacctggcggacattatc
ttccccgaacgcctgcttaaaaccctgcaacagggtctgccagacttcaccagtcaaagcatgttg
caaaactttaggaactttatcctagagcgttcaggaattctgcccgccacctgctgtgcgcttcct
agcgactttgtgcccattaagtaccgtgaatgccctccgccgctttggggtcactgctaccttctg
cagctagccaactaccttgcctaccactccgacatcatggaagacgtgagcggtgacggcctactg
gagtgtcactgtcgctgcaacctatgcaccccgcaccgctccctggtctgcaattcgcaactgctt
agcgaaagtcaaattatcggtacctttgagctgcagggtccctcgcctgacgaaaagtccgcggct
ccggggttgaaactcactccggggctgtggacgtcggcttaccttcgcaaatttgtacctgaggac
taccacgcccacgagattaggttctacgaagaccaatcccgcccgccaaatgcggagcttaccgcc
tgcgtcattacccagggccacatccttggccaattgcaagccatcaacaaagcccgccaagagttt
ctgctacgaaagggacggggggtttacctggacccccagtccggcgaggagctcaacccaatcccc
ccgccgccgcagccctatcagcagccgcgggcccttgcttcccaggatggcacccaaaaagaagct
gcagctgccgccgccgccacccacggacgaggaggaatactgggacagtcaggcagaggaggtttt
ggacgaggaggaggagatgatggaagactgggacagcctagacgaagcttccgaggccgaagaggt
gtcagacgaaacaccgtcaccctcggtcgcattcccctcgccggcgccccagaaattggcaaccgt
tcccagcatcgctacaacctccgctcctcaggcgccgccggcactgcctgttcgccgacccaaccg
tagatgggacaccactggaaccagggccggtaagtctaagcagccgccgccgttagcccaagagca
acaacagcgccaaggctaccgctcgtggcgcgggcacaagaacgccatagttgcttgcttgcaaga
ctgtgggggcaacatctccttcgcccgccgctttcttctctaccatcacggcgtggccttcccccg
taacatcctgcattactaccgtcatctctacagcccctactgcaccggcggcagcggcagcggcag
caacagcagcggtcacacagaagcaaaggcgaccggatagcaagactctgacaaagcccaagaaat
ccacagcggcggcagcagcaggaggaggagcgctgcgtctggcgcccaacgaacccgtatcgaccc
gcgagcttagaaataggatttttcccactctgtatgctatatttcaacaaagcaggggccaagaac
aagagctgaaaataaaaaacaggtctctgcgctccctcacccgcagctgcctgtatcacaaaagcg
aagatcagcttcggcgcacgctggaagacgcggaggctctcttcagcaaatactgcgcgctgactc
ttaaggactagtttcgcgccctttctcaaatttaagcgcgaaaactacgtcatctccagcggccac
acccggcgccagcacctgtcgtcagcgccattatgagcaaggaaattcccacgccctacatgtgga
gttaccagccacaaatgggacttgcggctggagctgcccaagactactcaacccgaataaactaca
tgagcgcgggaccccacatgatatcccgggtcaacggaatccgcgcccaccgaaaccgaattctcc
tcgaacaggcggctattaccaccacacctcgtaataaccttaatccccgtagttggcccgctgccc
tggtgtaccaggaaagtcccgctcccaccactgtggtacttcccagagacgcccaggccgaagttc
agatgactaactcaggggcgcagcttgcgggcggctttcgtcacagggtgcggtcgcccgggcgtt
ttagggcggagtaacttgcatgtattgggaattgtagtttttttaaaatgggaagtgacgtatcgt
gggaaaacggaagtgaagatttgaggaagttgtgggttttttggctttcgtttctgggcgtaggtt
cgcgtgcggttttctgggtgttttttgtggactttaaccgttacgtcattttttagtcctatatat
actcgctctgtacttggccctttttacactgtgactgattgagctggtgccgtgtcgagtggtgtt
ttttaataggtttttttactggtaaggctgactgttatggctgccgctgtggaagcgctgtatgtt
gttctggagcgggagggtgctattttgcctaggcaggagggtttttcaggtgtttatgtgtttttc
tctcctattaattttgttatacctcctatgggggctgtaatgttgtctctacgcctgcgggtatgt
attcccccgggctatttcggtcgctttttagcactgaccgatgttaaccaacctgatgtgtttacc
gagtcttacattatgactccggacatgaccgaggaactgtcggtggtgctttttaatcacggtgac
cagtttttttacggtcacgccggcatggccgtagtccgtcttatgcttataagggttgtttttcct
gttgtaagacaggcttctaatgtttaaatgtttttttttttgttattttattttgtgtttaatgca
ggaacccgcagacatgtttgagagaaaaatggtgtctttttctgtggtggttccggaacttacctg
cctttatctgcatgagcatgactacgatgtgcttgcttttttgcgcgaggctttgcctgatttttt
gagcagcaccttgcattttatatcgccgcccatgcaacaagcttacataggggctacgctggttag
catagctccgagtatgcgtgtcataatcagtgtgggttcttttgtcatggttcctggcggggaagt
ggccgcgctggtccgtgcagacctgcacgattatgttcagctggccctgcgaagggacctacggga
tcgcggtatttttgttaatgttccgcttttgaatcttatacaggtctgtgaggaacctgaattttt
gcaatcatgattcgctgcttgaggctgaaggtggagggcgctctggagcagatttttacaatggcc
ggacttaatattcgggatttgcttagagacatattgataaggtggcgagatgaaaattatttgggc
atggttgaaggtgctggaatgtttatagaggagattcaccctgaagggtttagcctttacgtccac
ttggacgtgagggcagtttgccttttggaagccattgtgcaacatcttacaaatgccattatctgt
tctttggctgtagagtttgaccacgccaccggaggggagcgcgttcacttaatagatcttcatttt
gaggttttggataatcttttggaataaaaaaaaaaaaacatggttcttccagctcttcccgctcct
cccgtgtgtgactcgcagaacgaatgtgtaggttggctgggtgtggcttattctgcggtggtggat
gttatcagggcagcggcgcatgaaggagtttacatagaacccgaagccagggggcgcctggatgct
ttgagagagtggatatactacaactactacacagagcgagctaagcgacgagaccggagacgcaga
tctgtttgtcacgcccgcacctggttttgcttcaggaaatatgactacgtccggcgttccatttgg
catgacactacgaccaacacgatctcggttgtctcggcgcactccgtacagtagggatcgcctacc
tccttttgagacagagacccgcgctaccatactggaggatcatccgctgctgcccgaatgtaacac
tttgacaatgcacaacgtgagttacgtgcgaggtcttccctgcagtgtgggatttacgctgattca
ggaatgggttgttccctgggatatggttctgacgcgggaggagcttgtaatcctgaggaagtgtat
gcacgtgtgcctgtgttgtgccaacattgatatcatgacgagcatgatgatccatggttacgagtc
ctgggctctccactgtcattgttccagtcccggttccctgcagtgcatagccggcgggcaggtttt
ggccagctggtttaggatggtggtggatggcgccatgtttaatcagaggtttatatggtaccggga
ggtggtgaattacaacatgccaaaagaggtaatgtttatgtccagcgtgtttatgaggggtcgcca
cttaatctacctgcgcttgtggtatgatggccacgtgggttctgtggtccccgccatgagctttgg
atacagcgccttgcactgtgggattttgaacaatattgtggtgctgtgctgcagttactgtgctga
tttaagtgagatcagggtgcgctgctgtgcccggaggacaaggcgtctcatgctgcgggcggtgcg
aatcatcgctgaggagaccactgccatgttgtattcctgcaggacggagcggcggcggcagcagtt
tattcgcgcgctgctgcagcaccaccgccctatcctgatgcacgattatgactctacccccatgta
ggcgtggacttccccttcgccgcccgttgagcaaccgcaagttggacagcagcctgtggctcagca
gctggacagcgacatgaacttaagcgagctgcccggggagtttattaatatcactgatgagcgttt
ggctcgacaggaaaccgtgtggaatataacacctaagaatatgtctgttacccatgatatgatgct
ttttaaggccagccggggagaaaggactgtgtactctgtgtgttgggagggaggtggcaggttgaa
tactagggttctgtgagtttgattaaggtacggtgatcaatataagctatgtggtggtggggctat
actactgaatgaaaaatgacttgaaattttctgcaattgaaaaataaacacgttgaaacataacat
gcaacaggttcacgattctttattcctgggcaatgtaggagaaggtgtaagagttggtagcaaaag
tttcagtggtgtattttccactttcccaggaccatgtaaaagacatagagtaagtgcttacctcgc
tagtttctgtggattcactagaatcgatgtcgacgtttaaaccatatgatcagctcactcaaaggc
ggtaatacggttatccacagaatcaggggataacgcaggaaagaacatgtgagcaaaaggccagca
aaaggccaggaaccgtaaaaaggccgcgttgctggcgtttttccataggctccgcccccctgacga
gcatcacaaaaatcgacgctcaagtcagaggtggcgaaacccgacaggactataaagataccaggc
gtttccccctggaagctccctcgtgcgctctcctgttccgaccctgccgcttaccggatacctgtc
cgcctttctcccttcgggaagcgtggcgctttctcatagctcacgctgtaggtatctcagttcggt
gtaggtcgttcgctccaagctgggctgtgtgcacgaaccccccgttcagcccgaccgctgcgcctt
atccggtaactatcgtcttgagtccaacccggtaagacacgacttatcgccactggcagcagccac
tggtaacaggattagcagagcgaggtatgtaggcggtgctacagagttcttgaagtggtggcctaa
ctacggctacactagaagaacagtatttggtatctgcgctctgctgaagccagttaccttcggaaa
aagagttggtagctcttgatccggcaaacaaaccaccgctggtagcggtggtttttttgtttgcaa
gcagcagattacgcgcagaaaaaaaggatctcaagaagatcctttgatcttttctacggggtctga
cgctcagtggaacgaaaactcacgttaagggattttggtcatgagattatcaaaaaggatcttcac
ctagatccttttaaattaaaaatgaagttttaaatcaatctaaagtatatatgagtaaacttggtc
tgacagttaccaatgcttaatcagtgaggcacctatctcagcgatctgtctatttcgttcatccat
agttgcctgactccccgtcgtgtagataactacgatacgggagggcttaccatctggccccagtgc
tgcaatgataccgcgagacccacgctcaccggctccagatttatcagcaataaaccagccagccgg
aagggccgagcgcagaagtggtcctgcaactttatccgcctccatccagtctattaattgttgccg
ggaagctagagtaagtagttcgccagttaatagtttgcgcaacgttgttgccattgctacaggcat
cgtggtgtcacgctcgtcgtttggtatggcttcattcagctccggttcccaacgatcaaggcgagt
tacatgatcccccatgttgtgcaaaaaagcggttagctccttcggtcctccgatcgttgtcagaag
taagttggccgcagtgttatcactcatggttatggcagcactgcataattctcttactgtcatgcc
atccgtaagatgcttttctgtgactggtgagtactcaaccaagtcattctgagaatagtgtatgcg
gcgaccgagttgctcttgcccggcgtcaatacgggataataccgcgccacatagcagaactttaaa
agtgctcatcattggaaaacgttcttcggggcgaaaactctcaaggatcttaccgctgttgagatc
cagttcgatgtaacccactcgtgcacccaactgatcttcagcatcttttactttcaccagcgtttc
tgggtgagcaaaaacaggaaggcaaaatgccgcaaaaaagggaataagggcgacacggaaatgttg
aatactcatactcttcctttttcaatattattgaagcatttatcagggttattgtctcatgagcgg
atacatatttgaatgtatttagaaaaataaacaaataggggttccgcgcacatttccccgaaaagt
gccacctaaattgtaagcgttaatattttgttaaaattcgcgttaaatttttgttaaatcagctca
ttttttaaccaataggccgaaatcggcaaaatcccttataaatcaaaagaatagaccgagataggg
ttgagtgttgttccagtttggaacaagagtccactattaaagaacgtggactccaacgtcaaaggg
cgaaaaaccgtctatcagggcgatggcccactacgtgaaccatcaccctaatcaagttttttgggg
tcgaggtgccgtaaagcactaaatcggaaccctaaagggagcccccgatttagagcttgacgggga
aagccggcgaacgtggcgagaaaggaagggaagaaagcgaaaggagcgggcgctagggcgctggca
agtgtagcggtcacgctgcgcgtaaccaccacacccgccgcgcttaatgcgccgctacagggcgcg
atggatcc

### Exemplary Construct 1/Rep/Cap Features

| Name | type | location |
|---|---|---|
| Rep68 CDS2 | misc_feature | 4012 . . . 4036 |
| Rabbit beta globin | intronmisc_feature | 878 . . . 1230 |
| Rep40 CDS2 | misc_feature | 4012 . . . 4036 |
| 3′SS | misc_feature | 2760 . . . 2806 |
| VP1 | misc_feature | 3987 . . . 6194 |
| EGFR | misc_feature | 1312 . . . 2030 |
| VP3 | misc_feature | 4593 . . . 6194 |
| loxP | misc_feature | 1231 . . . 1264 |
| Rep52 | misc_feature | 848 . . . 877 |
| Rep40 CDS1 | misc_feature | 848 . . . 877 |
| Rep78 | misc_feature | 176 . . . 877 |
| Rep68 CDS1 | misc_feature | 176 . . . 877 |
| 5 prime terminus of | RNA1misc_feature | 142 . . . 151 |
| TATA box P5 | misc_feature | 110 . . . 115 |
| TATA box p19 | misc_feature | 698 . . . 705 |
| 5 prime terminus of | RNA2misc_feature | 728 . . . 737 |
| 5 prime terminus of | RNA3misc_feature | 3637 . . . 3644 |
| TATA box of P40 | misc_feature | 3606 . . . 3614 |
| polyA sequence | misc_feature | 6208 . . . 6213 |
| Rep52 | misc_feature | 2807 . . . 3970 |
| Rep40 CDS1 | misc_feature | 2807 . . . 3690 |
| Rep78 | misc_feature | 2807 . . . 3970 |
| Rep68 CDS1 | misc_feature | 2807 . . . 3690 |
| AAV 3′UTR | misc_feature | 6195 . . . 6318 |
| 3′SS | misc_feature | 1265 . . . 1311 |
| Rabbit beta globin | polyAmisc_feature | 2032 . . . 2552 |
| rabbit beta globin | intronmisc_feature | 2587 . . . 2806 |
| VP (CAP) proteins | misc_feature | 3987 . . . 6194 |
| pCRII Topo | misc_feature | 6319 . . . 9837 |
| M13-fwd | primer_bind | rev: 6415 . . . 6432 |
| M13-rev | primer_bind | 9707 . . . 9727 |
| T7 | primer_bind | rev: 6389 . . . 6416 |
| SP6 | primer_bind | 9733 . . . 9758 |
| ColE1 origin | rep_origin | 8657 . . . 9339 |
| LacO | misc_binding | 9679 . . . 9701 |
| LoxP | misc_recomb | rev: 1231 . . . 1264 |
| LoxP | misc_recomb | rev: 2553 . . . 2586 |
| Kan/neoR | CDS | 7223 . . . 8014 |

### 8. EQUIVALENTS AND INCORPORATION BY REFERENCE

All references cited herein are incorporated by reference to the same extent as if each individual publication, database entry (e.g. Genbank sequences or GeneID entries), patent application, or patent, was specifically and individually indicated to be incorporated by reference in its entirety, for all purposes. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

While the invention has been particularly shown and described with reference to a preferred embodiment and various alternate embodiments, it will be understood by persons skilled in the relevant art that various changes in form and details can be made therein without departing from the spirit and scope of the invention.

1-157. (canceled)

158. A polynucleotide, comprising, from 5′ to 3′:
a first sequence comprising:
a first promoter sequence operably linked to a first part of an AAV Rep coding sequence,
a 5′ splice site,
a first intron,
an excisable element comprising, from 5′ to 3′, a first recombination site, a first 3′ splice site, a coding sequence comprising a stop signaling sequence, and a second recombination site,
a second intron, and
a second 3′ splice site; and
a second sequence comprising a second part of the AAV Rep coding sequence, wherein the first promoter is not operably linked to the second sequence in the absence of a recombination event between the first recombination site and the second recombination site and is operably linked to the second sequence following a recombination event between the first recombination site and the second recombination site.

159. The polynucleotide of claim 158, comprising a third sequence comprising an AAV Cap coding sequence, wherein the second sequence comprises a second promoter that is operably linked to the third sequence.

160. The polynucleotide of claim 158, wherein the 5′ splice site is a rabbit beta globin 5′ splice site, and the first 3′ splice site and the second 3′ splice site are each a rabbit beta globin 3′ splice site.

161. The polynucleotide of claim 158, wherein the first recombination site is a first lox sequence and the second recombination site is a second lox sequence.

162. The polynucleotide of claim 161, wherein the first lox sequence is a first loxP sequence and the second lox sequence is a second loxP sequence.

163. The polynucleotide of claim 158, wherein the first recombination site is a first flippase recognition target (FRT) site and the second recombination site is a second FRT site.

164. The polynucleotide of claim 158, wherein the stop signaling sequence is a termination sequence of the coding sequence comprising a stop signaling sequence or is a polyA sequence.

165. The polynucleotide of claim 158, wherein the coding sequence comprising a stop signaling sequence encodes a detectable marker or a selectable marker.

166. The polynucleotide of claim 158, wherein the coding sequence comprising a stop signaling sequence encodes a detectable marker comprising a luminescent marker or a fluorescent marker.

167. The polynucleotide of claim 158, wherein the excisable element is excisable by a recombinase capable of inducing the recombination event between the first recombination site and the second recombination site.

168. The polynucleotide of claim 167, wherein the recombinase is a Cre recombinase or a Flippase recombinase.

169. The polynucleotide of claim 167, wherein the recombinase is an inducible recombinase.

170. The polynucleotide of claim 169, wherein the inducible recombinase is a Cre recombinase fused to an estrogen response (ER) element for translocating to the nucleus in the presence of a triggering agent that is tamoxifen.

171. The polynucleotide of claim 158, wherein the AAV Rep coding sequence encodes Rep polypeptides comprising Rep78, Rep68, Rep52, and Rep40.

172. The polynucleotide of claim 159, wherein the AAV Cap coding sequence encodes AAV capsid proteins comprising VP1, VP2, and VP3.

173. The polynucleotide of claim 158, comprising a fourth sequence encoding for a selectable marker under the control of a constitutive promoter.

174. The polynucleotide of claim 173, wherein the selectable marker is a mammalian cell selection element.

175. The polynucleotide of claim 174, wherein the mammalian cell selection element is an auxotrophic selection element or an antibiotic resistance protein.

176. The polynucleotide of claim 159, wherein:
the AAV Rep coding sequence encodes Rep polypeptides comprising Rep78, Rep68, Rep52, and Rep40; and
the AAV Cap coding sequence encodes AAV capsid proteins comprising VP1, VP2, and VP3.

177. The polynucleotide of claim 176, wherein the excisable element is excisable by a recombinase capable of inducing the recombination event between the first recombination site and the second recombination site, wherein the recombinase is a Cre recombinase fused to an estrogen response (ER) element for translocating to the nucleus in the presence of a triggering agent that is tamoxifen.

178. A polynucleotide, comprising:
a first sequence comprising an inducible promoter operably linked to a first excisable element comprising a sequence encoding a recombinase that is flanked by a first recombination site and a second recombination site;
a second sequence comprising a first constitutive promoter operably linked to a sequence encoding an activator for inducing the inducible promoter in the presence of a first triggering agent; and
a third sequence encoding one or more AAV helper proteins, wherein the inducible promoter is not operably linked to the third sequence in the absence of a recombination event between the first recombination site and the second recombination site and is operably linked to the third sequence following a recombination event between the first recombination site and the second recombination site.

179. The polynucleotide of claim 178, wherein the third sequence encoding one or more AAV helper proteins comprises a bicistronic open reading frame encoding E2a and E4.

180. The polynucleotide of claim 178, wherein the inducible promoter is a tetracycline-response promoter element (TRE).

181. The polynucleotide of claim 180, wherein the TRE comprises Tet operator (tetO) sequence concatemers fused to a minimal promoter.

182. The polynucleotide of claim 180, wherein the activator is reverse tetracycline-controlled transactivator (rTA) comprising a Tet Repressor binding protein (TetR) fused to a VP16 transactivation domain, and the first triggering agent is tetracycline or doxycycline.

183. The polynucleotide of claim 178, wherein the recombinase is an inducible recombinase.

184. The polynucleotide of claim 183, wherein the inducible recombinase is a Cre recombinase fused to an estrogen response (ER) element for translocating to the nucleus in the presence of a second triggering agent that is tamoxifen.

185. The polynucleotide of claim 178, comprising a fourth sequence comprising:
a first part of a second constitutive promoter and a second part of the second constitutive promoter separated by a second excisable element comprising a third recombination site and a fourth recombination site flanking a stuffer sequence; and
a VA-RNA coding sequence that is not operably linked to the second constitutive promoter in the absence of a recombination event between the third recombination site and the fourth recombination site and is operably linked to the second constitutive promoter following a recombination event between the third recombination site and the fourth recombination site.

186. The polynucleotide of claim 178, comprising a fifth sequence encoding a selectable marker under the control of a third constitutive promoter.

187. The polynucleotide of claim 178, wherein:
the inducible promoter is a TRE comprising tetO sequence concatemers fused to a minimal promoter;
the activator is rTA comprising a TetR fused to a VP16 transactivation domain;
the first triggering agent is tetracycline or doxycycline; and
the recombinase is a Cre recombinase fused to an ER element for translocating to the nucleus in the presence of a triggering agent that is tamoxifen.

188. The polynucleotide of claim 187, comprising a fourth sequence comprising:
a first part of a second constitutive promoter and a second part of the second constitutive promoter separated by a second excisable element comprising a third recombination site and a fourth recombination site flanking a stuffer sequence; and
a VA-RNA coding sequence that is not operably linked to the second constitutive promoter in the absence of a recombination event between the third recombination site and the fourth recombination site and is operably linked to the second constitutive promoter following a recombination event between the third recombination site and the fourth recombination site, wherein the VA-RNA coding sequence encodes a transcriptionally dead VA-RNA.

189. A system for inducible AAV production, comprising:
the polynucleotide of claim 159; and
the polynucleotide of claim 178.

190. The system of claim 189, further comprising a payload polynucleotide comprising a promoter operably linked to a payload sequence.

191. A system for inducible AAV production, comprising:
the AAV Rep/Cap polynucleotide of claim 177; and
the inducible helper polynucleotide of claim 187.

192. The system of claim 191, further comprising a payload polynucleotide comprising a promoter operably linked to a payload sequence.


SEQUENCE LISTING (41 sequences)

SEQ ID NO: 1 - DNA, 353 bp, synthetic construct
  misc_feature 1..353: note = source = /note="Description of Artificial Sequence: Synthetic polynucleotide"
  source 1..353: mol_type = other DNA
gtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaac

SEQ ID NO: 2 - DNA, 1356 bp, synthetic construct
  misc_feature 1..1356: note = source = /note="Description of Artificial Sequence: Synthetic polynucleotide"
  source 1..1356: mol_type = other DNA
ataacttcgtataatgtatgctatacgaagttatcgggcccctctgctaaccatgttcatgccttcttctttttcctacagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcataacttcgtataatgtatgctatacgaagttat

SEQ ID NO: 3 - DNA, 220 bp, synthetic construct
  misc_feature 1..220: note = source = /note="Description of Artificial Sequence: Synthetic polynucleotide"
  source 1..220: mol_type = other DNA
aattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctttttcctacag

SEQ ID NO: 4 - DNA, 534 bp, synthetic construct
  misc_feature 1..534: note = source = /note="Description of Artificial Sequence: Synthetic polynucleotide"
  source 1..534: mol_type = other DNA
atgagagggtcagatccggcagcgttgaagcgggctaggaacactgaggcagcaaggcgctctcgagcaaggaagttgcaacggatgaaacaattggaagacaaagttgaagagctgctttcaaaaaactaccaccttgaaaatgaagtcgcgaggctgaagaaattggtcggatctgctggcagcgcagcggggagcggtgagtttatggtcagacctctcaactgtattgtcgctgtctcacagaacatgggtatcggaaagaacggtgacttgccgtggccgccactgcggaatgagttcaaatactttcagcgcatgacgaccaccagcagtgtggagggtaagcaaaatcttgtcataatgggtcgcaagacttggttttctattccagagaaaaacagaccgcttaaagataggattaacatcgtgttgagccgggaactgaaagagccaccaaggggagcacattttttggctaagtccttggatgacgccctgcgactgatagagcaaccagaacttgcttagtaa

SEQ ID NO: 5 - DNA, 453 bp, synthetic construct
  misc_feature 1..453: note = source = /note="Description of Artificial Sequence: Synthetic polynucleotide"
  source 1..453: mol_type = other DNA
atgcgcggttccgacccagcagctttgaaacgagcacgaaacacggaagcagcccgcaggagtcgagcgagaaaacttcagcggatgaagcagcttgaagataaagtcgaggaattgcttagcaagaattatcacctcgagaatgaagtggcgcgactgaaaaaacttgtaggttctgctgggagcgcagccggaagcggcgagttctcaaaagttgacatggtgtggatcgtgggtggaagttctgtctatcaagaggcgatgaatcagcctggccacctcagactgtttgttacaaggatcatgcaggagttcgagtctgacacgttttttccagagatcgacctggggaaatataaactcctcccagagtacccaggagtgcttagtgaggtccaagaagagaagggaatcaaatataaatttgaagtttacgaaaagaaggattagtaa

SEQ ID NO: 6 - DNA, 9837 bp, synthetic construct
  misc_feature 1..9837: note = source = /note="Description of Artificial Sequence: Synthetic polynucleotide"
  source 1..9837: mol_type = other DNA
ggaggggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacataacttcgtataatgtatgctatacgaagttatcgggcccctctgctaaccatgttcatgccttcttctttttcctacagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcataacttcgtataatgtatgctatacgaagttataattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctttttcctacaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatggctacgtagataagtagcatggcgggttaatcattaactacataagggcgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacggcagtttaaggtttacacctataaaagagagagccgttatcgtctgtttgtggatgtacagagtgatattattgacacgccggggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggcatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgaggagggccaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctatgcatcaagcttggtaccgagctcggatccactagtaacggccgccagtgtgctggaattcgccct

SEQ ID NO: 7 - DNA, 6367 bp, synthetic construct
  misc_feature 1..6367: note = source = /note="Description of Artificial Sequence: Synthetic polynucleotide"
  source 1..6367: mol_type = other DNA
ggcctccacggccactagtaacggccgccagtgtgctggaattcgccctggaggggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacataacttcgtataatgtatgctatacgaagttatcgggcccctctgctaaccatgttcatgccttcttctttttcctacagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcataacttcgtataatgtatgctatacgaagttataattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctttttcctacaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatggctacgtagataagtagcatggcgggttaatcattaactaca

SEQ ID NO: 8 - DNA, 12385 bp, synthetic construct
  misc_feature 1..12385: note = source = /note="Description of Artificial Sequence: Synthetic polynucleotide"
  source 1..12385: mol_type = other DNA
actcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccactagtaacggccgccagtgtgctggaattcgccctggaggggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacataacttcgtataatgtatgctatacgaagttatcgggcccctctgctaaccatgttcatgccttcttctttttcctacagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcataacttcgtataatgtatgctatacgaagttataattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctttttcctacaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatggctacgtagataagtagcatggcgggttaatcattaactacataagggcgaattctgcagatatccatcacactggcggccgctcgagcatgcatctagagctagcgaattcgaatttaaatcggatccgcggccgcaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgatatcgccaccatgaaaacatttaacatttctcaacaggatctagaattagtagaagtagcgacagagaagattacaatgctttatgaggataataaacatcatgtgggagcggcaattcgtacgaaaacaggagaaatcatttcggcagtacatattgaagcgtatataggacgagtaactgtttgtgcagaagccattgcgattggtagtgcagtttcgaatggacaaaaggattttgacacgattgtagctgttagacacccttattctgacgaagtagatagaagtattcgagtggtaagtccttgtggtatgtgtagggagttgatttcagactatgcaccagattgttttgtgttaatagaaatgaatggcaagttagtcaaaactacgattgaagaactcattccactcaaatatacccgaaattaaggtacctcgacaaccttccaaactgagtgcatgacccgcaagcccggtgcctgaaatcaacctctggattacaaaatttgtgaaagattgactggtattcttaactatgttgctccttttacgctatgtggatacgctgctttaatgcctttgtatcatgcgttaactaaacttgtttattgcagcttataatggttacaaataaagcaatagcatcacaaatttcacaaataaagcatttttttcactgcattctagttgtggtttgtccaaactcatcaatgtatcttatcatgtctggaattgactcaaatgatgtcaattagtctatcagaagctatctggtctcccttccgggggacaagacatccctgtttaatatttaaacagcagtgttcccaaactgggttcttatatcccttgctctggtcaaccaggttgcagggtttcctgtcctcacaggaacgaagtccctaaagaaacagtggcagccaggtttagccccggaattgactggattccttttttagggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 9 - DNA, 14766 bp, synthetic construct
  misc_feature 1..14766: note = source = /note="Description of Artificial Sequence: Synthetic polynucleotide"
  source 1..14766: mol_type = other DNA
actcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccactagtccatagagcccaccgcatccccagcatgcctgctattgtcttcccaatcctcccccttgctgtcctgccccaccccaccccctagaatagaatgacacctactcagacaatgcgatgcaatttcctcattttattaggaaaggacagtgggagtggcaccttccagggtcaaggaaggcacgggggaggggcaaacaacagatggctggcaactagaaggcacagctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcatgagacgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatgcactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgtcagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtctctgtctcaaaaggaggtaggcgatccctactgtacggagtgcgccgagacaaccgagatcgtgttggtcgtagtgtcatgccaaatggaacgccggacgtagtcatggttgtggccatattatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcttaaaaatcaaaggggttctgccgcgcatcactatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcgcttgtcgtcatcgtctttgtagtcgggaggcggcggcgacggcgacggggacgacacgtcctccatggttggtggacgtcgcgccgcaccgcgtccgcgctcgggggtggtttcgcgctgctcctcttcccgactggccatggtggccgaggataacttcgtatatggtttcttatacgaagttatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatctgctacgtatcaagctgtggcagggaaaccctctgcctcccccgtgatgtaatacttttgcaaggaatgcgatgaagtagagcccgcagtggccaagtggctttggtccgtctcctccacggatgcccctccacggctagtgggcgcatgtaggcggtgggcgtccgccgcctccagcagcaggtcatagaggggcaccacgttcttgcacttcatgctgtacagatgctccatgcctttgttactcatgtgtcggatgtgggagaggatgaggaggagctgggccagccgctggtgctgctgctgcagggtcaggcctgccttggccatcaggtggatcaaagtgtctgtgatcttgtccaggactcggtggatatggtccttctcttccagagacttcagggtgctggacagaaatgtgtacactccagaattaagcaaaataatagatttgaggcacacaaactcctctccctgcagattcatcatgcggaaccgagatgatgtagccagcagcatgtcgaagatctccaccatgccctctacacattttccctggttcctgtccaagagcaagttaggagcaaacagtagcttcactgggtgctccatggagcgccagacgagaccaatcatcaggatctctagccaggcacattctagaaggtggacctgatcatggagggtcaaatccacaaagcctggcaccctcttcgcccagttgatcatgtgaaccagctccctgtctgccaggttggtcagtaagcccatcatcgaagcttcactgaagggtctggtaggatcatactcggaatagagtatggggggctcagcatccaacaaggcactgaccatctggtcggccgtcagggacaaggccaggctgttcttcttagagcgtttgatcatgagcgggcttggccaaaggttggcagctctcatgtctccagcagatggctcgagatcgccatcttccagcaggcgcaccattgcccctgtttcactatccaggttacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatcctaaaatacacaaacaattagaatcagtagtttaacacattatacacttaaaaattttatatttaccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagtgactcccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacggacttggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcttgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacatggtggctacgtaataacttcgtatatggtttcttatacgaagttatgcggccgctttacgagggtaggaagtggtacggaaagttggtataagacaaaagtgttgtggaattgctccaggcgatctgacggttcactaaacgagctctgcttttataggcgcccaccgtacacgcctaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactcgcggccgcagagaaatgttctggcacctgcacttgcactggggacagcctattttgctagtttgttttgtttcgttttgttttgatggagagcgtatgttagtactatcgattcacacaaaaaaccaacacacagatgtaatgaaaataaagatattttattggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatgtaagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagcggatccgccgccaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctcttctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtgaaccggtcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttagagggcctatttcccatgattccttcatatttgcatatacgatacaaggctgttagagagataattagaattaatttgactgtaaacacaaagatattagtacaaaataataacttcgtataatgtatgctatacgaagttatcagacatgataagatacattgatgagtttggacaaaccacaactagaatgcagtgaaaaaaatgctttatttgtgaaatttgtgatgctattgctttatttgtaaccattataagctgcaataaacaaggtacctcaagcgccgggttttcgcgtcatgcaccacgtccgtgggccctcgggtacttcaacgtcagcagtaactgtaaatccgagccgttcatagaagggcaaattccttggcgctgacgtttcaagaaaggctggcactccggctcgttctgcggcttctactccgggcaataccaccgcggaaccaaggccctttccctgatgatcggggctaacgcccacagtagcgaggaaccaagctggttctttagggcggtgaggggcgaggagtccttccatttgttgctgagccgcgagacgagagccactaagctcagccattcggggaccaatttctgcaaatacagccccggcctcaacgctctccggagtcgtccacactgccactgcagccccgtcgtcggcgacccaaactttaccgatgtccaatcctaccctggtcaaaaaaagttcttgcaattctgtaacccgttcaatatgtctatcaggatcaactgtgtggcgtgtagcgggataatccgcgaaagcggcagccaatgttctcacggccctagggacgtcgtctcgagttgccagtctgacagtaggtttatattctgtcataggtccagggttctcctccacgtctccagcctgcttcagcaggctgaagttagtagctccggatcctttacctccatcaccagcgccaccagtagagtatctggccacagccacctcgtgctgctcgacgtaggtctcatcgtcggcctccttgattctttccagtctgtggtccacgttgtagacgccgggcatcttgaggttcgtagcgggtttcttggatctgtatgtggtctcaaggttgcagatcaggtggcccccgcccacgagcttcagggccatgtcacatgcgccttccaggccgccgtcagcggggtacatcgtctcggtggaggcctcccagccgagtgttttcttctgcatcacagggccgttggctgggaagttcacccctctaaccttgacgttgtagatgaggcagccgtcctggaggctggtgtcctgggtagcggtcagcacgcccccgtcttcgtatgtggtgactctctcccatgtgaagccctcagggaaggactgcttaaagaagtcggggatgcccggagggtgcttgatgaaggttctgctgccgtacatgaagctggtagccaggatgtcgaaggcgaaggggagagggccgccctcgacgaccttgattctcatggtctgggtgccctcgtagggcttgccttcgccctcggatgtgcacttgaagtggtggttgttcacggtgccctccatgtacagcttcatgggcatgttctccttaatcagctcgctcacggtggcggcgaattccgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgcctaccggtggatgtggaatgtgtgcgaggccagaggccacttgtgtagcgccaagtgcccagcggggctgctaaagcgcatgctccagactgccttgggaaaagcgctcccctacccataacttcgtataatgtatgctatacgaagttattttgcagttttaaaattatgttttaaaatggactatcatatgcttaccgtaacttgaaagtatttcgatttcttggctttatatatcttgtggaaaggacgaaacaccgggcactcttccgtgatctggtggataaattcgcaagggtatcatggcggacgaccgggattcgaaccccggatccggccgtccgccgtgatccatgcggttaccgcccgcgtgtcgaacccaggtgtgcgacgtcagacaacgggggagcgctcctttttgggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 10 - DNA, 13743 bp, synthetic construct
  misc_feature 1..13743: note = source = /note="Description of Artificial Sequence: Synthetic polynucleotide"
  source 1..13743: mol_type = other DNA
actcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccactagtccatagagcccaccgcatccccagcatgcctgctattgtcttcccaatcctcccccttgctgtcctgccccaccccaccccctagaatagaatgacacctactcagacaatgcgatgcaatttcctcattttattaggaaaggacagtgggagtggcaccttccagggtcaaggaaggcacgggggaggggcaaacaacagatggctggcaactagaaggcacagctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcatgagacgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatgcactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgtcagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtctctgtctcaaaaggaggtaggcgatccctactgtacggagtgcgccgagacaaccgagatcgtgttggtcgtagtgtcatgccaaatggaacgccggacgtagtcatggttgtggccatattatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcttaaaaatcaaaggggttctgccgcgcatcactatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcgcttgtcgtcatcgtctttgtagtcgggaggcggcggcgacggcgacggggacgacacgtcctccatggttggtggacgtcgcgccgcaccgcgtccgcgctcgggggtggtttcgcgctgctcctcttcccgactggccatggtggccgaggataacttcgtatatggtttcttatacgaagttatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatctgctacgtatcaagctgtggcagggaaaccctctgcctcccccgtgatgtaatacttttgcaaggaatgcgatgaagtagagcccgcagtggccaagtggctttggtccgtctcctccacggatgcccctccacggctagtgggcgcatgtaggcggtgggcgtccgccgcctccagcagcaggtcatagaggggcaccacgttcttgcacttcatgctgtacagatgctccatgcctttgttactcatgtgtcggatgtgggagaggatgaggaggagctgggccagccgctggtgctgctgctgcagggtcaggcctgccttggccatcaggtggatcaaagtgtctgtgatcttgtccaggactcggtggatatggtccttctcttccagagacttcagggtgctggacagaaatgtgtacactccagaattaagcaaaataatagatttgaggcacacaaactcctctccctgcagattcatcatgcggaaccgagatgatgtagccagcagcatgtcgaagatctccaccatgccctctacacattttccctggttcctgtccaagagcaagttaggagcaaacagtagcttcactgggtgctccatggagcgccagacgagaccaatcatcaggatctctagccaggcacattctagaaggtggacctgatcatggagggtcaaatccacaaagcctggcaccctcttcgcccagttgatcatgtgaaccagctccctgtctgccaggttggtcagtaagcccatcatcgaagcttcactgaagggtctggtaggatcatactcggaatagagtatggggggctcagcatccaacaaggcactgaccatctggtcggccgtcagggacaaggccaggctgttcttcttagagcgtttgatcatgagcgggcttggccaaaggttggcagctctcatgtctccagcagatggctcgagatcgccatcttccagcaggcgcaccattgcccctgtttcactatccaggttacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatcctaaaatacacaaacaattagaatcagtagtttaacacattatacacttaaaaattttatatttaccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagtgactcccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacggacttggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcttgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacatggtggctacgtaataacttcgtatatggtttcttatacgaagttatgcggccgctttacgagggtaggaagtggtacggaaagttggtataagacaaaagtgttgtggaattgctccaggcgatctgacggttcactaaacgagctctgcttttataggcgcccaccgtacacgcctaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactcgcggccgcagagaaatgttctggcacctgcacttgcactggggacagcctattttgctagtttgttttgtttcgttttgttttgatggagagcgtatgttagtactatcgattcacacaaaaaaccaacacacagatgtaatgaaaataaagatattttattggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatgtaagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagcggatccgccgccaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctcttctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggggatccggagctactaacttcagcctgctgaagcaggctggagacgtggaggagaaccctggacctatgacagaatataaacctactgtcagactggcaactcgagacgacgtccctagggccgtgagaacattggctgccgctttcgcggattatcccgctacacgccacacagttgatcctgatagacatattgaacgggttacagaattgcaagaactttttttgaccagggtaggattggacatcggtaaagtttgggtcgccgacgacggggctgcagtggcagtgtggacgactccggagagcgttgaggccggggctgtatttgcagaaattggtccccgaatggctgagcttagtggctctcgtctcgcggctcagcaacaaatggaaggactcctcgcccctcaccgccctaaagaaccagcttggttcctcgctactgtgggcgttagccccgatcatcagggaaagggccttggttccgcggtggtattgcccggagtagaagccgcagaacgagccggagtgccagcctttcttgaaacgtcagcgccaaggaatttgcccttctatgaacggctcggatttacagttactgctgacgttgaagtacccgagggcccacggacgtggtgcatgacgcgaaaacccggcgcttgagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttagtttaaacgggcccttaattaatcgatgtaggatgttgcccctcctgacgcggtaggagaaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggcccgagactcgaaccgggggtcctgcgactcaacccttggaaaataaccctccggctacagggagcgagccacttaatgctttcgctttccagcctaaccgcttacgccgcgcgcggccagtggccaaaaaagctagcgcagcagccgccgcgcctggaaggaagccaaaaggagcgctcccccgttgtctgacgtcgcacacctgggttcgacacgcgggcggtaaccgcatggatcacggcggacggccggatccggggttcgaaccccggtcgtccgccatgatacccttgcgaatttatccaccagaccacggaagagtgcccgcttacaggctctccttttgcacggtctagagcgtcaacgactgcgcacgcctcaccggccagagcgtcccgaccatggagcactttttgccgctgcgcaacatctggaaccgcgtccgcgactttccgcgcgcctccaccaccgccgccggcatcacctggatgtccaggtacatctacggattacggggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 11 - DNA, 10347 bp, synthetic construct
  misc_feature 1..10347: note = source = /note="Description of Artificial Sequence: Synthetic polynucleotide"
  source 1..10347: mol_type = other DNA
gcctccacggccactagtccatagagcccaccgcatccccagcatgcctgctattgtcttcccaatcctcccccttgctgtcctgccccaccccaccccctagaatagaatgacacctactcagacaatgcgatgcaatttcctcattttattaggaaaggacagtgggagtggcaccttccagggtcaaggaaggcacgggggaggggcaaacaacagatggctggcaactagaaggcacagctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcatgagacgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatgcactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgtcagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtctctgtctcaaaaggaggtaggcgatccctactgtacggagtgcgccgagacaaccgagatcgtgttggtcgtagtgtcatgccaaatggaacgccggacgtagtcatggttgtggccatattatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcttaaaaatcaaaggggttctgccgcgcatcactatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcgcttgtcgtcatcgtctttgtagtcgggaggcggcggcgacggcgacggggacgacacgtcctccatggttggtggacgtcgcgccgcaccgcgtccgcgctcgggggtggtttcgcgctgctcctcttcccgactggccatggtggccgaggataacttcgtatatggtttcttatacgaagttatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatctgctacgtatcaagctgtggcagggaaaccctctgcctcccccgtgatgtaatacttttgcaaggaatgcgatgaagtagagcccgcagtggccaagtggctttggtccgtctcctccacggatgcccctccacggctagtgggcgcatgtaggcggtgggcgtccgccgcctccagcagcaggtcatagaggggcaccacgttcttgcacttcatgctgtacagatgctccatgcctttgttactcatgtgtcggatgtgggagaggatgaggaggagctgggccagccgctggtgctgctgctgcagggtcaggcctgccttggccatcaggtggatcaaagtgtctgtgatcttgtccaggactcggtggatatggtccttctcttccagagacttcagggtgctggacagaaatgtgtacactccagaattaagcaaaataatagatttgaggcacacaaactcctctccctgcagattcatcatgcggaaccgagatgatgtagccagcagcatgtcgaagatctccaccatgccctctacacattttccctggttcctgtccaagagcaagttaggagcaaacagtagcttcactgggtgctccatggagcgccagacgagaccaatcatcaggatctctagccaggcacattctagaaggtggacctgatcatggagggtcaaatccacaaagcctggcaccctcttcgcccagttgatcatgtgaaccagctccctgtctgccaggttggtcagtaagcccatcatcgaagcttcactgaagggtctggtaggatcatactcggaatagagtatggggggctcagcatccaacaaggcactgaccatctggtcggccgtcagggacaaggccaggctgttcttcttagagcgtttgatcatgagcgggcttggccaaaggttggcagctctcatgtctccagcagatggctcgagatcgccatcttccagcaggcgcaccattgcccctgtttcactatccaggttacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatcctaaaatacacaaacaattagaatcagtagtttaacacattatacacttaaaaattttatatttaccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagtgactcccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacggacttggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcttgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacatggtggctacgtaataacttcgtatatggtttcttatacgaagttatgcggccgctttacgagggtaggaagtggtacggaaagttggtataagacaaaagtgttgtggaattgctccaggcgatctgacggttcactaaacgagctctgcttttataggcgcccaccgtacacgcctaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactcgcggccgcagagaaatgttctggcacctgcacttgcactggggacagcctattttgctagtttgttttgtttcgttttgttttgatggagagcgtatgttagtactatcgattcacacaaaaaaccaacacacagatgtaatgaaaataaagatattttattggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatgtaagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagcggatccgccgccaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctcttctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtgaaccggtcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttagagggcctatttcccatgattccttcatatttgcatatacgatacaaggctgttagagagataattagaattaatttgactgtaaacacaaagatattagtacaaaataataacttcgtataatgtatgctatacgaagttatcagacatgataagatacattgatgagtttggacaaaccacaactagaatgcagtgaaaaaaatgctttatttgtgaaatttgtgatgctattgctttatttgtaaccattataagctgcaataaacaaggtacctcaagcgccgggttttcgcgtcatgcaccacgtccgtgggccctcgggtacttcaacgtcagcagtaactgtaaatccgagccgttcatagaagggcaaattccttggcgctgacgtttcaagaaaggctggcactccggctcgttctgcggcttctactccgggcaataccaccgcggaaccaaggccctttccctgatgatcggggctaacgcccacagtagcgaggaaccaagctggttctttagggcggtgaggggcgaggagtccttccatttgttgctgagccgcgagacgagagccactaagctcagccattcggggaccaatttctgcaaatacagccccggcctcaacgctctccggagtcgtccacactgccactgcagccccgtcgtcggcgacccaaactttaccgatgtccaatcctaccctggtcaaaaaaagttcttgcaattctgtaacccgttcaatatgtctatcaggatcaactgtgtggcgtgtagcgggataatccgcgaaagcggcagccaatgttctcacggccctagggacgtcgtctcgagttgccagtctgacagtaggtttatattctgtcataggtccagggttctcctccacgtctccagcctgcttcagcaggctgaagttagtagctccggatcctttacctccatcaccagcgccaccagtagagtatctggccacagccacctcgtgctgctcgacgtaggtctcatcgtcggcctccttgattctttccagtctgtggtccacgttgtagacgccgggcatcttgaggttcgtagcgggtttcttggatctgtatgtggtctcaaggttgcagatcaggtggcccccgcccacgagcttcagggccatgtcacatgcgccttccaggccgccgtcagcggggtacatcgtctcggtggaggcctcccagccgagtgttttcttctgcatcacagggccgttggctgggaagttcacccctctaaccttgacgttgtagatgaggcagccgtcctggaggctggtgtcctgggtagcggtcagcacgcccccgtcttcgtatgtggtgactctctcccatgtgaagccctcagggaaggactgcttaaagaagtcggggatgcccggagggtgcttgatgaaggttctgctgccgtacatgaagctggtagccaggatgtcgaaggcgaaggggagagggccgccctcgacgaccttgattctcatggtctgggtgccctcgtagggcttgccttcgccctcggatgtgcacttgaagtggtggttgttcacggtgccctccatgtacagcttcatgggcatgttctccttaatcagctcgctcacggtggcggcgaattccgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgcctaccggtggatgtggaatgtgtgcgaggccagaggccacttgtgtagcgccaagtgcccagcggggctgctaaagcgcatgctccagactgccttgggaaaagcgctcccctacccataacttcgtataatgtatgctatacgaagttattttgcagttttaaaattatgttttaaaatggactatcatatgcttaccgtaacttgaaagtatttcgatttcttggctttatatatcttgtggaaaggacgaaacaccgggcactcttccgtgatctggtggataaattcgcaagggtatcatggcggacgaccgggattcgaaccccggatccggccgtccgccgtgatccatgcggttaccgcccgcgtgtcgaacccaggtgtgcgacgtcagacaacgggggagcgctcc

SEQ ID NO: 12 - DNA, 9344 bp, synthetic construct
  misc_feature 1..9344: note = source = /note="Description of Artificial Sequence: Synthetic polynucleotide"
  source 1..9344: mol_type = other DNA
gcctccacggccactagtccatagagcccaccgcatccccagcatgcctgctattgtcttcccaatcctcccccttgctgtcctgccccaccccaccccctagaatagaatgacacctactcagacaatgcgatgcaatttcctcattttattaggaaaggacagtgggagtggcaccttccagggtcaaggaaggcacgggggaggggcaaacaacagatggctggcaactagaaggcacagctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcatgagacgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatgcactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgtcagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtctctgtctcaaaaggaggtaggcgatccctactgtacggagtgcgccgagacaaccgagatcgtgttggtcgtagtgtcatgccaaatggaacgccggacgtagtcatggttgtggccatattatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcttaaaaatcaaaggggttctgccgcgcatcactatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcgcttgtcgtcatcgtctttgtagtcgggaggcggcggcgacggcgacggggacgacacgtcctccatggttggtggacgtcgcgccgcaccgcgtccgcgctcgggggtggtttcgcgctgctcctcttcccgactggccatggtggccgaggataacttcgtatatggtttcttatacgaagttatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatctgctacgtatcaagctgtggcagggaaaccctctgcctcccccgtgatgtaatacttttgcaaggaatgcgatgaagtagagcccgcagtggccaagtggctttggtccgtctcctccacggatgcccctccacggctagtgggcgcatgtaggcggtgggcgtccgccgcctccagcagcaggtcatagaggggcaccacgttcttgcacttcatgctgtacagatgctccatgcctttgttactcatgtgtcggatgtgggagaggatgaggaggagctgggccagccgctggtgctgctgctgcagggtcaggcctgccttggccatcaggtggatcaaagtgtctgtgatcttgtccaggactcggtggatatggtccttctcttccagagacttcagggtgctggacagaaatgtgtacactccagaattaagcaaaataatagatttgaggcacacaaactcctctccctgcagattcatcatgcggaaccgagatgatgtagccagcagcatgtcgaagatctccaccatgccctctacacattttccctggttcctgtccaagagcaagttaggagcaaacagtagcttcactgggtgctccatggagcgccagacgagaccaatcatcaggatctctagccaggcacattctagaaggtggacctgatcatggagggtcaaatccacaaagcctggcaccctcttcgcccagttgatcatgtgaaccagctccctgtctgccaggttggtcagtaagcccatcatcgaagcttcactgaagggtctggtaggatcatactcggaatagagtatggggggctcagcatccaacaaggcactgaccatctggtcggccgtcagggacaaggccaggctgttcttcttagagcgtttgatcatgagcgggcttggccaaaggttggcagctctcatgtctccagcagatggctcgagatcgccatcttccagcaggcgcaccattgcccctgtttcactatccaggttacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatcctaaaatacacaaacaattagaatcagtagtttaacacattatacacttaaaaattttatatttaccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagtgactcccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacggacttggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcttgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacatggtggctacgtaataacttcgtatatggtttcttatacgaagttatgcggccgctttacgagggtaggaagtggtacggaaagttggtataagacaaaagtgttgtggaattgctccaggcgatctgacggttcactaaacgagctctgcttttataggcgcccaccgtacacgcctaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactcgcggccgcagagaaatgttctggcacctgcacttgcactggggacagcctattttgctagtttgttttgtttcgttttgttttgatggagagcgtatgttagtactatcgattcacacaaaaaaccaacacacagatgtaatgaaaataaagatattttattggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatgtaagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagcggatccgccgccaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctcttctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggggatccggagctactaacttcagcctgctgaagcaggctggagacgtggaggagaaccctggacctatgacagaatataaacctactgtcagactggcaactcgagacgacgtccctagggccgtgagaacattggctgccgctttcgcggattatcccgctacacgccacacagttgatcctgatagacatattgaacgggttacagaattgcaagaactttttttgaccagggtaggattggacatcggtaaagtttgggtcgccgacgacggggctgcagtggcagtgtggacgactccggagagcgttgaggccggggctgtatttgcagaaattggtccccgaatggctgagcttagtggctctcgtctcgcggctcagcaacaaatggaaggactcctcgcccctcaccgccctaaagaaccagcttggttcctcgctactgtgggcgttagccccgatcatcagggaaagggccttggttccgcggtggtattgcccggagtagaagccgcagaacgagccggagtgccagcctttcttgaaacgtcagcgccaaggaatttgcccttctatgaacggctcggatttacagttactgctgacgttgaagtacccgagggcccacggacgtggtgcatgacgcgaaaacccggcgcttgagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttagtttaaacgggcccttaattaatcgatgtaggatgttgcccctcctgacgcggtaggagaaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggcccgagactcgaaccgggggtcctgcgactcaacccttggaaaataaccctccggctacagggagcgagccacttaatgctttcgctttccagcctaaccgcttacgccgcgcgcggccagtggccaaaaaagctagcgcagcagccgccgcgcctggaaggaagccaaaaggagcgctcccccgttgtctgacgtcgcacacctgggttcgacacgcgggcggtaaccgcatggatcacggcggacggccggatccggggttcgaaccccggtcgtccgccatgatacccttgcgaatttatccaccagaccacggaagagtgcccgcttacaggctctccttttgcacggtctagagcgtcaacgactgcgcacgcctcaccggccagagcgtcccgaccatggagcactttttgccgctgcgcaacatctggaaccgcgtccgcgactttccgcgcgcctccaccaccgccgccggcatcacctggatgtccaggtacatctacggattacggggcccattggtatg

SEQ ID NO: 13 - DNA, 161 bp, synthetic construct
  misc_feature 1..161: note = source = /note="Description of Artificial Sequence: Synthetic polynucleotide"
  source 1..161: mol_type = other DNA
gggcactcttccgtggtctggtggataaattcgcaagggtatcatggcggacgaccggggttcgaaccccggatccggccgtccgccgtgatccatgcggttaccgcccgcgtgtcgaacccaggtgtgcgacgtcagacaacgggggagcgctccttttt

SEQ ID NO: 14 - DNA, 151 bp, synthetic construct
  misc_feature 1..151: note = source = /note="Description of Artificial Sequence: Synthetic polynucleotide"
  source 1..151: mol_type = other DNA
gggcactcttccgtggtctggtggataaattcgcaagggtatcatggcggacgaccggccggatccggccgtccgccgtgatccatgcggttaccgcccgcgtgtcgaacccaggtgtgcgacgtcagacaacgggggagcgctccttttt

SEQ ID NO: 15 - DNA, 161 bp, synthetic construct
  misc_feature 1..161: note = source = /note="Description of Artificial Sequence: Synthetic polynucleotide"
  source 1..161: mol_type = other DNA
gggcactcttccgtgatctggtggataaattcgcaagggtatcatggcggacgaccggggttcgaaccccggatccggccgtccgccgtgatccatgcggttaccgcccgcgtgtcgaacccaggtgtgcgacgtcagacaacgggggagcgctccttttt

SEQ ID NO: 16 - DNA, 161 bp, synthetic construct
  misc_feature 1..161: note = source = /note="Description of Artificial Sequence: Synthetic polynucleotide"
  source 1..161: mol_type = other DNA
gggcactcttccgtgatctggtggataaattcgcaagggtatcatggcggacgaccgggattcgaaccccggatccggccgtccgccgtgatccatgcggttaccgcccgcgtgtcgaacccaggtgtgcgacgtcagacaacgggggagcgctccttttt

SEQ ID NO: 17 - DNA, 393 bp, synthetic construct
  misc_feature 1..393: note = source = /note="Description of Artificial Sequence: Synthetic polynucleotide"
  source 1..393: mol_type = other DNA
cgatggagggcctatttcccatgattccttcatatttgcatatacgatacaaggctgttagagagataattagaattaatttgactgtaaacacaaagatattagtacaaaataataacttcgtataatgtatgctatacgaagttattttgcagttttaaaattatgttttaaaatggactatcatatgcttaccgtaacttgaaagtatttcgatttcttggctttatatatcttgtggaaaggacgaaacaccgggcactcttccgtggtctggtggataaattcgcaagggtatcatggcggacgaccggccggatccggccgtccgccgtgatccatgcggttaccgcccgcgtgtcgaacccaggtgtgcgacgtcagacaacgggg

SEQ ID NO: 18 - DNA, 417 bp, synthetic construct
  misc_feature 1..417: note = source = /note="Description of Artificial Sequence: Synthetic polynucleotide"
  source 1..417: mol_type = other DNA
cgatggagggcctatttcccatgattccttcatatttgcatatacgatacaaggctgttagagagataattagaattaatttgactgtaaacacaaagatattagtacaaaataataacttcgtataatgtatgctatacgaagttattttgcagttttaaaattatgttttaaaatggactatcatatgcttaccgtaacttgaaagtatttcgatttcttggctttatatatcttgtggaaaggacgaaacaccgggcactcttccgtgatctggtggataaattcgcaagggtatcatggcggacgaccggggttcgaaccccggatccggccgtccgccgtgatccatgcggttaccgcccgcgtgtcgaacccaggtgtgcgacgtcagacaacgggggagcgctccttttt

SEQ ID NO: 19 - DNA, 428 bp, synthetic construct
  misc_feature 1..428: note = source = /note="Description of Artificial Sequence: Synthetic polynucleotide"
  source 1..428: mol_type = other DNA
ttcactagaatcgatggagggcctatttcccatgattccttcatatttgcatatacgatacaaggctgttagagagataattagaattaatttgactgtaaacacaaagatattagtacaaaataataacttcgtataatgtatgctatacgaagttattttgcagttttaaaattatgttttaaaatggactatcatatgcttaccgtaacttgaaagtatttcgatttcttggctttatatatcttgtggaaaggacgaaacaccgggcactcttccgtgatctggtggataaattcgcaagggtatcatggcggacgaccgggattcgaaccccggatccggccgtccgccgtgatccatgcggttaccgcccgcgtgtcgaacccaggtgtgcgacgtcagacaacgggggagcgctccttttt

SEQ ID NO: 20 - DNA, 544 bp, synthetic construct
  misc_feature 1..544: note = source = /note="Description of Artificial Sequence: Synthetic polynucleotide"
  source 1..544: mol_type = other DNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatgtaagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 21 - DNA, 744 bp, synthetic construct
  misc_feature 1..744: note = source = /note="Description of Artificial Sequence: Synthetic polynucleotide"
  source 1..744: mol_type = other DNA
atgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctcttctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccggg

SEQ ID NO: 22 - DNA, 379 bp, synthetic construct
  misc_feature 1..379: note = source = /note="Description of Artificial Sequence: Synthetic polynucleotide"
  source 1..379: mol_type = other DNA
gagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaa

SEQ ID NO: 23 - DNA, 11051 bp, synthetic construct
  misc_feature 1..11051: note = source = /note="Description of Artificial Sequence: Synthetic polynucleotide"
  source 1..11051: mol_type = other DNA
ggtacccaactccatgcttaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactaggagacactttcaataaaggcaaatgtttttatttgtacactctcgggtgattatttaccccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcggggaggcggcggcgacggcgacggggacgacacgtcctccatggttggtggacgtcgcgccgcaccgcgtccgcgctcgggggtggtttcgcgctgctcctcttcccgactggccatttccttctcctataggcagaaaaagatcatggagtcagtcgagaaggaggacagcctaaccgccccctttgagttcgccaccaccgcctccaccgatgccgccaacgcgcctaccaccttccccgtcgaggcacccccgcttgaggaggaggaagtgattatcgagcaggacccaggttttgtaagcgaagacgacgaggatcgctcagtaccaacagaggataaaaagcaagaccaggacgacgcagaggcaaacgaggaacaagtcgggcggggggaccaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctgttctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcgacgaagtgccaaaaatctttgagggtcttggacgcgacgagaaacgcgcggcaaacgctctgcaacaagaaaacagcgaaaatgaaagtcactgtggagtgctggtggaacttgagggtgacaacgcgcgcctagccgtgctgaaacgcagcatcgaggtcacccactttgcctacccggcacttaacctaccccccaaggttatgagcacagtcatgagcgagctgatcgtgcgccgtgcacgacccctggagagggatgcaaacttgcaagaacaaaccgaggagggcctacccgcagttggcgatgagcagctggcgcgctggcttgagacgcgcgagcctgccgacttggaggagcgacgcaagctaatgatggccgcagtgcttgttaccgtggagcttgagtgcatgcagcggttctttgctgacccggagatgcagcgcaagctagaggaaacgttgcactacacctttcgccagggctacgtgcgccaggcctgcaaaatttccaacgtggagctctgcaacctggtctcctaccttggaattttgcacgaaaaccgcctcgggcaaaacgtgcttcattccacgctcaagggcgaggcgcgccgcgactacgtccgcgactgcgtttacttatttctgtgctacacctggcaaacggccatgggcgtgtggcagcaatgcctggaggagcgcaacctaaaggagctgcagaagctgctaaagcaaaacttgaaggacctatggacggccttcaacgagcgctccgtggccgcgcacctggcggacattatcttccccgaacgcctgcttaaaaccctgcaacagggtctgccagacttcaccagtcaaagcatgttgcaaaactttaggaactttatcctagagcgttcaggaattctgcccgccacctgctgtgcgcttcctagcgactttgtgcccattaagtaccgtgaatgccctccgccgctttggggtcactgctaccttctgcagctagccaactaccttgcctaccactccgacatcatggaagacgtgagcggtgacggcctactggagtgtcactgtcgctgcaacctatgcaccccgcaccgctccctggtctgcaattcgcaactgcttagcgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacatccttggccaattgcaagccatcaacaaagcccgccaagagtttctgctacgaaagggacggggggtttacctggacccccagtccggcgaggagctcaacccaatccccccgccgccgcagccctatcagcagccgcgggcccttgcttcccaggatggcacccaaaaagaagctgcagctgccgccgccgccacccacggacgaggaggaatactgggacagtcaggcagaggaggttttggacgaggaggaggagatgatggaagactgggacagcctagacgaagcttccgaggccgaagaggtgtcagacgaaacaccgtcaccctcggtcgcattcccctcgccggcgccccagaaattggcaaccgttcccagcatcgctacaacctccgctcctcaggcgccgccggcactgcctgttcgccgacccaaccgtagatgggacaccactggaaccagggccggtaagtctaagcagccgccgccgttagcccaagagcaacaacagcgccaaggctaccgctcgtggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccctactgcaccggcggcagcggcagcggcagcaacagcagcggtcacacagaagcaaaggcgaccggatagcaagactctgacaaagcccaagaaatccacagcggcggcagcagcaggaggaggagcgctgcgtctggcgcccaacgaacccgtatcgacccgcgagcttagaaataggatttttcccactctgtatgctatatttcaacaaagcaggggccaagaacaagagctgaaaataaaaaacaggtctctgcgctccctcacccgcagctgcctgtatcacaaaagcgaagatcagcttcggcgcacgctggaagacgcggaggctctcttcagcaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcctcgaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcgttttagggcggagtaacttgcatgtattgggaattgtagtttttttaaaatgggaagtgacgtatcgtgggaaaacggaagtgaagatttgaggaagttgtgggttttttggctttcgtttctgggcgtaggttcgcgtgcggttttctgggtgttttttgtggactttaaccgttacgtcattttttagtcctatatatactcgctctgtacttggccctttttacactgtgactgattgagctggtgccgtgtcgagtggtgttttttaataggtttttttactggtaaggctgactgttatggctgccgctgtggaagcgctgtatgttgttctggagcgggagggtgctattttgcctaggcaggagggtttttcaggtgtttatgtgtttttctctcctattaattttgttatacctcctatgggggctgtaatgttgtctctacgcctgcgggtatgtattcccccgggctatttcggtcgctttttagcactgaccgatgttaaccaacctgatgtgtttaccgagtcttacattatgactccggacatgaccgaggaactgtcggtggtgctttttaatcacggtgaccagtttttttacggtcacgccggcatggccgtagtccgtcttatgcttataagggttgtttttcctgttgtaagacaggcttctaatgtttaaatgtttttttttttgttattttattttgtgtttaatgcaggaacccgcagacatgtttgagagaaaaatggtgtctttttctgtggtggttccggaacttacctgcctttatctgcatgagcatgactacgatgtgcttgcttttttgcgcgaggctttgcctgattttttgagcagcaccttgcattttatatcgccgcccatgcaacaagcttacataggggctacgctggttagcatagctccgagtatgcgtgtcataatcagtgtgggttcttttgtcatggttcctggcggggaagtggccgcgctggtccgtgcagacctgcacgattatgttcagctggccctgcgaagggacctacgggatcgcggtatttttgttaatgttccgcttttgaatcttatacaggtctgtgaggaacctgaatttttgcaatcatgattcgctgcttgaggctgaaggtggagggcgctctggagcagatttttacaatggccggacttaatattcgggatttgcttagagacatattgataaggtggcgagatgaaaattatttgggcatggttgaaggtgctggaatgtttatagaggagattcaccctgaagggtttagcctttacgtccacttggacgtgagggcagtttgccttttggaagccattgtgcaacatcttacaaatgccattatctgttctttggctgtagagtttgaccacgccaccggaggggagcgcgttcacttaatagatcttcattttgaggttttggataatcttttggaataaaaaaaaaaaaacatggttcttccagctcttcccgctcctcccgtgtgtgactcgcagaacgaatgtgtaggttggctgggtgtggcttattctgcggtggtggatgttatcagggcagcggcgcatgaaggagtttacatagaacccgaagccagggggcgcctggatgctttgagagagtggatatactacaactactacacagagcgagctaagcgacgagaccggagacgcagatctgtttgtcacgcccgcacctggttttgcttcaggaaatatgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttccccttcgccgcccgttgagcaaccgcaagttggacagcagcctgtggctcagcagctggacagcgacatgaacttaagcgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatcaatataagctatgtggtggtggggctatactactgaatgaaaaatgacttgaaattttctgcaattgaaaaataaacacgttgaaacataacatgcaacaggttcacgattctttattcctgggcaatgtaggagaaggtgtaagagttggtagcaaaagtttcagtggtgtattttccactttcccaggaccatgtaaaagacatagagtaagtgcttacctcgctagtttctgtggattcactagaatcgatgtcgacgggcactcttccgtggtctggtggataaattcgcaagggtatcatggcggacgaaccggtaccgaaccccggatccggccgtccgccgtgatccatgcggttaccgcccgcgtgtcgaacccaggtgtgcgacgtcagacaacgggggagcgctcctttttcatatgatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgatggatcc

SEQ ID NO: 24 - DNA, 11051 bp, synthetic construct
  misc_feature 1..11051: note = source = /note="Description of Artificial Sequence: Synthetic polynucleotide"
  source 1..11051: mol_type = other DNA
ggtacccaactccatgcttaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactaggagacactttcaataaaggcaaatgtttttatttgtacactctcgggtgattatttaccccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcggggaggcggcggcgacggcgacggggacgacacgtcctccatggttggtggacgtcgcgccgcaccgcgtccgcgctcgggggtggtttcgcgctgctcctcttcccgactggccatttccttctcctataggcagaaaaagatcatggagtcagtcgagaaggaggacagcctaaccgccccctttgagttcgccaccaccgcctccaccgatgccgccaacgcgcctaccaccttccccgtcgaggcacccccgcttgaggaggaggaagtgattatcgagcaggacccaggttttgtaagcgaagacgacgaggatcgctcagtaccaacagaggataaaaagcaagaccaggacgacgcagaggcaaacgaggaacaagtcgggcggggggaccaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctgttctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcgacgaagtgccaaaaatctttgagggtcttggacgcgacgagaaacgcgcggcaaacgctctgcaacaagaaaacagcgaaaatgaaagtcactgtggagtgctggtggaacttgagggtgacaacgcgcgcctagccgtgctgaaacgcagcatcgaggtcacccactttgcctacccggcacttaacctaccccccaaggttatgagcacagtcatgagcgagctgatcgtgcgccgtgcacgacccctggagagggatgcaaacttgcaagaacaaaccgaggagggcctacccgcagttggcgatgagcagctggcgcgctggcttgagacgcgcgagcctgccgacttggaggagcgacgcaagctaatgatggccgcagtgcttgttaccgtggagcttgagtgcatgcagcggttctttgctgacccggagatgcagcgcaagctagaggaaacgttgcactacacctttcgccagggctacgtgcgccaggcctgcaaaatttccaacgtggagctctgcaacctggtctcctaccttggaattttgcacgaaaaccgcctcgggcaaaacgtgcttcattccacgctcaagggcgaggcgcgccgcgactacgtccgcgactgcgtttacttatttctgtgctacacctggcaaacggccatgggcgtgtggcagcaatgcctggaggagcgcaacctaaaggagctgcagaagctgctaaagcaaaacttgaaggacctatggacggccttcaacgagcgctccgtggccgcgcacctggcggacattatcttccccgaacgcctgcttaaaaccctgcaacagggtctgccagacttcaccagtcaaagcatgttgcaaaactttaggaactttatcctagagcgttcaggaattctgcccgccacctgctgtgcgcttcctagcgactttgtgcccattaagtaccgtgaatgccctccgccgctttggggtcactgctaccttctgcagctagccaactaccttgcctaccactccgacatcatggaagacgtgagcggtgacggcctactggagtgtcactgtcgctgcaacctatgcaccccgcaccgctccctggtctgcaattcgcaactgcttagcgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacatccttggccaattgcaagccatcaacaaagcccgccaagagtttctgctacgaaagggacggggggtttacctggacccccagtccggcgaggagctcaacccaatccccccgccgccgcagccctatcagcagccgcgggcccttgcttcccaggatggcacccaaaaagaagctgcagctgccgccgccgccacccacggacgaggaggaatactgggacagtcaggcagaggaggttttggacgaggaggaggagatgatggaagactgggacagcctagacgaagcttccgaggccgaagaggtgtcagacgaaacaccgtcaccctcggtcgcattcccctcgccggcgccccagaaattggcaaccgttcccagcatcgctacaacctccgctcctcaggcgccgccggcactgcctgttcgccgacccaaccgtagatgggacaccactggaaccagggccggtaagtctaagcagccgccgccgttagcccaagagcaacaacagcgccaaggctaccgctcgtggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccctactgcaccggcggcagcggcagcggcagcaacagcagcggtcacacagaagcaaaggcgaccggatagcaagactctgacaaagcccaagaaatccacagcggcggcagcagcaggaggaggagcgctgcgtctggcgcccaacgaacccgtatcgacccgcgagcttagaaataggatttttcccactctgtatgctatatttcaacaaagcaggggccaagaacaagagctgaaaataaaaaacaggtctctgcgctccctcacccgcagctgcctgtatcacaaaagcgaagatcagcttcggcgcacgctggaagacgcggaggctctcttcagcaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcctcgaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcgttttagggcggagtaacttgcatgtattgggaattgtagtttttttaaaatgggaagtgacgtatcgtgggaaaacggaagtgaagatttgaggaagttgtgggttttttggctttcgtttctgggcgtaggttcgcgtgcggttttctgggtgttttttgtggactttaaccgttacgtcattttttagtcctatatatactcgctctgtacttggccctttttacactgtgactgattgagctggtgccgtgtcgagtggtgttttttaataggtttttttactggtaaggctgactgttatggctgccgctgtggaagcgctgtatgttgttctggagcgggagggtgctattttgcctaggcaggagggtttttcaggtgtttatgtgtttttctctcctattaattttgttatacctcctatgggggctgtaatgttgtctctacgcctgcgggtatgtattcccccgggctatttcggtcgctttttagcactgaccgatgttaaccaacctgatgtgtttaccgagtcttacattatgactccggacatgaccgaggaactgtcggtggtgctttttaatcacggtgaccagtttttttacggtcacgccggcatggccgtagtccgtcttatgcttataagggttgtttttcctgttgtaagacaggcttctaatgtttaaatgtttttttttttgttattttattttgtgtttaatgcaggaacccgcagacatgtttgagagaaaaatggtgtctttttctgtggtggttccggaacttacctgcctttatctgcatgagcatgactacgatgtgcttgcttttttgcgcgaggctttgcctgattttttgagcagcaccttgcattttatatcgccgcccatgcaacaagcttacataggggctacgctggttagcatagctccgagtatgcgtgtcataatcagtgtgggttcttttgtcatggttcctggcggggaagtggccgcgctggtccgtgcagacctgcacgattatgttcagctggccctgcgaagggacctacgggatcgcggtatttttgttaatgttccgcttttgaatcttatacaggtctgtgaggaacctgaatttttgcaatcatgattcgctgcttgaggctgaaggtggagggcgctctggagcagatttttacaatggccggacttaatattcgggatttgcttagagacatattgataaggtggcgagatgaaaattatttgggcatggttgaaggtgctggaatgtttatagaggagattcaccctgaagggtttagcctttacgtccacttggacgtgagggcagtttgccttttggaagccattgtgcaacatcttacaaatgccattatctgttctttggctgtagagtttgaccacgccaccggaggggagcgcgttcacttaatagatcttcattttgaggttttggataatcttttggaataaaaaaaaaaaaacatggttcttccagctcttcccgctcctcccgtgtgtgactcgcagaacgaatgtgtaggttggctgggtgtggcttattctgcggtggtggatgttatcagggcagcggcgcatgaaggagtttacatagaacccgaagccagggggcgcctggatgctttgagagagtggatatactacaactactacacagagcgagctaagcgacgagaccggagacgcagatctgtttgtcacgcccgcacctggttttgcttcaggaaatatgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttccccttcgccgcccgttgagcaaccgcaagttggacagcagcctgtggctcagcagctggacagcgacatgaacttaagcgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatcaatataagctatgtggtggtggggctatactactgaatgaaaaatgacttgaaattttctgcaattgaaaaataaacacgttgaaacataacatgcaacaggttcacgattctttattcctgggcaatgtaggagaaggtgtaagagttggtagcaaaagtttcagtggtgtattttccactttcccaggaccatgtaaaagacatagagtaagtgcttacctcgctagtttctgtggattcactagaatcgatgtcgacgggcactcttccgtggtctggtggataaattcgcaagggtatcatggcggacgaccgggattcgaaccccggatccggccgtccgccgtgatccatgcggttaccgcccgcgtgtcgaacccaggtgtgcgacgtcagacaacgggggagcgctcctttttcatatgatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgatggatcc

SEQ ID NO: 25 - DNA, 6770 bp, synthetic construct
  misc_feature 1..6770: note = source = /note="Description of Artificial Sequence: Synthetic polynucleotide"
  source 1..6770: mol_type = other DNA
ccgcggccgccaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatggccagtcgggaagaggagcagcgcgaaaccacccccgagcgcggacgcggtgcggcgcgacgtccaccaaccatggaggacgtgtcgtccccgtcgccgtcgccgccgcctccccgcgcgcccccaaaaaagcggctgaggcggcgtctcgagtccgaggacgaagaagactcgtcacaagatgcgctggtgccgcgcacacccagcccgcggccatcgacctcgacggcggatttggccattgcgtccaaaaagaaaaagaagcgcccctctcccaagcccgagcgcccgccatccccagaggtgatcgtggacagcgaggaagaaagagaagatgtggcgctacaaatggtgggtttcagcaacccaccggtgctaatcaagcacggcaagggaggtaagcgcacggtgcggcggctgaatgaagacgacccagtggcgcggggtatgcggacgcaagaggaaaaggaagagtccagtgaagcggaaagtgaaagcacggtgataaacccgctgagcctgccgatcgtgtctgcgtgggagaagggcatggaggctgcgcgcgcgttgatggacaagtaccacgtggataacgatctaaaggcaaacttcaagctactgcctgaccaagtggaagctctggcggccgtatgcaagacctggctaaacgaggagcaccgcgggttgcagctgaccttcaccagcaacaagacctttgtgacgatgatggggcgattcctgcaggcgtacctgcagtcgtttgcagaggtaacctacaagcaccacgagcccacgggctgcgcgttgtggctgcaccgctgcgctgagatcgaaggcgagcttaagtgtctacacgggagcattatgataaataaggagcacgtgattgaaatggatgtgacgagcgaaaacgggcagcgcgcgctgaaggagcagtctagcaaggccaagatcgtgaagaaccggtggggccgaaatgtggtgcagatctccaacaccgacgcaaggtgctgcgtgcatgacgcggcctgtccggccaatcagttttccggcaagtcttgcggcatgttcttctctgaaggcgcaaaggctcaggtggcttttaagcagatcaaggctttcatgcaggcgctgtatcctaacgcccagaccgggcacggtcaccttctgatgccactacggtgcgagtgcaactcaaagcctgggcatgcaccctttttgggaaggcagctaccaaagttgactccgttcgccctgagcaacgcggaggacctggacgcggatctgatctccgacaagagcgtgctggccagcgtgcaccacccggcgctgatagtgttccagtgctgcaaccctgtgtatcgcaactcgcgcgcgcagggcggaggccccaactgcgacttcaagatatcggcgcccgacctgctaaacgcgttggtgatggtgcgcagcctgtggagtgaaaacttcaccgagctgccgcggatggttgtgcctgagtttaagtggagcactaaacaccagtatcgcaacgtgtccctgccagtggcgcatagcgatgcgcggcagaacccctttgatttttaaacccagctttcttgtacaaagtgggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtagcaactttattatacatagttgatggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagcggccgcggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctctcttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcggcgcgcctcgatgtaggatgttgcccctcctgacgcggtaggagaaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggcccgagactcgaaccgggggtcctgcgactcaacccttggaaaataaccctccggctacagggagcgagccacttaatgctttcgctttccagcctaaccgcttacgccgcgcgcggccagtggccaaaaaagctagcgcagcagccgccgcgcctggaaggaagccaaaaggagcgctcccccgttgtctgacgtcgcacacctgggttcgacacgcgggcggtaaccgcatggatcacggcggacggccggatccggggttcgaaccccggtcgtccgccatgatacccttgcgaatttatccaccagaccacggaagagtgcccgcttacaggctctccttttgcacggtctagagcgtcaacgactgcgcacgcctcaccggccagagcgtcccgaccatggagcactttttgccgctgcgcaacatctggaaccgcgtccgcgactttccgcgcgcctccaccaccgccgccggcatcacctggatgtccaggtacatctacggattacgggcgcg

SEQ ID NO: 26 - DNA, 6770 bp, synthetic construct
  misc_feature 1..6770: note = source = /note="Description of Artificial Sequence: Synthetic polynucleotide"
  source 1..6770: mol_type = other DNA
ccgcggccgccaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtagacccagctttcttgtacaaagtgggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggccagtcgggaagaggagcagcgcgaaaccacccccgagcgcggacgcggtgcggcgcgacgtccaccaaccatggaggacgtgtcgtccccgtcgccgtcgccgccgcctccccgcgcgcccccaaaaaagcggctgaggcggcgtctcgagtccgaggacgaagaagactcgtcacaagatgcgctggtgccgcgcacacccagcccgcggccatcgacctcgacggcggatttggccattgcgtccaaaaagaaaaagaagcgcccctctcccaagcccgagcgcccgccatccccagaggtgatcgtggacagcgaggaagaaagagaagatgtggcgctacaaatggtgggtttcagcaacccaccggtgctaatcaagcacggcaagggaggtaagcgcacggtgcggcggctgaatgaagacgacccagtggcgcggggtatgcggacgcaagaggaaaaggaagagtccagtgaagcggaaagtgaaagcacggtgataaacccgctgagcctgccgatcgtgtctgcgtgggagaagggcatggaggctgcgcgcgcgttgatggacaagtaccacgtggataacgatctaaaggcaaacttcaagctactgcctgaccaagtggaagctctggcggccgtatgcaagacctggctaaacgaggagcaccgcgggttgcagctgaccttcaccagcaacaagacctttgtgacgatgatggggcgattcctgcaggcgtacctgcagtcgtttgcagaggtaacctacaagcaccacgagcccacgggctgcgcgttgtggctgcaccgctgcgctgagatcgaaggcgagcttaagtgtctacacgggagcattatgataaataaggagcacgtgattgaaatggatgtgacgagcgaaaacgggcagcgcgcgctgaaggagcagtctagcaaggccaagatcgtgaagaaccggtggggccgaaatgtggtgcagatctccaacaccgacgcaaggtgctgcgtgcatgacgcggcctgtccggccaatcagttttccggcaagtcttgcggcatgttcttctctgaaggcgcaaaggctcaggtggcttttaagcagatcaaggctttcatgcaggcgctgtatcctaacgcccagaccgggcacggtcaccttctgatgccactacggtgcgagtgcaactcaaagcctgggcatgcaccctttttgggaaggcagctaccaaagttgactccgttcgccctgagcaacgcggaggacctggacgcggatctgatctccgacaagagcgtgctggccagcgtgcaccacccggcgctgatagtgttccagtgctgcaaccctgtgtatcgcaactcgcgcgcgcagggcggaggccccaactgcgacttcaagatatcggcgcccgacctgctaaacgcgttggtgatggtgcgcagcctgtggagtgaaaacttcaccgagctgccgcggatggttgtgcctgagtttaagtggagcactaaacaccagtatcgcaacgtgtccctgccagtggcgcatagcgatgcgcggcagaacccctttgatttttaacaactttattatacatagttgatggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagcggccgcggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctctcttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcggcgcgcctcgatgtaggatgttgcccctcctgacgcggtaggagaaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggcccgagactcgaaccgggggtcctgcgactcaacccttggaaaataaccctccggctacagggagcgagccacttaatgctttcgctttccagcctaaccgcttacgccgcgcgcggccagtggccaaaaaagctagcgcagcagccgccgcgcctggaaggaagccaaaaggagcgctcccccgttgtctgacgtcgcacacctgggttcgacacgcgggcggtaaccgcatggatcacggcggacggccggatccggggttcgaaccccggtcgtccgccatgatacccttgcgaatttatccaccagaccacggaagagtgcccgcttacaggctctccttttgcacggtctagagcgtcaacgactgcgcacgcctcaccggccagagcgtcccgaccatggagcactttttgccgctgcgcaacatctggaaccgcgtccgcgactttccgcgcgcctccaccaccgccgccggcatcacctggatgtccaggtacatctacggattacgggcgcg

SEQ ID NO: 27 - DNA, 14227 bp, synthetic construct
  misc_feature 1..14227: note = source = /note="Description of Artificial Sequence: Synthetic polynucleotide"
  source 1..14227: mol_type = other DNA
tggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccactagtccatagagcccaccgcatccccagcatgcctgctattgtcttcccaatcctcccccttgctgtcctgccccaccccaccccctagaatagaatgacacctactcagacaatgcgatgcaatttcctcattttattaggaaaggacagtgggagtggcaccttccagggtcaaggaaggcacgggggaggggcaaacaacagatggctggcaactagaaggcacagctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcatgagacgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatgcactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgtcagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtctctgtctcaaaaggaggtaggcgatccctactgtacggagtgcgccgagacaaccgagatcgtgttggtcgtagtgtcatgccaaatggaacgccggacgtagtcatggttgtggccatattatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcttaaaaatcaaaggggttctgccgcgcatcactatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcgcttgtcgtcatcgtctttgtagtcgggaggcggcggcgacggcgacggggacgacacgtcctccatggttggtggacgtcgcgccgcaccgcgtccgcgctcgggggtggtttcgcgctgctcctcttcccgactggccatggtggccgaggataacttcgtatatggtttcttatacgaagttatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatctgctacgtatcaagctgtggcagggaaaccctctgcctcccccgtgatgtaatacttttgcaaggaatgcgatgaagtagagcccgcagtggccaagtggctttggtccgtctcctccacggatgcccctccacggctagtgggcgcatgtaggcggtgggcgtccgccgcctccagcagcaggtcatagaggggcaccacgttcttgcacttcatgctgtacagatgctccatgcctttgttactcatgtgtcggatgtgggagaggatgaggaggagctgggccagccgctggtgctgctgctgcagggtcaggcctgccttggccatcaggtggatcaaagtgtctgtgatcttgtccaggactcggtggatatggtccttctcttccagagacttcagggtgctggacagaaatgtgtacactccagaattaagcaaaataatagatttgaggcacacaaactcctctccctgcagattcatcatgcggaaccgagatgatgtagccagcagcatgtcgaagatctccaccatgccctctacacattttccctggttcctgtccaagagcaagttaggagcaaacagtagcttcactgggtgctccatggagcgccagacgagaccaatcatcaggatctctagccaggcacattctagaaggtggacctgatcatggagggtcaaatccacaaagcctggcaccctcttcgcccagttgatcatgtgaaccagctccctgtctgccaggttggtcagtaagcccatcatcgaagcttcactgaagggtctggtaggatcatactcggaatagagtatggggggctcagcatccaacaaggcactgaccatctggtcggccgtcagggacaaggccaggctgttcttcttagagcgtttgatcatgagcgggcttggccaaaggttggcagctctcatgtctccagcagatggctcgagatcgccatcttccagcaggcgcaccattgcccctgtttcactatccaggttacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatcctaaaatacacaaacaattagaatcagtagtttaacacattatacacttaaaaattttatatttaccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagtgactcccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacggacttggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcttgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacatggtggctacgtaataacttcgtatatggtttcttatacgaagttatgcggccgctttacgagggtaggaagtggtacggaaagttggtataagacaaaagtgttgtggaattgctccaggcgatctgacggttcactaaacgagctctgcttttataggcgcccaccgtacacgcctaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactcgcggccgcagagaaatgttctggcacctgcacttgcactggggacagcctattttgctagtttgttttgtttcgttttgttttgatggagagcgtatgttagtactatcgattcacacaaaaaaccaacacacagatgtaatgaaaataaagatattttattggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatgtaagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagcggatccgccgccaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctcttctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtgaaccggtcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttagagggcctatttcccatgattccttcatatttgcatatacgatacaaggctgttagagagataattagaattaatttgactgtaaacacaaagatattagtacaaaataataacttcgtataatgtatgctatacgaagttatcagacatgataagatacattgatgagtttggacaaaccacaactagaatgcagtgaaaaaaatgctttatttgtgaaatttgtgatgctattgctttatttgtaaccattataagctgcaataaacaaggtacctcaagcgccgggttttcgcgtcatgcaccacgtccgtggtagaactagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaagagaattcgccgccaccatgacagaatataaacctactgtcagactggcaactcgagacgacgtccctagggccgtgagaacattggctgccgctttcgcggattatcccgctacacgccacacagttgatcctgatagacatattgaacgggttacagaattgcaagaactttttttgaccagggtaggattggacatcggtaaagtttgggtcgccgacgacggggctgcagtggcagtgtggacgactccggagagcgttgaggccggggctgtatttgcagaaattggtccccgaatggctgagcttagtggctctcgtctcgcggctcagcaacaaatggaaggactcctcgcccctcaccgccctaaagaaccagcttggttcctcgctactgtgggcgttagccccgatcatcagggaaagggccttggttccgcggtggtattgcccggagtagaagccgcagaacgagccggagtgccagcctttcttgaaacgtcagcgccaaggaatttgcccttctatgaacggctcggatttacagttactgctgacgttgaagtacccgagggcccacggacgtggtgcatgacgcgaaaacccggcgcttgaaccggtcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttaggttgggaaaagcgctcccctacccataacttcgtataatgtatgctatacgaagttattttgcagttttaaaattatgttttaaaatggactatcatatgcttaccgtaacttgaaagtatttcgatttcttggctttatatatcttgtggaaaggacgaaacaccgggcactcttccgtgatctggtggataaattcgcaagggtatcatggcggacgaccgggattcgaaccccggatccggccgtccgccgtgatccatgcggttaccgcccgcgtgtcgaacccaggtgtgcgacgtcagacaacgggggagcgctcctttttgggcccat

SEQ ID NO: 28 - DNA, 14058 bp, synthetic construct
  misc_feature 1..14058: note = source = /note="Description of Artificial Sequence: Synthetic polynucleotide"
  source 1..14058: mol_type = other DNA
tggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccactagtccatagagcccaccgcatccccagcatgcctgctattgtcttcccaatcctcccccttgctgtcctgccccaccccaccccctagaatagaatgacacctactcagacaatgcgatgcaatttcctcattttattaggaaaggacagtgggagtggcaccttccagggtcaaggaaggcacgggggaggggcaaacaacagatggctggcaactagaaggcacagctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcatgagacgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatgcactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgtcagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtctctgtctcaaaaggaggtaggcgatccctactgtacggagtgcgccgagacaaccgagatcgtgttggtcgtagtgtcatgccaaatggaacgccggacgtagtcatggttgtggccatattatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcttaaaaatcaaaggggttctgccgcgcatcactatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcgcttgtcgtcatcgtctttgtagtcgggaggcggcggcgacggcgacggggacgacacgtcctccatggttggtggacgtcgcgccgcaccgcgtccgcgctcgggggtggtttcgcgctgctcctcttcccgactggccatggtggccgaggataacttcgtatatggtttcttatacgaagttatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatctgctacgtatcaagctgtggcagggaaaccctctgcctcccccgtgatgtaatacttttgcaaggaatgcgatgaagtagagcccgcagtggccaagtggctttggtccgtctcctccacggatgcccctccacggctagtgggcgcatgtaggcggtgggcgtccgccgcctccagcagcaggtcatagaggggcaccacgttcttgcacttcatgctgtacagatgctccatgcctttgttactcatgtgtcggatgtgggagaggatgaggaggagctgggccagccgctggtgctgctgctgcagggtcaggcctgccttggccatcaggtggatcaaagtgtctgtgatcttgtccaggactcggtggatatggtccttctcttccagagacttcagggtgctggacagaaatgtgtacactccagaattaagcaaaataatagatttgaggcacacaaactcctctccctgcagattcatcatgcggaaccgagatgatgtagccagcagcatgtcgaagatctccaccatgccctctacacattttccctggttcctgtccaagagcaagttaggagcaaacagtagcttcactgggtgctccatggagcgccagacgagaccaatcatcaggatctctagccaggcacattctagaaggtggacctgatcatggagggtcaaatccacaaagcctggcaccctcttcgcccagttgatcatgtgaaccagctccctgtctgccaggttggtcagtaagcccatcatcgaagcttcactgaagggtctggtaggatcatactcggaatagagtatggggggctcagcatccaacaaggcactgaccatctggtcggccgtcagggacaaggccaggctgttcttcttagagcgtttgatcatgagcgggcttggccaaaggttggcagctctcatgtctccagcagatggctcgagatcgccatcttccagcaggcgcaccattgcccctgtttcactatccaggttacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatcctaaaatacacaaacaattagaatcagtagtttaacacattatacacttaaaaattttatatttaccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagtgactcccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacggacttggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcttgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacatggtggctacgtaataacttcgtatatggtttcttatacgaagttatgcggccgctttacgagggtaggaagtggtacggaaagttggtataagacaaaagtgttgtggaattgctccaggcgatctgacggttcactaaacgagctctgcttttataggcgcccaccgtacacgcctaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactcgcggccgcagagaaatgttctggcacctgcacttgcactggggacagcctattttgctagtttgttttgtttcgttttgttttgatggagagcgtatgttagtactatcgattcacacaaaaaaccaacacacagatgtaatgaaaataaagatattttattggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatgtaagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagcggatccgccgccaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctcttctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtgaaccggtcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttagagggcctatttcccatgattccttcatatttgcatatacgatacaaggctgttagagagataattagaattaatttgactgtaaacacaaagatattagtacaaaataataacttcgtataatgtatgctatacgaagttatcagacatgataagatacattgatgagtttggacaaaccacaactagaatgcagtgaaaaaaatgctttatttgtgaaatttgtgatgctattgctttatttgtaaccattataagctgcaataaacaaggtacctcaagcgccgggttttcgcgtcatgcaccacgtccgtggtcaaccctcccacacgtaaccagatgggagaagctctcgtattccaacagctgtaggttgaccgtctgaatccttcactatggcctttatacctggatgcagatccaacaggacttgtctacatctaccacaaggtgacaagatcccgcgattctcgttaccaatagcaacgatacaagtcagattccctgcggcggcggcagcggcggttcccagtactacgagttctgcgcaggggccacccgtaaaatgatacacatttaccccagtaaaaatccgcccatcggatgacaaggctgcactagccacggaatagtcgtcagatataggtatggaattgattgtggcggtagccctctctatcagcgtggactcctcctgagagaggggttttgccatggtggcggcttaagggttcgatcctctagagtccggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcattgggaaaagcgctcccctacccataacttcgtataatgtatgctatacgaagttattttgcagttttaaaattatgttttaaaatggactatcatatgcttaccgtaacttgaaagtatttcgatttcttggctttatatatcttgtggaaaggacgaaacaccgggcactcttccgtgatctggtggataaattcgcaagggtatcatggcggacgaccgggattcgaaccccggatccggccgtccgccgtgatccatgcggttaccgcccgcgtgtcgaacccaggtgtgcgacgtcagacaacgggggagcgctcctttttgggcccat

SEQ ID NO: 29 - DNA, 14067 bp, synthetic construct
  misc_feature 1..14067: note = source = /note="Description of Artificial Sequence: Synthetic polynucleotide"
  source 1..14067: mol_type = other DNA
tggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccactagtccatagagcccaccgcatccccagcatgcctgctattgtcttcccaatcctcccccttgctgtcctgccccaccccaccccctagaatagaatgacacctactcagacaatgcgatgcaatttcctcattttattaggaaaggacagtgggagtggcaccttccagggtcaaggaaggcacgggggaggggcaaacaacagatggctggcaactagaaggcacagctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcatgagacgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatgcactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgtcagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtctctgtctcaaaaggaggtaggcgatccctactgtacggagtgcgccgagacaaccgagatcgtgttggtcgtagtgtcatgccaaatggaacgccggacgtagtcatggttgtggccatattatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcttaaaaatcaaaggggttctgccgcgcatcactatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcgcttgtcgtcatcgtctttgtagtcgggaggcggcggcgacggcgacggggacgacacgtcctccatggttggtggacgtcgcgccgcaccgcgtccgcgctcgggggtggtttcgcgctgctcctcttcccgactggccatggtggccgaggataacttcgtatatggtttcttatacgaagttatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatctgctacgtatcaagctgtggcagggaaaccctctgcctcccccgtgatgtaatacttttgcaaggaatgcgatgaagtagagcccgcagtggccaagtggctttggtccgtctcctccacggatgcccctccacggctagtgggcgcatgtaggcggtgggcgtccgccgcctccagcagcaggtcatagaggggcaccacgttcttgcacttcatgctgtacagatgctccatgcctttgttactcatgtgtcggatgtgggagaggatgaggaggagctgggccagccgctggtgctgctgctgcagggtcaggcctgccttggccatcaggtggatcaaagtgtctgtgatcttgtccaggactcggtggatatggtccttctcttccagagacttcagggtgctggacagaaatgtgtacactccagaattaagcaaaataatagatttgaggcacacaaactcctctccctgcagattcatcatgcggaaccgagatgatgtagccagcagcatgtcgaagatctccaccatgccctctacacattttccctggttcctgtccaagagcaagttaggagcaaacagtagcttcactgggtgctccatggagcgccagacgagaccaatcatcaggatctctagccaggcacattctagaaggtggacctgatcatggagggtcaaatccacaaagcctggcaccctcttcgcccagttgatcatgtgaaccagctccctgtctgccaggttggtcagtaagcccatcatcgaagcttcactgaagggtctggtaggatcatactcggaatagagtatggggggctcagcatccaacaaggcactgaccatctggtcggccgtcagggacaaggccaggctgttcttcttagagcgtttgatcatgagcgggcttggccaaaggttggcagctctcatgtctccagcagatggctcgagatcgccatcttccagcaggcgcaccattgcccctgtttcactatccaggttacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatcctaaaatacacaaacaattagaatcagtagtttaacacattatacacttaaaaattttatatttaccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagtgactcccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacggacttggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcttgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacatggtggctacgtaataacttcgtatatggtttcttatacgaagttatgcggccgctttacgagggtaggaagtggtacggaaagttggtataagacaaaagtgttgtggaattgctccaggcgatctgacggttcactaaacgagctctgcttttataggcgcccaccgtacacgcctaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactcgcggccgcagagaaatgttctggcacctgcacttgcactggggacagcctattttgctagtttgttttgtttcgttttgttttgatggagagcgtatgttagtactatcgattcacacaaaaaaccaacacacagatgtaatgaaaataaagatattttattggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatgtaagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagcggatccgccgccaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctcttctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtgaaccggtcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttagagggcctatttcccatgattccttcatatttgcatatacgatacaaggctgttagagagataattagaattaatttgactgtaaacacaaagatattagtacaaaataataacttcgtataatgtatgctatacgaagttatcagacatgataagatacattgatgagtttggacaaaccacaactagaatgcagtgaaaaaaatgctttatttgtgaaatttgtgatgctattgctttatttgtaaccattataagctgcaataaacaaggtacctcaagcgccgggttttcgcgtcatgcaccacgtccgtggtctagaactagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaagagaattcgccgccaccatgaaaacatttaacatttctcaacaggatctagaattagtagaagtagcgacagagaagattacaatgctttatgaggataataaacatcatgtgggagcggcaattcgtacgaaaacaggagaaatcatttcggcagtacatattgaagcgtatataggacgagtaactgtttgtgcagaagccattgcgattggtagtgcagtttcgaatggacaaaaggattttgacacgattgtagctgttagacacccttattctgacgaagtagatagaagtattcgagtggtaagtccttgtggtatgtgtagggagttgatttcagactatgcaccagattgttttgtgttaatagaaatgaatggcaagttagtcaaaactacgattgaagaactcattccactcaaatatacccgaaattaaaccggtcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttagggcccattggtatggcttgggaaaagcgctcccctacccataacttcgtataatgtatgctatacgaagttattttgcagttttaaaattatgttttaaaatggactatcatatgcttaccgtaacttgaaagtatttcgatttcttggctttatatatcttgtggaaaggacgaaacaccgggcactcttccgtgatctggtggataaattcgcaagggtatcatggcggacgaccgggattcgaaccccggatccggccgtccgccgtgatccatgcggttaccgcccgcgtgtcgaacccaggtgtgcgacgtcagacaacgggggagcgctcctttttgggcccat

SEQ ID NO: 30 - DNA, 13923 bp, synthetic construct
  misc_feature 1..13923: note = source = /note="Description of Artificial Sequence: Synthetic polynucleotide"
  source 1..13923: mol_type = other DNA
tggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccactagtccatagagcccaccgcatccccagcatgcctgctattgtcttcccaatcctcccccttgctgtcctgccccaccccaccccctagaatagaatgacacctactcagacaatgcgatgcaatttcctcattttattaggaaaggacagtgggagtggcaccttccagggtcaaggaaggcacgggggaggggcaaacaacagatggctggcaactagaaggcacagctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcatgagacgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatgcactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgtcagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtctctgtctcaaaaggaggtaggcgatccctactgtacggagtgcgccgagacaaccgagatcgtgttggtcgtagtgtcatgccaaatggaacgccggacgtagtcatggttgtggccatattatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcttaaaaatcaaaggggttctgccgcgcatcactatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcgcttgtcgtcatcgtctttgtagtcgggaggcggcggcgacggcgacggggacgacacgtcctccatggttggtggacgtcgcgccgcaccgcgtccgcgctcgggggtggtttcgcgctgctcctcttcccgactggccatggtggccgaggataacttcgtatatggtttcttatacgaagttatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatctgctacgtatcaagctgtggcagggaaaccctctgcctcccccgtgatgtaatacttttgcaaggaatgcgatgaagtagagcccgcagtggccaagtggctttggtccgtctcctccacggatgcccctccacggctagtgggcgcatgtaggcggtgggcgtccgccgcctccagcagcaggtcatagaggggcaccacgttcttgcacttcatgctgtacagatgctccatgcctttgttactcatgtgtcggatgtgggagaggatgaggaggagctgggccagccgctggtgctgctgctgcagggtcaggcctgccttggccatcaggtggatcaaagtgtctgtgatcttgtccaggactcggtggatatggtccttctcttccagagacttcagggtgctggacagaaatgtgtacactccagaattaagcaaaataatagatttgaggcacacaaactcctctccctgcagattcatcatgcggaaccgagatgatgtagccagcagcatgtcgaagatctccaccatgccctctacacattttccctggttcctgtccaagagcaagttaggagcaaacagtagcttcactgggtgctccatggagcgccagacgagaccaatcatcaggatctctagccaggcacattctagaaggtggacctgatcatggagggtcaaatccacaaagcctggcaccctcttcgcccagttgatcatgtgaaccagctccctgtctgccaggttggtcagtaagcccatcatcgaagcttcactgaagggtctggtaggatcatactcggaatagagtatggggggctcagcatccaacaaggcactgaccatctggtcggccgtcagggacaaggccaggctgttcttcttagagcgtttgatcatgagcgggcttggccaaaggttggcagctctcatgtctccagcagatggctcgagatcgccatcttccagcaggcgcaccattgcccctgtttcactatccaggttacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatcctaaaatacacaaacaattagaatcagtagtttaacacattatacacttaaaaattttatatttaccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagtgactcccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacggacttggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcttgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacatggtggctacgtaataacttcgtatatggtttcttatacgaagttatgcggccgctttacgagggtaggaagtggtacggaaagttggtataagacaaaagtgttgtggaattgctccaggcgatctgacggttcactaaacgagctctgcttttataggcgcccaccgtacacgcctaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactcgcggccgcagagaaatgttctggcacctgcacttgcactggggacagcctattttgctagtttgttttgtttcgttttgttttgatggagagcgtatgttagtactatcgattcacacaaaaaaccaacacacagatgtaatgaaaataaagatattttattggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatgtaagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagcggatccgccgccaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctcttctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtgaaccggtcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttagagggcctatttcccatgattccttcatatttgcatatacgatacaaggctgttagagagataattagaattaatttgactgtaaacacaaagatattagtacaaaataataacttcgtataatgtatgctatacgaagttatcagacatgataagatacattgatgagtttggacaaaccacaactagaatgcagtgaaaaaaatgctttatttgtgaaatttgtgatgctattgctttatttgtaaccattataagctgcaataaacaaggtacctcaagcgccgggttttcgcgtcatgcaccacgtccgtggttcaagcgccgggttttcgcgtcatgcaccacgtccgtgggccctcgggtacttcaacgtcagcagtaactgtaaatccgagccgttcatagaagggcaaattccttggcgctgacgtttcaagaaaggctggcactccggctcgttctgcggcttctactccgggcaataccaccgcggaaccaaggccctttccctgatgatcggggctaacgcccacagtagcgaggaaccaagctggttctttagggcggtgaggggcgaggagtccttccatttgttgctgagccgcgagacgagagccactaagctcagccattcggggaccaatttctgcaaatacagccccggcctcaacgctctccggagtcgtccacactgccactgcagccccgtcgtcggcgacccaaactttaccgatgtccaatcctaccctggtcaaaaaaagttcttgcaattctgtaacccgttcaatatgtctatcaggatcaactgtgtggcgtgtagcgggataatccgcgaaagcggcagccaatgttctcacggccctagggacgtcgtctcgagttgccagtctgacagtaggtttatattctgtcatggtggcggcgaattctcttctatggaggtcaaaacagcgtggatggcgtctccaggcgatctgacggttcactaaacgagctctgcttatataaacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatactagttcttgggaaaagcgctcccctacccataacttcgtataatgtatgctatacgaagttattttgcagttttaaaattatgttttaaaatggactatcatatgcttaccgtaacttgaaagtatttcgatttcttggctttatatatcttgtggaaaggacgaaacaccgggcactcttccgtgatctggtggataaattcgcaagggtatcatggcggacgaccgggattcgaaccccggatccggccgtccgccgtgatccatgcggttaccgcccgcgtgtcgaacccaggtgtgcgacgtcagacaacgggggagcgctcctttttgggcccat

SEQ ID NO: 31 - DNA, 14372 bp, synthetic construct
  misc_feature 1..14372: note = source = /note="Description of Artificial Sequence: Synthetic polynucleotide"
  source 1..14372: mol_type = other DNA
actcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccactagtccatagagcccaccgcatccccagcatgcctgctattgtcttcccaatcctcccccttgctgtcctgccccaccccaccccctagaatagaatgacacctactcagacaatgcgatgcaatttcctcattttattaggaaaggacagtgggagtggcaccttccagggtcaaggaaggcacgggggaggggcaaacaacagatggctggcaactagaaggcacagctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcatgagacgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatgcactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgtcagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtctctgtctcaaaaggaggtaggcgatccctactgtacggagtgcgccgagacaaccgagatcgtgttggtcgtagtgtcatgccaaatggaacgccggacgtagtcatggttgtggccatattatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcttaaaaatcaaaggggttctgccgcgcatcactatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcgcttgtcgtcatcgtctttgtagtcgggaggcggcggcgacggcgacggggacgacacgtcctccatggttggtggacgtcgcgccgcaccgcgtccgcgctcgggggtggtttcgcgctgctcctcttcccgactggccatggtggccgaggataacttcgtatatggtttcttatacgaagttatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatctgctacgtatcaagctgtggcagggaaaccctctgcctcccccgtgatgtaatacttttgcaaggaatgcgatgaagtagagcccgcagtggccaagtggctttggtccgtctcctccacggatgcccctccacggctagtgggcgcatgtaggcggtgggcgtccgccgcctccagcagcaggtcatagaggggcaccacgttcttgcacttcatgctgtacagatgctccatgcctttgttactcatgtgtcggatgtgggagaggatgaggaggagctgggccagccgctggtgctgctgctgcagggtcaggcctgccttggccatcaggtggatcaaagtgtctgtgatcttgtccaggactcggtggatatggtccttctcttccagagacttcagggtgctggacagaaatgtgtacactccagaattaagcaaaataatagatttgaggcacacaaactcctctccctgcagattcatcatgcggaaccgagatgatgtagccagcagcatgtcgaagatctccaccatgccctctacacattttccctggttcctgtccaagagcaagttaggagcaaacagtagcttcactgggtgctccatggagcgccagacgagaccaatcatcaggatctctagccaggcacattctagaaggtggacctgatcatggagggtcaaatccacaaagcctggcaccctcttcgcccagttgatcatgtgaaccagctccctgtctgccaggttggtcagtaagcccatcatcgaagcttcactgaagggtctggtaggatcatactcggaatagagtatggggggctcagcatccaacaaggcactgaccatctggtcggccgtcagggacaaggccaggctgttcttcttagagcgtttgatcatgagcgggcttggccaaaggttggcagctctcatgtctccagcagatggctcgagatcgccatcttccagcaggcgcaccattgcccctgtttcactatccaggttacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatcctaaaatacacaaacaattagaatcagtagtttaacacattatacacttaaaaattttatatttaccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagtgactcccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacggacttggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcttgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacatggtggctacgtaataacttcgtatatggtttcttatacgaagttatgcggccgctttacgagggtaggaagtggtacggaaagttggtataagacaaaagtgttgtggaattgctccaggcgatctgacggttcactaaacgagctctgcttttataggcgcccaccgtacacgcctaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactcgcggccgcagagaaatgttctggcacctgcacttgcactggggacagcctattttgctagtttgttttgtttcgttttgttttgatggagagcgtatgttagtactatcgattcacacaaaaaaccaacacacagatgtaatgaaaataaagatattttattggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatgtaagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagcggatccgccgccaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctcttctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtgaaccggtcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttagagggcctatttcccatggtggtagaactagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaagagaattcgccgccaccatgacagaatataaacctactgtcagactggcaactcgagacgacgtccctagggccgtgagaacattggctgccgctttcgcggattatcccgctacacgccacacagttgatcctgatagacatattgaacgggttacagaattgcaagaactttttttgaccagggtaggattggacatcggtaaagtttgggtcgccgacgacggggctgcagtggcagtgtggacgactccggagagcgttgaggccggggctgtatttgcagaaattggtccccgaatggctgagcttagtggctctcgtctcgcggctcagcaacaaatggaaggactcctcgcccctcaccgccctaaagaaccagcttggttcctcgctactgtgggcgttagccccgatcatcagggaaagggccttggttccgcggtggtattgcccggagtagaagccgcagaacgagccggagtgccagcctttcttgaaacgtcagcgccaaggaatttgcccttctatgaacggctcggatttacagttactgctgacgttgaagtacccgagggcccacggacgtggtgcatgacgcgaaaacccggcgcttgagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttagtttaaacgggcccttaattaatcgatgtaggatgttgcccctcctgacgcggtaggagaaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggcccgagactcgaaccgggggtcctgcgactcaacccttggaaaataaccctccggctacagggagcgagccacttaatgctttcgctttccagcctaaccgcttacgccgcgcgcggccagtggccaaaaaagctagcgcagcagccgccgcgcctggaaggaagccaaaaggagcgctcccccgttgtctgacgtcgcacacctgggttcgacacgcgggcggtaaccgcatggatcacggcggacggccggatccggggttcgaaccccggtcgtccgccatgatacccttgcgaatttatccaccagaccacggaagagtgcccgcttacaggctctccttttgcacggtctagagcgtcaacgactgcgcacgcctcaccggccagagcgtcccgaccatggagcactttttgccgctgcgcaacatctggaaccgcgtccgcgactttccgcgcgcctccaccaccgccgccggcatcacctggatgtccaggtacatctacggattacggggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 32 - DNA, 11646 bp, synthetic construct
  misc_feature 1..11646: note = source = /note="Description of Artificial Sequence: Synthetic polynucleotide"
  source 1..11646: mol_type = other DNA
actcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatggaggggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccgttgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacataacttcgtataaagtatactatacgaagttatcgggcccctctgctaaccatgttcatgccttcttctttttcctacagatgtcagaactcattaaagagaatatgcacatgaagctgtatatggaaggtactgtagacaaccaccatttcaaatgcacgtccgaaggtgaggggaagccatacgagggtacccaaactatgcgcatcaaagtggttgagggtggccccctgccattcgcattcgacatcctggcaactagctttctttacggttccaagacattcataaatcatacccagggtattcccgatttcttcaaacaatccttcccggaagggtttacttgggagcgggtcacgacatatgaagacgggggtgttcttacagccacacaggatacgagtttgcaagacggttgtcttatctataacgtgaagattcggggtgtgaatttcacatccaatggcccggtgatgcagaaaaaaacactgggctgggaagcatttacggagacgttgtatcccgccgatggaggtctcgagggccgaaacgatatggccctcaagttggtaggtggttctcaccttatagcaaacattaagaccacgtatcgatcaaaaaaacccgctaagaatctgaaaatgccaggcgtgtattatgttgattacagactggagcgaataaaagaggctaacaatgagacctacgtcgaacagcatgaagtcgctgtagctagatattgcgacctcccgtcaaagttgggccataaattgaattaacctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcataacttcgtataaagtatactatacgaagttataattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctttttcctacaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttgtaaataaatttagtagtcatgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaattgcttgttaatcaataaaccgtttaattcgtttcagttgaactttggtctctgcgtatttctttcttatctagtttccatggctacgtagataagtagcatggcgggttaatcattaactacagcccgggcgtttaaacagcgggcggaggggtggagtcgtgacgtgaattacgtcatagggttagggaggtcctgtattagaggtcacgtgagtgttttgcgacattttgcgacaccatgtggtccgcggccgcaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgatatcgccaccatgattaagatcgctacgcggaagtacctggggaaacagaacgtctacgacataggtgtggagcgcgatcacaactttgctctgaaaaatggatttatcgccagcaactgtagggagttgatttcagactatgcaccagattgttttgtgttaatagaaatgaatggcaagttagtcaaaactacgattgaagaactcattccactcaaatatacccgaaattaagtgcatgacccgcaagcccggtgcctgaaatcaacctctggattacaaaatttgtgaaagattgactggtattcttaactatgttgctccttttacgctatgtggatacgctgctttaatgcctttgtatcatgcgttaactaaacttgtttattgcagcttataatggttacaaataaagcaatagcatcacaaatttcacaaataaagcatttttttcactgcattctagttgtggtttgtccaaactcatcaatgtatcttatcatgtctggaattgactcaaatgatgtcaattagtctatcagaagctcatctggtctcccttccgggggacaagacatccctgtttaatatttaaacagcagtgttcccaaactgggttcttatatcccttgctctggtcaaccaggttgcagggtttcctgtcctcacaggaacgaagtccctaaagaaacagtggcagccaggtttagccccggaattgactggattccttttttagggcccattggtatggcgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 33 - DNA, 8190 bp, synthetic construct
  misc_feature 1..8190: note = source = /note="Description of Artificial Sequence: Synthetic polynucleotide"
  source 1..8190: mol_type = other DNA
actcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctctgcagccgcgaccggccaaggtttaatgataggctgcaacgggatgttgggaatatgttgcactggtccgtgagggtaccaacttgtttattgcagcttataatggttacaaataaagcaatagcatcacaaatttcacaaataaagcatttttttcactgcattctagttgtggtttgtccaaactcatcaatgtatcttatcatgtctgaccggttcacttgagctcgagatctgagtacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcggcttaagggttcgatcctctagagtccggaggctggatcggtcccggtgtctactatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgcgtatggtacctgcggaggatgccgaggataaccttgttactagcctccgcctggccgttggactgtggataatatggcgtagaggatcctctgcgcgctcgctcgctcactgaggccgcccgggcaaagcccgggcgtcgggcgacctttggtcgcccggcctcagtgagcgagcgagcgcgcagagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggtctagagctagcgaattcgaatttaaatcggatccgcggccgcaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgatatcgccaccatgaaaacatttaacatttctcaacaggatctagaattagtagaagtagcgacagagaagattacaatgctttatgaggataataaacatcatgtgggagcggcaattcgtacgaaaacaggagaaatcatttcggcagtacatattgaagcgtatataggacgagtaactgtttgtgcagaagccattgcgattggtagtgcagtttcgaatggacaaaaggattttgacacgattgtagctgttagacacccttattctgacgaagtagatagaagtattcgagtggtaagtccttgtggtatgtgcctttcatacgagaccgagatcctgactgtcgagtacggattgcttcctatcggcaaaatcgtggagaagaggattgaatgtaccgtctattcagtcgataataatgggaacatctacacacagcccgtggctcaatggcacgacagaggagagcaggaagtttttgaatactgtctcgaggacggatccctcatccgcgctactaaagatcataagtttatgaccgtggacggccagatgctgccaattgacgaaatttttgaacgagagctggatctgatgagagtcgacaaccttccaaactgagtgcatgacccgcaagcccggtgcctgaaatcaacctctggattacaaaatttgtgaaagattgactggtattcttaactatgttgctccttttacgctatgtggatacgctgctttaatgcctttgtatcatgcgttaactaaacttgtttattgcagcttataatggttacaaataaagcaatagcatcacaaatttcacaaataaagcatttttttcactgcattctagttgtggtttgtccaaactcatcaatgtatcttatcatgtctggaattgactcaaatgatgtcaattagtctatcagaagctatctggtctcccttccgggggacaagacatccctgtttaatatttaaacagcagtgttcccaaactgggttcttatatcccttgctctggtcaaccaggttgcagggtttcctgtcctcacaggaacgaagtccctaaagaaacagtggcagccaggtttagccccggaattgactggattccttttttagggcccattggtatggctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 34 - DNA, 4639 bp, synthetic construct
  misc_feature 1..4639: note = source = /note="Description of Artificial Sequence: Synthetic polynucleotide"
  source 1..4639: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattctctgcgcgctcgctcgctcactgaggccgggcgaccaaaggtcgcccgacgcccgggctttgcccgggcggcctcagtgagcgagcgagcgcgcagaggatcctctacgccatattatccacagtccaacggccaggcggaggctagtaacaaggttatcctcggcatcctccgcaggtaccatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagtagacaccgggaccgatccagcctccggactctagaggatcgaacccttaagccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtactcagatctcgagctcaagtgaaccggtcagacatgataagatacattgatgagtttggacaaaccacaactagaatgcagtgaaaaaaatgctttatttgtgaaatttgtgatgctattgctttatttgtaaccattataagctgcaataaacaagttggtaccctcacggaccagtgcaacatattcccaacatcccgttgcagcctatcattaaaccttggccggtcgcggctgcagaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaaaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 35 - DNA, 10898 bp, synthetic construct
  misc_feature 1..10898: note = source = /note="Description of Artificial Sequence: Synthetic polynucleotide"
  source 1..10898: mol_type = other DNA
ggtacccaactccatgcttaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactaggagacactttcaataaaggcaaatgtttttatttgtacactctcgggtgattatttaccccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcggggaggcggcggcgacggcgacggggacgacacgtcctccatggttggtggacgtcgcgccgcaccgcgtccgcgctcgggggtggtttcgcgctgctcctcttcccgactggccatttccttctcctataggcagaaaaagatcatggagtcagtcgagaaggaggacagcctaaccgccccctttgagttcgccaccaccgcctccaccgatgccgccaacgcgcctaccaccttccccgtcgaggcacccccgcttgaggaggaggaagtgattatcgagcaggacccaggttttgtaagcgaagacgacgaggatcgctcagtaccaacagaggataaaaagcaagaccaggacgacgcagaggcaaacgaggaacaagtcgggcggggggaccaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctgttctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcgacgaagtgccaaaaatctttgagggtcttggacgcgacgagaaacgcgcggcaaacgctctgcaacaagaaaacagcgaaaatgaaagtcactgtggagtgctggtggaacttgagggtgacaacgcgcgcctagccgtgctgaaacgcagcatcgaggtcacccactttgcctacccggcacttaacctaccccccaaggttatgagcacagtcatgagcgagctgatcgtgcgccgtgcacgacccctggagagggatgcaaacttgcaagaacaaaccgaggagggcctacccgcagttggcgatgagcagctggcgcgctggcttgagacgcgcgagcctgccgacttggaggagcgacgcaagctaatgatggccgcagtgcttgttaccgtggagcttgagtgcatgcagcggttctttgctgacccggagatgcagcgcaagctagaggaaacgttgcactacacctttcgccagggctacgtgcgccaggcctgcaaaatttccaacgtggagctctgcaacctggtctcctaccttggaattttgcacgaaaaccgcctcgggcaaaacgtgcttcattccacgctcaagggcgaggcgcgccgcgactacgtccgcgactgcgtttacttatttctgtgctacacctggcaaacggccatgggcgtgtggcagcaatgcctggaggagcgcaacctaaaggagctgcagaagctgctaaagcaaaacttgaaggacctatggacggccttcaacgagcgctccgtggccgcgcacctggcggacattatcttccccgaacgcctgcttaaaaccctgcaacagggtctgccagacttcaccagtcaaagcatgttgcaaaactttaggaactttatcctagagcgttcaggaattctgcccgccacctgctgtgcgcttcctagcgactttgtgcccattaagtaccgtgaatgccctccgccgctttggggtcactgctaccttctgcagctagccaactaccttgcctaccactccgacatcatggaagacgtgagcggtgacggcctactggagtgtcactgtcgctgcaacctatgcaccccgcaccgctccctggtctgcaattcgcaactgcttagcgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacatccttggccaattgcaagccatcaacaaagcccgccaagagtttctgctacgaaagggacggggggtttacctggacccccagtccggcgaggagctcaacccaatccccccgccgccgcagccctatcagcagccgcgggcccttgcttcccaggatggcacccaaaaagaagctgcagctgccgccgccgccacccacggacgaggaggaatactgggacagtcaggcagaggaggttttggacgaggaggaggagatgatggaagactgggacagcctagacgaagcttccgaggccgaagaggtgtcagacgaaacaccgtcaccctcggtcgcattcccctcgccggcgccccagaaattggcaaccgttcccagcatcgctacaacctccgctcctcaggcgccgccggcactgcctgttcgccgacccaaccgtagatgggacaccactggaaccagggccggtaagtctaagcagccgccgccgttagcccaagagcaacaacagcgccaaggctaccgctcgtggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccctactgcaccggcggcagcggcagcggcagcaacagcagcggtcacacagaagcaaaggcgaccggatagcaagactctgacaaagcccaagaaatccacagcggcggcagcagcaggaggaggagcgctgcgtctggcgcccaacgaacccgtatcgacccgcgagcttagaaataggatttttcccactctgtatgctatatttcaacaaagcaggggccaagaacaagagctgaaaataaaaaacaggtctctgcgctccctcacccgcagctgcctgtatcacaaaagcgaagatcagcttcggcgcacgctggaagacgcggaggctctcttcagcaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcctcgaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcgttttagggcggagtaacttgcatgtattgggaattgtagtttttttaaaatgggaagtgacgtatcgtgggaaaacggaagtgaagatttgaggaagttgtgggttttttggctttcgtttctgggcgtaggttcgcgtgcggttttctgggtgttttttgtggactttaaccgttacgtcattttttagtcctatatatactcgctctgtacttggccctttttacactgtgactgattgagctggtgccgtgtcgagtggtgttttttaataggtttttttactggtaaggctgactgttatggctgccgctgtggaagcgctgtatgttgttctggagcgggagggtgctattttgcctaggcaggagggtttttcaggtgtttatgtgtttttctctcctattaattttgttatacctcctatgggggctgtaatgttgtctctacgcctgcgggtatgtattcccccgggctatttcggtcgctttttagcactgaccgatgttaaccaacctgatgtgtttaccgagtcttacattatgactccggacatgaccgaggaactgtcggtggtgctttttaatcacggtgaccagtttttttacggtcacgccggcatggccgtagtccgtcttatgcttataagggttgtttttcctgttgtaagacaggcttctaatgtttaaatgtttttttttttgttattttattttgtgtttaatgcaggaacccgcagacatgtttgagagaaaaatggtgtctttttctgtggtggttccggaacttacctgcctttatctgcatgagcatgactacgatgtgcttgcttttttgcgcgaggctttgcctgattttttgagcagcaccttgcattttatatcgccgcccatgcaacaagcttacataggggctacgctggttagcatagctccgagtatgcgtgtcataatcagtgtgggttcttttgtcatggttcctggcggggaagtggccgcgctggtccgtgcagacctgcacgattatgttcagctggccctgcgaagggacctacgggatcgcggtatttttgttaatgttccgcttttgaatcttatacaggtctgtgaggaacctgaatttttgcaatcatgattcgctgcttgaggctgaaggtggagggcgctctggagcagatttttacaatggccggacttaatattcgggatttgcttagagacatattgataaggtggcgagatgaaaattatttgggcatggttgaaggtgctggaatgtttatagaggagattcaccctgaagggtttagcctttacgtccacttggacgtgagggcagtttgccttttggaagccattgtgcaacatcttacaaatgccattatctgttctttggctgtagagtttgaccacgccaccggaggggagcgcgttcacttaatagatcttcattttgaggttttggataatcttttggaataaaaaaaaaaaaacatggttcttccagctcttcccgctcctcccgtgtgtgactcgcagaacgaatgtgtaggttggctgggtgtggcttattctgcggtggtggatgttatcagggcagcggcgcatgaaggagtttacatagaacccgaagccagggggcgcctggatgctttgagagagtggatatactacaactactacacagagcgagctaagcgacgagaccggagacgcagatctgtttgtcacgcccgcacctggttttgcttcaggaaatatgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttccccttcgccgcccgttgagcaaccgcaagttggacagcagcctgtggctcagcagctggacagcgacatgaacttaagcgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatcaatataagctatgtggtggtggggctatactactgaatgaaaaatgacttgaaattttctgcaattgaaaaataaacacgttgaaacataacatgcaacaggttcacgattctttattcctgggcaatgtaggagaaggtgtaagagttggtagcaaaagtttcagtggtgtattttccactttcccaggaccatgtaaaagacatagagtaagtgcttacctcgctagtttctgtggattcactagaatcgatgtcgacgtttaaaccatatgatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgatggatcc

SEQ ID NO: 36 - RNA, 36 nt, synthetic construct
  misc_feature 1..36: note = source = /note="Description of Artificial Sequence: Synthetic oligonucleotide"
  source 1..36: mol_type = other RNA
ggacgaccggggttcgaaccccggatccggccgtcc

SEQ ID NO: 37 - RNA, 68 nt, synthetic construct
  misc_feature 1..3: note = /note="This region may or may not be present"
  misc_feature 1..68: note = source = /note="Description of Artificial Sequence: Synthetic oligonucleotide"
  misc_feature 1..68: note = source = /note="See specification as filed for detailed description of substitutions and preferred embodiments"
  source 1..68: mol_type = other RNA
agcgggcactcttccgtggtctggtggataaattcgcaagggtatcatggcggacgaccggggttcga

SEQ ID NO: 38 - RNA, 95 nt, synthetic construct
  misc_feature 91..95: note = /note="This region may encompass 0-5 nucleotides"
  misc_feature 1..95: note = source = /note="Description of Artificial Sequence: Synthetic oligonucleotide"
  source 1..95: mol_type = other RNA
cccggatccggccgtccgccgtgatccatgcggttaccgcccgcgtgtcgaacccaggtgtgcgacgtcagacaacgggggagcgctcctttttt

SEQ ID NO: 39 - skipped

SEQ ID NO: 40 - RNA, 160 nt, synthetic construct
  source 1..160: mol_type = other RNA
aggggcactcttccgtggtctgaggtggataaattcgcaagggtatcatggcggacgaccggggttcgacccggatccggccgtccgccgtgatccatgcggttaccgcccgcgtgtcgaacccaggtgtgcgacgtcagacaacgggggagcgctcctt

SEQ ID NO: 41 - RNA, 36 nt, synthetic construct; identical to SEQ ID NO: 36
  source 1..36: mol_type = other RNA